(19) United States
(12) Patent Application Publication
(10) Pub. No.: US 2026/0232783 A1
(43) Pub. Date: Aug. 13, 2026

(54) COMPOSITIONS FOR USE IN TREATMENT OF ACNE

(71) Applicant: SANOFI PASTEUR INC., Swiftwater, PA (US)

(72) Inventors: Nadège ARNAUD BARBE, Paris (FR); Danilo CASIMIRO, Cambridge, MA (US); Andreas KARLSSON, Paris (FR); Isabelle LEGASTELOIS, Paris (FR); Noëlle MISTRETTA, Paris (FR); Geneviève RENAULD, Paris (FR); Bachra MOHAMED ROKBI, Paris (FR); Khang TRAN, Cambridge, MA (US); Monica WU, Cambridge, MA (US)

(21) Appl. No.: 19/379,080

(22) Filed: Nov. 4, 2025

Related U.S. Application Data
(63) Continuation of application No. 18/654,557, filed on May 3, 2024, now Pat. No. 12,502,424.
(60) Provisional application No. 63/464,523, filed on May 5, 2023.

(30) Foreign Application Priority Data
Jun. 29, 2023 (EP) 23306076.3
Nov. 8, 2023 (EP) 23306927.7

(51) Int. Cl. A61K 39/05; A61K 39/00; A61P 37/04; C07K 14/005; C07K 14/195

(52) U.S. Cl. CPC A61K 39/05; A61P 37/04; C07K 14/005; C07K 14/195; A61K 2039/53; A61K 2039/6018; A61K 2039/70; C07K 2319/03

(57) ABSTRACT

This invention relates to compositions (e.g. immunogenic compositions) which can be used to immunise against C. acnes. The compositions comprise C. acnes antigens and antigen combinations, used in the form of nucleic acids (e.g. mRNAs) encoding antigenic proteins or in the form of recombinant protein antigens.

### RELATED APPLICATIONS

This application is a divisional of U.S. patent application Ser. No. 18/654,557, filed May 3, 2024, which claims priority to U.S. Provisional Patent Application Ser. No. 63/464,523, filed May 5, 2023, European Patent Application Nos. 23306927.7, filed Nov. 8, 2023, and 23306076.3, filed Jun. 29, 2023, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Oct. 13, 2025, is named 770791_SA9-378 DIV_ST26.xml and is 533,110 bytes in size.

### FIELD OF THE INVENTION

The invention is in the field of treating and preventing Cutibacterium acnes (C. acnes) infections, such as acne vulgaris. In particular, the invention relates to antigens and antigen combinations which can be used to immunise against C. acnes, used in the form of nucleic acids (e.g. mRNAs) encoding antigenic proteins or in the form of recombinant protein antigens.

### BACKGROUND

Cutibacterium acnes (C. acnes, previously named Propionibacterium acnes) is a gram-positive bacterium known for its role in skin disorders such as acne vulgaris. C. acnes is a skin commensal which predominantly resides within sebaceous follicles which provide a unique lipid-rich environment due to secretion of sebum. Apart from skin, C. acnes has also been found in the conjunctiva, respiratory tract, genitourinary tract and gastrointestinal tract of humans and other animals.

Acne is a disease of pilosebaceous units in the skin, affecting more than 85% of adolescents and more than 20% of population continues to experience symptoms well beyond the teenage period. Acne vulgaris manifests in different severity grades: mild, moderate and severe. Moderate and severe acne account for more than one third of all cases and require medical treatment. Acne can appear also after the puberty, as an adult-onset condition, often associated with hormonal fluctuations which are more prevalent in women. C. acnes is also thought to play a role in other severe types of acne, such as acne conglobata, acne fulminans and cystic acne. In addition to dermatological pathology, C. acnes has also been found in corneal ulcers, and is a common cause of chronic endophthalmitis following cataract surgery. Various other inflammatory diseases have been associated with C. acnes, including postoperative prosthetic implant- and device-related infections (implant-associated infections), endocarditis, sarcoidosis, osteomyelitis, allergic alveolitis, pulmonary angitis, the SAPHO syndrome (synovitis, acne, pustulosis, hyperostosis, osteitis) and inflammation of lumbar nerve roots leading to sciatica. More recent studies suggest potential pathogenetic role of C. acnes also in non-infectious diseases such as prostatic cancer, due to its ability to persist intracellularly which may lead to altered gene expression.

Phylogenetic studies based on multilocus gene sequencing, as well as whole-genome analyses of isolates from the Human Microbiome Project (HMP) have provided valuable insights into the genetic population structure of C. acnes. The Multi Locus Sequence Typing (MLST) generates different sequences defined as distinct alleles which are then used to generate sequence types (STs) for every C. acnes isolate. Two different MLST typing schemes have been developed which divide C. acnes into closely related clusters IA1, IA2, IB, IC, II, and III (McDowell et al. 2012) or I-1a, I-1b, I-2, II, and III (Lomholt and Kilian 2010), according to MLST8 or MLST9 typing schemes, respectively. There has been some suggestion that only the strains of type IA I should be targeted in the context of a therapeutic (McLaughlin et al. 2019-page 23; O'Neill and Gallo 2018-page 4, second column, end of the first long paragraph), as these are most commonly isolated from the skin of acne patients, whereas others can be found on both acne-prone and healthy skin. However, other phylotypes have also been shown to be associated with acne (WO2021/165543). For example, C. acnes strains from phylotypes IA2, IC or II have been isolated from inflamed lesions of human subjects (WO2021/165543).

C. acnes strains can be divided into groups using genomic sequencing of 16S rDNA sequence called a ribotype (RT). This system allowed comparison of the C. acnes strain populations in individuals based on the 16S rDNA sequences. The top 10 major ribotypes were highly abundant while also a significant number of rare ribotypes were identified. According to the analysis of the top 10 ribotypes, both disease-specific and health-specific associations could be identified (Fitz-Gibbon et al. 2013; Tomida et al. 2013; Mclaughlin et al. 2019). The three most abundant ribotypes (RT1, RT2, and RT3) were fairly evenly distributed among acne and normal individuals. However, ribotypes RT4, RT5, RT7, RT8, RT9, and RT10 were found predominantly in acne patients, while RT6 was strongly associated with normal skin. A phylogenetic tree based on unique single-nucleotide polymorphism positions in the core genome obtained from these 71 C. acnes genomes suggested that the 16S rDNA ribotypes to a large extent represent the relationship of the lineages, and that the 16S rDNA sequence is a useful molecular marker to distinguish major C. acnes lineages (Fitz-Gibbon et al. 2013; Tomida et al. 2013).

Current treatments target one or two steps in acne pathogenesis, including benzoyl peroxide, topical retinoids, and topical or oral antibiotics. Nevertheless, antibiotic resistance is a concern in acne. In addition, oral isotretinoin, which is recommended in more severe cases, is associated with adverse effects, the most important one being teratogenicity. There is a need for an effective C. acnes vaccine.

### DISCLOSURE OF THE INVENTION

The present inventors have provided C. acnes antigens and antigen combinations which can be used to immunise against C. acnes.

In particular, the inventors showed that antigens derived from C. acnes CAMP2, DsA1, DsA2 and/or PITP polypeptides, alone or in combination, elicited an antibody response when delivered by mRNAs encoding the relevant antigens or in the form of recombinant polypeptides.

Accordingly, the invention provides C. acnes CAMP2 polypeptides, modified C. acnes CAMP2 polypeptides, C. acnes DsA1 polypeptides, C. acnes DsA2 polypeptides, C. acnes PITP polypeptides, chimeric C. acnes DsA1/DsA2 polypeptides, chimeric C. acnes DsA1/DsA2/PITP polypeptides and chimeric C. acnes DsA1/DsA2/PITP/CAMP2 polypeptides and nucleic acids comprising a nucleotide sequence encoding such polypeptides.

Polypeptide antigens described herein may be delivered by, i.e. in the form of, a nucleic acid (e.g. mRNA) comprising a nucleotide sequence encoding said polypeptide or as, i.e. in the form of, a recombinant protein.

### CAMP2

Christie-Atkins-Munch-Petersen (CAMP) factor 2, or CAMP2, is a polypeptide belonging to the CAMP factor superfamily, whose members are known for their co-hemolytic and cytotoxic properties. C. acnes CAMP factors 1-5 (or CAMP1-5) are thought to be the causative agents of a co-hemolytic reaction of C. acnes with both sheep and human erythrocytes (Choudhury, 1978, Sörensen et al, Journal of Microbiological Methods (2010) 83 (2): 211-216). The amino acid sequences of C. acnes CAMP factors 1-5 have a low degree of sequence identity between them (e.g. around 50% for CAMP2 and CAMP4 amino acid sequences and even less for others). Native C. acnes CAMP2 polypeptide is a secreted protein.

CAMP2 has been identified as a putative acne-associated virulence factor (Holland et al (2010)). The expression and secretion of CAMP proteins by different C. acnes strains has been suggested as one of the factors contributing to C. acnes resistance to opsonophagocytic killing. It has been reported that CAMP2 is able to induce killing of phagocytic cells in an in vitro assay involving co-cultivation with C. acnes (Wang et al. 2018). Furthermore, anti-CAMP2 neutralizing antibodies were shown to significantly decrease inflammation induced by C. acnes in a mouse ear model (Liu et al. 2011, Vaccine, 29:3230-3238).

The inventors have recognised that C. acnes CAMP2 polypeptides may be used to elicit an immune response against a C. acnes infection. In particular, the inventors have demonstrated that C. acnes CAMP2 polypeptides elicit an antibody (e.g. IgG) response. Notably, C. acnes CAMP2 polypeptides elicited an antibody (e.g. IgG) response, when delivered as a mRNA encoding the antigen, as well as in the form of recombinant protein. Furthermore, the inventors demonstrated that an antibody (e.g. IgG) response was elicited when a mRNA encoding a C. acnes CAMP2 polypeptide was delivered in lipid nanoparticle (LNP). Antibodies induced by C. acnes CAMP2 polypeptides reduced the co-hemolytic activity of CAMP2. C. acnes CAMP2 polypeptides may therefore elicit antibodies in a subject, e.g. antibodies that can neutralise biological activity of CAMP2 polypeptide, such as CAMP2 inflammatory activity.

The inventors have therefore demonstrated that C. acnes CAMP2 polypeptides (e.g. delivered in the form of a mRNA encoding the antigen or as a recombinant protein) are suitable vaccine antigens that may be used alone, in combination with other C. acnes antigens described herein, such as one or more of C. acnes DsA1 polypeptides, C. acnes DsA2 polypeptides, C. acnes PITP polypeptides, chimeric C. acnes DsA1/DsA2 polypeptides and chimeric C. acnes DsA1/DsA2/PITP polypeptides (e.g. delivered in the form of a mRNA encoding the antigen or as a recombinant protein) described herein, or as chimeric C. acnes DsA1/DsA2/PITP/CAMP2 polypeptides. CAMP2 polypeptides may elicit antibodies that neutralise CAMP2 inflammatory activity whilst immunisation with one or more of these other C. acnes antigens may elicit antibodies that bind to the surface of C. acnes bacteria and recruit effector cells (e.g. effector cells of the immune system (e.g. innate immune system) such as phagocytes).

Accordingly, in one aspect, the invention provides a nucleic acid that comprises a nucleotide sequence encoding a C. acnes CAMP2 polypeptide. Typically, the nucleic acid is a messenger RNA (mRNA). In a preferred embodiment, the nucleic acid is a non-naturally occurring nucleic acid, e.g. a non-naturally occurring mRNA. Thus, the nucleic acid may be a synthetic nucleic acid (e.g. a synthetic mRNA). In a further aspect, the invention provides a polypeptide comprising the amino acid sequence of a C. acnes CAMP2 polypeptide. A C. acnes CAMP2 polypeptide for use in the present invention, e.g. delivered as a mRNA and/or in a LNP, may elicit antibodies in a subject. Such antibodies may neutralise biological activity of CAMP2 polypeptide, such as CAMP2 inflammatory activity. Such antibodies may neutralise CAMP2 polypeptide co-hemolytic activity. Neutralisation of CAMP2 inflammatory activity may be determined as reduction in pro-inflammatory cytokines such as IL-1b and/or IL-6. Neutralisation of co-hemolytic activity may be determined in an in vitro co-hemolytic assay.

The amino acid sequence of a native C. acnes CAMP2 antigen is provided in SEQ ID NO: 202:

(SEQ ID NO: 202)

```
                10 20 30 40
MKKTHLVAPL LVGAMLVPAA LSAPSAHAVE PITTISATST
                50 60 70 80
HELSASDARN SIQLLNAHIA TLQSVQKSVP GSDYSDQIRD
                90 100 110 120
LLKAAFDLRG LIETLAHGGI PFYDPSTIMP RIKLVATTID
              130 140 150 160
TIHTATTTLQ NKVRPAHVEL GLEVTKAVLL TANPASTAKE
              170 180 190 200
LDAEGAALKA RLEKVSQYPD LTPNDVATVY VRTNFSKTIW
              210 220 230 240
QVRANRDRYI LGHKSAAVYK TLNHAITKAV GVRLNPKTTV
              250 260
GNIQAARTEL LAAYQTAFNS PDVKKAA
```

The native signal peptide sequence corresponds to residues 1-28 in the amino acid sequence of CAMP2 of C. acnes (SEQ ID NO: 202) and is shown underlined. Residues 29-267 of SEQ ID NO: 202 correspond to the mature form of full-length CAMP2 polypeptide of C. acnes (without the native signal peptide sequence).

The N-terminal domain and C-terminal domain of C. acnes CAMP2 are shown in bold (N terminal domain) and in bold and underlined text (C terminal domain). The N-terminal domain of C. acnes CAMP2 polypeptide corresponds to amino acid residues 29-176 of C. acnes CAMP2 polypeptide in SEQ ID NO: 202. The C-terminal domain of C. acnes CAMP2 polypeptide corresponds to amino acid residues 189-267 of C. acnes CAMP2 polypeptide in SEQ ID NO: 202. A linker domain is positioned between N-terminus and C-terminus domains. The linker domain corresponds to amino acid residues 177-188 of SEQ ID NO: 202.

A “C. acnes CAMP2 polypeptide” includes a mature form of a full-length native CAMP2 polypeptide of C. acnes without its native signal peptide sequence, and immunogenic variants thereof. An immunogenic variant of a native C. acnes CAMP2 polypeptide is capable of eliciting an immune response (e.g. an antigen specific immune response) in a subject, for example an antibody response. Immunogenic variants of a native C. acnes CAMP2 polypeptide include immunogenic fragments of a native C. acnes CAMP2 polypeptide. Immunogenic C. acnes CAMP2 fragments include fragments of a native C. acnes CAMP2 polypeptide that are at least 50, at least 75, at least 100, at least 125, at least 150, at least 175, at least 200 or at least 225 amino acids long.

Exemplary C. acnes CAMP2 polypeptide sequences include a native C. acnes CAMP2 polypeptide sequence in SEQ ID NO: 203 which lacks the native secretion signal peptide sequence of the C. acnes CAMP2 polypeptide. Other exemplary C. acnes polypeptide sequences are set out in SEQ ID NOs 313-338. These are naturally occurring CAMP2 polypeptides from different strains of C. acnes and include the native secretion signal peptide sequence of the C. acnes CAMP2 polypeptide. The corresponding sequences of the CAMP2 polypeptide but without the native secretion signal peptide sequences are provided in SEQ ID NOs 339-363.

Analysis of 430 CAMP2 polypeptide sequences from naturally occurring strains of C. acnes showed that there were only 27 different CAMP2 sequences (SEQ ID NOs 202 and 313-338) when the native secretion signal peptide sequence was included in the alignment and only 26 different CAMP2 sequences when the native secretion signal peptide sequence was excluded (SEQ ID NOs 203 and 339-363). Sequence variation between those CAMP2 polypeptides was concentrated at certain residues (FIGS. 44A-44C and Table 1).

TABLE 1
Analysis of naturally occurring CAMP2 polypeptide variants

| Position | | Number of | % of | | | |
|---|---|---|---|---|---|---|
| within | | sequences | sequences | | | |
| CAMP2 | | identical to | identical to | | Number of | |
| sequence | | KPA171202 | KPA171202 | Residue in | sequences | % of |
| (without | Residue in | (out of 430 | (out of 430 | strains | with variant | sequences |
| signal | strain | sequences | sequences | other than | at that | with variant |
| peptide) | KPA171202* | analysed) | analysed) | KPA171202 | position | sequence |
| 6 | T | 429 | 99.8% | I | 1 | 0.2% |
| 9 | A | 429 | 99.8% | T | 1 | 0.2% |
| 11 | S | 424 | 98.6% | A | 6 | 1.4% |
| 18 | S | 348 | 80.9% | N | 82 | 19.1% |
| 19 | D | 347 | 80.7% | E/Y** | 82/1 | 19%/0.2% |
| 21 | R | 417 | 97.0% | H | 13 | 3.0% |
| 24 | I | 340 | 79.1% | M/L/T | 82/6/2 | 19%/1.4%/0.5% |
| 29 | A | 418 | 97.2% | P | 12 | 2.8% |
| 30 | H | 342 | 79.5% | R | 88 | 20.5% |
| 37 | V | 421 | 97.9% | A | 9 | 2.1% |
| 48 | D | 424 | 98.6% | N | 6 | 1.4% |
| 61 | R | 397 | 92.3% | H | 33 | 7.7% |
| 65 | E | 424 | 98.6% | D | 6 | 1.4% |
| 68 | A | 429 | 99.8% | T | 1 | 0.2% |
| 76 | D | 429 | 99.8% | N | 1 | 0.2% |
| 87 | V | 424 | 98.6% | A | 6 | 1.4% |
| 91 | I | 347 | 80.7% | V | 83 | 19.3% |
| 92 | D | 429 | 99.8% | G | 1 | 0.2% |
| 98 | T | 429 | 99.8% | K | 1 | 0.2% |
| 100 | T | 429 | 99.8% | I | 1 | 0.2% |
| 106 | R | 319 | 74.2% | S | 111 | 25.8% |
| 118 | K | 427 | 99.3% | N | 3 | 0.7% |
| 128 | S | 355 | 82.6% | T | 75 | 17.4% |
| 138 | A | 355 | 82.6% | T | 75 | 17.4% |
| 143 | R | 427 | 99.3% | H | 3 | 0.7% |
| 145 | E | 422 | 98.1% | D/K | 6/2 | 1.4%/0.5% |
| 154 | T | 424 | 98.6% | A | 6 | 1.4% |
| 169 | K | 424 | 98.6% | R | 6 | 1.4% |
| 177 | N | 342 | 79.5% | D | 88 | 20.5% |
| 179 | D | 423 | 98.4% | N/H | 6/16 | 1.4%/3.7% |
| 189 | A | 424 | 98.6% | E | 6 | 1.4% |
| 207 | N | 348 | 80.9% | D/A | 80/2 | 18.6%/0.5% |
| 221 | E | 424 | 98.6% | K | 6 | 1.4% |
| 223 | L | 429 | 99.8% | F | 1 | 23.3% |

*SEQ ID NO: 203 is the CAMP2 sequence of strain KPA171202 without the secretion signal peptide sequence.
**If more than one CAMP2 variant was found at a particular position, all alternative residues are indicated using “/” and their corresponding frequencies are likewise indicated using “/” in the final two columns.

In some embodiments, the C. acnes CAMP2 polypeptide comprises an amino acid substitution at one or more positions corresponding to residues 6, 9, 11, 18, 19, 21, 24, 29, 30, 37, 48, 61, 65, 68, 76, 87, 91, 92, 98, 100, 106, 118, 128, 138, 143, 145, 154, 169, 177, 179, 189, 207, 221 and/or 223 of SEQ ID NO: 203. In some embodiments, the C. acnes CAMP2 polypeptide comprises an amino acid substitution at one or more positions corresponding to residues T6 (e.g. T6I), A9 (e.g. A9T), S11 (e.g. S11A), S18 (e.g. S18N), D19 (e.g. D19E or D19Y), R21 (e.g. R21H), I24 (e.g. I24M, I24L or I24T), A29 (e.g. A29P), H30 (e.g. H30R), V37 (e.g. V37A), D48 (e.g. D48N), R61 (e.g. R61H), E65 (e.g. E65D), A68 (e.g. A68T), D76 (e.g. D76N), V87 (e.g. V87A), 191 (e.g. 191V), D92 (e.g. D92G), T98 (e.g. T98K), T100 (e.g. T100I), R106 (e.g. R106S), K118 (e.g. K118N), S128 (e.g. S128T), A138 (e.g. A138T), R143 (e.g. R143H), E145 (e.g. E145D or E145K), T154 (e.g. T154A), K169 (e.g. K169R), N177 (e.g. N177D), D179 (e.g. D179N or D179H), A189 (e.g. A189E), N207 (e.g. N207D or N207A), E221 (e.g. E221K) and/or L223 (e.g. L223F) of SEQ ID NO: 203.

In some embodiments, the C. acnes CAMP2 polypeptide comprises the sequence of any one of SEQ ID NO: 203, SEQ ID NO: 43-58, SEQ ID NO: 1-4, SEQ ID NO: 10-16, or SEQ ID NO: 339-363 (e.g. SEQ ID NO: 203) or a sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity thereto. Typically, sequence variation is at the positions listed above, i.e. positions corresponding to residues 6, 9, 11, 18, 19, 21, 24, 29, 30, 37, 48, 61, 65, 68, 76, 87, 91, 92, 98, 100, 106, 118, 128, 138, 143, 145, 154, 169, 177, 179, 189, 207, 221 and/or 223 of SEQ ID NO: 203. Thus, in some embodiments, the C. acnes CAMP2 polypeptide comprises an amino acid substitution at one or more positions corresponding to residues 6, 9, 11, 18, 19, 21, 24, 29, 30, 37, 48, 61, 65, 68, 76, 87, 91, 92, 98, 100, 106, 118, 128, 138, 143, 145, 154, 169, 177, 179, 189, 207, 221 and/or 223 of SEQ ID NO: 203. In some embodiments, the C. acnes CAMP2 polypeptide comprises a sequence having at least 90% identity to any one of SEQ ID NO: 203, SEQ ID NO: 43-58, SEQ ID NO: 1-4, SEQ ID NO: 10-16 or SEQ ID NO: 339-363 (e.g. SEQ ID NO: 203). In some embodiments, the C. acnes CAMP2 polypeptide comprises a sequence having at least 95% identity to any one of SEQ ID NO 203, SEQ ID NO: 43-58, SEQ ID NO: 1-4, SEQ ID NO: 10-16 or SEQ ID NO: 339-363 (e.g. SEQ ID NO: 203).

Typically, the C. acnes CAMP2 polypeptide comprises a sequence having at least 85% identity to any one of SEQ ID NO: 203, SEQ ID NO: 43-58, SEQ ID NO: 1-4, SEQ ID NO: 10-16 or SEQ ID NO: 339-363 (e.g. SEQ ID NO: 203). In some embodiments, the C. acnes CAMP2 polypeptide comprises, consists essentially of or consists of (e.g. comprises) an amino acid sequence according to SEQ ID NO: 203.

In some embodiments, a nucleic acid comprising a nucleotide sequence encoding a C. acnes CAMP2 polypeptide comprises a nucleotide sequence according to any one of SEQ ID NO: 153-167, SEQ ID NO: 87-89, or SEQ ID NO: 95-101 or a sequence that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity thereto. In some embodiments, the nucleic acid comprises a nucleotide sequence that is least 75% identical to any one of SEQ ID NO: 153-167, SEQ ID NO: 87-89, or SEQ ID NO: 95-101.

### Modified CAMP2

The inventors have generated modified C. acnes CAMP2 polypeptides comprising a C. acnes CAMP2 polypeptide sequence and a heterologous transmembrane domain (TMB) sequence. The inclusion of a heterologous TMB sequence may be advantageous as such a sequence may localise the antigen to the cell membrane when the antigen is expressed in a cell e.g. in a eukaryotic cell (such as a cell in a subject). The inclusion of a heterologous TMB sequence may reduce antigen intracellular localisation relative to the antigen without the TMB sequence. The inventors have shown that such modified C. acnes CAMP2 polypeptides which comprise a TMB domain sequence elicited an antibody (e.g. IgG) response. Antibodies induced by modified C. acnes CAMP2 polypeptides reduced the co-hemolytic activity of C. acnes CAMP2 polypeptide. C. acnes CAMP2 polypeptides may elicit antibodies in a subject, e.g. antibodies that can neutralise biological activity of CAMP2 polypeptide, such as CAMP2 inflammatory activity.

Moreover, modified C. acnes CAMP2 polypeptides elicited higher antibody titres relative to C. acnes CAMP2 polypeptides lacking a TMB domain. Inclusion of a TMB sequence may therefore enhance immunogenicity (e.g. antibody response) in a subject of a C. acnes CAMP2 polypeptide. Inclusion of a TMB sequence in a modified C. acnes CAMP2 polypeptide may elicit a stronger antibody response, e.g. a higher antibody titre, in a subject compared to a CAMP2 polypeptide without a TMB sequence. A surface-expressed antigen (e.g. a modified C. acnes CAMP2 polypeptide) may enhance B cell activation in a subject leading to enhanced antigen-specific B cell response (e.g. enhanced antigen-specific antibody response such as higher antibody titres) in a subject.

The inventors have therefore demonstrated that these modified C. acnes CAMP2 polypeptides are suitable vaccine antigens that may be used either alone or in combination with other C. acnes antigens described herein, such as one or more of C. acnes DsA1 polypeptides, C. acnes DsA2 polypeptides, C. acnes PITP polypeptides, chimeric C. acnes DsA1/DsA2 polypeptides and chimeric C. acnes DsA1/DsA2/PITP polypeptides described herein. These modified C. acnes CAMP2 polypeptides, when used alone or in combination with one or more other antigens described herein, may elicit an immune response against infection. A modified C. acnes CAMP2 polypeptide as described herein may be delivered by a nucleic acid comprising a nucleotide sequence encoding the modified C. acnes CAMP2 polypeptide.

Accordingly, in one aspect, the invention provides a nucleic acid comprising a nucleotide sequence encoding a modified C. acnes CAMP2 polypeptide, wherein the modified C. acnes CAMP2 polypeptide comprises an amino acid sequence comprising a C. acnes CAMP2 polypeptide sequence and a heterologous transmembrane domain sequence. A heterologous TMB sequence is capable of localising the antigen to the cell membrane when the antigen is expressed in a cell (e.g. a eukaryotic cell such as a cell in a subject). This may enhance immunogenicity (e.g. antibody response such as antibody titre) elicited in a subject, e.g. relative to the antigen lacking the TMB sequence.

In a further aspect, the invention provides a polypeptide comprising the amino acid sequence of a modified C. acnes CAMP2 polypeptide.

In certain embodiments, the heterologous transmembrane domain sequence is a TMB sequence of a eukaryotic transmembrane polypeptide (a eukaryotic transmembrane sequence), a TMB sequence of a prokaryotic transmembrane polypeptide (a prokaryotic transmembrane sequence), or a TMB sequence of a viral transmembrane protein (a viral transmembrane domain sequence). In certain embodiments, the heterologous transmembrane domain is a TMB sequence of a viral transmembrane protein.

In certain embodiments, the heterologous TMB sequence is positioned at the N-terminus of the modified C. acnes CAMP2 polypeptide. In other embodiments, the heterologous TMB sequence is positioned at the C-terminus of the modified C. acnes CAMP2 polypeptide.

Typically, a nucleic acid described herein encoding the modified C. acnes CAMP2 polypeptide that comprises a transmembrane domain also comprises a nucleotide sequence encoding a secretion signal peptide sequence.

Additional detail regarding TMB domains suitable for use in the present invention is provided herein below. Exemplary TMB sequences are described herein below.

In certain embodiments, the heterologous transmembrane domain comprises a hemagglutinin (HA) transmembrane domain sequence from influenza A or influenza B virus, preferably from influenza A virus.

In certain embodiments, a transmembrane domain (TMB) sequence is directly fused to a C. acnes CAMP2 polypeptide described herein (i.e., there is no linker, such as an amino acid linker, connecting the TMB sequence to the C. acnes CAMP2 polypeptide in the modified C. acnes CAMP2 polypeptide described herein). In other embodiments of the modified C. acnes CAMP2 polypeptide described herein, a TMB sequence of the disclosure is attached to a C. acnes CAMP2 polypeptide described herein with a linker. Preferred linkers are set out herein below in section “Linkers”.

In some embodiments, the modified C. acnes CAMP2 polypeptide comprises a C. acnes CAMP2 polypeptide sequence which comprises a sequence according to any one of SEQ ID NO: 203, SEQ ID NO: 43-58, SEQ ID NO: 1-4, SEQ ID NO: 10-16, or SEQ ID NO: 339-363 (e.g. SEQ ID NO: 203) or a sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity thereto. Typically, variation in the C. acnes CAMP2 polypeptide sequence may be at the positions set out herein above. Thus, C. acnes CAMP2 polypeptide sequence variation may typically be at positions corresponding to residues 6, 9, 11, 18, 19, 21, 24, 29, 30, 37, 48, 61, 65, 68, 76, 87, 91, 92, 98, 100, 106, 118, 128, 138, 143, 145, 154, 169, 177, 179, 189, 207, 221 and/or 223 of SEQ ID NO: 203 as set out herein above. In some embodiments, the C. acnes CAMP2 polypeptide sequence comprises an amino acid substitution at one or more positions corresponding to residues 6, 9, 11, 18, 19, 21, 24, 29, 30, 37, 48, 61, 65, 68, 76, 87, 91, 92, 98, 100, 106, 118, 128, 138, 143, 145, 154, 169, 177, 179, 189, 207, 221 and/or 223 of SEQ ID NO: 203. In some embodiments, the C. acnes CAMP2 polypeptide comprises an amino acid substitution at one or more positions corresponding to residues T6 (e.g. T6I), A9 (e.g. A9T), S11 (e.g. S11A), S18 (e.g. S18N), D19 (e.g. D19E or D19Y), R21 (e.g. R21H), I24 (e.g. I24M, I24L or I24T), A29 (e.g. A29P), H30 (e.g. H30R), V37 (e.g. V37A), D48 (e.g. D48N), R61 (e.g. R61H), E65 (e.g. E65D), A68 (e.g. A68T), D76 (e.g. D76N), V87 (e.g. V87A), 191 (e.g. 191V), D92 (e.g. D92G), T98 (e.g. T98K), T100 (e.g. T100I), R106 (e.g. R106S), K118 (e.g. K118N), S128 (e.g. S128T), A138 (e.g. A138T), R143 (e.g. R143H), E145 (e.g. E145 D or E145K), T154 (e.g. T154A), K169 (e.g. K169R), N177 (e.g. N177D), D179 (e.g. D179N or D179H), A189 (e.g. A189E), N207 (e.g. N207D or N207A), E221 (e.g. E221K) and/or L223 (e.g. L223F) of SEQ ID NO: 203. In some embodiments, the modified C. acnes CAMP2 polypeptide comprises a C. acnes CAMP2 polypeptide sequence which comprises a sequence having at least 90% identity to any one of SEQ ID NO 203, SEQ ID NO: 43-58, SEQ ID NO: 1-4, SEQ ID NO: 10-16 or SEQ ID NO: 339-363 (e.g. SEQ ID NO: 203). In some embodiments, the modified C. acnes CAMP2 polypeptide comprises a C. acnes CAMP2 polypeptide sequence which comprises a sequence having at least 95% identity to any one of SEQ ID NO 203, SEQ ID NO: 43-58, SEQ ID NO: 1-4, SEQ ID NO: 10-16 or SEQ ID NO: 339-363 (e.g. SEQ ID NO: 203). Typically, the modified C. acnes CAMP2 polypeptide comprises a C. acnes CAMP2 polypeptide sequence which comprises a sequence having at least 85% identity to any one of SEQ ID NO: 203, SEQ ID NO: 43-58, SEQ ID NO: 1-4, SEQ ID NO: 10-16 or SEQ ID NO: 339-363 (e.g. SEQ ID NO: 203).

In some embodiments, the modified C. acnes CAMP2 polypeptide comprises a sequence according to any one of SEQ ID NO: 207 or SEQ ID NO: 5-9 (e.g. SEQ ID NO: 207) or a sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity thereto. Typically, sequence variation is at the positions set out herein above, i.e. positions corresponding to residues 6, 9, 11, 18, 19, 21, 24, 29, 30, 37, 48, 61, 65, 68, 76, 87, 91, 92, 98, 100, 106, 118, 128, 138, 143, 145, 154, 169, 177, 179, 189, 207, 221 and/or 223 of SEQ ID NO: 207. Thus, in some embodiments, the modified C. acnes CAMP2 polypeptide comprises an amino acid substitution at one or more positions corresponding to residues 6, 9, 11, 18, 19, 21, 24, 29, 30, 37, 48, 61, 65, 68, 76, 87, 91, 92, 98, 100, 106, 118, 128, 138, 143, 145, 154, 169, 177, 179, 189, 207, 221 and/or 223 relative to the residue numbering in SEQ ID NO: 207. In some embodiments, the modified C. acnes CAMP2 polypeptide comprises a sequence having at least 90% identity to any one of SEQ ID NO: 203, SEQ ID NO: 207, SEQ ID NO: 43-58, SEQ ID NO: 1-16 or SEQ ID NO: 339-363 (e.g., SEQ ID NO: 207). In some embodiments, the modified C. acnes CAMP2 polypeptide comprises a sequence having at least 95% identity to any one of SEQ ID NO: 203, SEQ ID NO: 207, SEQ ID NO: 43-58, SEQ ID NO: 1-16 or SEQ ID NO: 339-363 (e.g., SEQ ID NO: 207). In some embodiments, the modified C. acnes CAMP2 polypeptide comprises a sequence having at least 85% identity to any one of SEQ ID NO: 203, SEQ ID NO: 207, SEQ ID NO: 43-58, SEQ ID NO: 1-16 or SEQ ID NO: 339-363 (e.g., SEQ ID NO: 207).

In some embodiments, a nucleic acid comprising a nucleotide sequence encoding a modified C. acnes CAMP2 polypeptide comprises a nucleotide sequence according to any one of SEQ ID NO: 153-167, SEQ ID NO: 87-89 or SEQ ID NO: 95-101 or a sequence that has at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity thereto. In some embodiments, a nucleic acid comprising a nucleotide sequence encoding a modified C. acnes CAMP2 polypeptide comprises a nucleotide sequence according to any one of SEQ ID NO: 90-94 or SEQ ID NO: 391-392 (e.g., SEQ ID NO: 91) or a sequence that has at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity thereto. In some embodiments, the nucleic acid comprises a nucleotide sequence that is at least 75% or 85% (e.g., 75%) identical to any one of SEQ ID NO: 153-167, SEQ ID NO: 391-392 or SEQ ID NO: 87-101 (e.g., SEQ ID NO: 91).

The sequences of SEQ ID NOs: 90, 91, 391 and 392 were aligned. Variation was found at the positions indicated in SEQ ID NOs 398 and 399. In some embodiments, the nucleic acid comprising a nucleotide sequence encoding a modified C. acnes CAMP2 polypeptide comprises a nucleotide sequence according to SEQ ID NO: 398, wherein the nucleotides at positions 66; 78; 81; 87; 102; 120; 150; 222; 232; 234; 261; 298; 321; 339; 351; 367; 420; 423; 435; 438; 456; 462; 468; 478; 516; 528; 541; 546; 574; 583; 648; 664; 684; 706; 711; 735; 834 are each independently selected from A or C; the nucleotides at positions 480; 660; 849 are each independently selected from A, C or G; the nucleotides at positions 171; 282; 318; 375; 432; 675; 738 are each independently selected from A, C or T; the nucleotides at positions 6; 57; 93; 114; 168; 246; 300; 363; 384; 396; 405; 447; 459; 543; 585; 591; 609; 666; 708; 714; 857 are each independently selected from A or G; the nucleotides at positions 73; 97; 103; 118; 169; 181; 190; 283; 433; 493; 553; 745; 769; 775; 793; 805; 808; 826; 841; 850 are each independently selected from A or T; the nucleotides at positions 18; 27; 54; 98; 119; 153; 162; 170; 182; 191; 210; 213; 240; 264; 284; 312; 369; 390; 408; 434; 441; 483; 494; 554; 573; 603; 690; 746; 770; 774; 776; 794; 806; 809; 827; 842; 851; 855 are each independently selected from C or G; and the nucleotides at positions 30; 33; 36; 48; 51; 108; 111; 117; 123; 135; 177; 183; 186; 189; 207; 219; 225; 243; 255; 258; 267; 270; 276; 279; 285; 297; 327; 372; 378; 402; 426; 429; 448; 453; 465; 477; 501; 507; 519; 522; 531; 537; 549; 564; 582; 594; 615; 624; 636; 672; 693; 696; 702; 729; 741; 750; 753; 771; 780; 795; 804; 807; 810; 828; 837; 840; 852 are each independently selected from C or T. In some embodiments, the nucleic acid comprising a nucleotide sequence encoding a modified C. acnes CAMP2 polypeptide comprises a nucleotide sequence according to SEQ ID NO: 399, wherein the nucleotides at positions 66; 81; 87; 102; 222; 232; 234; 261; 298; 321; 339; 351; 420; 438; 456; 462; 468; 478; 516; 528; 541; 546; 574; 583; 648; 660; 664; 684; 706; 711; 735; 834 are each independently selected from A or C; the nucleotide at position 849 is selected from A, C or G; the nucleotides at positions 318; 432; 675 are each independently selected from A, C or T; the nucleotides at positions 6; 57; 93; 168; 300; 363; 384; 396; 405; 447; 459; 480; 543; 585; 609; 666; 708; 714; 857 are each independently selected from A or G; the nucleotides at positions 73; 97; 103; 118; 169; 181; 283; 433; 493; 745; 769; 793; 805; 808; 826; 841; 850 are each independently selected from A or T; the nucleotides at positions 18; 27; 54; 98; 119; 153; 170; 182; 213; 264; 284; 312; 369; 390; 408; 434; 483; 494; 573; 603; 746; 770; 774; 794; 806; 809; 827; 842; 851; 855 are each independently selected from C or G; and the nucleotides at positions 30; 33; 36; 48; 117; 123; 135; 171; 177; 183; 186; 189; 225; 243; 255; 258; 267; 270; 276; 282; 285; 297; 327; 372; 375; 378; 426; 453; 465; 477; 501; 519; 522; 531; 549; 564; 582; 624; 636; 672; 693; 696; 738; 741; 750; 753; 771; 780; 795; 807; 810; 828; 837; 840; 852 are each independently selected from C or T. Typically, the nucleic acid comprising a nucleotide sequence according to SEQ ID NO: 398 or 399 encodes a sequence according to SEQ ID NO: 6.

In some embodiments, the nucleic acid comprises a nucleotide sequence that is at least 75% or 85% (e.g., 75%) identical to any one of SEQ ID NO: 153-167, SEQ ID NO: 391-392 or SEQ ID NO: 87-101 (e.g., SEQ ID NO: 91).

In one embodiment, the nucleic acid of the invention is a mRNA comprising or consisting of (e.g., consisting of) the following structural elements:
(i) a 5′ cap with the following structure:

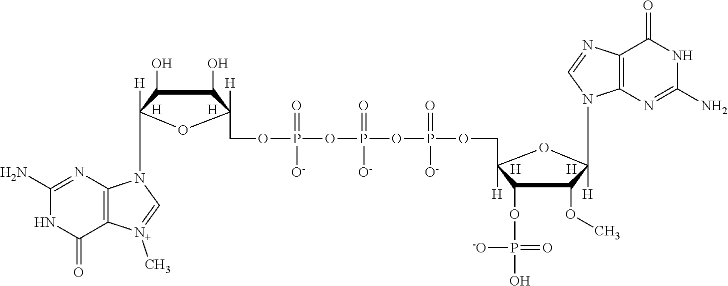

(ii) a 5′ untranslated region (5′ UTR) having the nucleic acid sequence according to SEQ ID NO: 265;
(iii) a protein coding region having the nucleic acid sequence according to SEQ ID NO: 90, SEQ ID NO: 391-392 or SEQ ID NO: 91 (e.g., SEQ ID NO: 91);
(iv) a 3′ untranslated region (3′ UTR) having the nucleic acid sequence according to SEQ ID NO: 266; and
(v) a polyA tail, optionally wherein the polyA tail comprises at least 75 adenosine nucleotides (such as about 80 adenosine nucleotides) or at least 100 adenosine nucleotides (such as about 115 adenosine nucleotides), e.g., wherein the polyA tail comprises at least 100 adenosine nucleotides.

In some embodiments, the mRNA is chemically modified and the chemical modification comprises N1-methylpseudouridine in place of every uridine. The mRNA may be encapsulated in a LNP.

### DsA1

C. acnes dermatan sulfate-adhesin 1 (DsA1), also known as P22 or P022, was identified as a putative C. acnes virulence factor (Lodes et al, 2006 (Microbiology (Reading). 2006 December; 152 (Pt 12): 3667-3681); McDowell et al., 2011). This protein is found in abundance in both acne-affected and healthy follicular samples (Bek-Thomsen et al., 2014). C. acnes DsA1 polypeptides, derivatives and fragments thereof are described in WO2021/165543.

The inventors have demonstrated that mRNAs encoding different C. acnes DsA1 polypeptides and recombinant C. acnes DsA1 proteins elicit an antibody (e.g. IgG) response. Antibodies induced by C. acnes DsA1 polypeptides were shown to bind to the surface of C. acnes bacteria and recruit effector cells (e.g. effector cells of the immune system such as phagocytes). Antibodies induced by C. acnes DsA1 polypeptides were shown to elicit opsonophagocytic killing in vitro.

C. acnes DsA1 polypeptides of the invention may induce an immune response against a broad range of different C. acnes strains, phylotypes and variants which cause disease (a cross-reactive immune response).

The inventors have demonstrated that C. acnes DsA1 polypeptides are suitable vaccine antigens that may be used in combination with one or more other C. acnes antigens that can elicit antibody responses in a subject, such as C. acnes DsA2 polypeptides described herein, C. acnes PITP polypeptides described herein, and C. acnes CAMP2 polypeptides described herein. C. acnes CAMP2 polypeptides may elicit antibodies in a subject, e.g., antibodies that can neutralise biological activity of CAMP2 polypeptide, such as CAMP2 inflammatory activity.

Accordingly, in one aspect, the invention provides a nucleic acid that comprises a nucleotide sequence encoding a C. acnes DsA1 polypeptide. In a further aspect, the invention provides a polypeptide comprising the amino acid sequence of a C. acnes DsA1 polypeptide. A C. acnes DsA1 polypeptide for use in the present invention, e.g. delivered as a mRNA or as a recombinant protein, may elicit antibodies in a subject. Such antibodies may opsonize C. acnes bacteria. Opsonization may target immune effector cells such as phagocytes to C. acnes bacteria. This may lead to killing of C. acnes bacteria by such immune effector cells (e.g., phagocytic killing). Antibody binding to C. acnes cell surface may be measured e.g., using in vitro surface binding assays. In vitro opsonophagocytic killing assays may be used to evaluate antibodies for their ability to induce opsonization and killing of C. acnes strains of different genetic types by phagocytic cells.

The amino acid sequence of a native mature form of full-length C. acnes DsA1 polypeptide (without a signal peptide sequence) is provided in SEQ ID NO: 204:

SSNRPRSVAQAAIATDGKGIIDKDCRDAVINDAKLRAAIAGALVKAGFSS
ADAVALAPRIAKEMAKEGVLLINHHKLKALIGAQLGLLTDAKIQRAAAAV
DLGIKATLAATIIPNALHSAAFKDAVVANLVAAGVDKKLAKATAVAIAAT
ALNPALGPIAKTEAIKAEIAAQAALLVGRGVHLKKAAIEHIIGRSFDAAV
ATAIVSSPILNARIVTHLVRAGIDKSLAVQIAPRIIDRLAKEPLLALNTA
KLMKNITRQIVDVITADKAIKTAEQLEKELPALDDLVKKACSCPKPTPTP
TPTPTPTPKPTPTPTPKPTPTPKPKPTPAPAPTSGATSDESTSRSGGHSQ
GGSGTHYIHHGVAPVLTHSSDLPSTGF

A native mature C. acnes DsA1 polypeptide typically comprises a N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”), a Pro-Thr repeat containing region (“PT repeat region”-shown in bold above), and a C-terminal region (“CTR”; often with an LPXTG motif close to the C-terminus). The C-terminal LPXTG motif is thought to be critical for cell-wall anchoring of the protein.

For example, in the native C. acnes DsA1 polypeptide of SEQ ID NO: 204, the N-terminal swapping region (“NSR”) corresponds to amino acid residues 1-20 the first conserved sub-domain (“CSD1”) corresponds to amino acid residues 21-102, the first swapping region (“SR1”) corresponds to amino acid residues 103-119, the second conserved sub-domain (“CSD2”) corresponds to amino acid residues 120-239, the second swapping region (“SR2”) corresponds to amino acid residues 240-249, the third conserved sub-domain (“CSD3”) corresponds to amino acid residues 250-295, the Pro-Thr repeat containing region (“PT repeat region”) corresponds to amino acid residues 296-333, and the C-terminal region (“CTR”) corresponds to amino acid residues 334-377 (with the LPXTG motif corresponding to amino acid residues 372-376).

The sequence of naturally occurring DsA1 polypeptides are for the most part highly identical and differ (besides point mutations and rare exceptions specifically of the presence of the terminal LPXTG motif) only in the length and composition of the PT repeat region. More detailed sequence analyses of DsA1 have been described elsewhere (WO2021/165543). As is known in the art, the positions of the NSR, CSD1, SRI, CSD2, SR2, CSD3, PT repeat region and CTR within other native C. acnes DsA1 polypeptides may be determined by aligning the polypeptide sequence of a given DsA1 polypeptide with that of SEQ ID NO: 204, and identifying the regions of the DsA1 polypeptide which have high sequence identity to that of the NSR, CSD1, SRI, CSD2, SR2, CSD3, PT repeat region and CTR of SEQ ID NO: 204, respectively.

A “C. acnes DsA1 polypeptide” includes a mature form of a full-length native DsA1 polypeptide of C. acnes without its native signal peptide sequence, and immunogenic variants thereof. An immunogenic variant of a native C. acnes DsA1 polypeptide is capable of eliciting an immune response (e.g. an antigen specific immune response) in a subject, for example an antibody response. Immunogenic variants of a native C. acnes DsA1 polypeptide include immunogenic fragments of a native C. acnes DsA1 polypeptide. Immunogenic C. acnes DsA1 fragments include fragments of a native C. acnes DsA1 polypeptide that are at least 50, at least 75, at least 100, at least 125, at least 150, at least 175, at least 200, at least 225, at least 250, at least 275, at least 300, at least 325 or at least 350 amino acids long. In some embodiments, an immunogenic C. acnes DsA1 fragment comprises a CSD2 domain of a C. acnes DsA1 polypeptide. In some embodiments, immunogenic variants exclude sequence motifs that are found in a subject (e.g. human) proteome, e.g. sequence motifs of 8 or more, 9 or more, 10 or more, 11 or more, 12 or more, 13 or more, 14 or more or 15 or more (e.g. 8 or more) amino acids in length. Excluded subject proteome sequence motifs are typically 8 or more amino acids in length. This helps minimise unwanted cross-reactivity due to homology between the antigen and self protein.

Exemplary C. acnes DsA1 polypeptide sequences include a native C. acnes DsA1 polypeptide sequence of SEQ ID NO: 204 which lacks the native secretion signal peptide sequence of the C. acnes DsA1 polypeptide.

In some embodiments, the C. acnes DsA1 polypeptide comprises the sequence of any one of SEQ ID NO: 204, SEQ ID NO: 17-19 or SEQ ID NO: 59-61 or a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity thereto. In some embodiments, the C. acnes DsA1 polypeptide comprises a sequence having at least 90% identity to any one of SEQ ID NO: 204, SEQ ID NO: 17-19 or SEQ ID NO: 59-61. In some embodiments, the C. acnes DsA1 polypeptide comprises a sequence having at least 95% identity to any one of SEQ ID NO: 204, SEQ ID NO: 17-19 or SEQ ID NO: 59-61.

In some embodiments, a nucleic acid comprising a nucleotide sequence encoding a C. acnes DsA1 polypeptide comprises a nucleotide sequence according to any one of SEQ ID NO: 102-104 or SEQ ID NO: 168-170 or a sequence that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity thereto. In some embodiments, the nucleic acid comprises a nucleotide sequence that is least 75% identical to any one of SEQ ID NO: 102-104 or SEQ ID NO: 168-170.

### DsA2

C. acnes dermatan sulfate-adhesin 2 (DsA2) is also known as P27 or P027. DsA2 was identified as a putative C. acnes virulence factor (Lodes et al, 2006 (Microbiology (Reading). 2006 December; 152 (Pt 12): 3667-3681); McDowell et al., 2011). This protein is found in both acne-affected and healthy follicular samples (Bek-Thomsen et al., 2014). DsA1 and DsA2 proteins are homologues (paralogues) with a typical sequence identity between 60-71%, depending on which region of the protein is aligned. Excluding PT-region length polymorphism, a high degree of typically >90% sequence identity can be seen within intact DsA1 and DsA2 proteins, respectively. C. acnes DsA2 polypeptides, derivatives and fragments thereof are described in WO2021/165543.

The inventors have demonstrated that mRNAs encoding different C. acnes DsA2 polypeptides and recombinant C. acnes DsA2 proteins elicit an antibody (e.g. IgG) response. Antibodies induced by C. acnes DsA2 polypeptides were shown to bind to the surface of C. acnes bacteria and recruit effector cells (e.g. effector cells of the immune system such as phagocytes). Antibodies induced by C. acnes DsA2 polypeptides were shown to elicit opsonophagocytic killing in vitro.

C. acnes DsA2 polypeptides of the invention may induce an immune response against a broad range of different C. acnes strains, phylotypes and variants which cause disease (a cross-reactive immune response).

The inventors have demonstrated that C. acnes DsA2 polypeptides are suitable vaccine antigens that may be used in combination with one or more other C. acnes antigens that can elicit antibody responses in a subject, such as C. acnes DsA1 polypeptides described herein, C. acnes PITP polypeptides described herein, and C. acnes CAMP2 polypeptides described herein. C. acnes CAMP2 polypeptides may elicit antibodies in a subject, e.g., antibodies that can neutralise biological activity of CAMP2 polypeptide, such as CAMP2 inflammatory activity.

Accordingly, in one aspect, the invention provides a nucleic acid that comprises a nucleotide sequence encoding a C. acnes DsA2 polypeptide. In a further aspect, the invention provides a polypeptide comprising the amino acid sequence of a C. acnes DsA2 polypeptide. A C. acnes DsA2 polypeptide for use in the present invention, e.g. delivered as a mRNA or as a recombinant protein, may elicit antibodies in a subject. Such antibodies may opsonize C. acnes bacteria. Opsonization may target immune effector cells such as phagocytes to C. acnes bacteria. This may lead to killing of C. acnes bacteria by such immune effector cells (e.g., phagocytic killing). Antibody binding to C. acnes cell surface may be measured e.g, using in vitro surface binding assays. In vitro opsonophagocytic killing assays may be used to evaluate antibodies for their ability to induce opsonization and killing of C. acnes strains of different genetic types by phagocytic cells.

The amino acid sequence of a native mature form of full-length C. acnes DsA2 polypeptide (without a signal peptide sequence) is provided in SEQ ID NO: 205:

ASNGNSSITQSAAFSPRATTKISEDCRKAIINDLKLRGAIVGALVKAGLS
AADAAALAPRIAAEMAAEGTLTINHHRLKVLVASQLGLVADAAVQHAAAA
IDLSFKAILGASIIPNALGSAAFKNAVIANLVAAGIDKHLARATAVAIVA
TALNPALGPIAKFELIKAEIAAQAALLIRRGVHLQKAAIEHVIGRAFDAA
VATAIISSPILSARIVTHLVRAGIDKSIAISLAPHIVKRLAKEPLLAFNT
AKLVKDIARQIVDIRNTQEAIAVYKQLKAELPTLDGLVQKACTPEPTPTP
TPTPTPTPTPAPTPTPAPTPTPAPTPAPTPTPAPTPTPTPTPTPTPTPTH
GATTTTPISRTTDRHNLGSHHTRIAAPALIHAKALPATGTGA

A native mature C. acnes DsA2 polypeptide typically comprises a N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”), a Pro-Thr repeat containing region (“PT repeat region”), and a C-terminal region (“CTR”; often with an LPXTG motif close to the C-terminus). The C-terminal LPXTG motif is thought to be critical for cell-wall anchoring of the protein.

For example, in the native C. acnes DsA2 polypeptide of SEQ ID NO: 205, the N-terminal swapping region (“NSR”) corresponds to amino acid residues 1-21, the first conserved sub-domain (“CSD1”) corresponds to amino acid residues 22-103, the first swapping region (“SR1”) corresponds to amino acid residues 104-120, the second conserved sub-domain (“CSD2”) corresponds to amino acid residues 121-240, the second swapping region (“SR2”) corresponds to amino acid residues 241-250, the third conserved sub-domain (“CSD3”) corresponds to amino acid residues 251-295, the Pro-Thr repeat containing region (“PT repeat region”) corresponds to amino acid residues 296-349, and the C-terminal region (“CTR”) corresponds to amino acid residues 350-392 (with the LPXTG motif corresponding to amino acid residues 385-389).

The sequence of naturally occurring DsA2 polypeptides are for the most part highly identical and differ (besides point mutations and rare exceptions specifically of the presence of the terminal LPXTG motif) only in the length and composition of the PT repeat region. More detailed sequence analyses of DsA2 have been described elsewhere (WO2021/165543). As is known in the art, the positions of the NSR, CSD1, SR1, CSD2, SR2, CSD3, PT repeat region and CTR within other native C. acnes DsA2 polypeptides may be determined by aligning the polypeptide sequence of a given DsA2 polypeptide with that of SEQ ID NO: 205, and identifying the regions of the DsA2 polypeptide which have high sequence identity to that of the NSR, CSD1, SR1, CSD2, SR2, CSD3, PT repeat region and CTR of SEQ ID NO: 205, respectively.

A “C. acnes DsA2 polypeptide” includes a mature form of a full-length native DsA2 polypeptide of C. acnes without its native signal peptide sequence, and immunogenic variants thereof. An immunogenic variant of a native C. acnes DsA2 polypeptide is capable of eliciting an immune response (e.g. an antigen specific immune response) in a subject, for example an antibody response. Immunogenic variants of a native C. acnes DsA2 polypeptide include immunogenic fragments of a native C. acnes DsA2 polypeptide. Immunogenic C. acnes DsA2 fragments include fragments of a native C. acnes DsA2 polypeptide that are at least 50, at least 75, at least 100, at least 125, at least 150, at least 175, at least 200, at least 225, at least 250, at least 275, at least 300, at least 325, at least 350 or at least 375 amino acids long. In some embodiments, an immunogenic C. acnes DsA2 fragment comprises a CSD2 domain of a C. acnes DsA2 polypeptide. In some embodiments, immunogenic variants exclude sequence motifs that are found in a subject (e.g. human) proteome, e.g. sequence motifs of 8 or more, 9 or more, 10 or more, 11 or more, 12 or more, 13 or more, 14 or more or 15 or more (e.g. 8 or more) amino acids in length. Excluded subject proteome sequence motifs are typically 8 or more amino acids in length. As explained further above, this helps minimise unwanted cross-reactivity due to homology between the antigen and self protein.

Exemplary C. acnes DsA2 polypeptide sequences include a native C. acnes DsA2 polypeptide sequence in SEQ ID NO: 205 which lacks the native secretion signal peptide sequence of the C. acnes DsA2 polypeptide.

In some embodiments, the C. acnes DsA2 polypeptide comprises the sequence of any one of SEQ ID NO: 205, SEQ ID NO: 20-27 or SEQ ID NO: 62-69 or a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity thereto. In some embodiments, the C. acnes DsA2 polypeptide comprises a sequence having at least 90% identity to any one of SEQ ID NO: 205, SEQ ID NO: 20-27 or SEQ ID NO: 62-69. In some embodiments, the C. acnes DsA2 polypeptide comprises a sequence having at least 95% identity to any one of SEQ ID NO: 205, SEQ ID NO: 20-27 or SEQ ID NO: 62-69.

In some embodiments, a nucleic acid comprising a nucleotide sequence encoding a C. acnes DsA2 polypeptide comprises a nucleotide sequence according to any one of SEQ ID NO: 105-112 or SEQ ID NO: 171-178 or a sequence that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity thereto. In some embodiments, the nucleic acid comprises a nucleotide sequence that is least 75% identical to any one of SEQ ID NO: 105-112 or SEQ ID NO: 171-178.

### PITP

C. acnes putative iron-transport protein (PITP), also known as P28 or P028, is a protein involved in the iron uptake mechanism by C. acnes (Lodes et al., 2006). Iron uptake plays a role in bacterial survival in the host tissue. Under iron limiting conditions, the expression of PITP increases on the cell surface (Lodes et al., 2006). C. acnes PITP polypeptides, derivatives and fragments thereof are described in WO2021/165543.

C. acnes PITP polypeptides of the invention may induce an immune response against a broad range of different C. acnes strains, phylotypes and variants which are able to cause disease. C. acnes PITP polypeptides were shown to elicit antibodies that bind to the surface of C. acnes bacteria and recruit effector cells (e.g. effector cells of the innate immune system such as phagocytes). The combination of a C. acnes PITP polypeptide of the invention with C. acnes DsA1 and/or DsA2 polypeptides (delivered as a mRNA or in the form of a recombinant protein) may elicit an immune response against a greater number of C. acnes strains or phylotypes compared to use of individual antigens.

The inventors have demonstrated that mRNAs encoding different C. acnes PITP polypeptides and recombinant C. acnes PITP proteins elicit an antibody (e.g. IgG) response. Antibodies elicited by C. acnes PITP polypeptides were shown to bind to the surface of C. acnes bacteria. Antibodies elicited by C. acnes PITP polypeptides were shown to elicit opsonophagocytic killing in vitro. Antibodies elicited by C. acnes PITP polypeptides were also shown to be cross-reactive across a range of C. acnes strains.

The inventors have demonstrated that C. acnes PITP polypeptides are suitable vaccine antigens that may be used in combination with one or more other C. acnes antigens that can elicit antibody responses in a subject, such as C. acnes DsA1 polypeptides described herein, C. acnes DsA2 polypeptides described herein, and C. acnes CAMP2 polypeptides described herein. C. acnes CAMP2 polypeptides may elicit antibodies in a subject, e.g. antibodies that can neutralise biological activity of CAMP2 polypeptide, such as CAMP2 inflammatory activity.

Accordingly, in one aspect, the invention provides a nucleic acid that comprises a nucleotide sequence encoding a C. acnes PITP polypeptide. In a further aspect, the invention provides a polypeptide comprising the amino acid sequence of a C. acnes PITP polypeptide. A C. acnes PITP polypeptide for use in the present invention, e.g. delivered as a mRNA or as a recombinant protein, may elicit antibodies in a subject. Such antibodies may opsonize C. acnes bacteria. Opsonization may target immune effector cells such as phagocytes to C. acnes bacteria. This may lead to killing of C. acnes bacteria by such immune effector cells (e.g. phagocytic killing). Antibody binding to C. acnes cell surface may be measured e.g. using in vitro surface binding assays. In vitro opsonophagocytic killing assays may be used to evaluate antibodies for their ability to induce opsonization and killing of C. acnes strains of different genetic types by phagocytic cells.

The amino acid sequence of a native mature form of full-length C. acnes PITP polypeptide (without a signal peptide sequence) is provided in SEQ ID NO: 206:

AGPTVTVIPVGREGGDITISGKGFSTTGFGVYVAVAPASVPEFYGNSDKF
YGYDPSKDTTESPSTIWVYTPSQKAIGSRFAQGRPMNNDGSFTITMKAPP
FEQGKDFVVLTTKAHGVGKTDHSDDTRTPVTYREATPAPTGPKTPIAPSK
QPSKQAAPSKQVKPSKQAGPNKQSTTPQQKTAEHRSQTPAAHRTMTKQVC
TIGASKVTSGSLTWGIRTSFTSYLRGPIANGSWKLSGGANWNGSAFTFPL
TSGSFDPATKSGSLKYSGSVHMTGHHGILDMTLAEPSLQIKGSTGHLYLD
VKSSSMDGKKTNYGRVDFATFGVSVSGNAAIKGSPVKLTATGAKAFAGFY
RAGEPMNPLSTNLTLSAEKVCHNVTVDAVTGKVIGDDSGKGAGRGLPVTG
AEGPSSDEIDLGIVGGLALTAVVSTVVVCRRYAARI

A native, mature C. acnes PITP polypeptide typically comprises an extended neocarzinostatin family domain (“ENFD”), a first swapping region (“SR1”), a heme binding domain (“HbD”), a second swapping region (“SR2”; which includes the first four N-terminal residues of a LPXTG motif (i.e. including LPXT, but not G)), and a hydrophobic C-terminal region (“hLAR”; which includes the C-terminal Gly residue of a LPXTG motif). The LPXTG motif is thought to be critical for cell-wall anchoring of the protein.

For example, in the native C. acnes PITP polypeptide of SEQ ID NO: 206, the extended neocarzinostatin family domain (“ENFD”) corresponds to amino acid residues 1-133, the first swapping region (“SR1”) corresponds to amino acid residues 134-206, the heme binding domain (“HbD”) corresponds to amino acid residues 207-365, the second swapping region (“SR2”) corresponds to amino acid residues 366-399, and the hydrophobic C-terminal region (“hLAR”) corresponds to amino acid residues 400-436. The LPXTG motif corresponds to amino acid residues 396-400.

The sequence of naturally occurring PITP polypeptides are for the most part highly identical and differ (besides point mutations) only in the N- and in the C-terminus. More detailed sequence analyses of DsA1 have been described elsewhere (WO2021/165543). As is known in the art, the positions of the ENFD, SR1, HbD, SR2 and hLAR within other native C. acnes PITP polypeptides may be determined by aligning the polypeptide sequence of a given PITP polypeptide with that of SEQ ID NO: 206, and identifying the regions of the PITP polypeptide which have high sequence identity to that of the ENFD, SR1, HbD, SR2 and hLAR of SEQ ID NO: 206, respectively.

A “C. acnes PITP polypeptide” includes a mature form of a full-length native PITP polypeptide of C. acnes without its native signal peptide sequence, and immunogenic variants thereof. An immunogenic variant of a native C. acnes PITP polypeptide is capable of eliciting an immune response (e.g. an antigen specific immune response) in a subject, for example an antibody response. Immunogenic variants of a native C. acnes PITP polypeptide include immunogenic fragments of a native C. acnes PITP polypeptide. Immunogenic C. acnes PITP fragments include fragments of a native C. acnes PITP polypeptide that are at least 50, at least 75, at least 100, at least 125, at least 150, at least 175, at least 200, at least 225, at least 250, at least 275, at least 300, at least 325, at least 350, at least 375, at least 400 or at least 425 amino acids long. In some embodiments, an immunogenic C. acnes PITP fragment comprises a ENFD and/or a HbD (e.g. a ENFD) domain of a C. acnes PITP polypeptide. In some embodiments, immunogenic variants exclude sequence motifs that are found in a subject (e.g. human) proteome, e.g. sequence motifs of 8 or more, 9 or more, 10 or more, 11 or more, 12 or more, 13 or more, 14 or more or 15 or more (e.g. 8 or more) amino acids in length. Excluded subject proteome sequence motifs are typically 8 or more amino acids in length. As explained further above, this helps minimise unwanted cross-reactivity due to homology between the antigen and self protein.

Exemplary C. acnes PITP polypeptide sequences include a native C. acnes PITP polypeptide sequence in SEQ ID NO: 206 which lacks the native secretion signal peptide sequence of the C. acnes PITP polypeptide.

In some embodiments, the C. acnes PITP polypeptide comprises the sequence of any one of SEQ ID NO: 206, SEQ ID NO: 31-37, SEQ ID NO: 73-79 or a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity thereto. In some embodiments, the C. acnes PITP polypeptide comprises a sequence having at least 90% identity to any one of SEQ ID NO 206, SEQ ID NO: 31-37 or SEQ ID NO: 73-79. In some embodiments, the C. acnes PITP polypeptide comprises a sequence having at least 95% identity to any one of SEQ ID NO: 206, SEQ ID NO: 31-37 or SEQ ID NO: 73-79. In some embodiments, the C. acnes PITP polypeptide comprises, consists of or consists essentially of (e.g. comprises) an amino acid sequence according to SEQ ID NO: 73. Typically, the C. acnes PITP polypeptide comprises an amino acid sequence according to SEQ ID NO: 73 or a sequence that has at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity thereto.

In some embodiments, a nucleic acid comprising a nucleotide sequence encoding a C. acnes PITP polypeptide comprises a nucleotide sequence according to any one of SEQ ID NO: 115, SEQ ID NO: 117-121, SEQ ID NO: 182-188 (e.g., SEQ ID NO: 182) or a sequence that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity thereto. In some embodiments, the nucleic acid comprises a nucleotide sequence that is least 75% identical to any one of SEQ ID NO: 115, SEQ ID NO: 117-121, SEQ ID NO: 182-188 (e.g., SEQ ID NO: 182 or SEQ ID NO: 115, such as SEQ ID NO: 182).

In one embodiment, the nucleic acid of the invention is a mRNA comprising or consisting of (e.g. consisting of) the following structural elements:
(i) a 5′ cap with the following structure:

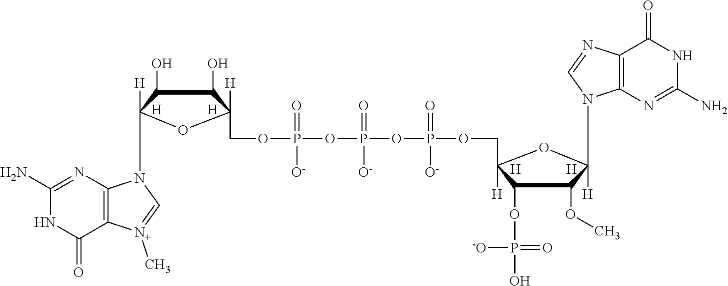

(ii) a 5′ untranslated region (5′ UTR) having the nucleic acid sequence according to SEQ ID NO: 265;
(iii) a protein coding region having the nucleic acid sequence according to SEQ ID NO: 115;
(iv) a 3′ untranslated region (3′ UTR) having the nucleic acid sequence according to SEQ ID NO: 266; and
(v) a polyA tail, optionally wherein the polyA tail comprises at least 75 adenosine nucleotides (such as about 80 adenosine nucleotides) or at least 100 adenosine nucleotides (such as about 115 adenosine nucleotides), e.g., wherein the polyA tail comprises at least 100 adenosine nucleotides.

In some embodiments, the mRNA is chemically modified and the chemical modification comprises N1-methylpseudouridine in place of every uridine. The mRNA may be encapsulated in a LNP.

### Chimeric DsA1/DsA2

Chimeric C. acnes DsA1/DsA2 polypeptides have been described previously (WO2021/165543).

C. acnes DsA1 and DsA2 proteins are homologues (paralogues) with a typical sequence identity between 60-71%, depending on which region of the protein is aligned. Excluding PT-region length polymorphism, a high degree of typically >90% sequence identity can be seen within intact DsA1 and DsA2 proteins, respectively. C. acnes DsA1 and C. acnes DsA2 are differentially expressed on the strains within the phylotypes IA1, IC and II of C. acnes. Some strains express more DsA1 polypeptide than DsA2 polypeptide and other strains express more DsA2 polypeptide than DsA1 polypeptide. Providing both a C. acnes DsA1 polypeptide and a C. acnes DsA2 polypeptide may be advantageous as this allows to target a wider range of C. acnes strains. It is possible that a DsA1 polypeptide and a DsA2 polypeptide can take over each other's function if they are targeted individually. Providing both DsA1 and DsA2 polypeptides may thus decrease the chance of immune defence evasion. Providing both DsA1 and DsA2 polypeptides may elicit an immune (e.g. antibody) response against a greater number of C. acnes strains (e.g. may elicit an antibody response against C. acnes strains expressing lower levels of one of the two polypeptides) e.g. compared to use of either DsA1 or DsA2 polypeptide alone. Providing both DsA1 and DsA2 polypeptides may thus increase immunogenicity. Provision of a chimeric C. acnes DsA1/DsA2 polypeptide as a single molecule, rather than provision of a DsA1 polypeptide and a DsA2 polypeptide as individual polypeptides, may facilitate manufacture of polypeptide and/or nucleic acids for use in the present invention.

Chimeric C. acnes DsA1/DsA2 polypeptides of the invention may be used to elicit an immune response against a C. acnes infection. Antibodies induced by chimeric C. acnes DsA1/DsA2 polypeptides were shown to bind to the surface of C. acnes bacteria. Antibodies induced by chimeric C. acnes DsA1/DsA2 polypeptides were shown to elicit opsonophagocytic killing in vitro. These antibodies may have superior cross-reactivity relative to antibodies elicited by individual (non-chimeric) C. acnes DsA1 polypeptides or C. acnes DsA2 polypeptides. Chimeric C. acnes DsA1/DsA2 polypeptides may elicit antibodies which specifically bind to both full length C. acnes DsA1 polypeptide and C. acnes DsA2 polypeptide, e.g. as determined in an ELISA assay. The antibodies elicited by DsA1 or DsA2 polypeptides may be cross-reactive against various C. acnes phylotypes. Furthermore, the antibodies elicited by chimeric DsA1/DsA2 polypeptides may be cross-reactive against various C. acnes phylotypes. The inventors have demonstrated that chimeric C. acnes DsA1/DsA2 polypeptides elicited an antibody (e.g. IgG) response when delivered as a mRNA encoding the antigen as well as in the form of recombinant protein.

Accordingly, in one aspect, the invention provides a nucleic acid that comprises a nucleotide sequence encoding a chimeric C. acnes DsA1/DsA2 polypeptide, wherein the chimeric C. acnes DsA1/DsA2 polypeptide comprises: (i) (a) a CSD2 of a C. acnes DsA1 polypeptide and (b) a CSD1 and/or a CSD3 of a C. acnes DsA2 polypeptide; or (ii) (a) a CSD2 of a C. acnes DsA2 polypeptide and (b) a CSD1 and/or a CSD3 of a C. acnes DsA1 polypeptide. The nucleic acid may be a messenger RNA (mRNA). In a further aspect, the invention provides a chimeric C. acnes DsA1/DsA2 polypeptide having an amino acid sequence comprising: (i) (a) a CSD2 of a C. acnes DsA1 polypeptide and (b) a CSD1 and/or a CSD3 of a C. acnes DsA2 polypeptide; or (ii) (a) a CSD2 of a C. acnes DsA2 polypeptide and (b) a CSD1 and/or a CSD3 of a C. acnes DsA1 polypeptide. A chimeric C. acnes DsA1/DsA2 polypeptide for use in the present invention, e.g. delivered as a mRNA or as a recombinant protein, may elicit an immune response (e.g. antibody response) in a subject. Such antibodies may be cross-reactive antibodies which specifically bind to both a native C. acnes DsA1 polypeptide and a native C. acnes DsA2 polypeptide. Such antibody response may be cross-reactive against various C. acnes phylotypes (e.g. phylotypes IA1, IA2, IB IC, II and III).

In some embodiments, a chimeric C. acnes DsA1/DsA2 polypeptide comprises:
(i) a NSR of a C. acnes DsA1 polypeptide or a NSR of a C. acnes DsA2 polypeptide (e.g. of a C. acnes DsA1 polypeptide),
(ii) a CSD1 of a C. acnes DsA1 polypeptide or a CSD1 of a C. acnes DsA2 polypeptide (e.g. of a C. acnes DsA1 polypeptide),
(iii) optionally a SRI of a C. acnes DsA1 polypeptide (or a portion thereof) and/or a SRI of a C. acnes DsA2 polypeptide (or a portion thereof),
(iv) a CSD2 of a C. acnes DsA1 polypeptide or a CSD2 of a C. acnes DsA2 polypeptide (e.g. of a C. acnes DsA2 polypeptide),
(v) optionally a SR2 of a C. acnes DsA1 polypeptide (or a portion thereof) and/or a SR2 of a C. acnes DsA2 polypeptide (or a portion thereof), and
(vi) a CSD3 of a C. acnes DsA1 polypeptide or a CSD3 of a C. acnes DsA2 polypeptide (e.g. of a C. acnes DsA1 polypeptide).

As described in WO2021/165543, the SRs within C. acnes DsA1 polypeptides and C. acnes DsA2 polypeptides are typically regions which are enriched in intrinsically disordered polypeptide sequences (i.e., they are largely unstructured). The SRs share the property of linking (“spacing”) two structural domains, such as the CSDs (such as CSD1, CSD2 and CSD3). Therefore, the CSDs from a C. acnes DsA1 polypeptide and a C. acnes DsA2 polypeptide may be combined by appropriately engineering the SRs such that their length (number of amino acid residues) is conserved. Advantageously, this design strategy may retain the structural integrity of the individual structured domains (e.g., the CSDs) in the resulting chimeric DsA1/DsA2 polypeptides.

In some embodiments, a chimeric C. acnes DsA1/DsA2 polypeptide of the invention comprises (e.g. from N terminus to C terminus) amino acid residues 21-102 of SEQ ID NO: 204, the amino acid residues 121-240 of SEQ ID NO: 205 and the amino acid residues 250-295 of SEQ ID NO: 204.

In some embodiments, the chimeric C. acnes DsA1/DsA2 polypeptide comprises a sequence according to any one of SEQ ID NO: 28-30, SEQ ID NO: 39 or SEQ ID NO: 70-72 or SEQ ID NO: 81 or a sequence having at least 90%, at least 91%, at least 92%, at least 93%, at least 94%, at least 95%, at least 96%, at least 97%, at least 98%, or at least 99% identity thereto. In some embodiments, the chimeric C. acnes DsA1/DsA2 polypeptide comprises a sequence having at least 95% identity to any one of SEQ ID NO: 28-30, SEQ ID NO: 39 or SEQ ID NO: 70-72 or SEQ ID NO: 81. Preferably, the chimeric C. acnes DsA1/DsA2 polypeptide comprises an amino acid sequence according to SEQ ID NO: 70 or a sequence that has at least 90%, at least 91%, at least 92%, at least 93%, at least 94%, at least 95%, at least 96%, at least 97%, at least 98%, or at least 99% identity thereto.

In some embodiments, the chimeric C. acnes DsA1/DsA2 polypeptide comprises, consists of or consists essentially of (e.g. comprises) an amino acid sequence according to SEQ ID NO: 70.

In some embodiments, a nucleic acid comprising a nucleotide sequence encoding the chimeric C. acnes DsA1/DsA2 polypeptide comprises a nucleotide sequence according to SEQ ID NO: 113-114, SEQ ID NO: 179, SEQ ID NO: 123 or SEQ ID NO: 190 (e.g., SEQ ID NO: 179) or a sequence that has at least 75%, at least 80%, at least 85%, at least 90%, at least 91%, at least 92%, at least 93%, at least 94%, at least 95%, at least 96%, at least 97%, at least 98%, or at least 99% identity thereto. In some embodiments, a nucleic acid comprising a nucleotide sequence encoding the chimeric C. acnes DsA1/DsA2 polypeptide comprises a nucleotide sequence that is least 85% identical to SEQ ID NO: 113-114, SEQ ID NO: 179, SEQ ID NO: 123 or SEQ ID NO: 190 (e.g., SEQ ID NO: 179). In some embodiments, a nucleic acid comprising a nucleotide sequence encoding the chimeric C. acnes DsA1/DsA2 polypeptide comprises or consists of a nucleotide sequence encoding a secretion signal peptide sequence (e.g., a viral secretion signal peptide sequence described herein) and a nucleotide sequence according to SEQ ID NO: 179. In some embodiments, a nucleic acid comprising a nucleotide sequence encoding the chimeric C. acnes DsA1/DsA2 polypeptide comprises or consists of a nucleotide sequence according to SEQ ID NO: 113.

In one embodiment, the nucleic acid of the invention is a mRNA comprising or consisting of (e.g. consisting of) the following structural elements:
(i) a 5′ cap with the following structure:

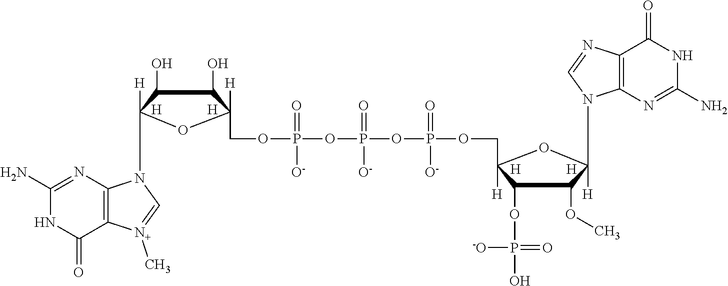

(ii) a 5′ untranslated region (5′ UTR) having the nucleic acid sequence according to SEQ ID NO: 265;
(iii) a protein coding region having the nucleic acid sequence according to SEQ ID NO: 113;
(iv) a 3′ untranslated region (3′ UTR) having the nucleic acid sequence according to SEQ ID NO: 266; and
(v) a polyA tail, optionally wherein the polyA tail comprises at least 75 adenosine nucleotides (such as about 80 adenosine nucleotides) or at least 100 adenosine nucleotides (such as about 115 adenosine nucleotides), e.g., wherein the polyA tail comprises at least 100 adenosine nucleotides.

In some embodiments, the mRNA is chemically modified and the chemical modification comprises N1-methylpseudouridine in place of every uridine. The mRNA may be encapsulated in a LNP.

### Chimeric DsA1/DsA2/PITP

Chimeric C. acnes DsA1/DsA2/PITP polypeptides have been described previously (WO2021/165543).

DsA1 polypeptides and DsA2 polypeptides may be expressed on C. acnes phylotypes IA1, IA2, IC and II. Antibodies induced by DsA1 polypeptides and DsA2 polypeptides may be unable to bind to C. acnes MLST phylotypes IB and III. Antibodies against C. acnes PITP polypeptides on the other hand may be able to bind to C. acnes type IB and C. acnes Type III. Therefore, PITP may be an antigen that can complement the immune response induced by other C. acnes antigens, e.g. DsA1 and/or DsA2. The combination of a C. acnes PITP polypeptide and a chimeric C. acnes DsA1/DsA2 polypeptide (delivered as a mRNA or in the form of a recombinant protein) may elicit an immune response against a greater number of C. acnes strains or phylotypes compared to use of individual antigens. A combination of DsA1 and/or DsA2 with PITP, delivered as a mRNA or in the form of a recombinant protein may provide an immune response (e.g, an antibody response) that is cross-reactive against a greater number of strains or phylotypes of C. acnes relative to using only DsA1 and/or DsA2. A C. acnes PITP polypeptide may be provided as a separate polypeptide molecule (delivered as a mRNA or in the form of a recombinant protein) or as part of a chimeric C. acnes DsA1/DsA2/PITP polypeptide (delivered as a mRNA or in the form of a recombinant protein).

Chimeric C. acnes DsA1/DsA2/PITP polypeptides of the invention (delivered as a mRNA or in the form of a recombinant protein) may be used to elicit an immune response against a C. acnes infection. A chimeric C. acnes DsA1/DsA2/PITP polypeptide (delivered as a mRNA or in the form of a recombinant protein) may elicit an immune response against a greater number of C. acnes strains compared to use of individual antigens (e.g. DsA1, DsA2 or PITP), e.g. a cross-reactive immune response. Provision of a chimeric C. acnes DsA1/DsA2/PITP polypeptide, rather than provision of a C. acnes DsA1 polypeptide, a C. acnes DsA2 polypeptide and a C. acnes PITP polypeptide as individual polypeptides (or a chimeric C. acnes DsA1/DsA2 polypeptide and a C. acnes PITP polypeptide), may facilitate manufacture of polypeptide and/or nucleic acids for use in the present invention.

Chimeric C. acnes DsA1/DsA2/PITP polypeptides may elicit an antibody response which is cross-reactive against various C. acnes phylotypes. The inventors have demonstrated that chimeric C. acnes DsA1/DsA2/PITP polypeptides elicited an antibody (e.g. IgG) response when delivered as a mRNA encoding the antigen as well as in the form of recombinant protein. Antibodies induced by chimeric C. acnes DsA1/DsA2/PITP polypeptides were shown to bind to the surface of C. acnes bacteria.

Accordingly, in one aspect, the invention provides a nucleic acid that comprises a nucleotide sequence encoding a chimeric C. acnes DsA1/DsA2/PITP polypeptide, wherein the chimeric C. acnes DsA1/DsA2/PITP polypeptide comprises a ENFD of a C. acnes PITP polypeptide and:
(i) (a) a CSD2 of a C. acnes DsA1 polypeptide and (b) a CSD1 and/or a CSD3 of a C. acnes DsA2 polypeptide; or
(ii) (a) a CSD2 of a C. acnes DsA2 polypeptide and (b) a CSD1 and/or a CSD3 of a C. acnes DsA1 polypeptide.

The nucleic acid may be a messenger RNA (mRNA).

In a further aspect, the invention provides a chimeric C. acnes DsA1/DsA2/PITP polypeptide comprising a ENFD of a C. acnes PITP polypeptide and:
(i) (a) a CSD2 of a C. acnes DsA1 polypeptide and (b) a CSD1 and/or a CSD3 of a C. acnes DsA2 polypeptide; or
(ii) (a) a CSD2 of a C. acnes DsA2 polypeptide and (b) a CSD1 and/or a CSD3 of a C. acnes DsA1 polypeptide.

A C. acnes DsA1/DsA2/PITP polypeptide for use in the present invention, e.g. delivered as a mRNA or as a recombinant protein, may elicit an immune response (e.g. antibodies) in a subject. Such antibodies may include antibodies which specifically bind to a native C. acnes DsA1 polypeptide, antibodies which specifically bind to a native C. acnes DsA2 polypeptide and/or antibodies which specifically bind to a native C. acnes PITP polypeptide. Such antibodies may be cross-reactive against various C. acnes phylotypes (e.g. phylotypes IA1, IA2, IB IC, II and III).).

In some embodiments, a chimeric C. acnes DsA1/DsA2 polypeptide comprises a CSD2 of a C. acnes DsA2 polypeptide, a CSD1 of a C. acnes DsA1 polypeptide, a CSD3 of a C. acnes DsA1 polypeptide and a ENFD of a C. acnes PITP polypeptide. In some embodiments, a chimeric C. acnes DsA1/DsA2/PITP polypeptide comprises:
(a) (i) a NSR of a C. acnes DsA1 polypeptide or a NSR of a C. acnes DsA2 polypeptide (e.g. of a C. acnes DsA1 polypeptide),
   (ii) a CSD1 of a C. acnes DsA1 polypeptide or a CSD1 of a C. acnes DsA2 polypeptide (e.g. of a C. acnes DsA1 polypeptide),
   (iii) optionally a SRI of a C. acnes DsA1 polypeptide (or a portion thereof) and/or a SRI of a C. acnes DsA2 polypeptide (or a portion thereof),
   (iv) a CSD2 of a C. acnes DsA1 polypeptide or a CSD2 of a C. acnes DsA2 polypeptide (e.g. of a C. acnes DsA2 polypeptide),
   (v) optionally a SR2 of a C. acnes DsA1 polypeptide (or a portion thereof) and/or a SR2 of a C. acnes DsA2 polypeptide (or a portion thereof), and
   (vi) a CSD3 of a C. acnes DsA1 polypeptide or a CSD3 of a C. acnes DsA2 polypeptide (e.g. of a C. acnes DsA1 polypeptide); and
(b) a ENFD of a C. acnes PITP polypeptide.

As described in WO2021/165543, the SRs within C. acnes DsA1 polypeptides and C. acnes DsA2 polypeptides and PITP are typically regions which are enriched in intrinsically disordered polypeptide sequences (i.e., they are largely unstructured). The SRs share the property of linking (“spacing”) two structural domains, such as the CSDs (such as CSD1, CSD2 and CSD3 of C. acnes DsA1 polypeptides and C. acnes DsA2 polypeptides), the ENFD of C. acnes PITP polypeptides or the HbD of C. acnes PITP polypeptides. Therefore, CSDs from a C. acnes DsA1 polypeptide and a C. acnes DsA2 polypeptide, ENFD of a C. acnes PITP polypeptide and/or the HbD of C. acnes PITP polypeptide may be combined by interspersing them with SRs of a similar length (number of amino acid residues) to that of the SRs in a native C. acnes polypeptide. Advantageously, this design strategy may retain the structural integrity of the individual structured domains (e.g., the CSDs, ENFD and HbD) in the resulting chimeric DsA1/DsA2/PITP polypeptides.

In some embodiments, the chimeric C. acnes DsA1/DsA2/PITP polypeptide comprises an amino acid sequence according to any one of SEQ ID NO: 38, SEQ ID NO: 40-41, SEQ ID NO: 80, SEQ ID NO: 82-83 or SEQ ID NO: 367-368 (e.g., SEQ ID NO: 80, 82, 83 or 368) or a sequence that has at least 90%, at least 91%, at least 92%, at least 93%, at least 94%, at least 95%, at least 96%, at least 97%, at least 98%, or at least 99% identity thereto. In some embodiments, the chimeric C. acnes DsA1/DsA2/PITP polypeptide comprises a sequence having at least 90% identity to any one of SEQ ID NO: 38, SEQ ID NO: 40-41, SEQ ID NO: 80, SEQ ID NO: 82-83 or SEQ ID NO: 367-368 (e.g., SEQ ID NO: 80, 82, 83 or 368). In some embodiments, the chimeric C. acnes DsA1/DsA2/PITP polypeptide comprises a sequence having at least 95% identity to any one of SEQ ID NO: 38, SEQ ID NO: 40-41, SEQ ID NO: 80, SEQ ID NO: 82-83 or SEQ ID NO: 367-368 (e.g., SEQ ID NO: 80, 82, 83 or 368).

In some embodiments, the chimeric C. acnes DsA1/DsA2/PITP polypeptide consists of an amino acid sequence according to SEQ ID NO: 80, 82, 83 or 368.

In some embodiments, a nucleic acid comprising a nucleotide sequence encoding the chimeric C. acnes DsA1/DsA2/PITP polypeptide comprises a nucleotide sequence according to any one of SEQ ID NO: 122, SEQ ID NO 124-125, SEQ ID NO: 189 or SEQ ID NO: 191-192 (e.g., SEQ ID NO: 189 or 192) or a sequence that has at least 75%, at least 80%, at least 85%, at least 90%, at least 91%, at least 92%, at least 93%, at least 94%, at least 95%, at least 96%, at least 97%, at least 98%, or at least 99% identity thereto. In some embodiments, a nucleic acid comprising a nucleotide sequence encoding the chimeric C. acnes DsA1/DsA2/PITP polypeptide comprises a nucleotide sequence that is least 85% identical to any one of SEQ ID NO: 122, SEQ ID NO 124-125, SEQ ID NO: 189 or SEQ ID NO: 191-192 (e.g., SEQ ID NO: 189 or 192).

In some embodiments, a nucleic acid comprising a nucleotide sequence encoding the chimeric C. acnes DsA1/DsA2 polypeptide consists of a nucleotide sequence encoding a secretion signal peptide sequence (e.g., a viral secretion signal peptide sequence described herein) and a sequence according to any one of SEQ ID NO: 189 or SEQ ID NO: 191-192.

### Chimeric DsA1/DsA2/PITP/CAMP2

The inventors have designed chimeric C. acnes DsA1/DsA2/PITP/CAMP2 polypeptides (also referred to herein as chimeric C. acnes CAMP2/DsA1/DsA2/PITP polypeptides). Chimeric C. acnes DsA1/DsA2/PITP/CAMP2 polypeptides of the present invention may elicit an immune (e.g. antibody) response. This response may be a protective immune (e.g. antibody) response. Chimeric C. acnes DsA1/DsA2/PITP/CAMP2 polypeptides may elicit an immune response (e.g. antibodies) against native C. acnes DsA1 polypeptide, an immune response (e.g. antibodies) against native C. acnes DsA2 polypeptide, an immune response (e.g. antibodies) against native C. acnes PITP polypeptide and/or an immune response (e.g. antibodies) against native C. acnes CAMP2 polypeptide. Such antibodies may be cross-reactive against various C. acnes MLST phylotypes (e.g. phylotypes IA1, IA2, IB IC, II and III). The antibodies elicited by chimeric C. acnes DsA1/DsA2/PITP/CAMP2 polypeptides may neutralise biological activity of CAMP2 polypeptide, such as CAMP2 inflammatory activity. The antibodies elicited by the chimeric C. acnes DsA1/DsA2/PITP/CAMP2 polypeptides may opsonize C. acnes bacteria. Opsonization may target immune effector cells such as phagocytes to C. acnes bacteria. This may lead to killing of C. acnes bacteria by such immune effector cells (e.g. phagocytic killing). Chimeric C. acnes DsA1/DsA2/PITP/CAMP2 polypeptides may elicit antibodies that can neutralise biological activity of CAMP2 polypeptide, such as CAMP2 inflammatory activity. Chimeric C. acnes DsA1/DsA2/PITP/CAMP2 polypeptides may therefore elicit (a) antibodies that neutralise biological activity of a C. acnes CAMP2 polypeptide, such as inflammatory activity and (b) antibodies that opsonize C. acnes bacteria and recruit immune effector cells such as phagocytes. Thus, immunisation with chimeric C. acnes DsA1/DsA2/PITP/CAMP2 polypeptides of the invention (e.g. delivered as a mRNA or as a recombinant protein) may provide a more effective immune response (e.g. more effective killing of C. acnes bacteria in a subject) than immunisation with chimeric DsA1/DsA2/PITP polypeptides or individual DsA1, DsA2 or PITP antigens or a CAMP2 polypeptide on its own.

The inventors have demonstrated that chimeric C. acnes DsA1/DsA2/PITP/CAMP2 polypeptides elicited an antibody (e.g. IgG) response against DsA1, DsA2, PITP and CAMP2 antigens when delivered as a mRNA encoding the polypeptide. Antibodies induced by the chimeric C. acnes DsA1/DsA2/PITP/CAMP2 polypeptides were shown to bind to the surface of C. acnes bacteria. Antibodies induced by the chimeric C. acnes DsA1/DsA2/PITP/CAMP2 polypeptides were shown to elicit opsonophagocytic killing in vitro. The inventors have also shown that antibodies induced by the chimeric C. acnes DsA1/DsA2/PITP/CAMP2 polypeptides reduced the co-hemolytic activity of C. acnes CAMP2 polypeptide.

Combining DsA1, DsA2, PITP and CAMP2 antigens in a single chimeric polypeptide may advantageously reduce reactogenicity of a vaccine, e.g. in the context of the chimeric polypeptide being delivered in the form of a single mRNA, e.g. because a vaccine formulation of a single mRNA may use less cationic lipid or LNP than a vaccine formulation comprising several different mRNAs. Combining DsA1, DsA2, PITP and CAMP2 antigens in a single chimeric polypeptide may also facilitate the manufacturing of a vaccine as it is easier, quicker and less costly to make a single nucleic acid or polypeptide combining the four antigens rather than separate nucleic acids or polypeptides for the different antigens.

As described above herein, the design strategy may exploit the structural integrity of the individual structured domains (e.g., the CSDs of DsA1 and DsA2 and ENFD of PITP) in the resulting chimeric DsA1/DsA2/PITP/CAMP2 polypeptides. The design strategy may also exploit flexible linker domain of C. acnes CAMP2 polypeptide and/or a flexible linker-like sequence at the C terminus of the chimeric C. acnes DsA1/DsA2/PITP polypeptide.

Accordingly, in one aspect, the invention provides a nucleic acid that comprises a nucleotide sequence encoding a chimeric C. acnes DsA1/DsA2/PITP/CAMP2 polypeptide, wherein the chimeric C. acnes DsA1/DsA2/PITP/CAMP2 polypeptide comprises a C. acnes DsA1 or an immunogenic fragment thereof, a C. acnes DsA2 or an immunogenic fragment thereof, a C. acnes PITP polypeptide or an immunogenic fragment thereof and a C. acnes CAMP2 polypeptide or an immunogenic fragment thereof. Typically, the nucleic acid is a messenger RNA (mRNA).

In a further aspect, the invention provides a chimeric C. acnes DsA1/DsA2/PITP/CAMP2 polypeptide comprising a C. acnes DsA1 or an immunogenic fragment thereof, a C. acnes DsA2 or an immunogenic fragment thereof, a C. acnes PITP polypeptide or an immunogenic fragment thereof and a C. acnes CAMP2 polypeptide or an immunogenic fragment thereof.

In some embodiments, the immunogenic fragment of C. acnes DsA1 polypeptide is a CSD2 of a C. acnes DsA1 polypeptide. In some embodiments, the immunogenic fragment of C. acnes DsA2 polypeptide is a CSD2 of a C. acnes DsA2 polypeptide.

In some embodiments, a second conserved sub-domain (“CSD2”) of C. acnes DsA1 polypeptide corresponds to amino acid residues 120-239 of SEQ ID NO: 204. See the section “DsA1” above for description of other domains of a C. acnes DsA1 polypeptide and their boundaries. As is known in the art, the positions of the CSD2 and other domains within other native C. acnes DsA1 polypeptides may be determined by aligning the polypeptide sequence of a given DsA1 polypeptide with that of SEQ ID NO: 204, and identifying the regions of the DsA1 polypeptide which have high sequence identity, respectively, to that of the CSD2 and other domains of SEQ ID NO: 204, respectively.

In some embodiments, a second conserved sub-domain (“CSD2”) of C. acnes DsA2 polypeptide corresponds to amino acid residues 121-240 of SEQ ID NO: 205. See the section “DsA2” above for description of other domains of a C. acnes DsA2 polypeptide and their boundaries. As is known in the art, the positions of the CSD2 and other domains within other native C. acnes DsA2 polypeptides may be determined by aligning the polypeptide sequence of a given DsA2 polypeptide with that of SEQ ID NO: 205, and identifying the regions of the DsA2 polypeptide which have high sequence identity, respectively, to that of the CSD2 and other domains of SEQ ID NO: 205, respectively.

In some embodiments, the immunogenic fragment of a C. acnes PITP polypeptide comprises a ENFD of a C. acnes PITP polypeptide.

In some embodiments, an extended neocarzinostatin family domain (“ENFD”) of C. acnes PITP corresponds to amino acid residues 1-133 of SEQ ID NO: 206. See the section “PITP” above for description of other domains of a C. acnes PITP polypeptide and their boundaries. As is known in the art, the positions of the ENFD and other domains within other native C. acnes PITP polypeptides may be determined by aligning the polypeptide sequence of a given PITP polypeptide with that of SEQ ID NO: 206, and identifying the regions of the PITP polypeptide which have high sequence identity, respectively, to that of the ENFD and other domains of SEQ ID NO: 206, respectively.

In some embodiments, the immunogenic fragment of C. acnes CAMP2 polypeptide comprises a N-terminal domain of C. acnes CAMP2 polypeptide. In some embodiments, the immunogenic fragment of C. acnes CAMP2 polypeptide comprises a N-terminal domain of C. acnes CAMP2 polypeptide and a linker domain of C. acnes CAMP2 polypeptide. In some embodiments, the immunogenic fragment of C. acnes CAMP2 polypeptide comprises a C-terminal domain of C. acnes CAMP2 polypeptide. In some embodiments, the immunogenic fragment of C. acnes CAMP2 polypeptide comprises a linker domain of C. acnes CAMP2 polypeptide and a C-terminal domain of C. acnes CAMP2 polypeptide.

In some embodiments, a N-terminal domain of a C. acnes CAMP2 polypeptide corresponds to residues 29-176 of SEQ ID NO: 202. In some embodiments, a C-terminal domain of a C. acnes CAMP2 polypeptide corresponds to residues 189-267 of SEQ ID NO: 202. In some embodiments, a linker domain of a C. acnes CAMP2 polypeptide corresponds to residues 177-188 of SEQ ID NO: 202. As is known in the art, the positions of the N-terminal domain, C-terminal domain and linker domain within other C. acnes CAMP2 polypeptides may be determined by aligning the polypeptide sequence of a given CAMP2 polypeptide with that of SEQ ID NO: 202, and identifying the regions of the CAMP2 polypeptide which have high sequence identity to that the sequence of the N-terminal domain, C-terminal domain and linker domain of SEQ ID NO: 202, respectively. Analysis of 430 CAMP2 polypeptide sequences from naturally occurring strains of C. acnes showed that there was a high degree of sequence conservation between the CAMP2 sequences (see “CAMP2” section above). FIGS. 44A-44C show an exemplary alignment of CAMP2 polypeptide sequences from naturally occurring C. acnes strains relative to SEQ NO: 202 (i.e., KPA171202_REF in FIGS. 44A-44C). Alignments such as the one in FIGS. 44A-44C can be used to identify the positions of the N-terminal domain, C-terminal domain and linker domain in sequences other than SEQ ID NO: 202.

In some embodiments, any two or more of (1) the C. acnes DsA1 polypeptide or an immunogenic fragment thereof, (2) the C. acnes DsA2 polypeptide or an immunogenic fragment thereof, (3) the C. acnes PITP polypeptide or an immunogenic fragment thereof, and (4) the C. acnes CAMP2 polypeptide or an immunogenic fragment thereof, are attached via linkers (such as the linkers described in section “Linkers” below). In other embodiments, (1) the C. acnes DsA1 polypeptide or an immunogenic fragment thereof, (2) the C. acnes DsA2 polypeptide or an immunogenic fragment thereof, (3) the C. acnes PITP polypeptide or an immunogenic fragment thereof, and (4) the C. acnes CAMP2 polypeptide or an immunogenic fragment thereof are directly fused to each other (i.e., there is no linker, such as an amino acid linker, connecting the DsA1, DsA2, PITP and CAMP2 polypeptides or their immunogenic fragments to each other). In some embodiments, such chimeric C. acnes DsA1/DsA2/PITP/CAMP2 polypeptides comprise a linker domain of C. acnes CAMP2 polypeptide.

In one embodiment, the chimeric C. acnes DsA1/DsA2/PITP/CAMP2 polypeptide comprises:
(a) a chimeric C. acnes DsA1/DsA2 polypeptide, e.g. as described herein;
(b) an immunogenic fragment of a C. acnes PITP polypeptide comprising a ENFD of a C. acnes PITP polypeptide; and
(c) a C. acnes CAMP2 polypeptide or an immunogenic fragment thereof.

In some embodiments, the chimeric C. acnes DsA1/DsA2 polypeptide comprises (i) (1) a CSD2 of a C. acnes DsA1 polypeptide and (2) a CSD1 and/or a CSD3 of a C. acnes DsA2 polypeptide; or (ii) (1) a CSD2 of a C. acnes DsA2 polypeptide and (2) a CSD1 and/or a CSD3 of a C. acnes DsA1 polypeptide. See above and the sections “DsA1” and “DsA2” for description of domains of C. acnes DsA1 and DsA2 polypeptides, respectively, and their boundaries. In some embodiments, the chimeric C. acnes DsA1/DsA2/PITP/CAMP2 polypeptide is as defined in (ii). Typically, the chimeric C. acnes DsA1/DsA2/PITP/CAMP2 polypeptide comprises a CSD1 of a C. acnes DsA1 polypeptide, a CSD2 of a C. acnes DsA2 polypeptide and a CSD3 of a C. acnes DsA1 polypeptide.

In some embodiments, the chimeric C. acnes DsA1/DsA2 polypeptide of (a) comprises a sequence according to SEQ ID NO: 70, or a sequence having at least 90%, at least 91%, at least 92%, at least 93%, at least 94%, at least 95%, at least 96%, at least 97%, at least 98%, or at least 99% identity thereto.

In some embodiments, the immunogenic fragment of a C. acnes PITP polypeptide comprising a ENFD of a C. acnes PITP polypeptide in (b) comprises the sequence corresponding to amino acid residues 1-133 of SEQ ID NO: 73 or a sequence having at least 90%, at least 91%, at least 92%, at least 93%, at least 94%, at least 95%, at least 96%, at least 97%, at least 98%, or at least 99% identity to the sequence of amino acid residues 1-133 of SEQ ID NO: 73. In some embodiments, the immunogenic fragment of a C. acnes PITP polypeptide comprising a ENFD of a C. acnes PITP polypeptide in (b) comprises the sequence corresponding to amino acid residues 1-146 of SEQ ID NO: 73 or a sequence having at least 90%, at least 91%, at least 92%, at least 93%, at least 94%, at least 95%, at least 96%, at least 97%, at least 98%, or at least 99% identity to the sequence of amino acid residues 1-146 of SEQ ID NO: 73. Typically, the immunogenic fragment of a C. acnes PITP polypeptide comprising a ENFD of a C. acnes PITP polypeptide in (b) comprises the sequence corresponding to amino acid residues 1-146 of SEQ ID NO: 73.

In some embodiments, (a) and (b) comprise SEQ ID NO: 80 or a sequence having at least 90%, at least 91%, at least 92%, at least 93%, at least 94%, at least 95%, at least 96%, at least 97%, at least 98%, or at least 99% identity thereto. In some embodiments, (a) and (b) comprise SEQ ID NO: 82 or a sequence having at least 90%, at least 91%, at least 92%, at least 93%, at least 94%, at least 95%, at least 96%, at least 97%, at least 98%, or at least 99% identity thereto. In some embodiments, (a) and (b) comprise SEQ ID NO: 83 or a sequence having at least 90%, at least 91%, at least 92%, at least 93%, at least 94%, at least 95%, at least 96%, at least 97%, at least 98%, or at least 99% identity thereto. Typically, (a) and (b) comprise SEQ ID NO: 80.

In some embodiments, (c) is a C. acnes CAMP2 polypeptide, e.g., a full-length C. acnes CAMP2 polypeptide, typically a mature form of full-length CAMP2 polypeptide of C. acnes (without the native signal peptide sequence). In some embodiments, (c) comprises SEQ ID NO: 203 or a sequence having at least 90%, at least 91%, at least 92%, at least 93%, at least 94%, at least 95%, at least 96%, at least 97%, at least 98%, or at least 99% identity thereto. Typically, (c) comprises SEQ ID NO: 203.

In some embodiments, the immunogenic fragment of a C. acnes CAMP2 polypeptide in (c) comprises a N-terminal domain of a C. acnes CAMP2 polypeptide. In some embodiments, (c) comprises amino acid residues 29-176 of SEQ ID NO: 202 or a sequence having at least 90%, at least 91%, at least 92%, at least 93%, at least 94%, at least 95%, at least 96%, at least 97%, at least 98%, or at least 99% identity to the sequence of amino acid residues 29-176 of SEQ ID NO: 202.

In some embodiments, the immunogenic fragment of a C. acnes CAMP2 polypeptide in (c) comprises a N-terminal domain of a C. acnes CAMP2 polypeptide and a linker domain of a C. acnes CAMP2 polypeptide. In some embodiments, (c) comprises amino acid residues 29-188 of SEQ ID NO: 202 or a sequence having at least 90%, at least 91%, at least 92%, at least 93%, at least 94%, at least 95%, at least 96%, at least 97%, at least 98%, or at least 99% identity to the sequence of amino acid residues 29-188 of SEQ ID NO: 202.

In some embodiments, the immunogenic fragment of a C. acnes CAMP2 polypeptide in (c) comprises a C-terminal domain of a C. acnes CAMP2 polypeptide. In some embodiments, (c) comprises amino acid residues 189-267 of SEQ ID NO: 202 or a sequence having at least 90%, at least 91%, at least 92%, at least 93%, at least 94%, at least 95%, at least 96%, at least 97%, at least 98%, or at least 99% identity to the sequence of amino acid residues 189-267 of SEQ ID NO: 202.

In some embodiments, the immunogenic fragment of a C. acnes CAMP2 polypeptide in (c) comprises a linker domain of a C. acnes CAMP2 polypeptide and a C-terminal domain of a C. acnes CAMP2 polypeptide. In some embodiments, (c) comprises amino acid residues 177-267 of SEQ ID NO: 202 or a sequence having at least 90%, at least 91%, at least 92%, at least 93%, at least 94%, at least 95%, at least 96%, at least 97%, at least 98%, or at least 99% identity to the sequence of amino acid residues 177-267 of SEQ ID NO: 202.

In some embodiments, any two or more of (a), (b) and (c) are attached via linkers (such as the linkers described in section “Linkers” below). Typically, (a), (b) and (c) are directly fused to each other (i.e., there is no linker, such as an amino acid linker, connecting (a), (b) and (c)), e.g. in the order specified below. In some embodiments, (c) comprises a linker domain of C. acnes CAMP2 polypeptide.

In some embodiments, the chimeric C. acnes DsA1/DsA2/PITP/CAMP2 polypeptide comprises, from N-terminus to C-terminus: (a), (b) and (c). In other embodiments, the chimeric C. acnes DsA1/DsA2/PITP/CAMP2 polypeptide comprises, from N-terminus to C-terminus: (c), (a) and (b). Typically, the chimeric C. acnes DsA1/DsA2/PITP/CAMP2 polypeptide comprises, from N-terminus to C-terminus: (c), (a) and (b).

In some embodiments, the chimeric C. acnes DsA1/DsA2/PITP/CAMP2 polypeptide comprises a transmembrane domain (TMB) sequence, e.g. a TMB as defined herein (see the section “Heterologous transmembrane domains (TMBs)” below). In certain embodiments, the TMB sequence is positioned at the N-terminus of the chimeric C. acnes DsA1/DsA2/PITP/CAMP2 polypeptide. In other embodiments, the TMB sequence is positioned at the C-terminus of the chimeric C. acnes DsA1/DsA2/PITP/CAMP2 polypeptide. Typically, the TMB sequence is positioned at the C-terminus of the chimeric C. acnes DsA1/DsA2/PITP/CAMP2 polypeptide.

In some embodiments, the chimeric C. acnes DsA1/DsA2/PITP/CAMP2 polypeptide comprises a sequence according to any one of SEQ ID NO: 373-376 (e.g. SEQ ID NO: 373 or 374) or a sequence having at least 90%, at least 91%, at least 92%, at least 93%, at least 94%, at least 95%, at least 96%, at least 97%, at least 98%, or at least 99% identity thereto. In some embodiments, the chimeric C. acnes DsA1/DsA2/PITP/CAMP2 polypeptide comprises a sequence according to SEQ ID NO: 373 or a sequence having at least 90%, at least 91%, at least 92%, at least 93%, at least 94%, at least 95%, at least 96%, at least 97%, at least 98%, or at least 99% identity thereto, and a TMB sequence (e.g. a sequence according to SEQ ID NO: 84). Typically, the chimeric C. acnes DsA1/DsA2/PITP/CAMP2 polypeptide comprises (1) a sequence according to SEQ ID NO: 374 or a sequence having at least 90%, at least 91%, at least 92%, at least 93%, at least 94%, at least 95%, at least 96%, at least 97%, at least 98%, or at least 99% identity thereto; or (2) a sequence according to SEQ ID NO: 373, or a sequence having at least 90%, at least 91%, at least 92%, at least 93%, at least 94%, at least 95%, at least 96%, at least 97%, at least 98%, or at least 99% identity thereto, and a TMB sequence.

In some embodiments, the nucleic acid comprising a nucleotide sequence encoding the chimeric C. acnes DsA1/DsA2/PITP/CAMP2 polypeptide comprises a nucleotide sequence encoding a secretion signal peptide sequence as described herein (e.g. a secretion signal peptide sequence as described in the section “Secretion signal peptide (SS) sequences” below).

In some embodiments, the nucleic acid comprising a nucleotide sequence encoding the chimeric C. acnes DsA1/DsA2/PITP/CAMP2 polypeptide comprises a sequence according to any one of SEQ ID NO: 386-390 or SEQ ID NO: 393 (e.g. any one of SEQ ID NO: 387 or a sequence having at least 75%, at least 80%, at least 85%, at least 90%, at least 91%, at least 92%, at least 93%, at least 94%, at least 95%, at least 96%, at least 97%, at least 98%, or at least 99% identity thereto. In some embodiments, the nucleic acid comprising a nucleotide sequence encoding the chimeric C. acnes DsA1/DsA2/PITP/CAMP2 polypeptide comprises a sequence according to SEQ ID NO: 384 or SEQ ID NO: 385 or a sequence having at least 75%, at least 80%, at least 85%, at least 90%, at least 91%, at least 92%, at least 93%, at least 94%, at least 95%, at least 96%, at least 97%, at least 98%, or at least 99% identity thereto, and a nucleotide sequence encoding a TMB sequence (e.g. a nucleotide sequence encoding SEQ ID NO: 84). In some embodiments, the nucleotide sequence encoding SEQ ID NO: 84 is as defined in SEQ ID NO: 395 or 396. Typically, the nucleic acid comprising a nucleotide sequence encoding the chimeric C. acnes DsA1/DsA2/PITP/CAMP2 polypeptide comprises (1) a sequence according to SEQ ID NO: 387, or a sequence having at least 75%, at least 80%, at least 85%, at least 90%, at least 91%, at least 92%, at least 93%, at least 94%, at least 95%, at least 96%, at least 97%, at least 98%, or at least 99% identity thereto; or (2) a sequence according to SEQ ID NO: 384 or SEQ ID NO: 385, or a sequence having at least 75%, at least 80%, at least 85%, at least 90%, at least 91%, at least 92%, at least 93%, at least 94%, at least 95%, at least 96%, at least 97%, at least 98%, or at least 99% identity thereto, and a nucleotide sequence encoding a TMB sequence.

In some embodiments, the nucleic acid comprising a nucleotide sequence encoding the chimeric C. acnes DsA1/DsA2/PITP/CAMP2 polypeptide comprises a nucleotide sequence according to any one of SEQ ID NO: 377-383 or SEQ ID NO: 394 (e.g. any one of SEQ ID NO: 377-380) or a sequence having at least 75%, at least 80%, at least 85%, at least 90%, at least 91%, at least 92%, at least 93%, at least 94%, at least 95%, at least 96%, at least 97%, at least 98%, or at least 99% identity thereto. In some embodiments, the nucleic acid comprising a nucleotide sequence encoding the chimeric C. acnes DsA1/DsA2/PITP/CAMP2 polypeptide comprises a nucleotide sequence that is least 75% identical to any one of SEQ ID NO: 377-380.

In some embodiments, the chimeric C. acnes DsA1/DsA2/PITP/CAMP2 polypeptide comprises a sequence of a chimeric C. acnes DsA1/DsA2/PITP polypeptide as described herein. In some embodiments, the chimeric C. acnes DsA1/DsA2/PITP/CAMP2 polypeptide comprises a sequence of a chimeric C. acnes DsA1/DsA2 polypeptide as described herein.

### Variants of the Polypeptides of the Present Invention

### Mutation of Cysteine Residues

One or more cysteine residues in the polypeptides described herein may be mutated by single amino acid substitutions, e.g. to a serine residue. Cysteine residues are involved in disulphide bridge formation and thus cysteine mutations may limit polypeptide multimerisation.

In some embodiments, the C. acnes DsA1 polypeptide described herein comprises a single amino acid substitution at one or more (e.g. all) positions corresponding to a cysteine residue in a native C. acnes DsA1 polypeptide. In some embodiments, the C. acnes DsA1 polypeptide comprises one or more single amino acid substitutions at positions corresponding to C25 (e.g. C25S), C291 (e.g. C291S or C291M) and/or C293 (e.g. C293S or C293P) of SEQ ID NO: 204.

In some embodiments, the C. acnes DsA2 polypeptide described herein comprises a single amino acid substitution at one or more (e.g. all) positions corresponding to a cysteine residue in a native C. acnes DsA2 polypeptide. In some embodiments, the C. acnes DsA2 polypeptide comprises one or more single amino acid substitutions at positions corresponding to C26 (e.g. C26S) and/or C292 (e.g. C292S) of SEQ ID NO: 205.

In some embodiments, the C. acnes PITP polypeptide described herein comprises a single amino acid substitution at one or more (e.g. all) positions corresponding to a cysteine residue in a native C. acnes PITP polypeptide. In some embodiments, the C. acnes PITP polypeptide comprises one or more single amino acid substitutions at positions corresponding to C200 (e.g. C200S), C371 (e.g. C371S) and/or C429 (e.g. C429S) of SEQ ID NO: 206. In some embodiments, the C. acnes PITP polypeptide comprises a single amino acid substitution at one or both (e.g. both) positions corresponding to C200 (e.g. C200S) and C371 (e.g. C371S) of SEQ ID NO: 206. In preferred embodiments, a C. acnes PITP polypeptide having the sequence of SEQ ID NO: 73 comprises a serine residue at positions 200 and 371. In other embodiments, a C. acnes PITP polypeptide having the sequence of SEQ ID NO: 77 comprises a serine residue at positions 200 and 371.

In some embodiments, the chimeric C. acnes DsA1/DsA2 polypeptide described herein comprises a single amino acid substitution at one or more (e.g. all) positions corresponding to a cysteine residue in a native C. acnes DsA1 polypeptide and/or one or more (e.g. all) positions corresponding to a cysteine residue in a native C. acnes DsA2 polypeptide. In some embodiments, the chimeric C. acnes DsA1/DsA2 polypeptide comprises a single amino acid substitution at one or more (all) positions corresponding to C25 (e.g. C25S), C291 (e.g. C291S or C291M) and/or C293 (e.g. C293S or C293P) of SEQ ID NO: 204. In some embodiments, the chimeric C. acnes DsA1/DsA2 polypeptide comprises one or more (e.g. all three) amino acid substitutions selected from C25S, C291S and/or C293P wherein C25, C291 and/or C293 correspond to the residues in SEQ ID NO: 204. In preferred embodiments, a chimeric C. acnes DsA1/DsA2 polypeptide having the sequence of SEQ ID NO: 70 comprises a serine residue at positions 25 and 291 and a proline residue at position 293. In other embodiments, a chimeric C. acnes DsA1/DsA2 polypeptide having the sequence of SEQ ID NO: 72 comprises a serine residue at positions 25 and 291 and a proline residue at position 293. In other embodiments, a chimeric C. acnes DsA1/DsA2 polypeptide having the sequence of SEQ ID NO: 81 comprises a serine residue at position 18 and a proline residue at position 286. In other embodiments, a chimeric C. acnes DsA1/DsA2 polypeptide having the sequence of SEQ ID NO: 81 comprises a serine residue at position 18, a methionine residue at position 284 and a proline residue at position 286, wherein the residues correspond to positions 25, 291 and 293, respectively, in SEQ ID NO: 204.

In some embodiments, the chimeric C. acnes DsA1/DsA2/PITP polypeptide described herein comprises a single amino acid substitution at one or more (e.g. all) positions corresponding to a cysteine residue in a native C. acnes DsA1 polypeptide, one or more (e.g. all) positions corresponding to a cysteine residue in a native C. acnes DsA2 polypeptide and/or one or more (e.g. all) positions corresponding to a cysteine residue in a native C. acnes PITP polypeptide. In some embodiments, the chimeric C. acnes DsA1/DsA2/PITP polypeptide comprises a single amino acid substitution at one or more positions corresponding to C25 (e.g. C25S), C291 (e.g. C291S or C291M) and/or C293 (e.g. C293S or C293P) of SEQ ID NO: 204. In some embodiments, the chimeric C. acnes DsA1/DsA2/PITP polypeptide comprises one or more (e.g. all three) amino acid substitutions selected from C25S, C291S and/or C293P wherein C25, C291 and/or C293 corresponds to the residues in SEQ ID NO: 204. In some embodiments, a chimeric C. acnes DsA1/DsA2/PITP polypeptide having the sequence of SEQ ID NO: 80 or of SEQ ID NO: 83 (e.g. SEQ ID NO: 83) comprises a serine residue at positions 25 and 291 and a proline residue at position 293. In other embodiments, a chimeric C. acnes DsA1/DsA2/PITP polypeptide having the sequence of SEQ ID NO: 82 comprises a serine residue at position 25 and a proline residue at position 293. In some embodiments, a chimeric C. acnes DsA1/DsA2/PITP polypeptide having the sequence of SEQ ID NO: 82 comprises a serine residue at position 25, a methionine residue at position 291 and a proline residue at position 293. In other embodiments, a chimeric C. acnes DsA1/DsA2/PITP polypeptide having the sequence of SEQ ID NO: 368 comprises a serine residue at position 18, a methionine residue at position 284 and a proline residue at position 286. In some embodiments, a chimeric C. acnes DsA1/DsA2/PITP polypeptide having the sequence of SEQ ID NO: 368 comprises a serine residue at position 18, a methionine residue at position 284 and a proline residue at position 286, wherein the residues correspond to positions 25, 291 and 293, respectively, in SEQ ID NO: 204.

In some embodiments, the chimeric C. acnes DsA1/DsA2/PITP/CAMP2 polypeptide described herein comprises a single amino acid substitution at one or more (e.g. all) positions corresponding to a cysteine residue in a native C. acnes DsA1 polypeptide, one or more (e.g. all) positions corresponding to a cysteine residue in a native C. acnes DsA2 polypeptide and/or one or more (e.g. all) positions corresponding to a cysteine residue in a native C. acnes PITP polypeptide. In some embodiments, the chimeric C. acnes DsA1/DsA2/PITP polypeptide comprises a single amino acid substitution at one or more positions corresponding to C25 (e.g. C25S), C291 (e.g. C291S or C291M) and/or C293 (e.g. C293S or C293P) of SEQ ID NO: 204. In some embodiments, the chimeric C. acnes DsA1/DsA2/PITP/CAMP2 polypeptide comprises one or more (e.g. all three) amino acid substitutions selected from C25S, C291S and/or C293P wherein C25, C291 and/or C293 corresponds to the residues in SEQ ID NO: 204. In some embodiments, a chimeric C. acnes DsA1/DsA2/PITP/CAMP2 polypeptide having the sequence of SEQ ID NO: 373 or of SEQ ID NO: 374 comprises a serine residue at positions 264 and 530 and a proline residue at position 532. In other embodiments, a chimeric C. acnes DsA1/DsA2/PITP/CAMP2 polypeptide having the sequence of SEQ ID NO: 375 comprises a serine residue at positions 185 and 451 and a proline residue at position 453. In other embodiments, a chimeric C. acnes DsA1/DsA2/PITP/CAMP2 polypeptide having the sequence of SEQ ID NO: 376 comprises a serine residue at positions 25 and 291 and a proline residue at position 293.

### Mutation of Glycosylation Sites

Glycosylation may occur in eukaryotic cells but typically, it does not occur in prokaryotic cells. “Glycosylation” as used herein refers to the addition of a saccharide unit to a protein. In particular, N-linked glycosylation is the attachment of glycan to an amide nitrogen of an asparagine (Asn; N) residue in a protein. N-glycosylation can occur at any asparagine residue in a protein that is accessible to and recognised by glycosylating enzymes following translation of the protein, and is most common at accessible asparagines that are part of an NXS/T motif, wherein the second amino acid residue following the asparagine is a serine (Ser; S) or threonine (Thr; T). O-linked glycosylation is the attachment of a glycan to the oxygen atom of serine (Ser) or threonine (Thr) residue in a protein. The process of attachment results in a glycosylated protein. This glycan may be a polysaccharide. A non-human glycosylation pattern can render a polypeptide undesirably reactogenic when used to elicit antibodies. Additionally, glycosylation of a polypeptide that is not normally glycosylated (such as polypeptides described herein, which are polypeptides naturally found in prokaryotic cells or derived from such polypeptides) may alter its immunogenicity. For example, glycosylation can mask important immunogenic epitopes within a protein. Thus, to reduce or eliminate glycosylation, either asparagine residues or serine/threonine residues can be modified, for example, by substitution to another amino acid.

In certain embodiments, one or more of the C. acnes CAMP2 polypeptide, the modified C. acnes CAMP2 polypeptide, the C. acnes DsA1 polypeptide, the C. acnes DsA2 polypeptide, the C. acnes PITP polypeptide, the chimeric C. acnes DsA1/DsA2 polypeptide, the chimeric C. acnes DsA1/DsA2/PITP polypeptide and the chimeric C. acnes DsA1/DsA2/PITP/CAMP2 polypeptide as described herein comprises at least one mutated glycosylation site, preferably at least one mutated N-linked glycosylation site and/or at least one O-linked glycosylation site. In some embodiments, one or more glycosylation sites in one or more of the C. acnes CAMP2 polypeptide, the modified C. acnes CAMP2 polypeptide, the C. acnes DsA1 polypeptide, the C. acnes DsA2 polypeptide, the C. acnes PITP polypeptide, the chimeric C. acnes DsA1/DsA2 polypeptide, the chimeric C. acnes DsA1/DsA2/PITP polypeptide and the chimeric C. acnes DsA1/DsA2/PITP/CAMP2 polypeptide as described herein are removed. The removal of a glycosylation site may decrease glycosylation of the polypeptide. In some embodiments, one or more of the C. acnes CAMP2 polypeptide, the modified C. acnes CAMP2 polypeptide, the C. acnes DsA1 polypeptide, the C. acnes DsA2 polypeptide, the C. acnes PITP polypeptide, the chimeric C. acnes DsA1/DsA2 polypeptide, the chimeric C. acnes DsA1/DsA2/PITP polypeptide and the chimeric C. acnes DsA1/DsA2/PITP/CAMP2 polypeptide as described herein has decreased glycosylation relative to the respective native polypeptide. The removal of glycosylation sites may eliminate glycosylation of the polypeptide.

In certain embodiments, the modification comprises a substitution of one or more of an N, S, and T amino acid (e.g., in an NXS/T sequence motif), wherein X corresponds to any amino acid. In some embodiments, the modification comprises a substitution of one or more serine (Ser) or threonine (Thr) residue(s) in a protein. In some embodiments, an N, S, or T amino acid is substituted with a conservative amino acid substitution. Typically, an N amino acid may be substituted with a Q, S, K or A amino acid.

In some embodiments, the C. acnes CAMP2 polypeptide and/or the modified C. acnes CAMP2 polypeptide described herein comprise a single amino acid substitution at one or more (e.g. all) positions corresponding to a glycosylation site in a native C. acnes CAMP2 polypeptide. A N-glycosylation site in a C. acnes CAMP2 polypeptide and/or a modified C. acnes CAMP2 polypeptide may be present at a position corresponding to residue N166 of SEQ ID NO: 203. A N-glycosylation site may include N166, F167 and S168, with glycosylation at N166, relative to the residue numbering in SEQ ID NO: 203. In some embodiments, a C. acnes CAMP2 polypeptide and/or a modified C. acnes CAMP2 polypeptide of the invention comprise an amino acid substitution at position corresponding to N166 (e.g. N166S), relative to the residue numbering in SEQ ID NO: 203. A C. acnes CAMP2 polypeptide and/or a modified C. acnes CAMP2 polypeptide may comprise the sequence of SEQ ID NO: 50, 51 or 54 with serine at position 166. O-glycosylation sites in a C. acnes CAMP2 polypeptide and/or a modified C. acnes CAMP2 polypeptide may be present at positions corresponding to any S and/or T residues in SEQ ID NO: 203.

In some embodiments, the C. acnes DsA1 polypeptide described herein comprises a single amino acid substitution at one or more (e.g. all) positions corresponding to a glycosylation site in a native C. acnes DsA1 polypeptide. A N-glycosylation site in a C. acnes DsA1 polypeptide may be present at a position corresponding to residue N255 of SEQ ID NO: 204. A N-glycosylation site may include N255, 1256 and T257, with glycosylation at N255, relative to the residue numbering in SEQ ID NO: 204. In some embodiments, a C. acnes DsA1 polypeptide as described herein comprises an amino acid substitution at position corresponding to N255 (e.g. N255Q or N255Q), relative to the residue numbering in SEQ ID NO: 204. O-glycosylation sites in a C. acnes DsA1 polypeptide as described herein may be present at positions corresponding to any S and/or T residues in SEQ ID NO: 204, such as the S292 residue.

In some embodiments, the C. acnes DsA2 polypeptide described herein comprises a single amino acid substitution at one or more (e.g. all) positions corresponding to a glycosylation site in a native C. acnes DsA2 polypeptide. A N-glycosylation site in a C. acnes DsA2 polypeptide may be present at a position corresponding to residue N5 of SEQ ID NO: 205. A N-glycosylation site may include N5, S6 and S7, with glycosylation at N5, relative to the residue numbering in SEQ ID NO: 205. In some embodiments, a C. acnes DsA2 polypeptide as described herein comprises an amino acid substitution at position corresponding to N5 (e.g. N5Q), relative to the residue numbering in SEQ ID NO: 205. A C. acnes DsA2 polypeptide may comprise the sequence of SEQ ID NO: 68 or 69 with glutamine at position 5. O-glycosylation sites in a C. acnes DsA2 polypeptide as described herein may be present at positions corresponding to any S and/or T residues in SEQ ID NO: 205.

In some embodiments, the C. acnes PITP polypeptide described herein comprises a single amino acid substitution at one or more (e.g. all) positions corresponding to a glycosylation site in a native C. acnes PITP polypeptide. N-glycosylation sites in a C. acnes PITP polypeptide may be present at positions corresponding to residues N230, N242, N362 and N373 of SEQ ID NO: 206. N-glycosylation sites may include (i) N230, G231 and S232 (with glycosylation at N230); (ii) N242, G243 and S244 (with glycosylation at N242); (iii) N362, L363 and T364 (with glycosylation at N362); and/or (iv) N373, V374 and T375 (with glycosylation at N373), wherein the amino acid numbering is relative to the residue numbering in SEQ ID NO: 206. In some embodiments, a C. acnes PITP polypeptide of the invention comprises one or more (e.g. all) amino acid substitutions at positions corresponding to N230 (e.g. N230K), S244 (e.g. S244G), N362 (e.g. N362Q) and N373 (e.g. N373S), wherein the amino acid numbering is relative to the residue numbering in SEQ ID NO: 206. In some embodiments, a C. acnes PITP polypeptide as described herein comprises one or more (e.g. all) amino acid substitutions at positions corresponding to N230 (e.g. N230K), N242 (e.g. N242G), N362 (e.g. N362Q) and N373 (e.g. N373S), wherein the amino acid numbering is relative to the residue numbering in SEQ ID NO: 206. O-glycosylation sites in a C. acnes PITP polypeptide may be present at positions corresponding to any S and/or T residues in SEQ ID NO: 206. In some embodiments, a C. acnes PITP polypeptide as described herein comprises the sequence of SEQ ID NO: 76, 77 or 79 with lysine at position 230, glycine at position 244, glutamine at position 362 and serine at position 373.

In some embodiments, the chimeric C. acnes DsA1/DsA2 polypeptide described herein comprises a single amino acid substitution at one or more (e.g. all) positions corresponding to a glycosylation site in a native C. acnes DsA1 polypeptide and/or one or more (e.g. all) positions corresponding to a glycosylation site in a native C. acnes DsA2 polypeptide. A N-glycosylation site in a chimeric C. acnes DsA1/DsA2 polypeptide may be present at a position corresponding to the residue N255 of SEQ ID NO: 70. A N-glycosylation site may include N255, 1256 and T257, with glycosylation at N255, relative to the residue numbering in SEQ ID NO: 70. In some embodiments, the chimeric C. acnes DsA1/DsA2 polypeptide comprises a N255Q or a N255A (e.g. N255Q) substitution in SEQ ID NO: 70. In some embodiments, a chimeric C. acnes DsA1/DsA2 polypeptide of the invention comprises an amino acid substitution at the position corresponding to N255 (e.g. N255Q or N255A), relative to the residue numbering in SEQ ID NO: 70. In some embodiments, the chimeric C. acnes DsA1/DsA2 polypeptide comprises the sequence of SEQ ID NO: 72 with glutamine at position 255. O-glycosylation sites in a chimeric C. acnes DsA1/DsA2 polypeptide may be present at positions corresponding to any S and/or T residues in SEQ ID NO: 70. O-glycosylation sites in a chimeric C. acnes DsA1/DsA2 polypeptide may be present at positions corresponding to residues S291 and/or S292 of SEQ ID NO: 70. An O-glycosylation site may include S291 and S292, with glycosylation at S291 and/or S292, relative to the residue numbering in SEQ ID NO: 70. In some embodiments, the chimeric C. acnes DsA1/DsA2 polypeptide comprises one or more (e.g. all three) substitutions selected from a N255A, a S291M and a S292G. In some embodiments, the chimeric C. acnes DsA1/DsA2 polypeptide comprises one or more (e.g. all three) amino acid substitutions at positions corresponding to N255 (e.g. N255A), S291 (e.g. S291M) and S292 (e.g. S292G), relative to the residue numbering in SEQ ID NO: 70. A chimeric C. acnes DsA1/DsA2 polypeptide may comprise the sequence of SEQ ID NO: 81 with alanine at position 248, methionine at position 284 and glycine at position 285. In some embodiments, the chimeric C. acnes DsA1/DsA2 polypeptide comprises the sequence of SEQ ID NO: 81 with alanine at position 248, methionine at position 284 and glycine at position 285, wherein the residues correspond to positions 255, 291 and 292, respectively, in SEQ ID NO: 70.

In some embodiments, the chimeric C. acnes DsA1/DsA2/PITP polypeptide described herein comprises a single amino acid substitution at one or more (e.g. all) positions corresponding to a glycosylation site in a native C. acnes DsA1 polypeptide, one or more (e.g. all) positions corresponding to a glycosylation site in a native C. acnes DsA2 polypeptide and/or one or more (e.g. all) positions corresponding to a glycosylation site in a native C. acnes PITP polypeptide. A N-glycosylation site in a chimeric C. acnes DsA1/DsA2/PITP polypeptide may be present at a position corresponding to the residue N255, relative to the residue numbering in SEQ ID NO: 80 or 83. A N-glycosylation site may include N255, 1256 and T257, with glycosylation at N255, relative to the residue numbering in SEQ ID NO: 80 or 83. In some embodiments, the chimeric C. acnes DsA1/DsA2/PITP polypeptide comprises a N255Q or a N255A (e.g. N255A) substitution in SEQ ID NO: 80 or 83. In some embodiments, a chimeric C. acnes DsA1/DsA2/PITP polypeptide of the invention comprises an amino acid substitution at the position corresponding to N255 (e.g. N255Q or N255A), relative to the residue numbering in SEQ ID NO: 80 or 83. O-glycosylation sites in a chimeric C. acnes DsA1/DsA2/PITP polypeptide may be present at positions corresponding to any S and/or T residues in SEQ ID NO: 80 or 83. O-glycosylation sites in a chimeric C. acnes DsA1/DsA2/PITP polypeptide may be present at one or more (e.g. all) positions corresponding to residues S291, S292, T299, T301, T303, T305, T425, S428 and T436 of SEQ ID NO: 80 or 83. In some embodiments, a chimeric C. acnes DsA1/DsA2/PITP polypeptide as described herein comprises the substitution of the PTPTPTPT region located between positions 298 and 305 of SEQ ID NO: 80 or 83, with a GGGGG linker. In some embodiments, the chimeric C. acnes DsA1/DsA2/PITP polypeptide comprises one or more (e.g. all) substitutions selected from a S291M, S292G, P298G, T299G, P300G, T301G, P302G, T425G, S428G and T436G. In some embodiments, the chimeric C. acnes DsA1/DsA2/PITP polypeptide comprises one or more (e.g. all) substitutions selected from a S291M, S292G, T425G, S428G and T436G, relative to the residue numbering in SEQ ID NO: 80 or 83. In some embodiments, the chimeric C. acnes DsA1/DsA2/PITP polypeptide comprises one or more (e.g. all) amino acid substitutions at positions corresponding to S291 (e.g. S291M), S292 (e.g. S292G), T425 (e.g. T425G), S428 (e.g. S428G) and T436 (e.g. T436G), relative to the residue numbering in SEQ ID NO: 80 or 83. A chimeric C. acnes DsA1/DsA2/PITP polypeptide may comprise the sequence of SEQ ID NO: 82 with methionine at position 291 and glycine at positions 292, 299, 301, 422, 425 and 433. In some embodiments, the chimeric C. acnes DsA1/DsA2/PITP polypeptide may comprise the sequence of SEQ ID NO: 82 with methionine at position 291 and glycine at positions 292, 298, 299, 300, 301, 302, 422, 425 and 433. In some embodiments, the chimeric C. acnes DsA1/DsA2/PITP polypeptide may comprise the sequence of SEQ ID NO: 368 with alanine at position 248, methionine at position 284 and glycine at positions 285, 291, 292, 293, 294, 295, 415, 418 and 426.

In some embodiments, the chimeric C. acnes DsA1/DsA2/PITP/CAMP2 polypeptide described herein comprises a single amino acid substitution at one or more (e.g. all) positions corresponding to a glycosylation site in a native C. acnes DsA1 polypeptide, one or more (e.g. all) positions corresponding to a glycosylation site in a native C. acnes DsA2 polypeptide, one or more (e.g. all) positions corresponding to a glycosylation site in a native C. acnes PITP polypeptide and/or one or more (e.g. all) positions corresponding to a glycosylation site in a native C. acnes CAMP2 polypeptide.

As described in the section above “Chimeric DsA1/DsA2/PITP/CAMP2”, a chimeric C. acnes DsA1/DsA2/PITP/CAMP2 polypeptide may comprise a C. acnes CAMP2 polypeptide or immunogenic fragment thereof. A N-glycosylation site in a chimeric C. acnes DsA1/DsA2/PITP/CAMP2 polypeptide may be present at a position corresponding to residue N166 of SEQ ID NO: 203 (i.e. corresponding to N194 of SEQ ID NO: 202), if that residue is present in the chimeric C. acnes DsA1/DsA2/PITP/CAMP2 polypeptide. A N-glycosylation site in a chimeric C. acnes DsA1/DsA2/PITP/CAMP2 polypeptide may be present at position N166, F167 and S168, with glycosylation at N166, relative to the residue numbering in SEQ ID NO: 203 (i.e., corresponding to position N194, F195 and S196, with glycosylation at N194, relative to the residue numbering in SEQ ID NO: 202), if those residues are present in the chimeric C. acnes DsA1/DsA2/PITP/CAMP2 polypeptide. In some embodiments, a C. acnes DsA1/DsA2/PITP/CAMP2 polypeptide of the invention comprises an amino acid substitution at position corresponding to N166 (e.g. N166S), relative to the residue numbering in SEQ ID NO: 203 (i.e., corresponding to N194 (e.g. N194S), relative to the residue numbering in SEQ ID NO: 202), if present in the polypeptide. O-glycosylation sites in a C. acnes DsA1/DsA2/PITP/CAMP2 polypeptide may be present at positions corresponding to any S and/or T residues in SEQ ID NO: 203 (i.e., corresponding to any S and/or T residues in residues 29-267 of SEQ ID NO: 202), if present in the chimeric C. acnes DsA1/DsA2/PITP/CAMP2 polypeptide.

As described in the section above “Chimeric DsA1/DsA2/PITP/CAMP2”, a chimeric C. acnes DsA1/DsA2/PITP/CAMP2 polypeptide may comprise SEQ ID NO: 80, 82 or 83. A N-glycosylation site in a chimeric C. acnes DsA1/DsA2/PITP/CAMP2 polypeptide may be present at a position corresponding to residue N255, relative to the residue numbering in SEQ ID NO: 80 or 83. A N-glycosylation site in a chimeric C. acnes DsA1/DsA2/PITP/CAMP2 polypeptide may include N255, 1256 and T257, with glycosylation at N255, relative to the residue numbering in SEQ ID NO: 80 or 83. In some embodiments, a chimeric C. acnes DsA1/DsA2/PITP/CAMP2 polypeptide of the invention comprises an amino acid substitution at the position corresponding to N255 (e.g. N255Q or N255A), relative to the residue numbering in SEQ ID NO: 80 or 83. O-glycosylation sites in a chimeric C. acnes DsA1/DsA2/PITP/CAMP2 polypeptide may be present at positions corresponding to any S and/or T residues in SEQ ID NO: 80 or 83. O-glycosylation sites in a chimeric C. acnes DsA1/DsA2/PITP/CAMP2 polypeptide may be present at one or more (e.g. all) positions corresponding to residues S291, S292, T299, T301, T303, T305, T425, S428 and T436 of SEQ ID NO: 80 or 83. In some embodiments, a chimeric C. acnes DsA1/DsA2/PITP/CAMP2 polypeptide as described herein comprises the substitution of the PTPTPTPT region located between positions 298 and 305 of SEQ ID NO: 80 or 83, with a GGGGG linker. In some embodiments, the chimeric C. acnes DsA1/DsA2/PITP/CAMP2 polypeptide comprises one or more (e.g. all) substitutions selected from a S291M, S292G, P298G, T299G, P300G, T301G, P302G, T425G, S428G and T436G. In some embodiments, the chimeric C. acnes DsA1/DsA2/PITP/CAMP2 polypeptide comprises one or more (e.g. all) substitutions selected from a S291M, S292G, T425G, S428G and T436G, relative to the residue numbering in SEQ ID NO: 80 or 83. In some embodiments, the chimeric C. acnes DsA1/DsA2/PITP/CAMP2 polypeptide comprises one or more (e.g. all) amino acid substitutions at positions corresponding to S291 (e.g. S291M), S292 (e.g. S292G), T425 (e.g. T425G), S428 (e.g. S428G) and T436 (e.g. T436G), relative to the residue numbering in SEQ ID NO: 80 or 83. A chimeric C. acnes DsA1/DsA2/PITP/CAMP2 polypeptide may comprise the sequence of SEQ ID NO: 82 with methionine at position 291 and glycine at positions 292, 299, 301, 422, 425 and 433. In some embodiments, the chimeric C. acnes DsA1/DsA2/PITP/CAMP2 polypeptide may comprise the sequence of SEQ ID NO: 82 with methionine at position 291 and glycine at positions 292, 298, 299, 300, 301, 302, 422, 425 and 433.

### Secretion Signal Peptide (SS) Sequences

The C. acnes CAMP2 polypeptide, the C. acnes DsA1 polypeptide, the C. acnes DsA2 polypeptide, the C. acnes PITP polypeptide, the chimeric C. acnes DsA1/DsA2 polypeptide, the chimeric C. acnes DsA1/DsA2/PITP polypeptide and/or the chimeric C. acnes DsA1/DsA2/PITP/CAMP2 polypeptide as described herein may comprise a secretion signal peptide (SS) sequence. The secretion signal peptide may be cleaved in post-translation processing of the C. acnes polypeptides described herein. The mature form of the C. acnes polypeptide may therefore not comprise the secretion signal peptide sequence. However, a nucleotide sequence encoding a secretion signal peptide sequence may be present in nucleic acids described herein encoding the C. acnes polypeptides described herein.

The C. acnes CAMP2 polypeptide, the C. acnes DsA1 polypeptide, the C. acnes DsA2 polypeptide, the C. acnes PITP polypeptide, the chimeric C. acnes DsA1/DsA2 polypeptide, the chimeric C. acnes DsA1/DsA2/PITP polypeptide and/or the chimeric C. acnes DsA1/DsA2/PITP/CAMP2 polypeptide as described herein may comprise a secretion signal peptide sequence of the respective native C. acnes polypeptide. This may be advantageous if the C. acnes polypeptides are expressed in a prokaryotic cell as recombinant proteins.

In some embodiments, the C. acnes CAMP2 polypeptide, the C. acnes DsA1 polypeptide, the C. acnes DsA2 polypeptide, the C. acnes PITP polypeptide, the chimeric C. acnes DsA1/DsA2 polypeptide, the chimeric C acnes DsA1/DsA2/PITP polypeptide and/or the chimeric C. acnes DsA1/DsA2/PITP/CAMP2 polypeptide as described herein may comprise viral or eukaryotic (e.g. human) secretion signal peptide sequences. The use of viral or eukaryotic secretion signal peptide sequences attached to a polypeptide described herein may offer numerous advantages for immunogenic compositions. When expressed from an mRNA, especially in a eukaryotic cell, a polypeptide of the invention comprising a SS sequence may have increased extracellular expression relative to the polypeptide without the SS sequence. The increased extracellular expression may promote higher immunogenicity and by extension, better vaccine efficacy.

Viral SS sequences may be found in publicly accessible databases (e.g., the NCBI or UniProt databases) which include an annotated viral polypeptide sequence and identify the start and end position of an experimentally validated SS.

In certain embodiments, the SS sequence as well as the location of the SS sequence cleavage site for a given known input polypeptide sequence may be predicted by using the SignalP algorithm. The SignalP algorithm (and more particularly SignalP v6.0) is described in further detail in Armenteros et al. (Nature Biotechnology. 37:420-423. 2019), Teufel et al. (Nature Biotechnology. 40:1023-1025. 2022), and services.healthtech.dtu.dk/services/SignalP-6.0/, each of which is incorporated herein by reference in their entirety. The strength of the prediction is assessed based on a cumulative rank score that considers the likelihood of detecting canonical features of the signal sequence (SS likelihood score) and the probability of cleavage at the cleavage site (cleavage probability score). In certain embodiments, the viral secretion signal peptide has a SignalP cleavage probability score of at least 0.8, at least 0.85, at least 0.90 or at least 0.95, as determined using SignalP 6.0. In some embodiments, the viral secretion signal peptide has a SignalP signal peptide likelihood score of at least 0.8, at least 0.85, at least 0.90 or at least 0.95, as determined using SignalP 6.0.

In certain embodiments, the SS sequence is a viral SS sequence. In certain embodiments, the viral secretion signal peptide sequence is derived from a viral sequence in a virus able to infect humans. The phrase “influenza”, “SARS CoV-2”, “varicella-zoster virus (VZV)”, “measles”, “rubella”, “rabies,” “Ebola,” and “smallpox” preceding the phrase “secretion signal peptide sequence” indicates that the secretion signal peptide was derived from the virus corresponding to that name.

In certain embodiments, the viral secretion signal peptide is derived from a viral sequence selected from the group consisting of: an influenza secretion signal peptide sequence, a SARS CoV-2 secretion signal peptide sequence, a varicella-zoster virus (VZV) secretion signal peptide sequence, a measles secretion signal peptide sequence, a rubella secretion signal peptide sequence, a mumps secretion signal peptide sequence, an Ebola secretion signal peptide sequence, a rabies secretion signal peptide sequence, and a smallpox secretion signal peptide sequence. These particular signal peptides are derived from viral sequences in viruses which have been administered to humans as vaccines (live-attenuated, inactivated or mRNA), with demonstrated strong safety profiles.

In certain embodiments, the viral secretion signal peptide is selected from the group consisting of: an influenza hemagglutinin (HA) secretion signal peptide sequence, a SARS CoV-2 spike secretion signal peptide sequence, a VZV gB secretion signal peptide sequence, a VZV gE secretion signal peptide sequence, a VZV gI secretion signal peptide sequence, a VZV gK secretion signal peptide sequence, a measles F-protein secretion signal peptide sequence, a rubella E1 protein secretion signal peptide sequence, a rubella E2 protein secretion signal peptide sequence, a mumps F-protein secretion signal peptide sequence, an Ebola GP protein secretion signal peptide sequence, a rabies virus glycoprotein (Rabies G) secretion signal peptide sequence, and a smallpox 6 kDa IC protein secretion signal peptide sequence.

In certain embodiments, the viral secretion signal peptide comprises an HA secretion signal peptide sequence from influenza A or influenza B, preferably from influenza A.

Exemplary viral secretion signal peptide amino acid sequences of the disclosure are shown below in Table 2. Exemplary viral secretion signal peptide amino acid sequences derived from Influenza A or B of the disclosure are shown below in Table 3.

TABLE 2
Viral Secretion Signal Peptide (SS)
Amino Acid Sequences

| NAME | | | |
|---|---|---|---|
| OF THE | | | SEQUENCE OF |
| PROTEIN | ORGANISM | STRAIN | THE SS |
| HA (HIN1) | Influenza | A/New | MKAKLLVLLCTFTATYA |
| | virus | Caledonia/ | (SEQ ID NO: 210) |
| | | 20/1999 | |
| HA | Influenza | A/California/ | MKAILVVLLYTFATANA |
| (H1N1pdm) | virus | 7/2009 | (SEQ ID NO: 211) |
| HA (H3N2) | Influenza | A/Moscow/10/ | MKTIIALSYILCLVFA |
| | virus | 1999 | (SEQ ID NO: 212) |
| HAB | Influenza | B/Phuket/ | MKAIIVLLMVVTSNA |
| | virus | 3073/2013 | (SEQ ID NO: 213) |
| Spike | SARS | Wuhan-1 | MFVFLVLLPLVS |
| | CoV-2 | | (SEQ ID NO: 214) |
| Spike | SARS | Wuhan-1 (long | MFLLTTKRTMFVFLVLL |
| | CoV-2 | version) | PLVS |
| | | | (SEQ ID NO: 215) |
| gB | VZV | Oka strain | MSPCGYYSKWRNRDRPE |
| | | | YRRNLRFRRFFSSIHPN |
| | | | AAAGSGFNGPGVFITSV |
| | | | TGVWLCFLCIFSMFVTA |
| | | | VVS |
| | | | (SEQ ID NO: 216) |
| gE | VZV | Oka strain | MGTVNKPVVGVLMGFGI |
| | | | ITGTLRITNPVRA |
| | | | (SEQ ID NO: 217) |
| gI | VZV | Oka strain | MFLIQCLISAVIFYIQV |
| | | | TNA |
| | | | (SEQ ID NO: 218) |
| gK | VZV | Oka strain | MQALGIKTEHFIIMCLL |
| | | | SGHA |
| | | | (SEQ ID NO: 219) |
| F | Measles | Edmonston- | MGLKVNVSAIFMAVLLT |
| | | Zagreb | LQTPTG |
| | | strain | (SEQ ID NO: 220) |
| E1 | Rubella | RA27/3 strain | MGAAAALTAVVLQGYNP |
| | | | PAYG |
| | | | (SEQ ID NO: 221) |
| E2 | Rubella | RA27/3 strain | MGAPQAFLAGLLLAAVA |
| | | | VGTARA |
| | | | (SEQ ID NO: 222) |
| F | Mumps | Miyahara | MKVFLVTCLGFAVFSSS |
| | | strain | VC |
| | | | (SEQ ID NO: 223) |
| GP | Ebola | Mayinga-76 | MGVTGILQLPRDRFKRT |
| | virus | strain | SFFLWVIILFQRTFS |
| | | | (SEQ ID NO: 224) |
| 6kDa IC | Smallpox | Germany 91-3 | MRSLIIFLLFPSIIYS |
| | | strain | (SEQ ID NO: 225) |
| Rabies G | Rabies | Rabies | MVPQALLFVPLLVFPLC |
| | | Pasteur | FG |
| | | strain | (SEQ ID NO: 226) |

TABLE 3
Influenza A and B Specific Viral
Secretion Signal Peptide (SS)
Amino Acid Sequences

| STRAIN NAME OR ID | SEQUENCE OF THE SS |
|---|---|
| A/Beijing/262/95 | MKAKLLVLLCTFTATYA |
| (HIN1)-like virus | (SEQ ID NO: 210) |
| A/Brisbane/02/2018 | MKAILVVLLYTFTTANA |
| (H1N1)pdm09-like virus | (SEQ ID NO: 227) |
| A/Brisbane/59/2007 | MKVKLLVLLCTFTATYA |
| (HIN1)-like virus | (SEQ ID NO: 228) |
| A/California/7/2009 | MKAILVVLLYTFATANA |
| (HIN1)-like virus | (SEQ ID NO: 211) |
| A/Guangdong- | MKAILVVLLYTFTTANA |
| Maonan/SWL1536/2019 | (SEQ ID NO: 227) |
| (HIN1)pdm09-like virus | |
| A/Hawaii/70/2019 | MKAILVVLLYTFTTANA |
| (H1N1)pdm09-like virus | (SEQ ID NO: 227) |
| A/Michigan/45/2015 | MKAILVVLLYTFTTANA |
| (H1N1)pdm09-like virus | (SEQ ID NO: 227) |
| A/New Caledonia/20/99 | MKAKLLVLLCTFTATYA |
| (HIN1)-like virus | (SEQ ID NO: 210) |
| A/Solomon | MKVKLLVLLCTFTATYA |
| Islands/3/2006 (HIN1)- | (SEQ ID NO: 228) |
| like virus | |
| A/Sydney/5/2021 | MKAILVVMLYTFTTANA |
| (H1N1)pdm09-like virus | (SEQ ID NO: 229) |
| A/Victoria/2570/2019 | MKAILVVMLYTFTTANA |
| (HIN1)pdm09-like virus | (SEQ ID NO: 229) |
| A/Victoria/4897/2022 | MKAILVVMLYTFTTANA |
| (HIN1)pdm09-like virus | (SEQ ID NO: 229) |
| A/Wisconsin/588/2019 | MKAILVVMLYTFTTANA |
| (H1N1)pdm09-like virus | (SEQ ID NO: 229) |
| A/Wisconsin/67/2022 | MKAILVVMLYTFTTANA |
| (HIN1)pdm09-like virus | (SEQ ID NO: 229) |
| H1N1 CONSENSUS | MKAILVVLLYTFTTANA |
| SEQ #1 (without | (SEQ ID NO: 227) |
| substitutions) | |
| A/Brisbane/10/2007 | MKTIIALSYILCLVFT |
| (H3N2)-like virus | (SEQ ID NO: 230) |
| A/California/7/2004 | MKTIIALSYILCLVFA |
| (H3N2)-like virus | (SEQ ID NO: 212) |
| A/Cambodia/e0826360/2 | MKTIIALSYILCLVFA |
| 020 (H3N2)-like virus | (SEQ ID NO: 212) |
| A/Darwin/6/2021 | MKTIIALSNILCLVFA |
| (H3N2)-like virus | (SEQ ID NO: 231) |
| A/Darwin/9/2021 | MKTIIALSNILCLVFA |
| (H3N2)-like virus | (SEQ ID NO: 231) |
| A/Fujian/411/2002 | MKTIIALSYILCLVFA |
| (H3N2)-like virus | (SEQ ID NO: 212) |
| A/Hong Kong/2671/2019 | MKAIIALSNILCLVFA |
| (H3N2)-like virus | (SEQ ID NO: 232) |
| A/Hong Kong/45/2019 | MKAIIALSNILCLVFA |
| (H3N2)-like virus | (SEQ ID NO: 232) |
| A/Hong Kong/4801/2014 | MKTIIALSYILCLVFA |
| (H3N2)-like virus | (SEQ ID NO: 212) |
| A/Kansas/14/2017 | MKTIIALSCILCLVFA |
| (H3N2)-like virus | (SEQ ID NO: 233) |
| A/Moscow/10/99 | MKTIIALSYILCLVFA |
| (H3N2)-like virus | (SEQ ID NO: 212) |
| A/Perth/16/2009 (H3N2)- | MKTIIALSYILCLVFA |
| like virus | (SEQ ID NO: 212) |
| A/Singapore/INFIMH- | MKTIIALSYILCLVFA |
| 16-0019/2016 (H3N2)- | (SEQ ID NO: 212) |
| like virus | |
| A/South | MKTIIALSYILCLVFA |
| Australia/34/2019 | (SEQ ID NO: 212) |
| (H3N2)-like virus | |
| A/Switzerland/8060/2017 | MKTIIALSYILCLVFA |
| (H3N2)-like virus | (SEQ ID NO: 212) |
| A/Switzerland/9715293/ | MKTIIALSYILCLVFA |
| 2013 (H3N2)-like virus | (SEQ ID NO: 212) |
| A/Sydney/5/97 (H3N2)- | MKTIIALSYILCLVFA |
| like virus | (SEQ ID NO: 212) |
| A/Texas/50/2012 | MKTIIALSYILCLVFA |
| (H3N2)-like virus | (SEQ ID NO: 212) |
| A/Victoria/361/2011 | MKTIIALSHILCLVFA |
| (H3N2)-like virus | (SEQ ID NO: 234) |
| A/Wellington/1/2004 | MKTIIALSYILCLVFA |
| (H3N2)-like virus | (SEQ ID NO: 212) |
| A/Wisconsin/67/2005 | MKTIIALSYILCLVFA |
| (H3N2)-like virus | (SEQ ID NO: 212) |
| H3N2 CONSENSUS | MKTIIALSYILCLVFA |
| SEQ #1 (without | (SEQ ID NO: 212) |
| substitutions) | |
| B/Austria/1359417/2021 | MKAIIVLLMVVTSNA |
| (B/Victoria lineage)- | (SEQ ID NO: 213) |
| like | |
| B/Brisbane/60/2008-like | MKAIIVLLMVVTSNA |
| virus | (SEQ ID NO: 213) |
| B/Colorado/06/2017-like | MKAIIVLLMVVTSSA |
| virus (B/Victoria/2/87 | (SEQ ID NO: 235) |
| lineage) | |
| B/Hong Kong/330/2001- | MKAIIVLLMVVTSNA |
| like virus | (SEQ ID NO: 213) |
| B/Malaysia/2506/2004- | MKAIVLLMVVTSNA |
| like virus | (SEQ ID NO: 213) |
| B/Washington/02/2019 | MKAIIVLLMVVTSNA |
| (B/Victoria lineage)- | (SEQ ID NO: 213) |
| like virus | |
| B/Beijing/184/93-like | MKAIIVLLMVVTSNA |
| virus | (SEQ ID NO: 213) |
| B/Florida/4/2006-like | MKAIIVLLMVVTSNA |
| virus | (SEQ ID NO: 213) |
| B/Massachusetts/2/2012- | MKAIIVLLMVVTSNA |
| like virus | (SEQ ID NO: 213) |
| B/Phuket/3073/2013-like | MKAIIVLLMVVTSNA |
| virus | (SEQ ID NO: 213) |
| B/Sichuan/379/99-like | MEAIIVLLMVVTSNA |
| virus | (SEQ ID NO: 236) |
| INFLUENZA B | MKAIIVLLMVVTSNA |
| VICTORIA/YAMAGAT | (SEQ ID NO: 213) |
| A CONSENSUS | |
| SEQUENCE (without | |
| substitutions) | |
| H1N1 CONSENSUS | MKX₁X₂LX₃VX₄LX₅T |
| SEQ #2 (with | FX₆X₇X₈X₉A |
| substitutions) | X₁ is |
| | selected from A |
| | and V; X₂ is |
| | selected from I |
| | and K; X₃ is |
| | selected from V |
| | and L; X₄ is |
| | selected from L |
| | and M; X₅ is |
| | selected from Y |
| | and C; X₆ is |
| | selected from T |
| | and A; X₇ is |
| | selected from T |
| | and A; X₈ is |
| | selected from A |
| | and T; and X₉ |
| | is selected |
| | from N and Y |
| | (SEQ ID NO: 237) |
| H3N2 CONSENSUS | MKX₁IIALSX₂ILCLV |
| SEQ #2 (with | FX₃ |
| substitutions) | X₁ is selected |
| | from T and A; |
| | X₂ is selected |
| | from Y, N, C, |
| | and H; and |
| | X₃ is selected |
| | from T and A |
| | (SEQ ID NO: 238) |
| INFLUENZA B | MKAIIVLLMVVTSX₁A |
| VICTORIA | X₁ is selected |
| CONSENSUS SEQ (with | from S and N |
| substitutions) | (SEQ ID NO: 239) |
| INFLUENZA B | MX₁AIIVLLMVVTSNA |
| YAMAGATA | X₁ is selected |
| CONSENSUS SEQ (with | from K and E |
| substitutions) | (SEQ ID NO: 240) |

The secretion signal peptide sequence may be positioned at the N terminus or the C terminus (e.g. at the N terminus) of a polypeptide described herein.

In certain embodiments, the SS amino acid sequence is encoded by a codon-optimized polynucleotide sequence.

In certain embodiments, the viral secretion signal peptide is derived from a viral sequence in a virus able to infect humans.

In certain embodiments, the viral secretion signal peptide is derived from a viral sequence selected from the group consisting of: an influenza secretion signal peptide sequence, and a non-influenza secretion signal peptide sequence selected from the group consisting of a SARS CoV-2 secretion signal peptide sequence, a varicella-zoster virus (VZV) secretion signal peptide sequence, a measles secretion signal peptide sequence, a rubella secretion signal peptide sequence, a mumps secretion signal peptide sequence, an Ebola secretion signal peptide sequence, a smallpox secretion signal peptide sequence, and a rabies secretion signal peptide sequence.

In certain embodiments, the viral secretion signal peptide is selected from the group consisting of: an influenza hemagglutinin (HA) secretion signal peptide sequence, a SARS CoV-2 spike secretion signal peptide sequence, a VZV gB secretion signal peptide sequence, a VZV gE secretion signal peptide sequence, a VZV gI secretion signal peptide sequence, a VZV gK secretion signal peptide sequence, a measles F-protein secretion signal peptide sequence, a rubella E1 protein secretion signal peptide sequence, a rubella E2 protein secretion signal peptide sequence, a mumps F-protein secretion signal peptide sequence, an Ebola GP protein secretion signal peptide sequence, a smallpox 6 kDa IC protein secretion signal peptide sequence, and a rabies G protein secretion signal peptide sequence, preferably wherein the viral secretion signal peptide comprises an HA secretion signal peptide sequence from influenza A or influenza B, more preferably from influenza A.

In certain embodiments, the HA secretion signal peptide sequence comprises an amino acid sequence MKX1X2LX3VX4LX5TFX6X7X8X9A (SEQ ID NO: 237) wherein X1 is selected from A and V; X2 is selected from I and K; X3 is selected from V and L; X4 is selected from L and M; X5 is selected from Y and C; X6 is selected from T and A X7 is selected from T and A; X8 is selected from A and T; and X9 is selected from N and Y.

In certain embodiments, the HA secretion signal peptide sequence comprises an amino acid sequence selected from MKAKLLVLLCTFTATYA (SEQ ID NO: 210), MKAILVVLLYTFTTANA (SEQ ID NO: 227), MKVKLLVLLCTFTATYA (SEQ ID NO: 228), MKAILVVLLYTFATANA (SEQ ID NO: 211), and MKAILVVMLYTFTTANA (SEQ ID NO: 229).

In certain embodiments, the HA secretion signal peptide sequence comprises an amino acid sequence MKX1IIALSX2ILCLVFX3 (SEQ ID NO: 238) wherein X1 is selected from T and A; X2 is selected from Y, N, C, and H; and X3 is selected from T and A.

In certain embodiments, the HA secretion signal peptide sequence comprises an amino acid sequence selected from MKTIIALSYILCLVFT (SEQ ID NO: 230), MKTIIALSYILCLVFA (SEQ ID NO: 212), MKTIIALSNILCLVFA (SEQ ID NO: 231), MKAIIALSNILCLVFA (SEQ ID NO: 232), MKTIIALSCILCLVFA (SEQ ID NO: 233) and MKTIIALSHILCLVFA (SEQ ID NO: 234).

In certain embodiments, the HA secretion signal peptide sequence comprises an amino acid sequence MKAIIVLLMVVTSXIA (SEQ ID NO: 239) wherein X1 is selected from S and N.

In certain embodiments, the HA secretion signal peptide sequence comprises an amino acid sequence MX1AIIVLLMVVTSNA (SEQ ID NO: 240) wherein X1 is selected from K and E.

In certain embodiments, the HA secretion signal peptide sequence comprises an amino acid sequence selected from MKAIIVLLMVVTSNA (SEQ ID NO: 213), MKAIIVLLMVVISSA (SEQ ID NO: 235), and MEAIIVLLMVVTSNA (SEQ ID NO: 236).

In certain embodiments, the viral secretion signal peptide comprises an amino acid sequence selected from the group consisting of: MKAKLLVLLCTFTATYA (SEQ ID NO: 210); MKAILVVLLYTFATANA (SEQ ID NO: 211); MKTIIALSYILCLVFA (SEQ ID NO: 212); MKAIIVLLMVVTSNA (SEQ ID NO: 213); MFVFLVLLPLVS (SEQ ID NO: 214); MFLLTTKRTMFVFLVLLPLVS ID (SEQ NO: 215) MSPCGYYSKWRNRDRPEYRRNLRFRRFFSSIHPNAAAGSGFNGPGVFITSVTGVWLCFLCIFS MFVTAVVS (SEQ ID NO: 216); MGTVNKPVVGVLMGFGIITGTLRITNPVRA (SEQ ID NO: 217); MFLIQCLISAVIFYIQVTNA (SEQ ID NO: 218); MQALGIKTEHFIIMCLLSGHA (SEQ ID NO: 219); MGLKVNVSAIFMAVLLTLQTPTG (SEQ ID NO: 220); MGAAAALTAVVLQGYNPPAYG (SEQ ID NO: 221); MGAPQAFLAGLLLAAVAVGTARA (SEQ ID NO: 222); MKVFLVTCLGFAVFSSSVC (SEQ ID NO: 223); MGVTGILQLPRDRFKRTSFFLWVIILFQRTFS (SEQ ID NO: 224); MRSLIIFLLFPSIIYS (SEQ ID NO: 225); and MVPQALLFVPLLVFPLCFG (SEQ ID NO: 226).

In certain embodiments, the viral secretion signal peptide comprises an amino acid sequence of

(SEQ ID NO: 210)
MKAKLLVLLCTFTATYA.

In certain embodiments, the viral secretion signal peptide is positioned at the N-terminus of a polypeptide disclosed herein.

In certain embodiments, the viral secretion signal peptide is positioned at the C-terminus of a polypeptide disclosed herein.

In certain embodiments, the viral secretion signal peptide is attached to a polypeptide disclosed herein with a linker.

### Heterologous Transmembrane Domains (TMBs)

The C. acnes CAMP2 polypeptide, the C. acnes DsA1 polypeptide, the C. acnes DsA2 polypeptide, the C. acnes PITP polypeptide, the chimeric C. acnes DsA1/DsA2 polypeptide, the chimeric C. acnes DsA1/DsA2/PITP polypeptide and/or the chimeric C. acnes DsA1/DsA2/PITP/CAMP2 polypeptide as described herein may comprise a transmembrane domain (TMB), more particularly a heterologous (non-native) TMB. The inclusion of a heterologous TMB may be advantageous as this will localise the antigen to the cell membrane. This may reduce antigen intracellular localisation and further promote higher immunogenicity relative to the antigen without the TMB sequence.

The C. acnes CAMP2 polypeptide, the C. acnes DsA1 polypeptide, the C. acnes DsA2 polypeptide, the C. acnes PITP polypeptide, the chimeric C. acnes DsA1/DsA2 polypeptide, the chimeric C. acnes DsA1/DsA2/PITP polypeptide and/or the chimeric C. acnes DsA1/DsA2/PITP/CAMP2 polypeptide comprising a heterologous transmembrane domain may also comprise a secretion signal peptide sequence. Typically, a nucleic acid described herein encoding the chimeric C. acnes CAMP2 polypeptide that comprises a heterologous transmembrane domain also comprises a nucleotide sequence encoding a secretion signal peptide sequence. Typically, a nucleic acid described herein encoding the C. acnes DsA1 polypeptide that comprises a heterologous transmembrane domain also comprises a nucleotide sequence encoding a secretion signal peptide sequence. Typically, a nucleic acid described herein encoding the C. acnes DsA2 polypeptide that comprises a heterologous transmembrane domain also comprises a nucleotide sequence encoding a secretion signal peptide sequence. Typically, a nucleic acid described herein encoding the C. acnes PITP polypeptide that comprises a heterologous transmembrane domain also comprises a nucleotide sequence encoding a secretion signal peptide sequence. Typically, a nucleic acid described herein encoding the chimeric C. acnes DsA1/DsA2 polypeptide that comprises a heterologous transmembrane domain also comprises a nucleotide sequence encoding a secretion signal peptide sequence. Typically, a nucleic acid described herein encoding the chimeric C. acnes DsA1/DsA2/PITP polypeptide that comprises a heterologous transmembrane domain also comprises a nucleotide sequence encoding a secretion signal peptide sequence. Typically, a nucleic acid described herein encoding the chimeric C. acnes DsA1/DsA2/PITP/CAMP2 polypeptide that comprises a heterologous transmembrane domain also comprises a nucleotide sequence encoding a secretion signal peptide sequence.

The TMB may be from any known TMB in the art, including but not limited to, TMBs from eukaryotic transmembrane proteins (e.g., mammalian transmembrane proteins, such as human transmembrane proteins), TMBs from prokaryotic transmembrane proteins, and TMBs from viral transmembrane proteins. TMBs may further be identified through in silico prediction algorithms, for example, in the TMHMM prediction method described in Krogh et al. (J Mol Biol. 305 (3): 567-580. 2001) and services.healthtech.dtu.dk/services/TMHMM-2.0/, each of which is incorporated herein by reference in their entirety. Some features of TMBs are described in further detail in Albers et al. (Chapter 2-cell membrane structures and functions. Basic Neurochemistry eighth edition. Pages 26-39. 2012), incorporated herein by reference. TMBs are typically, but not exclusively, comprised predominantly of nonpolar (hydrophobic) amino acid residues and may traverse a lipid bilayer once or several times. The skilled person knows well methods to determine the hydrophobicity of an amino acid. See Simm et al. (2016), Biol Res., 49 (1): 31; Wimlet and White (1996), Nat Struct Biol., 3 (10): 842-848; blanco.biomol.uci.edu/hydrophobicity_scales.html; and cgl.ucsf.edu/chimera/docs/UsersGuide/midas/hydrophob.html.

The TMBs usually comprise alpha helices, each helix containing 18-21 amino acids, which is sufficient to span the lipid bilayer. Accordingly, in certain embodiments, the transmembrane domain comprises one or more alpha helices. In some embodiments, the TMB: (a) comprises or consists of 15 to 50 amino acid residues, preferably 15 to 30 amino acid residues, more preferably 18 to 25 amino acid residues; and/or (b) comprises at least 50%, at least 55%, or at least 60% of hydrophobic amino acid residues, preferably selected in the group consisting of: alanine, isoleucine, leucine, valine, phenylalanine, tryptophane and tyrosine; and/or (c) comprises at least one alpha helix.

In certain embodiments, the transmembrane domain is derived from an integral membrane protein, as further defined hereafter and in Albers et al., An “integral membrane protein” (also known as an intrinsic membrane protein) is a membrane protein that is permanently attached to the lipid membrane. In certain embodiments, the transmembrane domain is derived from an integral polytopic protein. An integral polytopic protein is one that spans the entire membrane. In certain embodiments, the transmembrane domain is derived from a single pass (trans) membrane protein, more particularly a bitopic membrane protein, e.g., of Type I or Type II. Single-pass membrane proteins cross the membrane only once (i.e., a bitopic membrane protein), while multi-pass membrane proteins weave in and out, crossing several times. Single pass transmembrane proteins can be categorized as Type I, which are positioned such that their carboxyl-terminus is towards the cytosol, or Type II, which have their amino-terminus towards the cytosol. In certain embodiments, the transmembrane domain is derived from an integral monotopic protein. An integral monotopic protein is one that is associated with the membrane from only one side and does not span the lipid bilayer completely.

In certain embodiments, the heterologous transmembrane domain is derived from a non-human sequence.

In certain embodiments, the heterologous transmembrane domain is derived from a viral sequence. The phrase “influenza”, “SARS CoV-2”, “varicella-zoster virus (VZV)”, “measles”, “rubella”, “rabies,” “Ebola,” and “smallpox” preceding the phrase “transmembrane domain sequence” indicates that the transmembrane domain sequence was derived from the virus corresponding to that name.

In certain embodiments, the heterologous transmembrane domain is derived from a viral transmembrane domain sequence selected from the group consisting of: an influenza transmembrane domain sequence, a SARS CoV-2 transmembrane domain sequence, a varicella-zoster virus (VZV) transmembrane domain sequence, a measles transmembrane domain sequence, a rubella transmembrane domain sequence, a mumps transmembrane domain sequence, a rabies transmembrane domain sequence, and an Ebola transmembrane domain sequence. These particular transmembrane domains are derived from viral sequences in viruses which have been administered to humans as vaccines (live-attenuated, inactivated or mRNA), with demonstrated strong safety profiles.

In certain embodiments, the heterologous transmembrane domain is selected from the group consisting of: an influenza hemagglutinin (HA) transmembrane domain sequence, a SARS CoV-2 spike transmembrane domain sequence, a VZV gB transmembrane domain sequence, a VZV gE transmembrane domain sequence, a VZV gI transmembrane domain sequence, a VZV gK transmembrane domain sequence, a measles F-protein transmembrane domain sequence, a rubella E1 protein transmembrane domain sequence, a rubella E2 protein transmembrane domain sequence, a mumps F-protein transmembrane domain sequence, a rabies virus glycoprotein (Rabies G) transmembrane domain sequence, and an Ebola GP protein transmembrane domain sequence.

In certain embodiments, the heterologous transmembrane domain comprises an HA transmembrane domain sequence from influenza A or influenza B, preferably from influenza A.

Exemplary viral transmembrane domain amino acid sequences of the disclosure are shown below in Table 4.

TABLE 4
Viral Transmembrane Domain (TMB) Signal
Amino Acid Sequences

| NAME | | | |
|---|---|---|---|
| OF THE | | | |
| PROTEIN | ORGANISM | STRAIN | SEQUENCE OF THE TMB |
| HA | Influenza | A/New | ILAIYSTVASSLVLLVSLGAI |
| (H1N1) | virus | Caledonia/ | SF (SEQ ID NO: 208) |
| | | 20/1999 | |
| HA | Influenza | A/Cali- | ILAIYSTVASSLVLVVSLGAI |
| (H1N1pdm) | virus | fornia/7/ | SF (SEQ ID NO: 209) |
| | | 2009 | |
| HA (H3N2) | Influenza | A/Moscow/ | ILWISFAISCFLLCVVLLGFI |
| | virus | 10/1999 | (SEQ ID NO: 241) |
| HAB | Influenza | B/Phuket/ | STAASSLAVTLMLAIFIVYMV |
| | virus | 3073/2013 | (SEQ ID NO: 242) |
| Spike | SARS | Wuhan-1 | WYIWLGFIAGLIAIVMVTIML |
| | COV-2 | | (SEQ ID NO: 243) |
| gB | VZV | Oka strain | FGALAVGLLVLAGLVAAFFAY |
| | | | (SEQ ID NO: 244) |
| gE | VZV | Oka strain | AAWTGGLAAVVLLCLVIFLIC |
| | | | (SEQ ID NO: 245) |
| gI | VZV | Oka strain | IIIPIVASVMILTAMVIVIVI |
| | | | (SEQ ID NO: 246) |
| gK | VZV | Oka strain | YFWCVQLKMIFFAWFVYGMYL |
| | | | (SEQ ID NO: 247) |
| F | Measles | Edmonston- | IVYILIAVCLGGLIGIPALIC |
| | | Zagreb | (SEQ ID NO: 248) |
| | | strain | |
| E1 | Rubella | RA27/3 | LDHAFAAFVLLVPWVLIFMVC |
| | | strain | (SEQ ID NO: 249) |
| E2 | Rubella | RA27/3 | WWQLTLGAICALLLAGLLACC |
| | | strain | (SEQ ID NO: 250) |
| F | Mumps | Miyahara | IVAALVLSILSIIISLLFCCW |
| | | strain | (SEQ ID NO: 251) |
| GP | Ebola | Mayinga- | WIPAGIGVTGVIIAVIALFCI |
| | | 76 strain | (SEQ ID NO: 252) |
| Rabies G | Rabies | Rabies | VLLSAGALTALMLIIFLMTCW |
| | | Pasteur | (SEQ ID NO: 253) |
| | | strain | |

In certain embodiments, the heterologous TMB sequence is positioned at the N-terminus or the C-terminus (e.g. C-terminus) of a polypeptide described herein.

In certain embodiments, the TMB amino acid sequence is encoded by a codon-optimized polynucleotide sequence.

In some embodiments, one or more of the C. acnes CAMP2 polypeptide, the C. acnes DsA1 polypeptide, the C. acnes DsA2 polypeptide, the C. acnes PITP polypeptide, the chimeric C. acnes DsA1/DsA2 polypeptide, the chimeric C. acnes DsA1/DsA2/PITP polypeptide and/or the chimeric C. acnes DsA1/DsA2/PITP/CAMP2 polypeptide comprise a heterologous transmembrane domain as described herein. In alternative embodiments, the C. acnes CAMP2 polypeptide, the C. acnes DsA1 polypeptide, the C. acnes DsA2 polypeptide, the C. acnes PITP polypeptide, the chimeric C. acnes DsA1/DsA2 polypeptide, the chimeric C. acnes DsA1/DsA2/PITP polypeptide and/or the chimeric C. acnes DsA1/DsA2/PITP/CAMP2 polypeptide do not comprise a heterologous transmembrane domain. Such polypeptides may comprise a secretion signal peptide sequence. In further embodiments, the polypeptides of the invention are secreted. In some embodiments, the C. acnes CAMP2 polypeptide described herein is a secreted polypeptide. In some embodiments, the C. acnes DsA1 polypeptide described herein is a secreted polypeptide. In some embodiments, the C. acnes DsA2 polypeptide described herein is a secreted polypeptide. In some embodiments, the the C. acnes PITP polypeptide described herein is a secreted polypeptide. In some embodiments, chimeric C. acnes DsA1/DsA2 polypeptide described herein is a secreted polypeptide. In some embodiments, chimeric C. acnes DsA1/DsA2/PITP polypeptide described herein is a secreted polypeptide. In some embodiments, chimeric C. acnes DsA1/DsA2/PITP/CAMP2 polypeptide described herein is a secreted polypeptide. Secreted polypeptides described herein comprise a secretion signal peptide sequence.

In certain embodiments the TMB: (a) comprises or consists of 15 to 50 amino acid residues, preferably 15 to 30 amino acid residues, more preferably 18 to 25 amino acid residues; and/or (b) comprises at least 50% of hydrophobic amino acid residues, preferably selected in the group consisting of: alanine, isoleucine, leucine, valine, phenylalanine, tryptophane and tyrosine; and/or (c) comprises at least one alpha helix.

In certain embodiments the TMB is derived from an integral membrane protein, preferably from a single pass membrane protein, more preferably from a bitopic membrane protein, even more preferably from a bitopic membrane protein of Type I.

In certain embodiments the TMB is derived from a non-human sequence.

In certain embodiments, the TMB is derived from a viral sequence.

In certain embodiments, the TMB is derived from a viral transmembrane domain sequence selected from the group consisting of: an influenza transmembrane domain sequence, and a non-influenza transmembrane domain sequence selected from the group consisting of a SARS CoV-2 transmembrane domain sequence, a varicella-zoster virus (VZV) transmembrane domain sequence, a measles transmembrane domain sequence, a rubella transmembrane domain sequence, a mumps transmembrane domain sequence, an Ebola transmembrane domain sequence, and a rabies transmembrane domain sequence.

In certain embodiments, the TMB is selected from the group consisting of: an influenza hemagglutinin (HA) transmembrane domain sequence, a SARS CoV-2 spike transmembrane domain sequence, a VZV gB transmembrane domain sequence, a VZV gE transmembrane domain sequence, a VZV gI transmembrane domain sequence, a VZV gK transmembrane domain sequence, a measles F-protein transmembrane domain sequence, a rubella E1 protein transmembrane domain sequence, a rubella E2 protein transmembrane domain sequence, a mumps F-protein transmembrane domain sequence, an Ebola GP protein transmembrane domain sequence and a rabies G protein transmembrane domain sequence, preferably wherein the TMB comprises an HA transmembrane domain sequence from influenza A or influenza B, more preferably from influenza A.

In certain embodiments, the TMB comprises an amino acid sequence selected from the group consisting of: ILAIYSTVASSLVLLVSLGAISF (SEQ ID NO: 208); ILAIYSTVASSLVLVVSLGAISF (SEQ ID NO: 209); ILWISFAISCFLLCVVLLGFI (SEQ ID NO: 241); STAASSLAVTLMLAIFIVYMV (SEQ ID NO: 242); WYIWLGFIAGLIAIVMVTIML (SEQ ID NO: 243); FGALAVGLLVLAGLVAAFFAY (SEQ ID NO: 244); AAWTGGLAAVVLLCLVIFLIC ID (SEQ NO: 245); IIIPIVASVMILTAMVIVIVI (SEQ ID NO: 246); YFWCVQLKMIFFAWFVYGMYL (SEQ ID NO: 247); IVYILIAVCLGGLIGIPALIC (SEQ ID NO: 248); LDHAFAAFVLLVPWVLIFMVC (SEQ ID NO: 249); WWQLTLGAICALLLAGLLACC (SEQ ID NO: 250); IVAALVLSILSIIISLLFCCW (SEQ ID NO: 251); WIPAGIGVTGVIIAVIALFCI (SEQ ID NO: 252); and VLLSAGALTALMLIIFLMTCW (SEQ ID NO: 253).

In certain embodiments, the TMB comprises an amino acid sequence of

(SEQ ID NO: 208)
ILAIYSTVASSLVLLVSLGAISF.

In certain embodiments, the TMB is attached to a polypeptide described herein with a linker.

In certain embodiments, the TMB is positioned at the N-terminus of a polypeptide described herein.

In certain embodiments, the TMB is positioned at the C-terminus of a polypeptide described herein.

### Linkers

In some embodiments, the polypeptides of the invention comprise linkers. In some embodiments, the polypeptides of the invention comprise two or more antigens attached via linkers.

In certain embodiments of the disclosure, the secretion signal peptide (SS) sequence or transmembrane domain (TMB) are directly fused to a polypeptide described herein (i.e., there is no linker, such as an amino acid linker, connecting the SS sequence or TMB to the polypeptide described herein).

In other embodiments, the SS sequences and TMBs of the disclosure are optionally attached to a polypeptide described herein with a linker. In certain embodiments, the linker is an amino acid linker. In the certain embodiments, the amino acid linker is 1-10 amino acids in length (e.g., the amino acid linker has a length of 1 amino acid, 2 amino acids, 3 amino acids, 4 amino acids, 5 amino acids, 6 amino acids, 7 amino acids, 8 amino acids, 9 amino acids, or 10 amino acids).

Illustrative examples of linkers include glycine polymers (Gly) n, where n is an integer of at least one, two, three, four, five, six, seven, or eight; glycine-serine polymers (GlySer) n, where n is an integer of at least one, two, three, four, five, six, seven, or eight; glycine-alanine polymers; alanine-serine polymers; and other flexible linkers known in the art.

Glycine and glycine-serine polymers are relatively unstructured and flexible, and therefore may be able to serve as a neutral tether between the SS sequence and/or TMB and the polypeptides described herein. In certain embodiments, the linker is SGS or GSG.

Other exemplary linkers include, but are not limited to, the following amino acid sequences: GGG; DGGGS (SEQ ID NO: 254); TGEKP (SEQ ID NO: 255) (Liu et al. Proc. Natl. Acad. Sci. 94:5525-5530. 1997); GGRR (SEQ ID NO: 256); (GGGGS) n (SEQ ID NO: 257), wherein n=1, 2, 3, 4 or 5 (Kim et al. Proc. Natl. Acad. Sci. 93:1156-1160. 1996); EGKSSGSGSESKVD (SEQ ID NO: 258) (Chaudhary et al. Proc. Natl. Acad. Sci. 87:1066-1070. 1990); KESGSVSSEQLAQFRSLD (SEQ ID NO: 259) (Bird et al. Science. 242:423-426. 1988), GGRRGGGS (SEQ ID NO: 260); LRQRDGERP (SEQ ID NO: 261); LRQKDGGGSERP (SEQ ID NO: 262); and GSTSGSGKPGSGEGSTKG (SEQ ID NO: 263) (Cooper et al. Blood. 101 (4): 1637-1644. 2003). Preferred linkers are shorter, e.g., consisting of 3, 4 or 5 amino acids.

Additional examples of linkers are provided in Chen et al. (Adv Drug Deliv Rev. 65 (10): 1357-1369. 2013), incorporated herein by reference.

### Compositions—Nucleic Acid and Polypeptide

The invention provides a composition comprising one or more nucleic acids of the disclosure. The invention also provides a composition comprising one or more polypeptides of the disclosure. A composition of the invention may be a pharmaceutical composition, e.g. comprising a pharmaceutically acceptable carrier, excipient or diluent. In certain embodiments, the composition of the invention is an immunogenic composition. An “immunogenic composition” means a composition comprising a nucleic acid or protein that, when administered to a subject, elicits an immune response, e.g. an antigen-specific immune response. The immune response may be a humoral (antibody) immune response or a cell-mediated immune response. The composition of the invention may be a vaccine composition. Immunogenic compositions (e.g. vaccine compositions) may elicit immunity (e.g. antibody response) against C. acnes infection. The antibody response may include antibodies that bind to the surface of C. acnes bacteria and recruit immune effector cells (e.g. effector cells of the immune system such as phagocytes). The antibodies may be capable of eliciting opsonophagocytic killing in vitro. The antibodies may be cross-reactive across a range of C. acnes strains. The antibodies may decrease or neutralise CAMP2-mediated inflammation.

“Protective immunity” or a “protective immune response”, as used herein, refers to immunity or eliciting an immune response against an infectious agent (e.g., C. acnes), which is exhibited by a subject, that prevents or ameliorates an infection or reduces at least one symptom thereof. Specifically, induction of protective immunity or a protective immune response from administration of a composition of the invention is evident by elimination or reduction of the presence of one or more symptoms of the C. acnes infection. As used herein, the term “immune response” refers to both the humoral immune response and the cell-mediated immune response. In some embodiments, treatment with a composition of the invention as described herein provides protective immunity against infection by C. acnes.

### Nucleic Acid Compositions

In one aspect, the invention provides a composition comprising a nucleic acid as described herein comprising a nucleotide sequence encoding a C. acnes CAMP2 polypeptide as described herein.

In another aspect, the invention provides a composition comprising a nucleic acid as described herein comprising a nucleotide sequence encoding a modified C. acnes CAMP2 polypeptide as described herein.

In another aspect, the invention provides a composition comprising a nucleic acid as described herein comprising a nucleotide sequence encoding a C. acnes DsA1 polypeptide as described herein.

In another aspect, the invention provides a composition comprising a nucleic acid as described herein comprising a nucleotide sequence encoding a C. acnes DsA2 polypeptide as described herein.

In another aspect, the invention provides a composition comprising a nucleic acid as described herein comprising a nucleotide sequence encoding a C. acnes PITP polypeptide as described herein.

In another aspect, the invention provides a composition comprising a nucleic acid as described herein comprising a nucleotide sequence encoding a chimeric C. acnes DsA1/DsA2 polypeptide as described herein.

In another aspect, the invention provides a composition comprising a nucleic acid as described herein comprising a nucleotide sequence encoding a chimeric C. acnes DsA1/DsA2/PITP polypeptide as described herein.

In another aspect, the invention provides a composition comprising a nucleic acid as described herein comprising a nucleotide sequence encoding a chimeric C. acnes DsA1/DsA2/PITP/CAMP2 polypeptide as described herein.

Any of the compositions described herein may comprise one or more nucleic acid encoding one or more antigens as described in WO2021/165543 (herein incorporated by reference).

### CAMP2 Compositions

In one aspect, the invention provides a composition comprising a nucleic acid as described herein comprising a nucleotide sequence encoding a C. acnes CAMP2 polypeptide as described herein.

In another aspect, the invention provides an immunogenic composition comprising a nucleic acid as described herein comprising a nucleotide sequence encoding a C. acnes CAMP2 polypeptide as described herein and wherein the nucleic acid is an mRNA.

In another aspect, the invention provides a composition comprising a nucleic acid as described herein comprising a nucleotide sequence encoding a C. acnes CAMP2 polypeptide as described herein and wherein the composition further comprises an LNP.

A composition comprising a C. acnes CAMP2 polypeptide for use in the present invention, e.g. delivered as a mRNA and/or in the form of a composition comprising a LNP, may elicit antibodies in a subject. Such antibodies may neutralise biological activity of CAMP2 polypeptide, such as CAMP2 inflammatory activity. Such antibodies may neutralise CAMP2 polypeptide co-hemolytic activity.

In some embodiments, any of the compositions described herein that comprise (a) a nucleic acid as described herein comprising a nucleotide sequence encoding a C. acnes CAMP2 polypeptide further comprise one or more of (b) (i) a nucleic acid as described herein comprising a nucleotide sequence encoding a C. acnes DsA1 polypeptide, (b) (ii) a nucleic acid as described herein comprising a nucleotide sequence encoding a C. acnes DsA2 polypeptide, (b) (iii) a nucleic acid as described herein comprising a nucleotide sequence encoding a C. acnes PITP polypeptide, (b) (iv) a nucleic acid as described herein comprising a nucleotide sequence encoding a chimeric C. acnes DsA1/DsA2 polypeptide, and (b) (v) a nucleic acid as described herein comprising a nucleotide sequence encoding a chimeric C. acnes DsA1/DsA2/PITP polypeptide. In some embodiments, the composition comprises the nucleic acid of (b) (i), the nucleic acid of (b) (ii) and the nucleic acid of (b) (iii). In some embodiments, the composition comprises the nucleic acid of (b) (v). In preferred embodiments, the composition comprises the nucleic acid of (b) (iii); and the nucleic acid of (b) (iv).

In some embodiments, any two or more nucleic acids in (a) and (b) (i)-(v) are located on the same nucleic acid molecule or on different nucleic acid molecules (e.g, wherein all the nucleic acids in the composition are on the same nucleic acid molecule or wherein all the nucleic acids in the composition are on individual nucleic acid molecules. In some embodiments, the nucleic acid in (a), the nucleic acid in (b) (iii) and the nucleic acid in (b) (iv) are located on the same nucleic acid molecule or separate nucleic acid molecules. In some embodiments, the nucleic acid in (a) and the nucleic acid in (b) (v) are located on the same nucleic acid molecule or separate nucleic acid molecules. In some embodiments, the composition comprises provides a nucleic acid that comprises a nucleotide sequence encoding a chimeric C. acnes DsA1/DsA2/PITP/CAMP2 polypeptide as described herein. Typically, the nucleic acid in (a), the nucleic acid in (b) (iii) and the nucleic acid in (b) (iv) are provided by separate nucleic acid molecules.

### Modified CAMP 2 Compositions

In another aspect, the invention provides a composition comprising a nucleic acid as described herein comprising a nucleotide sequence encoding a modified C. acnes CAMP2 polypeptide as described herein.

In some embodiments, the composition further comprises one or more of (i) a nucleic acid as described herein comprising a nucleotide sequence encoding a C. acnes DsA1 polypeptide, (ii) a nucleic acid as described herein comprising a nucleotide sequence encoding a C. acnes DsA2 polypeptide, (iii) a nucleic acid as described herein comprising a nucleotide sequence encoding a C. acnes PITP polypeptide, (iv) a nucleic acid as described herein comprising a nucleotide sequence encoding a chimeric C. acnes DsA1/DsA2 polypeptide, and (v) a nucleic acid as described herein comprising a nucleotide sequence encoding a chimeric C. acnes DsA1/DsA2/PITP polypeptide.

In some embodiments, the composition comprises the nucleic acid of (i), the nucleic acid of (ii) and the nucleic acid of (iii). In some embodiment, the composition comprises the nucleic acid of (v). In preferred embodiments, the composition comprises the nucleic acid of (iii); and the nucleic acid of (iv).

### DsA1, DsA2 and PITP Compositions

In another aspect, the invention provides a composition comprising a nucleic acid as described herein comprising one or more of (i) a nucleic acid as described herein comprising a nucleotide sequence encoding a C. acnes DsA1 polypeptide, (ii) a nucleic acid as described herein comprising a nucleotide sequence encoding a C. acnes DsA2 polypeptide, (iii) a nucleic acid as described herein comprising a nucleotide sequence encoding a C. acnes PITP polypeptide, (iv) a nucleic acid as described herein comprising a nucleotide sequence encoding a chimeric C. acnes DsA1/DsA2 polypeptide, (v) a nucleic acid as described herein comprising a nucleotide sequence encoding a chimeric C. acnes DsA1/DsA2/PITP polypeptide and (vi) a nucleic acid as described herein comprising a nucleotide sequence encoding a chimeric C. acnes DsA1/DsA2/PITP/CAMP2 polypeptide.

The combination of a C. acnes DsA1, DsA2 and PITP polypeptides may elicit an immune response against a greater number of C. acnes phylotypes compared to use of individual antigens. Using a combination of DsA1 and/or DsA2 with PITP may elicit a pool of antibodies that are cross-reactive with a greater number of C. acnes strains compared to use of individual antigens. Preferably, C. acnes DsA1 and DsA2 polypeptides are provided as a chimeric C. acnes DsA1/DsA2 polypeptide. A C. acnes PITP polypeptide may be provided as a separate polypeptide molecule, as part of a chimeric C. acnes DsA1/DsA2/PITP polypeptide or as part of a chimeric C. acnes DsA1/DsA2/PITP/CAMP2 polypeptide. Typically, a C. acnes PITP polypeptide is provided as a separate polypeptide molecule.

In some embodiments, the composition comprises the nucleic acid as described herein comprising a nucleotide sequence encoding the C. acnes DsA1 polypeptide, the nucleic acid as described herein comprising a nucleotide sequence encoding the C. acnes DsA2 polypeptide and the nucleic acid as described herein comprising a nucleotide sequence encoding the C. acnes PITP polypeptide.

In some embodiments, the composition comprises the nucleic acid as described herein comprising a nucleotide sequence encoding the chimeric C. acnes DsA1/DsA2 polypeptide as described herein and the nucleic acid as described herein comprising a nucleotide sequence encoding the C. acnes PITP polypeptide as described herein. Typically, the chimeric C. acnes DsA1/DsA2 polypeptide comprises an amino acid sequence according to SEQ ID NO: 70, or a sequence that has at least 90%, at least 91%, at least 92%, at least 93%, at least 94%, at least 95%, at least 96%, at least 97%, at least 98%, or at least 99% (e.g. at least 90% or at least 95%) identity thereto, and the C. acnes PITP polypeptide comprises the sequence according to SEQ ID NO: 73, or a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g. at least 90% or at least 95%) identity thereto. In some embodiments, the chimeric C. acnes DsA1/DsA2 polypeptide comprises the sequence of SEQ ID NO: 70 and the C. acnes PITP polypeptide comprises the sequence according to SEQ ID NO: 73. In some embodiments, the chimeric C. acnes DsA1/DsA2 polypeptide consists of an amino acid sequence according to SEQ ID NO: 70 and the C. acnes PITP polypeptide consists of an amino acid sequence according to SEQ ID NO: 73.

In some embodiments, the nucleic acid comprising a nucleotide sequence encoding the chimeric C. acnes DsA1/DsA2 polypeptide comprises a nucleotide sequence according to SEQ ID NO: 179, or a sequence that has at least 75%, at least 80%, at least 85%, at least 90%, at least 91%, at least 92%, at least 93%, at least 94%, at least 95%, at least 96%, at least 97%, at least 98%, or at least 99% (e.g. 85%) identity thereto, and the nucleic acid comprising a nucleotide sequence encoding the C. acnes PITP polypeptide comprises a nucleotide sequence according to SEQ ID NO: 182, or a sequence that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% (e.g. 75%) identity thereto.

In some embodiments, the nucleic acid comprising a nucleotide sequence encoding the chimeric C. acnes DsA1/DsA2 polypeptide consists of a nucleotide sequence encoding a secretion signal peptide sequence (e.g., a viral secretion signal peptide sequence described herein) and a sequence according to SEQ ID NO: 179, or a sequence that has at least 85% identity thereto, and the nucleic acid comprising a nucleotide sequence encoding the C. acnes PITP polypeptide consists of a nucleotide sequence encoding a secretion signal peptide sequence (e.g., a viral secretion signal peptide sequence described herein) and a sequence according to SEQ ID NO: 182, or a sequence that has at least 75% identity thereto.

Any one of the compositions of the invention may comprise a combination as described herein of the nucleic acids as described herein (e.g. they may be formulated in the same composition). The combinations as described herein of the nucleic acids as described herein may alternatively be in two or more separate compositions (e.g. as a combination of compositions for simultaneous, separate or sequential administration, (e.g. in a therapeutic or prophylactic use as described herein)).

A composition of the present disclosure comprising one or more nucleic acids of the present disclosure can also include one or more additional components such as small molecule immunopotentiators (e.g., TLR agonists). A composition of the present disclosure can also include a delivery system for a nucleic acid described herein (e.g. RNA), such as a liposome, an oil-in-water emulsion, or a microparticle. In some embodiments, the composition comprises a lipid nanoparticle (LNP). In certain embodiments, the composition comprises a nucleic acid molecule of the invention encapsulated within an LNP.

In some embodiments, a composition as described herein is in a frozen liquid form. In some embodiments, a composition as described herein is in a lyophilized form (e.g. in a lyophilized form).

### Polypeptide Compositions

In one aspect, the invention provides a composition comprising a C. acnes CAMP2 polypeptide as described herein.

In another aspect, the invention provides a composition comprising a modified C. acnes CAMP2 polypeptide as described herein.

In another aspect, the invention provides a composition comprising a C. acnes DsA1 polypeptide as described herein.

In another aspect, the invention provides a composition comprising a C. acnes DsA2 polypeptide as described herein.

In another aspect, the invention provides a composition comprising a C. acnes PITP polypeptide as described herein.

In another aspect, the invention provides a composition comprising a chimeric C. acnes DsA1/DsA2 polypeptide as described herein.

In another aspect, the invention provides a composition comprising a chimeric C. acnes DsA1/DsA2/PITP polypeptide as described herein.

In another aspect, the invention provides a composition comprising a chimeric C. acnes DsA1/DsA2/PITP/CAMP2 polypeptide as described herein.

Any of the compositions described herein may comprise any of the polypeptide antigens as described in WO2021/165543.

### CAMP 2 Compositions

In one aspect, the invention provides a composition comprising a C. acnes CAMP2 polypeptide as described herein.

In some embodiments, the composition comprising (a) a C. acnes CAMP2 polypeptide as described herein further comprises one or more of (b) (i) a C. acnes DsA1 polypeptide as described herein, (b) (ii) a C. acnes DsA2 polypeptide as described herein, (b) (iii) a C. acnes PITP polypeptide as described herein, (b) (iv) a chimeric C. acnes DsA1/DsA2 polypeptide as described herein, and (b) (v) a chimeric C. acnes DsA1/DsA2/PITP polypeptide as described herein. In some embodiments, the composition comprises the polypeptide of (b) (i), the polypeptide of (b) (ii) and the polypeptide of (b) (iii). In some embodiments, the composition comprises the polypeptide of (b) (v). In preferred embodiments, the composition comprises the polypeptide of (b) (iii); and the polypeptide of (b) (iv).

In some embodiments, the polypeptides of (a), (b) (iii) and (b) (iv) are provided as a chimeric polypeptide, e.g. a chimeric C. acnes DsA1/DsA2/PITP/CAMP2 polypeptide as described herein. In some embodiments, the polypeptides of (a) and (b) (v) are provided as a chimeric polypeptide, e.g. a chimeric C. acnes DsA1/DsA2/PITP/CAMP2 polypeptide as described herein. Typically, the polypeptides of (a), (b) (iii) and (b) (iv) are provided as separate polypeptides.

### Modified CAMP 2 Compositions

In another aspect, the invention provides a composition comprising a modified C. acnes CAMP2 polypeptide as described herein.

In some embodiments, the composition comprising a modified C. acnes CAMP2 polypeptide as described herein further comprises one or more of (i) a C. acnes DsA1 polypeptide as described herein, (ii) a C. acnes DsA2 polypeptide as described herein, (iii) a C. acnes PITP polypeptide as described herein, (iv) a chimeric C. acnes DsA1/DsA2 polypeptide as described herein, and (v) a chimeric C. acnes DsA1/DsA2/PITP polypeptide as described herein.

In some embodiments, the composition comprises the polypeptide of (i), the polypeptide of (ii) and the polypeptide of (iii). In some embodiments, the composition comprises the polypeptide of (v). In preferred embodiments, the composition comprises the polypeptide of (iii); and the polypeptide of (iv).

### DsA1, DsA2 and PITP Compositions

In another aspect, the invention provides a composition comprising one or more of (i) a C. acnes DsA1 polypeptide as described herein, (ii) a C. acnes DsA2 polypeptide as described herein, (iii) a C. acnes PITP polypeptide as described herein, (iv) a chimeric C. acnes DsA1/DsA2 polypeptide as described herein, (v) a chimeric C. acnes DsA1/DsA2/PITP polypeptide as described herein, and (vi) a chimeric DsA1/DsA2/PITP/CAMP2 polypeptide as described herein.

In some embodiments, the composition comprises the C. acnes DsA1 polypeptide as described herein, the C. acnes DsA2 polypeptide as described herein, and the C. acnes PITP polypeptide as described herein.

In some embodiments, the composition comprises the chimeric C. acnes DsA1/DsA2 polypeptide as described herein and the C. acnes PITP polypeptide as described herein. Typically, the chimeric C. acnes DsA1/DsA2 polypeptide comprises an amino acid sequence according to SEQ ID NO: 70, or a sequence that has at least 90%, at least 91%, at least 92%, at least 93%, at least 94%, at least 95%, at least 96%, at least 97%, at least 98%, or at least 99% (e.g. at least 90% or at least 95%) identity thereto, and the C. acnes PITP polypeptide comprises the sequence according to SEQ ID NO: 73, or a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g. at least 90% or at least 95%) identity thereto. In some embodiments, the chimeric C. acnes DsA1/DsA2 polypeptide comprises the sequence of SEQ ID NO: 70 and the C. acnes PITP polypeptide comprises the sequence according to SEQ ID NO: 73. In some embodiments, the chimeric C. acnes DsA1/DsA2 polypeptide consists of an amino acid sequence according to SEQ ID NO: 70 and the C. acnes PITP polypeptide consists of an amino acid sequence according to SEQ ID NO: 73.

Any one of the compositions of the invention may comprise a combination as described herein of the polypeptides as described herein (e.g. they may be formulated in the same composition). The combinations as described herein of the polypeptides as described herein may alternatively be in two or more separate compositions (e.g. as a combination of compositions for simultaneous, separate or sequential administration, (e.g. in a therapeutic or prophylactic use as described herein)).

A composition of the present disclosure comprising one or more polypeptides of the present disclosure may comprise an adjuvant as described herein. As used herein, an “adjuvant” refers to a substance or vehicle that enhances the immune response to an antigen. Adjuvants can include, without limitation, a suspension of minerals (e.g., alum, aluminum hydroxide, or phosphate) on which antigen is adsorbed; a water-in-oil or oil-in-water emulsion in which antigen solution is emulsified in mineral oil or in water (e.g., Freund's incomplete adjuvant). Sometimes killed mycobacteria is included (e.g., Freund's complete adjuvant) to further enhance antigenicity. Adjuvants can include squalene based oil in water emulsion adjuvants (e.g. AF03 e.g as described in WO2007006939 and U.S. Pat. No. 8,703,095; AS03, e.g. as described in WO1995017209, WO1995017210 and U.S. Pat. Nos. 6,623,739, 7,029,678 and 7,510,698; and MF59, e.g as described in WO1990014837 and U.S. Pat. Nos. 6,299,884 and 6,451,325). Immuno-stimulatory oligonucleotides (e.g., a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants can also include biological molecules, such as Toll-Like Receptor (TLR) agonists (e.g. AS01, e.g. as described in WO2007068907 and U.S. Pat. Nos. 10,039,823 and 10,143,745; SPA14, e.g. as described in WO2022090359; and LEQ, e.g. as described in WO2023056089) and costimulatory molecules.

In some embodiments, the adjuvant is selected from the group consisting of: Aluminum based adjuvant (e.g. AlOOH), Squalene based oil in water emulsion adjuvants (e.g. AF03, AS03, MF59) and Liposome-based adjuvants comprising a saponin and a TLR4 agonist (e.g. SPA14, LEQ, AS01).

In preferred embodiments, the adjuvant is selected from the group consisting of AlOOH, AF03 and SPA14.

In some embodiments, a composition as described herein is in a frozen liquid form. In some embodiments, a composition as described herein is in a lyophilized form.

### Combinations—Nucleic Acid and Polypeptide

### Nucleic Acid Combinations

The invention provides combinations comprising two or more nucleic acids of the invention.

### CAMP 2 Combinations

In some aspects, the invention provides a combination comprising a nucleic acid comprising a nucleotide sequence encoding a C. acnes CAMP2 polypeptide; and one or more of: (i) a nucleic acid comprising a nucleotide sequence encoding a C. acnes DsA1 polypeptide; (ii) a nucleic acid comprising a nucleotide sequence encoding a C. acnes DsA2 polypeptide; (iii) a nucleic acid comprising a nucleotide sequence encoding a C. acnes PITP polypeptide; (iv) a nucleic acid comprising a nucleotide sequence encoding a chimeric C. acnes DsA1/DsA2 polypeptide; and (v) a nucleic acid comprising a nucleotide sequence encoding a chimeric C. acnes DsA1/DsA2/PITP polypeptide.

In some embodiments, the combination comprises the nucleic acid of (i), the nucleic acid of (ii) and the nucleic acid of (iii). In some embodiments, the composition comprises the nucleic acid of (v). In preferred embodiments, the composition comprises the nucleic acid of (iii); and the nucleic acid of (iv). In some embodiments, the combination comprises the nucleic acid of (v). In preferred embodiments, the combination comprises the nucleic acid of (iii), and the nucleic acid of (iv).

In some embodiments, the combination comprises a nucleic acid comprising a nucleotide sequence encoding a C. acnes CAMP2 polypeptide, the nucleic acid in (iii) and the nucleic acid in (iv). In some embodiments, the combination comprises a nucleic acid comprising a nucleotide sequence encoding a C. acnes CAMP2 polypeptide and the nucleic acid in (v). In some embodiments, such combinations are provided as a nucleic acid that comprises a nucleotide sequence encoding a chimeric C. acnes DsA1/DsA2/PITP/CAMP2 polypeptide as described herein. Typically, the nucleic acid comprising a nucleotide sequence encoding a C. acnes CAMP2 polypeptide, the nucleic acid in (iii) and the nucleic acid in (iv) are provided by separate nucleic acid molecules.

### Modified CAMP 2 Combinations

In another aspect, the invention provides a combination comprising a nucleic acid as described herein comprising a nucleotide sequence encoding a modified C. acnes CAMP2 polypeptide as described herein and one or more of (i) a nucleic acid as described herein comprising a nucleotide sequence encoding a C. acnes DsA1 polypeptide, (ii) a nucleic acid as described herein comprising a nucleotide sequence encoding a C. acnes DsA2 polypeptide, (iii) a nucleic acid as described herein comprising a nucleotide sequence encoding a C. acnes PITP polypeptide, (iv) a nucleic acid as described herein comprising a nucleotide sequence encoding a chimeric C. acnes DsA1/DsA2 polypeptide, and (v) a nucleic acid as described herein comprising a nucleotide sequence encoding a chimeric C. acnes DsA1/DsA2/PITP polypeptide.

In some embodiments, the combination comprises the nucleic acid of (i), the nucleic acid of (ii) and the nucleic acid of (iii). In some embodiment, the combination comprises the nucleic acid of (v). In preferred embodiments, the combination comprises the nucleic acid of (iii); and the nucleic acid of (iv).

### DsA1, DsA2 and PITP Combinations

In one aspect, the invention provides a combination comprising one or more of: (i) a nucleic acid comprising a nucleotide sequence encoding a C. acnes DsA1 polypeptide; (ii) a nucleic acid comprising a nucleotide sequence encoding a C. acnes DsA2 polypeptide; (iii) a nucleic acid comprising a nucleotide sequence encoding a C. acnes PITP polypeptide; (iv) a nucleic acid comprising a nucleotide sequence encoding a chimeric C. acnes DsA1/DsA2 polypeptide; and (v) a nucleic acid comprising a nucleotide sequence encoding a chimeric C. acnes DsA1/DsA2/PITP polypeptide.

In some embodiments, the combination comprises the nucleic acid as described herein comprising a nucleotide sequence encoding the C. acnes DsA1 polypeptide, the nucleic acid as described herein comprising a nucleotide sequence encoding the C. acnes DsA2 polypeptide and the nucleic acid as described herein comprising a nucleotide sequence encoding the C. acnes PITP polypeptide.

In some embodiments, the combination comprises the nucleic acid as described herein comprising a nucleotide sequence encoding the chimeric C. acnes DsA1/DsA2 polypeptide as described herein and the nucleic acid as described herein comprising a nucleotide sequence encoding the C. acnes PITP polypeptide as described herein. Typically, the chimeric C. acnes DsA1/DsA2 polypeptide comprises an amino acid sequence according to SEQ ID NO: 70, or a sequence that has at least 90%, at least 91%, at least 92%, at least 93%, at least 94%, at least 95%, at least 96%, at least 97%, at least 98%, or at least 99% (e.g. at least 90% or at least 95%) identity thereto, and the C. acnes PITP polypeptide comprises the sequence according to SEQ ID NO: 73, or a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g. at least 90% or at least 95%) identity thereto. In some embodiments, the chimeric C. acnes DsA1/DsA2 polypeptide comprises the sequence of SEQ ID NO: 70 and the C. acnes PITP polypeptide comprises the sequence according to SEQ ID NO: 73. In some embodiments, the chimeric C. acnes DsA1/DsA2 polypeptide consists of an amino acid sequence according to SEQ ID NO: 70 and the C. acnes PITP polypeptide consists of an amino acid sequence according to SEQ ID NO: 73.

In some embodiments, the nucleic acid comprising a nucleotide sequence encoding the chimeric C. acnes DsA1/DsA2 polypeptide comprises a nucleotide sequence according to SEQ ID NO: 179 or a sequence that has at least 75%, at least 80%, at least 85%, at least 90%, at least 91%, at least 92%, at least 93%, at least 94%, at least 95%, at least 96%, at least 97%, at least 98%, or at least 99% (e.g. 85%) identity thereto, and the nucleic acid comprising a nucleotide sequence encoding the C. acnes PITP polypeptide comprises a nucleotide sequence according to any one of SEQ ID NO: 182 or a sequence that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% (e.g. 75%) identity thereto.

In some embodiments, the nucleic acid comprising a nucleotide sequence encoding the chimeric C. acnes DsA1/DsA2 polypeptide consists of a nucleotide sequence encoding a secretion signal peptide sequence (e.g., a viral secretion signal peptide sequence described herein) and a sequence according to SEQ ID NO: 179 or a sequence that has at least 85% identity thereto, and the nucleic acid comprising a nucleotide sequence encoding the C. acnes PITP polypeptide consists of a nucleotide sequence encoding a secretion signal peptide sequence (e.g., a viral secretion signal peptide sequence described herein) and a sequence according to SEQ ID NO: 182, or a sequence that has at least 75% identity thereto.

### Polypeptide Combinations

The invention provides combinations comprising two or more polypeptides of the invention.

### CAMP 2 Combinations

In some aspects, the invention provides a combination comprising a C. acnes CAMP2 polypeptide; and one or more of: (i) a C. acnes DsA1 polypeptide as described herein, (ii) a C. acnes DsA2 polypeptide as described herein, (iii) a C. acnes PITP polypeptide as described herein, (iv) a chimeric C. acnes DsA1/DsA2 polypeptide as described herein, and (v) a chimeric C. acnes DsA1/DsA2/PITP polypeptide as described herein.

In some embodiments, the combination comprises the polypeptide of (i), the polypeptide of (ii) and the polypeptide of (iii). In some embodiments, the combination comprises the polypeptide of (v). In preferred embodiments, the combination comprises the polypeptide of (iii); and the polypeptide of (iv). In some embodiments, such combinations are provided as a chimeric polypeptide, e.g. a chimeric C. acnes DsA1/DsA2/PITP/CAMP2 polypeptide as described herein. Typically, the C. acnes CAMP2 polypeptide, the polypeptide of (iii) and the polypeptide of (iv) are provided as separate polypeptides.

### Modified CAMP 2 Combinations

In another aspect, the invention provides a combination comprising a modified C. acnes CAMP2 polypeptide as described herein and one or more of (i) a C. acnes DsA1 polypeptide as described herein, (ii) a C. acnes DsA2 polypeptide as described herein, (iii) a C. acnes PITP polypeptide as described herein, (iv) a chimeric C. acnes DsA1/DsA2 polypeptide as described herein, and (v) a chimeric C. acnes DsA1/DsA2/PITP polypeptide as described herein.

In some embodiments, the combination comprises the polypeptide of (i), the polypeptide of (ii) and the polypeptide of (iii). In some embodiments, the combination comprises the polypeptide of (v). In preferred embodiments, the combination comprises the polypeptide of (iii); and the polypeptide of (iv).

### DsA1, DsA2 and PITP Compositions

In one aspect, the invention provides a combination comprising one or more of (i) a C. acnes DsA1 polypeptide as described herein, (ii) a C. acnes DsA2 polypeptide as described herein, (iii) a C. acnes PITP polypeptide as described herein, (iv) a chimeric C. acnes DsA1/DsA2 polypeptide as described herein, and (v) a chimeric C. acnes DsA1/DsA2/PITP polypeptide as described herein.

In some embodiments, the combination comprises the C. acnes DsA1 polypeptide as described herein, the C. acnes DsA2 polypeptide as described herein, and the C. acnes PITP polypeptide as described herein.

In some embodiments, the composition comprises the chimeric C. acnes DsA1/DsA2 polypeptide as described herein and the C. acnes PITP polypeptide as described herein. Typically, the chimeric C. acnes DsA1/DsA2 polypeptide comprises an amino acid sequence according to SEQ ID NO: 70, or a sequence that has at least 90%, at least 91%, at least 92%, at least 93%, at least 94%, at least 95%, at least 96%, at least 97%, at least 98%, or at least 99% (e.g. at least 90% or at least 95%) identity thereto, and the C. acnes PITP polypeptide comprises the sequence according to SEQ ID NO: 73, or a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g. at least 90% or at least 95%) identity thereto. In some embodiments, the chimeric C. acnes DsA1/DsA2 polypeptide comprises the sequence of SEQ ID NO: 70 and the C. acnes PITP polypeptide comprises the sequence according to SEQ ID NO: 73. In some embodiments, the chimeric C. acnes DsA1/DsA2 polypeptide consists of an amino acid sequence according to SEQ ID NO: 70 and the C. acnes PITP polypeptide consists of an amino acid sequence according to SEQ ID NO: 73.

### LNPs

In certain embodiments, the composition of the invention (e.g. the composition comprising a nucleic acid of the invention) further comprises a lipid nanoparticle (LNP). In certain embodiments, the nucleic acid of the invention is encapsulated in the LNP.

The LNPs of the disclosure may comprise four categories of lipids: (i) an ionizable lipid (e.g., a cationic lipid); (ii) a PEGylated lipid; (iii) a cholesterol-based lipid, and (iv) a helper lipid.

### A. Ionizable Lipids

An ionizable lipid facilitates mRNA encapsulation and may be a cationic lipid. A cationic lipid affords a positively charged environment at low pH to facilitate efficient encapsulation of the negatively charged mRNA drug substance.

In some embodiments, the cationic lipid is OF-02:

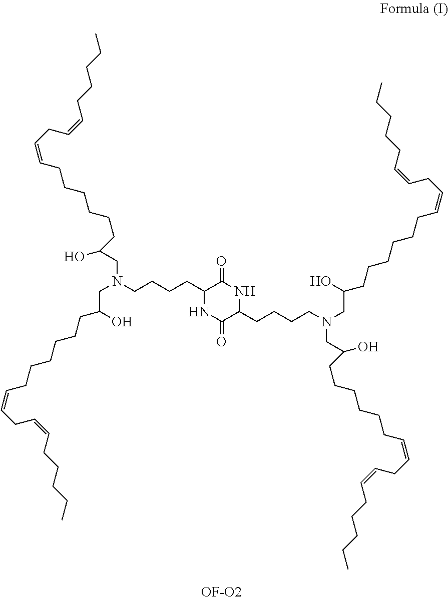 [SMILES: CCCCC/C=C\C/C=C\CCCCCCC(O)CN(CCCCC1NC(=O)C(CCCCN(CC(O)CCCCCC/C=C\C/C=C\CCCCC)CC(O)CCCCCC/C=C\C/C=C\CCCCC)NC1=O)CC(O)CCCCCC/C=C\C/C=C\CCCCC ; "OF-O2"]

OF-02 is a non-degradable structural analog of OF-Deg-Lin. OF-Deg-Lin contains degradable ester linkages to attach the diketopiperazine core and the doubly-unsaturated tails, whereas OF-02 contains non-degradable 1,2-amino-alcohol linkages to attach the same diketopiperazine core and the doubly-unsaturated tails (Fenton et al., Adv Mater. (2016) 28:2939; U.S. Pat. No. 10,201,618). An exemplary LNP formulation herein, Lipid A, contains OF-2.

In some embodiments, the cationic lipid is cKK-E10 (Dong et al., PNAS (2014) 111 (11): 3955-60; U.S. Pat. No. 9,512,073):

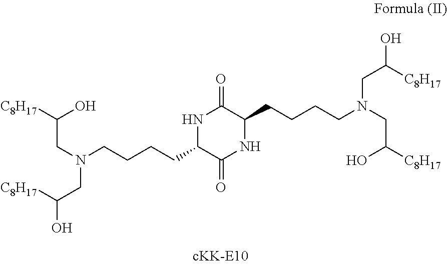

An exemplary LNP formulation herein, Lipid B, contains cKK-E10.

In some embodiments, the cationic lipid is GL-HEPES-E3-E10-DS-3-E18-1 (2-(4-(2-((3-(Bis((Z)-2-hydroxyoctadec-9-en-1-yl)amino) propyl)disulfaneyl)ethyl) piperazin-1-yl)ethyl 4-(bis(2-hydroxydecyl)amino)butanoate), which is a HEPES-based disulfide cationic lipid with a piperazine core, having the Formula III:

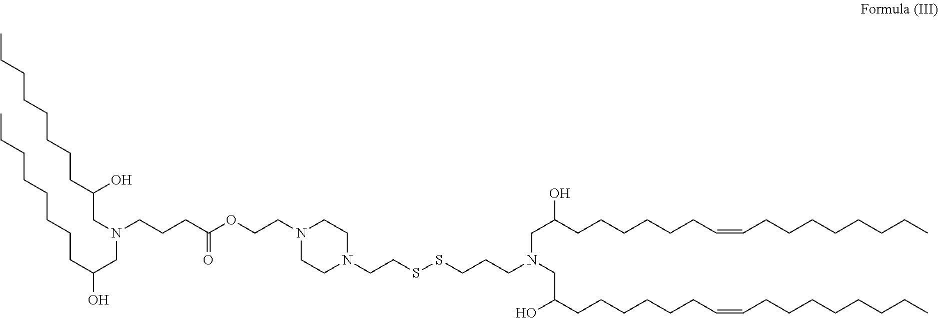 [SMILES: CCCCCCCC/C=C\CCCCCCC(O)CN(CCCSSCCN1CCN(CCOC(=O)CCCN(CC(O)CCCCCCCC)CC(O)CCCCCCCC)CC1)CC(O)CCCCCC/C=C\CCCCCCCC]

An exemplary LNP formulation herein, Lipid C, contains GL-HEPES-E3-E10-DS-3-E18-1. Lipid C has the same composition as Lipid A or Lipid B but for the difference in the cationic lipid.

Typically, the cationic lipid is GL-HEPES-E3-E12-DS-4-E10 (2-(4-(2-((3-(bis(2-hydroxydecyl)amino)butyl)disulfaneyl)ethyl) piperazin-1-yl)ethyl 4-(bis(2-hydroxydodecyl)amino)butanoate), which is a HEPES-based disulfide cationic lipid with a piperazine core, having the Formula IV:

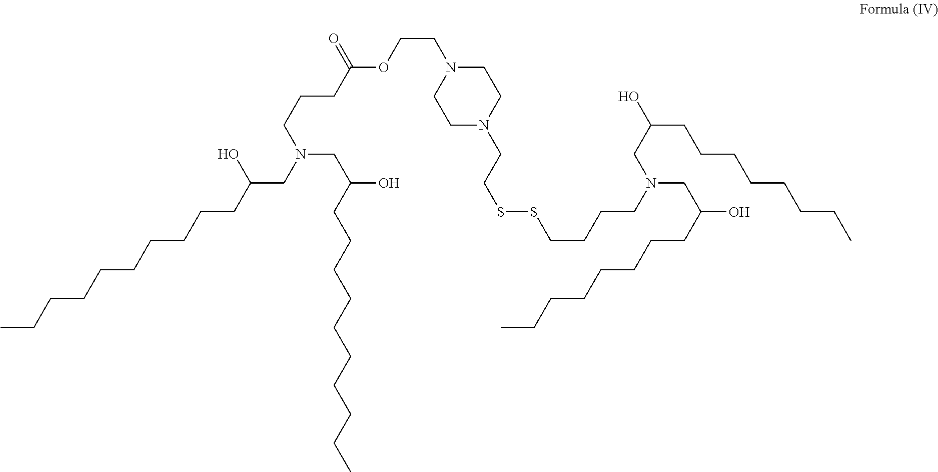 [SMILES: CCCCCCCCCCC(O)CN(CCCC(=O)OCCN1CCN(CCSSCCCCN(CC(O)CCCCCCCC)CC(O)CCCCCCCC)CC1)CC(O)CCCCCCCCCC]

An exemplary LNP formulation herein, Lipid D, contains GL-HEPES-E3-E12-DS-4-E10. Lipid D has the same composition as Lipid A or Lipid B but for the difference in the cationic lipid.

In some embodiments, the cationic lipid is GL-HEPES-E3-E12-DS-3-E14 (2-(4-(2-((3-(Bis(2-hydroxytetradecyl)amino) propyl)disulfaneyl)ethyl) piperazin-1-yl)ethyl 4-(bis(2-hydroxydodecyl)amino)butanoate), which is a HEPES-based disulfide cationic lipid with a piperazine core, having the Formula V:

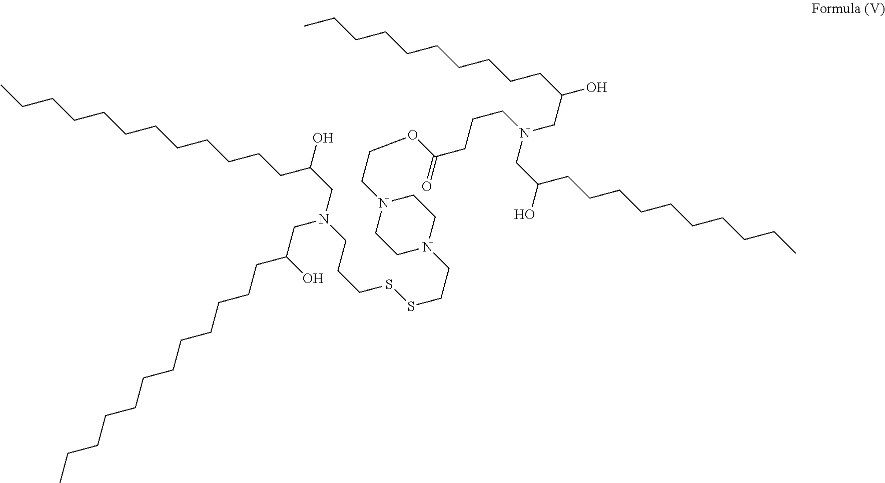 [SMILES: CCCCCCCCCCCCC(O)CN(CCCSSCCN1CCN(CCOC(=O)CCCN(CC(O)CCCCCCCCCC)CC(O)CCCCCCCCCC)CC1)CC(O)CCCCCCCCCCCC]

An exemplary LNP formulation herein, Lipid E, contains GL-HEPES-E3-E12-DS-3-E14. Lipid E has the same composition as Lipid A or Lipid B but for the difference in the cationic lipid.

The cationic lipids GL-HEPES-E3-E10-DS-3-E18-1 (III), GL-HEPES-E3-E12-DS-4-E10 (IV), and GL-HEPES-E3-E12-DS-3-E14 (V) can be synthesized according to the general procedure set out in Scheme 1:

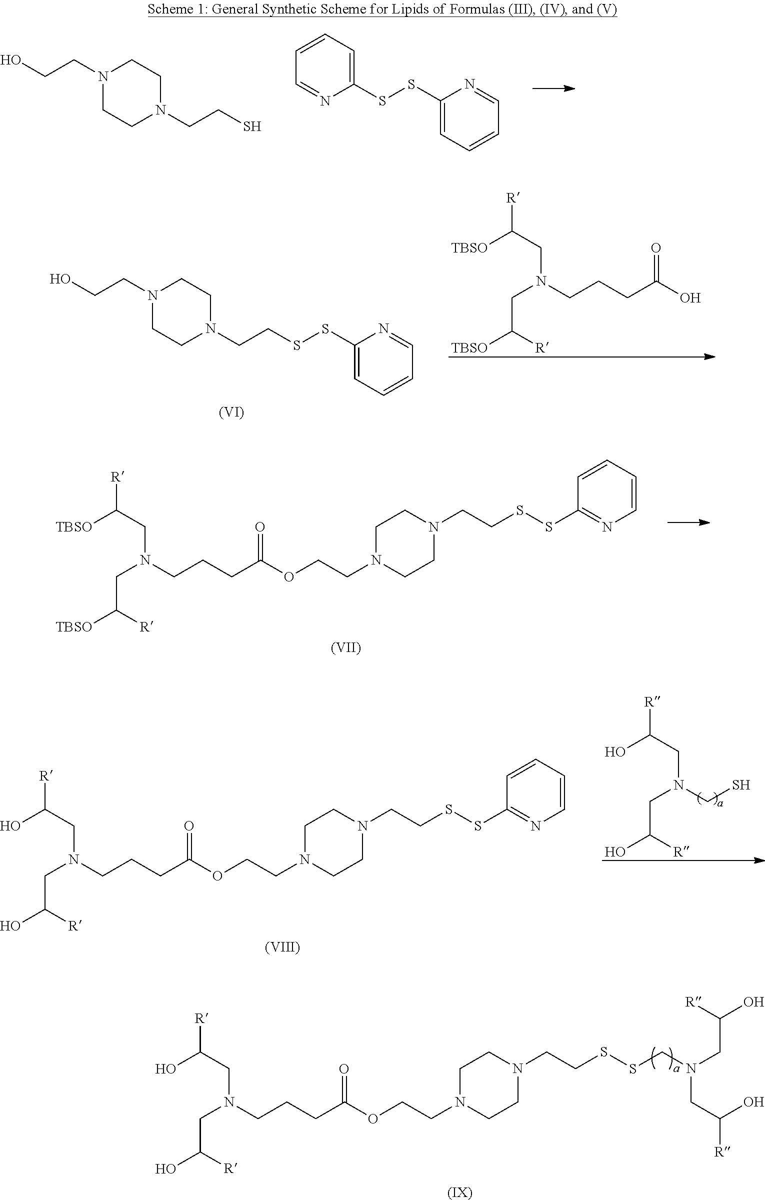

In some embodiments, the cationic lipid is MC3, having the Formula VI:

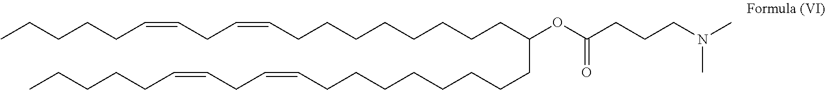 [SMILES: CCCCC/C=C\C/C=C\CCCCCCCCC(CCCCCCCC/C=C\C/C=C\CCCCC)OC(=O)CCCN(C)C]

In some embodiments, the cationic lipid is SM-102 (9-heptadecanyl 8-{(2-hydroxyethyl) [6-oxo-6-(undecyloxy) hexyl]amino}octanoate), having the Formula VII:

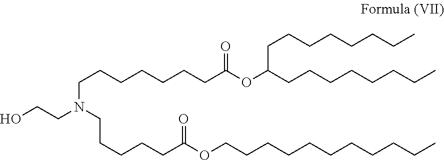 [SMILES: CCCCCCCCCCCOC(=O)CCCCCN(CCO)CCCCCCCC(=O)OC(CCCCCCCC)CCCCCCCC]

In some embodiments, the cationic lipid is ALC-0315 [(4-hydroxybutyl) azanediyl]di(hexane-6,1-diyl)bis(2-hexyldecanoate), having the Formula VIII:

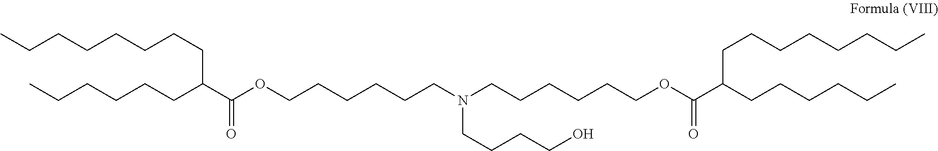 [SMILES: CCCCCCCCC(CCCCCC)C(=O)OCCCCCCN(CCCCO)CCCCCCOC(=O)C(CCCCCC)CCCCCCCC]

In some embodiments, the cationic lipid is cOrn-EE1, having the Formula IX:

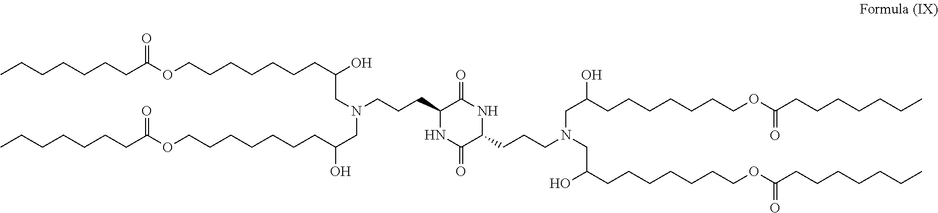 [SMILES: CCCCCCCC(=O)OCCCCCCCC(O)CN(CCC[C@@H]1NC(=O)[C@@H](CCCN(CC(O)CCCCCCCOC(=O)CCCCCCC)CC(O)CCCCCCCOC(=O)CCCCCCC)NC1=O)CC(O)CCCCCCCOC(=O)CCCCCCC]

In some embodiments, the cationic lipid may be selected from the group comprising cKK-E10; OF-02; [(6Z,9Z,28Z,31Z)-heptatriaconta-6,9,28,31-tetraen-19-yl]4-(dimethylamino)butanoate (D-Lin-MC3-DMA); 2,2-dilinoleyl-4-dimethylaminoethyl-[1,3]-dioxolane (dLin-KC2-DMA); 1,2-dilinoleyloxy-N,N-dimethyl-3-aminopropane (dLin-DMA); di((Z)-non-2-en-1-yl) 9-((4-(dimethylamino)butanoyl)oxy) heptadecanedioate (L319); 9-heptadecanyl 8-{(2-hydroxyethyl) [6-oxo-6-(undecyloxy) hexyl]amino}octanoate (SM-102); [(4-hydroxybutyl) azanediyl]di(hexane-6,1-diyl)bis(2-hexyldecanoate) (ALC-0315); [3-(dimethylamino)-2-[(Z)-octadec-9-enoyl]oxypropyl] (Z)-octadec-9-enoate (DODAP); 2,5-bis(3-aminopropylamino)-N-[2-[di(heptadecyl)amino]-2-oxoethyl]pentanamide (DOGS); [(3S,8S,9S,10R,13R,14S,17R)-10,13-dimethyl-17-[(2R)-6-methylheptan-2-yl]-2,3,4,7,8,9,11,12,14, 15, 16, 17-dodecahydro-1H-cyclopenta[a]phenanthren-3-yl] N-[2-(dimethylamino)ethyl]carbamate (DC-Chol); tetrakis(8-methylnonyl) 3,3′,3″,3′″-(((methylazanediyl)bis(propane-3,1 diyl))bis(azanetriyl))tetrapropionate (306Oi10); decyl(2-(dioctylammonio)ethyl)phosphate (9A1P9); ethyl 5,5-di((Z)-heptadec-8-en-1-yl)-1-(3-(pyrrolidin-1-yl) propyl)-2,5-dihydro-1H-imidazole-2-carboxylate (A2-Iso5-2DC18); bis(2-(dodecyldisulfanyl)ethyl) 3,3′-((3-methyl-9-oxo-10-oxa-13,14-dithia-3,6-diazahexacosyl) azanediyl)dipropionate (BAME-O16B); 1,1′-((2-(4-(2-((2-(bis(2-hydroxydodecyl)amino)ethyl) (2-hydroxydodecyl)amino)ethyl) piperazin-1-yl)ethyl) azanediyl)bis(dodecan-2-ol) (C12-200); 3,6-bis(4-(bis(2-hydroxydodecyl)amino)butyl) piperazine-2,5-dione (cKK-E12); hexa (octan-3-yl) 9,9′,9″,9″′,9″″,9′″″-((((benzene-1,3,5-tricarbonyl)tris(azanediyl)) tris(propane-3,1-diyl))tris(azanetriyl)) hexanonanoate (FTT5); (((3,6-dioxopiperazine-2,5-diyl)bis(butane-4,1-diyl))bis(azanetriyl)) tetrakis(ethane-2,1-diyl) (9Z,9′Z,9″Z,9′″Z,12Z,12′Z,12″Z,12′″Z)-tetrakis(octadeca-9,12-dienoate) (OF-Deg-Lin); TT3; N1,N3,N5-tris(3-(didodecylamino) propyl)benzene-1,3,5-tricarboxamide; N1-[2-((1S)-1-[(3-aminopropyl)amino]-4-[di(3-aminopropyl)amino]butylcarboxamido)ethyl]-3,4-di[oleyloxy]-benzamide (MVL5); heptadecan-9-yl 8-((2-hydroxyethyl) (8-(nonyloxy)-8-oxooctyl)amino) octanoate (Lipid 5); GL-HEPES-E3-E10-DS-3-E18-1; GL-HEPES-E3-E12-DS-4-E10; GL-HEPES-E3-E12-DS-3-E14; and combinations thereof.

In some embodiments, the cationic lipid is IM-001, having the Formula X (EP23306049.0):

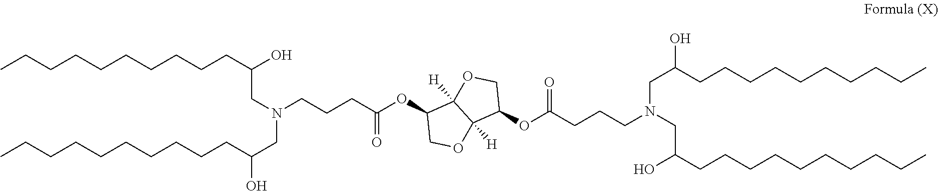 [SMILES: CCCCCCCCCCC(O)CN(CCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCCCCCC]

An exemplary LNP formulation herein, Lipid F, contains IM-001. Lipid F has the same composition as Lipid A or Lipid B but for the difference in the cationic lipid.

The cationic lipid IM-001 (X) can be synthesized according to the general procedure set out in Scheme 2:

### Scheme 2: General Synthetic Scheme for Lipid of Formula (X)

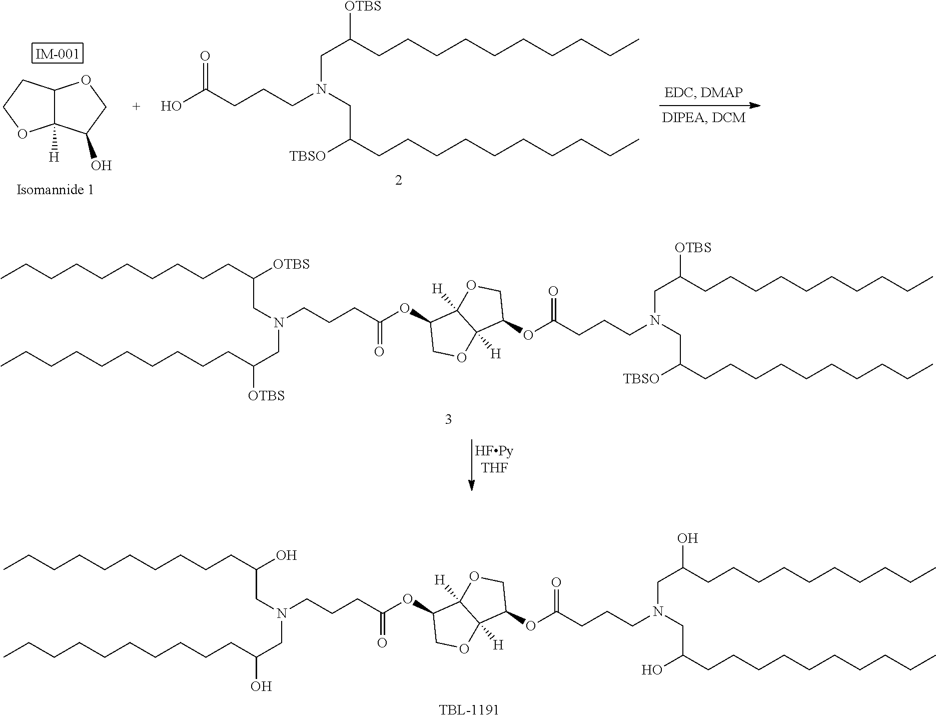

Scheme 2 may be performed as described in Example 12.

In some embodiments, the cationic lipid is IS-001, having the Formula XI (EP23306049.0):

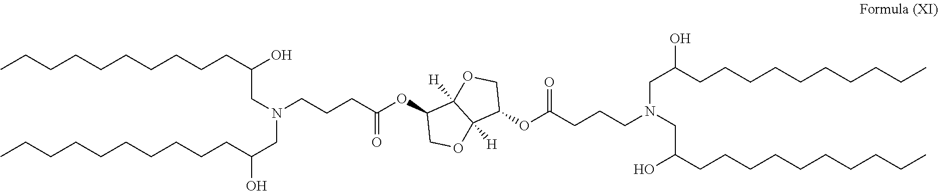 [SMILES: CCCCCCCCCCC(O)CN(CCCC(=O)O[C@H]1CO[C@H]2[C@@H]1OC[C@H]2OC(=O)CCCN(CC(O)CCCCCCCCCC)CC(O)CCCCCCCCCC)CC(O)CCCCCCCCCC]

An exemplary LNP formulation herein, Lipid G, contains IS-001. Lipid G has the same composition as Lipid A or Lipid B but for the difference in the cationic lipid.

The cationic lipid IS-001 (XI) can be synthesized according to the general procedure set out in Scheme 3:

### Scheme 3: General Synthetic Scheme for Lipid of Formula (XI)

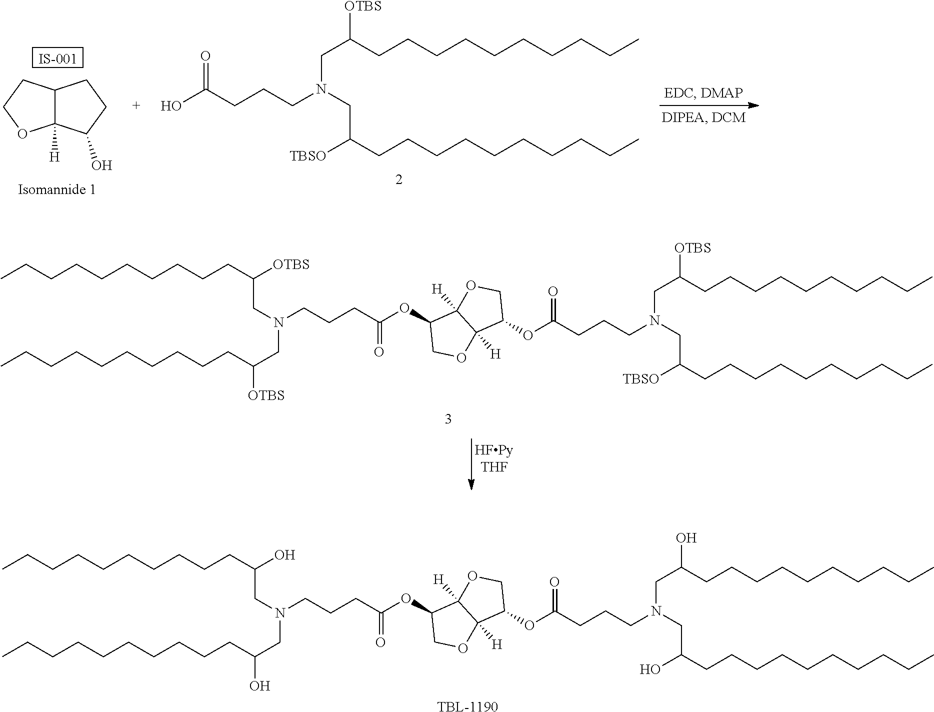

Scheme 3 may be performed as described in Example 14.

In some embodiments, the cationic lipid is biodegradable.

In some embodiments, the cationic lipid is not biodegradable.

In some embodiments, the cationic lipid is cleavable.

In some embodiments, the cationic lipid is not cleavable.

Cationic lipids are described in further detail in Dong et al. (PNAS. 111 (11): 3955-60. 2014); Fenton et al. (Adv Mater. 28:2939. 2016); U.S. Pat. Nos. 9,512,073; and 10,201,618, each of which is incorporated herein by reference.

### B. PEGylated Lipids

The PEGylated lipid component provides control over particle size and stability of the nanoparticle. The addition of such components may prevent complex aggregation and provide a means for increasing circulation lifetime and increasing the delivery of the lipid-nucleic acid pharmaceutical composition to target tissues (Klibanov et al. FEBS Letters 268 (1): 235-7. 1990). These components may be selected to rapidly exchange out of the pharmaceutical composition in vivo (see, e.g., U.S. Pat. No. 5,885,613).

Contemplated PEGylated lipids include, but are not limited to, a polyethylene glycol (PEG) chain of up to 5 kDa in length covalently attached to a lipid with alkyl chain(s) of C₆-C₂₀ (e.g., C₈, C₁₀, C₁₂, C₁₄, C₁₆, or C₁₈) length, such as a derivatized ceramide (e.g., N-octanoyl-sphingosine-1-[succinyl(methoxypolyethylene glycol)] (C8 PEG ceramide)). In some embodiments, the PEGylated lipid is 1,2-dimyristoyl-rac-glycero-3-methoxypolyethylene glycol (DMG-PEG); 1,2-distearoyl-sn-glycero-3-phosphoethanolamine-polyethylene glycol (DSPE-PEG); 1,2-dilauroyl-sn-glycero-3-phosphoethanolamine-polyethylene glycol (DLPE-PEG); or 1,2-distearoyl-rac-glycero-polyethelene glycol (DSG-PEG), PEG-DAG; PEG-PE; PEG-S-DAG; PEG-S-DMG; PEG-cer; a PEG-dialkyoxypropylcarbamate; 2-[(polyethylene glycol)-2000]-N,N-ditetradecylacetamide (ALC-0159); and combinations thereof.

In certain embodiments, the PEG has a high molecular weight, e.g., 2000-2400 g/mol. In certain embodiments, the PEG is PEG2000 (or PEG-2K). In certain embodiments, the PEGylated lipid herein is DMG-PEG2000, DSPE-PEG2000, DLPE-PEG2000, DSG-PEG2000, C8 PEG2000, or ALC-0159 (2-[(polyethylene glycol)-2000]-N,N-ditetradecylacetamide). In certain embodiments, the PEGylated lipid herein is DMG-PEG2000.

### C. Cholesterol-Based Lipids

The cholesterol component provides stability to the lipid bilayer structure within the nanoparticle. In some embodiments, the LNPs comprise one or more cholesterol-based lipids. Suitable cholesterol-based lipids include, for example: DC-Choi (N,N-dimethyl-N-ethylcarboxamidocholesterol), 1,4-bis(3-N-oleylamino-propyl) piperazine (Gao et al., Biochem Biophys Res Comm. (1991) 179:280; Wolf et al., BioTechniques (1997) 23:139; U.S. Pat. No. 5,744,335), imidazole cholesterol ester (“ICE”; WO2011/068810), sitosterol (22,23-dihydrostigmasterol), β-sitosterol, sitostanol, fucosterol, stigmasterol (stigmasta-5,22-dien-3-ol), ergosterol; desmosterol (3β-hydroxy-5,24-cholestadiene); lanosterol (8,24-lanostadien-3b-ol); 7-dehydrocholesterol (Δ5,7-cholesterol); dihydrolanosterol (24,25-dihydrolanosterol); zymosterol (5α-cholesta-8,24-dien-3β-ol); lathosterol (5α-cholest-7-en-3β-ol); diosgenin ((3β,25R)-spirost-5-en-3-ol); campesterol (campest-5-en-3β-ol); campestanol (5α-campestan-3b-ol); 24-methylene cholesterol (5,24 (28)-cholestadien-24-methylen-3β-ol); cholesteryl margarate (cholest-5-en-3β-yl heptadecanoate); cholesteryl oleate; cholesteryl stearate and other modified forms of cholesterol. In some embodiments, the cholesterol-based lipid used in the LNPs is cholesterol.

### D. Helper Lipids

A helper lipid enhances the structural stability of the LNP and helps the LNP in endosome escape. It improves uptake and release of the mRNA drug payload. In some embodiments, the helper lipid is a zwitterionic lipid, which has fusogenic properties for enhancing uptake and release of the drug payload. Examples of helper lipids are 1,2-dioleoyl-SN-glycero-3-phosphoethanolamine (DOPE); 1,2-distearoyl-sn-glycero-3-phosphocholine (DSPC); 1,2-dioleoyl-sn-glycero-3-phospho-L-serine (DOPS); 1,2-dielaidoyl-sn-glycero-3-phosphoethanolamine (DEPE); and 1,2-dioleoyl-sn-glycero-3-phosphocholine (DPOC), dipalmitoylphosphatidylcholine (DPPC), DMPC, 1,2-dilauroyl-sn-glycero-3-phosphocholine (DLPC), 1,2-Distearoylphosphatidylethanolamine (DSPE), and 1,2-dilauroyl-sn-glycero-3-phosphoethanolamine (DLPE).

Other exemplary helper lipids are dioleoylphosphatidylcholine (DOPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), phosphatidylserine, sphingolipids, sphingomyelins, ceramides, cerebrosides, gangliosides, 16-O-monomethyl PE, 16-O-dimethyl PE, 18-1-trans PE, 1-stearoyl-2-oleoyl-phosphatidyethanolamine (SOPE), or a combination thereof. In certain embodiments, the helper lipid is DOPE. In certain embodiments, the helper lipid is DSPC.

In various embodiments, the present LNPs comprise (i) a cationic lipid selected from OF-02, cKK-E10, GL-HEPES-E3-E10-DS-3-E18-1, GL-HEPES-E3-E12-DS-4-E10, GL-HEPES-E3-E12-DS-3-E14, IM-001 or IS-001; (ii) DMG-PEG2000; (iii) cholesterol; and (iv) DOPE.

In other embodiments, the present LNPs comprise (i) SM-102; (ii) DMG-PEG2000; (iii) cholesterol; and (iv) DSPC.

In yet other embodiments, the present LNPs comprise (i) ALC-0315; (ii) ALC-0159; (iii) cholesterol; and (iv) DSPC.

### E. Molar Ratios of the Lipid Components

The molar ratios of the above components are important for the LNPs' effectiveness in delivering mRNA. The molar ratio of the cationic lipid, the PEGylated lipid, the cholesterol-based lipid, and the helper lipid is A:B:C:D, where A+B+C+D=100%. In some embodiments, the molar ratio of the cationic lipid in the LNPs relative to the total lipids (i.e., A) is 35-55%, such as 35-50% (e.g., 38-42% such as 40%, or 45-50%). In some embodiments, the molar ratio of the PEGylated lipid component relative to the total lipids (i.e., B) is 0.25-2.75% (e.g., 1-2% such as 1.5%). In some embodiments, the molar ratio of the cholesterol-based lipid relative to the total lipids (i.e., C) is 20-50% (e.g., 27-30% such as 28.5%, or 38-43%). In some embodiments, the molar ratio of the helper lipid relative to the total lipids (i.e., D) is 5-35% (e.g., 28-32% such as 30%, or 8-12%, such as 10%). In some embodiments, the (PEGylated lipid+cholesterol) components have the same molar amount as the helper lipid. In some embodiments, the LNPs contain a molar ratio of the cationic lipid to the helper lipid that is more than 1.

In certain embodiments, the LNP of the disclosure comprises:
a cationic lipid at a molar ratio of 35% to 55% or 40% to 50% (e.g., a cationic lipid at a molar ratio of 35%, 36%, 37%, 38%, 39%, 40%, 41% 42%, 43%, 44%, 45%, 46%, 47%, 48%, 49%, 50%, 51%, 52%, 53%, 54%, or 55%);
a polyethylene glycol (PEG) conjugated (PEGylated) lipid at a molar ratio of 0.25% to 2.75% or 1.00% to 2.00% (e.g., a PEGylated lipid at a molar ratio of 0.25%, 0.50%, 0.75%, 1.00%, 1.25%, 1.50%, 1.75%, 2.00%, 2.25%, 2.50%, or 2.75%);
a cholesterol-based lipid at a molar ratio of 20% to 50%, 25% to 45%, or 28.5% to 43% (e.g., a cholesterol-based lipid at a molar ratio of 20%, 21%, 22%, 23%, 24%, 25%, 26%, 27%, 28%, 29%, 30%, 31%, 32%, 33%, 34%, 35%, 36%, 37%, 38%, 39%, 40%, 41% 42%, 43%, 44%, 45%, 46%, 47%, 48%, 49%, or 50%); and
a helper lipid at a molar ratio of 5% to 35%, 8% to 30%, or 10% to 30% (e.g., a helper lipid at a molar ratio of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35%),
wherein all of the molar ratios are relative to the total lipid content of the LNP.

In certain embodiments, the LNP comprises: a cationic lipid at a molar ratio of 40%; a PEGylated lipid at a molar ratio of 1.5%; a cholesterol-based lipid at a molar ratio of 28.5%; and a helper lipid at a molar ratio of 30%.

In certain embodiments, the LNP of the disclosure comprises: a cationic lipid at a molar ratio of 45 to 50%; a PEGylated lipid at a molar ratio of 1.5 to 1.7%; a cholesterol-based lipid at a molar ratio of 38 to 43%; and a helper lipid at a molar ratio of 9 to 10%.

In certain embodiments, the PEGylated lipid is dimyristoyl-PEG2000 (DMG-PEG2000).

In various embodiments, the cholesterol-based lipid is cholesterol.

In some embodiments, the helper lipid is 1,2-dioleoyl-SN-glycero-3-phosphoethanolamine (DOPE).

In certain embodiments, the LNP comprises: OF-02 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: cKK-E10 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E10-DS-3-E18-1 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-4-E10 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-3-E14 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: SM-102 at a molar ratio of 35% to 55%; DMG-PEG2000 at a molar ratio of 0.25% to 2.75%; cholesterol at a molar ratio of 20% to 50%; and DSPC at a molar ratio of 5% to 35%.

In certain embodiments, the LNP comprises: ALC-0315 at a molar ratio of 35% to 55%; ALC-0159 at a molar ratio of 0.25% to 2.75%; cholesterol at a molar ratio of 20% to 50%; and DSPC at a molar ratio of 5% to 35%.

In certain embodiments, the LNP comprises: OF-02 at a molar ratio of 40%; DMG-PEG2000 at a molar ratio of 1.5%; cholesterol at a molar ratio of 28.5%; and DOPE at a molar ratio of 30%. This LNP formulation is designated “Lipid A” herein.

In certain embodiments, the LNP comprises: cKK-E10 at a molar ratio of 40%; DMG-PEG2000 at a molar ratio of 1.5%; cholesterol at a molar ratio of 28.5%; and DOPE at a molar ratio of 30%. This LNP formulation is designated “Lipid B” herein.

In certain embodiments, the LNP comprises: GL-HEPES-E3-E10-DS-3-E18-1 at a molar ratio of 40%; DMG-PEG2000 at a molar ratio of 1.5%; cholesterol at a molar ratio of 28.5%; and DOPE at a molar ratio of 30%. This LNP formulation is designated “Lipid C” herein.

Typically, the LNP comprises: GL-HEPES-E3-E12-DS-4-E10 at a molar ratio of 40%; DMG-PEG2000 at a molar ratio of 1.5%; cholesterol at a molar ratio of 28.5%; and DOPE at a molar ratio of 30%. This LNP formulation is designated “Lipid D” herein.

In certain embodiments, the LNP comprises: GL-HEPES-E3-E12-DS-3-E14 at a molar ratio of 40%; DMG-PEG2000 at a molar ratio of 1.5%; cholesterol at a molar ratio of 28.5%; and DOPE at a molar ratio of 30%. This LNP formulation is designated “Lipid E” herein.

In certain embodiments, the LNP comprises: IM-001 at a molar ratio of 40%; DMG-PEG2000 at a molar ratio of 1.5%; cholesterol at a molar ratio of 28.5%; and DOPE at a molar ratio of 30%. This LNP formulation is designated “Lipid F” herein.

In certain embodiments, the LNP comprises: IS-001 at a molar ratio of 40%; DMG-PEG2000 at a molar ratio of 1.5%; cholesterol at a molar ratio of 28.5%; and DOPE at a molar ratio of 30%. This LNP formulation is designated “Lipid G” herein.

In certain embodiments, the LNP comprises: 9-heptadecanyl 8-{(2-hydroxyethyl) [6-oxo-6-(undecyloxy) hexyl]amino}octanoate (SM-102) at a molar ratio of 50%; 1,2-distearoyl-sn-glycero-3-phosphocholine (DSPC) at a molar ratio of 10%; cholesterol at a molar ratio of 38.5%; and 1,2-dimyristoyl-rac-glycero-3-methoxypolyethylene glycol-2000 (DMG-PEG2000) at a molar ratio of 1.5%.

In certain embodiments, the LNP comprises: (4-hydroxybutyl) azanediyl]di(hexane-6,1-diyl)bis(2-hexyldecanoate) (ALC-0315) at a molar ratio of 46.3%; 1,2-distearoyl-sn-glycero-3-phosphocholine (DSPC) at a molar ratio of 9.4%; cholesterol at a molar ratio of 42.7%; and 2-[(polyethylene glycol)-2000]-N,N-ditetradecylacetamide (ALC-0159) at a molar ratio of 1.6%.

In certain embodiments, the LNP comprises: (4-hydroxybutyl) azanediyl]di(hexane-6,1-diyl)bis(2-hexyldecanoate) (ALC-0315) at a molar ratio of 47.4%; 1,2-distearoyl-sn-glycero-3-phosphocholine (DSPC) at a molar ratio of 10%; cholesterol at a molar ratio of 40.9%; and 2-[(polyethylene glycol)-2000]-N,N-ditetradecylacetamide (ALC-0159) at a molar ratio of 1.7%.

To calculate the actual amount of each lipid to be put into an LNP formulation, the molar amount of the cationic lipid is first determined based on a desired N/P ratio, where N is the number of nitrogen atoms in the cationic lipid and P is the number of phosphate groups in the mRNA to be transported by the LNP. Next, the molar amount of each of the other lipids is calculated based on the molar amount of the cationic lipid and the molar ratio selected. These molar amounts are then converted to weights using the molecular weight of each lipid.

F. Nucleic Acids within LNPs

The LNP compositions described herein may comprise a nucleic acid (e.g., a mRNA) of the present invention.

Where desired, the LNP may be multi-valent. In some embodiments, the LNP may carry nucleic acids, such as mRNAs, that encode more than one polypeptide (antigen), such as two, three, four, five, or six polypeptides (antigens). For example, the LNP may carry multiple nucleic acids of the present invention (e.g., mRNA), each encoding a different polypeptide described herein; or carry a polycistronic mRNA that can be translated into more than one polypeptide described herein (e.g., each antigen-coding sequence is separated by a nucleotide linker encoding a self-cleaving peptide such as a 2A peptide). An LNP carrying different nucleic acids (e.g., mRNA) typically comprises (encapsulates) multiple copies of each nucleic acid. For example, an LNP carrying or encapsulating two different nucleic acids typically carries multiple copies of each of the two different nucleic acids.

Typically, two or more (e.g., two or three) nucleic acids (e.g., mRNAs) as described herein encoding different polypeptides as described herein are co-encapsulated in the same LNP. For example, the LNPs described herein may co-encapsulate a nucleic acid (e.g. mRNA) encoding a chimeric C. acnes DsA1/DsA2 polypeptide and a nucleic acid (e.g. mRNA) encoding a C. acnes PITP polypeptide. The LNPs described herein may co-encapsulate a nucleic acid (e.g. mRNA) encoding a chimeric C. acnes DsA1/DsA2 polypeptide and a nucleic acid (e.g. mRNA) encoding a modified C. acnes CAMP2 polypeptide (or a C. acnes CAMP2 polypeptide). The LNPs described herein may co-encapsulate a nucleic acid (e.g. mRNA) encoding a C. acnes PITP polypeptide and a nucleic acid (e.g. mRNA) encoding a modified C. acnes CAMP2 polypeptide (or a C. acnes CAMP2 polypeptide). Any two nucleic acids (e.g., two mRNAs) as described herein may be present in a weight ratio of 1:1.

The LNPs described herein may co-encapsulate (i) a nucleic acid (e.g. mRNA) encoding a chimeric C. acnes DsA1/DsA2 polypeptide as described herein, (ii) a nucleic acid (e.g. mRNA) encoding a C. acnes PITP polypeptide as described herein; and (iii) a nucleic acid (e.g. mRNA) encoding a modified C. acnes CAMP2 polypeptide as described herein (or a C. acnes CAMP2 polypeptide). Three nucleic acids (e.g., three mRNAs) as described herein may be present in a weight ratio of 1:1:1.

The LNP is as described herein (e.g. Lipid D).

Alternatively, any two or more (e.g., three) nucleic acids (e.g., mRNAs) encoding different polypeptides as described herein are encapsulated in separate LNPs. In some embodiments, a single LNP formulation may comprise multiple kinds (e.g., two, three, four, five, six, seven, eight, nine, ten, or more) of LNPs, each kind carrying a different nucleic acid (e.g., mRNA).

When the nucleic acid is mRNA, the mRNA may be unmodified (i.e., containing only natural ribonucleotides A, U, C, and/or G linked by phosphodiester bonds), or chemically modified (e.g., including nucleotide analogs such as pseudouridines (e.g., N-1-methyl pseudouridine), 2′-fluoro ribonucleotides, and 2′-methoxy ribonucleotides, and/or phosphorothioate bonds). The mRNA molecule may comprise a 5′ cap and a polyA tail.

### G. Buffer and Other Components

To stabilize the nucleic acid and/or LNPs (e.g., to prolong the shelf-life of the vaccine product), to facilitate administration of the LNP pharmaceutical composition, and/or to enhance in vivo expression of the nucleic acid, the nucleic acid and/or LNP can be formulated in combination with one or more carriers, targeting ligands, stabilizing reagents (e.g., preservatives and antioxidants), and/or other pharmaceutically acceptable excipients. Examples of such excipients are parabens, thimerosal, thiomersal, chlorobutanol, benzalkonium chloride, and chelators (e.g., EDTA).

The LNP compositions of the present disclosure can be provided as a frozen liquid form or a lyophilized form. A variety of cryoprotectants may be used, including, without limitations, sucrose, trehalose, glucose, mannitol, mannose, dextrose, and the like. The cryoprotectant may constitute 5-30% (w/v) of the LNP composition. In some embodiments, the LNP composition comprises trehalose, e.g., at 5-30% (e.g., 10%) (w/v). Once formulated with the cryoprotectant, the LNP compositions may be frozen (or lyophilized and cryopreserved) at −20° C. to −80° C.

The LNP compositions may be provided to a patient in an aqueous buffered solution-thawed if previously frozen, or if previously lyophilized, reconstituted in an aqueous buffered solution at bedside. The buffered solution preferably is isotonic and suitable for e.g., intramuscular or intradermal injection. In some embodiments, the buffered solution is a phosphate-buffered saline (PBS).

### Nucleic Acids

A nucleic acid of the invention may be RNA or DNA. The nucleic acids of the invention may be single or double-stranded. In certain embodiments, the nucleic acid is RNA, e.g. mRNA.

mRNA

In some embodiments, the nucleic acids of the present invention are messenger RNAs (mRNAs). mRNAs can be modified or unmodified. mRNAs may contain one or more coding and non-coding regions. A coding region is alternatively referred to as an open reading frame (ORF). Non-coding regions in an mRNA include the 5′ cap, 5′ untranslated region (UTR), 3′ UTR, and a polyA tail. An mRNA can be purified from natural sources, produced using recombinant expression systems (e.g., in vitro transcription) and optionally purified, or chemically synthesised.

In certain embodiments, the mRNA comprises an ORF encoding an antigen of interest. In certain embodiments, the RNA (e.g., mRNA) further comprises at least one 5′ UTR, 3′ UTR, a poly(A) tail, and/or a 5′ cap. In some embodiments, the mRNA comprises (i) a 5′ cap as defined herein; (ii) a 5′ untranslated region (UTR) as defined herein; (iii) a protein coding region; (iv) a 3′ UTR as defined herein; and (v) a polyA tail. Typically, the 3′ end of (i) bonds directly to the 5′ end of (ii) via a 3′ to 5′ phosphodiester linkage; the 3′ end of (ii) bonds directly to the 5′ end of (iii) via a 3′ to 5′ phosphodiester linkage; the 3′ end of (iii) bonds directly to the 5′ end of (iv) via a 3′ to 5′ phosphodiester linkage; and the 3′ end of (iv) bonds directly to the 5′ end of (v) via a 3′ to 5′ phosphodiester linkage.

In certain embodiments, the mRNA comprises at least one, at least two, at least three or more stop codon(s), wherein the stop codon(s) can be selected from UAA, UGA and UAG, and wherein the at least two, at least three or more stop codon(s) can be identical or different. Typically, the at least one stop codon comprises UAA or UGA (e.g. UAA). Typically, the at least two stop codons comprise at least two identical stop codons, such as UAA or UGA (e.g. UAAUAA or UGAUGA) or at least two different stop codons, which may in particular be selected from UAA and UGA (e.g. UGAUAA). Typically, the at least three stop codons comprise UAA, UGA and UAG (e.g. UGAUAAUAG).

An mRNA sequence is presented in the 5′ to 3′ direction unless otherwise indicated.

### 5′ Cap

An mRNA 5′ cap can provide resistance to nucleases found in most eukaryotic cells and promote translation efficiency. Several types of 5′ caps are known. A 7-methylguanosine cap (also referred to as “m7G” or “Cap-0”), comprises a guanosine that is linked through a 5′-5′-triphosphate bond to the first transcribed nucleotide.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp, (5′(A,G(5′)ppp(5′)A, and G(5′)ppp(5′)G. Additional cap structures are described in U.S. Publication No. US 2016/0032356 and U.S. Publication No. US 2018/0125989, which are incorporated herein by reference.

5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′)G (the ARCA cap); G(5′)ppp(5′)A; G(5′)ppp(5′)G; m7G(5′)ppp(5′)A; m7G(5′)ppp(5′)G; m7G(5′)ppp(5′)(2′oMeA)pG; m7G(5′)ppp(5′)(2′oMeA)pU; m7G(5′)ppp(5′)(2′oMeG)pG (New England BioLabs, Ipswich, MA; TriLink Biotechnologies). 5′-capping of modified RNA may be completed post-transcriptionally using a vaccinia virus capping enzyme to generate the Cap 0 structure: m7G(5′)ppp(5′)G. Cap 1 structure may be generated using both vaccinia virus capping enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase.

In certain embodiments, the mRNA of the disclosure comprises a 5′ cap selected from the group consisting of 3′-O-Me-m7G(5′)ppp(5′)G (the ARCA cap), G(5′)ppp(5′)A, G(5′)ppp(5′)G, m7G(5′)ppp(5′)A, m7G(5′)ppp(5′)G, m7G(5′)ppp(5′) (2′OMeA) pG, m7G(5′)ppp(5′) (2′OMeA)pU, and m7G(5′)ppp(5′) (2′OMeG)pG.

In certain embodiments, the mRNA of the disclosure comprises a 5′ cap of:

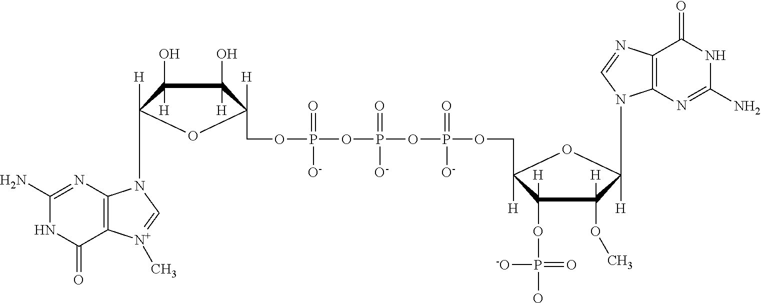

### Untranslated Region (UTR)

In some embodiments, the mRNA of the invention includes a 5′ and/or 3′ untranslated region (UTR). In mRNA, the 5′ UTR starts at the transcription start site and continues to the start codon but does not include the start codon. The 3′ UTR starts immediately following the stop codon and continues until the transcriptional termination signal.

In some embodiments, the mRNA disclosed herein may comprise a 5′ UTR that includes one or more elements that affect an mRNA's stability or translation. In some embodiments, a 5′ UTR may be about 10 to 5,000 nucleotides in length. In some embodiments, a 5′ UTR may be about 50 to 500 nucleotides in length. In some embodiments, the 5′ UTR is at least about 10 nucleotides in length, about 20 nucleotides in length, about 30 nucleotides in length, about 40 nucleotides in length, about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length or about 5,000 nucleotides in length.

In some embodiments, the mRNA disclosed herein may comprise a 3′ UTR comprising one or more of a polyadenylation signal, a binding site for proteins that affect an mRNA's stability of location in a cell, or one or more binding sites for miRNAs. In some embodiments, a 3′ UTR may be 50 to 5,000 nucleotides in length or longer. In some embodiments, a 3′ UTR may be 50 to 1,000 nucleotides in length or longer. In some embodiments, the 3′ UTR is at least about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length.

In some embodiments, the mRNA disclosed herein may comprise a 5′ or 3′ UTR that is derived from a gene distinct from the one encoded by the mRNA transcript (i.e., the UTR is a heterologous UTR).

In certain embodiments, the 5′ and/or 3′ UTR sequences can be derived from mRNA which are stable (e.g., globin, actin, GAPDH, tubulin, histone, or citric acid cycle enzymes) to increase the stability of the mRNA. For example, a 5′ UTR sequence may include a partial sequence of a CMV immediate-early 1 (IE1) gene, or a fragment thereof, to improve the nuclease resistance and/or improve the half-life of the mRNA. Also contemplated is the inclusion of a sequence encoding human growth hormone (hGH), or a fragment thereof, to the 3′ end or untranslated region of the mRNA. Generally, these modifications improve the stability and/or pharmacokinetic properties (e.g., half-life) of the mRNA relative to their unmodified counterparts, and include, for example, modifications made to improve such mRNA resistance to in vivo nuclease digestion.

Exemplary 5′ UTRs include a sequence derived from a CMV immediate-early 1 (IE1) gene (U.S. Publication Nos. 2014/0206753 and 2015/0157565, each of which is incorporated herein by reference), or the sequence GGGAUCCUACC (SEQ ID NO: 264) (U.S. Publication No. 2016/0151409, incorporated herein by reference).

In various embodiments, the 5′ UTR may be derived from the 5′ UTR of a TOP gene. TOP genes are typically characterized by the presence of a 5′-terminal oligopyrimidine (TOP) tract. Furthermore, most TOP genes are characterized by growth-associated translational regulation. However, TOP genes with a tissue specific translational regulation are also known. In certain embodiments, the 5′ UTR derived from the 5′ UTR of a TOP gene lacks the 5′ TOP motif (the oligopyrimidine tract) (e.g., U.S. Publication Nos. 2017/0029847, 2016/0304883, 2016/0235864, and 2016/0166710, each of which is incorporated herein by reference).

In certain embodiments, the 5′ UTR is derived from a ribosomal protein Large 32 (L32) gene (U.S. Publication No. 2017/0029847, supra).

In certain embodiments, the 5′ UTR is derived from the 5′ UTR of an hydroxysteroid (17-b) dehydrogenase 4 gene (HSD17B4) (U.S. Publication No. 2016/0166710, supra).

In certain embodiments, the 5′ UTR is derived from the 5′ UTR of an ATP5A1 gene (U.S. Publication No. 2016/0166710, supra).

In some embodiments, an internal ribosome entry site (IRES) is used instead of a 5′ UTR.

In some embodiments, the 5′UTR comprises a nucleic acid sequence set forth in SEQ ID NO: 265 and reproduced below:

(SEQ ID NO: 265)
GGACAGAUCGCCUGGAGACGCCAUCCACGCUGUUUUGACCUCCAUAGAAG
ACACCGGGACCGAUCCAGCCUCCGCGGCCGGGAACGGUGCAUUGGAACGC
GGAUUCCCCGUGCCAAGAGUGACUCACCGUCCUUGACACG.

In some embodiments, the 3′UTR comprises a nucleic acid sequence set forth in SEQ ID NO: 266 and reproduced below:

(SEQ ID NO: 266)
CGGGUGGCAUCCCUGUGACCCCUCCCCAGUGCCUCUCCUGGCCCUGGAA
GUUGCCACUCCAGUGCCCACCAGCCUUGUCCUAAUAAAAUUAAGUUGCA
UC.

The 5′ UTR and 3′UTR are described in further detail in WO2012/075040, incorporated herein by reference.

### Polyadenylated Tail

As used herein, the terms “poly(A) sequence,” “poly(A) tail,” and “poly(A) region” refer to a sequence of adenosine nucleotides at the 3′ end of the mRNA molecule. The poly(A) tail may confer stability to the mRNA and protect it from exonuclease degradation. The poly(A) tail may enhance translation. In some embodiments, the poly(A) tail is essentially homopolymeric. For example, a poly(A) tail of 100 adenosine nucleotides may have essentially a length of 100 nucleotides. In certain embodiments, the poly(A) tail may be interrupted by at least one nucleotide different from an adenosine nucleotide (e.g., a nucleotide that is not an adenosine nucleotide). For example, a poly(A) tail of 100 adenosine nucleotides may have a length of more than 100 nucleotides (comprising 100 adenosine nucleotides and at least one nucleotide, or a stretch of nucleotides, that are different from an adenosine nucleotide). In certain embodiments, the poly(A) tail comprises the sequence

(SEQ ID NO: 267)
AAAAAAAAAAAAAAAAAAAAAAAAAAAAAAGCAUAUGACUAAAAAAAAAA
AAAAAAAAAAAAAAAAAAAAAAAAAAAAAAAAAAAAAAAAAAAAAAAAAA
AAAAAAAAAA.

The “poly(A) tail,” as used herein, typically relates to RNA. However, in the context of the disclosure, the term likewise relates to corresponding sequences in a DNA molecule (e.g., a “poly(T) sequence”).

The poly(A) tail may comprise about 10 to about 500 adenosine nucleotides, about 10 to about 200 adenosine nucleotides, about 40 to about 200 adenosine nucleotides, or about 40 to about 150 adenosine nucleotides. The length of the poly(A) tail may be at least about 10, 50, 75, 100, 150, 200, 250, 300, 350, 400, 450, or 500 adenosine nucleotides. In some embodiments, the polyA tail comprises at least 75 nucleotides (e.g. about 80 adenosine nucleotides). In some embodiments, the polyA tail comprises at least 100 adenosine nucleotides (e.g. about 115 adenosine nucleotides). Typically, the polyA tail comprises at least 100 adenosine nucleotides.

In some embodiments where the nucleic acid is an RNA, the poly(A) tail of the nucleic acid is obtained from a DNA template during RNA in vitro transcription. In certain embodiments, the poly(A) tail is obtained in vitro by common methods of chemical synthesis without being transcribed from a DNA template. In various embodiments, poly(A) tails are generated by enzymatic polyadenylation of the RNA (after RNA in vitro transcription) using commercially available polyadenylation kits and corresponding protocols, or alternatively, by using immobilized poly(A) polymerases, e.g., using methods and means as described in WO2016/174271.

The nucleic acid may comprise a poly(A) tail obtained by enzymatic polyadenylation, wherein the majority of nucleic acid molecules comprise about 100 (+/−20) to about 500 (+/−50) or about 250 (+/−20) adenosine nucleotides.

In some embodiments, the nucleic acid may comprise a poly(A) tail derived from a template DNA and may additionally comprise at least one additional poly(A) tail generated by enzymatic polyadenylation, e.g., as described in WO2016/091391.

In certain embodiments, the nucleic acid comprises at least one polyadenylation signal.

In various embodiments, the nucleic acid may comprise at least one poly(C) sequence.

The term “poly(C) sequence,” as used herein, is intended to be a sequence of cytosine nucleotides of up to about 200 cytosine nucleotides. In some embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In some embodiments, the poly(C) sequence comprises about 30 cytosine nucleotides.

### Chemical Modification

The mRNA disclosed herein may be modified or unmodified. In some embodiments, the mRNA may comprise at least one chemical modification. In some embodiments, the mRNA disclosed herein may contain one or more modifications that typically enhance RNA stability. Exemplary modifications can include backbone modifications, sugar modifications, or base modifications. In some embodiments, the disclosed mRNA may be synthesized from naturally occurring nucleotides and/or nucleotide analogues (modified nucleotides) including, but not limited to, purines (adenine (A) and guanine (G)) or pyrimidines (thymine (T), cytosine (C), and uracil (U)). In certain embodiments, the disclosed mRNA may be synthesized from modified nucleotide analogues or derivatives of purines and pyrimidines, such as, e.g., 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, phosphoramidates, phosphorothioates, peptide nucleotides, methylphosphonates, 7-deazaguanosine, 5-methylcytosine, and inosine.

In some embodiments, the disclosed mRNA may comprise at least one chemical modification including, but not limited to, pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In some embodiments, the chemical modification is selected from the group consisting of pseudouridine, N1-methylpseudouridine, 5-methylcytosine, 5-methoxyuridine, and a combination thereof.

In some embodiments, the chemical modification comprises N1-methylpseudouridine. Typically, the chemical modification comprises N1-methylpseudouridine in place of every uridine, i.e. 100% of U residues are N1-methylpseudouridine.

In some embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified.

In some embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified.

The preparation of such analogues is described, e.g., in U.S. Pat. Nos. 4,373,071, 4,401,796, 4,415,732, 4,458,066, 4,500,707, 4,668,777, 4,973,679, 5,047,524, 5,132,418, 5,153,319, 5,262,530, and 5,700,642.

mRNA Synthesis

The mRNAs disclosed herein may be synthesized according to any of a variety of methods. For example, mRNAs according to the present disclosure may be synthesized via in vitro transcription (IVT). Some methods for in vitro transcription are described, e.g., in Geall et al. (2013) Semin. Immunol. 25 (2): 152-159; Brunelle et al. (2013) Methods Enzymol. 530:101-14. Briefly, IVT is typically performed with a linear or circular DNA template containing a promoter, a pool of ribonucleotide triphosphates, a buffer system that may include DTT and magnesium ions, an appropriate RNA polymerase (e.g., T3, T7, or SP6 RNA polymerase), DNase I, pyrophosphatase, and/or RNase inhibitor. The exact conditions may vary according to the specific application. The presence of these reagents is generally undesirable in a final mRNA product and these reagents can be considered impurities or contaminants which can be purified or removed to provide a clean and/or homogeneous mRNA that is suitable for therapeutic use. While mRNA provided from in vitro transcription reactions may be desirable in some embodiments, other sources of mRNA can be used according to the instant disclosure including wild-type mRNA produced from bacteria, fungi, plants, and/or animals.

### Processes for Making the Present LNP Vaccines

The present LNPs can be prepared by various techniques presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion that results in the formation of MLVs. Unilamellar vesicles (ULV) can then be formed by homogenization, sonication or extrusion of the multilamellar vesicles. In addition, unilamellar vesicles can be formed by detergent removal techniques.

Various methods are described in US 2011/0244026, US 2016/0038432, US 2018/0153822, US 2018/0125989, and PCT/US2020/043223 (filed Jul. 23, 2020) and can be used to practice the present disclosure. One exemplary process entails encapsulating mRNA by mixing it with a mixture of lipids, without first pre-forming the lipids into lipid nanoparticles, as described in US 2016/0038432. Another exemplary process entails encapsulating mRNA by mixing pre-formed LNPs with mRNA, as described in US 2018/0153822.

In some embodiments, the process of preparing mRNA-loaded LNPs includes a step of heating one or more of the solutions to a temperature greater than ambient temperature, the one or more solutions being the solution comprising the pre-formed lipid nanoparticles, the solution comprising the mRNA and the mixed solution comprising the LNP-encapsulated mRNA. In some embodiments, the process includes the step of heating one or both of the mRNA solution and the pre-formed LNP solution, prior to the mixing step. In some embodiments, the process includes heating one or more of the solutions comprising the pre-formed LNPs, the solution comprising the mRNA and the solution comprising the LNP-encapsulated mRNA, during the mixing step. In some embodiments, the process includes the step of heating the LNP-encapsulated mRNA, after the mixing step. In some embodiments, the temperature to which one or more of the solutions is heated is or is greater than about 30° C., 37° C., 40° C., 45° C., 50° C., 55° C., 60° C., 65° C., or 70° C. In some embodiments, the temperature to which one or more of the solutions is heated ranges from about 25-70° C., about 30-70° C., about 35-70° C., about 40-70° C., about 45-70° C., about 50-70° C., or about 60-70° C. In some embodiments, the temperature is about 65° C.

Various methods may be used to prepare an mRNA solution suitable for the present disclosure. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water or a buffer at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, an mRNA stock solution is mixed with a buffer solution using a pump. Exemplary pumps include but are not limited to gear pumps, peristaltic pumps and centrifugal pumps. Typically, the buffer solution is mixed at a rate greater than that of the mRNA stock solution. For example, the buffer solution may be mixed at a rate at least 1×, 2×, 3×, 4×, 5×, 6×, 7×, 8×, 9×, 10×, 15×, or 20× greater than the rate of the mRNA stock solution. In some embodiments, a buffer solution is mixed at a flow rate ranging between about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute). In some embodiments, a buffer solution is mixed at a flow rate of, or greater than, about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging between about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

The process of incorporation of a desired mRNA into a lipid nanoparticle is referred to as “loading.” Exemplary methods are described in Lasic et al., FEBS Lett. (1992) 312:255-8. The LNP-incorporated nucleic acids may be completely or partially located in the interior space of the lipid nanoparticle, within the bilayer membrane of the lipid nanoparticle, or associated with the exterior surface of the lipid nanoparticle membrane. The incorporation of an mRNA into lipid nanoparticles is also referred to herein as “encapsulation” wherein the nucleic acid is entirely or substantially contained within the interior space of the lipid nanoparticle.

Suitable LNPs may be made in various sizes. In some embodiments, decreased size of lipid nanoparticles is associated with more efficient delivery of an mRNA. Selection of an appropriate LNP size may take into consideration the site of the target cell or tissue and to some extent the application for which the lipid nanoparticle is being made.

A variety of methods known in the art are available for sizing of a population of lipid nanoparticles. Preferred methods herein utilize Zetasizer Nano ZS (Malvern Panalytical) to measure LNP particle size. In one protocol, 10 μl of an LNP sample are mixed with 990 μl of 10% trehalose. This solution is loaded into a cuvette and then put into the Zetasizer machine. The z-average diameter (nm), or cumulants mean, is regarded as the average size for the LNPs in the sample. The Zetasizer machine can also be used to measure the polydispersity index (PDI) by using dynamic light scattering (DLS) and cumulant analysis of the autocorrelation function. Average LNP diameter may be reduced by sonication of formed LNP. Intermittent sonication cycles may be alternated with quasi-elastic light scattering (QELS) assessment to guide efficient lipid nanoparticle synthesis.

In some embodiments, the LNP has an average diameter of 30 nm to 200 nm (e.g. an average diameter of 80 nm to 150 nm).

In some embodiments, the majority of purified LNPs, i.e., greater than about 50%, 55%, 60%, 65%, 70%, 75%, 80%, 85%, 90%, 95%, 96%, 97%, 98%, or 99% of the LNPs, have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, substantially all (e.g., greater than 80 or 90%) of the purified lipid nanoparticles have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm).

In some embodiments, the LNPs in the present composition have an average size of less than 150 nm, less than 120 nm, less than 100 nm, less than 90 nm, less than 80 nm, less than 70 nm, less than 60 nm, less than 50 nm, less than 30 nm, or less than 20 nm.

In some embodiments, greater than about 70%, 75%, 80%, 85%, 90%, 95%, 96%, 97%, 98%, 99% of the LNPs in the present composition have a size ranging from about 40-90 nm (e.g., about 45-85 nm, about 50-80 nm, about 55-75 nm, about 60-70 nm) or about 50-70 nm (e.g., 55-65 nm) are particular suitable for pulmonary delivery via nebulization.

In some embodiments, the dispersity, or measure of heterogeneity in size of molecules (PDI), of LNPs in a pharmaceutical composition provided by the present disclosure is less than about 0.5. In some embodiments, an LNP has a PDI of less than about 0.5, less than about 0.4, less than about 0.3, less than about 0.28, less than about 0.25, less than about 0.23, less than about 0.20, less than about 0.18, less than about 0.16, less than about 0.14, less than about 0.12, less than about 0.10, or less than about 0.08. The PDI may be measured by a Zetasizer machine as described above.

In some embodiments, greater than about 75%, 80%, 85%, 90%, 95%, 96%, 97%, 98%, or 99% of the purified LNPs in a pharmaceutical composition provided herein encapsulate an mRNA within each individual particle. In some embodiments, substantially all (e.g., greater than 80% or 90%) of the purified lipid nanoparticles in a pharmaceutical composition encapsulate an mRNA within each individual particle. In some embodiments, a lipid nanoparticle has an encapsulation efficiency of between 50% and 99%; or greater than about 60, 65, 70, 75, 80, 85, 90, 92, 95, 98, or 99%. Typically, lipid nanoparticles for use herein have an encapsulation efficiency of at least 90% (e.g., at least 91, 92, 93, 94, or 95%).

In some embodiments, an LNP has a N/P ratio of between 1 and 10. In some embodiments, a lipid nanoparticle has a N/P ratio above 1, about 1, about 2, about 3, about 4, about 5, about 6, about 7, or about 8. In further embodiments, a typical LNP herein has an N/P ratio of 4.

In some embodiments, a pharmaceutical composition according to the present disclosure contains at least about 0.5μ, 1μ, 5μ, 10μ, 100μ, 500 μg, or 1000μ g of encapsulated mRNA. In some embodiments, a pharmaceutical composition contains about 0.1 μg to 1000 μg, at least about 0.5 μg, at least about 0.8 μg, at least about 1 μg, at least about 5 μg, at least about 8 μg, at least about 10 μg, at least about 50 μg, at least about 100 μg, at least about 500 μg, or at least about 1000 μg of encapsulated mRNA.

In some embodiments, mRNA can be made by chemical synthesis or by in vitro transcription (IVT) of a DNA template. In this process, in an IVT process, a cDNA template is used to produce an mRNA transcript and the DNA template is degraded by a DNase. The transcript is purified by depth filtration and tangential flow filtration (TFF). The purified transcript is further modified by adding a cap and a tail, and the modified RNA is purified again by depth filtration and TFF.

The mRNA is then prepared in an aqueous buffer and mixed with an amphiphilic solution containing the lipid components of the LNPs. An amphiphilic solution for dissolving the four lipid components of the LNPs may be an alcohol solution. In some embodiments, the alcohol is ethanol. The aqueous buffer may be, for example, a citrate, phosphate, acetate, or succinate buffer and may have a pH of about 3.0-7.0, e.g., about 3.5, about 4.0, about 4.5, about 5.0, about 5.5, about 6.0, or about 6.5. The buffer may contain other components such as a salt (e.g., sodium, potassium, and/or calcium salts). In particular embodiments, the aqueous buffer has 1 mM citrate, 150 mM NaCl, pH 4.5.

The process for making a composition comprising LNPs and mRNA(s) involves mixing of a buffered mRNA solution with a solution of lipids in ethanol in a controlled homogeneous manner, where the ratio of lipids:mRNA is maintained throughout the mixing process. In this illustrative example, the mRNA is presented in an aqueous buffer containing citric acid monohydrate, tri-sodium citrate dihydrate, and sodium chloride. The mRNA solution is added to the solution (1 mM citrate buffer, 150 mM NaCl, pH 4.5). The lipid mixture of four lipids (e.g., a cationic lipid, a PEGylated lipid, a cholesterol-based lipid, and a helper lipid) is dissolved in ethanol. The aqueous mRNA solution and the ethanol lipid solution are mixed at a volume ratio of 4:1 in a “T” mixer with a near “pulseless” pump system. The resultant mixture is then subjected for downstream purification and buffer exchange. The buffer exchange may be achieved using dialysis cassettes or a TFF system. TFF may be used to concentrate and buffer-exchange the resulting nascent LNP immediately after formation via the T-mix process. The diafiltration process is a continuous operation, keeping the volume constant by adding appropriate buffer at the same rate as the permeate flow.

### Vectors

In one aspect, disclosed herein are vectors comprising the mRNA compositions disclosed herein. The RNA sequences encoding a protein of interest (e.g., mRNA encoding a polypeptide disclosed herein) can be cloned into a number of types of vectors. For example, the nucleic acids can be cloned into a vector including, but not limited to, a plasmid, a phagemid, a phage derivative, an animal virus, and a cosmid. Vectors of particular interest can include expression vectors, replication vectors, probe generation vectors, sequencing vectors, and vectors optimized for in vitro transcription.

In certain embodiments, the vector can be used to express mRNA in a host cell. In various embodiments, the vector can be used as a template for IVT. The construction of optimally translated IVT mRNA suitable for therapeutic use is disclosed in detail in Sahin, et al. (2014). Nat. Rev. Drug Discov. 13, 759-780; Weissman (2015). Expert Rev. Vaccines 14, 265-281.

In some embodiments, the vectors disclosed herein can comprise at least the following, from 5′ to 3′: an RNA polymerase promoter; a polynucleotide sequence encoding a 5′ UTR; a polynucleotide sequence encoding an ORF; a polynucleotide sequence encoding a 3′ UTR; and a polynucleotide sequence encoding at least one RNA aptamer. In some embodiments, the vectors disclosed herein may comprise a polynucleotide sequence encoding a poly(A) sequence and/or a polyadenylation signal.

A variety of RNA polymerase promoters are known. In some embodiments, the promoter can be a T7 RNA polymerase promoter. Other useful promoters can include, but are not limited to, T3 and SP6 RNA polymerase promoters. Consensus nucleotide sequences for T7, T3, and SP6 promoters are known.

Also disclosed herein are host cells (e.g., mammalian cells, e.g., human cells) comprising the vectors or RNA compositions disclosed herein. A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid or vector disclosed herein.

Vectors can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg, Germany), cationic liposome mediated transfection using lipofection, polymer encapsulation, peptide mediated transfection, biolistic particle delivery systems such as “gene guns” (see, for example, Nishikawa, et al. (2001). Hum Gene Ther. 12 (8): 861-70, or the TransIT-RNA transfection Kit (Mirus, Madison, WI).

Chemical means for introducing a vector into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present disclosure, in order to confirm the presence of the mRNA sequence in the host cell a variety of assays may be performed.

### Self-Replicating RNA, Trans-Replicating RNA and Non-Replicating RNA

Typically, the nucleic acid molecules described herein are non-replicating RNAs. However, the nucleic acid molecules described herein may alternatively be self-replicating RNAs or trans-replicating RNAs.

### Self-Replicating RNA

Self-replicating (or self-amplifying) RNA can be produced by using replication elements derived from, e.g., alphaviruses, and substituting the structural viral proteins with a nucleotide sequence encoding a protein of interest (e.g., a polypeptide described herein). A self-replicating RNA is typically a positive-strand molecule which can be directly translated after delivery to a cell, and this translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded antigen, or may be transcribed to provide further transcripts with the same sense as the delivered RNA which are translated to provide in situ expression of the antigen. The overall result of this sequence of transcriptions is a large amplification in the number of the introduced replicon RNAs and so the encoded antigen becomes a major polypeptide product of the cells.

One suitable system for achieving self-replication in this manner is to use an alphavirus-based replicon. These replicons are positive stranded (positive sense-stranded) RNAs which lead to translation of a replicase (or replicase-transcriptase) after delivery to a cell. The replicase is translated as a polyprotein which auto-cleaves to provide a replication complex which creates genomic-strand copies of the positive-strand delivered RNA. These negative (−)-stranded transcripts can themselves be transcribed to give further copies of the positive-stranded parent RNA and also to give a subgenomic transcript which encodes the antigen. Translation of the subgenomic transcript thus leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used, e.g., the attenuated TC83 mutant of VEEV has been used in replicons, see the following reference: WO2005/113782, incorporated herein by reference.

In one embodiment, each self-replicating RNA described herein encodes (i) an RNA-dependent RNA polymerase which can transcribe RNA from the self-replicating RNA molecule and (ii) an influenza protein antigen. The polymerase can be an alphavirus replicase, e.g., comprising one or more of alphavirus proteins nsP1, nsP2, nsP3, and nsP4. Whereas natural alphavirus genomes encode structural virion proteins in addition to the non-structural replicase polyprotein, in certain embodiments, the self-replicating RNA molecules do not encode alphavirus structural proteins. Thus, the self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the present disclosure and their place is taken by gene(s) encoding the immunogen of interest, such that the subgenomic transcript encodes the immunogen rather than the structural alphavirus virion proteins. Self-replicating RNA are described in further detail in WO2011005799, incorporated herein by reference.

### Trans-Replicating RNA

Trans-replicating (or trans-amplifying) RNA possess similar elements as the self-replicating RNA described above. However, with trans replicating RNA, two separate RNA molecules are used. A first RNA molecule encodes for the RNA replicase described above (e.g., the alphavirus replicase) and a second RNA molecule encodes for the protein of interest (e.g., a polypeptide described herein). The RNA replicase may replicate one or both of the first and second RNA molecule, thereby greatly increasing the copy number of RNA molecules encoding the protein of interest. Trans replicating RNA are described in further detail in WO2017162265, incorporated herein by reference.

### Non-Replicating RNA

Non-replicating (or non-amplifying) RNA is an RNA without the ability to replicate itself.

### Therapeutic Uses

In another aspect, the invention provides the polypeptides, nucleic acids, combinations or compositions of the present invention for use as a medicament. The invention also provides the use of the polypeptides, nucleic acids, combinations or compositions of the present invention for the manufacture of a medicament. The medicament may be used for treating or preventing a disease as described herein. The invention further provides a method of treating or preventing a disease comprising administering the polypeptides, nucleic acids, combinations or compositions of the present invention to a subject in need thereof. The polypeptides, nucleic acids, combinations or compositions of the present invention may, for example, be administered in an amount effective to treat or prevent the disease in the subject. Polypeptides, nucleic acids, combinations or compositions may thus be administered in an effective amount.

In another aspect, the invention provides the polypeptides, nucleic acids, combinations or compositions of the present invention for use in treating or preventing C. acnes infection in a subject (e.g. human). The invention also provides the use of the polypeptides, nucleic acids, combinations or compositions of the present invention for the manufacture of a medicament for treating or preventing C. acnes infection in a subject (e.g. human). The invention further provides a method of treating or preventing C. acnes infection in a subject (e.g. human), the method comprising administering the polypeptides, nucleic acids, combinations or compositions of the present invention to the subject. The polypeptides, nucleic acids, combinations or compositions of the present invention may, for example, be administered in an amount effective to treat or prevent C. acnes infection in the subject (i.e. administered in an effective amount). C. acnes infection may be mild, moderate or severe (e.g. moderate or severe).

The polypeptides, nucleic acids, combinations or compositions of the invention may be used for eliciting an immune response in a subject, e.g. an immune response against C. acnes infection.

In another aspect, the invention provides the polypeptides, nucleic acids, combinations or compositions of the present invention for use in treating or preventing acne (also known as acne vulgaris) in a subject (e.g. human). The invention also provides the use of the polypeptides, nucleic acids, combinations or compositions of the present invention for the manufacture of a medicament for treating or preventing acne in a subject (e.g. human). The invention further provides a method of treating or preventing acne in a subject (e.g. human), the method comprising administering the polypeptides, nucleic acids, combinations or compositions of the present invention to the subject. The polypeptides, nucleic acids, combinations or compositions of the present invention may, for example, be administered in an amount effective to treat or prevent acne in the subject (i.e. administered in an effective amount). Acne may be caused by C. acnes. Acne may be mild, moderate or severe (e.g. moderate or severe).

In some embodiments, a treatment as described herein achieves at least a 5%, at least a 10%, at least a 15%, at least a 20%, at least a 25%, at least a 30%, at least a 35%, at least a 40%, at least a 45%, at least a 50%, at least a 55%, at least a 60%, at least a 65%, at least a 70%, at least a 75%, at least a 80%, least a 85% or at least a 90% (e.g. at least a 50%) reduction in the number of inflammatory acne lesions relative to the number of inflammatory acne lesions prior to the treatment, e.g. at 2, 3, 4, 5, 6, 7, 8, 9, 10, 11, 12 months (e.g. 3 or 6 months) after administration of the treatment (e.g. after the last dose of the treatment). In some embodiments, the inflammatory acne lesions are on a subject's face. In some embodiments, the inflammatory acne lesions are on a subject's face and one other body area (e.g. chest or back).

In some embodiments, a treatment as described herein achieves at least a 5%, at least a 10%, at least a 15%, at least a 20%, at least a 25%, at least a 30%, at least a 35%, at least a 40%, at least a 45%, at least a 50%, at least a 55%, at least a 60%, at least a 65%, at least a 70%, at least a 75%, at least a 80%, least a 85% or at least a 90% (e.g. at least a 50%) reduction in the number of non-inflammatory acne lesions relative to the number of non-inflammatory acne lesions prior to the treatment, e.g. at 2, 3, 4, 5, 6, 7, 8, 9, 10, 11, 12 months (e.g. 3 or 6 months) after administration of the treatment (e.g. after the last dose of the treatment). In some embodiments, the non-inflammatory acne lesions are on a subject's face. In some embodiments, the non-inflammatory acne lesions are on a subject's face and one other body area (e.g. chest or back).

In some embodiments, a treatment as described herein achieves a reduction in the Investigator's Global Assessment (IGA) score for a subject relative to the score prior to the treatment, e.g. at 2, 3, 4, 5, 6, 7, 8, 9, 10, 11, 12 months (e.g. 3 or 6 months) after administration of the treatment (e.g. after the last dose of the treatment). In some embodiments, a treatment as described herein achieves at least a two-grade improvement in the Investigator's Global Assessment (IGA) score for a subject relative to the score prior to the treatment, e.g. at 2, 3, 4, 5, 6, 7, 8, 9, 10, 11, 12 months (e.g. 3 or 6 months) after administration of the treatment (e.g. after the last dose of the treatment). In some embodiments, the IGA score improvement results in an IGA score of 0, 1 or 2 (e.g. 0 or 1), e.g. at 2, 3, 4, 5, 6, 7, 8, 9, 10, 11, 12 months (e.g. 3 or 6 months) after administration of the treatment (e.g. after the last dose of the treatment).

The polypeptides, nucleic acids, combinations or compositions of the invention may be used for treating or preventing chronic blepharitis associated with C. acnes infection and/or endophthalmitis associated with C. acnes infection.

The invention provides the polypeptides, nucleic acids, combinations or compositions of the present invention for use in a method of providing protective immunity against a C. acnes infection in a subject. The invention also provides the use of the polypeptides, nucleic acids, combinations or compositions of the present invention for the manufacture of a medicament for use in a method of providing protective immunity against a C. acnes infection in a subject. The invention further provides a method of providing protective immunity against a C. acnes infection in a subject, the method comprising administering the polypeptides, nucleic acids, combinations or compositions of the present invention to the subject. The polypeptides, nucleic acids, combinations or compositions of the present invention may, for example, be administered in an amount effective to providing protective immunity against a C. acnes infection in the subject (i.e. administered in an effective amount). Protective immunity may be protective against development of a pathological condition induced by C. acnes infection (e.g. acne). Protective immunity may prevent or reduce development of a C. acnes-associated indication.

The polypeptides, nucleic acids, combinations or compositions of the invention may elicit antibodies (e.g. antigen-specific antibodies), such as IgG. Antibodies (DsA1, DsA2 and/or PITP-specific antigens) may bind the surface of C. acnes bacteria. Antibodies (DsA1, DsA2 and/or PITP-specific antigens) may opsonize C. acnes bacteria. This may allow recruitment of immune effector cells (e.g. phagocytes) to the bacteria. C. acnes bacteria may be phagocytosed by recruited immune effector cells. Within the IgG isotype, antibodies may be IgG1, IgG2, IgG3 or IgG4 subclass. The antibody may have a k or a 2 light chain. In some embodiments, the polypeptides, nucleic acids, combinations or compositions of the invention may elicit an antibody response that is cross-reactive against two or more strains or phylotypes of C. acnes, e.g. cross-reactive against two or more phylotypes (e.g. cross-reactive against phylotypes IA1, IA2, IB IC, II and III).

The polypeptides, nucleic acids, combinations or compositions of the invention may reduce inflammation associated with (e.g. caused by) C. acnes infection. The polypeptides, nucleic acids, combinations or compositions of the invention may reduce C. acnes-mediated tissue inflammation.

The polypeptides, nucleic acids, combinations or compositions of the invention may inhibit biofilm formation by C. acnes. In some embodiments, biofilm formation may be prevented. In some embodiments, biofilm formation may be reduced.

Administration of a polypeptide, nucleic acid, combination or composition of the invention to a subject may enable the subject to produce a C. acnes antigen-responsive memory B cell population on exposure to C. acnes bacteria or the C. acnes antigen. The polypeptides, nucleic acids, combinations or compositions of the invention may be used to induce a primary immune response and/or to boost an immune response.

The polypeptides, nucleic acids, combinations or compositions of the invention may be used in a prime-boost vaccination regime. Immunity against a C. acnes infection according to the invention may be provided by administering a priming vaccine, comprising a polypeptide, nucleic acid, combinations or composition of the invention, followed by a booster vaccine. The booster vaccine may be the same as the primer vaccine.

In certain embodiments, the subject is a vertebrate, e.g., a mammal, such as a human or a veterinary mammal (e.g. cat, dog, horse, cow, sheep, cattle, deer, goat, pig, rodents (e.g. mice)). In preferred embodiments, the subject is a human. The subject (e.g. the human subject) may be male or female. The subject may be a child (0-10 years old), an adolescent (10-18 years old) or an adult (over 18 years old). In some embodiments, human subjects may be 0-30, or 0-45 (e.g. 5-18, 12-45, 18-45 or 9-45) years old. For example, the subject may be 9-45, 12-45 or 18-45 (e.g. 9-45) years old.

In some embodiments, the subject has at least one of the following: (i) score of grade 3 or grade 4 on the IGA scale; (ii) at least 25 non-inflammatory lesions (e.g. open and/or closed comedones) optionally on the subject's face; (iii) at least 20 inflammatory lesions (e.g. papules and/or pustules) optionally on the subject's face; (iv) 2 or fewer nodulocystic lesions (e.g. nodules and/or cysts) optionally on the subject's face. In some embodiments, the subject has at least two of (i)-(iv). In some embodiments, the subject has at least three of (i)-(iv). Typically, the subject has all four of (i)-(iv). A subject having all four of (i)-(iv) may have moderate/severe acne.

Acne vulgaris (also referred to herein as acne) manifests in different severity grades: mild, moderate and severe. Moderate and severe acne account for more than one third of all cases and require medical treatment. In some embodiments, subjects may have mild, moderate or severe (e.g., moderate or severe acne) C. acnes infection. The subjects may have mild, moderate or severe acne (e.g., moderate or severe acne). In some embodiments, the subjects have mild, moderate or severe acne (e.g., moderate or severe acne).

### Modes of Administration

The polypeptides, nucleic acids, combinations or compositions of the present invention can be administered parenterally (e.g., intramuscularly, intradermally, subcutaneously, intraperitoneally, intravenously, or to the interstitial space of a tissue) or by rectal, oral, vaginal, topical, transdermal, intranasal, sublingual, ocular, aural, pulmonary or other mucosal administration. In some embodiments, delivery is by mucosal administration. Typically, administration is intramuscular.

In certain embodiments, nucleic acids, polypeptides, compositions or combinations (e.g., compositions) of the invention are provided for use in intramuscular (IM) injection. The nucleic acids, polypeptides, compositions or combinations (e.g., compositions) can be administered to the thigh or the upper arm of a subject at, e.g., their deltoid muscle in the upper arm. In some embodiments, the nucleic acids, polypeptides, compositions or combinations (e.g., compositions) are provided in a pre-filled syringe or injector (e.g., single-chambered or multi-chambered). Injection may be via a needle (e.g. a hypodermic needle), but needle-free injection may alternatively be used. A typical intramuscular dose is 0.5 ml. In some embodiments, the nucleic acids, polypeptides, compositions or combinations (e.g., compositions) are provided for use in inhalation and is provided in a pre-filled pump, aerosolizer, or inhaler.

In certain embodiments, nucleic acids, polypeptides, compositions or combinations (e.g., compositions) of the invention are provided for use in skin injection, e.g. in the epidermis, the dermis or the hypodermis of the skin. In some embodiments, the nucleic acids, polypeptides, compositions or combinations of the invention (e.g. compositions) are provided in a device suitable for skin injection, such as a needle (e.g. an epidermic, dermic or hypodermic needle), a needle free device, a microneedle device or a microprojection array device. Examples of microneedle or microprojection array devices suitable for the skin injection according to the invention are described in US20230270842A1, US20220339416A1, US20210085598A1, US20200246450A1, US20220143376A1, US20180264244A1, US20180263641A1, US20110245776A1.

The nucleic acids, polypeptides, compositions or combinations (e.g., compositions) of the invention may be used to elicit systemic, cutaneous and/or mucosal immunity.

Dosage treatment can be a single dose schedule or a multiple dose schedule. Multiple doses (e.g. two or three) may be used in a primary immunisation schedule and/or in a booster immunisation schedule. A primary dose schedule may be followed by a booster dose schedule. Multiple doses (e.g., two doses or three) will typically be administered at least 1 week apart (e.g. about 2 weeks, about 3 weeks, about 4 weeks, about 6 weeks, about 8 weeks, about 10 weeks, about 12 weeks, about 16 weeks, etc.) to subjects in need thereof to achieve the desired therapeutic or prophylactic effects (e.g., two months). The doses (e.g., prime and booster doses) may be separated by an interval of e.g., 1 week, 2 weeks, 3 weeks, 4 weeks, one month, two months, three months, four months, five months, six months, one year, two years, five years, or ten years (e.g., two months). In some embodiments, a subject is administered a single dose intramuscularly. In some embodiments, the subject is administered two doses intramuscularly (e.g. two months apart).

A composition of the invention may be in the form of an extemporaneous formulation, e.g. a composition of the invention may be lyophilised. Such compositions may be reconstituted with a physiological buffer (e.g., PBS) just before use. The compositions of the invention may be provided in the form of an aqueous solution or a frozen aqueous solution and can be directly administered to subjects without reconstitution (after thawing, if previously frozen).

In some embodiments of a composition comprising one or more nucleic acids (e.g., mRNA(s)) as described herein, a single dose of the composition contains 1-300 (or 1-50) μg of a mRNA as described herein (e.g., monovalent or multivalent). For example, a single dose may contain about 2.5 μg, about 5μ, about 7.5μ, about 10μ, about 12.5μ, about 15μ, about 30μ, about 45μ, about 60μ g, about 75 μg, about 90 μg, about 105 μg, about 120 μg, about 135 μg, about 150 μg, about 165 μg, about 180 μg, about 195 μg, about 210 μg, about 225 μg, about 240 μg, about 250 μg, about 260 μg, about 275 μg, or about 300 μg of the one or more nucleic acids (e.g., mRNA(s)) described herein, e.g. for intramuscular (IM) injection. In some embodiments, the composition comprises 40-50 μg (e.g., about 45 μg) of the one or more nucleic acid(s) (e.g. mRNA(s)). In some embodiments, the composition comprises 110-140 μg (e.g., about 120 μg) of the one or more nucleic acid(s) (e.g. mRNA(s)). In some embodiments, the composition comprises 200-250 μg (e.g., about 225 μg) of the one or more nucleic acid(s) (e.g. mRNA(s)).

The composition may comprise three nucleic acids (e.g., three mRNAs) as described herein encoding different polypeptides as described herein and the nucleic acids may be present in a weight ratio of 1:1:1. In some embodiments, the composition comprises a total of about 45 μg, about 120 μg or about 225 μg of the nucleic acids. The composition may therefore comprise about 15 μg, about 40 μg or about 75 μg of each of the three nucleic acids.

The composition may comprise two nucleic acids (e.g., two mRNAs) as described herein encoding different polypeptides as described herein and the nucleic acids may be present in a weight ratio of 1:1.

In further embodiments, a composition of the invention may be provided as a multi-valent single dose contains multiple (e.g., 2, 3, or 4) kinds of LNPs, each for a different antigen, and each kind of LNP has an mRNA amount of, e.g., 2.5 μg, about 5 μg, about 7.5 μg, about 10 μg, about 12.5 μg, about 15 μg, about 30 μg, about 45 μg, about 60 μg, about 75 μg, about 90 μg, about 105 μg, about 120 μg, about 135 μg, about 150 μg, about 165 μg, about 180 μg, about 195 μg, about 210 μg, about 225 μg, about 250 μg, about 275 μg, or about 300 μg.

In some embodiments, the subject is administered one or more nucleic acid compositions of the present invention. The nucleic acid compositions may comprise a nucleic acid comprising a nucleotide sequence encoding a polypeptide antigen as described herein. The nucleic acid compositions may be administered simultaneously, separately or sequentially. In some embodiments, the subject is administered a nucleic acid combination of the present invention. The nucleic acid combinations include combinations of two or more (e.g., three) nucleic acids as described herein. The nucleic acids within a combination may be administered simultaneously, separately or sequentially.

In some embodiments, the subject is administered one or more polypeptide compositions of the present invention. The polypeptide compositions may comprise a polypeptide antigen as described herein. The polypeptide compositions may be administered simultaneously, separately or sequentially. In some embodiments, the subject is administered a polypeptide combination of the present invention. The polypeptide combinations include combinations of two or more (e.g., three) polypeptides as described herein. The nucleic acids within a combination may be administered simultaneously, separately or sequentially.

In some embodiments, the subject is administered one or more nucleic acid compositions of the present invention and one or more polypeptide compositions of the invention. The nucleic acid composition and the one or more polypeptide compositions may be administered simultaneously, separately or sequentially.

Compositions administered separately or sequentially may be administered within 12 months of each other, within six months of each other, or within one month or less of each other (e.g. within 10 days). Compositions may be administered within 7 days, within 3 days, within 2 days, or within 24 hours of each other. Simultaneous administration may involve administering the compositions of the invention at the same time. Simultaneous administration may include administration of the compositions of the invention to a patient within 12 hours of each other, within 6 hours, within 3 hours, within 2 hours or within 1 hour of each other, typically within the same visit to a clinical centre.

The present invention also provides a kit comprising one or more compositions described herein in one or more containers or provides one or more composition as described herein in one or more containers and a physiological buffer for reconstitution in another container. The container(s) may contain a single-use dosage or multi-use dosage. The containers may be pre-treated glass vials or ampules. The kit may include instructions for use.

### Definitions

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

As used herein, the term “effective amount” refers to an amount (e.g., of a nucleic acid, a polypeptide, a combination or a composition as described herein) sufficient to effect beneficial or desired results. An effective amount can be administered in one or more administrations, applications or dosages, and is not intended to be limited to a particular formulation or administration route. The term “effective amount” includes, e.g., therapeutically effective amount and/or prophylactically effective amount. The term “effective amount” as used herein refers to an amount (e.g., of a nucleic acid, a polypeptide, a combination or a composition as described herein) which is effective for producing some desired therapeutic or prophylactic effects in the treatment or prevention of an infection, disease, disorder and/or condition at a reasonable benefit/risk ratio applicable to any medical treatment.

The term “fragment” or “variant” when referring to the polypeptides of the present disclosure include any polypeptides which retain at least some of the properties (e.g., specific antigenic property of the polypeptide or the ability of polypeptide to contribute to the induction of antibody binding) of the reference polypeptide. Fragments of polypeptides include N-terminally and/or C-terminally truncated fragments, e.g. C-terminal fragments and N-terminal fragments, as well as deletion fragments but do not include the naturally occurring full-length polypeptide (or mature polypeptide). A deletion fragment refers to a polypeptide with one or more internal amino acids deleted from the full-length polypeptide. Variants of polypeptides include fragments as described above, and also polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions. Such variations (i.e. truncations and/or amino acid substitutions, deletions, or insertions) may occur either on the amino acid level or correspondingly on the nucleic acid level.

Identity with respect to a sequence is defined herein as the percentage of nucleic acid or amino acid residues in the candidate sequence that are identical with the reference amino acid sequence after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides or the nucleic acids of two polynucleotides. For example, using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 [a standard scoring matrix; see Dayhoff et al., in Atlas of Protein Sequence and Structure, vol. 5, supp. 3 (1978)] can be used in conjunction with the computer program. The percent identity can be calculated as: the total number of identical matches multiplied by 100 and then divided by the sum of the length of the longer sequence within the matched span and the number of gaps introduced into the shorter sequences in order to align the two sequences.

As used herein, the term “kit” refers to a packaged set of related components, such as one or more compounds or compositions and one or more related materials such as solvents, solutions, buffers, instructions, or desiccants.

The term “linked” or “attached” as used herein refers to a first amino acid sequence or nucleotide sequence covalently joined to a second amino acid sequence or nucleotide sequence, respectively (e.g., a secretion signal peptide amino acid sequence and/or a heterologous transmembrane domain amino acid sequence linked to a C. acnes polypeptide amino acid sequence). The first amino acid or nucleotide sequence can be directly joined to the second amino acid or nucleotide sequence or alternatively an intervening sequence can covalently join the first sequence to the second sequence. The term “linked” means not only a fusion of a first amino acid sequence to a second amino acid sequence at the C-terminus or the N-terminus, but also includes insertion of the whole first amino acid sequence (or the second amino acid sequence) into any two amino acids in the second amino acid sequence (or the first amino acid sequence, respectively). In one embodiment, the first amino acid sequence can be linked to a second amino acid sequence by a peptide bond or a linker. The first nucleotide sequence can be linked to a second nucleotide sequence by a phosphodiester bond or a linker. The linker can be a peptide or a polypeptide (for polypeptide chains) or a nucleotide or a nucleotide chain (for nucleotide chains) or any chemical moiety (for both polypeptide and polynucleotide chains). The term “linked” is also indicated by a hyphen (-).

Acne lesions may be noninflammatory or inflammatory. Noninflammatory lesions of acne include open (blackheads) or closed (whiteheads) comedones. These lesions, especially closed comedones, may be precursors to the larger inflammatory lesions and therefore are of clinical importance. Inflammatory lesions may include papules, pustules, nodules and nodulocystic lesions, depending on the severity and location of the inflammation within the dermis. The papules and pustules may have surrounding halos of erythema allowing for their characterisation as inflammatory. Typically, nodules are erythematous and often tender and/or painful. In some embodiment, nodules are deep-seated in the skin (e.g. centred in the dermis and/or subcutis). Nodules may be greater than 5 mm in diameter.

Acne may be mild, moderate or severe, based on number and type of lesions affecting a specific skin area. Acne severity may be determined using the Investigator's Global Assessment (IGA) scale, which grades acne severity from 0 to 4. The IGA scale includes the following categories: clear (grade 0), almost clear (grade 1), mild severity (grade 2), moderate severity (grade 3), and severe (grade 4).

As used herein, the term “mild acne” refers to a severity grade of acne wherein some non-inflammatory lesions are present, with few inflammatory lesions. Typically, the subject has papules and/or pustules, e.g. the subject may have papules and/or pustules only. Typically, the subject does not have any nodulocystic lesions. Mild acne may be Grade 2 acne according to the IGA scale.

As used herein, the term “moderate acne” refers to a severity grade of acne wherein a subject has many comedones, papules and/or pustules. The subject may have a nodule, e.g. the subject may have no more than one nodule. Moderate acne typically affects more than half of a subject's face. Moderate acne may be Grade 3 acne according to the IGA scale.

As used herein, the term “severe acne” refers to a severity grade of acne wherein a subject has numerous comedones, papules and pustules. Typically the subject has one or more nodules and/or cysts. Severe acne typically affects a subject's entire face. For example, a subject's entire face may be covered with comedones, papules and pustules. Severe acne may be Grade 4 acne according to the IGA scale.

### EMBODIMENTS

The invention includes at least the following numbered embodiments:

1. A nucleic acid comprising a nucleotide sequence encoding a modified C. acnes CAMP2 polypeptide, wherein the modified C. acnes CAMP2 polypeptide comprises an amino acid sequence comprising a C. acnes CAMP2 polypeptide sequence and a transmembrane domain sequence.

2. The nucleic acid of embodiment 1, wherein the transmembrane domain sequence is positioned at the N terminus or the C terminus (e.g. the C terminus) of the modified C. acnes CAMP2 polypeptide.

3. The nucleic acid of embodiment 1 or 2, wherein the transmembrane domain sequence comprises a viral transmembrane domain sequence, which is optionally selected from the group consisting of: an influenza hemagglutinin (HA) transmembrane domain sequence, a SARS CoV-2 spike transmembrane domain sequence, a VZV gB transmembrane domain sequence, a VZV gE transmembrane domain sequence, a VZV gI transmembrane domain sequence, a VZV gK transmembrane domain sequence, a measles F-protein transmembrane domain sequence, a rubella E1 protein transmembrane domain sequence, a rubella E2 protein transmembrane domain sequence, a mumps F-protein transmembrane domain sequence, an Ebola GP protein transmembrane domain sequence and a rabies transmembrane domain sequence.

4. The nucleic acid of any one of embodiments 1-3, wherein the transmembrane domain sequence comprises an amino acid sequence according to any one of the sequences in Table 4.

5. The nucleic acid of any one of embodiments 1-4, wherein the transmembrane domain sequence comprises an amino acid sequence according to any one of SEQ ID NO: 208-209 (e.g., SEQ ID NO: 208).

6. The nucleic acid of any one of embodiments 1-5, wherein the modified C. acnes CAMP2 polypeptide comprises a non-native (e.g. viral) secretion signal peptide sequence.

7. The nucleic acid of embodiment 6, wherein the secretion signal peptide sequence is positioned at the N terminus or the C terminus (e.g. the N terminus) of the modified C. acnes CAMP2 polypeptide.

8. The nucleic acid of embodiment 6 or 7, wherein the secretion signal peptide sequence is a viral secretion signal peptide sequence, which optionally comprises a sequence selected from the group consisting of: an influenza hemagglutinin (HA) secretion signal peptide sequence, a SARS CoV-2 spike secretion signal peptide sequence, a VZV gB secretion signal peptide sequence, a VZV gE secretion signal peptide sequence, a VZV gI secretion signal peptide sequence, a VZV gK secretion signal peptide sequence, a measles F-protein secretion signal peptide sequence, a rubella E1 protein secretion signal peptide sequence, a rubella E2 protein secretion signal peptide sequence, a mumps F-protein secretion signal peptide sequence, an Ebola GP protein secretion signal peptide sequence, and a smallpox 6 kDa IC protein secretion signal peptide sequence.

9. The nucleic acid of any one of embodiments 6-8, wherein the secretion signal peptide sequence comprises a sequence according to any one of the sequences in Table 2 or 3.

10. The nucleic acid of any one of embodiments 6-9, wherein the secretion signal peptide sequence comprises a sequence according to any one of SEQ ID NO: 210-211 (e.g., SEQ ID NO: 210).

11. The nucleic acid of any one of embodiments 1-10, wherein the modified C. acnes CAMP2 polypeptide comprises a mutation at one or more (e.g., all) positions corresponding to an N-glycosylation site and/or one or more (e.g., all) positions corresponding to an O-glycosylation site in a native C. acnes CAMP2 polypeptide, optionally wherein the mutation is a single amino acid substitution.

12. The nucleic acid of embodiment 11, wherein:
(i) the C. acnes CAMP2 polypeptide comprises a mutation at position N166 relative to SEQ ID NO: 203, optionally wherein the mutation is a single amino acid substitution, e.g. N166S; and/or
(ii) the C. acnes CAMP2 polypeptide comprises a substitution of one or more serine (Ser) and/or threonine (Thr) residue(s).

13. The nucleic acid of any one of embodiments 1-12, wherein the nucleic acid is a messenger RNA (mRNA).

14. The nucleic acid of embodiment 13, wherein the mRNA comprises a 5′ cap, at least one 5′ untranslated region (5′ UTR), at least one 3′ untranslated region (3′ UTR), and/or at least one polyadenylation (poly(A)) sequence.

15. The nucleic acid of embodiments 13 or 14, wherein the mRNA is unmodified or:
(i) the mRNA comprises at least one chemical modification, optionally wherein the chemical modification is selected from the group consisting of e.g., 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, phosphoramidates, phosphorothioates, peptide nucleotides, methylphosphonates, 7-deazaguanosine, 5-methylcytosine, and inosine (e.g., wherein the chemical modification comprises N1-methylpseudouridine); and/or
(ii) the mRNA is synthesized from modified nucleotide analogues or derivatives of purines and pyrimidines, optionally wherein the modified nucleotide analogues or derivatives of purines and pyrimidines are selected from the group consisting of: 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, phosphoramidates, phosphorothioates, peptide nucleotides, methylphosphonates, 7-deazaguanosine, 5-methylcytosine, and inosine.

16. The nucleic acid of embodiment 15, wherein the mRNA comprises at least one chemical modification (e.g., wherein at least 20%, at least 30%, at least 40%, at least 50%, at least 60%, at least 70%, at least 80%, at least 85%, at least 90%, at least 95%, or 100% of the uridines in the mRNA are chemically modified), optionally wherein the chemical modification comprises N1-methylpseudouridine e.g. in place of every uridine.

17. The nucleic acid of any one of embodiments 13-16, wherein the mRNA is a self-replicating mRNA or a non-replicating mRNA.

18. The nucleic acid of embodiment 17, wherein the mRNA is a non-replicating mRNA.

19. The nucleic acid of any one of embodiments 1-18, wherein:
(i) the modified C. acnes CAMP2 polypeptide comprises a sequence according to any one of SEQ ID NO: 203, SEQ ID NO: 207, SEQ ID NO: 43-58, SEQ ID NO: 1-16 or SEQ ID NO: 339-363 (e.g. SEQ ID NO: 207), or a sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g. at least 85%, 90% or 95%) identity thereto; and/or
(ii) the C. acnes CAMP2 polypeptide comprises an amino acid substitution at one or more positions corresponding to residues 6, 9, 11, 18, 19, 21, 24, 29, 30, 37, 48, 61, 65, 68, 76, 87, 91, 92, 98, 100, 106, 118, 128, 138, 143, 145, 154, 169, 177, 179, 189, 207, 221 and/or 223 of SEQ ID NO: 203, e.g, wherein the C. acnes CAMP2 polypeptide comprises an amino acid substitution at one or more positions corresponding to residues T6 (e.g. T6I), A9 (e.g. A9T), S11 (e.g. S11A), S18 (e.g. S18N), D19 (e.g. D19E or D19Y), R21 (e.g. R21H), I24 (e.g. I24M, I24L or I24T), A29 (e.g. A29P), H30 (e.g. H30R), V37 (e.g. V37A), D48 (e.g. D48N), R61 (e.g. R61H), E65 (e.g. E65D), A68 (e.g. A68T), D76 (e.g. D76N), V87 (e.g. V87A), 191 (e.g. 191V), D92 (e.g. D92G), T98 (e.g. T98K), T100 (e.g. T1001), R106 (e.g. R106S), K118 (e.g. K118N), S128 (e.g. S128T), A138 (e.g. A138T), R143 (e.g. R143H), E145 (e.g. E145D or E145K), T154 (e.g. T154A), K169 (e.g. K169R), N177 (e.g. N177D), D179 (e.g. D179N or D179H), A189 (e.g. A189E), N207 (e.g. N207D or N207A), E221 (e.g. E221K) and/or L223 (e.g. L223F) of SEQ ID NO: 203.

20. The nucleic acid of any one of embodiments 1-19, wherein the nucleic acid comprises a nucleotide sequence according to any one of SEQ ID NO: 153-167, SEQ ID NO: 87-101, SEQ ID NO: 391-392 or SEQ ID NO: 398-399 (e.g., SEQ ID NO: 91, SEQ ID NO: 90, SEQ ID NO: 391 or SEQ ID NO: 392, such as SEQ ID NO: 91), or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g. at least 75%, 80% or 85%, such as 75% or 80%) identity thereto.

21. A modified C. acnes CAMP2 polypeptide having an amino acid sequence comprising a C. acnes CAMP2 polypeptide sequence and a transmembrane domain sequence.

22. The modified C. acnes CAMP2 polypeptide of embodiment 21, wherein the transmembrane domain sequence is positioned at the N terminus or the C-terminus (e.g. the C terminus) of the modified C. acnes CAMP2 polypeptide.

23. The modified C. acnes CAMP2 polypeptide of embodiment 21 or 22, wherein the transmembrane domain sequence comprises a viral transmembrane domain sequence, which is optionally selected from the group consisting of: an influenza hemagglutinin (HA) transmembrane domain sequence, a SARS CoV-2 spike transmembrane domain sequence, a VZV gB transmembrane domain sequence, a VZV gE transmembrane domain sequence, a VZV gI transmembrane domain sequence, a VZV gK transmembrane domain sequence, a measles F-protein transmembrane domain sequence, a rubella E1 protein transmembrane domain sequence, a rubella E2 protein transmembrane domain sequence, a mumps F-protein transmembrane domain sequence, an Ebola GP protein transmembrane domain sequence and a rabies transmembrane domain sequence.

24. The modified C. acnes CAMP2 polypeptide of any one of embodiments 21-23, wherein the transmembrane domain sequence comprises an amino acid sequence according to any one of the sequences in Table 4.

25. The modified C. acnes CAMP2 polypeptide of any one of embodiments 21-24, wherein the transmembrane domain sequence comprises an amino acid sequence according to any one of SEQ ID NO: 208-209.

26. The modified C. acnes CAMP2 polypeptide of any one of embodiments 21-25, wherein the modified C. acnes CAMP2 polypeptide comprises a mutation at one or more (e.g., all) positions corresponding to an N-glycosylation site and/or one or more (e.g., all) positions corresponding to an O-glycosylation site in a native C. acnes CAMP2 polypeptide, optionally wherein the mutation is a single amino acid substitution.

27. The modified C. acnes CAMP2 polypeptide of embodiment 26, wherein the C. acnes CAMP2 polypeptide comprises a mutation at position N166 relative to SEQ ID NO: 203, optionally wherein:
(i) the mutation is a single amino acid substitution, e.g. N166S; and/or
(ii) the C. acnes CAMP2 polypeptide comprises a substitution of one or more serine (Ser) and/or threonine (Thr) residue(s).

28. The modified C. acnes CAMP2 polypeptide of any one of embodiments 21-27, wherein
(i) the modified C. acnes CAMP2 polypeptide comprises a sequence according to any one of SEQ ID NO: 203, SEQ ID NO: 207, SEQ ID NO: 43-58, SEQ ID NO: 1-16 or SEQ ID NO: 339-363 (e.g. SEQ ID NO: 207), or a sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g. at least 85%, 90% or 95%) identity thereto; and/or
(ii) the C. acnes CAMP2 polypeptide comprises an amino acid substitution at one or more positions corresponding to residues 6, 9, 11, 18, 19, 21, 24, 29, 30, 37, 48, 61, 65, 68, 76, 87, 91, 92, 98, 100, 106, 118, 128, 138, 143, 145, 154, 169, 177, 179, 189, 207, 221 and/or 223 of SEQ ID NO: 203, e.g, wherein the C. acnes CAMP2 polypeptide comprises an amino acid substitution at one or more positions corresponding to residues T6 (e.g. T6I), A9 (e.g. A9T), S11 (e.g. S11A), S18 (e.g. S18N), D19 (e.g. D19E or D19Y), R21 (e.g. R21H), I24 (e.g. I24M, I24L or I24T), A29 (e.g. A29P), H30 (e.g. H30R), V37 (e.g. V37A), D48 (e.g. D48N), R61 (e.g. R61H), E65 (e.g. E65D), A68 (e.g. A68T), D76 (e.g. D76N), V87 (e.g. V87A), 191 (e.g. 191V), D92 (e.g. D92G), T98 (e.g. T98K), T100 (e.g. T1001), R106 (e.g. R106S), K118 (e.g. K118N), S128 (e.g. S128T), A138 (e.g. A138T), R143 (e.g. R143H), E145 (e.g. E145D or E145K), T154 (e.g. T154A), K169 (e.g. K169R), N177 (e.g. N177D), D179 (e.g. D179N or D179H), A189 (e.g. A189E), N207 (e.g. N207D or N207A), E221 (e.g. E221K) and/or L223 (e.g. L223F) of SEQ ID NO: 203.

29. A composition, optionally an immunogenic composition, comprising the nucleic acid (e.g. a mRNA) of any one of embodiments 1-20.

30. An immunogenic composition comprising a nucleic acid comprising a nucleotide sequence encoding a C. acnes CAMP2 polypeptide, wherein the nucleic acid is a mRNA.

31. A composition, optionally an immunogenic composition, comprising a nucleic acid comprising a nucleotide sequence encoding a C. acnes CAMP2 polypeptide and a lipid nanoparticle (LNP), optionally wherein the nucleotide sequence encoding the C. acnes CAMP2 polypeptide is encapsulated in the LNP.

32. A composition, optionally an immunogenic composition, comprising:
(a) a nucleic acid (e.g., a mRNA) comprising a nucleotide sequence encoding a C. acnes CAMP2 polypeptide; and
(b) one or more of:
   (i) a nucleic acid comprising a nucleotide sequence encoding a C. acnes DsA1 polypeptide;
   (ii) a nucleic acid comprising a nucleotide sequence encoding a C. acnes DsA2 polypeptide;
   (iii) a nucleic acid comprising a nucleotide sequence encoding a C. acnes PITP polypeptide;
   (iv) a nucleic acid comprising a nucleotide sequence encoding a chimeric C. acnes DsA1/DsA2 polypeptide; and
   (v) a nucleic acid comprising a nucleotide sequence encoding a chimeric C. acnes DsA1/DsA2/PITP polypeptide.

33. The composition of embodiment 32, wherein the composition comprises:
the nucleic acid in (a) and the nucleic acid in (b) (i), and optionally the nucleic acid in (b) (ii) and/or the nucleic acid in (b) (iii);
the nucleic acid in (a) and the nucleic acid in (b) (ii), and optionally the nucleic acid in (b) (i) and/or the nucleic acid in (b) (iii);
the nucleic acid in (a) and the nucleic acid in (b) (iii), and optionally the nucleic acid in (b) (i) and/or the nucleic acid in (b) (ii);
the nucleic acid in (a) and the nucleic acid in (b) (iv), and optionally the nucleic acid in (b) (iii);
the nucleic acid in (a) and the nucleic acid in (b) (v); or the nucleic acid in (a) and the nucleic acid in (b) (i)-(iii).

34. The composition of embodiment 32 or 33, wherein the composition comprises the nucleic acid in (a), the nucleic acid in (b) (iii) and the nucleic acid in (b) (iv), optionally wherein the nucleic acids are present in a weight ratio of 1:1:1.

35. The composition of embodiment 34, wherein
the C. acnes CAMP2 polypeptide comprises a sequence according to SEQ ID NO: 203 or a sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g. at least 85%, 90% or 95%) identity thereto;
the chimeric C. acnes DsA1/DsA2 polypeptide comprises a sequence according to SEQ ID NO: 70 or a sequence having at least 90%, at least 91%, at least 92%, at least 93%, at least 94%, at least 95%, at least 96%, at least 97%, at least 98%, or at least 99% (e.g., at least 90%) identity thereto; and
the C. acnes PITP polypeptide comprises a sequence according to SEQ ID NO: 73 or a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g., at least 75%) identity thereto.

36. The composition of embodiment 32 or 33, wherein the composition comprises the nucleic acid in (a) and the nucleic acid in (b) (v), optionally wherein the nucleic acid in b (v) has a nucleotide sequence according to SEQ ID NO: 80, SEQ ID NO: 82 or SEQ ID NO: 83 (e.g. SEQ ID NO: 80 or SEQ ID NO: 83).

37. The composition of any one of embodiments 32-35, wherein any two or more nucleic acids in (a) and (b) (i)-(v) are located on the same nucleic acid molecule or on different nucleic acid molecules (e.g, wherein all the nucleic acids in the composition are on the same nucleic acid molecule or wherein all the nucleic acids in the composition are on individual nucleic acid molecules).

38. The composition of embodiment 34, wherein the nucleic acid in (a), the nucleic acid in (b) (iii) and the nucleic acid in (b) (iv) are located on the same nucleic acid molecule or separate nucleic acid molecules.

39. The composition of embodiment 35, wherein the nucleic acid in (a) and the nucleic acid in (b) (v) are located on the same nucleic acid molecule or separate nucleic acid molecules.

40. The composition of any one of embodiments 29-31, wherein the composition further comprises one or more of:
(i) a nucleic acid comprising a nucleotide sequence encoding a C. acnes DsA1 polypeptide;
(ii) a nucleic acid comprising a nucleotide sequence encoding a C. acnes DsA2 polypeptide;
(iii) a nucleic acid comprising a nucleotide sequence encoding a C. acnes PITP polypeptide;
(iv) a nucleic acid comprising a nucleotide sequence encoding a chimeric C. acnes DsA1/DsA2 polypeptide; and
(v) a nucleic acid comprising a nucleotide sequence encoding a chimeric C. acnes DsA1/DsA2/PITP polypeptide.

41. The composition of embodiment 40, wherein the composition comprises:
the nucleic acid in (i), and optionally the nucleic acid in (ii) and/or the nucleic acid in (iii);
the nucleic acid in (ii), and optionally the nucleic acid in (i) and/or the nucleic acid in (iii);
the nucleic acid in (iii), and optionally the nucleic acid in (i) and/or the nucleic acid in (ii);
the nucleic acid in (iv), and optionally the nucleic acid in (iii);
the nucleic acid in (v); or
the nucleic acid in (i)-(iii).

42. The composition of embodiment 40 or 41, wherein the composition comprises the nucleic acid in (iii) and the nucleic acid in (iv), optionally wherein the nucleic acids are present in a weight ratio of 1:1:1.

43. The composition of embodiment 42, wherein:
(i) the C. acnes CAMP2 polypeptide comprises a sequence according to SEQ ID NO: 207 or a sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% (e.g. at least 60%, such as at least 85%, 90% or 95%) identity thereto; the chimeric C. acnes DsA1/DsA2 polypeptide comprises a sequence according to SEQ ID NO: 70 or a sequence having at least 90%, at least 91%, at least 92%, at least 93%, at least 94%, at least 95%, at least 96%, at least 97%, at least 98%, or at least 99% (e.g., at least 90%) identity thereto; and the C. acnes PITP polypeptide comprises a sequence according to SEQ ID NO: 73 or a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g., at least 75%) identity thereto; or
(ii) the nucleotide sequence encoding the C. acnes CAMP2 polypeptide comprises a sequence according to SEQ ID NO: 91 or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g., at least 75%, 80% or 85%) identity thereto;
   the nucleotide sequence encoding the chimeric C. acnes DsA1/DsA2 polypeptide comprises a sequence according to SEQ ID NO: 113 or a sequence having at least 75%, at least 80%, at least 85%, at least 90%, at least 91%, at least 92%, at least 93%, at least 94%, at least 95%, at least 96%, at least 97%, at least 98%, or at least 99% (e.g., at least 75%) identity thereto; and
   the nucleotide sequence encoding the C. acnes PITP polypeptide comprises a sequence according to SEQ ID NO: 115 or a sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% (e.g., at least 50%) identity thereto.

44. The composition of embodiment 40 or 41, wherein the composition comprises the nucleic acid in (v), optionally wherein the nucleic acid in (v) comprises a nucleotide sequence according to SEQ ID NO: 80, SEQ ID NO: 82 or SEQ ID NO: 83 (e.g., SEQ ID NO: 80 or 83).

45. The composition of any one of embodiments 40-44, wherein any two or more of (a) the nucleic acid of any one of embodiments 1-20 and (b) the nucleic acids in (i)-(v) are located on the same nucleic acid molecule or on different nucleic acid molecules (e.g. on the same nucleic acid molecule).

46. The composition of embodiment 45, wherein the nucleic acid in (a), the nucleic acid in (b) (iii) and the nucleic acid in (b) (iv) are located on the same nucleic acid molecule.

47. The composition of any one of embodiments 40-44, wherein any two or more of (a) the nucleic acid comprising a nucleotide sequence encoding a C. acnes CAMP2 polypeptide and (b) the nucleic acids in (i)-(v) are located on the same nucleic acid molecule or on different nucleic acid molecules (e.g. on the same nucleic acid molecule).

48. The composition of embodiment 47, wherein the nucleic acid in (a) and the nucleic acid in (b) (v) are located on the same nucleic acid molecule.

49. The composition of any one of embodiments 29-30 or 32-48, wherein the composition further comprises a lipid nanoparticle (LNP), optionally wherein any one or more nucleic acids are encapsulated in the LNP, e.g, wherein the LNP has an average diameter of 30 nm to 200 nm (e.g. an average diameter of 80 nm to 150 nm).

50. The composition of embodiment 31 or 49, wherein the LNP comprises at least one cationic lipid, optionally wherein the cationic lipid:
is biodegradable or is not biodegradable, and/or
the cationic lipid is cleavable or is not cleavable.

51. The composition of embodiment 49 or 50, wherein the cationic lipid is selected from the group consisting of OF-02, cKK-E10, OF-Deg-Lin, GL-HEPES-E3-E10-DS-3-E18-1, GL-HEPES-E3-E12-DS-4-E10, GL-HEPES-E3-E12-DS-3-E14, SM-102, ALC-0315, IM-001 and IS-001.

52. The composition of any one of embodiments 49-51, wherein the cationic lipid is selected from the group consisting of OF-02, cKK-E10, GL-HEPES-E3-E12-DS-4-E10, IM-001 and IS-001 (e.g., wherein the cationic lipid is GL-HEPES-E3-E12-DS-4-E10).

53. The composition of any one of embodiments 31 or 49-52, wherein the LNP further comprises a polyethylene glycol (PEG) conjugated (PEGylated) lipid, a cholesterol-based lipid, and a helper lipid, optionally wherein the LNP comprises: a cationic lipid at a molar ratio of 35% to 55%; a polyethylene glycol (PEG) conjugated (PEGylated) lipid at a molar ratio of 0.25% to 2.75%; a cholesterol-based lipid at a molar ratio of 20% to 45%; and a helper lipid at a molar ratio of 5% to 35%, wherein all of the molar ratios are relative to the total lipid content of the LNP, e.g, wherein the LNP comprises a cationic lipid at a molar ratio of 40%, a PEGylated lipid at a molar ratio of 1.5%, a cholesterol-based lipid at a molar ratio of 28.5%, and a helper lipid at a molar ratio of 30%.

54. The composition of any one of embodiments 49-53, wherein any two or more (e.g., three) nucleic acids are co-encapsulated in a single LNP.

55. The composition of any one of embodiments 49-53, wherein any two or more (e.g., three) nucleic acids are encapsulated in separate LNPs.

56. The composition of any one of embodiments 29-55, wherein the mRNA comprises a 5′ cap, at least one 5′ untranslated region (5′ UTR), at least one 3′ untranslated region (3′ UTR), and/or at least one polyadenylation (poly(A)) sequence.

57. The composition of any one of embodiments 29-56, wherein the mRNA is unmodified or:
(i) the mRNA comprises at least one chemical modification, optionally wherein the chemical modification is selected from the group consisting of e.g., 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, phosphoramidates, phosphorothioates, peptide nucleotides, methylphosphonates, 7-deazaguanosine, 5-methylcytosine, and inosine (e.g., wherein the chemical modification comprises N1-methylpseudouridine); and/or
(ii) the mRNA is synthesized from modified nucleotide analogues or derivatives of purines and pyrimidines, optionally wherein the modified nucleotide analogues or derivatives of purines and pyrimidines are selected from the group consisting of:
   1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, phosphoramidates, phosphorothioates, peptide nucleotides, methylphosphonates, 7-deazaguanosine, 5-methylcytosine, and inosine.

58. The composition of any one of embodiments 29-57, wherein the mRNA comprises at least one chemical modification (e.g., wherein at least 20%, at least 30%, at least 40%, at least 50%, at least 60%, at least 70%, at least 80%, at least 85%, at least 90%, at least 95%, or 100% of the uridines in the mRNA are chemically modified), optionally wherein the chemical modification comprises N1-methylpseudouridine e.g. in place of every uridine.

59. The composition of any one of embodiments 29-58, wherein the mRNA is a self-replicating mRNA or a non-replicating mRNA, e.g, wherein the mRNA is a non-replicating mRNA.

60. The composition of any one of embodiments 29-59, wherein the composition can be in a frozen liquid form or in a lyophilized form (e.g., in a lyophilized form).

61. A composition, optionally an immunogenic composition, comprising the modified C. acnes CAMP2 polypeptide as defined in any one of embodiments 21-28.

62. The composition of embodiment 61, wherein the composition further comprises a C. acnes DsA1 polypeptide.

63. The composition of embodiment 61 or 62, wherein the composition further comprises a C. acnes DsA2 polypeptide.

64. The composition of any of embodiments 61-63, wherein the composition further comprises a C. acnes PITP polypeptide.

65. The composition of any of embodiments 61-64, wherein the composition further comprises a chimeric C. acnes DsA1/DsA2 polypeptide.

66. The composition of any of embodiments 61-65, wherein the composition further comprises a chimeric C. acnes DsA1/DsA2/PITP polypeptide.

67. The composition of embodiment 61, wherein the composition further comprises a chimeric C. acnes DsA1/DsA2 polypeptide and a C. acnes PITP polypeptide.

68. A composition, optionally an immunogenic composition, comprising:
(a) a C. acnes CAMP2 polypeptide; and
(b) one or more of:
   (i) a C. acnes DsA1 polypeptide,
   (ii) a C. acnes DsA2 polypeptide,
   (iii) a C. acnes PITP polypeptide,
   (iv) a chimeric C. acnes DsA1/DsA2 polypeptide, and
   (v) a chimeric C. acnes DsA1/DsA2/PITP polypeptide.

69. The composition of embodiment 68, wherein the composition comprises:
the polypeptide in (a) and the polypeptide in (b) (i), and optionally the polypeptide in (b) (ii) and/or the polypeptide in (b) (iii);
the polypeptide in (a) and the polypeptide in (b) (ii), and optionally the polypeptide in (b) (i) and/or the polypeptide in (b) (iii);
the polypeptide in (a) and the polypeptide in (b) (iii), and optionally the polypeptide in (b) (i) and/or the polypeptide in (b) (ii);
the polypeptide in (a) and the polypeptide in (b) (iv), and optionally the polypeptide in (b) (iii);
the polypeptide in (a) and the polypeptide in (b) (v); or
the polypeptide in (a) and the polypeptide in (b) (i)-(iii).

70. The composition of any one of embodiments 61-69, wherein the composition further comprises an adjuvant, optionally wherein the adjuvant is selected from the group consisting of: Aluminum based adjuvant (e.g. AlOOH), Squalene based oil in water emulsion adjuvants (e.g. AF03, AS03, MF59) and Liposome-based adjuvants comprising a saponin and a TLR4 agonist (e.g. SPA14, AS01, LEQ), further optionally wherein the adjuvant is selected from the group consisting of AlOOH, AF03 and SPA14.

71. The composition of any one of embodiments 29-70, wherein one or more of the C. acnes CAMP2 polypeptide, the C. acnes DsA1 polypeptide, the C. acnes DsA2 polypeptide, the C. acnes PITP polypeptide, the chimeric C. acnes DsA1/DsA2 polypeptide, and the chimeric C. acnes DsA1/DsA2/PITP polypeptide further comprises:
(i) a non-native transmembrane domain sequence (e.g. a viral transmembrane domain sequence; and/or
(ii) a non-native secretion signal peptide sequence.

72. The composition of any one of embodiments 61-71, wherein the composition can be in a frozen liquid form or in a lyophilized form.

73. A combination comprising:
(a) a nucleic acid (e.g., a mRNA) comprising a nucleotide sequence encoding a C. acnes CAMP2 polypeptide; and
(b) one or more of:
   (i) a nucleic acid comprising a nucleotide sequence encoding a C. acnes DsA1 polypeptide;
   (ii) a nucleic acid comprising a nucleotide sequence encoding a C. acnes DsA2 polypeptide;
   (iii) a nucleic acid comprising a nucleotide sequence encoding a C. acnes PITP polypeptide;
   (iv) a nucleic acid comprising a nucleotide sequence encoding a chimeric C. acnes DsA1/DsA2 polypeptide; and
   (v) a nucleic acid comprising a nucleotide sequence encoding a chimeric C. acnes DsA1/DsA2/PITP polypeptide.

74. The combination of embodiment 73, wherein the combination comprises:
the nucleic acid in (a) and the nucleic acid in (b) (i), and optionally the nucleic acid in (b) (ii) and/or the nucleic acid in (b) (iii);
the nucleic acid in (a) and the nucleic acid in (b) (ii), and optionally the nucleic acid in (b) (i) and/or the nucleic acid in (b) (iii);
the nucleic acid in (a) and the nucleic acid in (b) (iii), and optionally the nucleic acid in (b) (i) and/or the nucleic acid in (b) (ii);
the nucleic acid in (a) and the nucleic acid in (b) (iv), and optionally the nucleic acid in (b) (iii);
the nucleic acid in (a) and the nucleic acid in (b) (v); or
the nucleic acid in (a) and the nucleic acid in (b) (i)-(iii).

75. The combination of embodiment 73 or 74, wherein the combination comprises the nucleic acid in (a), the nucleic acid in (b) (iii) and the nucleic acid in (b) (iv).

76. The combination of embodiment 73 or 74, wherein the combination comprises the nucleic acid in (a) and the nucleic acid in (b) (v).

77. The combination of any one of embodiments 73-76, wherein any two or more nucleic acids in (a) and (b) (i)-(v) are located on the same nucleic acid molecule or on different nucleic acid molecules (e.g, wherein all the nucleic acids in the composition are on the same nucleic acid molecule or wherein all the nucleic acids in the composition are on individual nucleic acid molecules).

78. The combination of embodiment 75, wherein the nucleic acid in (a), the nucleic acid in (b) (iii) and the nucleic acid in (b) (iv) are located on the same nucleic acid molecule or on individual nucleic acid molecules.

79. The combination of embodiment 76, wherein the nucleic acid in (a) and the nucleic acid in (b) (v) are located on the same nucleic acid molecule.

80. A combination comprising the nucleic acid (e.g. a mRNA) of any one of embodiments 1-20 and one or more of:
(i) a nucleic acid comprising a nucleotide sequence encoding a C. acnes DsA1 polypeptide;
(ii) a nucleic acid comprising a nucleotide sequence encoding a C. acnes DsA2 polypeptide;
(iii) a nucleic acid comprising a nucleotide sequence encoding a C. acnes PITP polypeptide;
(iv) a nucleic acid comprising a nucleotide sequence encoding a chimeric C. acnes DsA1/DsA2 polypeptide; and
(v) a nucleic acid comprising a nucleotide sequence encoding a chimeric C. acnes DsA1/DsA2/PITP polypeptide.

81. The combination of embodiment 80, wherein the combination comprises:
the nucleic acid in (i), and optionally the nucleic acid in (ii) and/or the nucleic acid in (iii);
the nucleic acid in (ii), and optionally the nucleic acid in (i) and/or the nucleic acid in (iii);
the nucleic acid in (iii), and optionally the nucleic acid in (i) and/or the nucleic acid in (ii);
the nucleic acid in (iv), and optionally the nucleic acid in (iii);
the nucleic acid in (v); or
the nucleic acid in (i)-(iii).

82. The combination of embodiment 80 or 81, wherein the combination comprises the nucleic acid in (iii) and the nucleic acid in (iv).

83. The combination of embodiment 80 or 81, wherein the combination comprises the nucleic acid in (v)

84. The combination of any one of embodiments 80-83, wherein any two or more of (a) the nucleic acid of any one of embodiments 1-20 and (b) the nucleic acids in (i)-(v) are located on the same nucleic acid molecule or on different nucleic acid molecules (e.g. on the same nucleic acid molecule).

85. The combination of embodiment 84, wherein the nucleic acid in (a), the nucleic acid in (b) (iii) and the nucleic acid in (b) (iv) are located on the same nucleic acid molecule or separate nucleic acid molecules.

86. The combination of embodiment 84, wherein the nucleic acid in (a) and the nucleic acid in (b) (v) are located on the same nucleic acid molecule or separate nucleic acid molecules.

87. A combination comprising the modified C. acnes CAMP2 polypeptide as defined in any one of embodiments 21-28 and one or more of:
(i) a C. acnes DsA1 polypeptide;
(ii) a C. acnes DsA2 polypeptide;
(iii) a C. acnes PITP polypeptide;
(iv) a chimeric C. acnes DsA1/DsA2 polypeptide; and
(v) a chimeric C. acnes DsA1/DsA2/PITP polypeptide.

88. The combination of embodiment 87, wherein the combination comprises:
the polypeptide in (i), and optionally the polypeptide in (ii) and/or the polypeptide in (iii);
the polypeptide in (ii), and optionally the polypeptide in (i) and/or the polypeptide in (iii);
the polypeptide in (iii), and optionally the polypeptide in (i) and/or the polypeptide in (ii);
the polypeptide in (iv), and optionally the polypeptide in (iii);
the polypeptide in (v); or
the polypeptide in (i)-(iii).

89. A combination, optionally an immunogenic combination, comprising:
(a) a C. acnes CAMP2 polypeptide; and
(b) one or more of:
   (i) a C. acnes DsA1 polypeptide,
   (ii) a C. acnes DsA2 polypeptide,
   (iii) a C. acnes PITP polypeptide,
   (iv) a chimeric C. acnes DsA1/DsA2 polypeptide, and
   (v) a chimeric C. acnes DsA1/DsA2/PITP polypeptide.

90. The combination of embodiment 89, wherein the combination comprises:
the polypeptide in (a) and the polypeptide in (b) (i), and optionally the polypeptide in (b) (ii) and/or the polypeptide in (b) (iii);
the polypeptide in (a) and the polypeptide in (b) (ii), and optionally the polypeptide in (b) (i) and/or the polypeptide in (b) (iii);
the polypeptide in (a) and the polypeptide in (b) (iii), and optionally the polypeptide in (b) (i) and/or the polypeptide in (b) (ii);
the polypeptide in (a) and the polypeptide in (b) (iv), and optionally the polypeptide in (b) (iii);
the polypeptide in (a) and the polypeptide in (b) (v); or
the polypeptide in (a) and the polypeptide in (b) (i)-(iii).

91. The combination of any one of embodiments 73-90, wherein the one or more of the C. acnes CAMP2 polypeptide, the C. acnes DsA1 polypeptide, the C. acnes DsA2 polypeptide, the C. acnes PITP polypeptide, the chimeric C. acnes DsA1/DsA2 polypeptide, and the chimeric C. acnes DsA1/DsA2/PITP polypeptide further comprise a non-native transmembrane domain sequence (e.g. a viral transmembrane domain sequence).

92. The combination of any one of embodiments 73-91, wherein the one or more of the C. acnes CAMP2 polypeptide, the C. acnes DsA1 polypeptide, the C. acnes DsA2 polypeptide, the C. acnes PITP polypeptide, the chimeric C. acnes DsA1/DsA2 polypeptide, and the chimeric C. acnes DsA1/DsA2/PITP polypeptide further comprise a non-native secretion signal peptide sequence.

93. The combination of any one of embodiments 73-92, wherein the nucleic acids are present in the same composition.

94. The combination of any one of embodiments 73-92, wherein the nucleic acids are present in two or more separate compositions.

95. The combination of any one of embodiments 89-92, wherein the polypeptides are present in the same composition.

96. The combination of any one of embodiments 89-92, wherein the polypeptides are present in two or more separate compositions.

97. A nucleic acid comprising a nucleotide sequence encoding a chimeric C. acnes DsA1/DsA2 polypeptide, wherein the chimeric C. acnes DsA1/DsA2 polypeptide comprises an amino acid sequence comprising:
(i) the sequence of SEQ ID NO: 70, or a sequence having at least 90%, at least 91%, at least 92%, at least 93%, at least 94%, at least 95%, at least 96%, at least 97%, at least 98%, or at least 99% (e.g., at least 90%) identity to SEQ ID NO: 70; and
(ii) a secretion signal peptide sequence e.g., a non-native secretion signal peptide sequence and/or a heterologous transmembrane domain,
optionally wherein the chimeric C. acnes DsA1/DsA2 polypeptide comprises a sequence according to SEQ ID NO: 28 or a sequence having at least 90%, at least 91%, at least 92%, at least 93%, at least 94%, at least 95%, at least 96%, at least 97%, at least 98%, or at least 99% (e.g., at least 90%) identity to SEQ ID NO: 28.

98. The nucleic acid of embodiment 97 wherein the chimeric C. acnes DsA1/DsA2 polypeptide comprises a non-native secretion signal peptide sequence.

99. The nucleic acid of embodiment 97 or 98, wherein the secretion signal peptide sequence is positioned at the N terminus or the C terminus (e.g. the N terminus) of the chimeric C. acnes DsA1/DsA2 polypeptide.

100. The nucleic acid of any one of embodiments 97-99, wherein the heterologous transmembrane domain is positioned at the N terminus or the C terminus (e.g. the C terminus) of the chimeric C. acnes DsA1/DsA2 polypeptide.

101. The nucleic acid of any one of embodiments 97-100, wherein the nucleic acid comprises:
(i) a sequence of SEQ ID NO: 113 or a sequence having at least 75%, at least 80%, at least 85%, at least 90%, at least 91%, at least 92%, at least 93%, at least 94%, at least 95%, at least 96%, at least 97%, at least 98%, or at least 99% (e.g., at least 75%) identity to SEQ ID NO: 113; or
(ii) a sequence of SEQ ID NO: 179 or a sequence having at least 75%, at least 80%, at least 85%, at least 90%, at least 91%, at least 92%, at least 93%, at least 94%, at least 95%, at least 96%, at least 97%, at least 98%, or at least 99% (e.g., at least 75%) identity to SEQ ID NO: 179.

102. The nucleic acid of any one of embodiments 97-101, wherein the nucleic acid is a mRNA.

103. A chimeric C. acnes DsA1/DsA2 polypeptide comprising (i) the sequence of SEQ ID NO: 70, or a sequence having at least 90%, at least 91%, at least 92%, at least 93%, at least 94%, at least 95%, at least 96%, at least 97%, at least 98%, or at least 99% (e.g., at least 90%) identity to SEQ ID NO: 70; and (ii) a heterologous transmembrane domain.

104. The chimeric C. acnes DsA1/DsA2 polypeptide of embodiment 103, wherein the heterologous transmembrane domain is positioned at the N terminus or the C terminus (e.g. the C terminus) of the chimeric C. acnes DsA1/DsA2 polypeptide.

105. The chimeric C. acnes DsA1/DsA2 polypeptide of any one of embodiments 103-104, wherein the polypeptide comprises the sequence of SEQ ID NO: 28 or a sequence having at least 90%, at least 91%, at least 92%, at least 93%, at least 94%, at least 95%, at least 96%, at least 97%, at least 98%, or at least 99% (e.g., at least 90%) identity to SEQ ID NO: 28.

106. A nucleic acid comprising a nucleotide sequence encoding a C. acnes PITP polypeptide, wherein the C. acnes PITP polypeptide comprises an amino acid sequence comprising:
(i) the sequence of SEQ ID NO: 73, or a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g., at least 75%) identity thereto; and
(ii) a secretion signal peptide sequence, e.g., a non-native secretion signal peptide sequence, and/or a heterologous transmembrane domain, optionally wherein the C. acnes PITP polypeptide comprises an amino acid sequence according to SEQ ID NO: 31 or an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g., at least 75%) identity to SEQ ID NO: 31.

107. The nucleic acid of embodiment 106, wherein the C. acnes PITP polypeptide comprises a non-native secretion signal peptide sequence.

108. The nucleic acid of embodiment 106 or 107, wherein the heterologous transmembrane domain is positioned at the at the N terminus or the C terminus (e.g. the C terminus) of the C. acnes PITP polypeptide.

109. The nucleic acid of any one of embodiments 106-108, wherein the secretion signal peptide sequence is positioned at the N terminus or the C terminus (e.g. the N terminus) of the C. acnes PITP polypeptide.

110. The nucleic acid of any one of embodiments 106-109, wherein the nucleic acid comprises a sequence of SEQ ID NO: 115 or a sequence of SEQ ID NO: 182 or a sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% (e.g., at least 75%) identity to SEQ ID NO: 115 or SEQ ID NO: 182.

111. The nucleic acid of any one of embodiments 106-110, wherein the nucleic acid is mRNA.

112. A C. acnes PITP polypeptide comprising (i) the sequence of SEQ ID NO: 73, or a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g., at least 75%) identity to SEQ ID NO: 73, and (ii) a heterologous transmembrane domain.

113. The C. acnes PITP polypeptide of embodiment 112, wherein the heterologous transmembrane domain is positioned at the N terminus or the C terminus (e.g. the C terminus) of the C. acnes PITP polypeptide.

114. The C. acnes PITP polypeptide of any one of embodiments 112-113, wherein the polypeptide comprises a sequence of SEQ ID NO: 31 or a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g., at least 75%) identity to SEQ ID NO: 31.

115. A nucleic acid comprising a nucleotide sequence encoding a chimeric C. acnes DsA1/DsA2/PITP polypeptide, wherein the chimeric C. acnes DsA1/DsA2/PITP polypeptide comprises an amino acid sequence comprising:
(i) a chimeric C. acnes DsA1/DsA2 polypeptide sequence which comprises the sequence of SEQ ID NO: 70, or a sequence having at least 90%, at least 91%, at least 92%, at least 93%, at least 94%, at least 95%, at least 96%, at least 97%, at least 98%, or at least 99% (e.g., at least 90%) identity to SEQ ID NO: 70; and
(ii) a C. acnes PITP polypeptide sequence;
and wherein (i) and (ii) are encoded on the same nucleic acid molecule.

116. The nucleic acid of embodiment 115, wherein the C. acnes PITP polypeptide sequence comprises an amino acid sequence corresponding to residues 1 to 133 of SEQ ID NO: 206 or a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g., at least 75%) identity thereto.

117. The nucleic acid of embodiment 116, wherein the C. acnes PITP polypeptide sequence consists of an amino acid sequence corresponding to residues 1 to 133 of SEQ ID NO: 206 or a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g., at least 75%) identity thereto.

118. The nucleic acid of embodiment 115 or 116, wherein the C. acnes PITP polypeptide sequence comprises an amino acid sequence corresponding to residues 1 to 146 of SEQ ID NO: 206 or a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g., at least 75%) identity thereto.

119. The nucleic acid of embodiment 115 or 116, wherein the C. acnes PITP polypeptide sequence consists of an amino acid sequence corresponding to residues 1 to 146 of SEQ ID NO: 206 or a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g., at least 75%) identity thereto.

120. The nucleic acid of any one of embodiments 115-119, wherein the nucleic acid comprises a sequence of any one of SEQ ID NO: 189 or 192 or a sequence having at least 50%, at least 75%, at least 80%, at least 85%, at least 90%, at least 91%, at least 92%, at least 93%, at least 94%, at least 95%, at least 96%, at least 97%, at least 98%, or at least 99% (e.g. at least 85%) identity to any one of SEQ ID NO: 189 or 192.

121 The nucleic acid of any one of embodiments 115-120, wherein the chimeric C. acnes DsA1/DsA2/PITP polypeptide further comprises a secretion signal peptide sequence (e.g., a viral a secretion signal peptide sequence).

122. The nucleic acid of any one of embodiments 115-121, wherein the chimeric C. acnes DsA1/DsA2/PITP polypeptide further comprises a heterologous transmembrane domain sequence.

123. A chimeric DsA1/DsA2/PITP polypeptide comprising an amino acid sequence comprising:
(i) a chimeric C. acnes DsA1/DsA2 polypeptide sequence comprising the sequence of SEQ ID NO: 70, or a sequence having at least 90%, at least 91%, at least 92%, at least 93%, at least 94%, at least 95%, at least 96%, at least 97%, at least 98%, or at least 99% (e.g., at least 90%) identity to SEQ ID NO: 70; and
(ii) a C. acnes PITP polypeptide sequence.

124. The chimeric DsA1/DsA2/PITP polypeptide of embodiment 123, wherein the C. acnes PITP polypeptide sequence comprises an amino acid sequence corresponding to residues 1 to 133 of SEQ ID NO: 206.

125. The chimeric DsA1/DsA2/PITP polypeptide of embodiment 123, wherein the C. acnes PITP polypeptide sequence consists of an amino acid sequence corresponding to residues 1 to 133 of SEQ ID NO: 206.

126. The chimeric DsA1/DsA2/PITP polypeptide of embodiment 123, wherein the C. acnes PITP polypeptide sequence comprises an amino acid sequence corresponding to residues 1 to 146 of SEQ ID NO: 206.

127. The chimeric DsA1/DsA2/PITP polypeptide of embodiment 123, wherein the C. acnes PITP polypeptide sequence consists of an amino acid sequence corresponding to residues 1 to 146 of SEQ ID NO: 206.

128. The chimeric DsA1/DsA2/PITP polypeptide of any one of embodiments 123-127, wherein the chimeric C. acnes DsA1/DsA2/PITP polypeptide comprises an amino acid sequence according to any one of SEQ ID NO: 80, 82 or 83 (e.g., SEQ ID NO: 80 or 83) or a sequence having at least 75%, at least 90%, at least 91%, at least 92%, at least 93%, at least 94%, at least 95%, at least 96%, at least 97%, at least 98%, or at least 99% (e.g., at least 95%) identity to any one of SEQ ID NO: 80, 82 or 83 (e.g., SEQ ID NO: 80 or 83).

129. The chimeric DsA1/DsA2/PITP polypeptide of any one of embodiments 123-128 wherein the chimeric C. acnes DsA1/DsA2/PITP polypeptide further comprises a heterologous transmembrane domain sequence.

130. The chimeric DsA1/DsA2/PITP polypeptide any one of embodiments 123-129, wherein (i) is a chimeric C. acnes DsA1/DsA2 polypeptide, optionally comprising the sequence of SEQ ID NO: 70, or a sequence having at least 90%, at least 91%, at least 92%, at least 93%, at least 94%, at least 95%, at least 96%, at least 97%, at least 98%, or at least 99% (e.g., at least 90%) identity to SEQ ID NO: 70.

131. The chimeric DsA1/DsA2/PITP polypeptide of any one of embodiments 123-130, wherein the polypeptide comprises a sequence of SEQ ID NO: 80, 82 or 83 (e.g., SEQ ID NO: 80 or 83) or a sequence having at least 50%, at least 90%, at least 91%, at least 92%, at least 93%, at least 94%, at least 95%, at least 96%, at least 97%, at least 98%, or at least 99% (e.g., at least 95%) identity thereto.

132. A composition comprising:
(i) a nucleic acid comprising a nucleotide sequence encoding a C. acnes DsA1 polypeptide;
(ii) a nucleic acid comprising a nucleotide sequence encoding a C. acnes DsA2 polypeptide; and
(iii) a nucleic acid comprising a nucleotide sequence encoding a C. acnes PITP polypeptide.

133. A composition comprising (i) a nucleic acid comprising a nucleotide sequence encoding a chimeric C. acnes DsA1/DsA2 polypeptide and (ii) a nucleic acid comprising a nucleotide sequence encoding a C. acnes PITP polypeptide, optionally wherein the nucleic acids are present in a weight ratio of 1:1.

134. A composition comprising a nucleic acid comprising a nucleotide sequence encoding a chimeric C. acnes DsA1/DsA2/PITP polypeptide.

135. The composition of any one of embodiments 133-134, wherein any two or more nucleic acids are located on the same nucleic acid molecule.

136. The composition of any one of embodiments 133-134, wherein any two or more nucleic acids are located on different nucleic acid molecules.

137. The composition of any one of embodiments 133-136, wherein the composition further comprises a lipid nanoparticle (LNP), optionally wherein any one or more nucleic acids are encapsulated in the LNP, e.g, wherein the LNP has an average diameter of 30 nm to 200 nm (e.g. an average diameter of 80 nm to 150 nm).

138. The composition of any one of embodiment 137, wherein the LNP comprises at least one cationic lipid, optionally wherein the cationic lipid
is biodegradable or is not biodegradable, and/or
the cationic lipid is cleavable or is not cleavable.

139. The composition of any one of embodiments 137-138, wherein the cationic lipid is selected from the group consisting of OF-02, cKK-E10, OF-Deg-Lin, GL-HEPES-E3-E10-DS-3-E18-1, GL-HEPES-E3-E12-DS-4-E10, GL-HEPES-E3-E12-DS-3-E14, SM-102, ALC-0315, IM-001 and IS-001.

140. The composition of any one of embodiments 137-139, wherein the cationic lipid is selected from the group consisting of OF-02, cKK-E10, GL-HEPES-E3-E12-DS-4-E10, IM-001 and IS-001 (e.g, wherein the cationic lipid is GL-HEPES-E3-E12-DS-4-E10).

141. The composition of any one of embodiments 137-140, wherein the LNP further comprises a polyethylene glycol (PEG) conjugated (PEGylated) lipid, a cholesterol-based lipid, and a helper lipid, optionally wherein the LNP comprises: a cationic lipid at a molar ratio of 35% to 55%; a polyethylene glycol (PEG) conjugated (PEGylated) lipid at a molar ratio of 0.25% to 2.75%; a cholesterol-based lipid at a molar ratio of 20% to 45%; and a helper lipid at a molar ratio of 5% to 35%, wherein all of the molar ratios are relative to the total lipid content of the LNP,
e.g, wherein the LNP comprises a cationic lipid at a molar ratio of 40%, a PEGylated lipid at a molar ratio of 1.5%, a cholesterol-based lipid at a molar ratio of 28.5%, and a helper lipid at a molar ratio of 30%.

142. The composition of embodiment 137-141, wherein any two or more nucleic acids are co-encapsulated in a single LNP.

143. The composition of any one of embodiments 137-142, wherein any two or more nucleic acids are encapsulated in separate LNPs.

144. The composition of any one of embodiments 133-143, wherein any one or more nucleic acids is a mRNA, optionally wherein each of the nucleic acids is a mRNA.

145. The composition of any one of embodiments 133-144, wherein the composition can be in a frozen liquid form or in a lyophilized form (e.g., in a lyophilized form).

146. A composition comprising:
(i) a C. acnes DsA1 polypeptide;
(ii) a C. acnes DsA2 polypeptide; and
(iii) a C. acnes PITP polypeptide.

147. A composition comprising a chimeric C. acnes DsA1/DsA2 polypeptide, and a C. acnes PITP polypeptide.

148. A composition comprising a chimeric C. acnes DsA1/DsA2/PITP polypeptide.

149. The composition of any one of embodiments 146-148, wherein the composition is an immunogenic composition.

150. The composition of any one of embodiments 146-149, wherein the composition further comprises an adjuvant.

151 The composition of embodiment 150, wherein the adjuvant is selected from the group consisting of: Aluminum based adjuvant (e.g. AlOOH), Squalene based oil in water emulsion adjuvants (e.g. AF03, AS03, MF59) and Liposome-based adjuvants comprising a saponin and a TLR4 agonist (e.g. SPA14, AS01, LEQ), e.g., wherein the adjuvant is selected from the group consisting of AlOOH, AF03 and SPA14.

152. The composition according to any one of embodiments 148-151, wherein the composition can be in a frozen liquid form or in a lyophilized form (e.g., in a lyophilized form).

153. The composition of any one of embodiments 133-152, wherein the C. acnes DsA1 polypeptide further comprises a secretion signal peptide sequence (e.g., a viral a secretion signal peptide sequence).

154. The composition of any one of embodiment 133-153, wherein the C. acnes DsA2 polypeptide further comprises a secretion signal peptide sequence (e.g., a viral a secretion signal peptide sequence).

155. The composition of any one of embodiment 133-154, wherein the C. acnes PITP polypeptide further comprises a secretion signal peptide sequence (e.g., a viral a secretion signal peptide sequence).

156 The composition any one of embodiments 133-155, wherein the C. acnes chimeric DsA1/DsA2 polypeptide further comprises a secretion signal peptide sequence (e.g., a viral a secretion signal peptide sequence).

157 The composition of any one of embodiments 133-153, wherein the C. acnes chimeric DsA1/DsA2/PITP polypeptide further comprises a secretion signal peptide sequence (e.g., a viral a secretion signal peptide sequence).

158. The composition of any one of embodiment 133-157, wherein the C. acnes DsA1 polypeptide further comprises a heterologous transmembrane domain sequence.

159. The composition of any one of embodiment 133-158, wherein the C. acnes DsA2 polypeptide further comprises a heterologous transmembrane domain sequence.

160 The composition of any one of embodiment 133-159, wherein the C. acnes PITP polypeptide further comprises a heterologous transmembrane domain sequence.

161. The composition of any one of embodiment 133-160, wherein the chimeric C. acnes DsA1/DsA2 polypeptide further comprises a heterologous transmembrane domain sequence.

162. The composition of any one of embodiment 133-161, wherein the chimeric C. acnes DsA1/DsA2/PITP polypeptide further comprises a heterologous transmembrane domain sequence.

163. A combination comprising:
(i) a nucleic acid comprising a nucleotide sequence encoding a C. acnes DsA1 polypeptide;
(ii) a nucleic acid comprising a nucleotide sequence encoding a C. acnes DsA2 polypeptide; and
(iii) a nucleic acid comprising a nucleotide sequence encoding a C. acnes PITP polypeptide.

164. A combination comprising (i) a nucleic acid comprising a nucleotide sequence encoding a chimeric C. acnes DsA1/DsA2 polypeptide and (ii) a nucleic acid comprising a nucleotide sequence encoding a C. acnes PITP polypeptide.

165. The combination of any one of embodiments 163 or 164, wherein any two or more nucleic acids are located on the same nucleic acid molecule.

166. The combination of any one of embodiments 163-164, wherein any two or more nucleic acids are located on different nucleic acid molecules.

167. The combination of any one of embodiments 163-166, wherein any one or more nucleic acids is a mRNA.

168. The combination of any one of embodiments 163-167, wherein each of the nucleic acids is a mRNA.

169. A combination comprising:
(i) a C. acnes DsA1 polypeptide;
(ii) a C. acnes DsA2 polypeptide; and
(iii) a C. acnes PITP polypeptide.

170. A combination comprising a chimeric C. acnes DsA1/DsA2 polypeptide, and a C. acnes PITP polypeptide.

171. The combination of embodiment 169 or 170 wherein the C. acnes DsA1 polypeptide further comprises a secretion signal peptide sequence (e.g., a viral a secretion signal peptide sequence).

172. The combination of any one of embodiments 169-171, wherein the C. acnes DsA2 polypeptide further comprises a secretion signal peptide sequence (e.g., a viral a secretion signal peptide sequence).

173. The combination of any one of embodiments 169-172, wherein the C. acnes PITP polypeptide further comprises a secretion signal peptide sequence (e.g., a viral a secretion signal peptide sequence).

174. The combination of any one of embodiments 169-173, wherein the C. acnes chimeric DsA1/DsA2 polypeptide further comprises a secretion signal peptide sequence (e.g., a viral a secretion signal peptide sequence).

175. The combination of any one of embodiments 169-174, wherein the C. acnes DsA1 polypeptide further comprises a heterologous transmembrane domain sequence.

176 The combination of any one of embodiments 169-175, wherein the C. acnes DsA2 polypeptide further comprises a heterologous transmembrane domain sequence.

177. The combination of embodiment any one of embodiments 169-176, wherein the C. acnes PITP polypeptide further comprises a heterologous transmembrane domain sequence.

178. The combination of any one of embodiments 169-177, wherein the chimeric C. acnes DsA1/DsA2 polypeptide further comprises a heterologous transmembrane domain sequence.

179. The combination of any one of embodiments 169-178, wherein the nucleic acids are present in the same composition.

180. The combination of any one of embodiments 169-178, wherein the nucleic acids are present in two or more separate compositions.

181. The combination of any one of embodiments 169-177, wherein the polypeptides are present in the same composition.

182. The combination of any one of embodiments 169-177, wherein the polypeptides are present in two or more separate compositions.

183. The nucleic acid of any one of the preceding embodiments, the composition of any one of the preceding embodiments or the combination of any one of the preceding embodiments, wherein the secretion signal peptide sequence comprises a sequence selected from the group consisting of: an influenza hemagglutinin (HA) secretion signal peptide sequence, a SARS CoV-2 spike secretion signal peptide sequence, a VZV gB secretion signal peptide sequence, a VZV gE secretion signal peptide sequence, a VZV gI secretion signal peptide sequence, a VZV gK secretion signal peptide sequence, a measles F-protein secretion signal peptide sequence, a rubella E1 protein secretion signal peptide sequence, a rubella E2 protein secretion signal peptide sequence, a mumps F-protein secretion signal peptide sequence, an Ebola GP protein secretion signal peptide sequence, a smallpox 6 kDa IC protein secretion signal peptide sequence and a rabies G protein secretion signal peptide sequence.

184. The nucleic acid of any one of the preceding embodiments, the composition of any one of the preceding embodiments or the combination of any one of the preceding embodiments wherein the nucleotide sequence encoding the secretion signal peptide sequence comprises a sequence according to any one of the sequences in Table 2 or Table 3.

185. The nucleic acid of any one of the preceding embodiments, the composition of any one of the preceding embodiments or the combination of any one of the preceding embodiments, wherein the secretion signal peptide sequence comprises an amino acid sequence according to any one of SEQ ID NO: 210-211.

186. The nucleic acid of any one of the preceding embodiments, the polypeptide of any one of the preceding embodiments, the composition of any one of the preceding embodiments or the combination of any one of the preceding embodiments, wherein the heterologous transmembrane domain comprises a viral transmembrane domain sequence, optionally wherein the viral transmembrane domain sequence is selected from the group consisting of: an influenza hemagglutinin (HA) transmembrane domain sequence, a SARS CoV-2 spike transmembrane domain sequence, a VZV gB transmembrane domain sequence, a VZV gE transmembrane domain sequence, a VZV gI transmembrane domain sequence, a VZV gK transmembrane domain sequence, a measles F-protein transmembrane domain sequence, a rubella E1 protein transmembrane domain sequence, a rubella E2 protein transmembrane domain sequence, a mumps F-protein transmembrane domain sequence, an Ebola GP protein transmembrane domain sequence and a rabies transmembrane domain sequence.

187. The nucleic acid of any one of the preceding embodiments, the polypeptide of any one of the preceding embodiments, the composition of any one of the preceding embodiments or the combination of any one of the preceding embodiments, wherein the transmembrane domain comprises an amino acid sequence according to any one of the sequences in Table 4.

188. The nucleic acid of any one of the preceding embodiments, the polypeptide of any one of the preceding embodiments, the composition of any one of the preceding embodiments or the combination of any one of the preceding embodiments, wherein the transmembrane domain sequence comprises an amino acid sequence according to any one of SEQ ID NO: 208-209.

189. The nucleic acid of any one of the preceding embodiments or the composition of any one of the preceding embodiments, wherein the mRNA comprises a 5′ cap, at least one 5′ untranslated region (5′ UTR), at least one 3′ untranslated region (3′ UTR), and/or at least one polyadenylation (poly(A)) sequence.

190. The nucleic acid of any one of the preceding embodiments or the composition of any one of the preceding embodiments, wherein the mRNA is unmodified.

191. The nucleic acid of any one of the preceding embodiments or the composition of any one of the preceding embodiments, wherein:
(i) the mRNA comprises at least one chemical modification, optionally wherein the chemical modification is selected from the group consisting of e.g., 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, phosphoramidates, phosphorothioates, peptide nucleotides, methylphosphonates, 7-deazaguanosine, 5-methylcytosine, and inosine (e.g., wherein the chemical modification comprises N1-methylpseudouridine), optionally wherein at least 20%, at least 30%, at least 40%, at least 50%, at least 60%, at least 70%, at least 80%, at least 85%, at least 90%, at least 95%, or 100% of the uridines in the mRNA are chemically modified); and/or
(ii) the mRNA is synthesized from modified nucleotide analogues or derivatives of purines and pyrimidines, optionally wherein the modified nucleotide analogues or derivatives of purines and pyrimidines are selected from the group consisting of: 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, phosphoramidates, phosphorothioates, peptide nucleotides, methylphosphonates, 7-deazaguanosine, 5-methylcytosine, and inosine.

192. The nucleic acid or the composition embodiment 191, wherein the chemical modification of the mRNA comprises N1-methylpseudouridine e.g. in place of every uridine.

193. The nucleic acid of any one of the preceding embodiments or the composition of any one of the preceding embodiments, wherein the mRNA is a self-replicating mRNA.

194. The nucleic acid of any one of the preceding embodiments or the composition of any one of the preceding embodiments, wherein the mRNA is a non-replicating mRNA.

195. The nucleic acid of any one of the preceding embodiments, the polypeptide of any one of the preceding embodiments, the composition of any one of the preceding embodiments, or the combination of any one of the preceding embodiments, wherein the C. acnes DsA1 polypeptide comprises a mutation at one or more (e.g., all) positions corresponding to an N-glycosylation site and/or a mutation at one or more (e.g., all) positions corresponding to an O-glycosylation site in the native C. acnes DsA1 polypeptide, optionally wherein the mutation is a single amino acid substitution.

196. The nucleic acid of any one of the preceding embodiments, the polypeptide of any one of the preceding embodiments, the composition of any one of the preceding embodiments, or the combination of any one of the preceding embodiments, wherein the C. acnes DsA1 polypeptide comprises a single amino acid substitution at one or more (e.g., all) positions corresponding to a cysteine residue in the native C. acnes DsA1 polypeptide, optionally wherein the single amino acid substitution is a substitution of cysteine with serine.

197. The nucleic acid of any one of the preceding embodiments, the polypeptide of any one of the preceding embodiments, the composition of any one of the preceding embodiments, or the combination of any one of the preceding embodiments, wherein the C. acnes DsA2 polypeptide comprises a mutation at one or more (e.g., all) positions corresponding to an N-glycosylation site and/or a mutation at one or more (e.g., all) positions corresponding to an O-glycosylation site in the native C. acnes DsA2 polypeptide, optionally wherein the mutation is a single amino acid substitution.

198. The nucleic acid of any one of the preceding embodiments, the polypeptide of any one of the preceding embodiments, the composition of any one of the preceding embodiments, or the combination of any one of the preceding embodiments, wherein the C. acnes DsA2 polypeptide comprises a single amino acid substitution at one or more (e.g., all) positions corresponding to a cysteine residue in the native C. acnes DsA2 polypeptide, optionally wherein the single amino acid substitution is a substitution of cysteine with serine.

199. The nucleic acid of any one of the preceding embodiments, the polypeptide of any one of the preceding embodiments, the composition of any one of the preceding embodiments, or the combination of any one of the preceding embodiments, wherein the C. acnes PITP polypeptide comprises a mutation at one or more (e.g., all) positions corresponding to an N-glycosylation site and/or a mutation at one or more (e.g., all) positions corresponding to an O-glycosylation site in the native C. acnes PITP polypeptide, optionally wherein the mutation is a single amino acid substitution.

200. The nucleic acid of any one of the preceding embodiments, the polypeptide of any one of the preceding embodiments, the composition of any one of the preceding embodiments, or the combination of any one of the preceding embodiments, wherein the C. acnes PITP polypeptide comprises a single amino acid substitution at one or more (e.g., all) positions corresponding to a cysteine residue in the native C. acnes PITP polypeptide, optionally wherein the single amino acid substitution is a substitution of cysteine with serine.

201. The nucleic acid of any one of the preceding embodiments, the polypeptide of any one of the preceding embodiments, the composition of any one of the preceding embodiments, or the combination of any one of the preceding embodiments, wherein the chimeric C. acnes DsA1/DsA2 polypeptide comprises a mutation at one or more (e.g., all) positions corresponding to an N-glycosylation site and/or a mutation at one or more (e.g., all) positions corresponding to an O-glycosylation site in the respective native C. acnes DsA1 polypeptide and/or in the respective native C. acnes DsA2 polypeptide, optionally wherein the mutation is a single amino acid substitution.

202. The nucleic acid of any one of the preceding embodiments, the polypeptide of any one of the preceding embodiments, the composition of any one of the preceding embodiments, or the combination of any one of the preceding embodiments, wherein the chimeric C. acnes DsA1/DsA2 polypeptide comprises a single amino acid substitution at one or more (e.g., all) positions corresponding to a cysteine residue in the respective native C. acnes DsA1 polypeptide and/or in in the respective native C. acnes DsA2 polypeptide, optionally wherein the single amino acid substitution is a substitution of cysteine with serine.

203. The nucleic acid of any one of the preceding embodiments, the polypeptide of any one of the preceding embodiments, the composition of any one of the preceding embodiments, or the combination of any one of the preceding embodiments, wherein the chimeric C. acnes DsA1/DsA2/PITP polypeptide comprises a mutation at one or more (e.g., all) positions corresponding to an N-glycosylation site and/or a mutation at one or more (e.g., all) positions corresponding to an O-glycosylation site in the respective native C. acnes DsA1 polypeptide, in the respective native C. acnes DsA2 polypeptide and/or in the respective native C. acnes PITP polypeptide, optionally wherein the mutation is a single amino acid substitution.

204. The nucleic acid of any one of the preceding embodiments, the polypeptide of any one of the preceding embodiments, the composition of any one of the preceding embodiments, or the combination of any one of the preceding embodiments, wherein the chimeric C. acnes DsA1/DsA2/PITP polypeptide comprises a single amino acid substitution at one or more (e.g., all) positions corresponding to a cysteine residue in the respective native C. acnes DsA1 polypeptide, in the respective native C. acnes DsA2 polypeptide and/or in the respective native C. acnes PITP polypeptide, optionally wherein the single amino acid substitution is a substitution of cysteine with serine.

205. The nucleic acid of any one of the preceding embodiments, the polypeptide of any one of the preceding embodiments, the composition of any one of the preceding embodiments, or the combination of any one of the preceding embodiments, wherein the C. acnes CAMP2 polypeptide comprises a mutation at one or more (e.g., all) positions corresponding to an N-glycosylation site and/or a mutation at one or more (e.g., all) positions corresponding to an O-glycosylation site in the respective native C. acnes CAMP2 polypeptide, optionally wherein the mutation is a single amino acid substitution.

206. The nucleic acid of any one of the preceding embodiments, the polypeptide of any one of the preceding embodiments, the composition of any one of the preceding embodiments, or the combination of any one of the preceding embodiments, wherein the C. acnes CAMP2 polypeptide comprises a single amino acid substitution at one or more (e.g., all) positions corresponding to a cysteine residue in the respective native C. acnes CAMP2 polypeptide, optionally wherein the single amino acid substitution is a substitution of cysteine with serine.

207. The composition of any one of the preceding embodiments or the combination of any one of the preceding embodiments, wherein the C. acnes DsA1 polypeptide comprises the sequence of any one of SEQ ID NO: 204, SEQ ID NO: 17-19 or SEQ ID NO: 59-61, or a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g., at least 75%) identity to any one of SEQ ID NO: 204, SEQ ID NO: 17-19 or SEQ ID NO: 59-61.

208. The composition of any one of the preceding embodiments or the combination of any one of the preceding embodiments, wherein the C. acnes DsA2 polypeptide comprises the sequence of any one of SEQ ID NO: 205, SEQ ID NO: 20-27 or SEQ ID NO: 62-69, or a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g., at least 75%) identity to any one of SEQ ID NO: 205, SEQ ID NO: 20-27 or SEQ ID NO: 62-69.

209. The composition of any one of the preceding embodiments or the combination of any one of the preceding embodiments, wherein the C. acnes PITP polypeptide comprises the sequence according to any one of SEQ ID NO: 206, SEQ ID NO: 31-37 or SEQ ID NO: 73-79, or a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g., at least 75%) identity to any one of SEQ ID NO: 206, SEQ ID NO: 31-37 or SEQ ID NO: 73-79.

210. The composition of any one of the preceding embodiments or the combination of any one of the preceding embodiments, wherein the chimeric C. acnes DsA1/DsA2 polypeptide comprises the sequence of SEQ ID NO: 28-30, SEQ ID NO: 39 or SEQ ID NO: 70-72 or SEQ ID NO: 81 (e.g., SEQ ID NO: 70), or a sequence having at least 90%, at least 91%, at least 92%, at least 93%, at least 94%, at least 95%, at least 96%, at least 97%, at least 98%, or at least 99% (e.g., at least 90%) identity to any one of SEQ ID NO: 28-30, SEQ ID NO: 39 or SEQ ID NO: 70-72 or SEQ ID NO: 81.

211. The composition of any one of the preceding embodiments or the combination of any one of the preceding embodiments, wherein the chimeric C. acnes DsA1/DsA2/PITP polypeptide comprises the sequence of SEQ ID NO: 80, 82 or 83 (e.g., SEQ ID NO: 80 or 83), or a sequence having at least 90%, at least 91%, at least 92%, at least 93%, at least 94%, at least 95%, at least 96%, at least 97%, at least 98%, or at least 99% (e.g., at least 75%) identity to SEQ ID NO: 80, 82 or 83 (e.g., SEQ ID NO: 80 or 83).

212. The composition of any one of the preceding embodiments or the combination of any one of the preceding embodiments, wherein the C. acnes CAMP2 polypeptide comprises the sequence of SEQ ID NO: 203, or a sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% (e.g., at least 60%, such as at least 85%) identity to SEQ ID NO: 203.

213. An mRNA comprising or consisting of (e.g. consisting of) the following structural elements:
(i) a 5′ cap with the following structure:

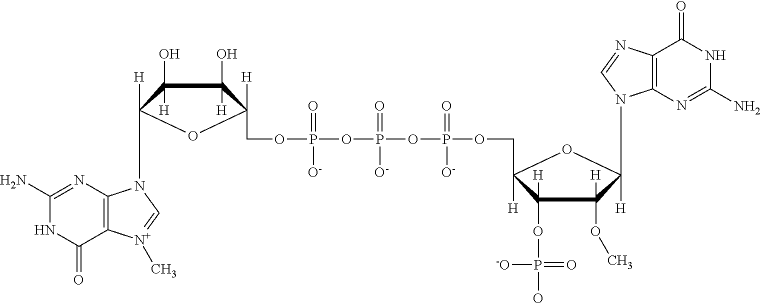

(ii) a 5′ untranslated region (5′ UTR) having the nucleic acid sequence according to SEQ ID NO: 265;
(iii) a protein coding region having the nucleic acid sequence according to any one of SEQ ID NO: 90-91, or SEQ ID NO: 391-392 (e.g., SEQ ID NO: 91);
(iv) a 3′ untranslated region (3′ UTR) having the nucleic acid sequence according to SEQ ID NO: 266; and
(v) a polyA tail, optionally wherein the polyA tail comprises at least 75 adenosine nucleotides (such as about 80 adenosine nucleotides) or at least 100 adenosine nucleotides (such as about 115 adenosine nucleotides), e.g., wherein the polyA tail comprises at least 100 adenosine nucleotides.

214. The mRNA of embodiment 213, wherein the mRNA is chemically modified and wherein the chemical modification comprises or consist of (e.g. consists of) N1-methylpseudouridine in place of every uridine.

215. The mRNA of embodiments 213 or 214, wherein the 3′ end of (i) bonds directly to the 5′ end of (ii) via a 3′ to 5′ phosphodiester linkage; the 3′ end of (ii) bonds directly to the 5′ end of (iii) via a 3′ to 5′ phosphodiester linkage; the 3′ end of (iii) bonds directly to the 5′ end of (iv) via a 3′ to 5′ phosphodiester linkage; and the 3′ end of (iv) bonds directly to the 5′ end of (v) via a 3′ to 5′ phosphodiester linkage.

216. An mRNA comprising or consisting of (e.g. consisting of) the following structural elements:
(i) a 5′ cap with the following structure:

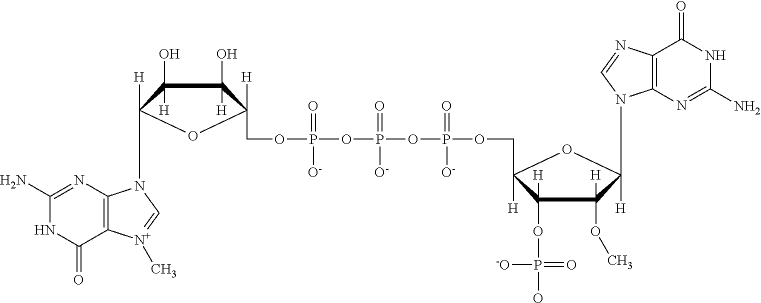

(ii) a 5′ untranslated region (5′ UTR) having the nucleic acid sequence according to SEQ ID NO: 265;
(iii) a protein coding region having the nucleic acid sequence according to SEQ ID NO: 113;
(iv) a 3′ untranslated region (3′ UTR) having the nucleic acid sequence according to SEQ ID NO: 266; and
(v) a polyA tail, optionally wherein the polyA tail comprises at least 75 adenosine nucleotides (such as about 80 adenosine nucleotides) or at least 100 adenosine nucleotides (such as about 115 adenosine nucleotides), e.g., wherein the polyA tail comprises at least 100 adenosine nucleotides.

217. The mRNA of embodiment 216, wherein the mRNA is chemically modified and wherein the chemical modification comprises or consist of (e.g. consists of) N1-methylpseudouridine in place of every uridine.

218. The mRNA of embodiment 216 or 217, wherein the 3′ end of (i) bonds directly to the 5′ end of (ii) via a 3′ to 5′ phosphodiester linkage; the 3′ end of (ii) bonds directly to the 5′ end of (iii) via a 3′ to 5′ phosphodiester linkage; the 3′ end of (iii) bonds directly to the 5′ end of (iv) via a 3′ to 5′ phosphodiester linkage; and the 3′ end of (iv) bonds directly to the 5′ end of (v) via a 3′ to 5′ phosphodiester linkage.

219 An mRNA comprising or consisting of (e.g. consisting of) the following structural elements:
(i) a 5′ cap with the following structure:

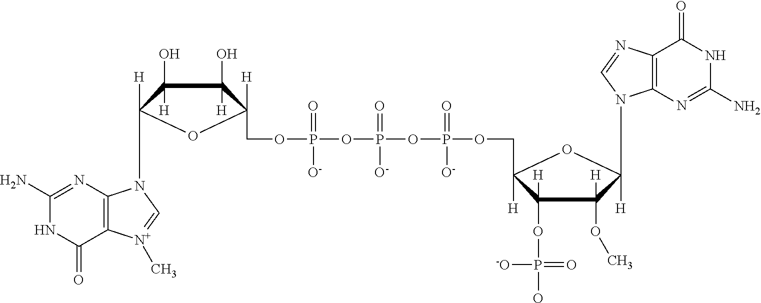

(ii) a 5′ untranslated region (5′ UTR) having the nucleic acid sequence according to SEQ ID NO: 265;
(iii) a protein coding region having the nucleic acid sequence according to SEQ ID NO: 115;
(iv) a 3′ untranslated region (3′ UTR) having the nucleic acid sequence according to SEQ ID NO: 266; and
(v) a polyA tail, optionally wherein the polyA tail comprises at least 75 adenosine nucleotides (such as about 80 adenosine nucleotides) or at least 100 adenosine nucleotides (such as about 115 adenosine nucleotides), e.g., wherein the polyA tail comprises at least 100 adenosine nucleotides.

220. The mRNA of embodiment 219, wherein the mRNA is chemically modified and wherein the chemical modification comprises or consist of (e.g. consists of) N1-methylpseudouridine in place of every uridine.

221. The mRNA of embodiment 219 or 220, wherein the 3′ end of (i) bonds directly to the 5′ end of (ii) via a 3′ to 5′ phosphodiester linkage; the 3′ end of (ii) bonds directly to the 5′ end of (iii) via a 3′ to 5′ phosphodiester linkage; the 3′ end of (iii) bonds directly to the 5′ end of (iv) via a 3′ to 5′ phosphodiester linkage; and the 3′ end of (iv) bonds directly to the 5′ end of (v) via a 3′ to 5′ phosphodiester linkage.

222. A LNP encapsulating the mRNA of any one of embodiments 213-215.

223. A LNP encapsulating the mRNA of any one of embodiments 216-218.

224. A LNP encapsulating the mRNA of any one of embodiment 219-221.

225. A LNP encapsulating the mRNA of any one of embodiments 213-215 and the mRNA of any one of embodiments 216-218, optionally wherein the mRNAs are present in a weight ratio of 1:1.

226. A LNP encapsulating the mRNA of any one of embodiments 213-215 and the mRNA of any one of embodiments 219-221, optionally wherein the mRNAs are present in a weight ratio of 1:1.

227. A LNP encapsulating the mRNA of any one of embodiments 216-218 and the mRNA of any one of embodiments 219-221, optionally wherein the mRNAs are present in a weight ratio of 1:1.

228. A LNP encapsulating the mRNA of any one of embodiments 213-215, the mRNA of any one of embodiments 216-218 and the mRNA of any one of embodiments 219-221, optionally wherein the mRNAs are present in a weight ratio of 1:1:1.

229. A composition (e.g. an immunogenic composition) comprising the mRNA according to any one of embodiments 213-221.

230. The composition of embodiment 229, wherein the composition comprises:
(i) the mRNA of any one of embodiments 213-215 and the mRNA of any one of embodiments 216-218, optionally wherein the mRNAs are present in a weight ratio of 1:1, further optionally wherein the mRNAs are co-encapsulated in the same LNP;
(ii) the mRNA of any one of embodiments 216-218 and the mRNA of any one of embodiments 219-221, optionally wherein the mRNAs are present in a weight ratio of 1:1, further optionally wherein the mRNAs are co-encapsulated in the same LNP;
(iii) the mRNA of any one of embodiments 213-215 and the mRNA of any one of embodiments 219-221, optionally wherein the mRNAs are present in a weight ratio of 1:1, further optionally wherein the mRNAs are co-encapsulated in the same LNP; or
(iv) the mRNA of any one of embodiments 213-215, the mRNA of any one of embodiments 216-218 and the mRNA of any one of embodiments 219-221, optionally wherein the mRNAs are present in a weight ratio of 1:1:1, further optionally wherein the mRNAs are co-encapsulated in the same LNP.

231. The composition of embodiment 229 or 230, wherein the composition comprises the mRNA of embodiment 213 and the mRNA of embodiment 216, optionally wherein each mRNA comprises at least one chemical modification (e.g., wherein at least 20%, at least 30%, at least 40%, at least 50%, at least 60%, at least 70%, at least 80%, at least 85%, at least 90%, at least 95%, or 100% of the uridines in the mRNA are chemically modified), e.g, wherein the chemical modification comprises N1-methylpseudouridine, preferably wherein all uridines are substituted with N1-methylpseudouridine.

232. The composition of embodiment 229 or 230, wherein the composition comprises the mRNA of embodiment 213 and the mRNA of embodiment 219, optionally wherein each mRNA comprises at least one chemical modification (e.g., wherein at least 20%, at least 30%, at least 40%, at least 50%, at least 60%, at least 70%, at least 80%, at least 85%, at least 90%, at least 95%, or 100% of the uridines in the mRNA are chemically modified), e.g, wherein the chemical modification comprises N1-methylpseudouridine, preferably wherein all uridines are substituted with N1-methylpseudouridine.

233. The composition of embodiment 229 or 230, wherein the composition comprises the mRNA of embodiment 216 and the mRNA of embodiment 219, optionally wherein each mRNA comprises at least one chemical modification (e.g., wherein at least 20%, at least 30%, at least 40%, at least 50%, at least 60%, at least 70%, at least 80%, at least 85%, at least 90%, at least 95%, or 100% of the uridines in the mRNA are chemically modified), e.g, wherein the chemical modification comprises N1-methylpseudouridine, preferably wherein all uridines are substituted with N1-methylpseudouridine.

234. A composition (e.g. an immunogenic composition) comprising the LNPs according to any one of embodiments 222-228.

235. The composition of embodiment 229 or 230 or the LNP of any one of embodiments 222-228, wherein:
(i) the LNP comprises at least one cationic lipid, optionally wherein the cationic lipid
   is biodegradable or is not biodegradable, and/or
   the cationic lipid is cleavable or is not cleavable; and
(ii) the cationic lipid is selected from the group consisting of OF-02, cKK-E10, GL-HEPES-E3-E12-DS-4-E10, IM-001 and IS-001, optionally wherein the cationic lipid is GL-HEPES-E3-E12-DS-4-E10.

236. The composition or the LNP of embodiment 235, wherein the LNP comprises GL-HEPES-E3-E12-DS-4-E10 at a molar ratio of 40%, DMG-PEG2000 at a molar ratio of 1.5%, cholesterol at a molar ratio of 28.5% and DOPE at a molar ratio of 30%.

237. The composition according to any one of embodiments 29-60, 132-145, 183-212 or 229-236, wherein the composition comprises 1-300 μg of the one or more nucleic acid(s) (e.g. mRNA(s)), such as about 2.5 μg, about 5 μg, about 7.5 μg, about 10 μg, about 12.5 μg, about 15 μg, about 30 μg, about 45 μg, about 60 μg, about 75 μg, about 90 μg, about 105 μg, about 120 μg, about 135 μg, about 150 μg, about 165 μg, about 180 μg, about 195 μg, about 210 μg, or about 225 μg of the one or more nucleic acid(s) (e.g., mRNA(s)).

238. The composition according to embodiment 237, wherein the composition comprises 40-50 μg (e.g., about 45 μg) of the one or more nucleic acid(s) (e.g. mRNA(s)).

239. The composition according to embodiment 237, wherein the composition comprises 110-140 μg (e.g., about 120 μg) of the one or more nucleic acid(s) (e.g. mRNA(s)).

240. The composition according to embodiment 237, wherein the composition comprises 200-250 μg (e.g., about 225 μg) of the one or more nucleic acids of mRNA(s)).

241. A vaccine composition comprising the nucleic acid (e.g. mRNA) of any one of the preceding embodiments, the polypeptide of any one of the preceding embodiments or the combination of any one of the preceding embodiments.

242. The nucleic acid (e.g. mRNA) of any one of the preceding embodiments, the polypeptide of any one of the preceding embodiments, the composition of any one of the preceding embodiments, or the combination of any one of the preceding embodiments for use as a medicament.

243. The use of the nucleic acid (e.g. mRNA) of any one of the preceding embodiments, the polypeptide of any one of the preceding embodiments, the composition of any one of the preceding embodiments, or the combination of any one of the preceding embodiments for the manufacture of a medicament.

244. A method of treating or preventing a disease comprising administering the nucleic acid (e.g. mRNA) of any one of the preceding embodiments, the polypeptide of any one of the preceding embodiments, the composition of any one of the preceding embodiments, or the combination of any one of the preceding embodiments to a subject in need thereof.

245. The nucleic acid (e.g. mRNA) of any one of the preceding embodiments, the polypeptide of any one of the preceding embodiments, the composition of any one of the preceding embodiments, or the combination of any one of the preceding embodiments for use in a method of treating or preventing C. acnes infection (e.g. a moderate or severe C. acnes infection) in a subject (e.g. human).

246. The use of the nucleic acid (e.g. mRNA) of any one of the preceding embodiments, the polypeptide of any one of the preceding embodiments, the composition of any one of the preceding embodiments, or the combination of any one of the preceding embodiments for the manufacture of a medicament for treating or preventing C. acnes infection (e.g. a moderate or severe C. acnes infection) in a subject (e.g. human).

247. A method of treating or preventing a C. acnes infection (e.g. a moderate or severe C. acnes infection) comprising administering the nucleic acid (e.g. mRNA) of any one of the preceding embodiments, the polypeptide of any one of the preceding embodiments, the composition of any one of the preceding embodiments, or the combination of any one of the preceding embodiments to a subject (e.g. human) in need thereof.

248. The nucleic acid (e.g. mRNA) of any one of the preceding embodiments, the polypeptide of any one of the preceding embodiments, the composition of any one of the preceding embodiments, or the combination of any one of the preceding embodiments for use in a method of treating or preventing acne (e.g. mild, moderate or severe acne) in a subject (e.g. human).

249. The use of the nucleic acid (e.g. mRNA) of any one of the preceding embodiments, the polypeptide of any one of the preceding embodiments, the composition of any one of the preceding embodiments, or the combination of any one of the preceding embodiments for the manufacture of a medicament for treating or preventing acne (e.g. mild, moderate or severe acne) in a subject (e.g. human).

250. A method of treating or preventing acne (e.g. mild, moderate or severe acne) comprising administering nucleic acid (e.g. mRNA) of any one of the preceding embodiments, the polypeptide of any one of the preceding embodiments, the composition of any one of the preceding embodiments, or the combination of any one of the preceding embodiments to a subject (e.g. human) in need thereof.

251. The nucleic acid (e.g. mRNA) of any one of the preceding embodiments, the polypeptide of any one of the preceding embodiments, the composition of any one of the preceding embodiments, or the combination of any one of the preceding embodiments for use in a method of eliciting an immune response in a subject, optionally wherein the subject is a human subject.

252. The use of nucleic acid (e.g. mRNA) of any one of the preceding embodiments, the polypeptide of any one of the preceding embodiments, the composition of any one of the preceding embodiments, or the combination of any one of the preceding embodiments for the manufacture of a medicament for eliciting an immune response in a subject, optionally wherein the subject is a human subject.

253. A method of eliciting an immune response in a subject comprising administering nucleic acid (e.g. mRNA) of any one of the preceding embodiments, the polypeptide of any one of the preceding embodiments, the composition of any one of the preceding embodiments, or the combination of any one of the preceding embodiments to the subject.

254. The method of any one of embodiments 244, 247, 250 or 253, wherein the method comprises:
(i) administering one dose of the nucleic acid, the polypeptide, the composition, or the combination; or
(ii) administering two doses of the nucleic acid, the polypeptide, the composition, or the combination, optionally wherein the two doses are administered two months apart
e.g, wherein the administration is according to (ii).

255. The nucleic acid, the polypeptide, the composition, or the combination for use according to any one of embodiments 242, 245, 248 or 251, wherein the method comprises:
(i) administering one dose of the nucleic acid, the polypeptide, the composition, or the combination; or
(ii) administering two doses of the nucleic acid, the polypeptide, the composition, or the combination, optionally wherein the two doses are administered two months apart.

256. The use of the nucleic acid, the polypeptide, the composition, or the combination according to any one of embodiments 243, 246, 249, or 252, wherein the treating or preventing comprises:
(i) administering one dose of the nucleic acid, the polypeptide, the composition, or the combination; or
(ii) administering two doses of the nucleic acid, the polypeptide, the composition, or the combination, optionally wherein the two doses are administered two months apart.

257. The method of any one of embodiments 244, 247, 250, 253 or 254, the nucleic acid, the polypeptide, the composition, or the combination for use according to any one of embodiments 242, 245, 248, 251 or 255, or the use of the nucleic acid, the polypeptide, the composition, or the combination of any one of embodiments 243, 246, 249, 252 or 256, wherein the method comprises administering the polypeptide, nucleic acid, composition or combination
(i) intramuscularly; or
(ii) through skin injection (e.g. in the epidermis, the dermis or the hypodermis of the skin), such as intradermally, optionally with a device suitable for skin injection, such as a needle (e.g. an epidermic, dermic or hypodermic needle), a needle free device, a microneedle device or a microprojection array device.

258. The method of any one of embodiments 244, 247, 250, 253, 254 or 257, the nucleic acid, the polypeptide, the composition, or the combination for use according to any one of embodiments 242, 245, 248, 251, 255 or 257, or the use of the nucleic acid, the polypeptide, the composition, or the combination of any one of embodiments 243, 246, 249, 252, 256 or 257, wherein the subject is a human subject and wherein the subject is between 9 and 45 years old or between 12 and 45 years old (e.g. between 18 and 45 years old).

259. An mRNA comprising or consisting of (e.g. consisting of) the following structural elements:
(i) a 5′ cap with the following structure:

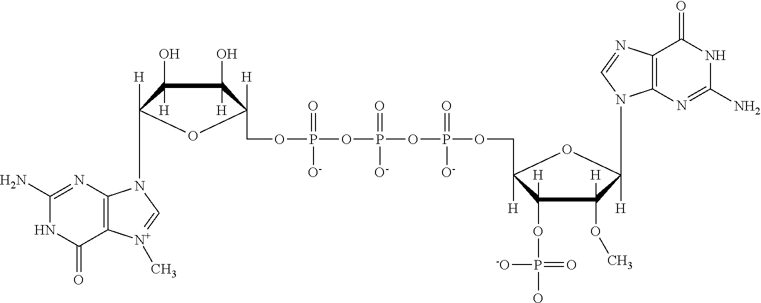

(ii) a 5′ untranslated region (5′ UTR) having the nucleic acid sequence according to SEQ ID NO: 265;
(iii) a protein coding region having the nucleic acid sequence according to any one of SEQ ID NO: 87-89;
(iv) a 3′ untranslated region (3′ UTR) having the nucleic acid sequence according to SEQ ID NO: 266; and
(v) a polyA tail.

260. The mRNA of embodiment 259, wherein the mRNA is chemically modified and wherein the chemical modification comprises or consist of (e.g. consists of) N1-methylpseudouridine in place of every uridine.

261. The mRNA of embodiment 259 or 260, wherein the 3′ end of (i) bonds directly to the 5′ end of (ii) via a 3′ to 5′ phosphodiester linkage; the 3′ end of (ii) bonds directly to the 5′ end of (iii) via a 3′ to 5′ phosphodiester linkage; the 3′ end of (iii) bonds directly to the 5′ end of (iv) via a 3′ to 5′ phosphodiester linkage; and the 3′ end of (iv) bonds directly to the 5′ end of (v) via a 3′ to 5′ phosphodiester linkage.

262. A LNP encapsulating the mRNA of embodiments 259-261.

263. A nucleic acid comprising a nucleotide sequence encoding a chimeric C. acnes DsA1/DsA2/PITP/CAMP2 polypeptide, wherein the chimeric C. acnes DsA1/DsA2/PITP/CAMP2 polypeptide comprises:
(a) a chimeric C. acnes DsA1/DsA2 polypeptide, e.g., as described in any one of embodiments 97-105;
(b) an immunogenic fragment of a C. acnes PITP polypeptide, optionally wherein the immunogenic fragment comprises a ENFD of a C. acnes PITP polypeptide; and
(c) a C. acnes CAMP2 polypeptide or an immunogenic fragment thereof.

264. The nucleic acid of embodiment 263, wherein the chimeric C. acnes DsA1/DsA2 polypeptide comprises:
(A) (1) a CSD2 of a C. acnes DsA1 polypeptide and (2) a CSD1 and/or a CSD3 of a C. acnes DsA2 polypeptide; or
(B) (1) a CSD2 of a C. acnes DsA2 polypeptide and (2) a CSD1 and/or a CSD3 of a C. acnes DsA1 polypeptide

265. The nucleic acid of embodiment 264, wherein the chimeric C. acnes DsA1/DsA2/PITP/CAMP2 polypeptide is as defined in (B).

266. The nucleic acid of any one of embodiments 263-265, wherein the chimeric C. acnes DsA1/DsA2/PITP/CAMP2 polypeptide comprises a CSD1 of a C. acnes DsA1 polypeptide, a CSD2 of a C. acnes DsA2 polypeptide and a CSD3 of a C. acnes DsA1 polypeptide.

267. The nucleic acid of any one of embodiments 263-266, wherein the chimeric C. acnes DsA1/DsA2 polypeptide of (a) comprises a sequence according to SEQ ID NO: 70, or a sequence having at least 90%, at least 91%, at least 92%, at least 93%, at least 94%, at least 95%, at least 96%, at least 97%, at least 98%, or at least 99% (e.g., at least 90%) identity thereto.

268. The nucleic acid of any one of embodiments 263-267, wherein the immunogenic fragment of a C. acnes PITP polypeptide comprising a ENFD of a C. acnes PITP polypeptide in (b) comprises the sequence corresponding to amino acid residues 1-133 of SEQ ID NO: 73 or a sequence having at least 90%, at least 91%, at least 92%, at least 93%, at least 94%, at least 95%, at least 96%, at least 97%, at least 98%, or at least 99% (e.g., at least 90%) identity thereto.

269. The nucleic acid of embodiment 268, wherein the immunogenic fragment of a C. acnes PITP polypeptide comprising a ENFD of a C. acnes PITP polypeptide in (b) comprises the sequence corresponding to amino acid residues 1-146 of SEQ ID NO: 73 or a sequence having at least 90%, at least 91%, at least 92%, at least 93%, at least 94%, at least 95%, at least 96%, at least 97%, at least 98%, or at least 99% (e.g., at least 90%) identity thereto.

270. The nucleic acid of any one of embodiments 263-269, wherein (a) and (b) comprise SEQ ID NO: 80, 82 or 83 or a sequence having at least 90%, at least 91%, at least 92%, at least 93%, at least 94%, at least 95%, at least 96%, at least 97%, at least 98%, or at least 99% (e.g., at least 90%) identity thereto.

271. The nucleic acid of embodiment 270, wherein (a) and (b) comprise SEQ ID NO: 80 or a sequence having at least 90%, at least 91%, at least 92%, at least 93%, at least 94%, at least 95%, at least 96%, at least 97%, at least 98%, or at least 99% (e.g., at least 90%) identity thereto.

272. The nucleic acid of any one of embodiments 263-269, wherein (c) is a C. acnes CAMP2 polypeptide, optionally wherein (c) comprises SEQ ID NO: 203 or a sequence having at least 90%, at least 91%, at least 92%, at least 93%, at least 94%, at least 95%, at least 96%, at least 97%, at least 98%, or at least 99% (e.g., at least 90%) identity thereto.

273. The nucleic acid of embodiment 272, wherein:
(i) the chimeric C. acnes DsA1/DsA2/PITP/CAMP2 polypeptide comprises a sequence according to SEQ ID NO: 373 or 374 or a sequence having at least 90%, at least 91%, at least 92%, at least 93%, at least 94%, at least 95%, at least 96%, at least 97%, at least 98%, or at least 99% (e.g., at least 90%) identity thereto; and/or
(ii) the nucleotide sequence encoding the chimeric C. acnes DsA1/DsA2/PITP/CAMP2 polypeptide comprises a sequence according to any one of SEQ ID NO: 384-387 (e.g., SEQ ID NO: 385 or 387) or a sequence having at least 75%, at least 80%, at least 85%, at least 90%, at least 91%, at least 92%, at least 93%, at least 94%, at least 95%, at least 96%, at least 97%, at least 98%, or at least 99% (e.g., at least 75%) identity thereto; and/or
(iii) the nucleotide sequence encoding the chimeric C. acnes DsA1/DsA2/PITP/CAMP2 polypeptide comprises a sequence according to any one of SEQ ID NO: 377-380 (e.g., SEQ ID NO: 378 or 380) or a sequence having at least 75%, at least 80%, at least 85%, at least 90%, at least 91%, at least 92%, at least 93%, at least 94%, at least 95%, at least 96%, at least 97%, at least 98%, or at least 99% (e.g., at least 75%) identity thereto.

274. The nucleic acid of any one of embodiments 263-269, wherein the immunogenic fragment of a C. acnes CAMP2 polypeptide in (c) comprises a N-terminal domain of a C. acnes CAMP2 polypeptide, e.g, wherein the immunogenic fragment of a C. acnes CAMP2 polypeptide in (c) comprises amino acid residues 29-176 of SEQ ID NO: 202 or a sequence having at least 90%, at least 91%, at least 92%, at least 93%, at least 94%, at least 95%, at least 96%, at least 97%, at least 98%, or at least 99% (e.g., at least 90%) identity to the sequence of amino acid residues 29-176 of SEQ ID NO: 202.

275. The nucleic acid of any one of embodiments 263-269, wherein the immunogenic fragment of a C. acnes CAMP2 polypeptide in (c) comprises a N-terminal domain of a C. acnes CAMP2 polypeptide and a linker domain of a C. acnes CAMP2 polypeptide.

276. The nucleic acid of embodiment 275, wherein the immunogenic fragment of a C. acnes CAMP2 polypeptide in (c) comprises amino acid residues 29-188 of SEQ ID NO: 202 or a sequence having at least 90%, at least 91%, at least 92%, at least 93%, at least 94%, at least 95%, at least 96%, at least 97%, at least 98%, or at least 99% (e.g., at least 90%) identity thereto.

277. The nucleic acid of embodiment 276, wherein:
(i) the chimeric C. acnes DsA1/DsA2/PITP/CAMP2 polypeptide comprises a sequence according to SEQ ID NO: 375 or a sequence having at least 90%, at least 91%, at least 92%, at least 93%, at least 94%, at least 95%, at least 96%, at least 97%, at least 98%, or at least 99% (e.g., at least 90%) identity thereto; and/or
(ii) the nucleotide sequence encoding the chimeric C. acnes DsA1/DsA2/PITP/CAMP2 polypeptide comprises a sequence according to any one of SEQ ID NO: 388-389 (e.g., SEQ ID NO: 389) or a sequence having at least 75%, at least 80%, at least 85%, at least 90%, at least 91%, at least 92%, at least 93%, at least 94%, at least 95%, at least 96%, at least 97%, at least 98%, or at least 99% (e.g., at least 75%) identity thereto; and/or
(iii) the nucleotide sequence encoding the chimeric C. acnes DsA1/DsA2/PITP/CAMP2 polypeptide comprises a sequence according to any one of SEQ ID NO: 381-382 (e.g., SEQ ID NO: 382) or a sequence having at least 75%, at least 80%, at least 85%, at least 90%, at least 91%, at least 92%, at least 93%, at least 94%, at least 95%, at least 96%, at least 97%, at least 98%, or at least 99% (e.g., at least 75%) identity thereto.

278. The nucleic acid of any one of embodiments 263-269, wherein the immunogenic fragment of a C. acnes CAMP2 polypeptide in (c) comprises a C-terminal domain of a C. acnes CAMP2 polypeptide.

279. The nucleic acid of embodiment 278, wherein the immunogenic fragment of a C. acnes CAMP2 polypeptide in (c) comprises amino acid residues 189-267 of SEQ ID NO: 202 or a sequence having at least 90%, at least 91%, at least 92%, at least 93%, at least 94%, at least 95%, at least 96%, at least 97%, at least 98%, or at least 99% (e.g., at least 90%) identity thereto.

280. The nucleic acid of embodiment 279, wherein:
(i) the chimeric C. acnes DsA1/DsA2/PITP/CAMP2 polypeptide comprises a sequence according to SEQ ID NO: 376 or a sequence having at least 90%, at least 91%, at least 92%, at least 93%, at least 94%, at least 95%, at least 96%, at least 97%, at least 98%, or at least 99% (e.g., at least 90%) identity thereto; and/or
(ii) the nucleotide sequence encoding the chimeric C. acnes DsA1/DsA2/PITP/CAMP2 polypeptide comprises a sequence according to any one of SEQ ID NO: 390 or 393 (e.g., SEQ ID NO: 390) or a sequence having at least 75%, at least 80%, at least 85%, at least 90%, at least 91%, at least 92%, at least 93%, at least 94%, at least 95%, at least 96%, at least 97%, at least 98%, or at least 99% (e.g., at least 75%) identity thereto; and/or
(iii) the nucleotide sequence encoding the chimeric C. acnes DsA1/DsA2/PITP/CAMP2 polypeptide comprises a sequence according to any one of SEQ ID NO: 383 or 394 (e.g., SEQ ID NO: 383) or a sequence having at least 75%, at least 80%, at least 85%, at least 90%, at least 91%, at least 92%, at least 93%, at least 94%, at least 95%, at least 96%, at least 97%, at least 98%, or at least 99% (e.g., at least 75%) identity thereto.

281. The nucleic acid of any one of embodiments 263-269, wherein the immunogenic fragment of a C. acnes CAMP2 polypeptide in (c) comprises a linker domain of a C. acnes CAMP2 polypeptide and a C-terminal domain of a C. acnes CAMP2 polypeptide, e.g, wherein the immunogenic fragment of a C. acnes CAMP2 polypeptide in (c) comprises amino acid residues 177-267 of SEQ ID NO: 202 or a sequence having at least 90%, at least 91%, at least 92%, at least 93%, at least 94%, at least 95%, at least 96%, at least 97%, at least 98%, or at least 99% (e.g., at least 90%) identity to the sequence of amino acid residues 177-267 of SEQ ID NO: 202.

282. The nucleic acid of any one of embodiments 263-281, wherein any two or more of (a), (b) and (c) are attached via linkers or are directly fused to each other (e.g., wherein any two or more of (a), (b) and (c) are directly fused to each other).

283. The nucleic acid of any one of embodiments 263-282, wherein the chimeric C. acnes DsA1/DsA2/PITP/CAMP2 polypeptide comprises, from N-terminus to C-terminus:
(a), (b) and (c); or
(c), (a) and (b),
e.g., wherein wherein the chimeric C. acnes DsA1/DsA2/PITP/CAMP2 polypeptide comprises, from N-terminus to C-terminus: (c), (a) and (b).

284. The nucleic acid of any one of embodiments 263-283, wherein the chimeric C. acnes DsA1/DsA2/PITP/CAMP2 polypeptide comprises a transmembrane domain sequence.

285. The nucleic acid of embodiment 284, wherein the transmembrane sequence is positioned at the N terminus or the C terminus (e.g. the C terminus) of the chimeric C. acnes DsA1/DsA2/PITP/CAMP2 polypeptide.

286. The nucleic acid of embodiment 285, wherein the transmembrane domain sequence comprises an amino acid sequence according to any one of the sequences in Table 4 or SEQ ID NO: 84 (e.g., SEQ ID NO: 208 or 84).

287. The nucleic acid of embodiment 286, wherein:
(i) the chimeric C. acnes DsA1/DsA2/PITP/CAMP2 polypeptide comprises a sequence according to SEQ ID NO: 373 or a sequence having at least 90%, at least 91%, at least 92%, at least 93%, at least 94%, at least 95%, at least 96%, at least 97%, at least 98%, or at least 99% (e.g., at least 90%) identity thereto and a transmembrane domain sequence (e.g., SEQ ID NO: 84); and/or
(ii) the nucleotide sequence encoding the chimeric C. acnes DsA1/DsA2/PITP/CAMP2 polypeptide comprises a sequence according to SEQ ID NO: 384, or a sequence having at least 75%, at least 80%, at least 85%, at least 90%, at least 91%, at least 92%, at least 93%, at least 94%, at least 95%, at least 96%, at least 97%, at least 98%, or at least 99% (e.g., at least 75%) identity thereto, and a nucleotide sequence encoding a transmembrane domain sequence (e.g., a sequence according to SEQ ID NO: 395), or
   the nucleotide sequence encoding the chimeric C. acnes DsA1/DsA2/PITP/CAMP2 polypeptide comprises a sequence according to SEQ ID NO: 385, or a sequence having at least 75%, at least 80%, at least 85%, at least 90%, at least 91%, at least 92%, at least 93%, at least 94%, at least 95%, at least 96%, at least 97%, at least 98%, or at least 99% (e.g., at least 75%) identity thereto, and a nucleotide sequence encoding a transmembrane domain sequence (e.g., a sequence according to SEQ ID NO: 396),
   e.g., wherein the nucleotide sequence encoding the chimeric C. acnes DsA1/DsA2/PITP/CAMP2 polypeptide comprises a sequence according to SEQ ID NO: 385 and a sequence according to SEQ ID NO: 396; and/or
(iii) the nucleotide sequence encoding the chimeric C. acnes DsA1/DsA2/PITP/CAMP2 polypeptide comprises a sequence according to SEQ ID NO: 377 or 378 or a sequence having at least 75%, at least 80%, at least 85%, at least 90%, at least 91%, at least 92%, at least 93%, at least 94%, at least 95%, at least 96%, at least 97%, at least 98%, or at least 99% (e.g., at least 75%) identity thereto (e.g., the chimeric C. acnes DsA1/DsA2/PITP/CAMP2 polypeptide comprises a sequence according to SEQ ID NO: 378).

288. The nucleic acid of any one of embodiments 263-287, wherein the chimeric C. acnes DsA1/DsA2/PITP/CAMP2 polypeptide comprises a non-native (e.g. viral) secretion signal peptide sequence.

289. The nucleic acid of embodiment 288, wherein the secretion signal peptide sequence is positioned at the N-terminus or the C-terminus (e.g. the N-terminus) of the chimeric C. acnes DsA1/DsA2/PITP/CAMP2 polypeptide.

290. The nucleic acid of embodiment 288 or 289, wherein the secretion signal peptide sequence is a viral secretion signal peptide sequence, which optionally comprises a sequence selected from the group consisting of: an influenza hemagglutinin (HA) secretion signal peptide sequence, a SARS CoV-2 spike secretion signal peptide sequence, a VZV gB secretion signal peptide sequence, a VZV gE secretion signal peptide sequence, a VZV gI secretion signal peptide sequence, a VZV gK secretion signal peptide sequence, a measles F-protein secretion signal peptide sequence, a rubella E1 protein secretion signal peptide sequence, a rubella E2 protein secretion signal peptide sequence, a mumps F-protein secretion signal peptide sequence, an Ebola GP protein secretion signal peptide sequence, and a smallpox 6 kDa IC protein secretion signal peptide sequence.

291. The nucleic acid of any one of embodiments 288-290, wherein the secretion signal peptide sequence comprises a sequence according to any one of the sequences in Table 2 or 3.

292. The nucleic acid of any one of embodiments 288-291, wherein the secretion signal peptide sequence comprises a sequence according to any one of SEQ ID NO: 210-211 (e.g., SEQ ID NO: 210).

293. The nucleic acid of any one of embodiments 263-287, wherein the nucleic acid is mRNA.

294. A chimeric C. acnes DsA1/DsA2/PITP/CAMP2 polypeptide comprising:
(a) a chimeric C. acnes DsA1/DsA2 polypeptide, e.g., as described in any one of embodiments 97-105;
(b) an immunogenic fragment of a C. acnes PITP polypeptide, optionally wherein the immunogenic fragment comprises a ENFD of a C. acnes PITP polypeptide; and
(c) a C. acnes CAMP2 polypeptide or an immunogenic fragment thereof.

295. The chimeric C. acnes DsA1/DsA2/PITP/CAMP2 polypeptide of embodiment 294, wherein the chimeric C. acnes DsA1/DsA2 polypeptide comprises:
(A) (1) a CSD2 of a C. acnes DsA1 polypeptide and (2) a CSD1 and/or a CSD3 of a C. acnes DsA2 polypeptide; or
(B) (1) a CSD2 of a C. acnes DsA2 polypeptide and (2) a CSD1 and/or a CSD3 of a C. acnes DsA1 polypeptide

296. The chimeric C. acnes DsA1/DsA2/PITP/CAMP2 polypeptide of embodiment 295, wherein the chimeric C. acnes DsA1/DsA2/PITP/CAMP2 polypeptide is as defined in (B).

297. The chimeric C. acnes DsA1/DsA2/PITP/CAMP2 polypeptide of any one of embodiments 294-296, wherein the chimeric C. acnes DsA1/DsA2/PITP/CAMP2 polypeptide comprises a CSD1 of a C. acnes DsA1 polypeptide, a CSD2 of a C. acnes DsA2 polypeptide and a CSD3 of a C. acnes DsA1 polypeptide.

298. The chimeric C. acnes DsA1/DsA2/PITP/CAMP2 polypeptide of any one of embodiments 294-297, wherein the chimeric C. acnes DsA1/DsA2 polypeptide of (a) comprises a sequence according to SEQ ID NO: 70, or a sequence having at least 90%, at least 91%, at least 92%, at least 93%, at least 94%, at least 95%, at least 96%, at least 97%, at least 98%, or at least 99% (e.g., at least 90%) identity thereto.

299. The chimeric C. acnes DsA1/DsA2/PITP/CAMP2 polypeptide of any one of embodiments 294-298, wherein the immunogenic fragment of a C. acnes PITP polypeptide comprising a ENFD of a C. acnes PITP polypeptide in (b) comprises the sequence corresponding to amino acid residues 1-133 of SEQ ID NO: 73 or a sequence having at least 90%, at least 91%, at least 92%, at least 93%, at least 94%, at least 95%, at least 96%, at least 97%, at least 98%, or at least 99% (e.g., at least 90%) identity thereto.

300. The chimeric C. acnes DsA1/DsA2/PITP/CAMP2 polypeptide of embodiment 299, wherein the immunogenic fragment of a C. acnes PITP polypeptide comprising a ENFD of a C. acnes PITP polypeptide in (b) comprises the sequence corresponding to amino acid residues 1-146 of SEQ ID NO: 73 or a sequence having at least 90%, at least 91%, at least 92%, at least 93%, at least 94%, at least 95%, at least 96%, at least 97%, at least 98%, or at least 99% (e.g., at least 90%) identity thereto.

301. The chimeric C. acnes DsA1/DsA2/PITP/CAMP2 polypeptide of any one of embodiments 294-300, wherein (a) and (b) comprise SEQ ID NO: 80, 82 or 83 or a sequence having at least 90%, at least 91%, at least 92%, at least 93%, at least 94%, at least 95%, at least 96%, at least 97%, at least 98%, or at least 99% (e.g., at least 90%) identity thereto.

302. The chimeric C. acnes DsA1/DsA2/PITP/CAMP2 polypeptide of embodiment 301, wherein (a) and (b) comprise SEQ ID NO: 80 or a sequence having at least 90%, at least 91%, at least 92%, at least 93%, at least 94%, at least 95%, at least 96%, at least 97%, at least 98%, or at least 99% (e.g., at least 90%) identity thereto.

303. The chimeric C. acnes DsA1/DsA2/PITP/CAMP2 polypeptide of any one of embodiments 294-302, wherein (c) is a C. acnes CAMP2 polypeptide, optionally wherein (c) comprises SEQ ID NO: 203 or a sequence having at least 90%, at least 91%, at least 92%, at least 93%, at least 94%, at least 95%, at least 96%, at least 97%, at least 98%, or at least 99% (e.g., at least 90%) identity thereto.

304. The chimeric C. acnes DsA1/DsA2/PITP/CAMP2 polypeptide of embodiment 303, wherein the chimeric C. acnes DsA1/DsA2/PITP/CAMP2 polypeptide comprises a sequence according to SEQ ID NO: 373 or 374 or a sequence having at least 90%, at least 91%, at least 92%, at least 93%, at least 94%, at least 95%, at least 96%, at least 97%, at least 98%, or at least 99% (e.g., at least 90%) identity thereto.

305. The chimeric C. acnes DsA1/DsA2/PITP/CAMP2 polypeptide of any one of embodiments 294-302, wherein the immunogenic fragment of a C. acnes CAMP2 polypeptide in (c) comprises a N-terminal domain of a C. acnes CAMP2 polypeptide, e.g, wherein the immunogenic fragment of a C. acnes CAMP2 polypeptide in (c) comprises amino acid residues 29-176 of SEQ ID NO: 202 or a sequence having at least 90%, at least 91%, at least 92%, at least 93%, at least 94%, at least 95%, at least 96%, at least 97%, at least 98%, or at least 99% (e.g., at least 90%) identity to the sequence of amino acid residues 29-176 of SEQ ID NO: 202.

306. The chimeric C. acnes DsA1/DsA2/PITP/CAMP2 polypeptide of any one of embodiments 294-302, wherein the immunogenic fragment of a C. acnes CAMP2 polypeptide in (c) comprises a N-terminal domain of a C. acnes CAMP2 polypeptide and a linker domain of a C. acnes CAMP2 polypeptide.

307. The chimeric C. acnes DsA1/DsA2/PITP/CAMP2 polypeptide of embodiment 306, wherein the immunogenic fragment of a C. acnes CAMP2 polypeptide in (c) comprises amino acid residues 29-188 of SEQ ID NO: 202 or a sequence having at least 90%, at least 91%, at least 92%, at least 93%, at least 94%, at least 95%, at least 96%, at least 97%, at least 98%, or at least 99% (e.g., at least 90%) identity thereto.

308. The chimeric C. acnes DsA1/DsA2/PITP/CAMP2 polypeptide of embodiment 307, wherein the chimeric C. acnes DsA1/DsA2/PITP/CAMP2 polypeptide comprises a sequence according to SEQ ID NO: 375 or a sequence having at least 90%, at least 91%, at least 92%, at least 93%, at least 94%, at least 95%, at least 96%, at least 97%, at least 98%, or at least 99% (e.g., at least 90%) identity thereto.

309 The chimeric C. acnes DsA1/DsA2/PITP/CAMP2 polypeptide of any one of embodiments 294-302, wherein the immunogenic fragment of a C. acnes CAMP2 polypeptide in (c) comprises a C-terminal domain of a C. acnes CAMP2 polypeptide.

310. The chimeric C. acnes DsA1/DsA2/PITP/CAMP2 polypeptide of embodiment 309, wherein the immunogenic fragment of a C. acnes CAMP2 polypeptide in (c) comprises amino acid residues 189-267 of SEQ ID NO: 202 or a sequence having at least 90%, at least 91%, at least 92%, at least 93%, at least 94%, at least 95%, at least 96%, at least 97%, at least 98%, or at least 99% (e.g., at least 90%) identity thereto.

311. The chimeric C. acnes DsA1/DsA2/PITP/CAMP2 polypeptide of embodiment 310, wherein the chimeric C. acnes DsA1/DsA2/PITP/CAMP2 polypeptide comprises a sequence according to SEQ ID NO: 376 or a sequence having at least 90%, at least 91%, at least 92%, at least 93%, at least 94%, at least 95%, at least 96%, at least 97%, at least 98%, or at least 99% (e.g., at least 90%) identity thereto.

312. The chimeric C. acnes DsA1/DsA2/PITP/CAMP2 polypeptide of any one of embodiments 294-302, wherein the immunogenic fragment of a C. acnes CAMP2 polypeptide in (c) comprises a linker domain of a C. acnes CAMP2 polypeptide and a C-terminal domain of a C. acnes CAMP2 polypeptide, e.g, wherein the immunogenic fragment of a C. acnes CAMP2 polypeptide in (c) comprises amino acid residues 177-267 of SEQ ID NO: 202 or a sequence having at least 90%, at least 91%, at least 92%, at least 93%, at least 94%, at least 95%, at least 96%, at least 97%, at least 98%, or at least 99% (e.g., at least 90%) identity to the sequence of amino acid residues 177-267 of SEQ ID NO: 202.

313. The chimeric C. acnes DsA1/DsA2/PITP/CAMP2 polypeptide of any one of embodiments 294-312, wherein any two or more of (a), (b) and (c) are attached via linkers or are directly fused to each other (e.g., wherein any two or more of (a), (b) and (c) are directly fused to each other).

314. The chimeric C. acnes DsA1/DsA2/PITP/CAMP2 polypeptide of any one of embodiments 294-313, wherein the chimeric C. acnes DsA1/DsA2/PITP/CAMP2 polypeptide comprises, from N-terminus to C-terminus:
(a), (b) and (c); or
(c), (a) and (b),
e.g., wherein wherein the chimeric C. acnes DsA1/DsA2/PITP/CAMP2 polypeptide comprises, from N-terminus to C-terminus: (c), (a) and (b).

315. The chimeric C. acnes DsA1/DsA2/PITP/CAMP2 polypeptide of any one of embodiments 294-314, wherein the chimeric C. acnes DsA1/DsA2/PITP/CAMP2 polypeptide comprises a transmembrane domain sequence.

316. The chimeric C. acnes DsA1/DsA2/PITP/CAMP2 polypeptide of embodiment 315, wherein the transmembrane sequence is positioned at the N terminus or the C terminus (e.g. the C terminus) of the chimeric C. acnes DsA1/DsA2/PITP/CAMP2 polypeptide.

317. The chimeric C. acnes DsA1/DsA2/PITP/CAMP2 polypeptide of embodiment 316, wherein the transmembrane domain sequence comprises an amino acid sequence according to any one of the sequences in Table 4 or SEQ ID NO: 84 (e.g., SEQ ID NO: 84).

318. The chimeric C. acnes DsA1/DsA2/PITP/CAMP2 polypeptide of embodiment 317, wherein the chimeric C. acnes DsA1/DsA2/PITP/CAMP2 polypeptide comprises a sequence according to SEQ ID NO: 373 or a sequence having at least 90%, at least 91%, at least 92%, at least 93%, at least 94%, at least 95%, at least 96%, at least 97%, at least 98%, or at least 99% (e.g., at least 90%) identity thereto and a transmembrane domain sequence (e.g., SEQ ID NO: 84).

319. A LNP encapsulating the mRNA of embodiment 293, optionally wherein the LNP has an average diameter of 30 nm to 200 nm (e.g. an average diameter of 80 nm to 150 nm).

320. A composition, optionally an immunogenic composition, comprising the nucleic acid (e.g., a mRNA) according to any one of embodiments 263-293.

321. The composition of embodiments 320, wherein the composition further comprises a lipid nanoparticle (LNP), optionally wherein the nucleic acid is encapsulated in the LNP, optionally wherein the LNP has an average diameter of 30 nm to 200 nm (e.g. an average diameter of 80 nm to 150 nm).

322. The composition of any one of embodiment 319 or 321, wherein the LNP comprises at least one cationic lipid, optionally wherein the cationic lipid
is biodegradable or is not biodegradable, and/or
the cationic lipid is cleavable or is not cleavable.

323. The composition of embodiment 322, wherein the cationic lipid is selected from the group consisting of OF-02, cKK-E10, OF-Deg-Lin, GL-HEPES-E3-E10-DS-3-E18-1, GL-HEPES-E3-E12-DS-4-E10, GL-HEPES-E3-E12-DS-3-E14, SM-102, ALC-0315, IS-001 and IM-001.

324. The composition of embodiment 323, wherein the cationic lipid is selected from the group consisting of OF-02, cKK-E10, GL-HEPES-E3-E12-DS-4-E10, IS-001 and IM-001 (e.g, wherein the cationic lipid is GL-HEPES-E3-E12-DS-4-E10).

325. The composition of any one of embodiments 319 or 321-324, wherein the LNP further comprises a polyethylene glycol (PEG) conjugated (PEGylated) lipid, a cholesterol-based lipid, and a helper lipid, optionally wherein the LNP comprises: a cationic lipid at a molar ratio of 35% to 55%; a polyethylene glycol (PEG) conjugated (PEGylated) lipid at a molar ratio of 0.25% to 2.75%; a cholesterol-based lipid at a molar ratio of 20% to 45%; and a helper lipid at a molar ratio of 5% to 35%, wherein all of the molar ratios are relative to the total lipid content of the LNP, e.g, wherein the LNP comprises a cationic lipid at a molar ratio of 40%, a PEGylated lipid at a molar ratio of 1.5%, a cholesterol-based lipid at a molar ratio of 28.5%, and a helper lipid at a molar ratio of 30%.

326. The composition of any one of embodiments 320-325, wherein the nucleic acid is mRNA.

327. The composition of any one of embodiments 326, wherein the mRNA comprises a 5′ cap, at least one 5′ untranslated region (5′ UTR), at least one 3′ untranslated region (3′ UTR), and/or at least one polyadenylation (poly(A)) sequence.

328. The composition of any one of embodiments 326-327, wherein the mRNA is unmodified or:
(i) the mRNA comprises at least one chemical modification, optionally wherein the chemical modification is selected from the group consisting of e.g., 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, phosphoramidates, phosphorothioates, peptide nucleotides, methylphosphonates, 7-deazaguanosine, 5-methylcytosine, and inosine (e.g., wherein the chemical modification comprises N1-methylpseudouridine); and/or
(ii) the mRNA is synthesized from modified nucleotide analogues or derivatives of purines and pyrimidines, optionally wherein the modified nucleotide analogues or derivatives of purines and pyrimidines are selected from the group consisting of: 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, phosphoramidates, phosphorothioates, peptide nucleotides, methylphosphonates, 7-deazaguanosine, 5-methylcytosine, and inosine.

329. The composition of any one of embodiments 326-328, wherein the mRNA comprises at least one chemical modification (e.g., wherein at least 20%, at least 30%, at least 40%, at least 50%, at least 60%, at least 70%, at least 80%, at least 85%, at least 90%, at least 95%, or 100% of the uridines in the mRNA are chemically modified), optionally wherein the chemical modification comprises N1-methylpseudouridine e.g. in place of every uridine.

330. The composition of any one of embodiments 326-329, wherein the mRNA is a self-replicating mRNA or a non-replicating mRNA, e.g, wherein the mRNA is a non-replicating mRNA.

331. The composition of any one of embodiments 326-330, wherein the composition can be in a frozen liquid form or in a lyophilized form (e.g., in a lyophilized form).

332. A composition, optionally an immunogenic composition, comprising the chimeric C. acnes DsA1/DsA2/PITP/CAMP2 polypeptide as defined in any one of embodiments 294-318, optionally wherein the composition can be in a frozen liquid form or in a lyophilized form (e.g., in a lyophilized form).

333. The composition of embodiment 332, wherein the composition further comprises an adjuvant, optionally wherein the adjuvant is selected from the group consisting of: Aluminum based adjuvant (e.g. AlOOH), Squalene based oil in water emulsion adjuvants (e.g. AF03, AS03, MF59) and Liposome-based adjuvants comprising a saponin and a TLR4 agonist (e.g. SPA14, AS01, LEQ).

334. A vaccine composition comprising the nucleic acid (e.g. mRNA) of any one of embodiments 263-293 or the polypeptide of any one of embodiments 294-318.

335. The nucleic acid (e.g. mRNA) of any one of embodiments 263-293, the polypeptide of any one of embodiments 294-318, the LNP of embodiment 319 or the composition of any one of embodiments 320-333 for use as a medicament.

336. The use of the nucleic acid (e.g. mRNA) of any one of embodiments 263-293, the polypeptide of any one of embodiments 294-318, the LNP of embodiment 319 or the composition of any one of embodiments 320-333 for the manufacture of a medicament.

337. A method of treating or preventing a disease comprising administering the nucleic acid (e.g. mRNA) of any one of embodiments 263-293, the polypeptide of any one of embodiments 294-318, the LNP of embodiment 319 or the composition of any one of embodiments 320-333 to a subject in need thereof.

338. The nucleic acid (e.g. mRNA) of any one of embodiments 263-293, the polypeptide of any one of embodiments 294-318, the LNP of embodiment 319 or the composition of any one of embodiments 320-333 for use in a method of treating or preventing C. acnes infection (e.g. a moderate or severe C. acnes infection) in a subject (e.g. human).

339. The use of the nucleic acid (e.g. mRNA) of any one of embodiments 263-293, the polypeptide of any one of embodiments 294-318, the LNP of embodiment 319 or the composition of any one of embodiments 320-333 for the manufacture of a medicament for treating or preventing C. acnes infection (e.g. a moderate or severe C. acnes infection) in a subject (e.g. human).

340. A method of treating or preventing a C. acnes infection (e.g. a moderate or severe C. acnes infection) comprising administering the nucleic acid (e.g. mRNA) of any one of embodiments 263-293, the polypeptide of any one of embodiments 294-318, the LNP of embodiment 319 or the composition of any one of embodiments 320-333 to a subject (e.g. human) in need thereof.

341. The nucleic acid (e.g. mRNA) of any one of embodiments 263-293, the polypeptide of any one of embodiments 294-318, the LNP of embodiment 319 or the composition of any one of embodiments 320-333 for use in a method of treating or preventing acne (e.g. mild, moderate or severe acne) in a subject (e.g. human).

342. The use of the nucleic acid (e.g. mRNA) of any one of embodiments 263-293, the polypeptide of any one of embodiments 294-318, the LNP of embodiment 319 or the composition of any one of embodiments 320-333 for the manufacture of a medicament for treating or preventing acne (e.g. mild, moderate or severe acne) in a subject (e.g. human).

343. A method of treating or preventing acne (e.g. mild, moderate or severe acne) comprising administering nucleic acid (e.g. mRNA) of any one of embodiments 263-293, the polypeptide of any one of embodiments 294-318, the LNP of embodiment 319 or the composition of any one of embodiments 320-333 to a subject (e.g. human) in need thereof.

344. The nucleic acid (e.g. mRNA) of any one of embodiments 263-293, the polypeptide of any one of embodiments 294-318, the LNP of embodiment 319 or the composition of any one of embodiments 320-333 for use in a method of eliciting an immune response in a subject, optionally wherein the subject is a human subject.

345. The use of nucleic acid (e.g. mRNA) of any one of embodiments 263-293, the polypeptide of any one of embodiments 294-318, the LNP of embodiment 319 or the composition of any one of embodiments 320-333 for the manufacture of a medicament for eliciting an immune response in a subject, optionally wherein the subject is a human subject.

346. A method of eliciting an immune response in a subject comprising administering nucleic acid (e.g. mRNA) of any one of embodiments 263-293, the polypeptide of any one of embodiments 294-318, the LNP of embodiment 319 or the composition of any one of embodiments 320-333 to the subject.

347. The method of any one of embodiments 337, 340, 343 or 346, wherein the method comprises:
(i) administering one dose of the nucleic acid, the polypeptide or the composition; or
(ii) administering two doses of the nucleic acid, the polypeptide or the composition, optionally wherein the two doses are administered two months apart e.g, wherein the administration is according to (ii).

348. The nucleic acid, the polypeptide or the composition for use according to any one of embodiments 335, 338, 341 or 344, wherein the method comprises:
(i) administering one dose of the nucleic acid, the polypeptide or the composition; or
(ii) administering two doses of the nucleic acid, the polypeptide or the composition, optionally wherein the two doses are administered two months apart.

349. The use of the nucleic acid, the polypeptide or the composition according to any one of embodiments 336, 339, 342 or 345, wherein the treating or preventing comprises:
(i) administering one dose of the nucleic acid, the polypeptide or the composition; or
(ii) administering two doses of the nucleic acid, the polypeptide or the composition, optionally wherein the two doses are administered two months apart.

350. The method of any one of embodiments 337, 340, 343, 346 or 347, the nucleic acid, the polypeptide or the composition for use according to any one of embodiments 335, 338, 341, 344 or 348, or the use of the nucleic acid, the polypeptide or the composition of any one of embodiments 336, 339, 342, 345 or 349, wherein the method comprises administering the polypeptide, nucleic acid or the composition intramuscularly.

351. The method of any one of embodiments 337, 340, 343, 346 or 347, the nucleic acid, the polypeptide or the composition for use according to any one of embodiments 335, 338, 341, 344 or 348, or the use of the nucleic acid, the polypeptide or the composition of any one of embodiments 336, 339, 342, 345 or 349, wherein the method comprises administering the polypeptide, nucleic acid or the composition through skin injection (e.g. in the epidermis, the dermis or the hypodermis of the skin), such as intradermally, optionally with a device suitable for skin injection, such as a needle (e.g. an epidermic, dermic or hypodermic needle), a needle free device, a microneedle device or a microprojection array device.

352. The method of any one of embodiments 337, 340, 343, 346, 347, 350 or 351, the nucleic acid, the polypeptide, the composition, or the combination for use according to any one of embodiments 335, 338, 341, 344, 348, 350 or 351, or the use of the nucleic acid, the polypeptide, the composition, or the combination of any one of embodiments 336, 339, 342, 345 or 349-351, wherein the subject is a human subject and wherein the subject is between 9 and 45 years old or between 12 and 45 years old (e.g. between 18 and 45 years old).

### BRIEF DESCRIPTION OF THE DRAWINGS

FIG. 1 shows co-hemolytic neutralizing titers of IgG purified from sera of mice immunized twice at 3 weeks interval with different mRNA constructs encoding C. acnes CAMP2 polypeptides (encapsulated in a LNP comprising ckk-E10 as cationic lipid) or a recombinant C. acnes CAMP2 polypeptides (formulated in either AlOOH or SPA14 adjuvants). LNP alone, and adjuvants alone (AlOOH and SPA14) served as negative controls.

FIG. 2 shows Surface Binding (SB) and Opsonophagocytic Killing (OPK) titers from either individual sera (SB) or pool of sera (OPK), performed on NCTC737 C. acnes strain, of mice immunized twice at 4 weeks interval. Experimental formulations contained either H4-V3 mRNA antigen construct (encapsulated in an LNP comprising GL-HEPES-E3-E12-DS-4-E10 as cationic lipid) or H4-V3 recombinant protein (formulated in AlOOH). LNP alone served as a negative control. LLOQ=lowest limit of quantification.

FIGS. 3A-3B show Surface Binding (SB) and Opsonophagocytic Killing (OPK) titers from either individual sera (SB) or pool of sera (OPK), performed on NCTC737 (FIG. 3a) and KPA171202 (FIG. 3b) C. acnes strains, of mice immunized twice at 4 weeks interval. Experimental formulations contained either P028-V7 mRNA antigen construct (encapsulated in an LNP comprising GL-HEPES-E3-E12-DS-4-E10 as cationic lipid) or P028-V7 recombinant protein (formulated in AlOOH). LNP alone served as a negative control. LLOQ=lowest limit of quantification.

FIG. 4 shows a schematic visualization of CAMP2 mRNA constructs that have been designed and synthesized.

FIG. 5 shows characterization data of proteins expressed in HEK cells after in vitro cell transfection with mRNA constructs encoding C. acnes CAMP2 polypeptides (construct design shown in FIG. 4).

FIG. 6 shows co-hemolytic neutralizing titers of IgG purified from sera of mice immunized twice at 4 weeks interval with mRNA constructs encoding C. acnes CAMP2 polypeptides (construct design shown in FIG. 4). LNP alone served as a negative control. LLOQ=lowest limit of quantification.

FIG. 7 shows ELISA anti-CAMP2 IgG titers of sera of mice immunized twice at 4 weeks interval with mRNA constructs encoding C. acnes CAMP2 polypeptides (construct design shown in FIG. 4). LNP alone served as a negative control. LLOQ=lowest limit of quantification.

FIGS. 8A-8B show ELISA anti-DSA1 IgG titers from individual sera of mice immunized twice at 4 weeks interval with a range of mRNA constructs encoding C. acnes DsA1 polypeptides, C. acnes DsA2 polypeptides and chimeric C. acnes DsA1/DsA2 polypeptides, and containing either a secretion signal (FIG. 8a) or both a secretion signal and a transmembrane domain (FIG. 8b), and encapsulated in an LNP comprising GL-HEPES-E3-E12-DS-4-E10 as cationic lipid. LNP alone served as a negative control. A formulation containing 10 μg of H4-V3 recombinant protein formulated in AlOOH was used as a benchmark.

FIGS. 9A-9B show ELISA anti-DSA2 IgG titers from individual sera of mice immunized twice at 4 weeks interval with the same mRNA antigens as described in FIGS. 8A-8B, containing either a secretion signal (FIG. 9a) or both a secretion signal and a transmembrane domain (FIG. 9b), and encapsulated in the same LNP. LNP alone served as a negative control. A formulation containing 10 μg of H4-V3 recombinant protein formulated in AlOOH was used as a benchmark.

FIGS. 10A-10B show Surface Binding (SB) titers from pools of sera of mice immunized twice at 4 weeks interval with the same mRNA antigens as described in FIG. 8A-8B, containing either a secretion signal (FIG. 10a) or both a secretion signal and a transmembrane domain, except for the H4-V3 construct (FIG. 10b), and encapsulated in the same LNP. LNP alone served as a negative control. A formulation containing 10 μg of H4-V3 recombinant protein formulated in AlOOH was used as a benchmark. The SB assay was performed on NCTC737 C. acnes strain. LLOQ=lowest limit of quantification.

FIGS. 11A-11B show Opsonophagocytic Killing (OPK) titers from pools of sera of mice immunized twice at 4 weeks interval with the same mRNA antigens as described in FIGS. 8A-8B, containing either a secretion signal (FIG. 11a) or both a secretion signal and a transmembrane domain, except for the H4-V3 construct (FIG. 11b), and encapsulated in the same LNP. LNP alone served as a negative control. A formulation containing 10 μg of H4-V3 recombinant protein formulated in AlOOH was used as a benchmark. The OPK assay was performed on NCTC737 C. acnes strains. LLOQ=lowest limit of quantification.

FIGS. 12A-12B show ELISA anti-PITP IgG titers from individual sera of mice immunized twice at 4 weeks interval, with a range of mRNA constructs encoding C. acnes PITP antigens, and containing either a secretion signal (FIG. 12a) or both a secretion signal and a transmembrane domain (FIG. 12b), encapsulated in an LNP comprising GL-HEPES-E3-E12-DS-4-E10 as cationic lipid. LNP alone was used as negative control. A formulation containing 10 μg of P028-V7 recombinant protein formulated in AlOOH was used as a benchmark.

FIGS. 13A-13B show Surface Binding (SB) titers from pools of sera of mice immunized twice at 4 weeks interval, with the same mRNA antigens as described in FIGS. 12A-12B, containing either a secretion signal (FIG. 13a) or both a secretion signal and a transmembrane domain, except for the P028-V7 construct (FIG. 13b), encapsulated in the same LNP. LNP alone was used as negative control. A formulation containing 10 μg of P028-V7 recombinant protein formulated in AlOOH was used as benchmark. The SB assay was performed on a NCTC737 C. acnes strain. LLOQ=lowest limit of quantification.

FIGS. 14A-14B show Surface Binding (SB) titers from pools of sera of mice immunized twice at 4 weeks interval, with the same mRNA antigens as described in FIGS. 12A-12B, containing either a secretion signal (FIG. 14a) or both a secretion signal and a transmembrane domain, except for the P028-V7 construct (FIG. 14b), and encapsulated in the same LNP. LNP alone was used as negative control. A formulation containing 10 μg of P028-V7 recombinant protein formulated in AlOOH was used as a benchmark. The SB assay was performed on KPA171202 C. acnes strain. LLOQ=lowest limit of quantification.

FIGS. 15A-15B show Opsonophagocytic Killing (OPK) titers from pool of sera of mice immunized twice at 4 weeks interval, with the same mRNA antigens as described in FIGS. 12A-12B, containing either a secretion signal (FIG. 15a) or both a secretion signal and a transmembrane domain, except for the P028-V7 construct (FIG. 15b), and encapsulated in the same LNP. LNP alone was used as negative control. A formulation containing 10 μg of P028-V7 recombinant protein formulated in AlOOH was used as a benchmark. The OPK assay was performed on NCTC737 C. acnes strain. LLOQ=lowest limit of quantification.

FIGS. 16A-16B show Opsonophagocytic Killing (OPK) titers from pool of sera of mice immunized twice at 4 weeks interval, with the same mRNA antigens as described in FIGS. 12A-12B, containing either a secretion signal (FIG. 16a) or both a secretion signal and a transmembrane domain, except for the P028-V7 construct (FIG. 16b), and encapsulated in the same LNP. LNP alone was used as negative control. A formulation containing 10 μg of P028-V7 recombinant protein formulated in AlOOH was used as a benchmark. The OPK assay was performed on KPA171202 C. acnes strain. LLOQ=lowest limit of quantification.

FIG. 17 shows Surface Binding (SB) titers from pool of sera, performed on C. acnes strains NCTC737 and KPA171202, complemented by five other strains (as listed in Table 10) to better reflect phylotype proportion found in Acne and based on i) the level of antigen expression and ii) ribotype.

FIG. 18 shows ELISA anti-CAMP2 IgG titers from individual sera of mice immunized twice at 4 weeks interval, with CAMP2 mRNA antigens encapsulated in an LNP. A formulation containing 10 μg of CAMP2 recombinant protein formulated in AlOOH was used as a benchmark. The sera were obtained at day 42 following the first immunization. LNP alone was used as negative control. mRNA synthesis was performed using modified nucleotides. The mRNA content in tested formulation was of 1 μg per dose. LLOQ=lowest limit of quantification.

FIG. 19 shows co-hemolytic neutralizing titers of IgG purified from individual sera of mice immunized twice at 4 weeks interval, with the same mRNA antigens as described in FIG. 18, and encapsulated in the same LNP. A formulation containing 10 μg of CAMP2 recombinant protein formulated in AlOOH was used as a benchmark. The sera were obtained at day 42 following the first immunization. LNP alone was used as negative control. mRNA synthesis was performed using modified nucleotides. The mRNA content in tested formulation was of 1 μg per dose. LLOQ=lowest limit of quantification.

FIG. 20 shows a schematic visualization of the protein domains of DSA1, DSA2 and PITP, and the mRNA constructs that have been synthesized based on these proteins: mRNA H4-V3 (a chimera of DSA1 and DSA2) and mRNA P028-V7 (derived from PITP) as well as the mRNA construct of mRNA #1 CAMP2. The CAMP2 mRNA #1 construct comprised an unmutated CAMP2 sequence, the signal sequence (HA SS) and the transmembrane domain sequence (HA TMB) of Influenza Hemagglutinin (HA, HINI A/Caledonia/20/1999) at N-terminus and C-terminus respectively. The H4-V3 and P028-V7 mRNA constructs comprised the signal sequence (HA SS) of Influenza Hemagglutinin (HA, H1N1 A/Caledonia/20/1999) at N-terminus of the protein coding sequence. CAMP2, H4-V3 and P028-V7 sequences were derived from the protein sequence of C. acnes strain KPA171202. All protein sequences did not contain the native signal sequence of the proteins.

FIG. 21 shows ELISA IgG anti CAMP2 titers and co-hemolytic neutralizing titers of IgG purified from individual sera of mice immunized twice at 4 weeks interval, with the CAMP2 mRNA antigens encapsulated alone or co-encapsulated with the 2 other mRNA antigens (H4-V3 and P028-V7 as described in FIG. 20) in LNPs comprising OF-02, cKK-E10 or GL-HEPES-E3-E12-DS-4-E10 as cationic lipid. A co-mix formulation with the three mRNA antigens separately encapsulated in GL-HEPES-E3-E12-DS-4-E10 LNP was also tested. LOQ=Limit Of Quantification.

FIGS. 22A-22C show ELISA IgG anti-DSA1 (FIG. 22a), anti-DSA2 (FIG. 22b) and anti-PITP (FIG. 22c) titers from individual sera of mice immunized twice at 4 weeks interval with the same mRNA antigens as described in FIG. 20 and encapsulated in the same LNPs as described in FIG. 21.

FIGS. 23A-23B show Surface Binding (SB) titers (FIG. 23a) and Opsonophagocytic Killing (OPK) titers (FIG. 23b), both performed on NCTC737 C. acnes strains, from individual sera of mice immunized twice at 4 weeks interval with the same mRNA antigens as described in FIG. 20 and encapsulated in the same LNPs as described in FIG. 21.

FIGS. 24A-24B show Surface Binding (SB) titers (FIG. 24a) and Opsonophagocytic Killing (OPK) titers (FIG. 24b), both performed on KPA171202 C. acnes strains, from individual sera (SB) or pool of sera (OPK) of mice immunized twice at 4 weeks interval with the same mRNA antigens as described in FIG. 20 and encapsulated in the same LNPs as described in FIG. 21.

FIGS. 25A-25B show Pearson correlations between ELISA IgG anti-DSA1, -DSA2, -PITP, and -CAMP2 titers and co-hemolytic neutralizing titers (CAMP2) or Surface Binding titers (PITP, DSA1 and DSA2), or Opsonophagocytic Killing titers (DSA1 and DSA2) both performed on either KPA171202 (PITP) or NCTC737 (DSA1/DSA2) C. acnes strains, from individual sera of mice immunized twice at 4 weeks interval with the same mRNA antigens as described in FIG. 20 and encapsulated in the same LNPs. r=Pearson correlation factor calculated between each variable.

FIG. 26 shows ELISA IgG anti DSA1 titers from individual sera of mice immunized twice at 4 weeks interval with H4-V3, P028-V7 and CAMP2 recombinant proteins, formulated either alone or in combination [H4-V3 plus P028-V7] or [H4-V3 plus P028-V7 plus CAMP2]. These formulations were done with AlOOH, AF03, SPA14 adjuvants or with no adjuvant. An additional group of mice received 4 immunizations of the bivalent vaccine [H4-V3 plus P028-V7] in AlOOH at 2 weeks interval. The sera were obtained at day 42 following the first immunization. AlOOH and AF03 alone were used as negative control. The H4-V3 and P028-V7 recombinant protein content in tested formulation was of 1 μg or 10 μg of each per dose (in mono-, bi- and tri-valent). The CAMP2 recombinant protein content in tested formulation was 10 μg per dose (in mono- and tri-valent).

FIG. 27 shows ELISA IgG anti PITP titers from individual sera of mice immunized twice at 4 weeks interval with the same recombinant protein antigens as described in FIG. 26 and formulated in the same adjuvants.

FIGS. 28A-28B show ELISA IgG anti CAMP2 titers from individual sera (FIG. 28a) and co-hemolytic neutralizing titers of IgG purified from individual sera (FIG. 28b) of mice immunized twice at 4 weeks interval with the same recombinant protein antigens as described in FIG. 26 and formulated in the same adjuvants.

FIGS. 29A-29B show Surface Binding (SB) titers (FIG. 29a) and Opsonophagocytic Killing (OPK) titers (FIG. 29b), both performed on NCTC737 C. acnes strains, from pool of sera of mice immunized twice at 4 weeks interval with the same recombinant protein antigens as described in FIG. 26 and formulated in the same adjuvants.

FIGS. 30A-30B show Surface Binding (SB) titers (FIG. 30a) and Opsonophagocytic Killing (OPK) titers (FIG. 30b), both performed on KPA171202 C. acnes strains, from individual sera (SB) and pool of sera (OPK) of mice immunized twice at 4 weeks interval with the same recombinant protein antigens as described in FIG. 26 and formulated in the same adjuvants.

FIG. 31 shows a schematic visualization of mRNA constructs coding for chimera proteins that have been designed and synthesized.

FIG. 32 shows Surface Binding (SB) titers performed on NCTC737 C. acnes strain, from individual sera of mice immunized twice at 4 weeks interval with the mRNA antigens described in FIG. 31 (“H4-V3”, “H4-V3-lowglyc”, “P028-V7-F16”, “P028-V7-C₁₂” and “P028-V7-C₁₂lowglyc”) and with the mRNA constructs coding for P028-V7. These mRNA constructs were encapsulated in the LNP GL-HEPES-E3-E12-DS-4-E10. The sera were obtained at day 42 following the first immunization. LNP alone was used as negative control. mRNA synthesis was performed using modified nucleotides. The mRNA content in tested formulation was of 1 μg and 5 μg per dose.

FIG. 33 shows Surface Binding (SB) titers performed on KPA171202 C. acnes strain, from individual sera of mice immunized twice at 4 weeks interval with the same mRNA antigens as described in FIG. 32, and encapsulated in the same LNP. The sera were obtained at day 42 following the first immunization. LNP alone was used as negative control. mRNA synthesis was performed using modified nucleotides. The mRNA content in tested formulation was of 1 μg and 5 μg per dose.

FIGS. 34A-34C show ELISA IgG anti-DSA1 (FIG. 34a), anti-DSA2 (FIG. 34b) and anti-PITP (FIG. 34c) titers from individual sera of mice immunized twice at 4 weeks interval with the same mRNA antigens as described in FIG. 32 and encapsulated in the same LNPs. The sera were obtained at day 42 following the first immunization. LNP alone was used as negative control. mRNA synthesis was performed using modified nucleotides. The mRNA content in tested formulation was of 1 μg per dose.

FIGS. 35A-35C show ELISA IgG anti-DSA1 (FIG. 35a), anti-DSA2 (FIG. 35b) and anti-PITP (FIG. 35c) titers from individual sera of mice immunized twice at 4 weeks interval with the same mRNA antigens as described in FIG. 32 and encapsulated in the same LNPs. The sera were obtained at day 42 following the first immunization. LNP alone was used as negative control. mRNA synthesis was performed using modified nucleotides. The mRNA content in tested formulation was of 5 μg per dose.

FIG. 36 shows Opsonophagocytic Killing (OPK) titers performed on KPA171202 C. acnes strain, from serum pools of mice immunized twice at 4 weeks interval with the same mRNA antigens as described in FIG. 32, and encapsulated in the same LNP. The sera were obtained at day 42 following the first immunization. LNP alone was used as negative control. mRNA synthesis was performed using modified nucleotides. The mRNA content in tested formulation was of 1 μg and 5 μg per dose. Results illustrate the means of 2 independent experiments. LLOQ=lowest limit of quantification.

FIG. 37 shows Opsonophagocytic Killing (OPK) titers performed on KPA171202 C. acnes strain, from individual sera of mice immunized twice at 4 weeks interval with the same mRNA antigens as described in FIG. 32, and encapsulated in the same LNP. The graph illustrated the results for P028-V7-F16, P028-V7-C12 and P028-V7 mRNA constructs. The sera were obtained at day 42 following the first immunization. LNP alone was used as negative control. mRNA synthesis was performed using modified nucleotides. The mRNA content in tested formulation was of 1 μg and 5 μg per dose. LLOQ=lowest limit of quantification.

FIG. 38 shows Opsonophagocytic Killing (OPK) titers performed on NCTC737 C. acnes strain, from serum pools of mice immunized twice at 4 weeks interval with the same mRNA antigens as described in FIG. 32, and encapsulated in the same LNP. The sera were obtained at day 42 following the first immunization. LNP alone was used as negative control. mRNA synthesis was performed using modified nucleotides. The mRNA content in tested formulation was of 1 μg and 5 μg per dose. Results illustrate the means of 2 independent experiments. LLOQ=lowest limit of quantification.

FIG. 39 shows Opsonophagocytic Killing (OPK) titers performed on NCTC737 C. acnes strain, from individual sera of mice immunized twice at 4 weeks interval with the same mRNA antigens as described in FIG. 32, and encapsulated in the same LNP. The graph illustrated the results for P028-V7-C12, P028-V7-C12-lowglyc, H4-V3 and H4-V3-lowglyc mRNA constructs. The sera were obtained at day 42 following the first immunization. LNP alone was used as negative control. mRNA synthesis was performed using modified nucleotides. The mRNA content in tested formulation was of 1 μg and 5 μg per dose.

FIGS. 40A-40C show ELISA anti-DsA1 (FIG. 40a), anti-DsA2 (FIG. 40b) and anti-PITP (FIG. 40c) IgG titers of individual sera (day 42) of mice immunised twice at 4 weeks interval with an mRNA construct encoding a chimeric DsA1/DsA2/PITP protein or a combination of two mRNAs encoding H4-V3 and P028-V7 proteins, respectively, at various doses, and encapsulated in a LNP containing GL-HEPES-E3-E12-DS-4-E10 as cationic lipid. LNP alone served as a negative control. LLOQ=lowest limit of quantification.

FIGS. 41A-41B show Surface Binding (SB) titers from individual sera (day 42) of mice immunised twice at 4 weeks interval with the same mRNA antigens as described in FIGS. 40A-40C and encapsulated in the same LNP. LNP alone served as a negative control. The SB assay was performed on NCTC737 C. acnes strain (FIG. 41a) or KPA171202 C. acnes strain (FIG. 41b).

FIGS. 42A-42B show Opsonophagocytic Killing (OPK) titers from individual sera (day 42) of mice immunised twice at 4 weeks interval with the same mRNA antigens as described in FIGS. 40A-40C and encapsulated in the same LNP. LNP alone served as a negative control. The OPK assay was performed on NCTC737 C. acnes strain (FIG. 42a) or KPA171202 C. acnes strain (FIG. 42b).

FIG. 43 shows a table summarizing the Western blot analysis of protein expression and localization from CAMP2 and PITP mRNA constructs containing a SS from glycoprotein derived from Influenza (subtypes A or B), Rabies, Varicella (VZV), or Ebola viruses, with or without their respective glycoprotein TMB domain post transfection in HEK Expi293F cells.

FIGS. 44A-44C show alignment of 27 different CAMP2 polypeptide sequences identified from the analysis of 430 sequences of CAMP2 from naturally occurring C. acnes strains (including KPA171202 and ATCC6919). Sequence variation between those CAMP2 polypeptides is concentrated at 34 residues identified across the 239 amino acid-long sequence (excluding the secretion signal peptide sequence). FIGS. 44a, b and c display different (overlapping) segments of the alignment: residues 1-101 (a), 99-201 (b) and 199-267 (c)-residue numbering including the secretion signal peptide sequence.

FIG. 45 shows a schematic visualization of the “quadruple chimera” mRNA constructs that have been designed, synthesized and assessed experimentally.

FIGS. 46A-46B shows levels of protein expression in supernatant (FIG. 46a) and cell lysate (FIG. 46b), as measured by HPLC-MS, after in vitro cell transfection of HEK Expi293F cells with “quadruple chimera” mRNA constructs (construct designs shown in FIG. 45) and other mRNA constructs used as controls.

FIGS. 47A-47D show ELISA anti-DsA1 (FIG. 47a), anti-DsA2 (FIG. 47b), anti-PITP (FIG. 47c) and anti-CAMP2 (FIG. 47d) IgG titers of sera (day 42 after first immunization) of mice immunized twice at 4 weeks interval with “quadruple chimera” mRNA constructs (construct designs shown in FIG. 45), in comparison with a combination of an mRNA construct encoding CAMP2 expressed at the cell membrane and an mRNA construct encoding the H4-V3-f-PRO-028-V7-F16 triple chimera. Two doses were assessed (1 μg and 5 μg). mRNA constructs were encapsulated in an LNP comprising GL-HEPES-E3-E12-DS-4-E10 as cationic lipid. LNP alone served as a negative control. EU=ELISA unit.

FIGS. 48A-48B show Opsonophagocytic Killing (OPK) titers from individual sera (day 42 after first immunization) of mice immunized twice at 4 weeks interval with the same mRNA constructs, at the same doses and encapsulated in the same LNP, as described in FIGS. 47A-47D. LNP alone served as a negative control. The OPK assay was performed on C. acnes strain NCTC737 (FIG. 48a) or C. acnes strain KPA171202 (FIG. 48b).

FIGS. 49A-49B show Surface Binding (SB) titers from individual sera (day 42 after first immunization) of mice immunized twice at 4 weeks interval with the same mRNA constructs, at the same doses and encapsulated in the same LNP, as described in FIGS. 47A-47D. LNP alone served as a negative control. The SB assay was performed on C. acnes strain NCTC737 (FIG. 49a) or C. acnes strain KPA171202 (FIG. 49b). LLOQ=lowest limit of quantification

FIG. 50 shows co-hemolytic neutralizing titers of IgG purified from individual sera (day 42 after first immunization) of mice immunized twice at 4 weeks interval with the same mRNA constructs, at the same doses and encapsulated in the same LNP, as described in FIGS. 47A-47D. LNP alone served as a negative control. LLOQ=lowest limit of quantification

### MODES FOR CARRYING OUT THE INVENTION

### Example 1—Main Methods

### Enzyme-Linked Immunosorbent Assays (ELISA)

An ELISA method was used to detect and quantify the IgG immune response elicited by various antigens (e.g. various constructs based on DsA1, DsA2, PITP or CAMP2) following administration in mice.

The specific IgG titers were measured from individual sera using an ELISA method in a 384-well plate on a Hamilton automated platform, or manually. Briefly, 384-well micro-plates were coated with the recombinant protein of interest (e.g. DsA1, DsA2, PITP, CAMP2), at protein concentrations ranging from 0.25 to 2 μg/mL in PBS 1×, and kept overnight at +4° C. Coating solution was removed and washed with buffer 1 (PBS 1×/Tween20 0.1%). Unspecific binding was prevented by incubating the coated plates with a saturation buffer containing either 2% BSA or 1% skimmed milk in PBS1× during 90 min at room temperature (RT). Plates were emptied, then a 2-fold serial dilution of the serum samples (containing the specific IgG antibodies to be measured) were added to the plates. The plates were incubated for 90 min at RT and then washed with buffer 1. The formed antigen-antibodies complexes were subsequently revealed by adding an anti-species secondary antibody (Goat Anti-Mouse IgG; JACKSON; 115-036-062 or Goat Anti-Rabbit IgG; SIGMA; A0545) conjugated with horse radish peroxidase (HRP). After 90 min incubation at RT, the plates were washed with buffer 1. The reaction was developed by adding a substrate of peroxidase (H₂O₂) combined with a chromogen (TMB solution), inducing the hydrolysis of the substrate and developing a yellow coloration. The hydrolysis reaction was chemically stopped after 30 min at RT by the addition of HCl (IN (normality)), and Optical Density (OD) was measured at 450-650 nm on a spectrophotometer (Synergy HTX, Biotek). The difference between the OD at 450 nm and the OD at 650 nm is directly proportional to the amount of specific IgG antibodies bound to the recombinant protein of interest (antigen). The IgG titers of a serum sample were calculated either directly by expressing a titer that is the inverse of the dilution corresponding to an OD equal to 1, or relative to a reference serum of the same animal species applied to each plate. The IgG titers (Arbitrary units) were calculated using Softmax Pro software.

### Surface Binding Assays (SBA)

Following administration of various antigens (e.g. various constructs based on DsA1, DsA2, PITP or CAMP2) in mice, Surface Binding Assays (SBA) using a fluorescence cytometer were used to determine the binding efficiency of serum antibodies to the cell surface of live Cutibacterium acnes (C. acnes) bacteria. In particular, the ability to bind the cell surface of C. acnes strains from different genetic types was used to assess cross-reactivity of the serum antibodies.

Briefly, bacterial suspensions of C. acnes strains (e.g. C. acnes types IA1 (NCTC737) and IB (KPA171202)) were precultured in Brucella Blood Agar with Hemin and Vitamin K1 (BD) for 48- to 72-hours anaerobically and inoculated in Thioglycollate broth at OD600 nm of 0.15 for 16- to 18-hours at 37° C. in anaerobic atmosphere. The precultures were then diluted in Thioglycollate with or without the addition of 750 μg/mL deferoxamine at OD600 nm of 0.15 and incubated 24-hours at 37° C. under anaerobic conditions.

Bacteria were washed and resuspended in HBSS buffer containing 2% Bovine Serum Albumin (BSA) to a final concentration of 4×10⁶ Colony Forming Units (CFU)/mL. Heat inactivated serum samples and the assay control serum were brought to RT and diluted in HBSS 2% BSA. 50 μL of a 2-fold serial dilution of the serum samples and 50 μL of bacteria suspension were deposited in a 96-deepwell plate, and incubated for 30 min at RT. To reveal antibodies (contained in the serum) bound to bacteria, reaction plates were washed twice and 100 μL of secondary anti-mouse/rabbit IgG antibody conjugated to Alexa Fluor 488 (Alexa Fluor 488 F(ab′) 2 fragment of goat anti mouse IgG/life technology #A1101) (diluted 1:500 in HBSS containing 0.5% BSA) were added, and incubated for 30 min at RT. 50 μL of SYTO60 (diluted 1:500 in HBSS) were added to the reaction and incubated for 15 min at RT, in order to stain bacteria. Reaction plates were washed twice and bacteria were resuspended in 150 μL HBSS for the acquisition of median fluorescence intensity (MFI) with a flow cytometer (NovoCyte, Agilent). The MFI is a correlate of the amount of surface-bound antibodies.

### Opsonophagocytic Killing (OPK) Assays

Opsonophagocytic killing (OPK) assays may be used to determine the ability of antibodies to induce opsonisation of C. acnes bacteria by mobilizing immune cells against the bacteria, leading to a reduction in the bacterial cell numbers. OPK assays were used to test serum antibodies elicited by various antigens (e.g. various constructs based on DsA1, DsA2, PITP or CAMP2) following their administration in mice.

The OPK assay involves co-culture of HL-60 cell line (a human promyelocytic leukemia cell line), differentiated into granulocyte-like human cells with live C. acnes bacteria pre-incubated with serum antibodies.

Briefly, HL-60 cells (at a concentration of 5×10⁵ cells/mL) were differentiated in cell differentiation medium containing dimethylformamide (DMF, Sigma), i.e. RPMI medium (RPMI medium 1640 1×+Glutamax (Gibco)) containing 10% heat inactivated FCS and 0.8% DMF, for 3 days at 37° C. After washing, the cell number was adjusted to a final concentration of 5,28×10⁶ cells/ml in RPMI medium containing 10% FCS and 20 mM glucose.

Bacterial suspensions of C. acnes strains (e.g. C. acnes multilocus sequence types IA1 (NCTC737) and IB (KPA171202)) were cultured in thioglycolate broth at 37° C. in anaerobic atmosphere to reach an OD600 nm in the range 0.3 to 1.7. Bacteria were washed and resuspended in HBSS buffer containing 2% BSA, to a final concentration of 6×10⁴ CFU/mL (corresponding to a final number of 1500 bacteria per well during the reaction) and incubated at room temperature for at least 30 minutes as saturation step.

Heat inactivated serum samples and the assay control serum were brought to RT. If required, predilution was performed in RPMI medium containing 10% FCS and 20 mM glucose. 50 μL of a 2-fold serial dilution of the serum samples were deposited in a 96-well reaction plate.

HL-60 cells were mixed with the bacteria at a dilution of 1:6 (in order to have a ratio a 440 HLA-60 cells for 1 bacterium), and 150 μL of the HL-60 cells/bacteria suspension were added in the wells containing the diluted serum samples. After a two-day reaction, 100 μL of each reaction were plated on Brucella blood agar at three different dilutions. E.g., 40 μL of diluted reaction mix (1/50 for the NCTC737 strain and 1/200 for the KPA171202 strain) were deposited on BBA. After anaerobic incubation of the plates at 37° C. for a minimum of 70 hours, the colonies on the plates were counted with a colony counter (AES Laboratories); a picture of the agar plate with a microcamera, and image analysis using the Cybel software was performed to count colonies. The number of CFU in experimental conditions (with differentiated HL-60) was compared to the CFU of the corresponding negative controls. The OPK activity was expressed as the titer corresponding to the reciprocal serum dilution that induces 50% decrease in the viable bacterial counts (CFU) in comparison with negative control sera (K50 titer), as determined using a four-parameter logistic regression model.

### Co-Hemolysis Neutralisation Assay

This assay was used to measure serological titres of neutralizing recombinant C. acnes CAMP2 polypeptide (rCAMP2)-specific antibodies.

Briefly, IgG from mouse sera were purified with Protein G High Performance Spintrap kit (Cytiva), according to manufacturer's instructions.

Sheep red blood cells (SRBC) were prepared from whole blood suspended in Alsever anticoagulant (50% v/v), washed three times with cold PBS and resuspended in PBS by adding a volume equivalent to 10 times that of the pellet. SRBC were subsequently treated with sphingomyelinase (0.025 U/mL final concentration) for 30 minutes at 37° C., then washed three times with PBS at RT. SRBC concentration was adjusted to 8·10⁷ cells/mL.

25 μL of a 2-fold serial dilution of purified IgG and 25 μL of rCAMP2 at 10 μg/mL were deposited in a 96-well reaction plate, which were incubated at 37° C. for 1 hour. Then, 50 μL of SRBC were added, and the final reaction was incubated for 2 hours at 37° C. The pellet area in each well were recorded using a Multimode Imagine plate reader.

The presence of antibodies that neutralize rCAMP2 biological activity resulted in co-hemolysis inhibition and therefore allowed the deposition of intact red blood cells at the bottom of the well, forming a pellet. Neutralising titers were defined as the reciprocal dilution that corresponds to 75% of the treated negative control pellet area, referred to Hemolysis Effect 75 (HE75). The determination of the titer corresponding to HE75 was based on a four-parameter logistic regression model.

### Example 2-Comparison of mRNA Platform Versus Protein Platform, for CAMP2, H4-V3 and P028-V7 Antigens

The aim of this example was to compare formulations containing mRNA encoding C. acnes antigens relative to formulations containing C. acnes polypeptides for their ability to induce a functional immune response. The tested antigens included C. acnes CAMP2 polypeptides, a chimeric C. acnes DsA1/DsA2 polypeptide (H4-V3) and C. acnes PITP polypeptide (P028-V7).

Preparation of mRNA-LNPs

Each mRNA comprised a cap1, a 5′UTR from CMV given
by the sequence:
(SEQ ID NO: 265)
GGACAGAUCGCCUGGAGACGCCAUCCACGCUGUUUUGACCUCCAUAGAAG
ACACCGGGACCGAUCCAGCCUCCGCGGCCGGGAACGGUGCAUUGGAACGC
GGAUUCCCCGUGCCAAGAGUGACUCACCGUCCUUGACACG,
a 3′ UTR from hGH given by the sequence:
(SEQ ID NO : 266)
CGGGUGGCAUCCCUGUGACCCCUCCCCAGUGCCUCUCCUGGCCCUGGAA
GUUGCCACUCCAGUGCCCACCAGCCUUGUCCUAAUAAAAUUAAGUUGCA
UC,

and a polyA tail. Each mRNA was encapsulated in lipid nanoparticles (LNPs) composed of four lipids: ionizable (cationic) lipid/DOPE/cholesterol/DMG-PEG, at the molar ratio of 40:30:28.5:1.5. In this example, ckk-E10 was used as the ionizable lipid for mRNAs encoding C. acnes CAMP2 polypeptides and GL-HEPES-E3-E12-DS-4-E10 was used as the ionizable lipid for mRNA encoding H4-V3 and mRNA encoding P028-V7.

LNPs were prepared using a T-mix process. Briefly, lipids were dissolved in ethanol (EtOH) at a final concentration of 9.331 mg/ml and mRNA solution was prepared in Citrate Buffer Saline (CBS at 1 mM citrate pH 4.5 and 150 mM sodium chloride). 3 mL of the lipid solution and 12 mL of the mRNA solution were loaded respectively in a 5 mL and 20 ml syringe. Using a T-mix system 3/32″ and pumps, mRNA solution and lipids were mixed at a total flow rate of 250 mL/min (flow rate at 50m L/min for the lipids and flow rate of 200 mL/min for the mRNA). The mixture was then treated by buffer exchange. First, a dialysis in water: EtOH 80:20 was performed 2 h at room temperature (RT), then a second dialysis in water was performed overnight at +4° C. The last step was a concentration to 1 mg/mL mRNA and a buffer exchange in Amicon at +4° C. with trehalose 10%. LNPs were then sterile filtered and stored at −80° C.

### Immunisation of Mice

OF-1 outbred mice (6-week old at day 0) were immunized with the mRNA or protein antigen (at doses ranging between 0.2 and 10 μg for mRNA CAMP2, and at 1 and 5 μg doses for mRNAs H4-V3 and P028-V7, and at 10 μg for all proteins), twice, 3 or 4 weeks apart, through intra-muscular (IM) route (50 μL in quadriceps muscle hind leg, with alternance of the right and left leg between both injections). LNP alone served as negative controls for the mRNA immunisations. A final bleeding was performed 7 or 14 days after the last injection, and the sera obtained were tested in a co-hemolysis neutralisation assay (as described in Example 1), in a Surface Binding assay (as described in Example 1) and/or in an OPK assay (as described in Example 1).

Immunisation with C. acnes CAMP2 Antigens Elicits Antibodies which Decrease the Co-Hemolytic Activity of CAMP 2

C. acnes CAMP2 antigens were evaluated for their ability to elicit antibodies which reduce the inflammatory activity of C. acnes CAMP2, using a co-hemolysis neutralisation assay.

The following antigens were tested:
Two different codon-optimized mRNAs, both encoding a wild-type C. acnes CAMP2 polypeptide with a N-terminal HA SS (SEQ ID NO: 2): “ssHA-CAMP2_WT_GA” (SEQ ID NO: 87) and “ssHA-CAMP2_WT_GS” (SEQ ID NO: 88)
Two different codon-optimized mRNAs, both encoding a C. acnes CAMP2 polypeptide with mutations at N-Glycosylation sites and with a N-terminal HA SS (SEQ ID NO: 10): “ssHA-CAMP2_G-_GA” (SEQ ID NO: 95) and “ssHA-CAMP2_G-_GS” (SEQ ID NO: 96)
Recombinant C. acnes CAMP2 polypeptide of SEQ ID NO: 1

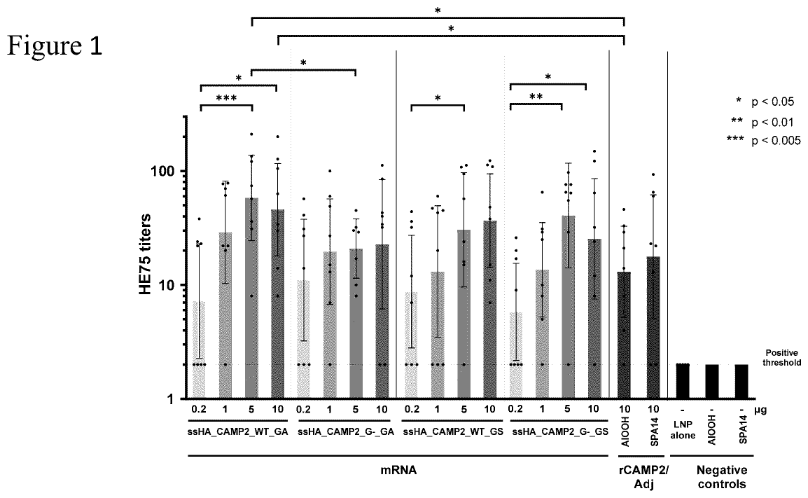

mRNA sequences were designed for coding secreted form of CAMP2. Each mRNA sequence was optimized using two different algorithms, and mRNA synthesis was performed using unmodified nucleotides. Each mRNA antigen construct (encapsulated in a LNP using ckk-E10 as the ionizable lipid) was tested at doses ranging from 0.2 to 10 μg per mouse. Recombinant C. acnes CAMP2 polypeptide of SEQ ID NO: 1 was formulated in either aluminium hydroxide (AlOOH) or SPA 14 and tested at 10 μg per mouse. Mice were immunized twice at 3 weeks interval. The sera were obtained at day 35 following the first immunization and IgG were purified from the sera. As shown in FIG. 1, all mRNAs containing formulations induced neutralizing titers equivalent or superior to formulations with recombinant CAMP2. The optimal dose for mRNA containing formulation was 5 μg.

Evaluation of Chimeric C. acnes DsA1/DsA2 Antigens

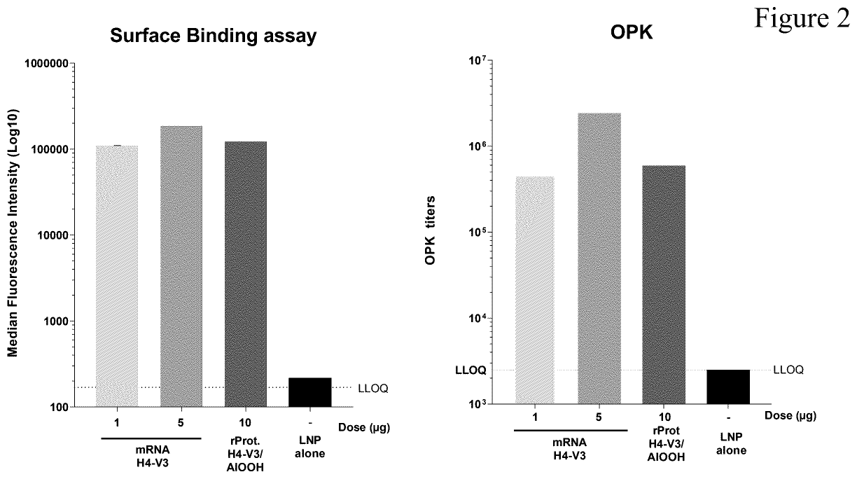

An mRNA encoding a chimeric C. acnes DsA1/DsA2 polypeptide, H4-V3 (SEQ ID NO: 28, having a nucleotide sequence corresponding to SEQ ID NO: 113) was tested for its ability to elicit opsonising antibodies. mRNA sequences were designed for coding secreted form of H4-V3. The mRNA was synthesised using modified nucleotides (in which all uridine nucleosides were replaced with 1-methyl pseudo uridine). The mRNA (encapsulated in a LNP using GL-HEPES-E3-E12-DS-4-E10 as the ionizable lipid) was tested at a dose of 1 and 5 μg per mouse. Recombinant H4-V3 polypeptide of SEQ ID NO: 29 (formulated in AlOOH adjuvant) was tested at 10 μg per mouse. Mice were immunized twice at 4 weeks interval. The sera were obtained at day 42 following the first immunization, and Surface Binding (SB) and Opsonophagocytic Killing (OPK) were performed from pools of sera (OPK) on NCTC737 C. acnes strain (IA1, RT1). This strain expressed DsA1, DsA2 and PITP. As shown in FIG. 2, the mRNA platform performed at least as well as the protein platform when a DsA1/DsA2 chimera (H4-V3) was used as antigen, as evidenced by a surface binding assay and an OPK assay.

Evaluation of C. acnes PITP Antigens

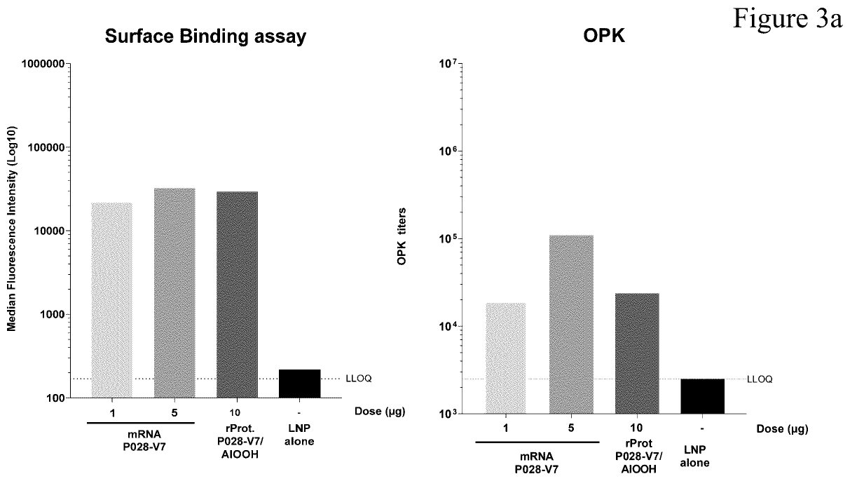

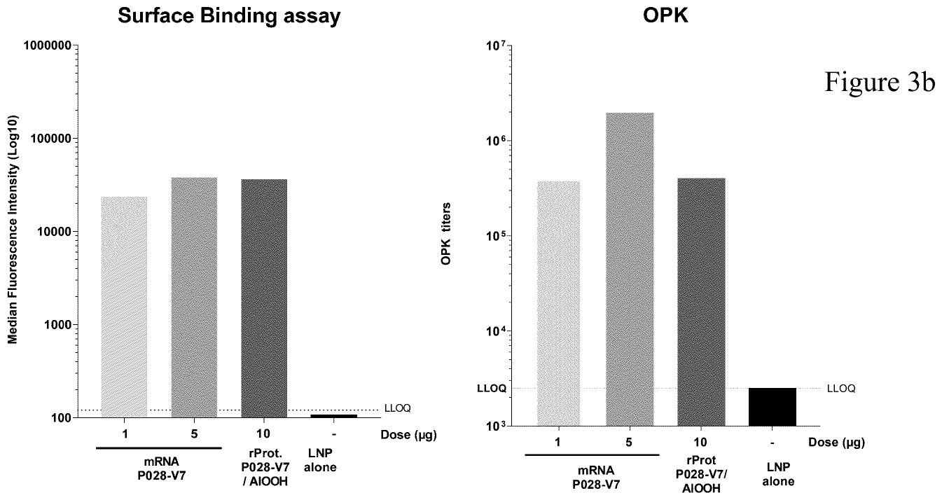

An mRNA encoding a C. acnes PITP polypeptide, P028-V7 (SEQ ID NO: 31, having a nucleotide sequence corresponding to SEQ ID NO: 115) was tested for its ability to elicit opsonising antibodies. mRNA sequences were designed for coding secreted form of P028-V7. The mRNA was synthesised using modified nucleotides (in which all uridine nucleosides were replaced with 1-methyl pseudo uridine). The mRNA (encapsulated in a LNP using GL-HEPES-E3-E12-DS-4-E10 as the ionizable lipid) was tested at a dose of 1 and 5 μg per mouse. Recombinant P028-V7 polypeptide of SEQ ID NO: 32 (formulated in AlOOH adjuvant) was tested at 10 μg per mouse. Mice were immunized twice at 4 weeks interval. The sera were obtained at day 42 following the first immunization, and Surface Binding (SB) and Opsonophagocytic Killing (OPK) experiments were performed from pools of sera on NCTC737 and KPA171202 C. acnes strains. The mRNA platform performed at least as well as the protein platform when a PITP variant (P028-V7) was used as antigen, as evidenced by a surface binding assay and an OPK assay, using a NCTC737 strain of C. acnes (IA1, RT1), or a KPA171202 strain of C. acnes, as shown in FIGS. 3a and 3b respectively. The NCTC737 strain expressed DsA1, DsA2 and PITP. The KPA171202 strain expressed PITP, but not DsA1 or DsA2.

### Example 3-Evaluation of CAMP2 mRNA Antigens

The aim of this example was to assess a range of C. acnes CAMP2 antigen constructs for their ability to induce a functional immune response, as assessed using co-hemolytic neutralising assays and ELISA assays.

Design of C. acnes CAMP2 Polypeptide Constructs

All C. acnes CAMP2 polypeptide sequences were derived from the C. acnes strain KPA171202 CAMP2 polypeptide sequence lacking a native signal sequence.

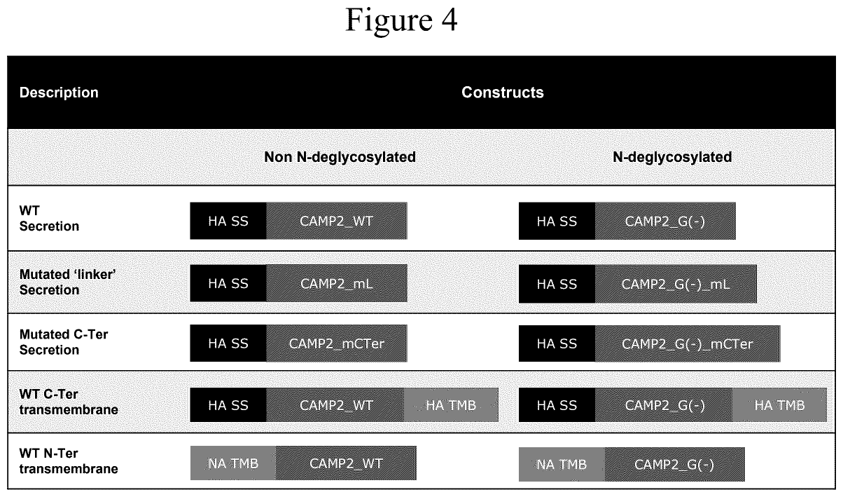

Six mRNA constructs encoding secreted C. acnes CAMP2 polypeptide constructs were designed as depicted in the top three lines in the Table of FIG. 4. Each construct comprised a N-terminal secretion peptide signal sequence of Influenza Hemagglutinin (HA, H1N1 A/Caledonia/20/1999) (HA SS) in order to target the encoded protein for secretion. The non-N-deglycosylated constructs (LHS) include:
a native C. acnes CAMP2 polypeptide (“HA SS CAMP2_WT”) (mRNA according to SEQ ID NO: 89),
a C. acnes CAMP2 polypeptide with amino acid mutations in the linker moiety (“HA SS CAMP2_mL”) (mRNA according to SEQ ID NO: 98)
a C. acnes CAMP2 polypeptide with amino acid mutations within its C-terminal domain (“HA SS CAMP2_mCTer”) (mRNA according to SEQ ID NO: 100)

For each of the three non-N-deglycosylated constructs, a N-deglycosylated counterpart construct was designed with mutations in a N-glycosylation site (a substitution of Asn166 by Ser) (RHS):
a C. acnes CAMP2 polypeptide with a N166S mutation (“HA SS CAMP2_G(−)”) (mRNA according to SEQ ID NO: 97)
a C. acnes CAMP2 polypeptide with amino acid mutations in the linker moiety and with a N166S mutation (“HA SS CAMP2_G(−)_mL”) (mRNA according to SEQ ID NO: 99)
a C. acnes CAMP2 polypeptide with amino acid mutations within its C-terminal domain and with a N166S mutation (“HA SS CAMP2_G(−) mCTer”) (mRNA according to SEQ ID NO: 101)

Additionally, four mRNA constructs encoding membrane-anchored C. acnes CAMP2 polypeptide constructs were designed as depicted in the two bottom lines in the Table of FIG. 4. The non-N-deglycosylated constructs (LHS) include:
a native C. acnes CAMP2 polypeptide targeted to the membrane via a C-terminal transmembrane domain sequence (HA TMB) of Influenza Hemagglutinin (HA, H1N1 A/Caledonia/20/1999) (“HA SS CAMP2_WT HA TMB”) (mRNA according to SEQ ID NO: 90)
a native C. acnes CAMP2 polypeptide targeted to the membrane via a N-terminal transmembrane domain sequence (NA TMB) of Neuraminidase (HA, H1N1 A/Caledonia/20/1999) (“NA TMB CAMP2_WT”) (mRNA according to SEQ ID NO: 92)

For each of the two Non-N-deglycosylated constructs, a N-deglycosylated counterpart construct was designed with a mutation in a N-glycosylation site (a substitution of Asn166 by Ser) (RHS):
a C. acnes CAMP2 polypeptide with a N166S mutation targeted to the membrane via a C-terminal transmembrane domain sequence (HA TMB) of Influenza Hemagglutinin (HA, H1N1 A/Caledonia/20/1999) (“HA SS CAMP2_G(−) HA TMB”) (mRNA according to SEQ ID NO: 94)
a C. acnes CAMP2 polypeptide with a N166S mutation targeted to the membrane via a N-terminal transmembrane domain sequence (NA TMB) of Neuraminidase (HA, H1N1 A/Caledonia/20/1999) (“NA TMB CAMP2_G(−)”) (mRNA according to SEQ ID NO: 93)

### Generation of Polyclonal Antibodies

The anti-CAMP2 rabbit polyclonal antibodies used in this example were provided in the form of a pool of 3 rabbit polyclonal sera. Female KBL NZ white rabbit received three immunizations of recombinant C. acnes CAMP2 polypeptide (SEQ ID NO: 1) formulated in SPA14 and given by the IM route at week, week 3 and week 6. The final bleedings were performed on day 56. The sera were obtained by collecting the supernatant after clotting and centrifugation of the blood. Aliquots of the sera were stored at ≤−20° C.

### Generation of Monoclonal Antibodies

The monoclonal antibodies G05, G03 and D07 were identified by phage display using a humanized synthetic VHH library. Phages were selected against recombinant C. acnes CAMP2 polypeptide (SEQ ID NO: 1) expressed in a E. coli-based cell free expression system. After an ELISA screening, selected clones were sequenced, reformatted into minibodies (VHH-human Fc), produced in mammalian cell and purified. Then, minibodies were characterized by BLI to verify and characterise the affinity and the epitope within recombinant C. acnes CAMP2 polypeptide. The functional activity was characterised using a co-hemolysis neutralisation assay (as described in Example 1). G05, G03 and D07 clones were selected as the best-performing clones. G05, D07 and G03 (which targets the same epitope as D07) were found to be functional mAbs, since they were shown to decrease the co-hemolytic activity of CAMP2.

Characterisation of the Expression Products of C. acnes CAMP2 Constructs in HEK Cells

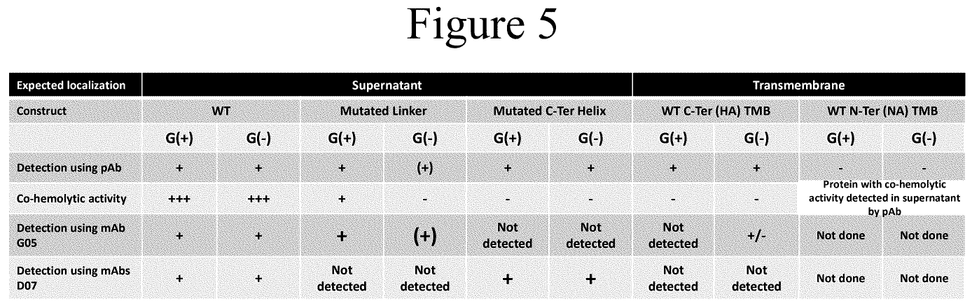

Next, the mRNA constructs encoding C. acnes CAMP2 polypeptide depicted in FIG. 4 were transfected into HEK cells (in vitro cell transfection). Protein expression was monitored in the cell compartment where expression was expected (i.e., in the cell supernatant for constructs designed to be secreted and in the membrane for constructs designed to be membrane-anchored). The expression compartment of C. acnes CAMP2 polypeptides were tested by Western-Blot using anti-CAMP2 rabbit polyclonal antibodies (pAb) and monoclonal antibodies (G05 and D07 mAbs). As shown in FIG. 5, the C. acnes CAMP2 polypeptides designed to be secreted were detected in the supernatant for all constructs as expected. The C. acnes CAMP2 polypeptides designed to be membrane-anchored using a C-terminal HA TMB were detected at the cell membrane for all constructs as expected. In contrast, CAMP2 C. acnes CAMP2 polypeptides designed to be membrane-anchored using a N-terminal NA TMB were detected in the supernatant, indicating that this construct did not localise to the membrane.

Next, the expression products of the mRNA constructs encoding C. acnes CAMP2 polypeptides depicted in FIG. 4 were evaluated for their co-hemolytic activity, with the aim of further identifying assessing whether anchoring the protein into the membrane and mutations in C. acnes CAMP2 polypeptides affect the co-hemolytic activity of the proteins. As shown in the Table in FIG. 5, HA SS CAMP2_WT (“WT G(+)”) and its N-deglycosylated counterpart HA SS CAMP2_G(−) (“WT G(−)”) showed strong co-hemolytic activity. C. acnes CAMP2 polypeptide successfully anchored at the membrane, HA SS CAMP2_WT HA TMB (“WT C-Ter (HA) TMB G(+)”) and HA SS CAMP2_G(−) HA TMB (“WT C-Ter (HA) TMB G(−)”) showed a reduced or abolished co-hemolytic activity. Mutations in the C-Terminal helix (“Mutated C-ter Helix” constructs) or the linker (“Mutated Linker” constructs) either reduced or abolished the co-hemolytic activity of CAMP2. The NA TMB CAMP2_WT and NA TMB CAMP2_G(−) (“WTN-Ter (NA) TMB”) constructs (which were designed to be anchored at the membrane but were instead detected in the supernatant), gave rise to co-hemolytic activity in the supernatant. Collectively, these observations indicated that the linker and C-terminal helix contained or contributed to functional domains of the protein.

The expression products of the mRNA constructs encoding C. acnes CAMP2 polypeptides were then further probed using monoclonal antibodies for the presence of functional epitopes. As shown in FIGS. 5, G05 and D07 are functional mAbs, as they are able to decrease the co-hemolytic activity of CAMP2. Therefore, detection of a given C. acnes CAMP2 polypeptide with these mAbs was interpreted as an indication that the polypeptide contained functional epitopes. All mAbs detected HA SS CAMP2 WT and its N-deglycosylated counterpart HA SS CAMP2_G(−). However, detection with mAb G05 was abolished with CAMP2 constructs with a mutated linker, while mAb D07 was abolished with CAMP2 constructs with mutated C-terminal helix. Collectively, these observations indicated that the linker and C-terminal helix contained functional epitopes.

Immunisation with C. acnes CAMP2 Antigens Elicits Antibodies that Decrease the Co-Hemolytic Activity of CAMP2

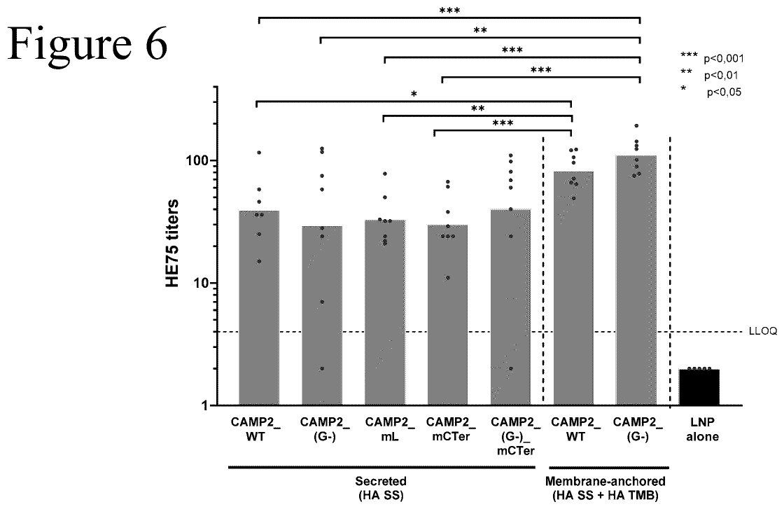

Seven mRNA constructs encoding C. acnes CAMP2 polypeptides over the 10 constructs described in FIG. 4 were synthesised using modified nucleotides (in which all uridine nucleosides were replaced with 1-methyl pseudo uridine) and encapsulated in an LNP comprising GL-HEPES-E3-E12-DS-4-E10 as cationic lipid. LNP alone served as a negative control. These formulations were used to immunise mice twice at 4 weeks interval, each time at a dose of 5 μg per mouse. Mice were immunized twice at 4 weeks interval. The sera were obtained at day 42 following the first immunization, and IgG was purified from the sera. As shown in FIG. 6, membrane-anchored C. acnes CAMP2 polypeptides (“HA SS CAMP2_WT HA TMB” and “HA SS CAMP2_G(−) HA TMB”) both showed significantly higher homogenous neutralizing titers relative to the other tested constructs. “HA SS CAMP2 WT HA TMB” and “HA SS CAMP2_G(−) HA TMB” performed comparably.

Immunisation with C. acnes CAMP2 Antigens Elicits an Anti-CAMP2 IgG Response

mRNA constructs encoding C. acnes CAMP2 polypeptides (as described in FIG. 4) were synthesised using modified nucleotides (in which all uridine nucleosides were replaced with 1-methyl pseudo uridine) and encapsulated in an LNP comprising GL-HEPES-E3-E12-DS-4-E10 as cationic lipid. LNP alone served as a negative control. These formulations were used to immunise mice twice at 4 weeks interval, each time at a dose of 5 μg per mice. The sera were obtained at day 42 following the first immunization, and IgG was purified from the sera.

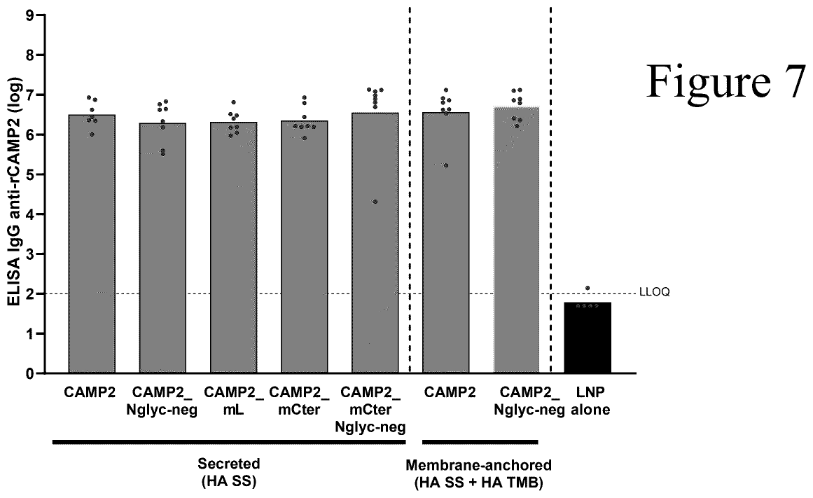

The titers of anti C. acnes CAMP2 IgG as measured by ELISA are shown in FIG. 7. All mRNA constructs (list of constructs) elicited high, comparable and homogenous anti-CAMP2 IgG titers.

Analysis of C. acnes CAMP2 Polypeptide Glycosylation in HEK Cells

Native C. acnes CAMP2 polypeptide (SEQ ID NO: 203) contains one N-glycosylation site. Its sequence also has 15 serine and 28 threonine residues.

A Western blot using anti-CAMP2 mAb G05 was performed for recombinant C. acnes CAMP2 polypeptide (rCAMP2) and C. acnes CAMP2 polypeptides expressed in HEK cells transfected either with mRNA coding for secreted unmutated CAMP2 (CAMP2 Native) or secreted CAMP2 with mutated glycosylation site (CAMP2 Nglyc-neg). C. acnes CAMP2 polypeptides expressed by HEK cells displayed higher apparent molecular weight relative to rCAMP2 on SDS PAGE, indicating that the C. acnes CAMP2 polypeptides undergo glycosylation in HEK cells (data not shown). As expected, the molecular weight of CAMP2 Nglyc-neg was lower than unmutated CAMP2 indicating that unmutated CAMP2 underwent both N- and O-glycosylation. The protein was further shown to be highly and heterogeneously O-glycosylated as demonstrated by mass spectrometry.

Bio-layer interferometry (BLI) analyses showed that glycosylated CAMP2 was recognized by functional mAbs G05 and D07 as well as unglycosylated protein with similar binding kinetics parameters (Table 9).

TABLE 9
Affinity data mAbs and C. acnes CAMP2
polypeptides as obtained by BLI analysis

KD (M)

| | Unglycosylated | Glycosylated |
|---|---|---|
| | rCAMP2 #1 | rCAMP2-His #1 |
| mAb | (E. coli) | (HEK) |
| mAb G05 | 1.5E−9 | 1.6E−9 |
| mAb D07 | 6.2E−9 | 4.1E−9 |

### Example 4—Evaluation of Specific DsA1, DsA2 and PITP mRNA Antigens

### Example 4A: Evaluation of Specific DsA1, DsA2 and Chimeric DsA1/DsA2 mRNA Antigens

Constructs encoding C. acnes DsA1 polypeptide, C. acnes DsA2 polypeptide and chimeric C. acnes DsA1/DsA2 polypeptide were tested for their ability to elicit functional antibodies in mice:
a mRNA construct coding for the secreted form of DSA1 (“HA SS_DSA1_WT_trunc”) (SEQ ID NO: 103)
a mRNA construct coding for the secreted form of DSA2 (“HA SS_DSA2_WT_trunc”) (SEQ ID NO: 107)
a mRNA construct coding for the secreted form of DSA2 mutH (i.e. with a mutation aiming to avoid putative human cross-reactivity) (“HA SS_DSA2 mutH”) (SEQ ID NO: 108)
a mRNA construct coding for the secreted form of H4-V3 (SEQ ID NO: 113)
a mRNA construct coding for the membrane-anchored form of DSA1 (“HA SS_DSA1_HA TMB”) (SEQ ID NO: 102)
a mRNA construct coding for the membrane-anchored form of DSA1 (with a Ser-Gly-Ser linker between the antigen and the HA-TMB) (“HA SS_DSA1_HA TMB-sgs linker”) (SEQ ID NO: 104)
a mRNA construct coding for the membrane-anchored form of DSA2 (“HA SS_DSA2 HA TMB”) (SEQ ID NO: 105)
a mRNA construct coding for the membrane-anchored form of DsA2 (with a Ser-Gly-Ser linker between the antigen and the HA-TMB) (“HA SS_DSA2_HA TMB-sgs linker”) (SEQ ID NO: 106)
a mRNA construct coding for the membrane-anchored form of DSA2 mutH (“HA SS_DSA2-mutH_HA TMB”) (SEQ ID NO: 109)
a mRNA construct coding for the membrane-anchored form of DsA2 mutH (with a Ser-Gly-Ser linker between the antigen and the HA-TMB) (SEQ ID NO: 110)
a mRNA construct coding for the membrane-anchored form of DsA2 with N-glycosylation mutations (“DSA2 Nglyc-neg sgs linker”) (SEQ ID NO: 111)
a mRNA construct coding for the membrane-anchored form of DsA2 with N-glycosylation mutations (“DSA2 Nglyc-neg”) (SEQ ID NO: 112)
A formulation containing 10 μg of H4-V3 recombinant protein formulated in AlOOH (SEQ ID NO: 29)

mRNA synthesis was performed using modified nucleotides (in which all uridine nucleosides were replaced with 1-methyl pseudo uridine). The mRNA constructs were encapsulated in an LNP comprising GL-HEPES-E3-E12-DS-4-E10 as cationic lipid. LNP alone served as a negative control. Mice were immunized twice at 4 weeks interval. The mRNA content in tested formulation was of 1 μg and 5 μg per dose. The sera were obtained at day 42 following the first immunization.

DsA1, DsA2 and Chimeric DsA1/DsA2 mRNA Antigens Elicit an Anti-DsA1 IgG Response

Constructs encoding C. acnes DsA1 polypeptide, C. acnes DsA2 polypeptide and chimeric C. acnes DsA1/DsA2 polypeptide were tested for their ability to elicit an anti-DsA1 IgG response.

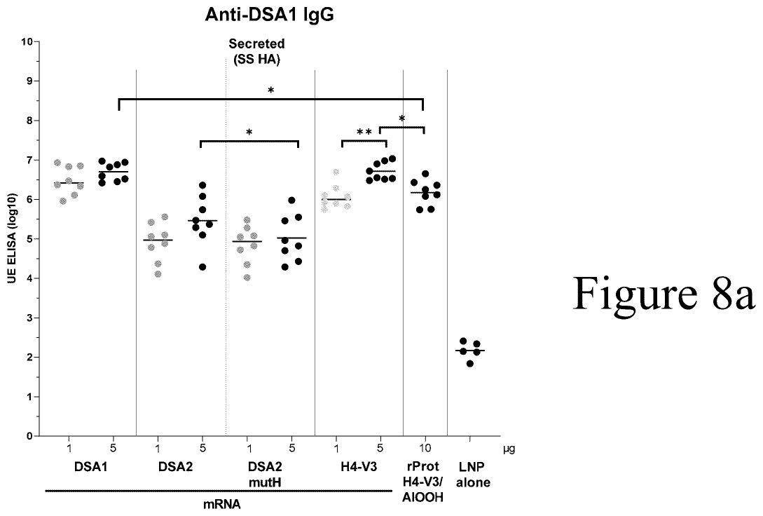

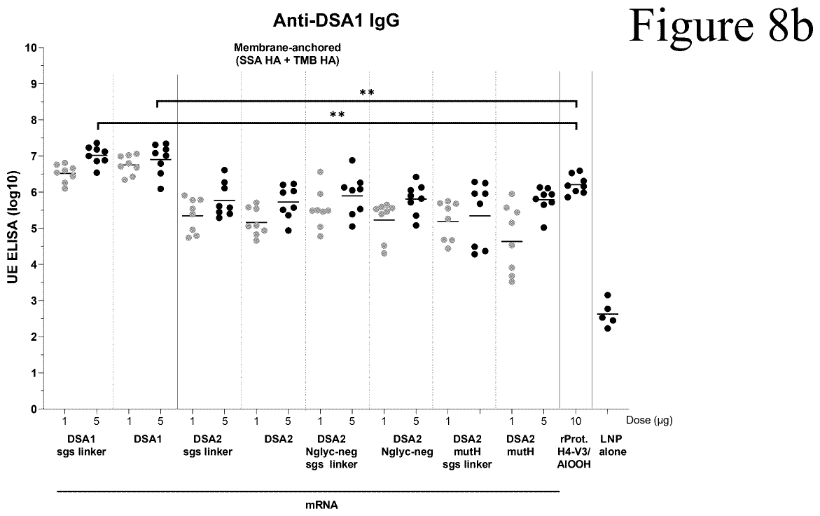

As shown in FIGS. 8A-8B, all tested formulations containing 5 μg of mRNA induced either comparable or superior ELISA IgG anti DSA1 titers relative to formulations containing lug of mRNA. All mRNA formulations containing the same mRNA quantity induced comparable ELISA IgG anti DSA1 titers which were either superior (5 μg of mRNA per dose) or equivalent (lug of mRNA per dose) relative to recombinant protein formulations.

DsA1, DsA2 and Chimeric DsA1/DsA2 mRNA Antigens Elicit an Anti-DsA2 IgG Response

Constructs encoding C. acnes DsA1 polypeptide, C. acnes DsA2 polypeptide and chimeric C. acnes DsA1/DsA2 polypeptide were tested for their ability to elicit an anti-DsA2 IgG response.

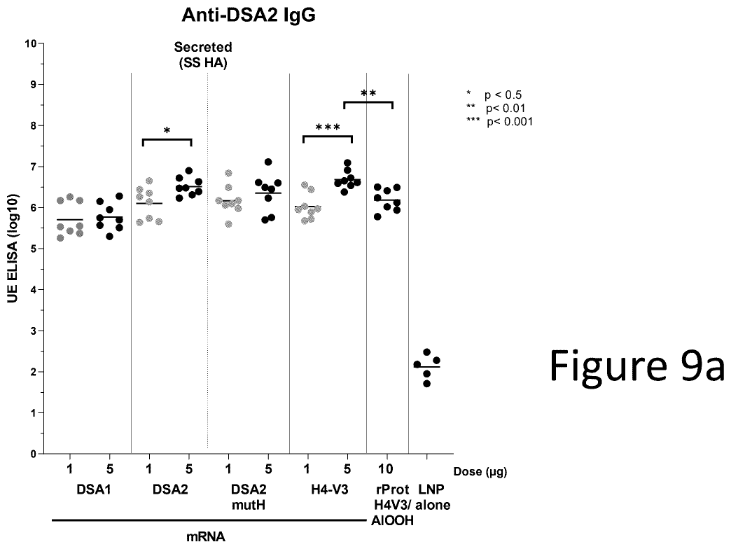

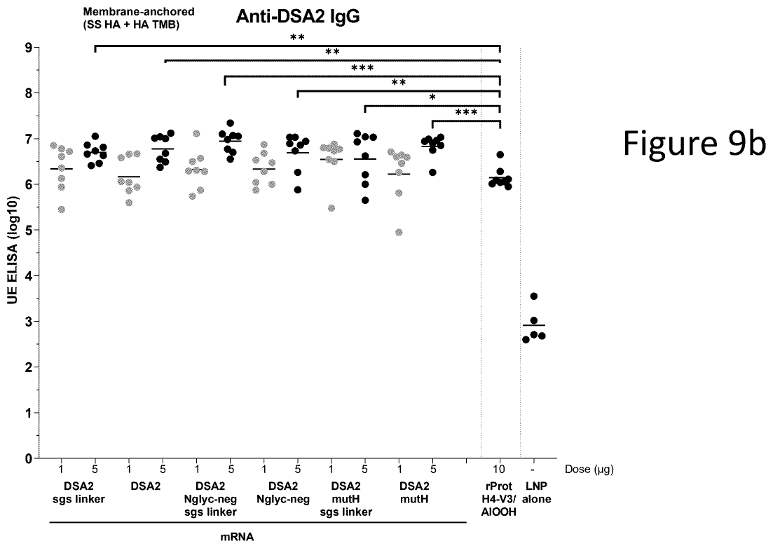

As shown in FIGS. 9A-9B, all tested formulations containing 5 μg of mRNA induced either comparable or superior ELISA IgG anti DSA2 titers than formulations containing lug of mRNA. All mRNA formulations containing the same mRNA quantity induced comparable ELISA IgG anti DSA2 titers which were either superior (5 μg of mRNA per dose) or equivalent (lug of mRNA per dose) to recombinant protein formulations.

DsA1, DsA2 and Chimeric DsA1/DsA2 mRNA Antigens Elicit Antibodies which Bind to the Surface of C. acnes

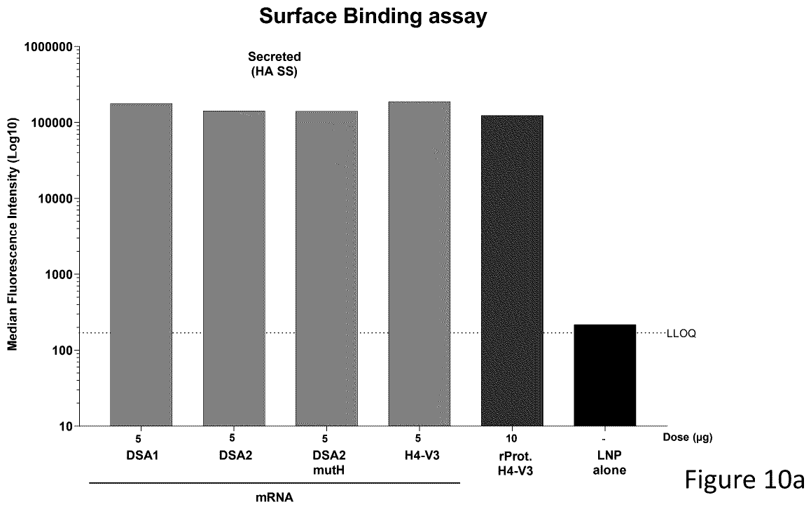

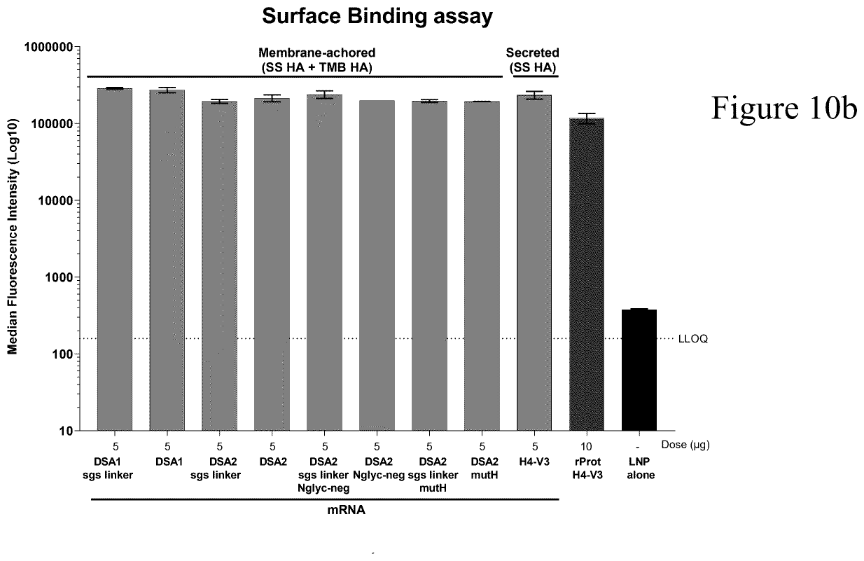

Surface Binding (SB) assays were performed on NCTC737 C. acnes strain from pools of sera generated at 5 μg dose. Results with the membrane-anchored constructs illustrate the means of 2 independent experiments. As shown in FIGS. 10A-10B, all mRNA formulations induced comparable SB titers.

DsA1, DsA2 and Chimeric DsA1/DsA2 mRNA Antigens Elicit Antibodies with Opsonophagocytic Activity

Constructs encoding C. acnes DsA1 polypeptide, C. acnes DsA2 polypeptide and chimeric C. acnes DsA1/DsA2 polypeptide were tested for their ability to elicit antibodies with opsonophagocytic activity.

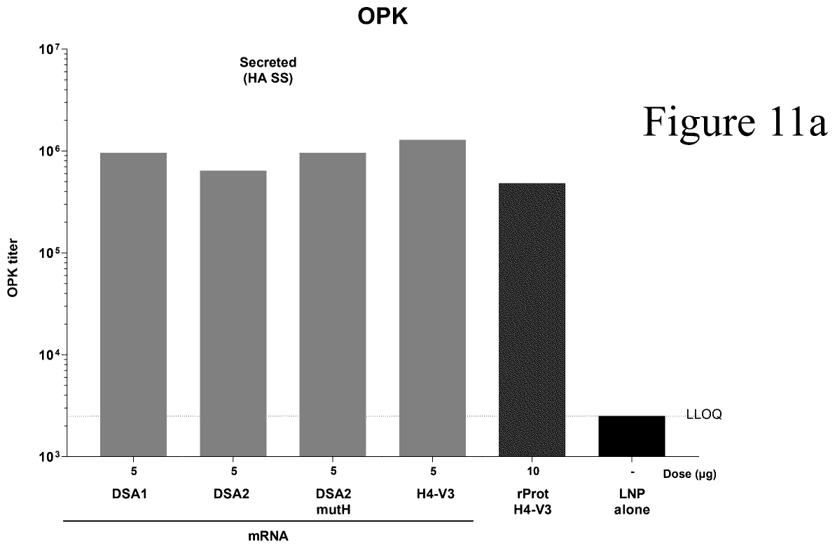

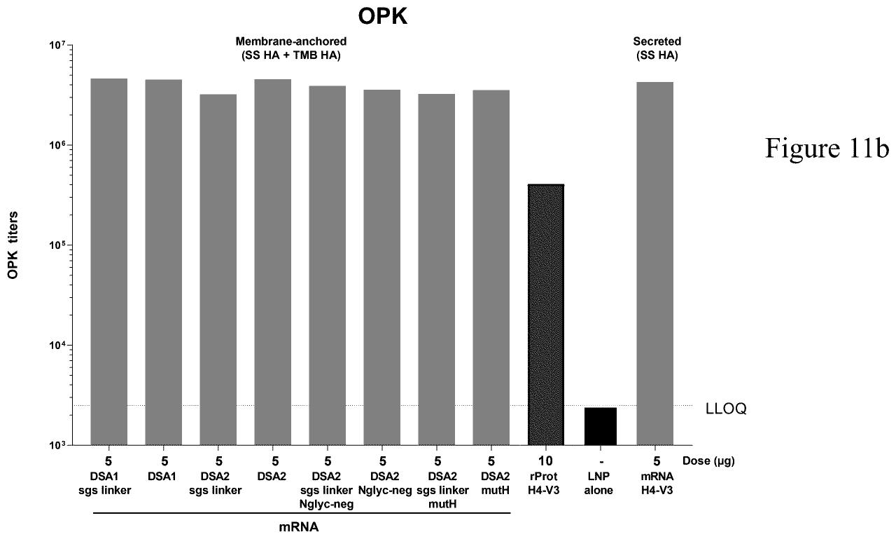

OPK assays were performed on NCTC737 C. acnes strains from pools of sera generated at 5 μg dose. Results illustrate the means of 2 (secreted constructs) or 3 (membrane-anchored constructs) independent experiments. As shown in FIGS. 11A-11B, all mRNA formulations induced comparable OPK titers.

mRNA Encoding Chimeric DsA1/DsA2 Polypeptide does not Undergo N-Glycosylation

A Western blot was performed for recombinant protein H4-V3 (SEQ ID NO: 29) and protein expressed by HEK cells transfected either with mRNA coding for secreted unmutated H4-V3 (SEQ ID NO: 113) or H4-V3 with mutated glycosylation site (H4-V3 Nglyc-neg) (SEQ ID NO: 114). Proteins expressed by HEK displayed higher molecular weight than recombinant protein H4-V3 indicating that the proteins undergo glycosylation in HEK cells (data not shown). The molecular weight of protein from H4-V3 Nglyc-neg construct appeared the same as unmutated H4-V3 construct suggesting that the protein did not undergo N-glycosylation. H4-V3 underwent only O-glycosylation.

### Example 4B: Evaluation of Specific PITP mRNA Antigens

The following mRNA constructs encoding C. acnes PITP polypeptide were tested for their ability to elicit functional antibodies in mice:
mRNA encoding secreted, native PITP (“ssHA_PITP_WT_trunc”) (SEQ ID NO: 117)
mRNA encoding secreted PITP with mutations at N-Glycosylation sites (PITP Nglyc-neg) (SEQ ID NO: 118)
mRNA encoding secreted P028-V7 (SEQ ID NO: 115)
mRNA encoding secreted P028-V7 with mutations at N-Glycosylation sites (P028-V7 Nglyc-neg) (SEQ ID NO: 119)
mRNA encoding membrane-anchored native PITP (SEQ ID NO: 120)
mRNA encoding membrane-anchored PITP with mutations at N-Glycosylation sites (PITP Nglyc-neg) (SEQ ID NO: 121)
P028-V7 recombinant protein (SEQ ID NO: 32)

mRNA synthesis was performed using modified nucleotides (in which all uridine nucleosides were replaced with 1-methyl pseudo uridine). The mRNA constructs were encapsulated in an LNP comprising GL-HEPES-E3-E12-DS-4-E10 as cationic lipid. LNP alone was used as negative control. Mice were immunized twice at 4 weeks interval. The mRNA content in tested formulation was of 5 μg per dose. A formulation containing 10 μg of P028-V7 recombinant protein formulated in AlOOH was used as a benchmark. The sera were obtained at day 42 following the first immunization.

PITP mRNA Antigens Elicit an Anti-PITP IgG Response

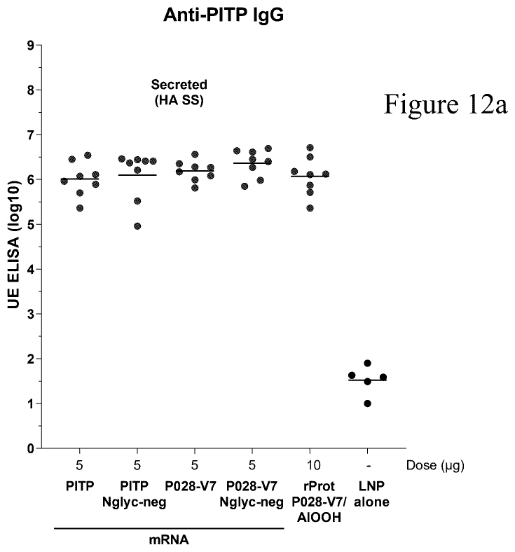

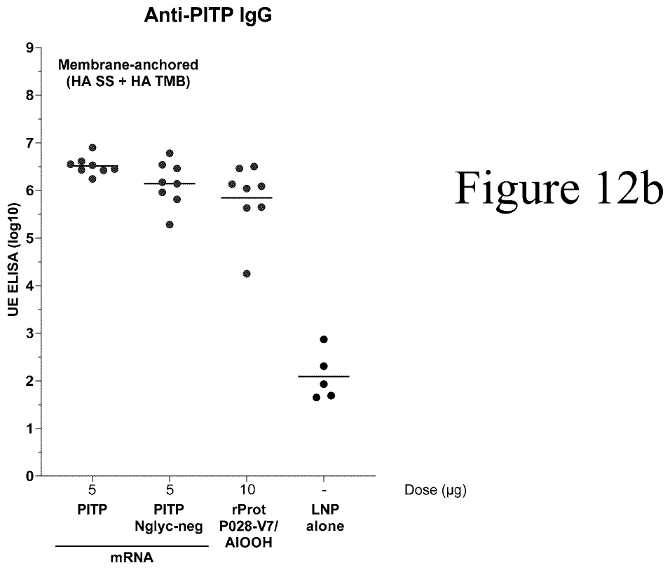

IgG anti PITP titers were measured from individual sera of mice. As shown in FIGS. 12A-12B, the membrane anchored PITP elicited superior anti-PITP titers relative to those elicited by the protein formulation. All other mRNA formulations induced IgG anti-PITP titers which were comparable to the protein formulation.

PITP mRNA Antigens Elicit Antibodies which Bind to the Surface of C. acnes

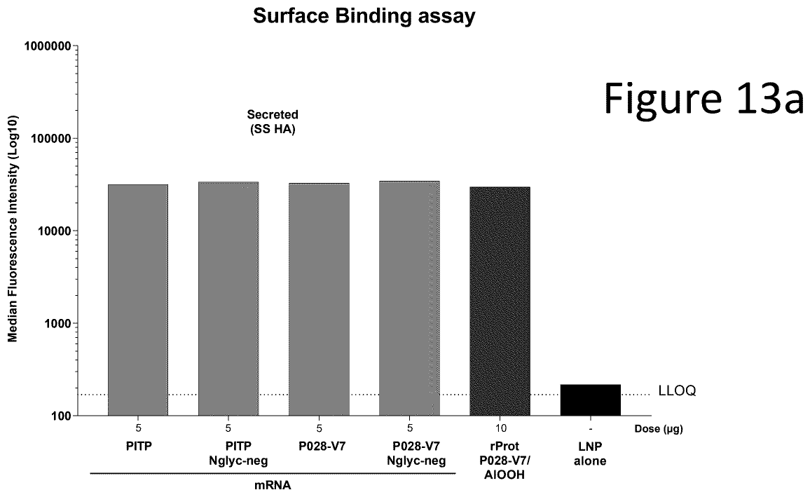

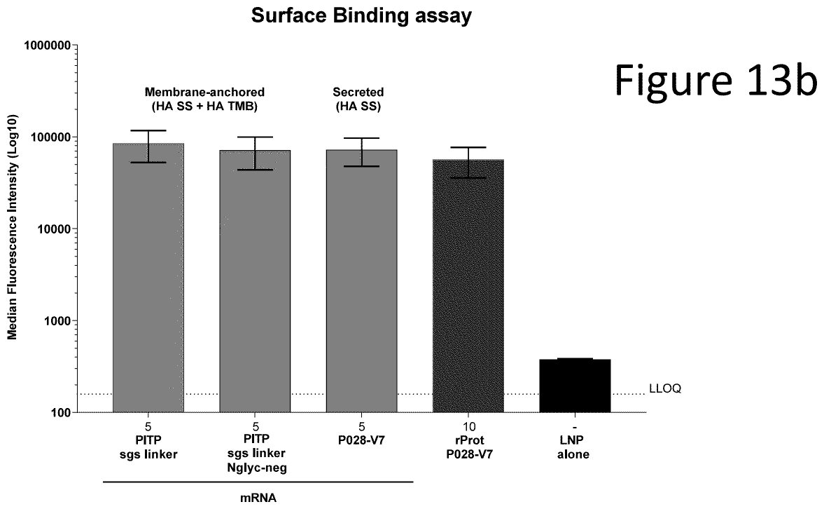

Surface Binding (SB) titers were measured on pools of sera and performed on NCTC737 C. acnes strain. As shown in FIGS. 13A-13B, all mRNA formulations induced comparable SB titers which are also comparable to the protein formulation.

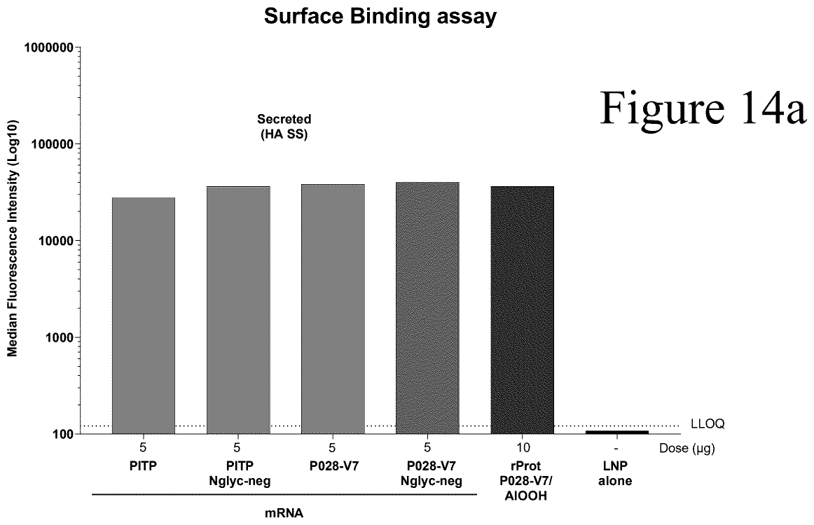

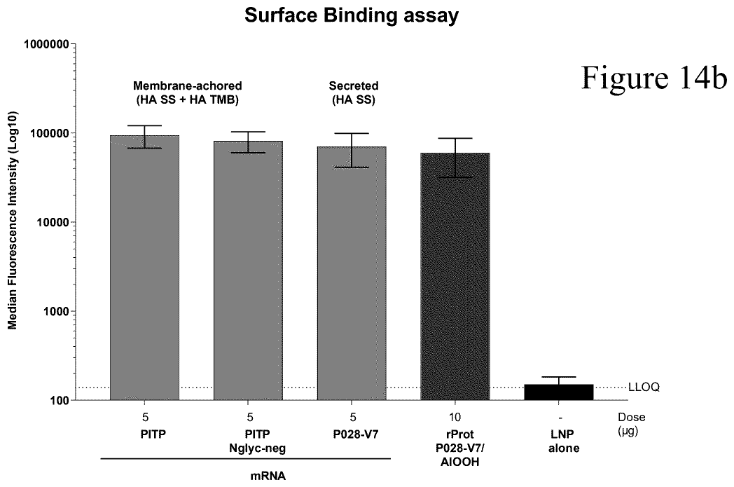

The same experiment was performed on KPA171202 C. acnes strain. As shown in FIGS. 14A-14B, all mRNA formulations induced comparable SB titers which are also comparable to the protein formulation.

PITP mRNA Antigens Elicit Antibodies with Opsonophagocytic Activity

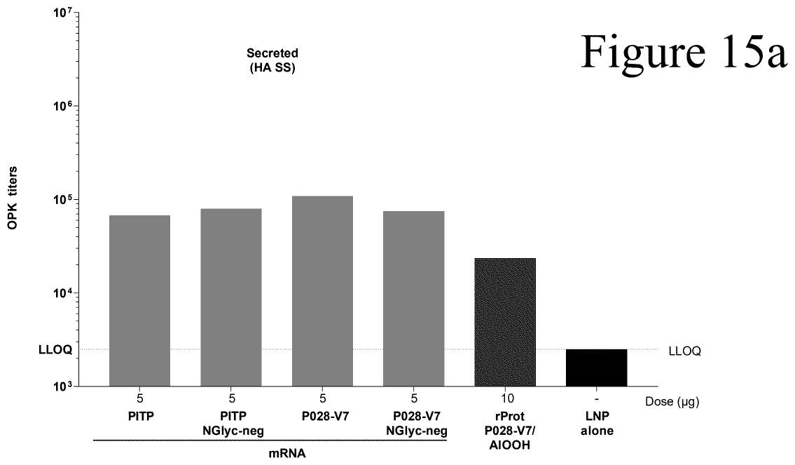

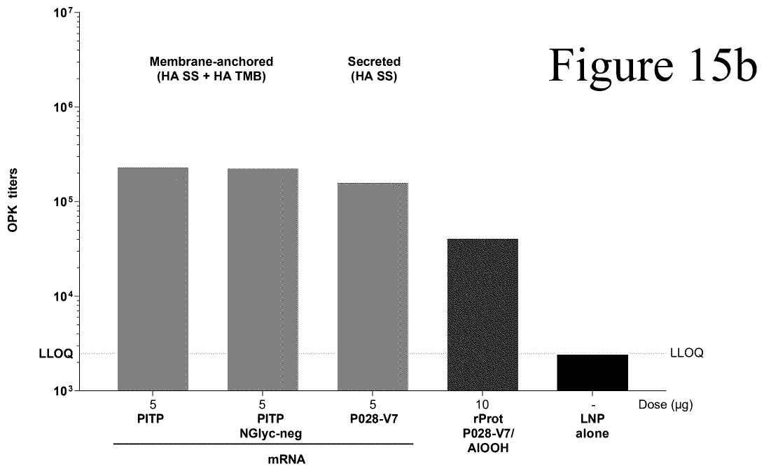

Opsonophagocytic Killing (OPK) titers were measured on NCTC737 C. acnes strains, from pools of sera of mice. Results with the membrane-anchored constructs illustrate the means of 3 independent experiments. As shown in FIGS. 15A-15B, all mRNA formulations induced comparable OPK titers which seemed measurably superior to the protein formulation.

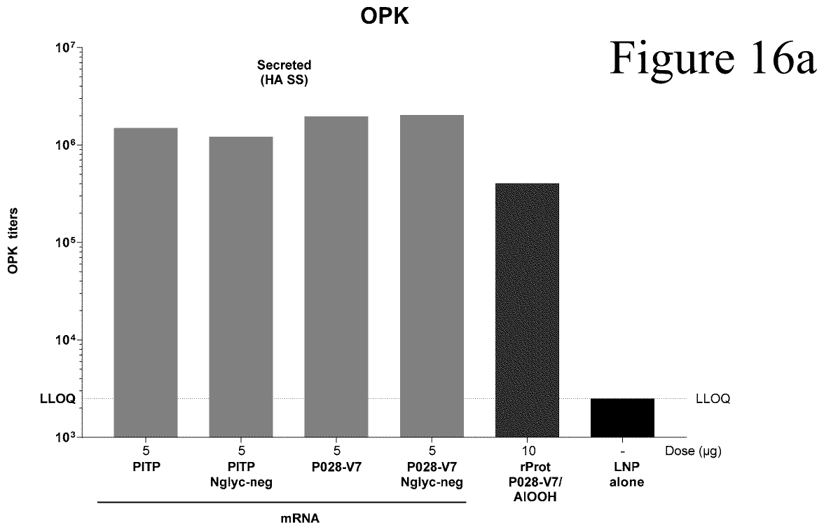

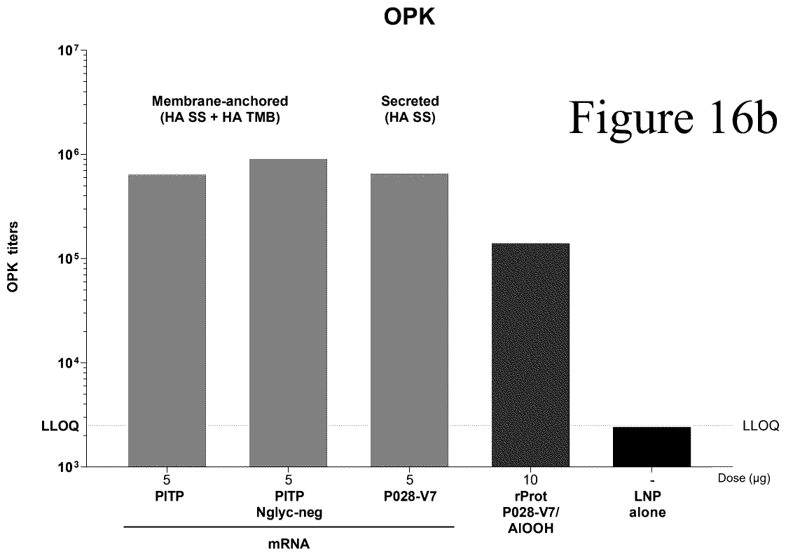

The same experiment was performed with KPA171202 C. acnes strain, except that results with the membrane-anchored constructs illustrate the means of 2 independent experiments. As shown in FIGS. 16A-16B, all mRNA formulations induced comparable OPK titers which seemed measurably superior to the protein formulation.

### Example 5-Immunisation with mRNA H4-V3 and mRNA P28-V7 Gives Rise to Antibody Responses which Recognise a Range of C. acnes Strains (Cross-Reactivity)

mRNA synthesis was performed using modified nucleotides (in which all uridine nucleosides were replaced with 1-methyl pseudo uridine). The mRNA constructs were encapsulated in an LNP comprising GL-HEPES-E3-E12-DS-4-E10 as cationic lipid. LNP alone served as a negative control. Mice were immunized twice at 4 weeks interval with the same mRNA antigens as described in Example 4 as illustrated in FIGS. 10a, 11a (DsA1 & DsA2 constructs respectively) and 16a (PITP constructs). The mRNA content in tested formulation was of 1 μg and 5 μg per dose. The sera were obtained at day 42 following the first immunization.

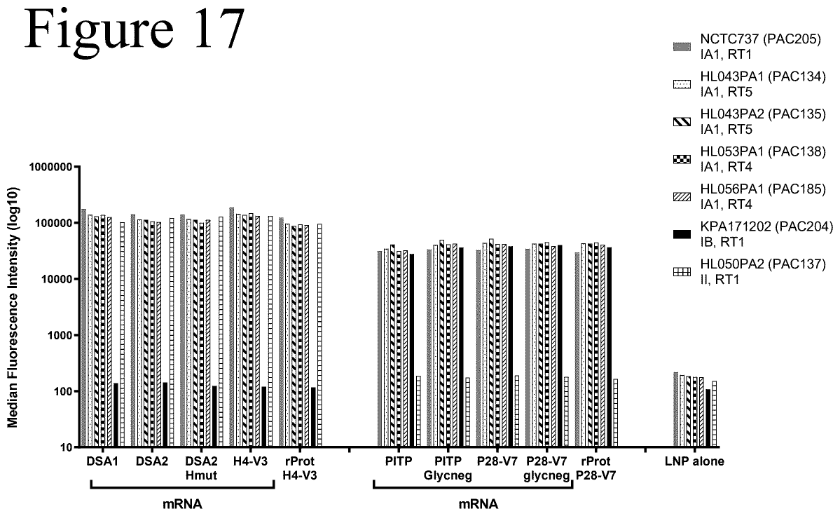

Surface Binding (SB) titers performed on C. acnes strains NCTC737, KPA171202 and five other strains (as listed in Table 10), were evaluated from pools of sera. The strains were selected to reflect the phylotype proportion found in acne vulgaris and based on i) the level of antigen expression and ii) ribotype. The results for the 5 μg dosage are shown in FIG. 17. mRNA encoding secreted H4-V3 and mRNA encoding secreted P028-V7 performed at least as well as other mRNA constructs encoding secreted DsA1, DsA2 or PITP antigens or their recombinant protein counterparts, against a range of C. acnes strains.

TABLE 10
Strain collection used in Example 5

| | Alternative | | Ribotype | | Reference for phylogenic |
|---|---|---|---|---|---|
| Strain | strain name | Phylotype | (RT) | Source | analysis |
| NCTC737 | PAC205 | IA1 | 1 | NCTC | Liu et al. 2014 |
| HL043PA1 | PAC134 | IA1 | 5 | BEI | McDowell et al. 2012 |
| | | | | Ressources | Tomida et al. 2013 |
| HL043PA2 | PAC135 | IA1 | 5 | BEI | McDowell et al. 2012 |
| | | | | Ressources | Tomida et al. 2013 |
| HL053PA1 | PAC138 | IA1 | 4 | BEI | McDowell et al. 2012 |
| | | | | Ressources | Tomida et al. 2013 |
| HL056PA1 | PAC185 | IA1 | 4 | BEI | McDowell et al. 2012 |
| | | | | Ressources | Tomida et al. 2013 |
| KPA171202 | PAC204 | IB | 1 | DSMZ | Bruggeman et al. 2004 |
| | | | | | Brzuszkiewicz et al. 2011 |
| | | | | | Tomida et al. 2013 |
| HL050PA2 | PAC137 | II | 1 | BEI | Tomida et al. 2013 |
| | | | | Ressources | |

### REFERENCES

Bruggemann, H., Henne, A., Hoster, F., Liesegang, H., Wiezer, A., Strittmatter, A., Hujer, S., Durre, P., and Gottschalk, G. “The complete genome sequence of Propionibacterium acnes, a commensal of human skin.” Science (2004) 305:671-673.
Brzuszkiewicz, E.; Weiner, J.; Wollherr, A.; Thürmer, A.; Hüpeden, J.; Lomholt, H. B.; Kilian, M.; Gottschalk, G.; Daniel, R.; Mollenkopf, H. J.; et al. Comparative genomics and transcriptomics of Propionibacterium acnes. PLOS One (2011), 6: e21581
Liu J, Cheng A, Bangayan N J, Barnard E, Curd E, Craft N, Li H. Draft Genome Sequences of Propionibacterium acnes Type Strain ATCC6919 and Antibiotic-Resistant Strain HL411PA1. Genome Announc. (2014) 2: e00740-14.
McDowell A, Barnard E, Nagy I, Gao A, Tomida S, Li H, Eady A, Cove J, Nord C E, Patrick S. An expanded multilocus sequence typing scheme for Propionibacterium acnes: investigation of ‘pathogenic’, ‘commensal’ and antibiotic resistant strains. PLOS One. (2012) 7: e41480
Tomida S, Nguyen L, Chiu B H, Liu J, Sodergren E, Weinstock G M, Li H. Pan-genome and comparative genome analyses of Propionibacterium acnes reveal its genomic diversity in the healthy and diseased human skin microbiome. mBio. (2013) 4: e00003-13.

### Example 6—CAMP2 mRNA Construct Synthesis and Reproducibility

Synthesis and Characterisation of mRNA Encoding C. acnes CAMP 2 Polypeptide

mRNA size, tail size, % of tailed population and % cap1 were evaluated for 4 different batches of mRNA encoding C. acnes CAMP2 (polypeptide sequence according to SEQ ID NO: 5) produced from 2 different optimized plasmid sequences (mRNA #1 CAMP2 (SEQ ID NO: 90) and mRNA #2 CAMP2 (SEQ ID NO: 91), respectively) (2 batches/plasmid sequence: batch 1 and batch 2). mRNA synthesis was performed using modified nucleotides (in which all uridine nucleosides were replaced with 1-methyl pseudo uridine). With respect to mRNA size, tail size, % of tailed population and cap1%, all mRNAs met the targeted specifications. Sequencing analyses confirmed that the expected mRNA sequences were generated.

Characterisation of mRNA-LNP Formulation

LNP size, % encapsulation (% EE) and mRNA integrity were measured for the 4 batches of mRNA produced from mRNA #1 CAMP2 (SEQ ID NO: 90) and mRNA #2 CAMP2 (SEQ ID NO: 91) (corresponding to two different codon optimizations). Each mRNA antigen was encapsulated in an LNP comprising GL-HEPES-E3-E12-DS-4-E10 as cationic lipid. With respect to LNP size and % of encapsulation (% EE), all LNPs fell into the expected size range and the targeted % EE.

### Mouse Immunisation

The 4 batches of CAMP2 mRNA were tested for their ability to elicit functional antibodies in mice:

Mice were immunized twice at 4 weeks interval, with either mRNA #1 CAMP2 (batch 1), mRNA #1 CAMP2 (batch 2), mRNA #2 CAMP2 (batch 1) or mRNA #2 CAMP2 (batch 2) encapsulated in LNP. The mRNA content in tested formulation was of 1 μg per dose. A formulation containing 10 μg of CAMP2 recombinant protein formulated in AlOOH was implemented as a benchmark. The sera were obtained at day 42 following the first immunization. LNP alone was used as negative control.

The weight gain over time of mice immunized with mRNA encoding C. acnes CAMP2 polypeptide was assessed. No swelling was observed at the site of injection and regular weight gain was monitored over the study period for all experimental groups (hence no local reaction and no impact on body weight).

Immunisation with C. acnes CAMP2 Antigens Elicits an Anti-CAMP2 IgG Response

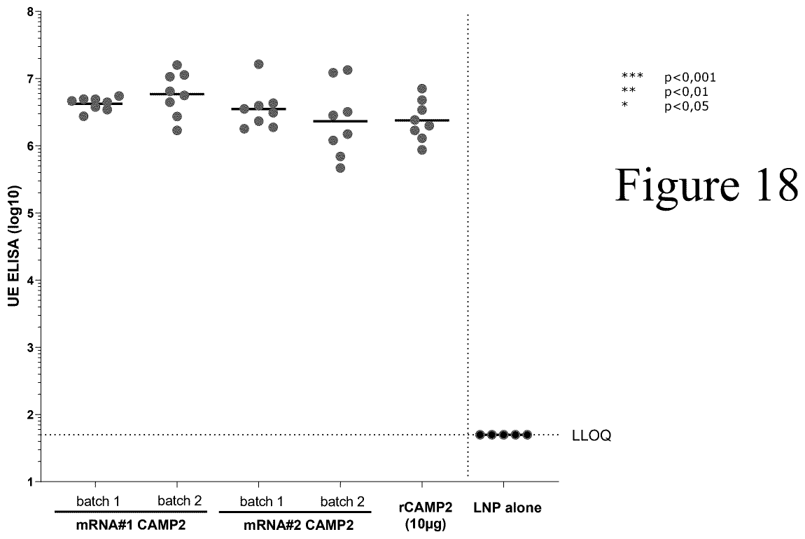

IgG anti CAMP2 titers were measured from individual sera of mice at day 42 following the first immunization and evaluated by ELISA. As shown in FIG. 18, all mRNA formulations induced comparable IgG anti CAMP2 titers, which were also comparable to those elicited by the protein formulation.

mRNA Encoding C. acnes CAMP2 Polypeptides Decreases the Co-Hemolytic Activity of CAMP 2

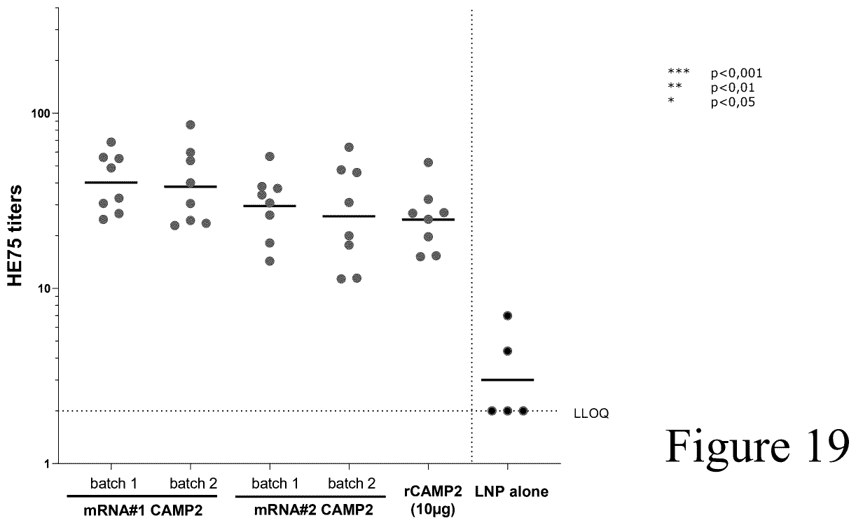

Co-hemolytic neutralizing titers were measured for IgG purified from individual sera of mice. As shown in FIG. 19, all mRNA formulations induced comparable neutralizing titers which were also comparable to the protein formulation. Correlation between ELISA IgG anti CAMP2 titers and co-hemolytic neutralizing titers was good (R=0.76).

### Example 7—Combinations of mRNA

Design of C. acnes Antigen Constructs

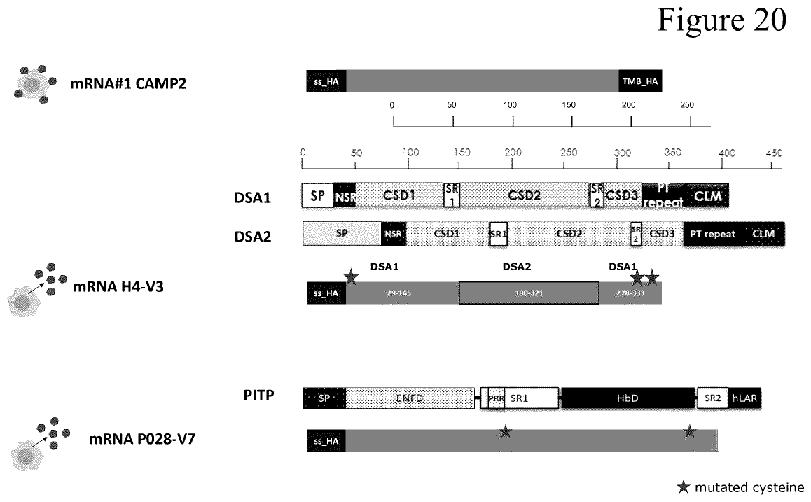

The schematic visualization of the protein domains of DSA1, DSA2 and PITP, and the mRNA constructs that have been synthesized based on these proteins: mRNA H4-V3 (a chimera of DSA1 and DSA2) and mRNA P028-V7 (derived from PITP) as well as the mRNA construct of mRNA #1 CAMP2, is provided in FIG. 20.

The schematic visualization of FIG. 20 provides details on protein domains of DSA1, DSA2 and PITP, and the 2 mRNA constructs that have been synthesized based on these proteins: mRNA H4-V3 (a chimeric C. acnes DsA1/DsA2 construct) (SEQ ID NO: 113) and mRNA P028-V7 (derived from PITP) (SEQ ID NO: 115), as well as the mRNA construct coding for CAMP2, mRNA #1 CAMP2 (SEQ ID NO: 90). The CAMP2 mRNA #1 construct comprised an unmutated CAMP2 sequence, the signal sequence (HA SS) and the transmembrane domain sequence (HA TMB) of Influenza Hemagglutinin (HA, H1N1 A/Caledonia/20/1999) at the N-terminus and C-terminus, respectively. The H4-V3 and P028-V7 mRNA constructs comprised the protein sequence and the signal sequence (HA SS) of Influenza Hemagglutinin (HA, H1N1 A/Caledonia/20/1999) at the N-terminus. CAMP2, H4-V3 and P028-V7 sequences were derived from the protein sequence of C. acnes strain KPA171202. None of the protein sequences contained the native signal sequence of the proteins.

Synthesis and Characterisation of mRNA Encoding C. acnes Polypeptides

mRNA synthesis was performed using modified nucleotides (in which all uridine nucleosides were replaced with 1-methyl pseudo uridine). mRNA size, tail size, % of tailed population and % cap1 were evaluated for the synthesized mRNA constructs described in FIG. 20: mRNA H4-V3 (SEQ ID NO: 113), mRNA P028-V7 (SEQ ID NO: 115) and mRNA #1 CAMP2 (SEQ ID NO: 90). With respect to mRNA size, tail size, % of tailed population and cap1%, all mRNAs met the targeted specifications. Sequencing analyses confirmed that the expected mRNA sequences were generated (data not shown).

In Vitro Expression of mRNA Encoding C. acnes Polypeptides in HEK Cells

Western blots were run for in vitro expression products in HEK cells of synthesized mRNA H4-V3, mRNA P028-V7 and mRNA #1 CAMP2 (constructs described in FIG. 20), 48 h after transfection. The proteins were detected in the expected compartment, namely in the membrane fraction for CAMP2 and in the supernatant for H4-V3 and P028-V7.

Characterisation of mRNA-LNP Formulation

LNP size, % encapsulation (% EE) and mRNA integrity were evaluated for mRNA H4-V3, mRNA P028-V7 and mRNA #1 CAMP2 (constructs described in FIG. 20) individually encapsulated or co-encapsulated in an LNP. The LNP comprised OF-02, cKK-E10 or GL-HEPES-E3-E12-DS-4-E10, as cationic lipid. With respect to LNP size and % of encapsulation (% EE), all LNPs fell into the expected size range and the targeted % EE. mRNA integrity in all LNPs was as expected.

### Example 7A: Evaluation of mRNA Combinations in Different Lipid Formulations

The following constructs were used throughout Example 7A: mRNA constructs coding for the membrane-anchored form of CAMP2 (SEQ ID NO: 90) and the secreted forms of H4-V3 (SEQ ID NO: 113) and P028-V7 (SEQ ID NO: 115) were tested either alone or in combination: [H4-V3 plus P028-V7] or [H4-V3 plus P028-V7 plus CAMP2]. A co-mix formulation with GL-HEPES-E3-E12-DS-4-E10 LNP and the three mRNA constructs (i.e. wherein the three mRNA constructs were encapsulated separately in the LNPs) was also tested. GL-HEPES-E3-E12-DS-4-E10 LNP alone was used as negative control.

Immunisation of Mice with Formulations Containing mRNA Combinations, and Monitoring of Mice

Mice were monitored for changes in body weight and temperature after immunization twice at 4 weeks interval, with either of the following mRNA constructs, encapsulated (one mRNA construct) or co-encapsulated (two or three mRNA constructs) in LNPs comprising OF-02, cKK-E10 or GL-HEPES-E3-E12-DS-4-E10 as cationic lipid. mRNA constructs coding for the membrane-anchored form of CAMP2 and the secreted forms of H4-V3 and P028-V7 were tested either alone or in combination: [H4-V3 plus P028-V7] or [H4-V3 plus P028-V7 plus CAMP2]. A co-mix formulation with GL-HEPES-E3-E12-DS-4-E10 LNP and the three mRNA constructs (i.e, wherein the three mRNA constructs were encapsulated separately in the LNPs) was also tested. GL-HEPES-E3-E12-DS-4-E10 LNP alone was used as negative control. mRNA synthesis was performed using modified nucleotides (in which all uridine nucleosides were replaced with 1-methyl pseudo uridine). The mRNA content for each construct in tested formulations was of 1 μg per dose. All tested formulations showed no impact on weight gain nor changes in body temperatures.

Mice were also monitored for swelling at the injection site. We documented higher frequencies of mice with swelling after boosting than after priming. Bi- or tri-valent formulation provoked higher swelling frequency than monovalent formulations. Overall, the OF-02 containing formulations were shown to be more reactogenic than cKK-E10 and GL-HEPES-E3-E12-DS-4-E10-containing ones, which were comparable.

Formulations Containing mRNA Encoding C. acnes CAMP 2 in Combination with mRNAs Encoding Other C. Acnes Antigens Decrease the Co-Hemolytic Activity of CAMP 2

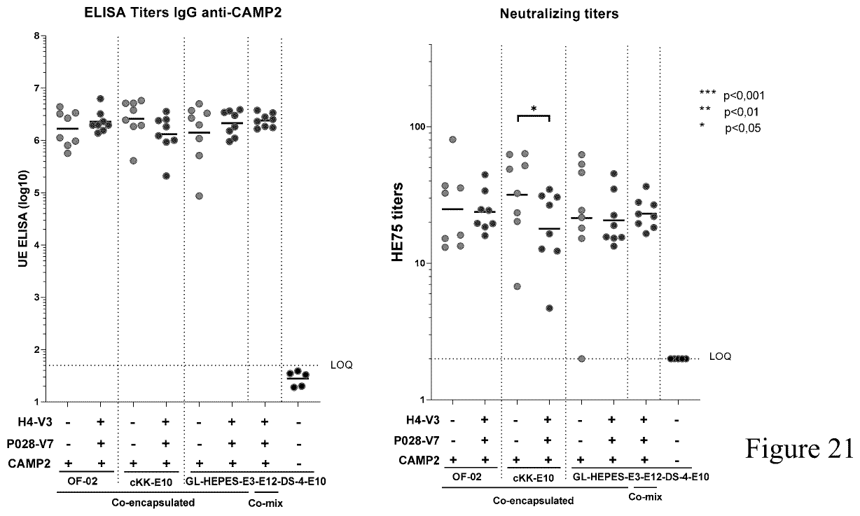

FIG. 21 shows ELISA IgG anti CAMP2 titers and co-hemolytic neutralizing titers of IgG purified from individual sera of mice immunized twice at 4 weeks interval, with the mRNA antigens as described above. As shown in FIG. 21, no interferences were monitored as CAMP2 mRNA responses were comparable between co-encapsulated mRNA and CAMP2 mRNA on its own. Co-encapsulated and co-mixed formulations also showed comparable responses for both assays.

Formulations Containing Combinations of mRNAs Encoding C. acnes Antigens Give Rise to IgG Responses

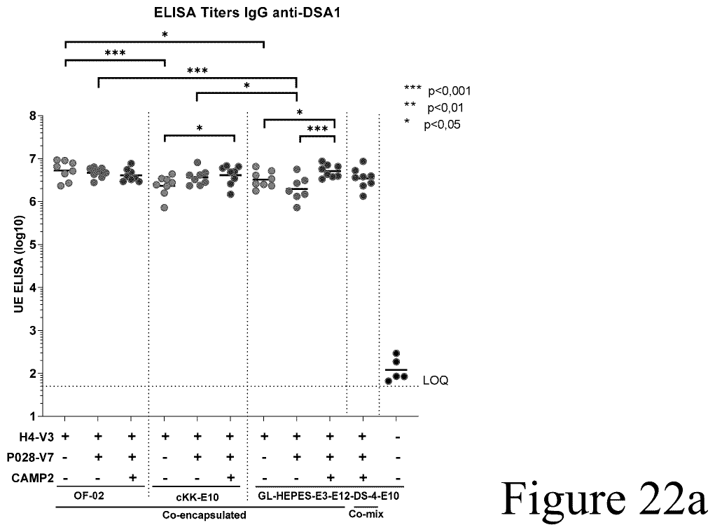

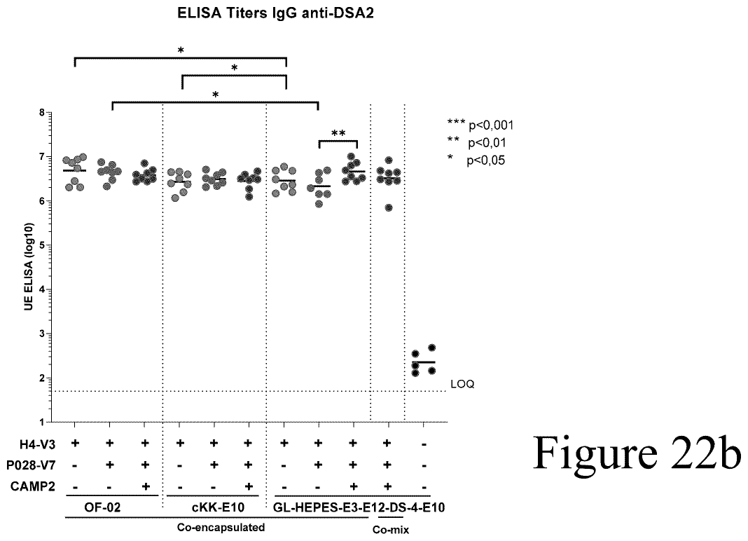

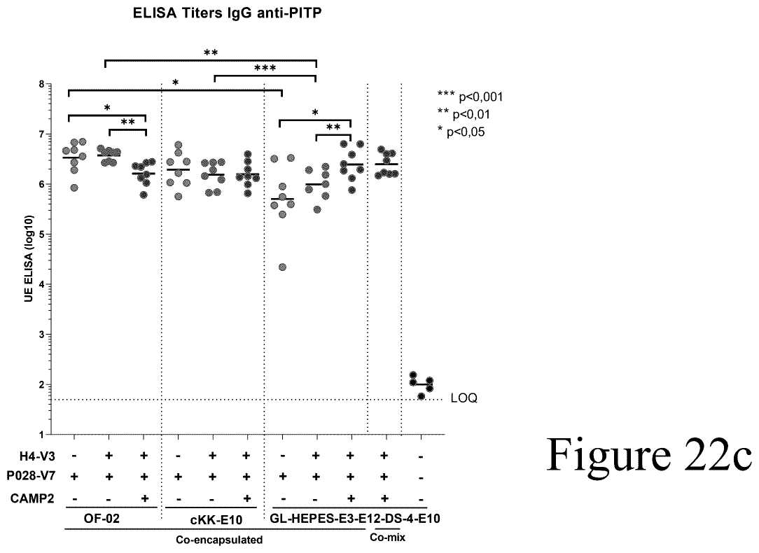

IgG anti-DSA1, -DSA2 and -PITP titers from individual sera of mice immunized twice at 4 weeks interval with the mRNA antigens as described above, were tested. As shown in FIGS. 22A-22C, ELISA IgG showed that the anti-DSA1, -DSA2 and -PITP titers were largely comparable when the three mRNAs were co-encapsulated or co-mixed (tri-valent combinations). No overall interference was detected compared to correspondent mono-valent formulation, whatever the cationic lipid in the LNP. Little interferences were observed when each mRNA was formulated either alone or in a bi-valent combination, with OF-02 LNP significantly more potent at inducing anti-DSA1, -DSA2 and -PITP titers compared to cKK-E10 & GL-HEPES-E3-E12-DS-4-E10 which were comparable.

Formulations Containing Combinations of mRNAs Encoding C. acnes Antigens Give Rise to Antibodies which Bind to the Surface of C. acnes, and with Opsonophagocytic Activity

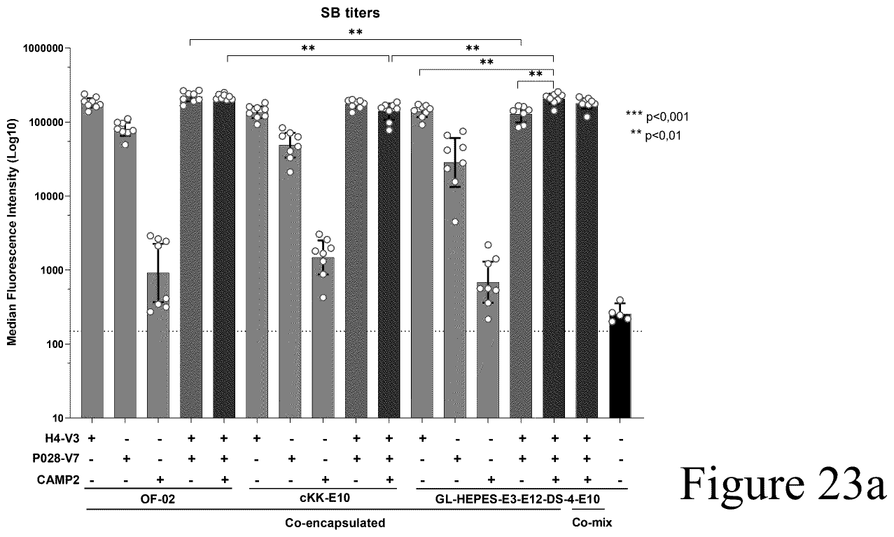

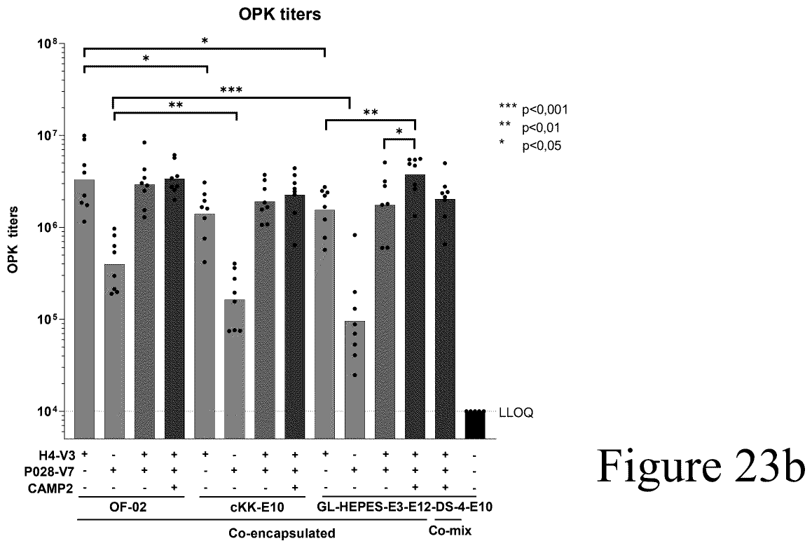

Opsonophagocytic Killing (OPK) titers and Surface Binding (SB) titers, were both performed on NCTC737 C. acnes strains, from individual sera of mice immunized twice at 4 weeks interval with the mRNA antigens as described above. As shown in FIGS. 23A-23B, both assays documented comparable or superior titers when the three mRNAs were co-encapsulated or co-mixed (tri-valent combinations). No overall interference was detected compared to correspondent mono-valent formulation, whatever the cationic lipid in the LNP. No interferences were observed when each mRNA was formulated either alone or in a bi-valent combination, however the functional responses were dominated by H4-V3 antigen. OF-02 LNP was significantly more potent at inducing OPK titers compared to cKK-E10 & GL-HEPES-E3-E12-DS-4-E10 which were comparable.

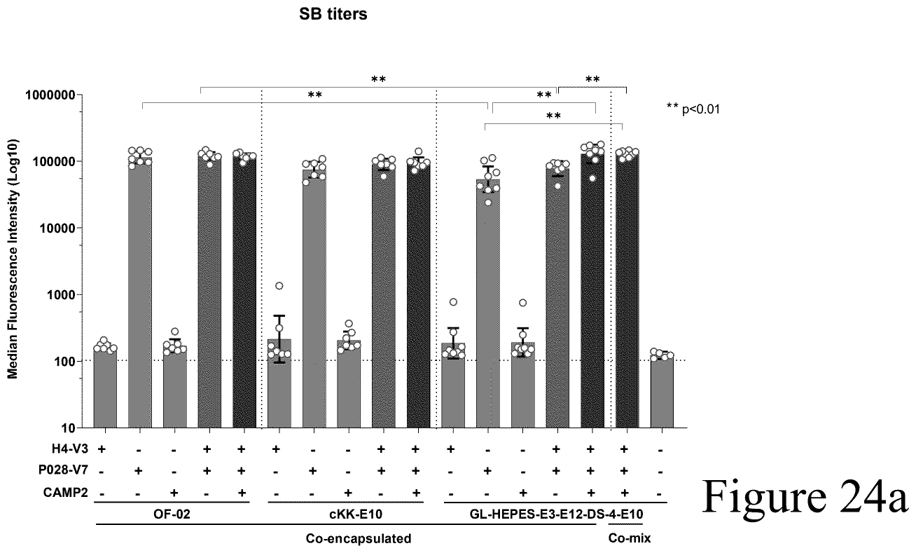

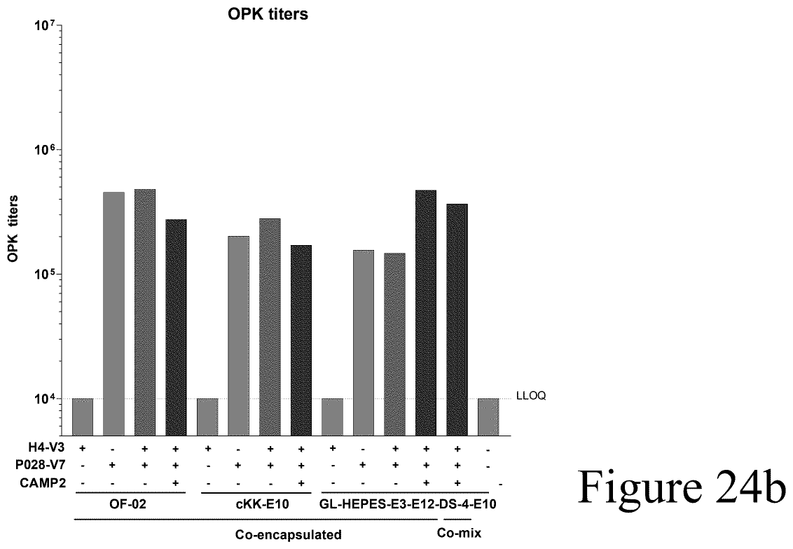

The same experiment was repeated with KPA171202 C. acnes strains, with individual sera of mice (for the SB assay) or pool of sera (for the OPK assay). As shown in FIGS. 24A-24B, both assays documented comparable or superior titers when the three mRNAs were co-encapsulated or co-mixed (tri-valent combinations). No overall interference was detected compared to correspondent mono-valent formulation, whatever the cationic lipid in the LNP. No interferences were observed when each mRNA was formulated either alone or in a bi-valent combination, however the functional responses were dominated by H4-V3 antigen. OF-02 LNP significantly more potent at inducing OPK titers compared to cKK-E10 & GL-HEPES-E3-E12-DS-4-E10 which were comparable

### Example 7B: Mode of Action of the Combination Vaccine

Correlations Between Antibody Responses Elicited by H4-V3, P28-V7 and CAMP 2 mRNAs

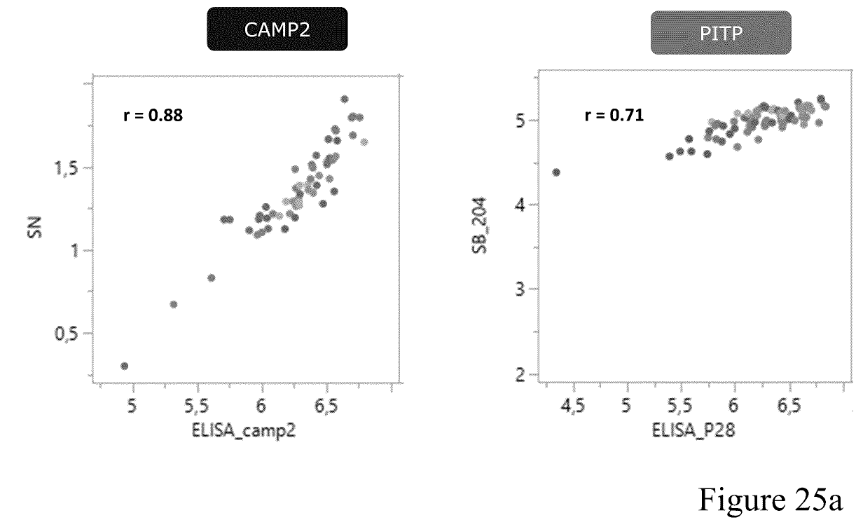

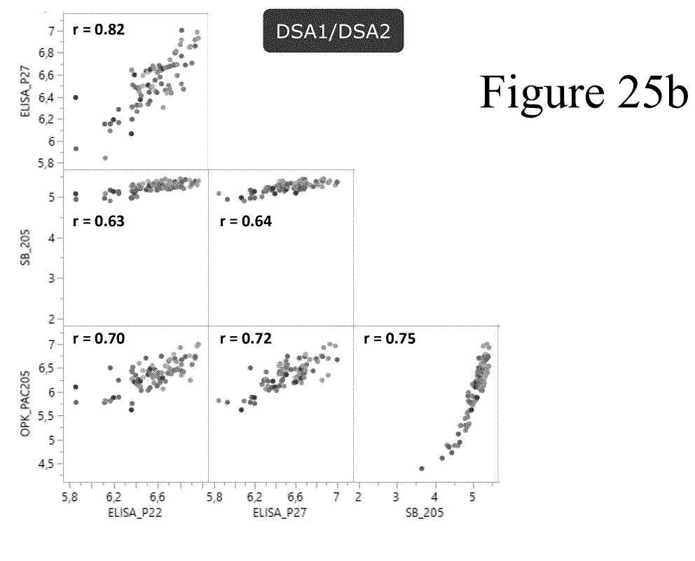

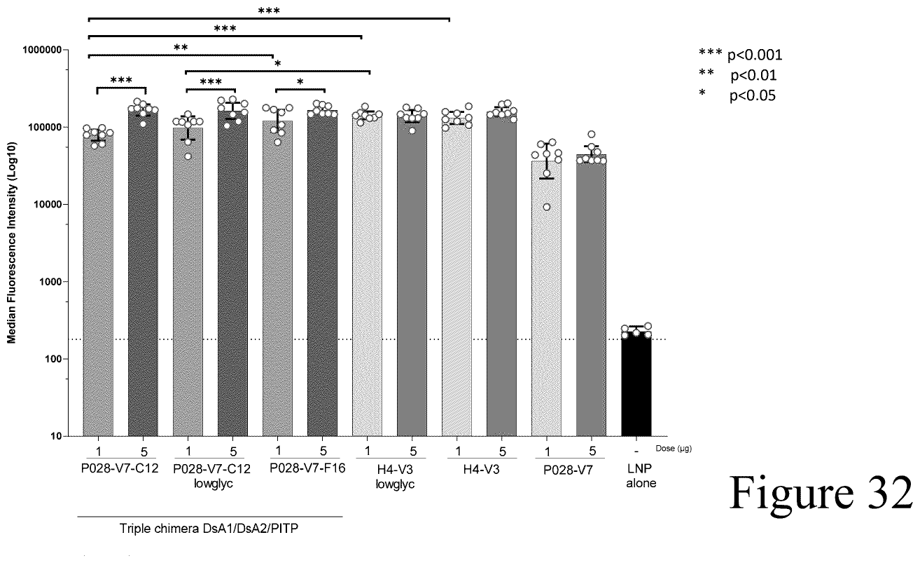

The Pearson correlations between ELISA IgG anti-DSA1, -DSA2, -PITP, and -CAMP2 titers and co-hemolytic neutralizing titers (CAMP2) or Surface Binding titers (PITP, DSA1 and DSA2), or Opsonophagocytic Killing titers (DSA1 and DSA2)) both performed on either KPA 171202 (PITP) or NCTC737 (DSA1/DSA2) C. acnes strains, from individual sera of mice immunized twice at 4 weeks interval with the same mRNA antigens as described in FIG. 32 and encapsulated in the same LNPs, were analysed. The results are shown in FIGS. 25A-25B. r=Pearson correlation factor calculated between each variable. All correlated assays exhibited good r values.

### Example 8—Protein-Based Vaccines and Choice of Adjuvants

Throughout Example 8, the following recombinant proteins were tested: H4-V3 (SEQ ID NO: 29), P028-V7 (SEQ ID NO: 32) and CAMP2 (SEQ ID NO: 1).

Formulations Containing Combinations of C. acnes Proteins Give Rise to IgG Responses

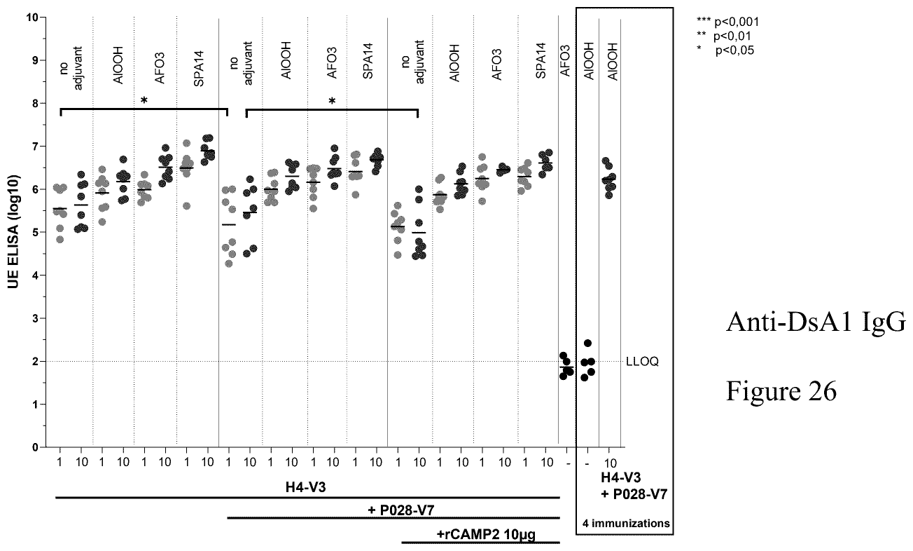

ELISA IgG anti DSA1 titers were measured on individual sera of mice immunized twice at 4 weeks interval with H4-V3 (SEQ ID NO: 29), P028-V7 (SEQ ID NO: 32) and CAMP2 (SEQ ID NO: 1) recombinant proteins, formulated either alone or in combination [H4-V3 plus P028-V7] or [H4-V3 plus P028-V7 plus CAMP2]. These formulations were done with AlOOH, AF03, SPA14 adjuvants or with no adjuvant. An additional group of mice received 4 immunizations of the bivalent vaccine [H4-V3 plus P028-V7] in AlOOH at 2 weeks interval. The sera were obtained at day 42 following the first immunization. AlOOH and AF03 alone were used as negative control. The H4-V3 and P028-V7 recombinant protein content in tested formulation was of 1 μg or 10 μg of each per dose (in mono-, bi- and tri-valent). The CAMP2 recombinant protein content in tested formulation was 10 μg per dose (in mono- and tri-valent). As shown in FIG. 26, ELISA IgG showed that the anti-DSA1 titers were largely comparable when the three recombinant proteins were co-formulated (bi- and tri-valent combinations). No overall interference was detected compared to correspondent mono-valent formulation, whatever the adjuvant. Little interferences were observed at lug in experimental group with no adjuvant. Among adjuvanted groups, SPA14- and AlOOH-containing formulations elicited the highest and the lowest ELISA IgG anti DSA1 titers, respectively. The ELISA IgG anti DSA1 titers were comparable between groups which received 2 and 4 immunisations, 4 and 2 weeks apart respectively. Anti-DSA2 IgG response pattern was similar (data not shown).

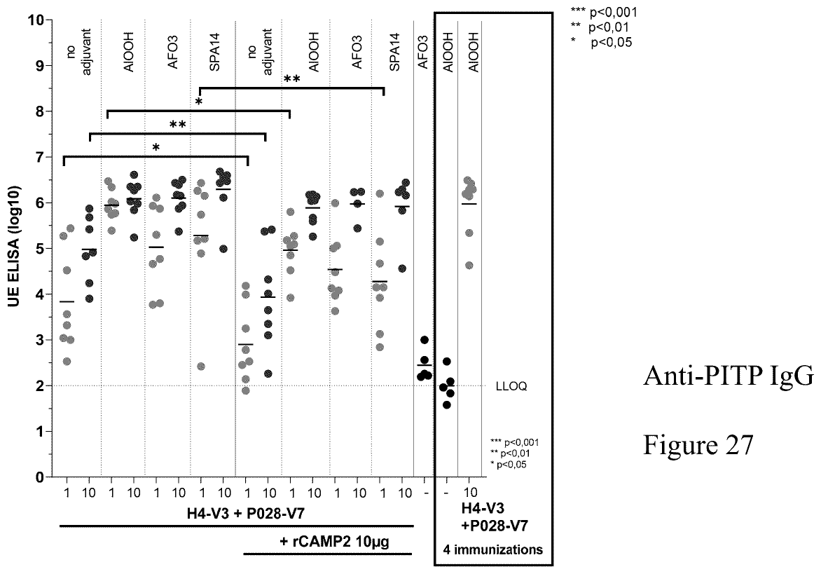

ELISA IgG anti PITP titers from individual sera of mice immunized twice at 4 weeks interval with the same recombinant protein antigens as described in FIG. 26 and formulated in the same adjuvants. As shown in FIG. 27, ELISA IgG showed that the anti-PITP titers were largely comparable when the three recombinant proteins were co-formulated at 10 μg of each recombinant protein per dose (bi-valent and tri-valent formulations). At this 10 μg per antigen dose, no overall interference was observed whatever the adjuvant which elicited comparable titers. Low and heterogenous ELISA IgG anti PITP titers were documented at lug of each recombinant protein per dose, excepted in bivalent formulation at 1 μg formulated in AlOOH. The ELISA IgG anti PITP titers were comparable between groups which received 2 and 4 immunisations, 4 and 2 weeks apart respectively.

Formulations Containing Combinations of C. acnes Proteins Including C. acnes CAMP 2 Give Rise to IgG Responses and Decrease the Co-Hemolytic Activity of CAMP 2

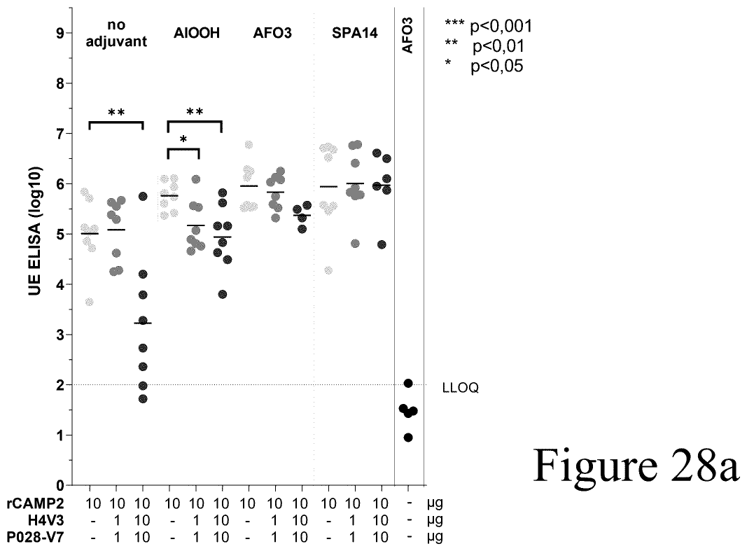

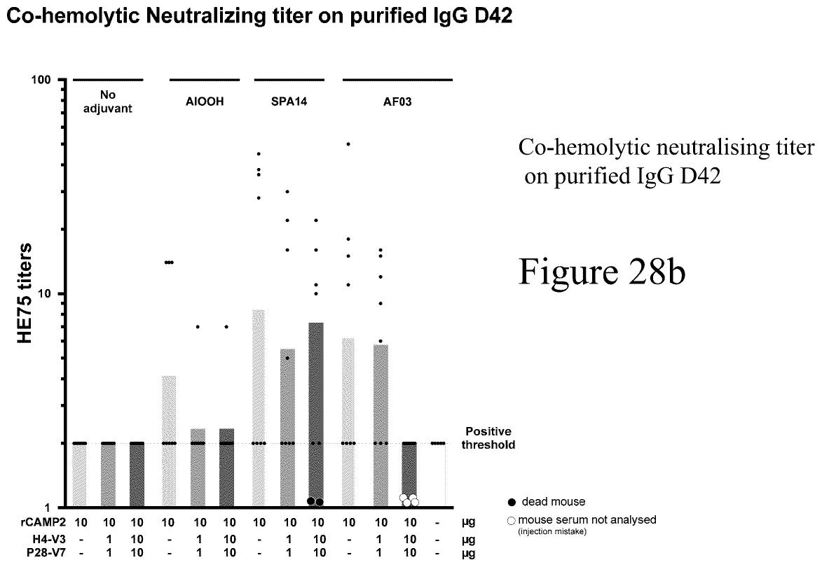

ELISA IgG anti CAMP2 titers from individual sera and co-hemolytic neutralizing titers of IgG purified from individual sera of mice immunized twice at 4 weeks interval with the same recombinant protein antigens as described in FIG. 26 and formulated in the same adjuvants. As shown in FIGS. 28A-28B, interferences were observed for ELISA IgG anti CAMP2 and neutralization titers when H4-V3 and P028-V7 were formulated at 10 μg per dose, both without adjuvant and with AF03. This was also measured at lug and 10 μg doses of H4-V3 and P028-V7 formulated in AlOOH. SPA14 adjuvantation obviated interferences observed in other formulations.

Formulations Containing Combinations of C. acnes Proteins Including Chimeric C. acnes DsA1/DsA2 Polypeptide Give Rise Antibodies which Bind to the Surface of C. acnes, and with Opsonophagocytic Activity

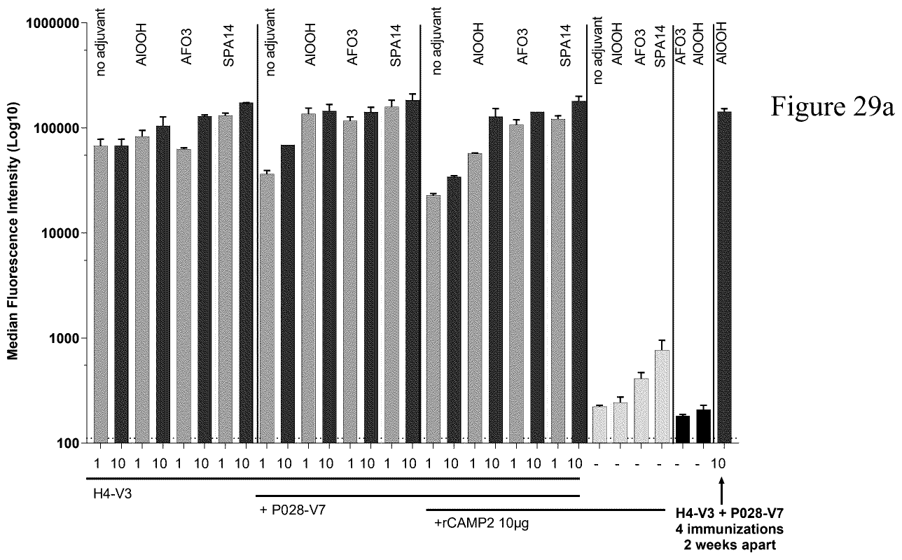

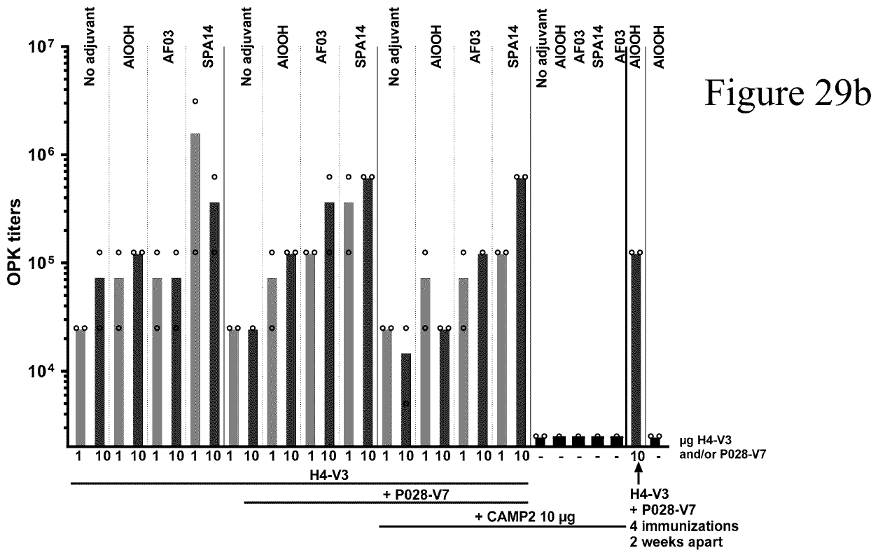

Opsonophagocytic Killing (OPK) titers and Surface Binding (SB) titers were performed on NCTC737 C. acnes strains, from individual sera (SB) and pool of sera (OPK) of mice immunized twice at 4 weeks interval with the same recombinant protein antigens as described in FIG. 26 and formulated in the same adjuvants. As shown in FIGS. 29A-29B, both assays documented comparable or superior titers when the three recombinant proteins were co-formulated at 10 μg of each recombinant protein per dose (bi-valent and tri-valent formulations). At this 10 μg per antigen dose, no overall interference was observed whatever the adjuvant which elicited comparable titers. Lower titers were documented at 1 μg of each recombinant protein per dose, excepted in bivalent formulation at 1 μg formulated in AlOOH. The titers were comparable between groups which received 2 and 4 immunisations, 4 and 2 weeks apart respectively.

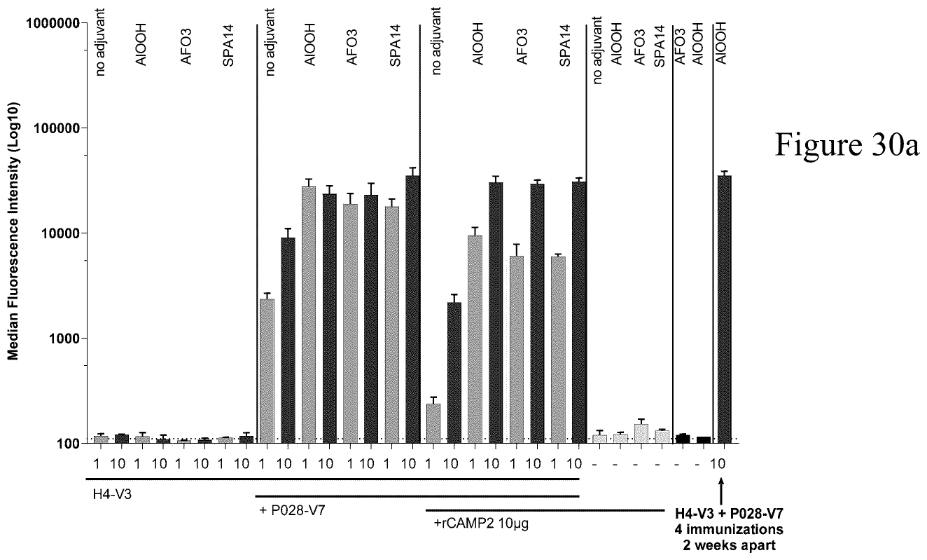

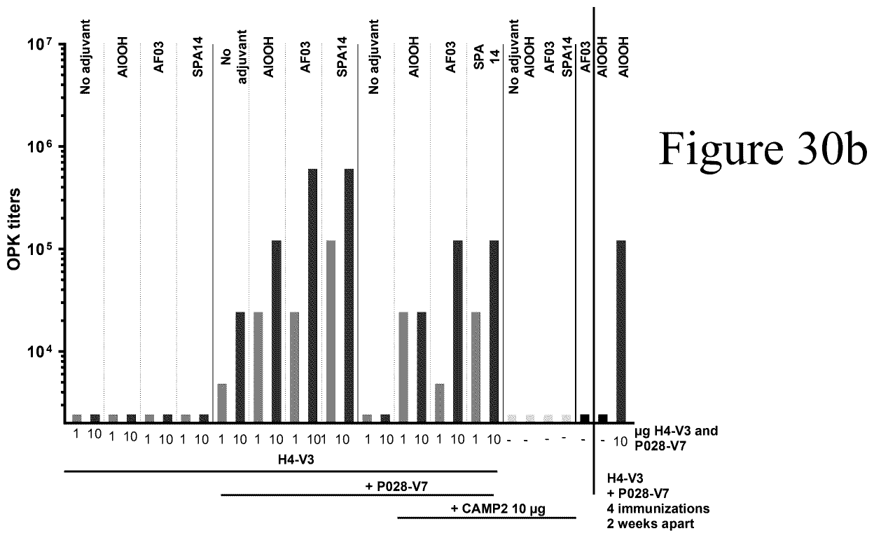

The same experiment was repeated on KPA171202 C. acnes strains, from individual sera (SB) and pool of sera (OPK) of mice. As shown in FIGS. 30A-30B, both assays documented comparable or superior titers when the three recombinant proteins were co-formulated at 10 μg of each recombinant protein per dose (bi-valent and tri-valent formulations). At this 10 μg per antigen dose, no overall interference was observed whatever the adjuvant which elicited comparable titers. Lower titers were documented at lug of each recombinant protein per dose, excepted in bivalent formulation at 1 μg formulated in AlOOH. The titers were comparable between groups which received 2 and 4 immunisations, 4 and 2 weeks apart respectively.

### Example 9—Chimeric C. acnes Antigens

Construct Design of Chimeric C. acnes Antigens

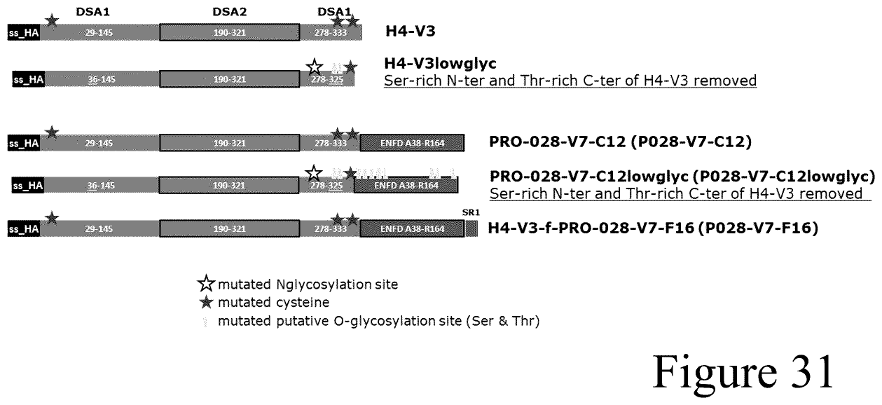

FIG. 31 shows the mRNA constructs coding for chimera proteins that have been designed and synthesized:

Two constructs comprised either an unmutated H4-V3 sequence (SEQ ID NO: 113) or a modified H4-V3 sequence: H4-V3-lowglyc (SEQ ID NO: 123). The latter one was designed to get the lowest probability of glycosylation. H4-V3-lowglyc comprises H4-V3 sequence where the first 7 residues on the N-terminus were deleted. This modified sequence comprised a mutated N-glycosylation site, a truncation of the C-terminal PT-repeat to one copy only, and two additional mutations: S291M and S292G. Western-Blot showed that proteins expressed by HEK cells transfected with H4-V3-lowglyc construct displayed lower molecular weight than H4-V3 indicating that sequence modifications were effective to reduce the level of protein glycosylation.

Three other constructs were designed encoding a triple chimera: Fragments of P028-V7 were fused to H4-V3: either fragment P028-V7-C12 corresponding to ENFD domain (resulting in the chimera of amino acid sequence according to SEQ ID NO: SEQ ID NO: 41) or fragment P028-V7-F16 comprising ENFD domain and part of SRI domain (resulting in the chimera of amino acid sequence according to SEQ ID NO: SEQ ID NO: 38) (see FIG. 20 for schematic visualization of P028 domains). For the triple chimera comprising P028-V7-C12 (the triple chimera has an amino acid sequence corresponding to SEQ ID NO: 41), a counterpart triple chimera construct was designed to get the lowest probability of glycosylation: P028-V7-C12lowglyc (SEQ ID NO: 40). This construct comprised H4-V3-lowglyc, and a GGGGG flexible linker (instead of a PT-repeat) connected to a mutated form of fragment P028-V7-C12. Three of five possible O-glycosylation sites of P028-V7-C12 were mutated to Glycine (Gly). Fragment P028-V7-C12 lacks predicted N-glycosylation sites. Western-Blot showed that proteins expressed by HEK cells transfected with P028-V7-C12lowglyc construct displayed lower molecular weight than P028-V7-C12 indicating that sequence modifications were effective to reduce the level of protein glycosylation.

All these constructs comprised the signal sequence of Influenza Hemagglutinin (HA, H1N1 A/Caledonia/20/1999) (HA SS) at N-ter to address the protein for secretion.

All sequences were derived from the protein sequence of C. acnes strain KPA171202.

Chimeric C. acnes Antigens Elicit Antibodies which Bind the Surface of C. acnes

FIG. 32 shows Surface Binding (SB) titers performed on NCTC737 C. acnes strain, from individual sera of mice immunized twice at 4 weeks interval with the mRNA antigens described in FIG. 31 (“H4-V3” (SEQ ID NO: 113), “H4-V3-lowglyc” (SEQ ID NO: 123), “P028-V7-F16” (SEQ ID NO: 122), “P028-V7-C12” (SEQ ID NO: 125) and “P028-V7-C12lowglyc” (SEQ ID NO: 124)) and with the mRNA constructs coding for P028-V7 (SEQ ID NO: 115). These mRNA constructs were encapsulated in the LNP GL-HEPES-E3-E12-DS-4-E10. The sera were obtained at day 42 following the first immunization. LNP alone was used as negative control. mRNA synthesis was performed using modified nucleotides (in which all uridine nucleosides were replaced with 1-methyl pseudo uridine). The mRNA content in tested formulation was of 1 μg and 5 μg per dose. P028-V7 construct elicited significantly lower SB titer than all others, which induced comparable SB titers at 5 μg dose. At 1 μg dose H4-V3 and H4-V3-lowglyc mRNA induced comparable SB titers which were significantly higher that the triple chimera.

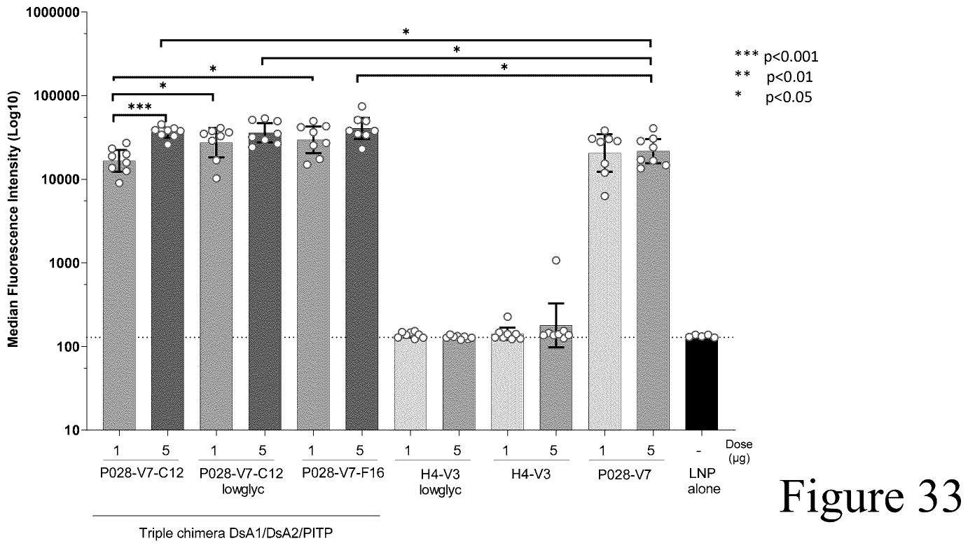

FIG. 33 shows Surface Binding (SB) titers performed on KPA171202 C. acnes strain, from individual sera of mice immunized twice at 4 weeks interval with the same mRNA antigens as described in FIG. 31, and encapsulated in the same LNP. The sera were obtained at day 42 following the first immunization. LNP alone was used as negative control. mRNA synthesis was performed using modified nucleotides (in which all uridine nucleosides were replaced with 1-methyl pseudo uridine). The mRNA content in tested formulation was of 1 μg and 5 μg per dose. All triple chimera constructs were superior to P028-V7 construct at 5 μg mRNA dose. No significant difference could be observed between all triple chimera at 5 μg dose. At lug dose mRNA SB titers were comparable between P028-V7-C12lowglyc and P028-V7-F16, which were significant superior to P028-V7-C12.

Evaluation of Mice after Immunisation with Chimeric C. acnes Antigens

The weight gain with time and the percentage of mice exhibiting swelling at injection site of mice immunized twice at 4 weeks interval with the same mRNA antigens as described in FIG. 31, and encapsulated in the same LNP, was measured. The sera were obtained at day 42 following the first immunization. LNP alone was used as negative control. mRNA synthesis was performed using modified nucleotides (in which all uridine nucleosides were replaced with 1-methyl pseudo uridine).

The mRNA content in tested formulation was of 1 μg and 5 μg per dose. Limited and transient local swelling was observed at the site of injection and no swelling observed after the second injection. Regular weight gain was monitored over the study period for all experimental groups (hence no local reaction and no impact on body weight).

Chimeric C. acnes Antigens Elicit Antibodies which Give Rise to an Anti-DsA1, -DsA2 and -PITP IgG Response

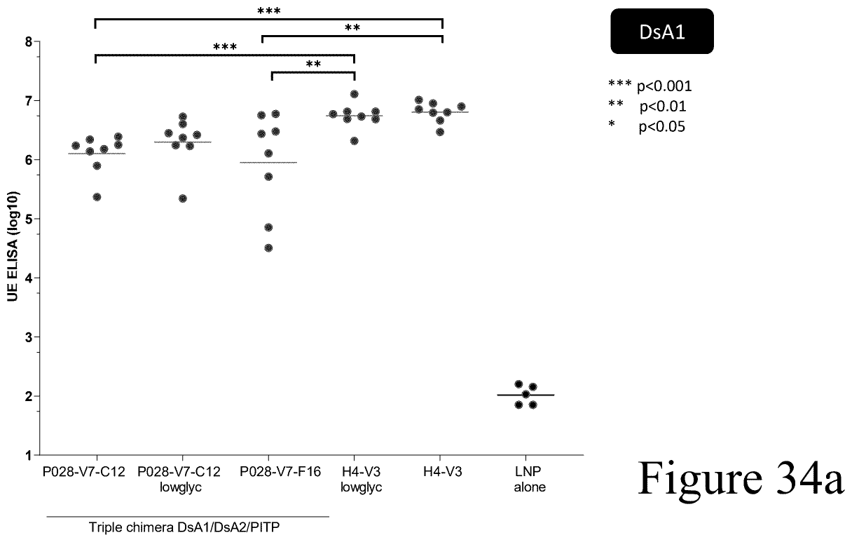

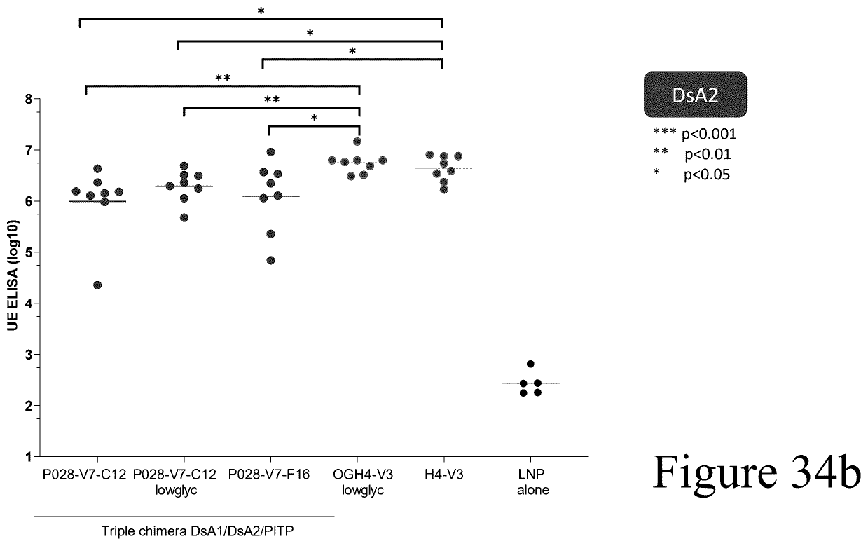

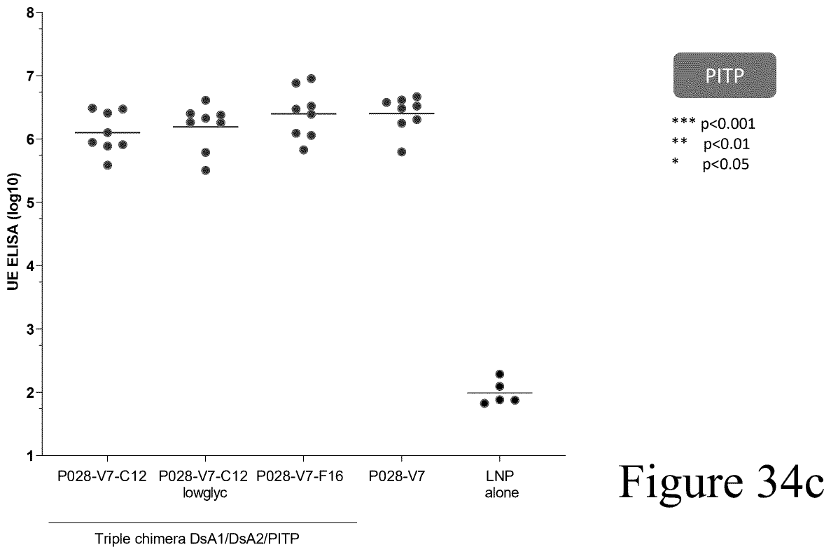

FIGS. 34A-34C show ELISA IgG anti-DsA1, -DsA2 and -PITP titers from individual sera of mice immunized twice at 4 weeks interval with the same mRNA antigens as described in FIG. 31 and encapsulated in the same LNPs. The sera were obtained at day 42 following the first immunization. LNP alone was used as negative control. mRNA synthesis was performed using modified nucleotides (in which all uridine nucleosides were replaced with 1-methyl pseudo uridine). The mRNA content in tested formulation was of 1 μg per dose. The triple chimeras induced lower ELISA IgG anti-DsA1 and -DsA2 titers than the two double chimera H4-V3 and H4-V3-lowglyc. These two double chimeras induced comparable titers. The triple chimeras induced comparable ELISA IgG anti-PITP titers relative to the P028-V7.

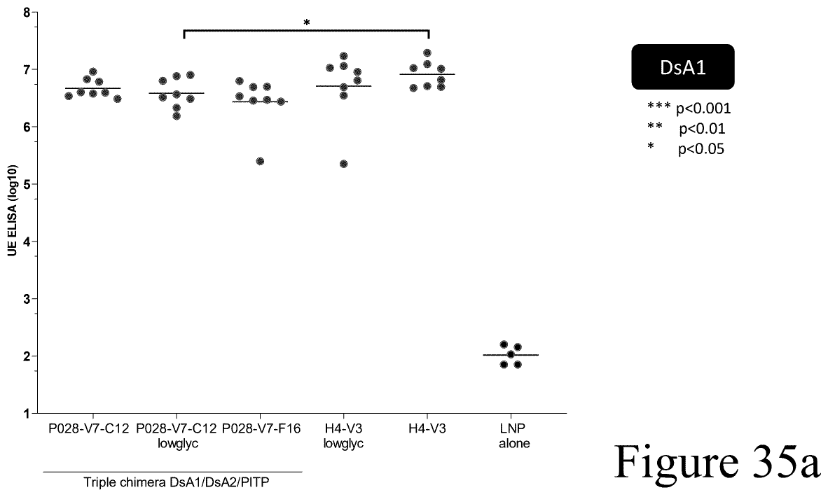

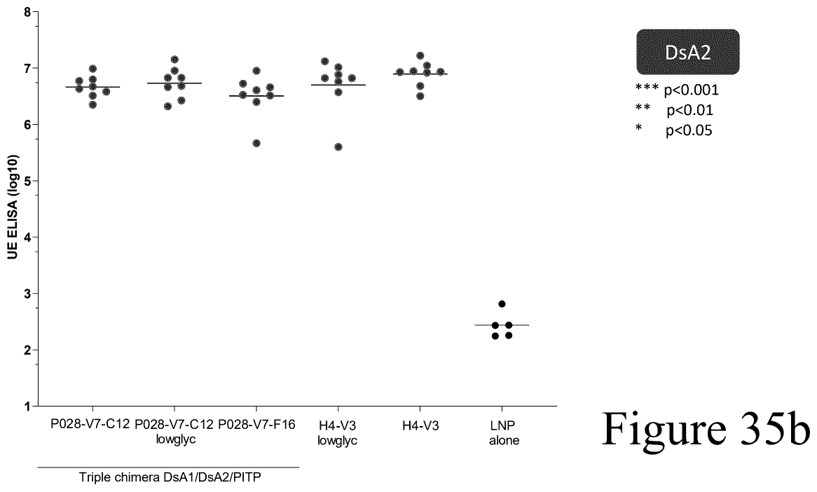

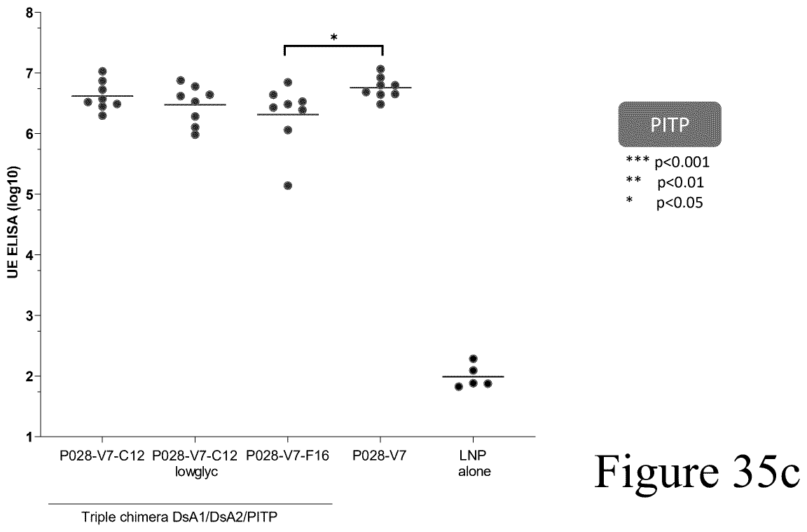

FIGS. 35A-35C show ELISA IgG anti-DSA1, -DSA2 and -PITP titers from individual sera of mice immunized twice at 4 weeks interval with the same mRNA antigens as described in FIG. 31 and encapsulated in the same LNPs. The sera were obtained at day 42 following the first immunization. LNP alone was used as negative control. mRNA synthesis was performed using modified nucleotides (in which all uridine nucleosides were replaced with 1-methyl pseudo uridine). The mRNA content in tested formulation was of 5 μg per dose. All mRNA constructs elicited comparable ELISA IgG anti-DsA1 and -DsA2 titers with exception of H4-V3 mRNA construct which elicited superior response to P028-V7-C12lowglyc one. All mRNA constructs elicited comparable ELISA IgG anti-PITP titers with exception of P028-V7 mRNA construct which elicited superior response to P028-V7-F16 one.

Chimeric C. acnes Antigens Elicit Antibodies which have Opsonophagocytic Activity

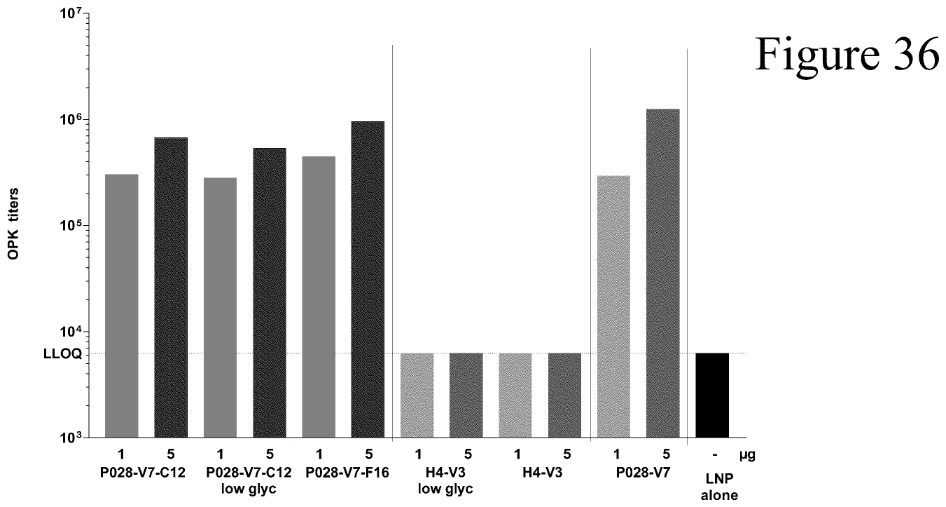

FIG. 36 shows Opsonophagocytic Killing (OPK) titers performed on KPA171202 C. acnes strain, from serum pools of mice immunized twice at 4 weeks interval with the same mRNA antigens as described in FIG. 31, and encapsulated in the same LNP. The sera were obtained at day 42 following the first immunization. LNP alone was used as negative control. mRNA synthesis was performed using modified nucleotides (in which all uridine nucleosides were replaced with 1-methyl pseudo uridine). The mRNA content in tested formulation was of 1 μg and 5 μg per dose. Results illustrate the means of 2 independent experiments. At equivalent dose, tested mRNA constructs elicited comparable OPK titers.

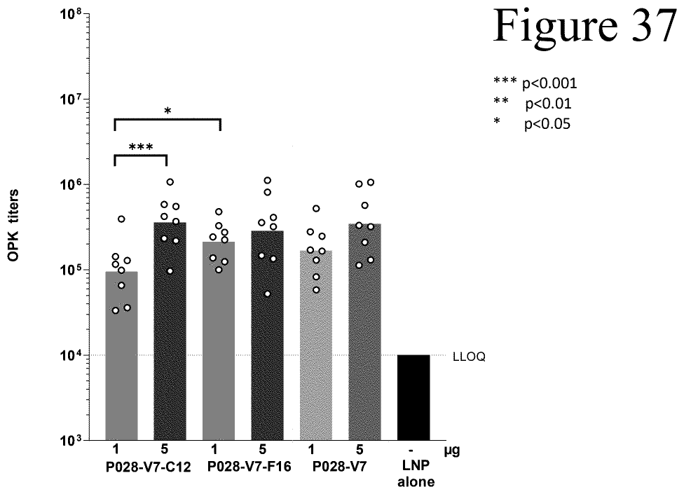

FIG. 37 shows Opsonophagocytic Killing (OPK) titers performed on KPA171202 C. acnes strain, from individual sera of mice immunized twice at 4 weeks interval with the same mRNA antigens as described in FIG. 31, and encapsulated in the same LNP. The graph illustrates the results for P028-V7-F16, P028-V7-C12 and P028-V7 mRNA constructs. The sera were obtained at day 42 following the first immunization. LNP alone was used as negative control. mRNA synthesis was performed using modified nucleotides (in which all uridine nucleosides were replaced with 1-methyl pseudo uridine). The mRNA content in tested formulation was of 1 μg and 5 μg per dose. At equivalent dose, tested mRNA constructs elicited comparable OPK titers.

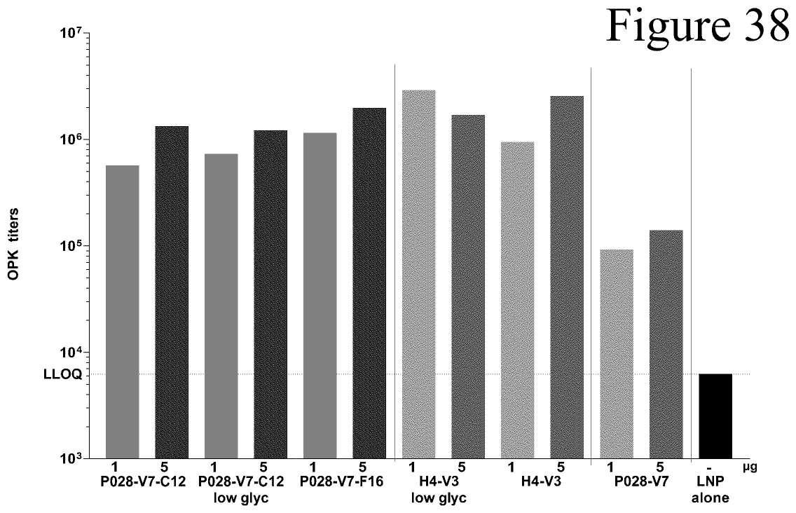

FIG. 38 shows Opsonophagocytic Killing (OPK) titers performed on NCTC737 C. acnes strain, from serum pools of mice immunized twice at 4 weeks interval with the same mRNA antigens as described in FIG. 31, and encapsulated in the same LNP. The sera were obtained at day 42 following the first immunization. LNP alone was used as negative control. mRNA synthesis was performed using modified nucleotides (in which all uridine nucleosides were replaced with 1-methyl pseudo uridine). The mRNA content in tested formulation was of 1 μg and 5 μg per dose. Results illustrate the means of 2 independent experiments. P028-V7 construct elicited significantly lower OPK titer than all others, which induced comparable SB titers at equivalent dose with the exception of H4-V3-lowglyc mRNA construct which seem to induce higher OPK titer than other construct at lug dose.

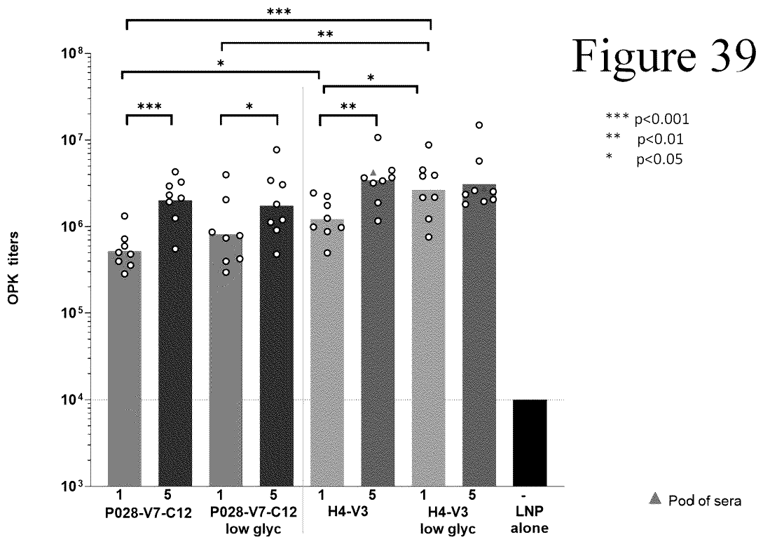

FIG. 39 shows Opsonophagocytic Killing (OPK) titers performed on NCTC737 C. acnes strain, from individual sera of mice immunized twice at 4 weeks interval with the same mRNA antigens as described in FIG. 31, and encapsulated in the same LNP. The graph illustrated the results for P028-V7-C12, P028-V7-C12-lowglyc, H4-V3 and H4-V3-lowglyc mRNA constructs. The sera were obtained at day 42 following the first immunization. LNP alone was used as negative control. mRNA synthesis was performed using modified nucleotides (in which all uridine nucleosides were replaced with 1-methyl pseudo uridine). The mRNA content in tested formulation was of 1 μg and 5 μg per dose. At 5 μg dose, tested mRNA constructs elicited comparable OPK titers. At lug dose, H4-V3-lowglyc mRNA construct elicited higher OPK titers than both P028-V7-C12 and P028-V7-C12-lowglyc, and H4-V3 mRNA construct elicited higher OPK titers than P028-V7-C12.

### Example 10—Comparison of a Chimeric C. acnes DsA1/DsA2/PITP mRNA Antigen Relative to a Combination of a Chimeric C. acnes DsA1/DsA2 mRNA Antigen and of a C. acnes PITP mRNA Antigen

The aim of this example was to compare a ‘triple chimera’ formulation containing an mRNA coding for a chimeric C. acnes DsA1/DsA2/PITP protein (P028-V7-C12), wherein a fragment of P028-V7 corresponding to the ENFD domain, was fused to H4-V3 (as described in Example 9 and FIG. 31), relative to a ‘bivalent combination’ formulation containing a combination of two mRNAs, coding for the H4-V3 and P028-V7 proteins, encapsulated in a LNP, for their ability to induce a functional immune response.

Synthesis and Characterisation of the mRNAs and mRNA-LNP Formulations

mRNA synthesis was performed using modified nucleotides (in which all uridine nucleosides were replaced with 1-methyl pseudo uridine). For the ‘triple chimera’ formulation, an mRNA of SEQ ID NO: 125 coding for the chimera protein P028-V7-C12 of SEQ ID NO: 41, was used. For the ‘bivalent combination’ formulation, a combination of two mRNAs of SEQ ID NO:113 and SEQ ID NO: 115, coding for the proteins H4-V3 of SEQ ID NO: 28 and P028-V7 of SEQ ID NO: 31, respectively, were used. All mRNAs comprised a cap1, a 5′UTR from CMV of SEQ ID NO: 265, a 3′ UTR from hGH of SEQ ID NO: 266, and a polyA tail, and were encapsulated in a LNP containing GL-HEPES-E3-E12-DS-4-E10 as cationic lipid, as described previously. For the ‘bivalent combination’ formulation, the two mRNAs were co-encapsulated in the LNP.

With respect to mRNA size, tail size, % of tailed population and cap1%, all mRNAs met the targeted specifications. Sequencing analyses confirmed that the expected mRNA sequences were generated. With respect to LNP size and % of encapsulation (% EE), all LNPs fell into the expected size range (between 50 and 150 nm) and the targeted % EE (superior to 80%).

### Mouse Immunisations

OF-1 outbred mice (6-week old at day 0) were immunised with the ‘triple chimera’ formulation (at doses ranging between 0.04 μg and 5 μg of mRNA; 8 mice per group) or with the ‘bivalent combination’ formulation (at doses ranging between 0.04 μg and 5 μg for each of the two mRNAs; 8 mice per group), twice, 4 weeks apart (i.e. at day 0 and day 28), through intra-muscular (IM) route (50 μL in quadriceps muscle hind leg, with alternance of the right and left leg between both injections). LNP alone (5 mice per group) was used as negative control. A final bleeding was performed 14 days after the last immunisation (i.e. at day 42 following the first immunisation), and the sera obtained were tested in a total IgG ELISA assay (as described in Example 1), in a Surface Binding assay (as described in Example 1) and/or in an OPK assay (as described in Example 1).

Immunised mice of each experimental group were clinically monitored for local signs, over the study period. There was no impact of the immunisations on body weight or on body temperature. Only a very limited and transient local swelling at the site of injection was observed, with no other clinical reaction.

Chimeric C. acnes DsA1/DsA2/PITP mRNA Antigen and a Combination of a Chimeric C. acnes DsA1/DsA2 mRNA Antigen and a PITP mRNA Antigen Elicit Anti-DsA1, Anti-DsA2 and Anti-PITP IgG Responses

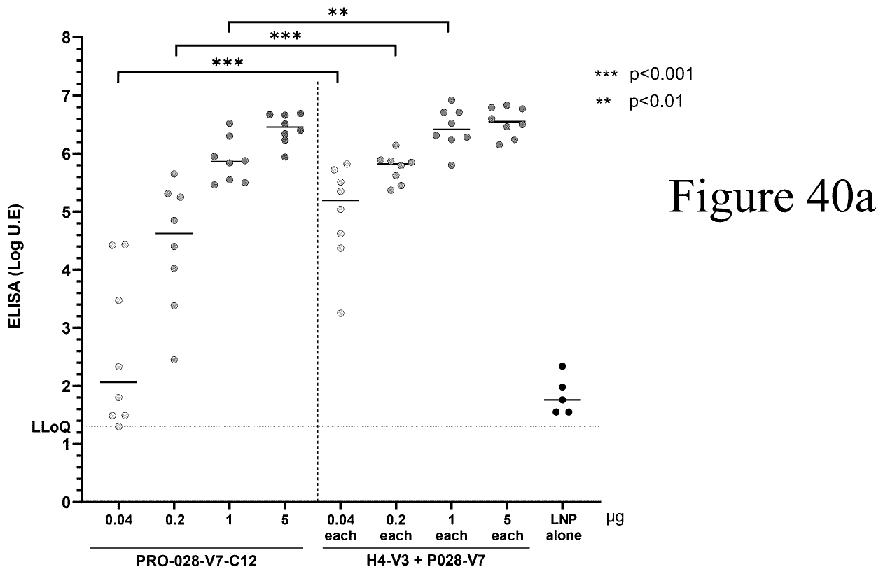

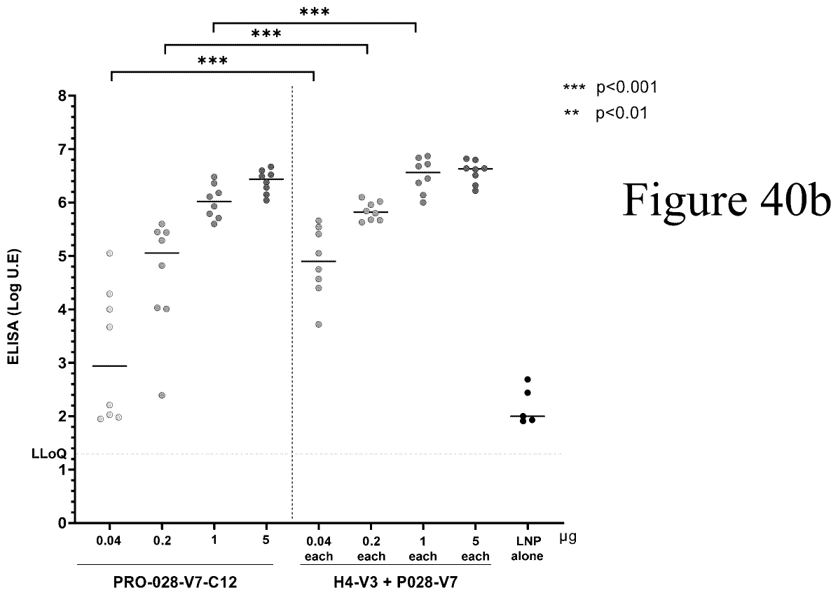

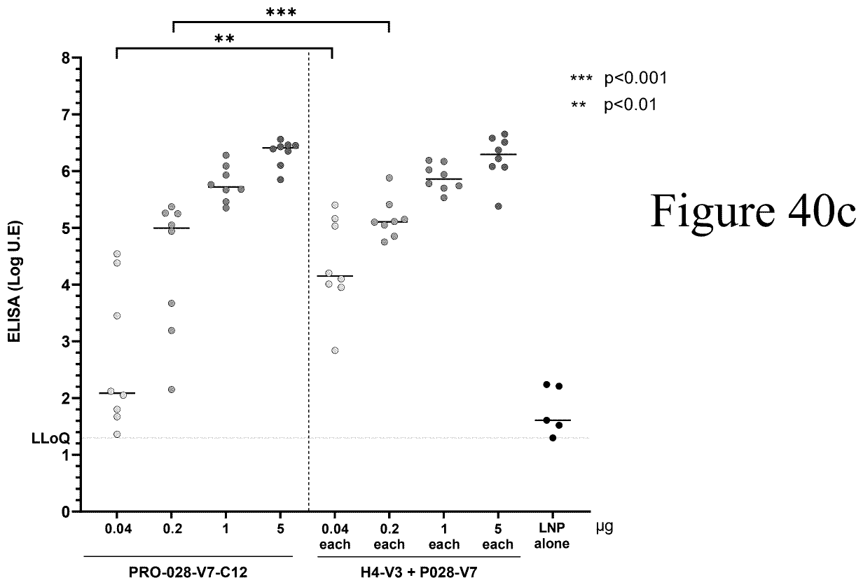

Both the ‘triple chimera’ formulation and the ‘bivalent combination’ formulation elicited anti-DsA1, anti-DsA2 and anti-PITP IgG titers, as measured with an ELISA assay and as shown in FIGS. 40A-40C. At low doses, the ‘triple chimera’ formulation induced lower IgG titers than the ‘bivalent combination’ formulation. A significant dose effect was observed for both formulations. At optimal doses, the ‘triple chimera’ formulation induced similar IgG titers as the ‘bivalent combination’ formulation.

Chimeric C. acnes DsA1/DsA2/PITP mRNA Antigen and a Combination of a Chimeric C. acnes DsA1/DsA2 mRNA Antigen and a PITP mRNA Antigen Elicit Antibodies which Bind to the Surface of C. Acnes

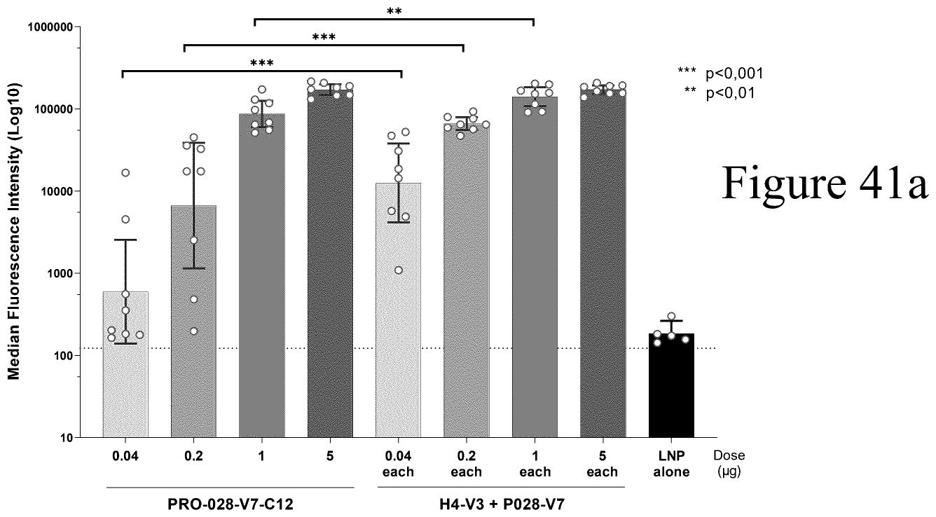

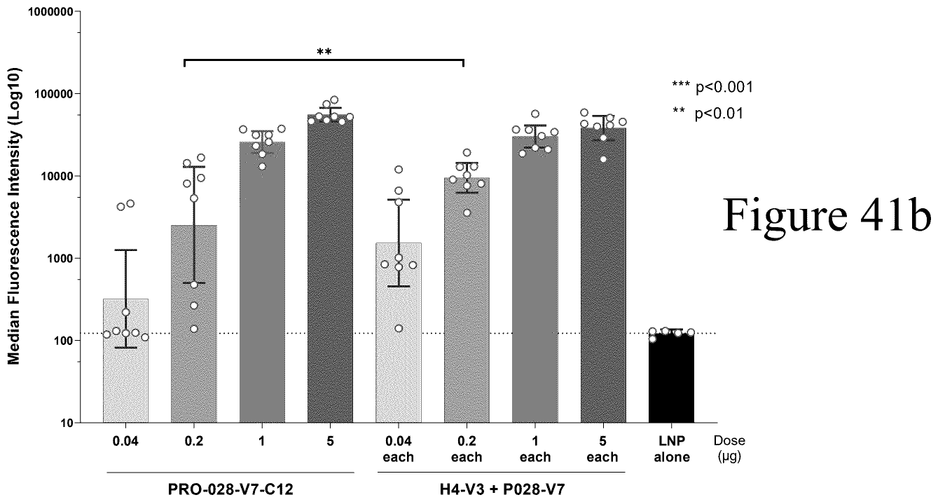

Both the ‘triple chimera’ formulation and the ‘bivalent combination’ formulation elicited SB titers, as measured with a Surface Binding (SB) assay and as shown in FIGS. 41A-41B. At low doses, the ‘triple chimera’ formulation induced lower SB titers than the ‘bivalent combination’ formulation. A significant dose effect was observed for both formulations. At optimal doses, the ‘triple chimera’ formulation induced similar SB titers as the ‘bivalent combination’ formulation.

Chimeric C. acnes DsA1/DsA2/PITP mRNA Antigen and a Combination of a Chimeric C. acnes DsA1/DsA2 mRNA Antigen and a PITP mRNA Antigen Elicit Antibodies with Opsonophagocytic Activity

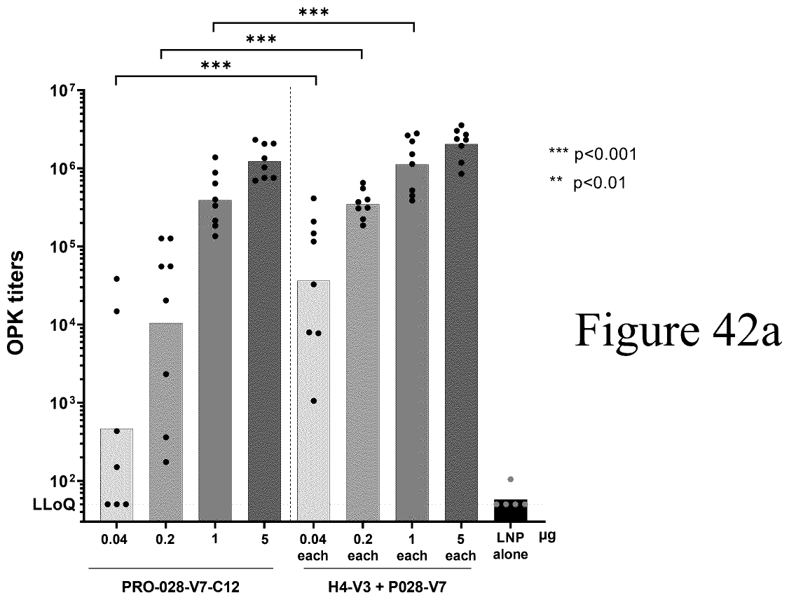

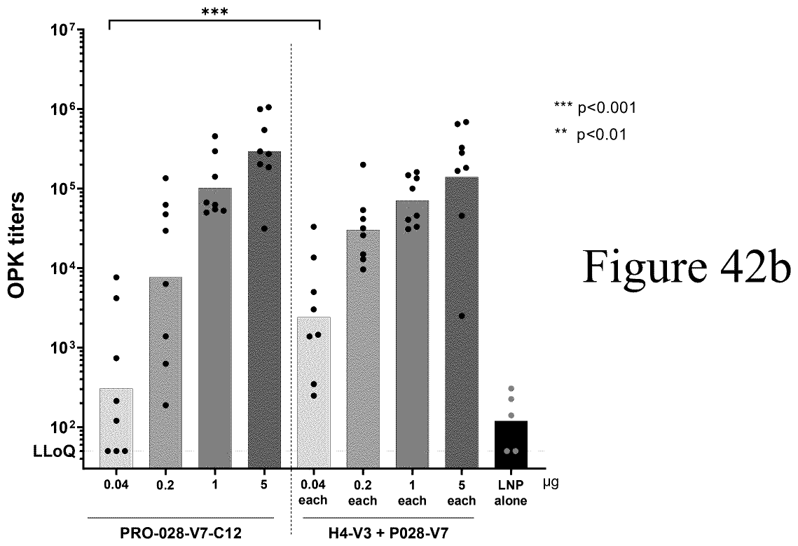

Both the ‘triple chimera’ formulation and the ‘bivalent combination’ formulation elicited antibodies with opsonophagocytic activity, as measured with an Opsonophagocytic Killing (OPK) assay and as shown in FIGS. 42A-42B. At low doses, the ‘triple chimera’ formulation induced lower OPK titers than the ‘bivalent combination’ formulation. A significant dose effect was observed for both formulations. At optimal doses, the ‘triple chimera’ formulation induced similar OPK titers as the ‘bivalent combination’ formulation.

Correlations Between Antibody Responses Elicited by Chimeric C. acnes DsA1/DsA2/PITP mRNA Antigen and a Combination of a Chimeric C. acnes DsA1/DsA2 mRNA Antigen and a PITP mRNA Antigen

The Pearson correlations between ELISA anti-DSA1 and -DSA2 IgG titers, Surface Binding titers and Opsonophagocytic Killing titers performed on NCTC737 C. acnes strain, on one hand, and the Pearson correlations between ELISA anti-PITP IgG titers, Surface Binding titers and Opsonophagocytic Killing titers performed on KPA171202 C. acnes strain, on the other hand, were analysed. All correlated assays exhibited good r values (r=Pearson correlation factor calculated between each variable), and p-values<0.001.

### Example 11—Localisation of Expression Products of C. acnes Antigens with Various Secretion Signal Peptide Sequences and Heterologous Transmembrane Domains in HEK Expi293F Cells

This Example outlines the analysis of cell viability, protein expression, and localization following the transfection of mRNA encoding C. acnes CAMP2 and mRNA encoding C. acnes PITP (see constructs in Table 11) into HEK Expi293F cells.

### Transfection and Western Blot Analysis

For the transfection of cells, HEK Expi293F cells in suspension (5 mL at 2×10⁶ cells/mL—shaker 125 mL) were transfected with 5 μg naked mRNA at 1 μg/μL mixed with equal volume of TransIT-mRNA Reagent and mRNA Boost Reagent-TransIT-mRNA Transfection Kit Mirus (Ref MIR 2250) for 2-5 minutes. The mixture was added to the cells drop-wise and incubated at 37° C., 100 rpm, 8% CO2 for 48 to 72 hours.

HEK Expi293F cell counts and cell percent viability resulting from two expression tests with the mRNA construct panel post-transfection were measured. All cell viability values exceeded 80% at 24 and 48 hours post transfection, indicating that the conditions were normal and that none of the mRNA constructs produced off-target cell cytotoxicity.

Next, mRNA-transfected cells were analysed by Western blot: After transfection, cells and medium were collected and centrifuged (500×g) to collect supernatants. The cell pellets were lysed using Lysozyme (Ready-Lyse Lysozyme Solution-Lucigen ref R1804M)+Benzonase (Sigma-ref E1014)+Protease inhibitor cocktail (Sigma-ref P8340) for 10 min at 20° C. under 800 rpm. The cell pellet lysis was then centrifuged (11,000×g) to collect supernatants and crude extracts.

Extracts from mRNA-transfected HEK293T cells were analyzed by denaturing (95° C.) PAGE using 4-12% Bis-Tris/MES gel (Invitrogen) and Western Blot. Transfer to a nitrocellulose membrane (Bio-Rad) was performed using a semi-dry transfer system (Trans-Blot Turbo Transfer System, Bio-Rad). Blotted proteins were detected with polyclonal antibodies that recognize CAMP2 (rabbit polyclonal antibody, generated in-house, at a 1:1500 dilution) or PITP (mouse polyclonal antibody, generated in house, at a 1:1000 dilution), respectively, and a secondary antibody (anti-rabbit IgG Goat Antibody DyLight 800-Rockland, ref 611-145-002, or anti-mouse IgG). Blots were imaged with Odyssey Infrared Imager—LICOR.

### CAMP2

In vitro expression of CAMP2 fused to signal sequences (labeled “SS” on gels) derived from Influenza A, Influenza B, Rabies, VZV, and Ebola glycoproteins with or without their respective transmembrane domains (“TMB”) was achieved at 48-hours post-transfection in HEK Expi293F cells. CAMP2 (expected size about 26-32 kDa) was detected using a rabbit polyclonal antibody to CAMP2 (generated in-house) at a 1:1500 dilution. Controls included a CAMP2 construct without any SS as well as recombinant CAMP2. With respect to testing CAMP2 localization, samples were collected from crude extracts (total lysate), cell supernatants, and fractionated cell samples containing either intracellular or transmembrane compartments. These results demonstrate that adding a transmembrane domain induces localization of CAMP2 at the cell membrane and reduces secretion and intracellular localization. The Western blot analysis demonstrated that CAMP2 is well expressed and localizes in the expected fractions depending on the presence of a secretion signal peptide or a transmembrane domain.

### PITP

In vitro expression of PITP fused to signal sequences (labeled “SS” on gels) derived from Influenza A, Influenza B, Rabies, VZV, and Ebola glycoproteins with or without their respective transmembrane domains (“TMB”) was achieved at 48-hours post-transfection in HEK Expi293F cells. PITP (expected size about 42-48 kDa) was detected using a mouse polyclonal to PITP (generated in-house) at a 1:1000 dilution. Controls included an PITP construct without any SS or any TMB as well as recombinant PITP. With respect to testing PITP localization, samples were collected from crude extracts (total lysate), cell supernatants, and fractionated cell samples containing either intracellular or transmembrane compartments. Like the CAMP2 antigen localization analysis, these results demonstrate that adding a transmembrane domain induces localization of PITP at the cell membrane and reduces secretion and intracellular localization. Notwithstanding, PITP transmembrane containing constructs did show some escape into the supernatant fraction.

This Western blot analysis demonstrated that PITP is well expressed and localizes in the expected fractions depending on the presence of a secretion signal peptide or a transmembrane domain.

### SUMMARY

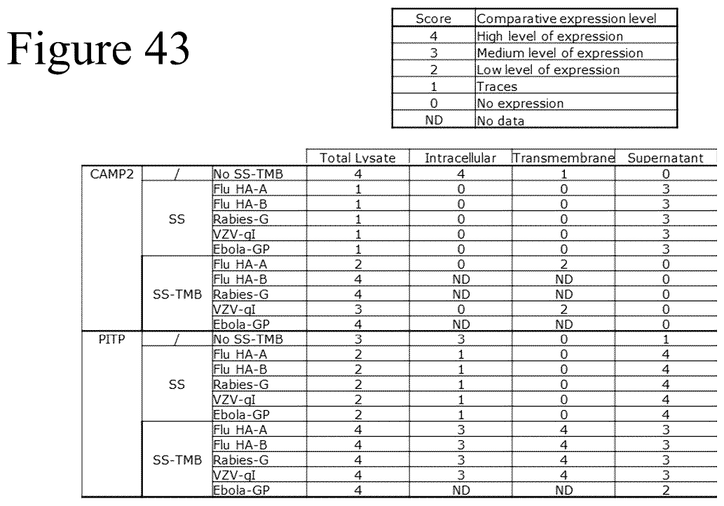

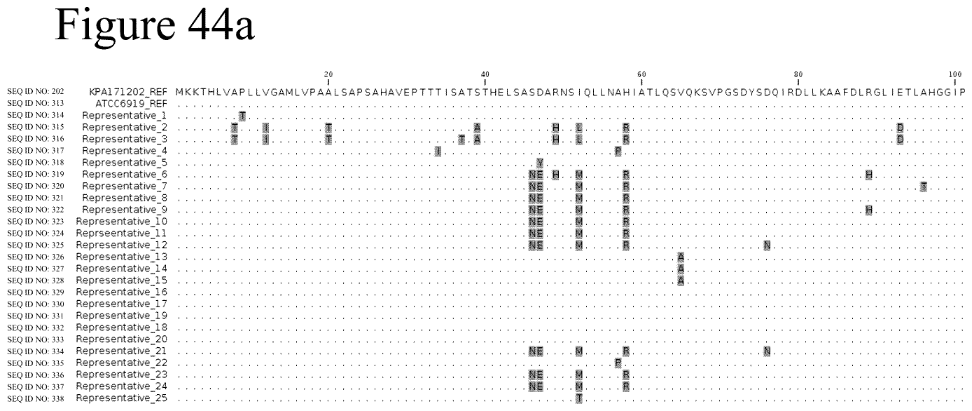

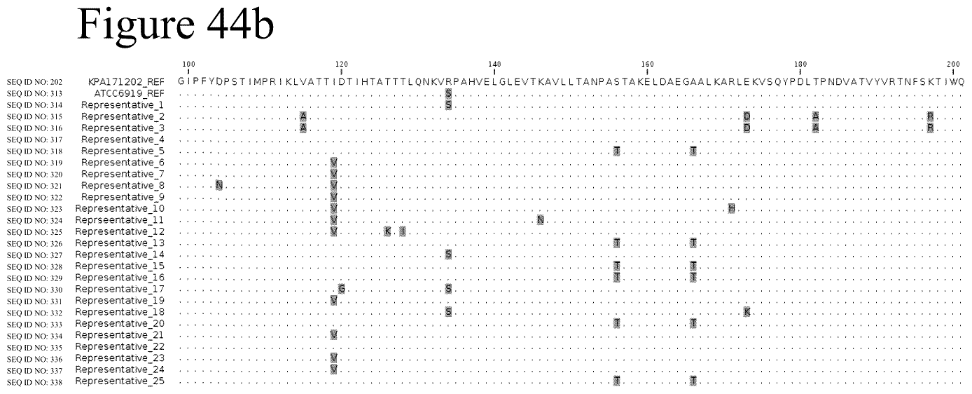

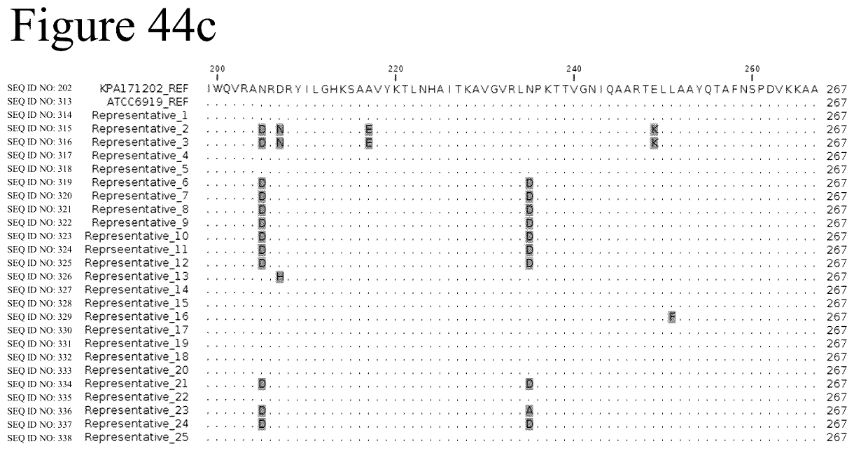

To compare the Western blot analysis for the CAMP2 and PITP, the protein expression and localization results were tabulated as shown in FIG. 43. In general, CAMP2 or PITP containing constructs were expressed in their expected locations but with varying degrees of expression and displayed some escape to the supernatant at varying degrees when the transmembrane domain was introduced.

TABLE 11
Amino Acid Sequence Designs of CAMP2 and PITP Constructs

| | | Encoded by nucleotide |
|---|---|---|
| | Construct | sequence according |
| Description/SEQ ID NO | Name FIG. 43 | to SEQ ID NO: |
| Camp2 | CAMP2 | SEQ ID NO: 289 |
| SEQ ID NO: 1 | No SS-TMB | |
| Camp2_SS_Flu- | CAMP2 | SEQ ID NO: 290 |
| HA_A/California/7/2009 | SS | |
| SEQ ID NO: 268 | Flu HA-A | |
| Camp2_SS_Flu- | CAMP2 | SEQ ID NO: 291 |
| HA_B/Phuket/3073/2013 | SS | |
| SEQ ID NO: 269 | Flu HA-B | |
| Camp2_SS_Rabies- | CAMP2 | SEQ ID NO: 292 |
| G_Pasteur-vaccins | SS | |
| SEQ ID NO: 270 | Rabies-G | |
| Camp2_SS_VZV-gI_Oka- | CAMP2 | SEQ ID NO: 293 |
| vaccine | SS | |
| SEQ ID NO: 271 | VZV-gI | |
| Camp2_SS_Ebola- | CAMP2 | SEQ ID NO: 294 |
| GP_Mayinga-76 | SS | |
| SEQ ID NO: 272 | Ebola-GP | |
| Camp2_SS_Flu- | CAMP2 | SEQ ID NO: 295 |
| HA_A/California/7/2009_ | SS-TMB | |
| TMB | Flu HA-A | |
| SEQ ID NO: 273 | | |
| Camp2_SS_Flu- | CAMP2 | SEQ ID NO: 296 |
| HA_B/Phuket/3073/2013_ | SS-TMB | |
| TMB | Flu HA-B | |
| SEQ ID NO: 274 | | |
| Camp2_SS_Rabies- | CAMP2 | SEQ ID NO: 297 |
| G_Pasteur-vaccins_TMB | SS-TMB | |
| SEQ ID NO: 275 | Rabies-G | |
| Camp2_SS_VZV-gI_Oka- | CAMP2 | SEQ ID NO: 298 |
| vaccine_TMB | SS-TMB | |
| SEQ ID NO: 276 | VZV-gI | |
| Camp2_SS_Ebola- | CAMP2 | SEQ ID NO: 299 |
| GP_Mayinga-76_TMB | SS-TMB | |
| SEQ ID NO: 277 | Ebola-GP | |
| PITP | PITP | SEQ ID NO: 300 |
| SEQ ID NO: 278 | No SS-TMB | |
| PITP_SS_Flu- | PITP | SEQ ID NO: 301 |
| HA_A/California/7/2009 | SS | |
| SEQ ID NO: 279 | Flu HA-A | |
| PITP_SS_Flu- | PITP | SEQ ID NO: 302 |
| HA_B/Phuket/3073/2013 | SS | |
| SEQ ID NO: 280 | Flu HA-B | |
| PITP_SS_Rabies- | PITP | SEQ ID NO: 303 |
| G_Pasteur-vaccins | SS | |
| SEQ ID NO: 281 | Rabies-G | |
| PITP_SS_VZV-gI_Oka- | PITP | SEQ ID NO: 304 |
| vaccine | SS | |
| SEQ ID NO: 282 | VZV-gI | |
| PITP_SS_Ebola- | PITP | SEQ ID NO: 305 |
| GP_Mayinga-76 | SS | |
| SEQ ID NO: 283 | Ebola-GP | |
| PITP_SS_Flu- | PITP | SEQ ID NO: 306 |
| HA_A/California/7/2009_ | SS-TMB | |
| TMB | Flu HA-A | |
| SEQ ID NO: 284 | | |
| PITP_SS_Flu- | PITP | SEQ ID NO: 307 |
| HA_B/Phuket/3073/2013_ | SS-TMB | |
| TMB | Flu HA-B | |
| SEQ ID NO: 285 | | |
| PITP_SS_Rabies- | PITP | SEQ ID NO: 308 |
| G_Pasteur-vaccins_TMB | SS-TMB | |
| SEQ ID NO: 286 | Rabies-G | |
| PITP_SS_VZV-gI_Oka- | PITP | SEQ ID NO: 309 |
| vaccine_TMB | SS-TMB | |
| SEQ ID NO: 287 | VZV-gI | |
| PITP_SS_Ebola- | PITP | SEQ ID NO: 310 |
| GP_Mayinga-76_TMB | SS-TMB | |
| SEQ ID NO: 288 | Ebola-GP | |

### Example 12—Synthesis of IM-001 According to Scheme 2

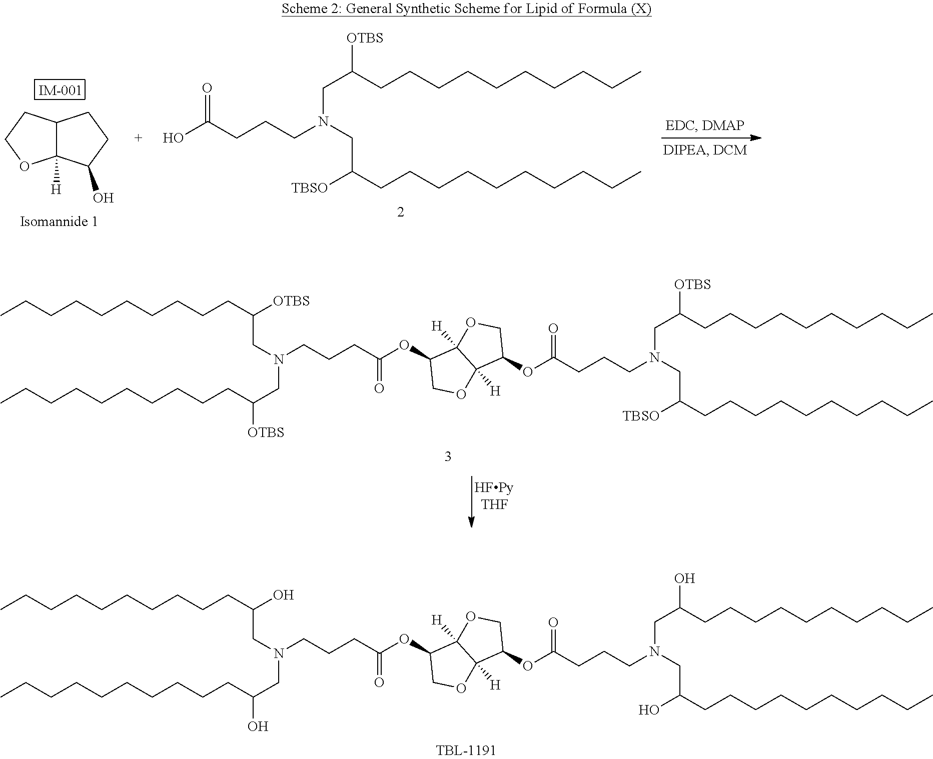

### Abbreviations:

DCM: Dichloromethane
DIPEA: N,N-Diisopropylethylamine
DMAP: 4-Dimethylaminopyridine
EDC: 1-Ethyl-3-(3-dimethylaminopropyl) carbodiimide
EtOAc: Ethyl acetate
NaHCO₃: Sodium hydrogencarbonate
Py: Pyridine
Na₂SO4: Sodium Sulfate
TEA: Triethylamine
TFA: Trifluoroacetic Acid
MS: Mass spectrometry
ESI-MS: Electrospray ionization mass spectrometry
TLC: Thin Layer Chromatography

### Step 1: Synthesis of Intermediate (3)

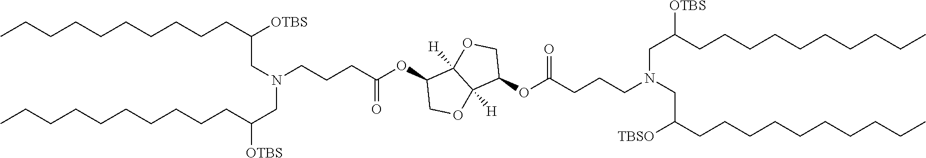 [SMILES: CCCCCCCCCCC(CN(CCCC(=O)O[C@@H]1CO[C@H]2[C@@H]1OC[C@H]2OC(=O)CCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 2: To a solution of acid (2) (4.58 g, 6.55 mmol) and isomannide (1) (0.38 g, 2.62 mmol) in dichloromethane (40 mL) were added DIPEA (3.65 mL, 20.96 mmol), DMAP (0.32 g, 2.62 mmol) and EDC (1.5 g, 7.86 mmol). The resulting mixture was stirred at room temperature for overnight. After 16 h, MS and TLC (30% EtOAc in hexanes) analysis indicated completion of the reaction. The reaction mixture was diluted with dichloromethane and washed with saturated NaHCO₃ solution, water and brine solution. The organic layer was dried over anhydrous Na₂SO4 and concentrated. The crude residue was purified, and the desired product was eluted at 6% EtOAc in hexanes. The product containing fractions were concentrated to obtain 2.58 g (65%) of pure product.

### Results:

ESI-MS: Calculated C₈₆H₁₇₇N₂O₁₀Si₄, [M+H⁺]=1510.25, Observed=1510.3

### Step 2: Synthesis of IM-001

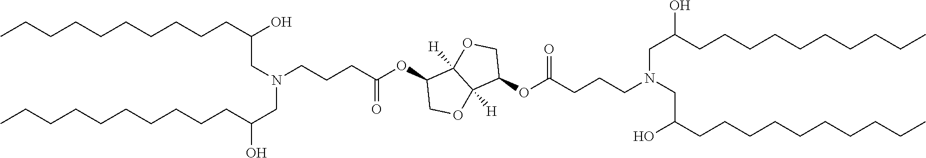 [SMILES: CCCCCCCCCCC(O)CN(CCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCCCCCC]

As depicted in Scheme 2: To a solution of Intermediate (3) (2.58 g, 1.70 mmol) in tetrahydrofuran (14 mL) was added hydrogen fluoride (70% HF.py complex, 7 mL, 51.23 mmol) at 0° C. and stirred at the same temperature for 5 minutes. Then reaction mixture was warmed to room temperature and stirred for 16 h. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water and brine. Then dried over anhydrous Na₂SO4 and concentrated. The crude residue was purified, and the desired product was eluted at 67% EtOAc in hexanes. The purest fractions were concentrated to obtain 1.1 g (61%) of pure product.

### Results:

¹H NMR (400 MHZ, CDCl₃) δ 5.13-5.03 (m, 2H), 4.73-4.65 (m, 2H), 4.29-3.83 (m, 8H), 3.52-2.98 (m, 12H), 2.69-2.49 (m, 4H), 2.32-2.09 (m, 4H), 1.73-1.12 (m, 72H), 0.88 (t, J=6.6 Hz, 12H).

ESI-MS: Calculated C₆₂H₁₂₁N₂O₁₀, [M+H⁺]=1053.90, Observed=1053.2 and 527.2 [M/2+H⁺].

### Example 13—Chimeric C. acnes DsA1/DsA2/PITP/CAMP2 mRNA Antigens

The aim of this example was to assess formulations containing chimeric C. acnes DsA1/DsA2/PITP/CAMP2 mRNA constructs (i.e, wherein the four antigens are located on the same mRNA molecule) for their ability to induce a functional immune response, and to compare them with formulations containing a ‘bivalent’ combination of a CAMP2 mRNA construct and a chimeric C. acnes DsA1/DsA2/PITP mRNA construct.

Design of Chimeric C. acnes DsA1/DsA2/PITP/CAMP2 Constructs

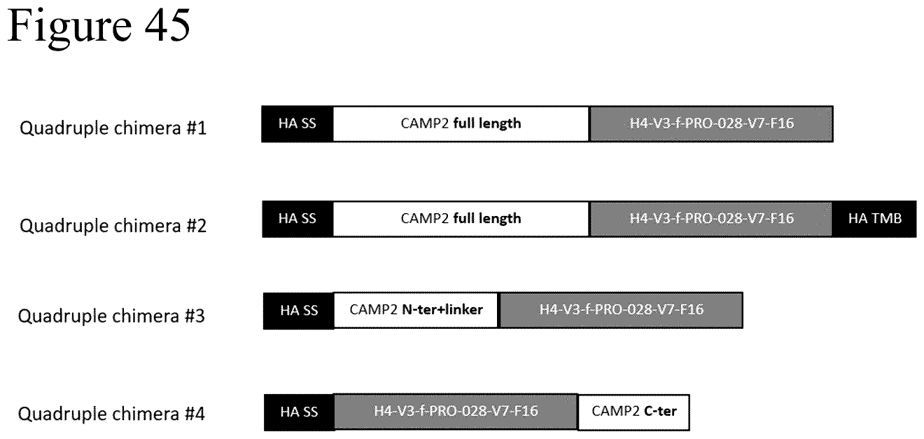

FIG. 45 shows the ‘quadruple chimera’ C. acnes DsA1/DsA2/PITP/CAMP2 constructs which have been designed and tested in the following experiments:
A quadruple chimera (‘Quadruple chimera #1’), comprising a full length CAMP2 at N-ter of triple chimera H4-V3-f-PRO-028-V7-F16, with a signal sequence of Influenza Hemagglutinin (HA, H1N1 A/Caledonia/20/1999) (HA SS) at N-ter of the quadruple chimera to address the protein for secretion,
A quadruple chimera (‘Quadruple chimera #2’), comprising a full length CAMP2 at N-ter of triple chimera H4-V3-f-PRO-028-V7-F16, with a HA SS at N-ter and a transmembrane domain of Influenza Hemagglutinin (HA, H1N1 A/Caledonia/20/1999) (HA TMB) at C-ter of the quadruple chimera to anchor the protein at the cell membrane,
A quadruple chimera (‘Quadruple chimera #3’), comprising the N-ter and linker portions of CAMP2 at N-ter of triple chimera H4-V3-f-PRO-028-V7-F16, with a HA SS at N-ter of the quadruple chimera to address the protein for secretion,
A quadruple chimera (‘Quadruple chimera #4’) comprising the C-ter portion of CAMP2 at C-ter of triple chimera H4-V3-f-PRO-028-V7-F16, with a HA SS at N-ter of the quadruple chimera to address the protein for secretion.
   Characterization of the Expression and Localization of Chimeric C. acnes DsA1/DsA2/PITP/CAMP2 mRNA Constructs in HEK Cells

For this experiment, the mRNAs used were as follows:
Two codon-optimized mRNAs encoding a quadruple chimera comprising SEQ ID NO: 374 (Quadruple chimera #1):
   Quadruple chimera #1 (CO1) of SEQ ID NO: 379 and
   Quadruple chimera #1 (CO2) of SEQ ID NO: 380 encoding the quadruple chimera of SEQ ID NO: 370
Two codon-optimized mRNAs encoding the quadruple chimera of SEQ ID NO: 369 (Quadruple chimera #2):
   Quadruple chimera #2 (CO1) of SEQ ID NO: 377 and
   Quadruple chimera #2 (CO2) of SEQ ID NO: 378
Two codon-optimized mRNAs encoding the quadruple chimera of SEQ ID NO: 371 (Quadruple chimera #3):
   Quadruple chimera #3 (CO1) of SEQ ID NO: 381 and
   Quadruple chimera #3 (CO2) of SEQ ID NO: 382
One codon-optimized mRNA encoding the quadruple chimera of SEQ ID NO: 372 (Quadruple chimera #4):
   Quadruple chimera #4 (CO2) of SEQ ID NO: 383
As controls: one mRNA of SEQ ID NO: 113 encoding H4-V3, one mRNA of SEQ ID NO: 115 encoding P028-V7, one mRNA of SEQ ID NO: 91 encoding CAMP2-TMB and one mRNA of SEQ ID NO: 125 encoding the PRO-028-V7-C12 triple chimera.

Each mRNA comprised a cap1, a 5′UTR from CMV of SEQ ID NO: 265, a 3′ UTR from hGH of SEQ ID NO: 266, and a polyA tail. mRNA synthesis was performed using modified nucleotides (in which all uridine nucleosides were replaced with 1-methyl pseudo uridine), and the mRNA constructs were transfected into HEK cells (in vitro cell transfection).

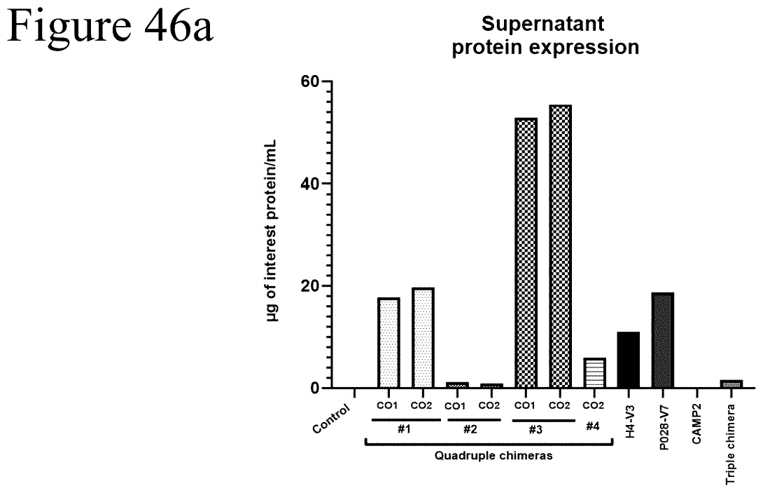

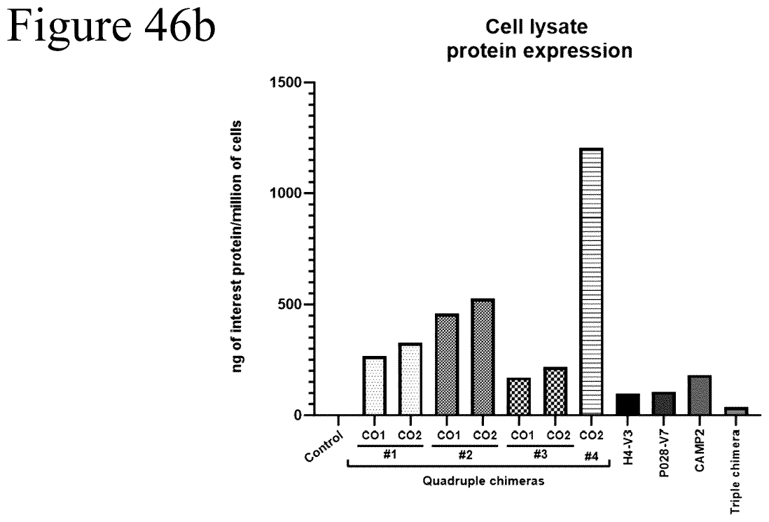

Protein expression was then assessed and measured in the supernatant and in the total cell lysate by HPLC-MS. As shown in FIGS. 46A-46B, all constructs were expressed in their expected compartments. In particular, the quadruple chimera #2 with a transmembrane domain was found in the total cell lysate, but not in the supernatant. The three other quadruple chimeras, designed to be secreted, were found both in the supernatant and in the total cell lysate. The quadruple chimera #3 with CAMP2 N-ter and linker portions was found to be the most highly secreted. The quadruple chimera #4 with a CAMP2 C-ter portion was found to be secreted at the lowest levels.

Similar results were obtained by Western blot (using as primary antibodies: an anti-CAMP2 rabbit polyclonal antibody, an anti-DsA2 mouse monoclonal antibody or an anti-PITP mouse monoclonal antibody), with all quadruple chimera proteins being expressed in their expected compartment (cell lysate and/or supernatant).

### Immunization of Mice

Constructs were then tested for their ability to elicit functional antibodies in mice. In these experiments, the following mRNA constructs were used:
One mRNA of SEQ ID NO: 380 encoding the Quadruple chimera #1
One mRNA of SEQ ID NO: 378 encoding the Quadruple chimera #2
One mRNA of SEQ ID NO: 382 encoding the Quadruple chimera #3
One mRNA of SEQ ID NO: 383 encoding the Quadruple chimera #4
For comparison: a “bivalent combination” of one mRNA of SEQ ID NO: 91 encoding a CAMP2-TMB and one mRNA of SEQ ID NO: 122 encoding the H4-V3-f-PRO-028-V7-F16 triple chimera.

Each mRNA comprised a cap1, a 5′UTR from CMV of SEQ ID NO: 265, a 3′ UTR from hGH of SEQ ID NO: 266, and a polyA tail. mRNA synthesis was performed using modified nucleotides (in which all uridine nucleosides were replaced with 1-methyl pseudo uridine). The mRNA constructs were encapsulated in an LNP composed of four lipids: GL-HEPES-E3-E12-DS-4-E10 (as the ionizable lipid)/DOPE/cholesterol/DMG-PEG, at the molar ratio of 40:30:28.5:1.5. LNP alone served as a negative control.

OF-1 outbred mice (6-week old at day 0) were immunized twice at 4 weeks interval through intra-muscular (IM) route (50 μL in quadriceps muscle hind leg, with alternance of the right and left leg between both injections). The mRNA content in tested formulation was of 1 μg or 5 μg per dose (for the quadruple chimeras) and of [1 μg+1 μg] or [5 μg+5 μg] per dose (for the combination of CAMP2-TMB and triple chimera).

Mice were monitored for clinical signs. Only a limited and transient local swelling was observed at the site of injection following the second injection in all experimental groups, with no other clinical reaction (such as straight hair, loss of mobility, apathy . . . ). Moreover, regular weight gain was monitored over the study period for all experimental groups. Overall, there was no significant impact of the injections of the different formulations on clinical signs.

The sera were obtained at day 42 following the first immunization, and tested for total IgG by ELISA, in an OPK assay, in a Surface Binding assay and in a co-hemolysis neutralization assay (as described in Example 1). Results are presented below.

Chimeric C. acnes DsA1/DsA2/PITP/CAMP2 mRNA Constructs and a Bivalent Combination of a CAMP2-TMB mRNA Construct and a DsA1/DsA2/PITP mRNA Construct Elicit Anti-DsA1, Anti-DsA2, Anti-PITP and Anti-CAMP 2 IgG Responses

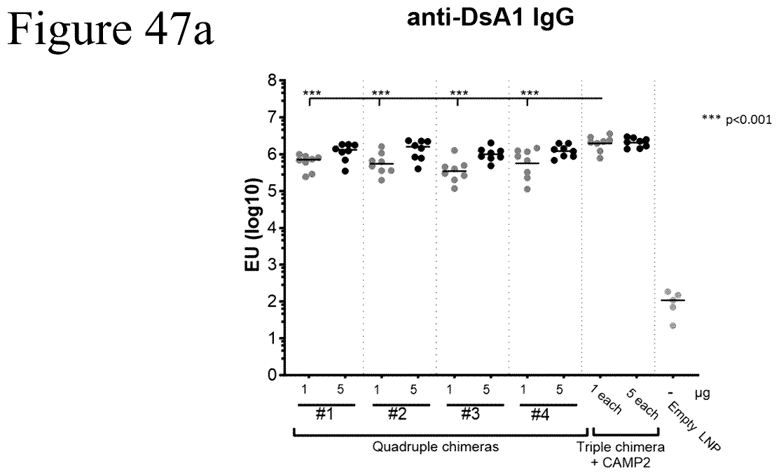

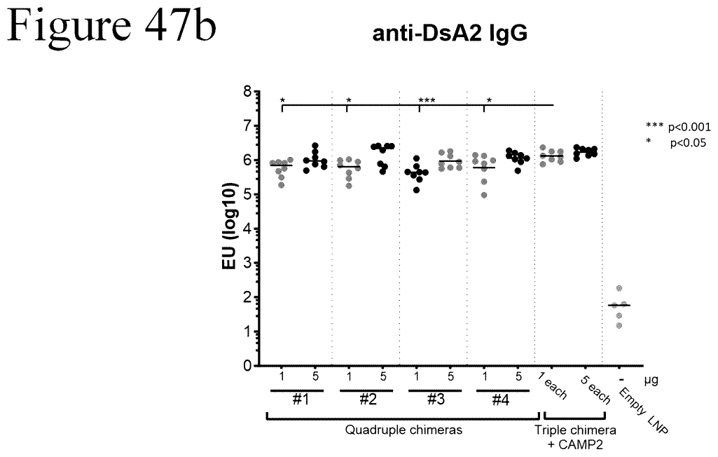

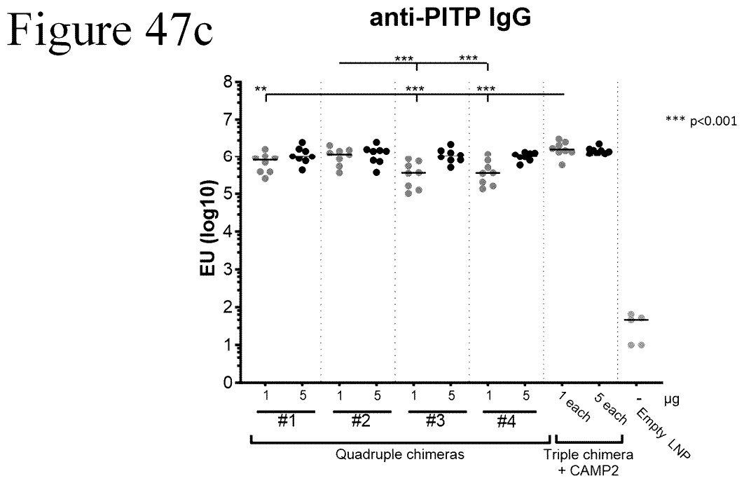

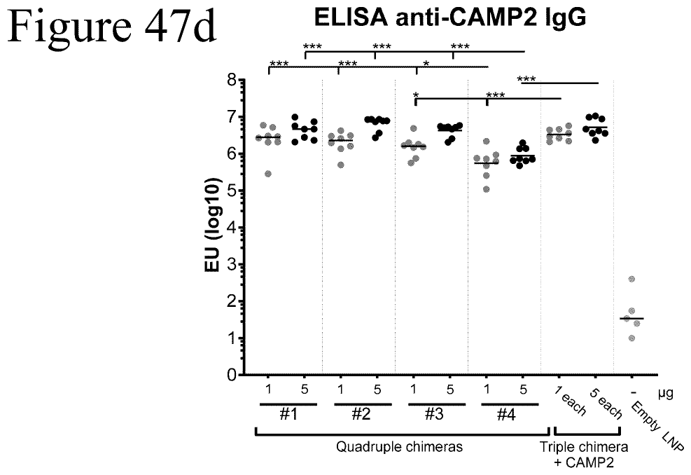

Both the “quadruple chimera” formulations and the “bivalent combination” formulation elicited anti-DsA1, anti-DsA2, anti-PITP and anti-CAMP2 IgG responses, as shown in FIGS. 47A-47D.

At equivalent dose, all quadruple chimeras elicited comparable anti-DsA1 (FIG. 47a) and anti-DsA2 (FIG. 47b) IgG titers. At lug dose, anti-DsA1 and anti-DsA2 Ig titers were significantly lower than the bivalent combination. At 5 μg dose all quadruple chimeras induced similar IgG titers as the bivalent combination. There was a significant difference between the 1 μg and 5 μg doses for all groups, except for the quadruple chimera #1 and the bivalent combination.

As shown in FIG. 47c, at lug dose, anti-PITP Ig titers induced by quadruple chimeras were significantly lower than the bivalent combination, except for the quadruple chimera #2. At this lug dose, the quadruple chimera #2 elicited an anti-PITP IgG titer equivalent to the quadruple chimera #1 and significantly superior to the quadruple chimeras #3 and #4. At 5 μg dose, all quadruple chimeras elicited equivalent anti-PITP IgG titers compared to the bivalent combination.

As shown in FIG. 47d, at lug dose, the bivalent combination elicited anti-CAMP2 Ig titers comparable to the ones elicited by quadruple chimeras #1 and #2 and significantly higher than the ones elicited by quadruple chimeras #3 and #4. At lug, the anti-CAMP2 Ig titer elicited by the quadruple chimera #4 was significantly lower than all tested formulations. At 5 μg dose, all quadruple chimeras elicited comparable anti-CAMP2 IgG titers to the bivalent combination, except the quadruple chimera #4 for which IgG titers were lower.

Chimeric C. acnes DsA1/DsA2/PITP/CAMP2 mRNA Constructs and a Bivalent Combination of a CAMP2-TMB mRNA Construct and a DsA1/DsA2/PITP mRNA Construct Elicit Antibodies which have Opsonophagocytic Activity

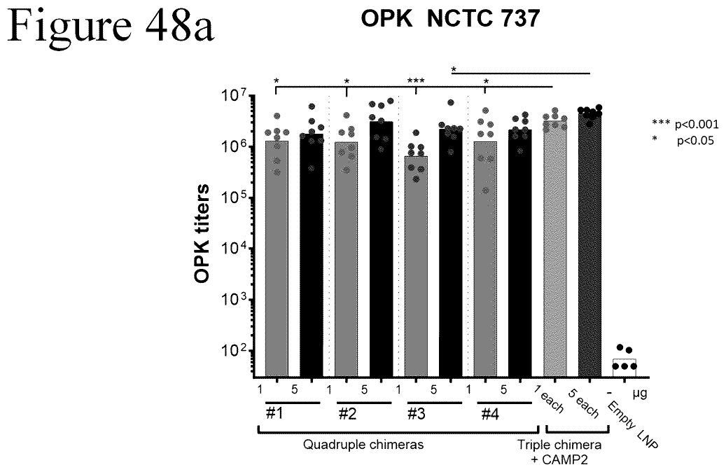

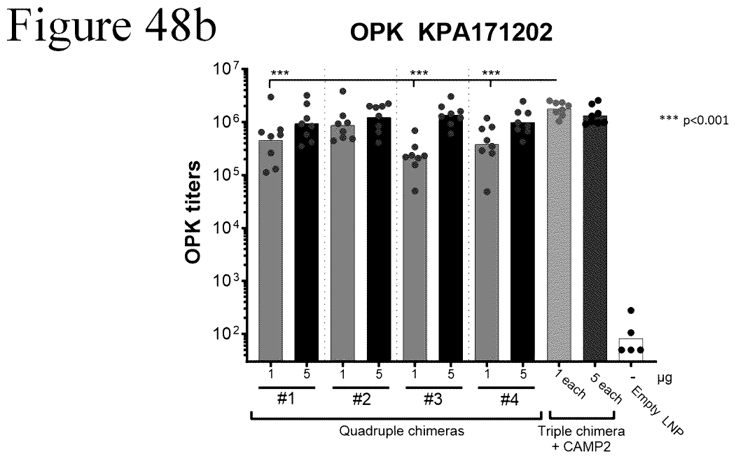

Both the “quadruple chimera” formulations and the “bivalent combination” formulation elicited antibodies with opsonophagocytic activity, as measured with an Opsonophagocytic Killing (OPK) assay and as shown in FIGS. 48A-48B.

All four quadruple chimeras performed similarly in the OPK assay on C. acnes strain NCTC737 (FIG. 48a). There was a significant difference between the 1 μg and 5 μg doses for all groups, except for the quadruple chimera #1 and the bivalent combination. At the lug dose, all quadruple chimeras induced comparable OPK titers, which were lower than the ones with the bivalent combination. At the 5 μg dose, all quadruple chimeras (except quadruple chimera #3) induced similar OPK titers as the bivalent combination.

All four quadruple chimeras performed similarly in the OPK assay on C. acnes strain KPA171202 (FIG. 48b). There was a significant difference between the 1 μg and 5 μg doses for quadruple chimera #3 and #4. At the lug dose, the quadruple chimeras induced lower OPK titers than the bivalent combination, except quadruple chimera #2 for which OPK titers were equivalent. At the 5 μg dose, all quadruple chimeras induced similar OPK titers as the bivalent combination.

Chimeric C. acnes DsA1/DsA2/PITP/CAMP2 mRNA Constructs and a Bivalent Combination of a CAMP2-TMB mRNA Construct and a DsA1/DsA2/PITP mRNA Construct Elicit Antibodies which Bind to the Surface of C. acnes

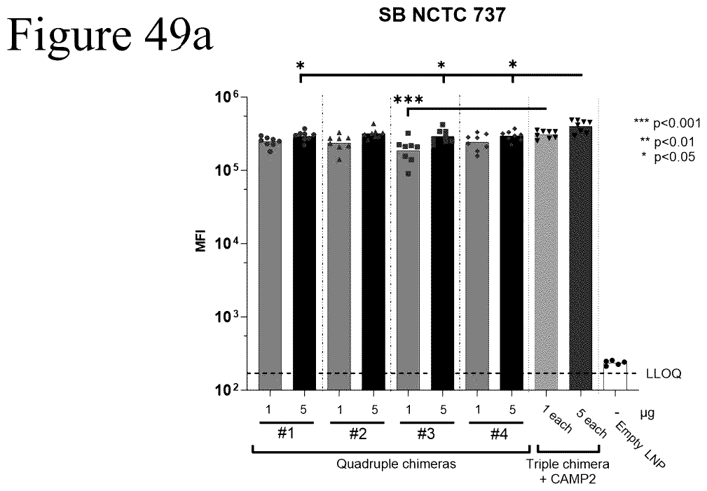

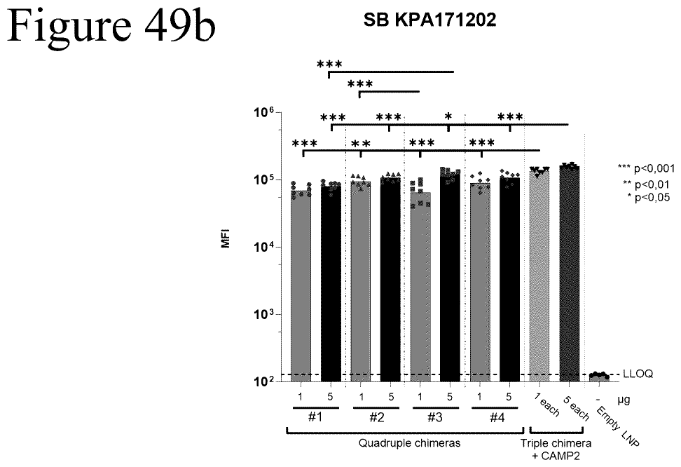

Both the “quadruple chimera” formulations and the “bivalent combination” formulation elicited SB titers, as measured with a Surface Binding (SB) assay and as shown in FIG. 49A-49B.

At the 1 μg dose, all quadruple chimeras (except quadruple chimera #3) induced similar SB value as the bivalent combination, in the SB assay on C. acnes strain NCTC737 (FIG. 49a). At the 5 μg dose, all four quadruple chimeras performed similarly. At this 5 μg dose, all quadruple chimeras (except quadruple chimera #2) induced lower SB values than the bivalent combination.

In the SB assay on C. acnes strain KPA171202 (FIG. 49b), at both the 1 μg and 5 μg doses, all quadruple chimeras induced lower SB value than the bivalent combination. The quadruple chimera #3 induced a higher SB value than the quadruple chimera #1 at 5 μg dose, and the quadruple chimera #2 induced a higher SB value than the quadruple chimera #3 at lug dose.

Chimeric C. acnes DsA1/DsA2/PITP/CAMP2 mRNA Constructs and a Bivalent Combination of a CAMP2-TMB mRNA Construct and a DsA1/DsA2/PITP mRNA Construct Elicit Antibodies which Neutralize the Co-Hemolytic Activity of CAMP 2

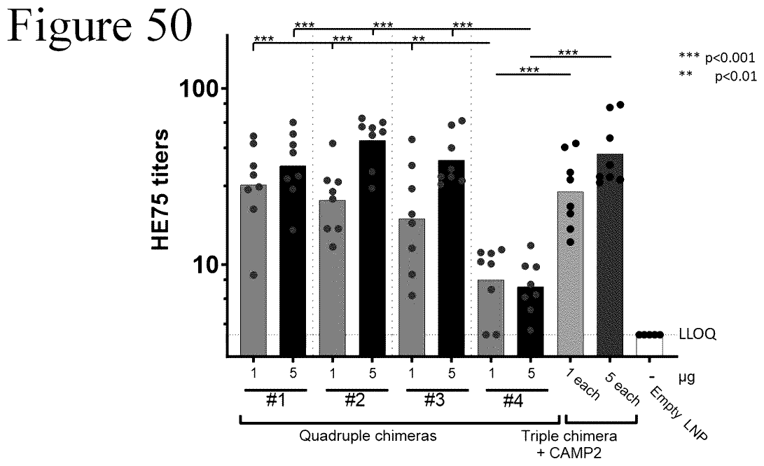

Both the “quadruple chimera” formulations and the “bivalent combination” formulation elicited antibodies which neutralize the co-hemolytic activity of CAMP2, as measured with a CAMP2 co-hemolytic neutralization assay (FIG. 50).

At equivalent dose, all quadruple chimeras (except quadruple chimera #4) and the bivalent combination elicited comparable co-hemolytic activity neutralization HE75 titers.

Correlations Between Antibody Responses Elicited by Chimeric C. acnes DsA1/DsA2/PITP/CAMP2 mRNA Constructs and a Bivalent Combination of a CAMP2-TMB mRNA Construct and a DsA1/DsA2/PITP mRNA Construct

Good and significant Pearson correlations (r values ranging from 0.8 to 0.9 and all p-values below 0.001) were demonstrated between ELISA anti-DsA1 and -DsA2 IgG titers, Surface Binding titers and Opsonophagocytic Killing titers performed on NCTC737 C. acnes strain.

Good and significant Pearson correlations (r values ranging from 0.69 to 0.77 and all p-values below 0.001) were also demonstrated between ELISA anti-PITP IgG titers, Surface Binding titers and Opsonophagocytic Killing titers on KPA171202 C. acnes strain.

Finally, a good and significant Pearson correlation (r value of 0.82 with a p-value below 0.001) was demonstrated between ELISA anti-CAMP2 IgG titers and the neutralization titer of CAMP2 co-hemolytic activity.

### CONCLUSION

Overall, all quadruple chimeras induced functional antibody responses, which were comparable to the ones induced by the bivalent combination for the higher dose of 5 μg, except for the quadruple chimera #4 comprising the C-ter portion of CAMP2, relatively to the CAMP2 response. The high correlation of results between all readouts consolidates the conclusions of this study, and quadruple chimeras appear to be of interest for use as acne antigens. Compared to the bivalent combination, the quadruple chimeras could offer advantages as they may help limiting reactogenicity (by allowing to use a lower amount of cationic lipid/LNP per dose) and may allow an easier, faster and less expensive manufacturing process.

### Example 14—Synthesis of IS-001 According to Scheme 3

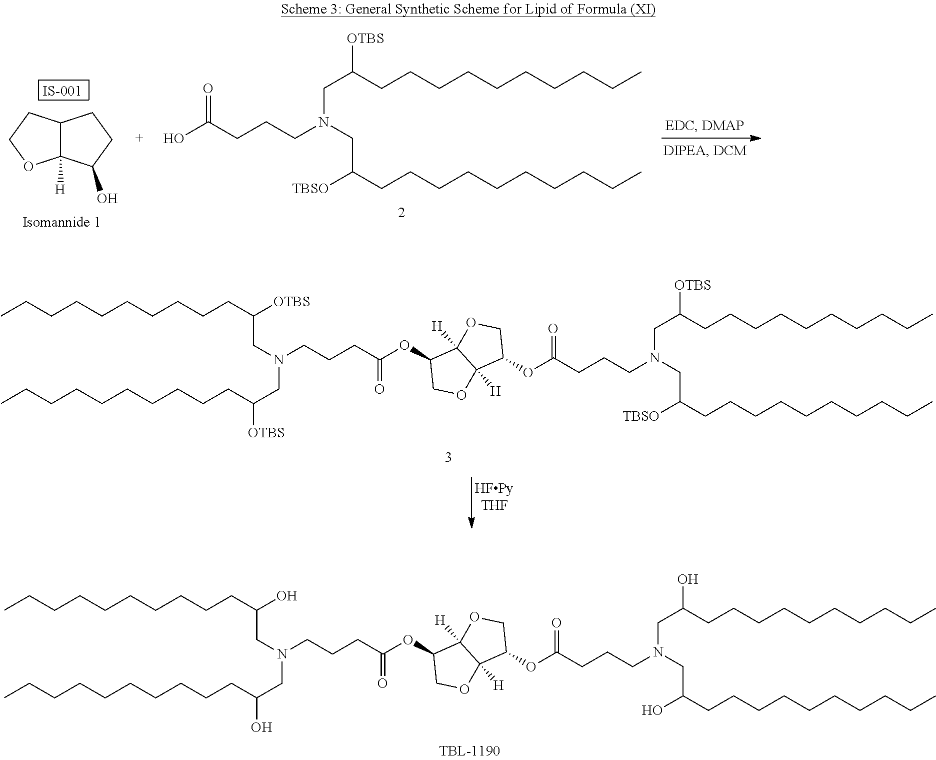

### Abbreviations:

DCM: Dichloromethane
DIPEA: N,N-Diisopropylethylamine
DMAP: 4-Dimethylaminopyridine
EDC: 1-Ethyl-3-(3-dimethylaminopropyl) carbodiimide
EtOAc: Ethyl acetate
NaHCO3: Sodium hydrogencarbonate
Py: Pyridine
Na₂SO4: Sodium Sulfate
TEA: Triethylamine
TFA: Trifluoroacetic Acid
MS: Mass spectrometry
ESI-MS: Electrospray ionization mass spectrometry
TLC: Thin Layer Chromatography

### Step 1: Synthesis of Intermediate (3)

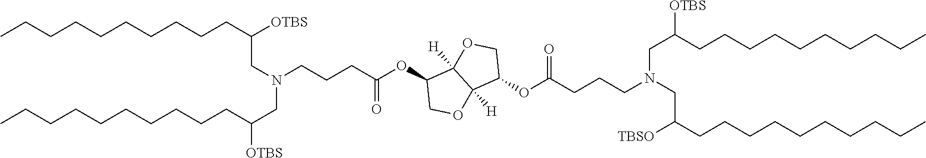 [SMILES: CCCCCCCCCCC(CN(CCCC(=O)O[C@H]1CO[C@H]2[C@@H]1OC[C@H]2OC(=O)CCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 3: To a solution of acid (2) (1.2 g, 1.71 mmol) and isosorbide (1) (0.100 g, 0.68 mmol) in dichloromethane (10 mL) were added DIPEA (0.95 mL, 5.47 mmol), DMAP (0.084 g, 0.68 mmol) and EDC (0.393 g, 2.05 mmol). The resulting mixture was stirred at room temperature for overnight. After 16 h, MS and TLC (30% EtOAc in hexanes) analysis indicated completion of the reaction. The reaction mixture was diluted with dichloromethane and washed with saturated NaHCO₃ solution, water and brine solution. The organic layer was dried over anhydrous Na₂SO4 and concentrated. The crude residue was purified, and the desired product was eluted at 6% EtOAc in hexanes. The product containing fractions were concentrated to obtain 0.72 g (69%) of pure product.

### Results:

ESI-MS: Calculated C₈₆H₁₇₇N₂O₁₀Si₄, [M+H⁺]=1510.25, Observed=1510.3 and 755.4 [M/2+H⁺]

### Step 2: Synthesis of IS-001

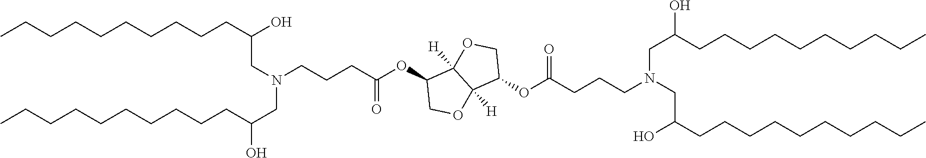 [SMILES: CCCCCCCCCCC(O)CN(CCCC(=O)O[C@H]1CO[C@H]2[C@@H]1OC[C@H]2OC(=O)CCCN(CC(O)CCCCCCCCCC)CC(O)CCCCCCCCCC)CC(O)CCCCCCCCCC]

As depicted in Scheme 3: To a solution of Intermediate (3) (0.72 g, 0.476 mmol) in tetrahydrofuran (4 mL) was added hydrogen fluoride (70% HF.py complex, 2 mL, 14.298 mmol) at 0° C. and stirred at the same temperature for 5 minutes. Then reaction mixture was warmed to room temperature and stirred for 16 h. MS analysis indicated completion of the reaction. The reaction mixture was diluted with ethyl acetate, quenched by slow addition of solid NaHCO3 at 0° C., followed by saturated NaHCO₃ solution. The organic layer was washed with sat. NaHCO₃ solution, water and brine. Then dried over anhydrous Na₂SO4 and concentrated. The crude residue was purified, and the desired product was eluted at 65% EtOAc in hexanes. The purest fractions were concentrated to obtain 0.120 g (24%) of pure product.

### Results:

¹H NMR (400 MHZ, CDCl₃) δ 5.30-5.00 (m, 2H), 4.97-4.68 (m, 2H), 4.55-3.71 (m, 8H), 3.57-2.92 (m, 8H), 2.84-2.04 (m, 8H), 1.99-1.01 (m, 76H), 0.88 (t, J=6.8 Hz, 12H).

ESI-MS: Calculated C₆₂H₁₂₁N₂O₁₀, [M+H⁺]=1053.90, Observed=1053.2 and 527.3 [M/2+H⁺]

### Example 15—Synthesis and Characterisation of Modified CAMP2 mRNA Constructs

Plasmids with a DNA sequence encoding a modified CAMP2 polypeptide (a CAMP2 sequence, a signal sequence (HA SS) and a transmembrane domain sequence (HA TMB) of Influenza Hemagglutinin (HA, H1N1 A/Caledonia/20/1999; SEQ ID NO: 84) at N-terminus and C-terminus respectively) operably linked to RNA polymerase promoter were linearized with a restriction enzyme and purified. mRNA transcripts were synthesized using modified nucleotides (in which all uridine nucleosides were replaced with 1-methyl pseudo uridine) by in vitro transcription using SP6 RNA Polymerase from the purified and linearized plasmid as described in US20230407358. The purified mRNA from the in vitro transcription step was capped and tailed as described in US20230407358.

Four mRNA constructs were generated from different plasmid sequences:
mRNA #1 comprising SEQ ID NO: 90
mRNA #2 comprising SEQ ID NO: 91
mRNA #3 comprising SEQ ID NO: 391
mRNA #4 comprising SEQ ID NO: 392.

Each mRNA also comprised a cap1, a 5′UTR from CMV
given by the sequence:
(SEQ ID NO: 265)
GGACAGAUCGCCUGGAGACGCCAUCCACGCUGUUUUGACCUCCAUAGAA
GACACCGGGACCGAUCCAGCCUCCGCGGCCGGGAACGGUGCAUUGGAAC
GCGGAUUCCCCGUGCCAAGAGUGACUCACCGUCCUUGACACG,
a 3' UTR from hGH given by the sequence:
(SEQ ID NO: 266)
CGGGUGGCAUCCCUGUGACCCCUCCCCAGUGCCUCUCCUGGCCCUGGAA
GUUGCCACUCCAGUGCCCACCAGCCUUGUCCUAAUAAAAUUAAGUUGCA
UC,

and a polyA tail.

With respect to mRNA size, tail size, % of tailed population and cap1%, all mRNAs met the targeted specifications (see Table 12). Tailing of the mRNA transcripts was assessed by capillary electrophoresis as described in US20230407358. The length of the mRNA tail was measured using capillary electrophoresis on a Fragment Analyzer instrument (Advanced Analytical). Capping of the mRNA transcripts was assessed by liquid chromatography mass spectrometry (LC/MS) as described in US20230407358.

TABLE 12
Characterisation of HA SS_CAMP2_WT_HA TMB mRNAs

| Construct sequence | Tail Length (nt) | % Tailing | Cap1 % |
|---|---|---|---|
| SEQ ID NO: 90 | 455 | 95.5% | 91% |
| SEQ ID NO: 91 | 430 | 97% | 97% |
| SEQ ID NO: 391 | 481 | 86% | 88% |
| SEQ ID NO: 392 | 423 | 95% | 95% |

mRNAs comprising the sequence according to SEQ ID NO: 90, 91 or 392 showed higher % of tailed/capped product. Higher % of tailed/capped product should result in better translation efficiency.

### Polypeptide Sequences

TABLE 5

| | | | | contains | | | Encoded, |
|---|---|---|---|---|---|---|---|
| | | | | a | | | for |
| | | | | secretion | contains | | example, |
| | | | | signal | a | contains | by the |
| | | | | peptide | C. acnes | a TMB | nucleotide |
| | | | | sequence | antigen | sequence | sequence |
| SEQ | | | | according | according | according | according |
| ID | | | Alternative | to SEQ | to SEQ ID | to SEQ | to SEQ |
| NO | protein sequence | name | name | ID NO | NO | ID NO | ID NO |
| 1 | | | rProt CAMP2 | | 43 | | 289 |
| 2 | | ssHA_CAMP2_ | mRNA/ssHA_CAMP2_ | 42 | 44 | | 87 |
| | | WT_GA | WT_GA | | | | |
| 3 | | ssHA_CAMP2_ | mRNA/ssHA_CAMP2_ | 42 | 45 | | 88 |
| | | WT_GS | WT_GS | | | | |
| 4 | | ssHA_CAMP2_ | mRNA/HA | 42 | 46 | | 89 |
| | | WT_G+ | SS_CAMP2_WT | | | | |
| 5 | | CAMP2_WT_HA | mRNA/HA | 42 | 47 | 84 | 90 |
| | | TMB | SS_CAMP2_WT_HA | | | | |
| | | | TMB #1 | | | | |
| 6 | MKAKLLVLLCTFTATYAVEPT | CAMP2_WT_HA | mRNA/HA | 42 | 48 | 84 | 91 |
| | TTISATSTHELSASDARNSIQ | TMB | SS_CAMP2_WT_HA | | | | |
| | LLNAHIATLQSVQKSVPGSDY | | TMB #2 | | | | |
| | SDQIRDLLKAAFDLRGLIETL | | | | | | |
| | AHGGIPFYDPSTIMPRIKLVA | | | | | | |
| | TTIDTIHTATTTLQNKVRPAH | | | | | | |
| | VELGLEVTKAVLLTANPASTA | | | | | | |
| | KELDAEGAALKARLEKVSQYP | | | | | | |
| | DLTPNDVATVYVRTNFSKTIW | | | | | | |
| | QVRANRDRYILGHKSAAVYKT | | | | | | |
| | LNHAITKAVGVRLNPKTTVGN | | | | | | |
| | IQAARTELLAAYQTAFNSPDV | | | | | | |
| | KKAAsgsILAIYSTVASSLVL | | | | | | |
| | LVSLGAISFgsg | | | | | | |
| 7 | | | mRNA/NA | | 49 | | 92 |
| | | | TMB_CAMP2_WT | | | | |
| 8 | | | mRNA/NA | | 50 | | 93 |
| | | | TMB_CAMP2_Nglyc- | | | | |
| | | | neg | | | | |
| 9 | | CAMP2_G-_HA | mRNA/HA | 42 | 51 | 84 | 94 |
| | | TMB | SS_CAMP2_Nglyc- | | | | |
| | | | neg_HA TMB | | | | |
| 10 | | | mRNA/ssHA_ | 42 | 52 | | 95 |
| | | | CAMP2_G-GA | | | | |
| 11 | | | mRNA/ssHA_CAMP2_ | 42 | 53 | | 96 |
| | | | G-GS | | | | |
| 12 | | ssHA_CAMP2_ | mRNA/HA | 42 | 54 | | 97 |
| | | WT_G- | SS_CAMP2_ | | | | |
| | | | Nglyc-neg | | | | |
| 13 | | ssHA_CAMP2_ | mRNA/HA | 42 | 55 | | 98 |
| | | mL | SS_CAMP2_mL | | | | |
| 14 | | ssHA_CAMP2_ | mRNA/HA | 42 | 56 | | 99 |
| | | G(-)_mL | SS_CAMP2_Nglyc- | | | | |
| | | | neg_mL | | | | |
| 15 | | ssHA_CAMP2 | mRNA/HA | 42 | 57 | | 100 |
| | | mCter | SS_CAMP2_mCter | | | | |
| 16 | | ssHA_CAMP2_ | mRNA/HA SS_CAMP2_ | 42 | 58 | | 101 |
| | | G-_mCter | Nglyc-neg_mCter | | | | |
| 17 | | ssHA_DSA1_ | mRNA/HA SS_DSA1_ | 42 | 59 | 85 | 102 |
| | | Native_TMB_ | HA TMB | | | | |
| | | WT_trunc | | | | | |
| 18 | | ssHA_DSA1_ | mRNA/HA | 42 | 60 | | 103 |
| | | WT_trunc | SS_DSA1_WT trunc | | | | |
| 19 | | ssHA_DSA1_ | mRNA/HA | 42 | 61 | 84 | 104 |
| | | SGS_TMB_WT_ | SS_DSA1_ | | | | |
| | | trunc | HA TMB-sgs | | | | |
| | | | linker | | | | |
| 20 | | ssHA_DSA2_ | mRNA/HA | 42 | 62 | 85 | 105 |
| | | Native_TMB_ | SS_DSA2_HA | | | | |
| | | WT_trunc | TMB | | | | |
| 21 | | ssHA_DSA2_ | mRNA/HA | 42 | 63 | 84 | 106 |
| | | SGS_TMB_WT_ | SS_DSA2_HA | | | | |
| | | trunc | TMB-sgs | | | | |
| | | | linker | | | | |
| 22 | | ssHA_DSA2_ | mRNA/HA | 42 | 64 | | 107 |
| | | WT_trunc | SS_DSA2_WT | | | | |
| | | | trunc | | | | |
| 23 | | ssHA_DSA2_ | mRNA/HA | 42 | 65 | | 108 |
| | | WT_trunc_ | SS_DSA2_ | | | | |
| | | mutH | mutH | | | | |
| 24 | | ssHA_DSA2_ | mRNA/HA | 42 | 66 | 85 | 109 |
| | | Native_TMB_ | SS_DSA2- | | | | |
| | | WT_trunc_ | mutH_HA | | | | |
| | | mut | TMB | | | | |
| 25 | | ssHA_DSA2_ | mRNA/HA | 42 | 67 | 84 | 110 |
| | | SGS_TMB_WT_ | SS_DSA2- | | | | |
| | | trunc_mut | mutH_HA | | | | |
| | | | TMB-sgs linker | | | | |
| 26 | | | mRNA/HA | 42 | 68 | 84 | 111 |
| | | | SS_DSA2-Nglyc- | | | | |
| | | | neg_HA | | | | |
| | | | TMB-sgs linker | | | | |
| 27 | | | mRNA/HA | 42 | 69 | 86 | 112 |
| | | | SS_DSA2- | | | | |
| | | | Nglyc-neg_HA | | | | |
| | | | TMB | | | | |
| 28 | MKAKLLVLLCTFTATYASSNR | ssHA_H4_V3 | mRNA/ssHA_H4- | 42 | 70 | | 113 |
| | PRSVAQAAIATDGKGIIDKDS | | V3 | | | | |
| | RDAVINDAKLRAAIAGALVKA | | | | | | |
| | GFSSADAVALAPRIAKEMAKE | | | | | | |
| | GVLLINHHKLKALIGAQLGLL | | | | | | |
| | TDAKIQRAAAAVDLGIKATLA | | | | | | |
| | ATIIPNALGSAAFKNAVIANL | | | | | | |
| | VAAGIDKHLARATAVAIVATA | | | | | | |
| | LNPALGPIAKFELIKAEIAAQ | | | | | | |
| | AALLIRRGVHLQKAAIEHVIG | | | | | | |
| | RAFDAAVATAIISSPILSARI | | | | | | |
| | VTHLVRAGIDKSIAISLAPHI | | | | | | |
| | VKRLAKEPLLAFNTAKLMKNI | | | | | | |
| | TRQIVDVITADKAIKTAEQLE | | | | | | |
| | KELPALDDLVKKASSPPKPTP | | | | | | |
| | TPTPTPT | | | | | | |
| 29 | | | rProt H4-V3 | | 71 | | |
| 30 | | | mRNA/ssHA_H4- | 42 | 72 | | 114 |
| | | | V3 Nglyc-neg | | | | |
| 31 | MKAKLLVLLCTFTATYAAGPT | ssHA_P028_ | mRNA/P28-V7 | 42 | 73 | | 115 |
| | VTVIPVGREGGDITISGKGFS | V7 | | | | | |
| | TTGFGVYVAVAPASVPEFYGN | | | | | | |
| | SDKFYGYDPSKDTTESPSTIW | | | | | | |
| | VYTPSQKAIGSRFAQGRPMNN | | | | | | |
| | DGSFTITMKAPPFEQGKDFVV | | | | | | |
| | LTTKAHGVGKTDHSDDTRTPV | | | | | | |
| | TYREATPAPTGPKTPIAPSKQ | | | | | | |
| | PSKQAAPSKQVKPSKQAGPNK | | | | | | |
| | QSTTPQQKTAEHRSQTPAAHR | | | | | | |
| | TMTKQVSTIGASKVTSGSLTW | | | | | | |
| | GIRTSFTSYLRGPIANGSWKL | | | | | | |
| | SGGANWNGSAFTFPLTSGSFD | | | | | | |
| | PATKSGSLKYSGSVHMTGHHG | | | | | | |
| | ILDMTLAEPSLQIKGSTGHLY | | | | | | |
| | LDVKSSSMDGKKTNYGRVDFA | | | | | | |
| | TFGVSVSGNAAIKGSPVKLTA | | | | | | |
| | TGAKAFAGFYRAGEPMNPLST | | | | | | |
| | NLTLSAEKVSHNVTVDAVTGK | | | | | | |
| | VIGDDSGKGAGRGLPVT | | | | | | |
| 32 | | | rProt P28-V7 | | 74 | | |
| 33 | | ssHA_PITP_ | mRNA/ssHA_PITP_ | 42 | 75 | | 117 |
| | | WT_trunc | WT_trunc | | | | |
| 34 | | | mRNA/ssHA_PITP_ | 42 | 76 | | 118 |
| | | | Nglyc-neg | | | | |
| 35 | | P028_V7_ | mRNA/ssHA_P28-V7_ | 42 | 77 | | 119 |
| | | Glyneg | Nglyc-neg | | | | |
| 36 | | ssHA_PITP_ | mRNA/ssHA_PITP_ | 42 | 78 | 84 | 120 |
| | | SGS_TMB_HA_ | WT_HA TMB-sgs | | | | |
| | | WT_trunc | linker | | | | |
| 37 | | | mRNA/ssHA_PITP_ | 42 | 79 | 84 | 121 |
| | | | Nglyc-neg_HA TMB- | | | | |
| | | | sgs linker | | | | |
| 38 | MKAKLLVLLCTFTATYASSNR | nCO- | H4-V3-f-PRO-028- | 42 | 80 | | 122 |
| | PRSVAQAAIATDGKGIIDKDS | ssHA_H4- | V7-F16, triple | | | | |
| | RDAVINDAKLRAAIAGALVKA | V3-f-PRO- | chimera | | | | |
| | GFSSADAVALAPRIAKEMAKE | 028-V7-F16 | P22/P27/P28 | | | | |
| | GVLLINHHKLKALIGAQLGLL | | | | | | |
| | TDAKIQRAAAAVDLGIKATLA | | | | | | |
| | ATIIPNALGSAAFKNAVIANL | | | | | | |
| | VAAGIDKHLARATAVAIVATA | | | | | | |
| | LNPALGPIAKFELIKAEIAAQ | | | | | | |
| | AALLIRRGVHLQKAAIEHVIG | | | | | | |
| | RAFDAAVATAIISSPILSARI | | | | | | |
| | VTHLVRAGIDKSIAISLAPHI | | | | | | |
| | VKRLAKEPLLAFNTAKLMKNI | | | | | | |
| | TRQIVDVITADKAIKTAEQLE | | | | | | |
| | KELPALDDLVKKASSPPKPTP | | | | | | |
| | TPTPTPTAGPTVTVIPVGREG | | | | | | |
| | GDITISGKGFSTTGFGVYVAV | | | | | | |
| | APASVPEFYGNSDKFYGYDPS | | | | | | |
| | KDTTESPSTIWVYTPSQKAIG | | | | | | |
| | SRFAQGRPMNNDGSFTITMKA | | | | | | |
| | PPFEQGKDFVVLTTKAHGVGK | | | | | | |
| | TDHSDDTRTPVTYREATPAPT | | | | | | |
| | GPKTPI | | | | | | |
| 39 | | nCO- | H4-V3 low glyc | 42 | 81 | | 123 |
| | | ssHA_PRO- | | | | | |
| | | H4-V3- | | | | | |
| | | lowglyc | | | | | |
| 40 | MKAKLLVLLCTFTATYASSNR | gc.CO- | PRO-028-V7-C12 | 42 | 82 | | 124 |
| | PRSVAQAAIATDGKGIIDKDS | ssHA_PRO- | lowglyc | | | | |
| | RDAVINDAKLRAAIAGALVKA | 028-V7-C12- | | | | | |
| | GFSSADAVALAPRIAKEMAKE | lowglyc | | | | | |
| | GVLLINHHKLKALIGAQLGLL | | | | | | |
| | TDAKIQRAAAAVDLGIKATLA | | | | | | |
| | ATIIPNALGSAAFKNAVIANL | | | | | | |
| | VAAGIDKHLARATAVAIVATA | | | | | | |
| | LNPALGPIAKFELIKAEIAAQ | | | | | | |
| | AALLIRRGVHLQKAAIEHVIG | | | | | | |
| | RAFDAAVATAIISSPILSARI | | | | | | |
| | VTHLVRAGIDKSIAISLAPHI | | | | | | |
| | VKRLAKEPLLAFNTAKLMKNI | | | | | | |
| | TRQIVDVITADKAIKTAEQLE | | | | | | |
| | KELPALDDLVKKAMGPPKPTG | | | | | | |
| | GGGGAGPTVTVIPVGREGGDI | | | | | | |
| | TISGKGFSTTGFGVYVAVAPA | | | | | | |
| | SVPEFYGNSDKFYGYDPSKDT | | | | | | |
| | TESPSTIWVYTPSQKAIGSRF | | | | | | |
| | AQGRPMNNDGSFTITMKAPPF | | | | | | |
| | EQGKDFVVLTTKAHGVGKGDH | | | | | | |
| | GDDTRTPVGYR | | | | | | |
| 41 | MKAKLLVLLCTFTATYASSNR | nCO- | PRO-028-V7-C12, | 42 | 83 | | 125 |
| | PRSVAQAAIATDGKGIIDKDS | ssHA_PRO- | triple chimera | | | | |
| | RDAVINDAKLRAAIAGALVKA | 028-V7-C12 | P22/P27/P28 | | | | |
| | GFSSADAVALAPRIAKEMAKE | | | | | | |
| | GVLLINHHKLKALIGAQLGLL | | | | | | |
| | TDAKIQRAAAAVDLGIKATLA | | | | | | |
| | ATIIPNALGSAAFKNAVIANL | | | | | | |
| | VAAGIDKHLARATAVAIVATA | | | | | | |
| | LNPALGPIAKFELIKAEIAAQ | | | | | | |
| | AALLIRRGVHLQKAAIEHVIG | | | | | | |
| | RAFDAAVATAIISSPILSARI | | | | | | |
| | VTHLVRAGIDKSIAISLAPHI | | | | | | |
| | VKRLAKEPLLAFNTAKLMKNI | | | | | | |
| | TRQIVDVITADKAIKTAEQLE | | | | | | |
| | KELPALDDLVKKASSPPKPTP | | | | | | |
| | TPTPTPTAGPTVTVIPVGREG | | | | | | |
| | GDITISGKGFSTTGFGVYVAV | | | | | | |
| | APASVPEFYGNSDKFYGYDPS | | | | | | |
| | KDTTESPSTIWVYTPSQKAIG | | | | | | |
| | SRFAQGRPMNNDGSFTITMKA | | | | | | |
| | PPFEQGKDFVVLTTKAHGVGK | | | | | | |
| | TDHSDDTRTPVTYR | | | | | | |
| 313 | | C. acnes | | | 339 | | |
| | | CAMP2 | | | | | |
| | | from strain | | | | | |
| | | ATCC6919 | | | | | |

| SEQ ID | contains a C. acnes antigen |
|---|---|
| NO | according to SEQ ID NO |
| 314 | 339 |
| 315 | 340 |
| 316 | 341 |
| 317 | 342 |
| 318 | 343 |
| 319 | 344 |
| 320 | 345 |
| 321 | 346 |
| 322 | 347 |
| 323 | 348 |
| 324 | 349 |
| 325 | 350 |
| 326 | 351 |
| 327 | 352 |
| 328 | 353 |
| 329 | 354 |
| 330 | 355 |
| 331 | 356 |
| 332 | 357 |
| 333 | 358 |
| 334 | 359 |
| 335 | 360 |
| 336 | 361 |
| 337 | 362 |
| 338 | 363 |

| | | | | contains | | | Encoded, |
|---|---|---|---|---|---|---|---|
| | | | | a | | | for |
| | | | | secretion | contains | | example, |
| | | | | signal | a | contains | by the |
| | | | | peptide | C. acnes | a TMB | nucleotide |
| | | | | sequence | antigen | sequence | sequence |
| SEQ | | | | according | according | according | according |
| ID | | | Alternative | to SEQ | to SEQ | to SEQ | to SEQ |
| NO | Protein sequence | name | name | ID NO | ID NO | ID NO | ID NO |
| 367 | | P028- | | 42 | 368 | | |
| | | V7-C12- | | | | | |
| | | lowglyc-V2 | | | | | |
| 369 | MKAKLLVLLCTFTATYAVEPT | Quad-CAMP2 | | | 373 | | 377, 378 |
| | TTISATSTHELSASDARNSIQ | H4V3-f- | | | | | |
| | LLNAHIATLQSVQKSVPGSDY | PRO-028-V7 | | | | | |
| | SDQIRDLLKAAFDLRGLIETL | F16_sgs_HA- | | | | | |
| | AHGGIPFYDPSTIMPRIKLVA | TM_gsg_ssHA | | | | | |
| | TTIDTIHTATTITTLQNKVRP | | | | | | |
| | AHVELGLEVTKAVLLTANPAS | | | | | | |
| | TAKELDAEGAALKARLEKVSQ | | | | | | |
| | YPDLTPNDVATVYVRTNFSKT | | | | | | |
| | IWQVRANRDRYILGHKSAAVY | | | | | | |
| | KTLNHAITKAVGVRLNPKTTV | | | | | | |
| | GNIQAARTELLAAYQTAFNSP | | | | | | |
| | DVEKKAASSNRPRSVAQAAIA | | | | | | |
| | TDGKGIIDKDSRDAVINDAKL | | | | | | |
| | RAAIAGALVKAGFSSADAVAL | | | | | | |
| | APRIAKEMAKEGVLLINHHKL | | | | | | |
| | KALIGAQLGLLTDAKIQRAAA | | | | | | |
| | AVDLGIKATLAATIPNALGSA | | | | | | |
| | AFKNAVIANLVAAGIDKHLAR | | | | | | |
| | ATAVAIVATALNPALGPIAKF | | | | | | |
| | ELIKAEIAAQAALLIRRGVHL | | | | | | |
| | QKAAIEHVIGRAFDAAVATAI | | | | | | |
| | ISSPILSARIVTHLVRAGIDK | | | | | | |
| | SIAISLAPHIVKRLAKEPLLA | | | | | | |
| | FNTAKLMKNITRQIVDVITAD | | | | | | |
| | KAIKTAEQLEKELPALDDLVK | | | | | | |
| | KASSPPKPTPTPTPTPTAGPT | | | | | | |
| | VTVIPVGREGGDITISGKGFS | | | | | | |
| | TTGFGVYVAVAPASVPEFYGN | | | | | | |
| | SDKFYGYDPSKDTTESPSTIW | | | | | | |
| | VYTPSQKAIGSRFAQGRPMNN | | | | | | |
| | DGSFTITMKAPPFEQGKDFVV | | | | | | |
| | LTTKAHGVGKTDHSDDTRTPV | | | | | | |
| | TYREATPAPTGPKTPISGSIL | | | | | | |
| | AIYSTVASSLVLLVSLGAISF | | | | | | |
| | GSG | | | | | | |
| 370 | MKAKLLVLLCTFTATYAVEPT | Quad-CAMP2- | | | 374 | | 380 |
| | TTISATSTHELSASDARNSIQ | H4V3-f-PRO- | | | | | |
| | LLNAHIATLQSVQKSVPGSDY | 028-V7- | | | | | |
| | SDQIRDLLKAAFDLRGLIETL | F16_ssHA | | | | | |
| | AHGGIPFYDPSTIMPRIKLVA | | | | | | |
| | TTIDTIHTATTTLQNKVRPAH | | | | | | |
| | VELGLEVTKAVLLTANPASTA | | | | | | |
| | KELDAEGAALKARLEKVSQYP | | | | | | |
| | DLTPNDVATVYVRTNFSKTIW | | | | | | |
| | QVRANRDRYILGHKSAAVYKT | | | | | | |
| | LNHAITKAVGVRLNPKTTVGN | | | | | | |
| | IQAARTELLAAYQTAFNSPDV | | | | | | |
| | KKAASSNRPRSVAQAAIATDG | | | | | | |
| | KGIIDKDSRDAVINDAKLRAA | | | | | | |
| | IAGALVKAGFSSADAVALAPR | | | | | | |
| | IAKEMAKEGVLLINHHKLKAL | | | | | | |
| | IGAQLGLLTDAKIQRAAAAVD | | | | | | |
| | LGIKATLAATIIPNALGSAAF | | | | | | |
| | KNAVIANLVAAGIDKHLARAT | | | | | | |
| | AVAIVATALNPALGPIAKFEL | | | | | | |
| | IKAEIAAQAALLIRRGVHLQK | | | | | | |
| | AAIEHVIGRAFDAAVATAIIS | | | | | | |
| | SPILSARIVTHLVRAGIDKSI | | | | | | |
| | AISLAPHIVKRLAKEPLLAFN | | | | | | |
| | TAKLMKNITRQIVDVITADKA | | | | | | |
| | IKTAEQLEKELPALDDLVKKA | | | | | | |
| | SSPPKPTPTPTPTPTAGPTVT | | | | | | |
| | VIPVGREGGDITISGKGFSTT | | | | | | |
| | GFGVYVAVAPASVPEFYGNSD | | | | | | |
| | KFYGYDPSKDTTESPSTIWVY | | | | | | |
| | TPSQKAIGSRFAQGRPMNNDG | | | | | | |
| | SFTITMKAPPFEQGKDFVVLT | | | | | | |
| | TKAHGVGKTDHSDDTRTPVTY | | | | | | |
| | REATPAPTGPKTPI | | | | | | |
| 371 | MKAKLLVLLCTFTATYAVEPT | Quad-CAMP2- | | | 375 | | 381, 382 |
| | TTISATSTHELSASDARNSIQ | Nter-link- | | | | | |
| | LLNAHIATLQSVQKSVPGSDY | H4V3-f-PRO- | | | | | |
| | SDQIRDLLKAAFDLRGLIETL | 028-V7- | | | | | |
| | AHGGIPFYDPSTIMPRIKLVA | F16_ssHA | | | | | |
| | TTIDTIHTATTTLQNKVRPAH | | | | | | |
| | VELGLEVTKAVLLTANPASTA | | | | | | |
| | KELDAEGAALKARLEKVSQYP | | | | | | |
| | DLTPNDVATSSNRPRSVAQAA | | | | | | |
| | IATDGKGIIDKDSRDAVINDA | | | | | | |
| | KLRAAIAGALVKAGFSSADAV | | | | | | |
| | ALAPRIAKEMAKEGVLLINHH | | | | | | |
| | KLKALIGAQLGLLTDAKIQRA | | | | | | |
| | AAAVDLGIKATLAATIIPNAL | | | | | | |
| | GSAAFKNAVIANLVAAGIDKH | | | | | | |
| | LARATAVAIVATALNPALGPI | | | | | | |
| | AKFELIKAEIAAQAALLIRRG | | | | | | |
| | VHLQKAAIEHVIGRAFDAAVA | | | | | | |
| | TAIISSPILSARIVTHLVRAG | | | | | | |
| | IDKSIAISLAPHIVKRLAKEP | | | | | | |
| | LLAFNTAKLMKNITRQIVDVI | | | | | | |
| | TADKAIKTAEQLEKELPALDD | | | | | | |
| | LVKKASSPPKPTPTPTPTPTA | | | | | | |
| | GPTVTVIPVGREGGDITISGK | | | | | | |
| | GFSTTGFGVYVAVAPASVPEF | | | | | | |
| | YGNSDKFYGYDPSKDTTESPS | | | | | | |
| | TIWVYTPSQKAIGSRFAQGRP | | | | | | |
| | MNNDGSFTITMKAPPFEQGKD | | | | | | |
| | FVVLTTKAHGVGKTDHSDDTR | | | | | | |
| | TPVTYREATPAPTGPKTPI | | | | | | |
| 372 | MKAKLLVLLCTFTATYASSNR | Quad-H4V3- | | | 376 | | 383, 394 |
| | PRSVAQAAIATDGKGIIDKDS | f-PRO-028- | | | | | |
| | RDAVINDAKLRAAIAGALVKA | V7-F16- | | | | | |
| | GFSSADAVALAPRIAKEMAKE | nolink- | | | | | |
| | GVLLINHHKLKALIGAQLGLL | Cter- | | | | | |
| | TDAKIQRAAAAVDLGIKATLA | CAMP2_ssHA | | | | | |
| | ATIIPNALGSAAFKNAVIANL | | | | | | |
| | VAAGIDKHLARATAVAIVATA | | | | | | |
| | LNPALGPIAKFELIKAEIAAQ | | | | | | |
| | AALLIRRGVHLQKAAIEHVIG | | | | | | |
| | RAFDAAVATAIISSPILSARI | | | | | | |
| | VTHLVRAGIDKSIAISLAPHI | | | | | | |
| | VKRLAKEPLLAFNTAKLMKNI | | | | | | |
| | TRQIVDVITADKAIKTAEQLE | | | | | | |
| | KELPALDDLVKKASSPPKPTP | | | | | | |
| | TPTPTPTAGPTVTVIPVGREG | | | | | | |
| | GDITISGKGFSTTGFGVYVAV | | | | | | |
| | APASVPEFYGNSDKFYGYDPS | | | | | | |
| | KDTTESPSTIWVYTPSQKAIG | | | | | | |
| | SRFAQGRPMNNDGSFTITMKA | | | | | | |
| | PPFEQGKDFVVLTTKAHGVGK | | | | | | |
| | TDHSDDTRTPVTYREATPAPT | | | | | | |
| | GPKTPIVYVRTNFSKTIWQVR | | | | | | |
| | ANRDRYILGHKSAAVYKTLNH | | | | | | |
| | AITKAVGVRLNPKTTVGNIQA | | | | | | |
| | ARTELLAAYQTAFNSPDVKKA | | | | | | |
| | A | | | | | | |

TABLE 6

| | Encoded, for example, by the |
|---|---|
| SEQ ID | nucleotide sequence according |
| NO | to SEQ ID NO |
| 42 | any one of 126-152, 311 |
| 44 | 153 |
| 45 | 154 |
| 46 | 155 |
| 47 | 156 |
| 48 | 157 |
| 49 | 158 |
| 50 | 159 |
| 51 | 160 |
| 52 | 161 |
| 53 | 162 |
| 54 | 163 |
| 55 | 164 |
| 56 | 165 |
| 57 | 166 |
| 58 | 167 |
| 59 | 168 |
| 60 | 169 |
| 61 | 170 |
| 62 | 171 |
| 63 | 172 |
| 64 | 173 |
| 65 | 174 |
| 66 | 175 |
| 67 | 176 |
| 68 | 177 |
| 69 | 178 |

| | | Encoded, |
|---|---|---|
| | | for |
| | | example, |
| | | by the |
| | | nucleotide |
| | | sequence |
| SEQ | | according |
| ID | | to SEQ |
| NO | Amino acid sequence | ID NO |
| 70 | SSNRPRSVAQAAIATDGKGIIDKDSRDAVINDAKLRAAIAGALVKAGFSSADAVALAPRIAKEMAKEGVLLINHHKLK | 179 |
| | ALIGAQLGLLTDAKIQRAAAAVDLGIKATLAATIIPNALGSAAFKNAVIANLVAAGIDKHLARATAVAIVATALNPAL | |
| | GPIAKFELIKAEIAAQAALLIRRGVHLQKAAIEHVIGRAFDAAVATAIISSPILSARIVTHLVRAGIDKSIAISLAPH | |
| | IVKRLAKEPLLAFNTAKLMKNITRQIVDVITADKAIKTAEQLEKELPALDDLVKKASSPPKPTPTPTPTPT | |
| 71 | MSSNRPRSVAQAAIATDGKGIIDKDSRDAVINDAKLRAAIAGALVKAGFSSADAVALAPRIAKEMAKEGVLLINHHKL | 180 |
| | KALIGAQLGLLTDAKIQRAAAAVDLGIKATLAATIIPNALGSAAFKNAVIANLVAAGIDKHLARATAVAIVATALNPA | |
| | LGPIAKFELIKAEIAAQAALLIRRGVHLQKAAIEHVIGRAFDAAVATAIISSPILSARIVTHLVRAGIDKSIAISLAP | |
| | HIVKRLAKEPLLAFNTAKLMKNITRQIVDVITADKAIKTAEQLEKELPALDDLVKKASSPPKPTPTPTPTPT | |
| 72 | SSNRPRSVAQAAIATDGKGIIDKDSRDAVINDAKLRAAIAGALVKAGFSSADAVALAPRIAKEMAKEGVLLINHHKLK | 181 |
| | ALIGAQLGLLTDAKIQRAAAAVDLGIKATLAATIIPNALGSAAFKNAVIANLVAAGIDKHLARATAVAIVATALNPAL | |
| | GPIAKFELIKAEIAAQAALLIRRGVHLQKAAIEHVIGRAFDAAVATAIISSPILSARIVTHLVRAGIDKSIAISLAPH | |
| | IVKRLAKEPLLAFNTAKLMKQITRQIVDVITADKAIKTAEQLEKELPALDDLVKKASSPPKPTPTPTPTPT | |
| 73 | AGPTVTVIPVGREGGDITISGKGFSTTGFGVYVAVAPASVPEFYGNSDKFYGYDPSKDTTESPSTIWVYTPSQKAIGS | 182 |
| | RFAQGRPMNNDGSFTITMKAPPFEQGKDFVVLTTKAHGVGKTDHSDDTRTPVTYREATPAPTGPKTPIAPSKQPSKQA | |
| | APSKQVKPSKQAGPNKQSTTPQQKTAEHRSQTPAAHRTMTKQVSTIGASKVTSGSLTWGIRTSFTSYLRGPIANGSWK | |
| | LSGGANWNGSAFTFPLTSGSFDPATKSGSLKYSGSVHMTGHHGILDMTLAEPSLQIKGSTGHLYLDVKSSSMDGKKTN | |
| | YGRVDFATFGVSVSGNAAIKGSPVKLTATGAKAFAGFYRAGEPMNPLSTNLTLSAEKVSHNVTVDAVTGKVIGDDSGK | |
| | GAGRGLPVT | |
| 74 | | 183 |
| 75 | | 184 |
| 76 | | 185 |
| 77 | | 186 |
| 78 | | 187 |
| 79 | | 188 |
| 80 | SSNRPRSVAQAAIATDGKGIIDKDSRDAVINDAKLRAAIAGALVKAGFSSADAVALAPRIAKEMAKEGVLLINHHKLK | 189 |
| | ALIGAQLGLLTDAKIQRAAAAVDLGIKATLAATIIPNALGSAAFKNAVIANLVAAGIDKHLARATAVAIVATALNPAL | |
| | AKEPLLAFNTAKLMKNITRQIVDVITADKAIKTAEQLEKELPALDDLVKKASSPPKPTPTPTPTPTAGPTVTVIPVGR | |
| | EGGDITISGKGFSTTGFGVYVAVAPASVPEFYGNSDKFYGYDPSKDTTESPSTIWVYTPSQKAIGSRFAQGRPMNNDG | |
| | SFTITMKAPPFEQGKDFVVLTTKAHGVGKTDHSDDTRTPVTYREATPAPTGPKTPI | |
| 81 | | 190 |
| 82 | SSNRPRSVAQAAIATDGKGIIDKDSRDAVINDAKLRAAIAGALVKAGFSSADAVALAPRIAKEMAKEGVLLINHHKLK | 191 |
| | ALIGAQLGLLTDAKIQRAAAAVDLGIKATLAATIIPNALGSAAFKNAVIANLVAAGIDKHLARATAVAIVATALNPAL | |
| | GPIAKFELIKAEIAAQAALLIRRGVHLQKAAIEHVIGRAFDAAVATAIISSPILSARIVTHLVRAGIDKSIAISLAPH | |
| | IVKRLAKEPLLAFNTAKLMKNITRQIVDVITADKAIKTAEQLEKELPALDDLVKKAMGPPKPTGGGGGAGPTVTVIPV | |
| | GREGGDITISGKGFSTTGFGVYVAVAPASVPEFYGNSDKFYGYDPSKDTTESPSTIWVYTPSQKAIGSRFAQGRPMNN | |
| | DGSFTITMKAPPFEQGKDFVVLTTKAHGVGKGDHGDDTRTPVGYR | |
| 83 | SSNRPRSVAQAAIATDGKGIIDKDSRDAVINDAKLRAAIAGALVKAGFSSADAVALAPRIAKEMAKEGVLLINHHKLK | 192 |
| | ALIGAQLGLLTDAKIQRAAAAVDLGIKATLAATIIPNALGSAAFKNAVIANLVAAGIDKHLARATAVAIVATALNPAL | |
| | GPIAKFELIKAEIAAQAALLIRRGVHLQKAAIEHVIGRAFDAAVATAIISSPILSARIVTHLVRAGIDKSIAISLAPH | |
| | IVKRLAKEPLLAFNTAKLMKNITRQIVDVITADKAIKTAEQLEKELPALDDLVKKASSPPKPTPTPTPTPTAGPTVTV | |
| | IPVGREGGDITISGKGFSTTGFGVYVAVAPASVPEFYGNSDKFYGYDPSKDTTESPSTIWVYTPSQKAIGSRFAQGRP | |
| | MNNDGSFTITMKAPPFEQGKDFVVLTTKAHGVGKTDHSDDTRTPVTYR | |
| 84 | sgsILAIYSTVASSLVLLVSLGAISFgsg | 193, 194, |
| | | 195, 197, |
| | | 198, 200, |
| | | 201, 395 |
| | | or 396 |
| 85 | | 196 or |
| | | 199 or |
| | | 312 |
| 86 | | 196 or |
| | | 199 or |
| | | 312 |
| 203 | VEPTTTISATSTHELSASDARNSIQLLNAHIATLQSVQKSVPGSDYSDQIRDLLKAAFDLRGLIETLAHGGIPFYDPS | |
| | TIMPRIKLVATTIDTIHTATTTLQNKVRPAHVELGLEVTKAVLLTANPASTAKELDAEGAALKARLEKVSQYPDLTPN | |
| | DVATVYVRTNFSKTIWQVRANRDRYILGHKSAAVYKTLNHAITKAVGVRLNPKTTVGNIQAARTELLAAYQTAFNSPD | |
| | VKKAA | |
| 207 | VEPTTTISATSTHELSASDARNSIQLLNAHIATLQSVQKSVPGSDYSDQIRDLLKAAFDLRGLIETLAHGGIPFYDPS | |
| | TIMPRIKLVATTIDTIHTATTTLQNKVRPAHVELGLEVTKAVLLTANPASTAKELDAEGAALKARLEKVSQYPDLTPN | |
| | DVATVYVRTNFSKTIWQVRANRDRYILGHKSAAVYKTLNHAITKAVGVRLNPKTTVGNIQAARTELLAAYQTAFNSPD | |
| | VKKAAsgsILAIYSTVASSLVLLVSLGAISFgsg | |

| | Encoded, for example, by |
|---|---|
| SEQ ID | the nucleotide sequence |
| NO | according to SEQ ID NO |
| 268 | 290 |
| 269 | 291 |
| 270 | 292 |
| 271 | 293 |
| 272 | 294 |
| 273 | 295 |
| 274 | 296 |
| 275 | 297 |
| 276 | 298 |
| 277 | 299 |
| 278 | 300 |
| 279 | 301 |
| 280 | 302 |
| 281 | 303 |
| 282 | 304 |
| 283 | 305 |
| 284 | 306 |
| 285 | 307 |
| 286 | 308 |
| 287 | 309 |
| 288 | 310 |

| | | Encoded, |
|---|---|---|
| | | for |
| | | example, |
| | | by the |
| | | nucleotide |
| | | sequence |
| SEQ | | according |
| ID | | to SEQ |
| NO | Amino acid sequence | ID NO |
| 368 | VAQAAIATDGKGIIDKDSRDAVINDAKLRAAIAGALVKAGFSSADAVALAPRIAKEMAKEGVLLINHHKLKALIGAQL | |
| | GLLTDAKIQRAAAAVDLGIKATLAATIHIPNALGSAAFKNAVIANLVAAGIDKHLARATAVAIVATALNPALGPIAKF | |
| | ELIKAEIAAQAALLIRRGVHLQKAAIEHVIGRAFDAAVATAIISSPILSARIVTHLVRAGIDKSIAISLAPHIVKRLA | |
| | KEPLLAFNTAKLMKAITRQIVDVITADKAIKTAEQLEKELPALDDLVKKAMGPPKPTGGGGGAGPTVTVIPVGREGGD | |
| | ITISGKGFSTTGFGVYVAVAPASVPEFYGNSDKFYGYDPSKDTTESPSTIWVYTPSQKAIGSRFAQGRPMNNDGSFTI | |
| | TMKAPPFEQGKDFVVLTTKAHGVGKGDHGDDTRTPVGYR | |
| 373 | VEPTTTISATSTHELSASDARNSIQLLNAHIATLQSVQKSVPGSDYSDQIRDLLKAAFDLRGLIETLAHGGIPFYDPS | 384, 383 |
| | TIMPRIKLVATTIDTIHTATTTLQNKVRPAHVELGLEVTKAVLLTANPASTAKELDAEGAALKARLEKVSQYPDLTPN | |
| | DVATVYVRTNFSKTIWQVRANRDRYILGHKSAAVYKTLNHAITKAVGVRLNPKTTVGNIQAARTELLAAYQTAFNSPD | |
| | VEKKAASSNRPRSVAQAAIATDGKGIIDKDSRDAVINDAKLRAAIAGALVKAGFSSADAVALAPRIAKEMAKEGVLLI | |
| | NHHKLKALIGAQLGLLTDAKIQRAAAAVDLGIKATLAATIIPNALGSAAFKNAVIANLVAAGIDKHLARATAVAIVAT | |
| | ALNPALGPIAKFELIKAEIAAQAALLIRRGVHLQKAAIEHVIGRAFDAAVATAIISSPILSARIVTHLVRAGIDKSIA | |
| | ISLAPHIVKRLAKEPLLAFNTAKLMKNITRQIVDVITADKAIKTAEQLEKELPALDDLVKKASSPPKPTPTPTPTPTA | |
| | GPTVTVIPVGREGGDITISGKGFSTTGFGVYVAVAPASVPEFYGNSDKFYGYDPSKDTTESPSTIWVYTPSQKAIGSR | |
| | FAQGRPMNNDGSFTITMKAPPFEQGKDFVVLTTKAHGVGKTDHSDDTRTPVTYREATPAPTGPKTPI | |
| 374 | VEPTTTISATSTHELSASDARNSIQLLNAHIATLQSVQKSVPGSDYSDQIRDLLKAAFDLRGLIETLAHGGIPFYDPS | 386, 387 |
| | TIMPRIKLVATTIDTIHTATTTLQNKVRPAHVELGLEVTKAVLLTANPASTAKELDAEGAALKARLEKVSQYPDLTPN | |
| | DVATVYVRTNFSKTIWQVRANRDRYILGHKSAAVYKTLNHAITKAVGVRLNPKTTVGNIQAARTELLAAYQTAFNSPD | |
| | VKKAASSNRPRSVAQAAIATDGKGIIDKDSRDAVINDAKLRAAIAGALVKAGFSSADAVALAPRIAKEMAKEGVLLIN | |
| | HHKLKALIGAQLGLLTDAKIQRAAAAVDLGIKATLAATIIPNALGSAAFKNAVIANLVAAGIDKHLARATAVAIVATA | |
| | LNPALGPIAKFELIKAEIAAQAALLIRRGVHLQKAAIEHVIGRAFDAAVATAIISSPILSARIVTHLVRAGIDKSIAI | |
| | SLAPHIVKRLAKEPLLAFNTAKLMKNITRQIVDVITADKAIKTAEQLEKELPALDDLVKKASSPPKPTPTPTPTPTAG | |
| | PTVTVIPVGREGGDITISGKGFSTTGFGVYVAVAPASVPEFYGNSDKFYGYDPSKDTTESPSTIWVYTPSQKAIGSRF | |
| | AQGRPMNNDGSFTITMKAPPFEQGKDFVVLTTKAHGVGKTDHSDDTRTPVTYREATPAPTGPKTPI | |
| 375 | VEPTTTISATSTHELSASDARNSIQLLNAHIATLQSVQKSVPGSDYSDQIRDLLKAAFDLRGLIETLAHGGIPFYDPS | 388, 389 |
| | TIMPRIKLVATTIDTIHTATTTLQNKVRPAHVELGLEVTKAVLLTANPASTAKELDAEGAALKARLEKVSQYPDLTPN | |
| | DVATSSNRPRSVAQAAIATDGKGIIDKDSRDAVINDAKLRAAIAGALVKAGFSSADAVALAPRIAKEMAKEGVLLINH | |
| | HKLKALIGAQLGLLTDAKIQRAAAAVDLGIKATLAATIIPNALGSAAFKNAVIANLVAAGIDKHLARATAVAIVATAL | |
| | NPALGPIAKFELIKAEIAAQAALLIRRGVHLQKAAIEHVIGRAFDAAVATAIISSPILSARIVTHLVRAGIDKSIAIS | |
| | LAPHIVKRLAKEPLLAFNTAKLMKNITRQIVDVITADKAIKTAEQLEKELPALDDLVKKASSPPKPTPTPTPTPTAGP | |
| | TVTVIPVGREGGDITISGKGFSTTGFGVYVAVAPASVPEFYGNSDKFYGYDPSKDTTESPSTIWVYTPSQKAIGSRFA | |
| | QGRPMNNDGSFTITMKAPPFEQGKDFVVLTTKAHGVGKTDHSDDTRTPVTYREATPAPTGPKTPI | |
| 376 | SSNRPRSVAQAAIATDGKGIIDKDSRDAVINDAKLRAAIAGALVKAGFSSADAVALAPRIAKEMAKEGVLLINHHKLK | 390, 393 |
| | ALIGAQLGLLTDAKIQRAAAAVDLGIKATLAATIIPNALGSAAFKNAVIANLVAAGIDKHLARATAVAIVATALNPAL | |
| | GPIAKFELIKAEIAAQAALLIRRGVHLQKAAIEHVIGRAFDAAVATAIISSPILSARIVTHLVRAGIDKSIAISLAPH | |
| | IVKRLAKEPLLAFNTAKLMKNITRQIVDVITADKAIKTAEQLEKELPALDDLVKKASSPPKPTPTPTPTPTAGPTVTV | |
| | IPVGREGGDITISGKGFSTTGFGVYVAVAPASVPEFYGNSDKFYGYDPSKDTTESPSTIWVYTPSQKAIGSRFAQGRP | |
| | MNNDGSFTITMKAPPFEQGKDFVVLTTKAHGVGKTDHSDDTRTPVTYREATPAPTGPKTPIVYVRTNFSKTIWQVRAN | |
| | RDRYILGHKSAAVYKTLNHAITKAVGVRLNPKTTVGNIQAARTELLAAYQTAFNSPDVKKAA | |

### Nucleotide Sequences

TABLE 7

| | | | | | the | | |
|---|---|---|---|---|---|---|---|
| | | | | | nucleotide | | |
| | | | | secretion | sequence | | |
| | | | | signal | encoding a | contains a | |
| | | | | peptide | C. acnes | TMB | Encodes the |
| | | | | sequence | antigen is | sequence | polypeptide |
| SEQ | | | | according | according | according | according |
| ID | | | Alternative | to SEQ | to SEQ ID | to SEQ | to SEQ ID |
| NO | mRNA sequence | Name | name | ID NO | NO: | ID NO | NO |
| 87 | | ssHA_C | mRNA/ | 126 | 153 | | 2 |
| | | AMP2_ | SSHA_CAMP2_ | | | | |
| | | WT_GA | WT_GA | | | | |
| 88 | | ssHA_ | mRNA/ | 127 | 154 | | 3 |
| | | CAMP2_ | SSHA_CAMP2_ | | | | |
| | | WT_GS | WT_GS | | | | |
| 89 | | ssHA_ | mRNA/HA | 128 | 155 | | 4 |
| | | CAMP2_ | SS_CAMP2_ | | | | |
| | | WT_G+ | WT | | | | |
| 90 | AUGAAGGCCAAGCU | CAMP2_ | mRNA/HA | 129 | 156 | 193 | 5 |
| | GCUGGUGCUGCUGU | WT_H | SS_CAMP2 | | | | |
| | GCACCUUCACCGCCA | A_TMB | WT_HA | | | | |
| | CCUAUGCCGUCGAG | | TMB #1 | | | | |
| | CCCACCACCACCAUC | | | | | | |
| | UCCGCCACAUCCACA | | | | | | |
| | CACGAACUGUCCGC | | | | | | |
| | AAGCGACGCCAGAA | | | | | | |
| | ACAGCAUCCAGCUG | | | | | | |
| | CUGAACGCCCACAUC | | | | | | |
| | GCCACCCUCCAGAGC | | | | | | |
| | GUGCAGAAGAGUGU | | | | | | |
| | GCCCGGCAGCGAUU | | | | | | |
| | AUAGCGACCAGAUC | | | | | | |
| | CGGGACCUGCUCAA | | | | | | |
| | GGCCGCAUUCGACC | | | | | | |
| | UGCGCGGCCUGAUC | | | | | | |
| | GAGACACUGGCUCA | | | | | | |
| | UGGCGGGAUCCCCU | | | | | | |
| | UCUAUGAUCCCAGC | | | | | | |
| | ACCAUCAUGCCCAG | | | | | | |
| | GAUCAAGCUGGUCG | | | | | | |
| | CCACUACCAUCGACA | | | | | | |
| | CCAUCCACACCGCCA | | | | | | |
| | CCACCACCCUGCAGA | | | | | | |
| | ACAAAGUGCGCCCC | | | | | | |
| | GCCCACGUGGAGCU | | | | | | |
| | GGGGCUGGAGGUGA | | | | | | |
| | CCAAAGCGGUGCUG | | | | | | |
| | CUGACAGCCAACCCU | | | | | | |
| | GCAUCCACCGCCAAG | | | | | | |
| | GAGCUGGACGCAGA | | | | | | |
| | GGGCGCUGCACUGA | | | | | | |
| | AGGCCCGGCUCGAG | | | | | | |
| | AAGGUGUCCCAGUA | | | | | | |
| | UCCCGACCUGACCCC | | | | | | |
| | CAACGACGUGGCCA | | | | | | |
| | CUGUGUACGUGCGG | | | | | | |
| | ACCAACUUCAGCAA | | | | | | |
| | GACCAUUUGGCAGG | | | | | | |
| | UCAGGGCCAAUAGA | | | | | | |
| | GACAGAUACAUCCU | | | | | | |
| | GGGGCACAAAAGCG | | | | | | |
| | CCGCCGUGUACAAG | | | | | | |
| | ACCCUGAACCACGCC | | | | | | |
| | AUCACCAAGGCCGU | | | | | | |
| | GGGAGUGCGGCUGA | | | | | | |
| | AUCCAAAGACCACC | | | | | | |
| | GUGGGCAACAUUCA | | | | | | |
| | GGCCGCCAGAACCG | | | | | | |
| | AGCUGCUGGCCGCC | | | | | | |
| | UACCAGACAGCUUU | | | | | | |
| | CAAUAGCCCCGACG | | | | | | |
| | UGAAGAAGGCCGCC | | | | | | |
| | UCCGGCAGCAUCCU | | | | | | |
| | GGCCAUCUACUCCAC | | | | | | |
| | CGUGGCCUCCAGCCU | | | | | | |
| | GGUGCUGCUGGUGA | | | | | | |
| | GCCUGGGCGCUAUC | | | | | | |
| | UCCUUCGGGUCUGG | | | | | | |
| | GUAA | | | | | | |
| 91 | aUgaaagccaagcUgcUggU | CAMP2_ | mRNA/HA | 130 | 157 | 194 | 6 |
| | gcUgcUcUgcacUUUUac | WT_HA_ | SS_CAMP2_ | | | | |
| | cgccaccUacgccgUggagcc | TMB | WT_HA | | | | |
| | caccacaacaaUcagcgccaca | | TMB #2 | | | | |
| | UccacccacgaacUgagcgcc | | | | | | |
| | UccgacgccagaaaUUccaU | | | | | | |
| | ccagcUgcUgaacgcccacaU | | | | | | |
| | cgccacccUgcagagcgUgca | | | | | | |
| | gaaaagcgUgcccggcUcUg | | | | | | |
| | aUUacagcgaccagaUccgg | | | | | | |
| | gaccUgcUgaaggccgccUU | | | | | | |
| | UgaccUgagaggccUgaUcg | | | | | | |
| | agacacUggcccacggaggca | | | | | | |
| | UcccUUUcUacgaUccUa | | | | | | |
| | gcaccaUcaUgccUcggaUc | | | | | | |
| | aagcUggUggccacaacaaUc | | | | | | |
| | gacaccaUccacaccgccacca | | | | | | |
| | ccacacUgcagaacaaagUgc | | | | | | |
| | ggccUgcccaUgUggaacUg | | | | | | |
| | ggccUggaagUgaccaaggcc | | | | | | |
| | gUgcUgcUgacagccaaUcc | | | | | | |
| | UgcUagcacagccaaggaacU | | | | | | |
| | ggaUgccgagggagccgcccU | | | | | | |
| | gaaggcUagacUggagaagg | | | | | | |
| | UgagccagUaccccgaccUga | | | | | | |
| | cccccaaUgacgUggcaaccg | | | | | | |
| | UgUacgUgagaaccaaUUU | | | | | | |
| | cagcaagaccaUcUggcagg | | | | | | |
| | UgcgggccaaUcgggacaga | | | | | | |
| | UacaUccUgggccacaagagc | | | | | | |
| | gccgccgUgUacaagacccU | | | | | | |
| | gaaUcacgccaUcaccaaggc | | | | | | |
| | cgUgggcgUgcggcUgaaU | | | | | | |
| | ccUaagaccaccgUgggcaaU | | | | | | |
| | aUccaggccgcccggacagaac | | | | | | |
| | UgcUggccgccUaccagaca | | | | | | |
| | gccUUUaaUagcccUgaUg | | | | | | |
| | Ugaagaaggccgccagcggga | | | | | | |
| | gcaUUcUggccaUcUacUc | | | | | | |
| | UaccgUggccUcUagccUg | | | | | | |
| | gUgcUgcUggUgUcUcUg | | | | | | |
| | ggagccaUUagcUUcggaU | | | | | | |
| | cUggcUgaUaa | | | | | | |
| 92 | | | mRNA/NA | | 158 | | 7 |
| | | | TMB_CAMP2_ | | | | |
| | | | WT | | | | |
| 93 | | | mRNA/NA | | 159 | | 8 |
| | | | TMB_CAMP2_ | | | | |
| | | | Nglyc- | | | | |
| | | | neg | | | | |
| 94 | | CAMP2_ | mRNA/HA | 131 | 160 | 195 | 9 |
| | | G-_HA | SS_CAMP2_ | | | | |
| | | TMB | Nglyc- | | | | |
| | | | neg_HA | | | | |
| | | | TMB | | | | |
| 95 | | | mRNA/ | | 161 | | 10 |
| | | | ssHA_CAMP2_ | | | | |
| | | | G-GA | | | | |
| 96 | | | mRNA/ | | 162 | | 11 |
| | | | ssHA_CAMP2_ | | | | |
| | | | G-GS | | | | |
| 97 | | ssHA_ | mRNA/HA | 132 | 163 | | 12 |
| | | CAMP2_ | SS_CAMP2_ | | | | |
| | | WT_G- | Nglyc-neg | | | | |
| 98 | | ssHA_ | mRNA/HA | 133 | 164 | | 13 |
| | | CAMP2_ | SS_CAMP2_ | | | | |
| | | mL | mL | | | | |
| 99 | | ssHA_ | mRNA/HA | 311 | 165 | | 14 |
| | | CAMP2 | SS_CAMP2 | | | | |
| | | G(-)_ | Nglyc- | | | | |
| | | mL | neg_mL | | | | |
| 100 | | SSHA_ | mRNA/HA | 134 | 166 | | 15 |
| | | CAMP2_ | SS_CAMP2 | | | | |
| | | mCter | mCter | | | | |
| 101 | | SSHA_ | mRNA/HA | 135 | 167 | | 16 |
| | | CAMP2_ | SS_CAMP2_ | | | | |
| | | G- | Nglyc- | | | | |
| | | mCter | neg_mCter | | | | |
| 102 | | ssHA_ | mRNA/HA | 136 | 168 | 196 | 17 |
| | | DSA1_ | SS_DSA1 | | | | |
| | | Native_ | HA TMB | | | | |
| | | TMB_WT_ | | | | | |
| | | trunc | | | | | |
| 103 | | ssHA_ | mRNA/HA | 137 | 169 | | 18 |
| | | DSA1_ | SS_DSA1_ | | | | |
| | | WT_ | WT_trunc | | | | |
| | | trunc | | | | | |
| 104 | | ssHA_ | mRNA/HA | 138 | 170 | 197 | 19 |
| | | DSA1_ | SS_DSA1_ | | | | |
| | | SGS | HA TMB- | | | | |
| | | TMB_ | sgs linker | | | | |
| | | WT_ | | | | | |
| | | trunc | | | | | |
| 105 | | ssHA_ | mRNA/HA | | 171 | | 20 |
| | | DSA2_ | SS_DSA2_ | | | | |
| | | Native_ | HA TMB | | | | |
| | | TMB_WT_ | | | | | |
| | | trunc | | | | | |
| 106 | | SSHA_ | mRNA/HA | 139 | 172 | 198 | 21 |
| | | DSA2_ | SS_DSA2_ | | | | |
| | | SGS_ | HA TMB- | | | | |
| | | TMB_WT_ | sgs linker | | | | |
| | | trunc | | | | | |
| 107 | | ssHA_ | mRNA/HA | 140 | 173 | | 22 |
| | | DSA2_ | SS_DSA2_ | | | | |
| | | WT_ | WT trunc | | | | |
| | | trunc | | | | | |
| 108 | | SSHA_ | mRNA/HA | 141 | 174 | | 23 |
| | | DSA2_ | SS_DSA2 | | | | |
| | | WT_ | mutH | | | | |
| | | trunc_ | | | | | |
| | | mutH | | | | | |
| 109 | | ssHA_ | mRNA/HA | 142 | 175 | 199 | 24 |
| | | DSA2_ | SS_DSA2- | | | | |
| | | Native_ | mutH_HA | | | | |
| | | TMB_WT_ | TMB | | | | |
| | | trunc_ | | | | | |
| | | mut | | | | | |
| 110 | | ssHA_D | mRNA/HA | 143 | 176 | 200 | 25 |
| | | SA2_S | SS_DSA2- | | | | |
| | | GS_TMB | mutH_HA | | | | |
| | | WT_ | TMB-sgs | | | | |
| | | trunc_ | linker | | | | |
| | | mut | | | | | |
| 111 | | | mRNA/HA | 137 | 177 | 200 | 26 |
| | | | SS_DSA2- | | | | |
| | | | Nglyc- | | | | |
| | | | neg_HA | | | | |
| | | | TMB-sgs | | | | |
| | | | linker | | | | |
| 112 | | | mRNA/HA | 137 | 178 | 312 | 27 |
| | | | SS_DSA2- | | | | |
| | | | Nglyc- | | | | |
| | | | neg_HA | | | | |
| | | | TMB | | | | |
| 113 | AUGAAGGCCAAGCU | SSHA_ | mRNA/ | 144 | 179 | | 28 |
| | GCUGGUGCUGCUGU | H4_V3 | ssHA H4- | | | | |
| | GCACAUUCACAGCA | | V3 | | | | |
| | ACCUAUGCAUCAUC | | | | | | |
| | CAACAGGCCUCGCUC | | | | | | |
| | CGUGGCCCAGGCUG | | | | | | |
| | CUAUUGCUACCGAC | | | | | | |
| | GGCAAGGGAAUCAU | | | | | | |
| | CGAUAAAGACUCCA | | | | | | |
| | GGGAUGCCGUGAUC | | | | | | |
| | AACGAUGCCAAGCU | | | | | | |
| | CCGGGCCGCAAUUG | | | | | | |
| | CCGGAGCCCUGGUG | | | | | | |
| | AAAGCCGGGUUCAG | | | | | | |
| | CUCCGCCGAUGCUG | | | | | | |
| | UGGCUCUGGCUCCA | | | | | | |
| | AGGAUUGCUAAGGA | | | | | | |
| | GAUGGCCAAAGAGG | | | | | | |
| | GGGUGCUGCUGAUU | | | | | | |
| | AACCACCACAAGCU | | | | | | |
| | GAAGGCACUGAUCG | | | | | | |
| | GAGCACAGCUGGGA | | | | | | |
| | CUGCUGACUGACGC | | | | | | |
| | CAAAAUCCAGCGCG | | | | | | |
| | CCGCUGCAGCAGUC | | | | | | |
| | GACCUGGGCAUCAA | | | | | | |
| | GGCCACACUGGCUG | | | | | | |
| | CUACCAUCAUUCCU | | | | | | |
| | AACGCUCUGGGCAG | | | | | | |
| | CGCAGCCUUUAAGA | | | | | | |
| | AUGCCGUUAUCGCC | | | | | | |
| | AAUCUCGUGGCUGC | | | | | | |
| | UGGAAUUGAUAAGC | | | | | | |
| | ACCUGGCCAGAGCC | | | | | | |
| | ACAGCUGUGGCCAU | | | | | | |
| | CGUGGCCACUGCCCU | | | | | | |
| | GAACCCAGCAUUGG | | | | | | |
| | GCCCUAUCGCUAAG | | | | | | |
| | UUUGAGCUGAUUAA | | | | | | |
| | GGCCGAGAUCGCCG | | | | | | |
| | CUCAGGCUGCCCUGC | | | | | | |
| | UGAUUCGCAGGGGA | | | | | | |
| | GUGCACCUGCAGAA | | | | | | |
| | GGCUGCCAUCGAGC | | | | | | |
| | ACGUCAUCGGCAGA | | | | | | |
| | GCAUUCGACGCCGC | | | | | | |
| | UGUCGCCACUGCUA | | | | | | |
| | UCAUCUCUAGCCCA | | | | | | |
| | AUCCUGAGCGCCAG | | | | | | |
| | GAUCGUCACUCACC | | | | | | |
| | UGGUGAGAGCUGGG | | | | | | |
| | AUUGACAAAUCCAU | | | | | | |
| | CGCUAUCAGCCUGG | | | | | | |
| | CCCCUCACAUCGUGA | | | | | | |
| | AGAGGCUGGCUAAG | | | | | | |
| | GAACCACUGCUGGC | | | | | | |
| | UUUUAACACUGCUA | | | | | | |
| | AGCUGAUGAAGAAU | | | | | | |
| | AUUACAAGACAGAU | | | | | | |
| | CGUGGACGUCAUCA | | | | | | |
| | CCGCCGACAAAGCCA | | | | | | |
| | UCAAGACAGCCGAG | | | | | | |
| | CAGCUCGAGAAGGA | | | | | | |
| | GCUGCCUGCCCUGG | | | | | | |
| | AUGACCUGGUGAAG | | | | | | |
| | AAGGCAUCCUCCCCC | | | | | | |
| | CCUAAGCCAACCCCU | | | | | | |
| | ACCCCAACCCCUACA | | | | | | |
| | CCUACUUAA | | | | | | |
| 114 | | | mRNA/ | 137 | 181 | | 30 |
| | | | ssHA_H4- | | | | |
| | | | V3 Nglyc- | | | | |
| | | | neg | | | | |
| 115 | AUGAAGGCAAAGCU | ssHA_ | mRNA/ | 145 | 182 | | 31 |
| | GCUGGUGCUGCUGU | P028_ | P28-V7 | | | | |
| | GCACAUUUACUGCC | V7 | | | | | |
| | ACUUACGCAGCAGG | | | | | | |
| | CCCUACCGUCACAGU | | | | | | |
| | GAUCCCUGUGGGCA | | | | | | |
| | GAGAAGGUGGCGAC | | | | | | |
| | AUCACCAUCAGCGG | | | | | | |
| | CAAGGGCUUCAGCA | | | | | | |
| | CCACAGGCUUUGGC | | | | | | |
| | GUGUACGUGGCCGU | | | | | | |
| | GGCACCUGCUAGCG | | | | | | |
| | UGCCCGAAUUCUAC | | | | | | |
| | GGCAAUAGCGACAA | | | | | | |
| | GUUCUACGGCUACG | | | | | | |
| | ACCCCAGCAAGGAC | | | | | | |
| | ACCACCGAGAGCCCU | | | | | | |
| | AGCACCAUCUGGGU | | | | | | |
| | GUACACCCCUAGCCA | | | | | | |
| | GAAGGCCAUCGGCU | | | | | | |
| | CUCGGUUCGCCCAG | | | | | | |
| | GGCAGACCCAUGAA | | | | | | |
| | CAAUGACGGCAGCU | | | | | | |
| | UCACAAUCACCAUG | | | | | | |
| | AAGGCCCCCCCUUUC | | | | | | |
| | GAGCAGGGCAAAGA | | | | | | |
| | CUUCGUGGUGCUGA | | | | | | |
| | CCACAAAGGCCCAU | | | | | | |
| | GGCGUGGGCAAAAC | | | | | | |
| | CGAUCACAGCGACG | | | | | | |
| | ACACACGGACACCU | | | | | | |
| | GUCACCUACAGAGA | | | | | | |
| | GGCUACCCCUGCCCC | | | | | | |
| | UACAGGCCCUAAGA | | | | | | |
| | CACCCAUCGCCCCUA | | | | | | |
| | GCAAGCAGCCUAGC | | | | | | |
| | AAGCAGGCCGCCCCU | | | | | | |
| | AGCAAGCAGGUGAA | | | | | | |
| | GCCUUCUAAGCAGG | | | | | | |
| | CCGGCCCCAACAAGC | | | | | | |
| | AGUCCACCACACCUC | | | | | | |
| | AGCAGAAGACAGCC | | | | | | |
| | GAGCACAGAAGCCA | | | | | | |
| | GACCCCUGCCGCCCA | | | | | | |
| | CCGGACCAUGACAA | | | | | | |
| | AGCAGGUGAGCACC | | | | | | |
| | AUCGGCGCCAGCAA | | | | | | |
| | GGUGACAAGCGGCU | | | | | | |
| | CUCUGACCUGGGGC | | | | | | |
| | AUCAGAACCUCUUU | | | | | | |
| | UACCUCCUACCUGA | | | | | | |
| | GAGGCCCUAUCGCC | | | | | | |
| | AACGGCUCUUGGAA | | | | | | |
| | GCUGAGCGGAGGCG | | | | | | |
| | CCAAUUGGAAUGGC | | | | | | |
| | UCCGCCUUCACCUUC | | | | | | |
| | CCUCUGACCAGCGGC | | | | | | |
| | AGCUUCGACCCAGCC | | | | | | |
| | ACCAAGUCCGGAAG | | | | | | |
| | CCUGAAGUACAGCG | | | | | | |
| | GCAGCGUGCACAUG | | | | | | |
| | ACAGGCCACCACGGC | | | | | | |
| | AUCCUGGACAUGAC | | | | | | |
| | CCUGGCCGAGCCAA | | | | | | |
| | GCCUGCAGAUCAAG | | | | | | |
| | GGAAGCACCGGCCA | | | | | | |
| | CCUGUAUCUGGACG | | | | | | |
| | UGAAAAGCAGCAGC | | | | | | |
| | AUGGACGGCAAGAA | | | | | | |
| | GACCAAUUACGGCA | | | | | | |
| | GAGUGGACUUCGCC | | | | | | |
| | ACCUUCGGAGUGAG | | | | | | |
| | CGUGUCUGGCAACG | | | | | | |
| | CCGCCAUCAAGGGC | | | | | | |
| | AGCCCUGUGAAGCU | | | | | | |
| | GACCGCCACAGGCGC | | | | | | |
| | UAAGGCUUUUGCCG | | | | | | |
| | GCUUCUACAGAGCC | | | | | | |
| | GGCGAGCCUAUGAA | | | | | | |
| | CCCUCUGAGCACCAA | | | | | | |
| | UCUGACCCUGAGCG | | | | | | |
| | CCGAGAAGGUGUCC | | | | | | |
| | CACAACGUGACCGU | | | | | | |
| | GGACGCAGUGACAG | | | | | | |
| | GCAAGGUGAUCGGC | | | | | | |
| | GACGACUCUGGCAA | | | | | | |
| | GGGAGCCGGCCGGG | | | | | | |
| | GCCUGCCCGUGACA | | | | | | |
| | UAA | | | | | | |
| 117 | | SSHA_ | mRNA/ | 146 | 184 | | 33 |
| | | PITP_ | SSHA_PITP | | | | |
| | | WT | WT_trunc | | | | |
| | | trunc | | | | | |
| 118 | | | mRNA/ | 137 | 185 | | 34 |
| | | | SSHA_PITP | | | | |
| | | | Nglyc-neg | | | | |
| 119 | | P028_ | mRNA/ | 147 | 186 | | 35 |
| | | V7_ | ssHA_P28- | | | | |
| | | Glyneg | V7_Nglyc- | | | | |
| | | | neg | | | | |
| 120 | | ssHA_ | mRNA/ | 148 | 187 | 201 | 36 |
| | | PITP_ | SSHA_PITP_ | | | | |
| | | SGS_ | WT_HA | | | | |
| | | TMB_ | TMB-sgs | | | | |
| | | HA_WT_ | linker | | | | |
| | | trunc | | | | | |
| 121 | | | mRNA/ | 137 | 188 | 200 | 37 |
| | | | SSHA_PITP | | | | |
| | | | Nglyc- | | | | |
| | | | neg_HA | | | | |
| | | | TMB-sgs | | | | |
| | | | linker | | | | |
| 122 | aUgaaggccaaacUgcUggU | nCO- | H4-V3-f- | 149 | 189 | | 38 |
| | gcUgcUgUgUaccUUUac | ssHA_ | PRO-028- | | | | |
| | agccacaUaUgccUccUcUa | H4-V3- | V7-F16, | | | | |
| | aUagaccaagaUccgUggcU | f-PRO- | triple | | | | |
| | caggccgccaUcgcUacagaU | 028- | chimera | | | | |
| | gggaaaggaaUcaUcgaUaa | V7- | P22/P27P28 | | | | |
| | ggaUagUcgggaUgcagUg | F16 | | | | | |
| | aUcaacgacgccaaacUccga | | | | | | |
| | gcUgccaUcgccggcgcccU | | | | | | |
| | ggUgaaggcUgggUUUUc | | | | | | |
| | cagcgccgacgcagUggcccU | | | | | | |
| | ggcaccUagaaUcgccaagga | | | | | | |
| | gaUggcaaaggagggcgUcc | | | | | | |
| | UgcUgaUUaaUcaccacaag | | | | | | |
| | cUgaaagcUcUgaUUgggg | | | | | | |
| | cUcagcUcggacUgcUgac | | | | | | |
| | UgacgccaagaUccagagggc | | | | | | |
| | cgcagcUgccgUggaUcUgg | | | | | | |
| | ggaUcaaggccacUcUggcc | | | | | | |
| | gcUacUaUUaUUccUaac | | | | | | |
| | gcUcUggggUcUgccgccU | | | | | | |
| | UcaagaaUgcUgUcaUUgc | | | | | | |
| | caaccUggUggcUgcUggca | | | | | | |
| | UcgacaaacaccUcgccaggg | | | | | | |
| | cUaccgcUgUggccaUcgU | | | | | | |
| | UgccacagcccUgaaUccUg | | | | | | |
| | cccUgggcccaaUcgcUaaa | | | | | | |
| | UUUgagcUgaUUaaggcU | | | | | | |
| | gagaUcgccgcccaggccgcac | | | | | | |
| | UgcUgaUcagaaggggggU | | | | | | |
| | ccaUcUgcagaaggccgcUa | | | | | | |
| | UUgaacaUgUgaUcggccg | | | | | | |
| | cgcaUUcgacgcUgccgUgg | | | | | | |
| | ccaccgcUaUcaUUUccUc | | | | | | |
| | UccaaUccUgagcgcacgga | | | | | | |
| | UUgUgacccaccUggUgag | | | | | | |
| | ggcUggaaUUgaUaaaagca | | | | | | |
| | UcgcUaUcUcccUggcUcc | | | | | | |
| | UcaUaUcgUgaagcgccUg | | | | | | |
| | gccaaggagccccUgcUggc | | | | | | |
| | UUUcaacaccgccaagcUga | | | | | | |
| | UgaagaaUaUUacaagacag | | | | | | |
| | aUcgUUgacgUgaUcacag | | | | | | |
| | ccgaUaaagccaUcaaaacag | | | | | | |
| | cUgagcagcUggaaaaggagc | | | | | | |
| | UccccgcccUggacgaccUg | | | | | | |
| | gUcaaaaaagcUagcUcccc | | | | | | |
| | UccUaagccUacccccacacc | | | | | | |
| | UacUccaacccccaccgccgg | | | | | | |
| | gcccaccgUgacagUgaUccc | | | | | | |
| | cgUgggaagggaggggggcga | | | | | | |
| | caUUaccaUUagcggaaaag | | | | | | |
| | ggUUcUccacaaccgggUU | | | | | | |
| | cggcgUcUacgUggccgUg | | | | | | |
| | gcUccUgccUcUgUgccag | | | | | | |
| | agUUcUaUggcaaUagcga | | | | | | |
| | caagUUcUaUgggUacgaU | | | | | | |
| | ccaUcUaaggacaccaccgaa | | | | | | |
| | agUcccagcaccaUcUgggU | | | | | | |
| | gUaUacaccaUcccagaaagc | | | | | | |
| | aaUcggcagcagaUUcgccca | | | | | | |
| | aggcaggcccaUgaaUaaUg | | | | | | |
| | aUggcUcUUUcaccaUUa | | | | | | |
| | caaUgaaggccccUccUUUc | | | | | | |
| | gagcagggaaaggaUUUcgU | | | | | | |
| | ggUgcUgaccaccaaagcaca | | | | | | |
| | UggcgUUggaaagaccgacc | | | | | | |
| | acagcgacgaUacUcggaccc | | | | | | |
| | cUgUgacUUacagagaggcc | | | | | | |
| | accccUgcUccaaccggcccU | | | | | | |
| | aaaacUcccaUcUgaUaa | | | | | | |
| 123 | | nCO- | H4-V3 low | 150 | 190 | | 39 |
| | | ssHA_ | glyc | | | | |
| | | PRO-H4- | | | | | |
| | | V3- | | | | | |
| | | lowglyc | | | | | |
| 124 | aUgaaggccaagcUgcUggU | gc.CO- | PRO-028- | 151 | 191 | | 40 |
| | gcUgcUgUgcaccUUcacc | ssHA_ | V7-C12 low | | | | |
| | gccaccUacgccUccagcaac | PRO- | glyc | | | | |
| | cggccUagaagcgUggcccag | 028- | | | | | |
| | gccgccaUcgccaccgacggca | V12- | | | | | |
| | agggcaUcaUcgacaaggaU | lowglyc | | | | | |
| | UcccgggacgccgUgaUcaa | | | | | | |
| | UgacgccaagcUgagagccgc | | | | | | |
| | caUcgccggcgcccUggUga | | | | | | |
| | aggccggcUUcagcagcgccg | | | | | | |
| | acgccgUggcccUggccccca | | | | | | |
| | gaaUcgccaaggagaUggcca | | | | | | |
| | aggaaggcgUgcUgcUgaUc | | | | | | |
| | aaUcaccacaagcUgaaggcc | | | | | | |
| | cUgaUcggcgcccagcUggg | | | | | | |
| | ccUgcUgacagaUgccaaga | | | | | | |
| | UccagagagccgcUgccgccg | | | | | | |
| | UggaccUgggcaUcaaggcc | | | | | | |
| | acccUggccgccaccaUcaUc | | | | | | |
| | ccUaacgcccUgggcagcgc | | | | | | |
| | UgccUUcaagaaUgccgUg | | | | | | |
| | aUcgccaaccUggUggccgc | | | | | | |
| | cggcaUcgacaagcaccUggc | | | | | | |
| | cagagccacagccgUggccaU | | | | | | |
| | cgUggccaccgcccUgaaUcc | | | | | | |
| | UgcccUgggccccaUcgccaa | | | | | | |
| | gUUcgagcUgaUcaaggccg | | | | | | |
| | agaUcgccgcccaggccgccc | | | | | | |
| | UgcUgaUccggcggggcgU | | | | | | |
| | gcaccUgcagaaggccgccaU | | | | | | |
| | cgaacacgUgaUcggcagagc | | | | | | |
| | cUUcgaUgccgccgUggcca | | | | | | |
| | ccgccaUcaUcagcUccccca | | | | | | |
| | UccUgagcgccagaaUcgUg | | | | | | |
| | acccaccUggUgcgggccggc | | | | | | |
| | aUcgacaagUccaUcgccaU | | | | | | |
| | UagccUggccccUcacaUcg | | | | | | |
| | UgaagcgccUggccaaggagc | | | | | | |
| | cccUgcUggccUUcaaUacc | | | | | | |
| | gccaagcUgaUgaagaaUaU | | | | | | |
| | caccagacagaUcgUggaUg | | | | | | |
| | UgaUcaccgccgaUaaggcca | | | | | | |
| | UcaagaccgccgagcagcUgg | | | | | | |
| | agaaagagcUgcccgcccUgg | | | | | | |
| | acgaccUggUgaaaaaggcca | | | | | | |
| | UgggccccccUaagccUacag | | | | | | |
| | gcggcggcggaggcgcUggac | | | | | | |
| | cUaccgUgaccgUgaUcccc | | | | | | |
| | gUgggcagagagggcggcga | | | | | | |
| | UaUcaccaUcagcggcaagg | | | | | | |
| | gcUUcagcaccaccggcUUc | | | | | | |
| | ggcgUgUacgUggccgUgg | | | | | | |
| | ccccUgccagcgUgcccgag | | | | | | |
| | UUcUacggcaaUagcgacaa | | | | | | |
| | gUUcUacggcUacgacccca | | | | | | |
| | gcaaggacaccacagaaUcUc | | | | | | |
| | ccagcaccaUcUgggUgUac | | | | | | |
| | acccccagccagaaggccaUU | | | | | | |
| | ggcagccggUUcgcccagggc | | | | | | |
| | cggccUaUgaacaaUgaUgg | | | | | | |
| | cagcUUcacaaUcaccaUga | | | | | | |
| | aggccccacccUUcgagcagg | | | | | | |
| | gcaaggacUUcgUggUgcU | | | | | | |
| | gaccaccaaggcccacggagUg | | | | | | |
| | ggcaagggcgaccacggcgacg | | | | | | |
| | acacacggacccccgUgggcU | | | | | | |
| | acagaUgaUaa | | | | | | |
| 125 | aUgaaagccaagcUgcUggU | nCO- | PRO-028- | 152 | 192 | | 41 |
| | gcUgcUgUgUacUUUUac | ssHA_ | V7-C12, | | | | |
| | UgcaaccUacgccagcagcaa | PRO- | triple | | | | |
| | ccgcccaaggUcUgUcgcaca | 028- | chimera | | | | |
| | ggcUgcUaUcgccacagaUg | V7- | P22/P27/ | | | | |
| | gcaagggaaUcaUUgaUaag | C12 | P28 | | | | |
| | gacagcagggaUgcUgUgaU | | | | | | |
| | caacgacgcaaaacUgagagcc | | | | | | |
| | gccaUcgccggcgcacUggU | | | | | | |
| | gaaagccggcUUUUcUUcc | | | | | | |
| | gccgacgcagUggcUcUggc | | | | | | |
| | UcccagaaUcgcUaaggaaa | | | | | | |
| | UggccaaggagggagUgcUg | | | | | | |
| | cUgaUcaaUcaccacaaacU | | | | | | |
| | gaaggcUcUgaUcggggcUc | | | | | | |
| | agcUgggccUgcUgacUgac | | | | | | |
| | gcUaagaUUcagagagcagc | | | | | | |
| | agcagcUgUcgaccUgggaa | | | | | | |
| | UcaaggcaacccUggcagcca | | | | | | |
| | cUaUcaUUccaaaUgcUcU | | | | | | |
| | gggcUcUgccgccUUcaaaa | | | | | | |
| | acgccgUgaUUgccaaUcU | | | | | | |
| | cgUggcagcUggaaUcgaUa | | | | | | |
| | agcaccUggcacgggccaccgc | | | | | | |
| | cgUggcUaUcgUggccaccg | | | | | | |
| | cUcUgaaUcccgcccUgggc | | | | | | |
| | ccUaUUgccaagUUUgaac | | | | | | |
| | UgaUcaaagccgagaUcgcU | | | | | | |
| | gcacaggccgcccUgcUcaUc | | | | | | |
| | cgcaggggggUccaccUgcag | | | | | | |
| | aaagcagccaUcgagcacgUg | | | | | | |
| | aUcggcagggccUUUgaUg | | | | | | |
| | cagccgUcgccacUgccaUca | | | | | | |
| | UcagcUcUccaaUccUgUc | | | | | | |
| | UgcccggaUUgUgacUcac | | | | | | |
| | cUggUgcgcgccggcaUcga | | | | | | |
| | UaagUccaUcgcaaUcUcU | | | | | | |
| | cUggccccacacaUcgUgaag | | | | | | |
| | agacUggccaaagaaccacUc | | | | | | |
| | cUggcUUUcaaUaccgccaa | | | | | | |
| | acUcaUgaaaaacaUcacccg | | | | | | |
| | gcagaUcgUcgaUgUgaUU | | | | | | |
| | accgccgacaaggcUaUcaag | | | | | | |
| | accgccgagcagcUcgaaaagg | | | | | | |
| | agcUgccUgcccUggacgac | | | | | | |
| | cUggUgaagaaagccUccUc | | | | | | |
| | UccacccaaacccaccccaacU | | | | | | |
| | ccaacUccaaccccUacagcU | | | | | | |
| | ggccccaccgUgaccgUcaUc | | | | | | |
| | ccagUgggaagggaaggagga | | | | | | |
| | gacaUcacaaUcUcUggaaa | | | | | | |
| | ggggUUUagcacaaccggaU | | | | | | |
| | UUggagUgUacgUcgccg | | | | | | |
| | UggcccccgcaUccgUgcca | | | | | | |
| | gaaUUcUacggcaaUUccg | | | | | | |
| | aUaaaUUcUacgggUacga | | | | | | |
| | ccccUccaaggacacUacaga | | | | | | |
| | gagccccUcUacaaUUUgg | | | | | | |
| | gUgUacaccccaagccagaag | | | | | | |
| | gccaUcggaagcagaUUUgc | | | | | | |
| | UcagggccggccaaUgaacaa | | | | | | |
| | cgacggaUccUUcacaaUca | | | | | | |
| | caaUgaaagcaccUcccUUU | | | | | | |
| | gagcagggcaaggaUUUUg | | | | | | |
| | UggUgcUgaccaccaaggcU | | | | | | |
| | cacggggUcggaaagaccgaU | | | | | | |
| | cacagcgacgaUacaagaaccc | | | | | | |
| | ccgUgacaUaccggUgaUaa | | | | | | |

| | | | the | | |
|---|---|---|---|---|---|
| | | | nucleotide | | |
| | | | sequence | contains | |
| | | | encoding a | a TMB | Encodes the |
| | | | C. acnes | sequence | polypeptide |
| SEQ | | | antigen is | according | according |
| ID | | | according to | to SEQ | to SEQ ID |
| NO | mRNA sequence | Name | SEQ ID NO: | ID NO | NO |
| 377 | AUGAAGGCCAAGCUGCUGGUGCUGCUGUG | Quad- | 384 | 395 | 369 |
| | UACCUUCACUGCAACAUAUGCUGUGGAGC | CAMP2- | | | |
| | CCACAACCACAAUCUCUGCCACAAGCACCC | H4V3- | | | |
| | ACGAGCUGUCUGCCAGCGACGCCAGAAAC | f-PRO- | | | |
| | UCCAUCCAGCUGCUGAAUGCCCACAUCGCC | 028-V7- | | | |
| | ACACUGCAGAGCGUGCAGAAAUCCGUGCC | F16_sgs_HA- | | | |
| | UGGCAGCGAUUACAGCGACCAGAUCCGGG | TM_gsg_ssHA | | | |
| | ACCUGCUGAAGGCCGCCUUUGAUCUGAGA | codon | | | |
| | GGCCUGAUCGAAACCCUGGCCCACGGCGGC | optimization #1 | | | |
| | GACACCAUCCACACAGCUACAACCACCCUG | | | | |
| | CAGAACAAGGUGCGGCCAGCCCAUGUGGA | | | | |
| | AUUCCCUUCUAUGACCCCUCCACCAUCAUG | | | | |
| | CCCAGAAUCAAGCUGGUGGCCACCACCAUC | | | | |
| | ACUGGGCCUGGAGGUGACAAAGGCCGUGC | | | | |
| | UGCUGACCGCCAAUCCUGCCAGCACAGCAA | | | | |
| | AAGAACUGGACGCCGAGGGAGCUGCUCUG | | | | |
| | AAGGCCAGACUGGAAAAGGUGAGCCAGUA | | | | |
| | CCCCGACCUGACACCUAAUGAUGUGGCCAC | | | | |
| | AGUGUACGUGAGAACCAAUUUCAGCAAGA | | | | |
| | CCAUCUGGCAGGUGAGAGCCAACAGAGAU | | | | |
| | CGGUACAUCCUGGGCCACAAGUCCGCCGCC | | | | |
| | GUGUACAAGACCCUGAAUCACGCCAUCACC | | | | |
| | AAGGCCGUGGGCGUGCGGCUGAAUCCUAA | | | | |
| | GACCACCGUGGGCAACAUCCAGGCCGCCAG | | | | |
| | AACCGAACUGCUGGCCGCCUACCAGACCGC | | | | |
| | CUUCAACAGCCCCGAUGUGAAGAAGGCCG | | | | |
| | CUAGCUCCAAUAGACCCAGAUCCGUGGCCC | | | | |
| | AGGCCGCCAUCGCCACAGAUGGCAAGGGG | | | | |
| | AUCAUCGACAAGGAUUCCAGAGACGCUGU | | | | |
| | GAUCAACGACGCAAAGCUGAGAGCCGCCA | | | | |
| | UCGCCGGAGCUCUGGUGAAGGCCGGCUUU | | | | |
| | AGCAGCGCCGACGCCGUGGCACUGGCCCCC | | | | |
| | AGAAUUGCAAAGGAGAUGGCAAAGGAGGG | | | | |
| | CGUGCUGCUGAUCAAUCACCACAAGCUGA | | | | |
| | AGGCCCUGAUCGGCGCACAGCUGGGCCUGC | | | | |
| | UGACCGACGCAAAGAUUCAGAGAGCCGCC | | | | |
| | GCAGCCGUGGACCUGGGCAUCAAGGCCACC | | | | |
| | CUGGCUGCCACCAUCAUCCCUAACGCCCUG | | | | |
| | GGCAGCGCCGCCUUCAAGAAUGCAGUGAU | | | | |
| | CGCCAACCUGGUGGCCGCCGGAAUCGACAA | | | | |
| | GCACCUGGCCAGAGCCACAGCCGUCGCUAU | | | | |
| | CGUGGCCACAGCCCUGAAUCCCGCCCUGGG | | | | |
| | CCCCAUCGCCAAAUUUGAACUGAUCAAGG | | | | |
| | CAGAAAUCGCUGCCCAGGCCGCCCUGCUCA | | | | |
| | UCAGAAGAGGCGUCCAUCUGCAGAAGGCC | | | | |
| | GCCAUCGAGCACGUGAUUGGCAGAGCCUU | | | | |
| | CGACGCCGCCGUGGCCACAGCUAUCAUUUC | | | | |
| | UUCCCCUAUCCUGUCUGCCAGAAUCGUGAC | | | | |
| | ACACCUGGUGAGAGCCGGAAUCGACAAGU | | | | |
| | CCAUCGCCAUCUCCCUGGCCCCCCACAUCG | | | | |
| | UGAAGAGACUGGCUAAGGAGCCUCUGCUG | | | | |
| | GCCUUCAAUACAGCCAAGCUGAUGAAGAA | | | | |
| | CAUCACAAGACAGAUCGUGGACGUGAUCA | | | | |
| | CCGCCGACAAGGCCAUCAAGACAGCCGAGC | | | | |
| | AGCUGGAGAAGGAACUGCCAGCCCUGGAC | | | | |
| | GACCUGGUGAAGAAGGCCAGCAGCCCUCCC | | | | |
| | AAGCCUACCCCUACCCCAACCCCUACACCU | | | | |
| | ACAGCCGGACCUACCGUGACCGUGAUCCCU | | | | |
| | GUGGGCAGAGAGGGCGGCGACAUCACCAU | | | | |
| | CAGCGGCAAGGGCUUCAGCACAACAGGCU | | | | |
| | UUGGCGUGUACGUGGCCGUGGCCCCAGCC | | | | |
| | AGCGUGCCUGAGUUUUACGGCAAUAGCGA | | | | |
| | CAAGUUCUACGGCUACGACCCCUCCAAGGA | | | | |
| | CACCACCGAGAGCCCAAGCACCAUCUGGGU | | | | |
| | GUACACACCUAGCCAGAAGGCCAUCGGCA | | | | |
| | GCAGAUUUGCCCAGGGCAGGCCUAUGAAU | | | | |
| | AAUGACGGCAGCUUCACAAUCACAAUGAA | | | | |
| | GGCCCCCCCCUUUGAGCAGGGCAAGGACUU | | | | |
| | CGUGGUGCUGACCACCAAGGCCCACGGCGU | | | | |
| | GGGCAAGACAGAUCACAGCGACGACACCC | | | | |
| | GGACCCCCGUGACCUACAGAGAAGCAACAC | | | | |
| | CUGCCCCUACCGGGCCUAAGACACCCAUCA | | | | |
| | GCGGCAGCAUCCUGGCUAUCUACAGCACA | | | | |
| | GUGGCUUCUAGCCUGGUGCUGCUGGUGUC | | | | |
| | UCUGGGCGCCAUCAGCUUUGGCAGCGGCU | | | | |
| | GAUAA | | | | |
| 378 | AUGAAGGCUAAGCUGCUGGUGCUGCUGUG | Quad- | 385 | 396 | 369 |
| | CACAUUCACAGCCACCUACGCCGUGGAGCC | CAMP2- | | | |
| | UACCACCACAAUCAGCGCCACCAGCACCCA | H4V3- | | | |
| | CGAGCUGAGCGCCAGCGAUGCCAGAAAUA | f-PRO- | | | |
| | GCAUCCAGCUGCUGAAUGCCCACAUCGCCA | 028-V7- | | | |
| | CCCUGCAGUCCGUGCAGAAAAGCGUGCCU | F16_sgs | | | |
| | GGAAGCGAUUACAGCGAUCAGAUCAGGGA | HA- | | | |
| | CCUGCUGAAGGCCGCCUUCGAUCUGAGAG | TM_gsg_ | | | |
| | GCCUGAUCGAGACACUGGCCCACGGCGGCA | ssHA | | | |
| | UCCCUUUCUACGACCCCAGCACCAUCAUGC | codon | | | |
| | CUAGAAUCAAACUGGUGGCCACCACCAUC | optimization #2 | | | |
| | GACACCAUCCACACCGCAACAACAACCCUG | | | | |
| | CAGAAUAAGGUGCGGCCUGCCCACGUGGA | | | | |
| | ACUGGGACUGGAAGUGACAAAGGCCGUGC | | | | |
| | UGCUGACCGCUAAUCCCGCCUCCACCGCCA | | | | |
| | AGGAGCUGGAUGCCGAAGGAGCUGCCCUG | | | | |
| | AAAGCCAGACUGGAGAAGGUGAGCCAGUA | | | | |
| | CCCUGAUCUGACCCCUAACGACGUGGCCAC | | | | |
| | CGUGUACGUGAGAACCAAUUUCAGCAAGA | | | | |
| | CCAUCUGGCAGGUGAGAGCCAAUCGGGAC | | | | |
| | AGAUACAUCCUGGGCCACAAGUCUGCCGCC | | | | |
| | GUGUAUAAAACCCUGAAUCACGCCAUCAC | | | | |
| | CAAGGCCGUGGGCGUGAGACUGAACCCUA | | | | |
| | AGACAACCGUGGGCAACAUCCAGGCCGCCA | | | | |
| | GAACAGAGCUGCUGGCCGCCUACCAGACA | | | | |
| | GCCUUCAAUAGCCCCGACGUGAAGAAGGC | | | | |
| | CGCUUCCAGCAAUAGACCCCGGAGCGUGGC | | | | |
| | ACAGGCUGCUAUUGCCACCGACGGAAAGG | | | | |
| | GCAUCAUCGAUAAAGACAGCCGGGAUGCC | | | | |
| | GUGAUCAAUGACGCCAAGCUGAGAGCAGC | | | | |
| | AAUCGCCGGCGCUCUGGUGAAGGCAGGCU | | | | |
| | UCAGCAGCGCCGACGCCGUGGCUCUGGCCC | | | | |
| | CUAGAAUCGCUAAGGAGAUGGCCAAGGAG | | | | |
| | GGCGUGCUGCUGAUCAAUCACCACAAGCU | | | | |
| | GAAGGCCCUGAUCGGCGCCCAGCUGGGAC | | | | |
| | UGCUGACAGAUGCCAAGAUCCAGAGAGCC | | | | |
| | GCUGCAGCCGUGGAUCUGGGCAUCAAGGC | | | | |
| | CACACUGGCCGCCACCAUCAUCCCAAACGC | | | | |
| | CCUGGGCAGCGCCGCCUUCAAAAAUGCCGU | | | | |
| | GAUUGCCAACCUGGUGGCCGCCGGCAUCG | | | | |
| | AUAAGCACCUGGCCAGAGCCACAGCCGUG | | | | |
| | GCCAUCGUGGCCACAGCCCUGAAUCCCGCA | | | | |
| | CUGGGACCUAUUGCCAAGUUCGAACUCAU | | | | |
| | UAAGGCCGAAAUCGCAGCCCAAGCCGCCCU | | | | |
| | GCUGAUUCGGCGGGGCGUGCACCUGCAGA | | | | |
| | AAGCCGCCAUUGAGCACGUGAUCGGCAGA | | | | |
| | GCCUUCGAUGCCGCUGUGGCCACCGCCAUC | | | | |
| | AUCUCCUCUCCUAUCCUGUCUGCUAGAAUC | | | | |
| | GUGACCCACCUGGUCAGAGCCGGAAUCGA | | | | |
| | CAAGUCUAUCGCUAUCAGCCUGGCCCCCCA | | | | |
| | CAUCGUGAAGAGACUGGCCAAAGAGCCCC | | | | |
| | UGCUGGCCUUCAACACAGCCAAGCUCAUG | | | | |
| | AAGAAUAUCACCAGACAGAUCGUGGACGU | | | | |
| | GAUCACCGCCGACAAGGCCAUCAAAACCGC | | | | |
| | UGAGCAGCUGGAGAAGGAGCUGCCCGCCC | | | | |
| | UGGAUGACCUGGUGAAGAAGGCCAGCAGC | | | | |
| | CCCCCCAAGCCUACACCUACACCUACCCCA | | | | |
| | ACCCCUACAGCCGGCCCUACAGUGACCGUG | | | | |
| | AUUCCUGUGGGCAGAGAAGGAGGCGACAU | | | | |
| | CACCAUCAGCGGAAAGGGCUUCUCUACCAC | | | | |
| | AGGCUUCGGCGUGUACGUGGCCGUGGCCC | | | | |
| | CUGCCUCCGUGCCCGAAUUCUACGGCAACA | | | | |
| | GCGACAAGUUCUACGGCUACGACCCCAGCA | | | | |
| | AAGACACCACAGAGAGCCCUUCCACCAUCU | | | | |
| | GGGUGUACACCCCUUCCCAGAAGGCCAUCG | | | | |
| | GCAGCAGAUUCGCUCAGGGCAGACCUAUG | | | | |
| | AAUAACGACGGCUCUUUCACCAUCACAAU | | | | |
| | GAAGGCCCCCCCCUUCGAGCAGGGCAAAGA | | | | |
| | CUUCGUGGUGCUGACAACCAAGGCCCACG | | | | |
| | GCGUGGGCAAGACAGAUCACUCCGACGAC | | | | |
| | ACCCGGACCCCUGUGACCUACAGAGAGGCA | | | | |
| | ACACCUGCUCCCACCGGGCCAAAGACCCCU | | | | |
| | AUCAGCGGAAGCAUCCUGGCCAUCUAUAG | | | | |
| | CACCGUGGCUAGCUCUCUGGUGCUGCUGG | | | | |
| | UGAGCCUGGGAGCUAUCAGCUUUGGGAGC | | | | |
| | GGCUGAUAA | | | | |
| 379 | AUGAAGGCCAAGCUGCUGGUGCUGCUGUG | Quad- | 386 | | |
| | UACCUUCACUGCAACAUAUGCUAUGGUGG | CAMP2- | | | |
| | AGCCCACAACCACAAUCUCUGCCACAAGCA | H4V3- | | | |
| | CCCACGAGCUGUCUGCCAGCGACGCCAGAA | f-PRO- | | | |
| | ACUCCAUCCAGCUGCUGAAUGCCCACAUCG | 028-V7- | | | |
| | CCACACUGCAGAGCGUGCAGAAAUCCGUG | F16_ssHA | | | |
| | CCUGGCAGCGAUUACAGCGACCAGAUCCG | codon | | | |
| | GGACCUGCUGAAGGCCGCCUUUGAUCUGA | optimization #1 | | | |
| | GAGGCCUGAUCGAAACCCUGGCCCACGGCG | | | | |
| | GCAUUCCCUUCUAUGACCCCUCCACCAUCA | | | | |
| | UGCCCAGAAUCAAGCUGGUGGCCACCACCA | | | | |
| | UCGACACCAUCCACACAGCUACAACCACCC | | | | |
| | UGCAGAACAAGGUGCGGCCAGCCCAUGUG | | | | |
| | GAACUGGGCCUGGAGGUGACAAAGGCCGU | | | | |
| | GCUGCUGACCGCCAAUCCUGCCAGCACAGC | | | | |
| | AAAAGAACUGGACGCCGAGGGAGCUGCUC | | | | |
| | UGAAGGCCAGACUGGAAAAGGUGAGCCAG | | | | |
| | UACCCCGACCUGACACCUAAUGAUGUGGCC | | | | |
| | ACAGUGUACGUGAGAACCAAUUUCAGCAA | | | | |
| | GACCAUCUGGCAGGUGAGAGCCAACAGAG | | | | |
| | AUCGGUACAUCCUGGGCCACAAGUCCGCCG | | | | |
| | CCGUGUACAAGACCCUGAAUCACGCCAUCA | | | | |
| | CCAAGGCCGUGGGCGUGCGGCUGAAUCCU | | | | |
| | AAGACCACCGUGGGCAACAUCCAGGCCGCC | | | | |
| | AGAACCGAACUGCUGGCCGCCUACCAGACC | | | | |
| | GCCUUCAACAGCCCCGAUGUGAAGAAGGC | | | | |
| | CGCUAGCUCCAAUAGACCCAGAUCCGUGGC | | | | |
| | CCAGGCCGCCAUCGCCACAGAUGGCAAGGG | | | | |
| | GAUCAUCGACAAGGAUUCCAGAGACGCUG | | | | |
| | UGAUCAACGACGCAAAGCUGAGAGCCGCC | | | | |
| | AUCGCCGGAGCUCUGGUGAAGGCCGGCUU | | | | |
| | UAGCAGCGCCGACGCCGUGGCACUGGCCCC | | | | |
| | CAGAAUUGCAAAGGAGAUGGCAAAGGAGG | | | | |
| | GCGUGCUGCUGAUCAAUCACCACAAGCUG | | | | |
| | AAGGCCCUGAUCGGCGCACAGCUGGGCCU | | | | |
| | GCUGACCGACGCAAAGAUUCAGAGAGCCG | | | | |
| | CCGCAGCCGUGGACCUGGGCAUCAAGGCCA | | | | |
| | CCCUGGCUGCCACCAUCAUCCCUAACGCCC | | | | |
| | UGGGCAGCGCCGCCUUCAAGAAUGCAGUG | | | | |
| | AUCGCCAACCUGGUGGCCGCCGGAAUCGAC | | | | |
| | AAGCACCUGGCCAGAGCCACAGCCGUCGCU | | | | |
| | AUCGUGGCCACAGCCCUGAAUCCCGCCCUG | | | | |
| | GGCCCCAUCGCCAAAUUUGAACUGAUCAA | | | | |
| | GGCAGAAAUCGCUGCCCAGGCCGCCCUGCU | | | | |
| | CAUCAGAAGAGGCGUCCAUCUGCAGAAGG | | | | |
| | CCGCCAUCGAGCACGUGAUUGGCAGAGCC | | | | |
| | UUCGACGCCGCCGUGGCCACAGCUAUCAUU | | | | |
| | UCUUCCCCUAUCCUGUCUGCCAGAAUCGUG | | | | |
| | ACACACCUGGUGAGAGCCGGAAUCGACAA | | | | |
| | GUCCAUCGCCAUCUCCCUGGCCCCCCACAU | | | | |
| | CGUGAAGAGACUGGCUAAGGAGCCUCUGC | | | | |
| | UGGCCUUCAAUACAGCCAAGCUGAUGAAG | | | | |
| | AACAUCACAAGACAGAUCGUGGACGUGAU | | | | |
| | CACCGCCGACAAGGCCAUCAAGACAGCCGA | | | | |
| | GCAGCUGGAGAAGGAACUGCCAGCCCUGG | | | | |
| | ACGACCUGGUGAAGAAGGCCAGCAGCCCU | | | | |
| | CCCAAGCCUACCCCUACCCCAACCCCUACA | | | | |
| | CCUACAGCCGGACCUACCGUGACCGUGAUC | | | | |
| | CCUGUGGGCAGAGAGGGCGGCGACAUCAC | | | | |
| | CAUCAGCGGCAAGGGCUUCAGCACAACAG | | | | |
| | GCUUUGGCGUGUACGUGGCCGUGGCCCCA | | | | |
| | GCCAGCGUGCCUGAGUUUUACGGCAAUAG | | | | |
| | CGACAAGUUCUACGGCUACGACCCCUCCAA | | | | |
| | GGACACCACCGAGAGCCCAAGCACCAUCUG | | | | |
| | GGUGUACACACCUAGCCAGAAGGCCAUCG | | | | |
| | GCAGCAGAUUUGCCCAGGGCAGGCCUAUG | | | | |
| | AAUAAUGACGGCAGCUUCACAAUCACAAU | | | | |
| | GAAGGCCCCCCCCUUUGAGCAGGGCAAGG | | | | |
| | ACUUCGUGGUGCUGACCACCAAGGCCCACG | | | | |
| | GCGUGGGCAAGACAGAUCACAGCGACGAC | | | | |
| | ACCCGGACCCCCGUGACCUACAGAGAAGCA | | | | |
| | ACACCUGCCCCUACCGGGCCUAAGACACCC | | | | |
| | AUCUGAUAA | | | | |
| 380 | AUGAAGGCUAAGCUGCUGGUGCUGCUGUG | Quad- | 387 | | 370 |
| | CACAUUCACAGCCACCUACGCCGUGGAGCC | CAMP2- | | | |
| | UACCACCACAAUCAGCGCCACCAGCACCCA | H4V3- | | | |
| | CGAGCUGAGCGCCAGCGAUGCCAGAAAUA | f-PRO- | | | |
| | GCAUCCAGCUGCUGAAUGCCCACAUCGCCA | 028-V7- | | | |
| | CCCUGCAGUCCGUGCAGAAAAGCGUGCCU | F16_ssHA | | | |
| | GGAAGCGAUUACAGCGAUCAGAUCAGGGA | codon | | | |
| | CCUGCUGAAGGCCGCCUUCGAUCUGAGAG | optimization #2 | | | |
| | GCCUGAUCGAGACACUGGCCCACGGCGGCA | | | | |
| | UCCCUUUCUACGACCCCAGCACCAUCAUGC | | | | |
| | CUAGAAUCAAACUGGUGGCCACCACCAUC | | | | |
| | GACACCAUCCACACCGCAACAACAACCCUG | | | | |
| | CAGAAUAAGGUGCGGCCUGCCCACGUGGA | | | | |
| | ACUGGGACUGGAAGUGACAAAGGCCGUGC | | | | |
| | UGCUGACCGCUAAUCCCGCCUCCACCGCCA | | | | |
| | AGGAGCUGGAUGCCGAAGGAGCUGCCCUG | | | | |
| | AAAGCCAGACUGGAGAAGGUGAGCCAGUA | | | | |
| | CCCUGAUCUGACCCCUAACGACGUGGCCAC | | | | |
| | CGUGUACGUGAGAACCAAUUUCAGCAAGA | | | | |
| | CCAUCUGGCAGGUGAGAGCCAAUCGGGAC | | | | |
| | AGAUACAUCCUGGGCCACAAGUCUGCCGCC | | | | |
| | GUGUAUAAAACCCUGAAUCACGCCAUCAC | | | | |
| | CAAGGCCGUGGGCGUGAGACUGAACCCUA | | | | |
| | AGACAACCGUGGGCAACAUCCAGGCCGCCA | | | | |
| | GAACAGAGCUGCUGGCCGCCUACCAGACA | | | | |
| | GCCUUCAAUAGCCCCGACGUGAAGAAGGC | | | | |
| | CGCUUCCAGCAAUAGACCCCGGAGCGUGGC | | | | |
| | ACAGGCUGCUAUUGCCACCGACGGAAAGG | | | | |
| | GCAUCAUCGAUAAAGACAGCCGGGAUGCC | | | | |
| | GUGAUCAAUGACGCCAAGCUGAGAGCAGC | | | | |
| | AAUCGCCGGCGCUCUGGUGAAGGCAGGCU | | | | |
| | UCAGCAGCGCCGACGCCGUGGCUCUGGCCC | | | | |
| | CUAGAAUCGCUAAGGAGAUGGCCAAGGAG | | | | |
| | GGCGUGCUGCUGAUCAAUCACCACAAGCU | | | | |
| | GAAGGCCCUGAUCGGCGCCCAGCUGGGAC | | | | |
| | UGCUGACAGAUGCCAAGAUCCAGAGAGCC | | | | |
| | GCUGCAGCCGUGGAUCUGGGCAUCAAGGC | | | | |
| | CACACUGGCCGCCACCAUCAUCCCAAACGC | | | | |
| | CCUGGGCAGCGCCGCCUUCAAAAAUGCCGU | | | | |
| | GAUUGCCAACCUGGUGGCCGCCGGCAUCG | | | | |
| | AUAAGCACCUGGCCAGAGCCACAGCCGUG | | | | |
| | GCCAUCGUGGCCACAGCCCUGAAUCCCGCA | | | | |
| | CUGGGACCUAUUGCCAAGUUCGAACUCAU | | | | |
| | UAAGGCCGAAAUCGCAGCCCAAGCCGCCCU | | | | |
| | GCUGAUUCGGCGGGGCGUGCACCUGCAGA | | | | |
| | AAGCCGCCAUUGAGCACGUGAUCGGCAGA | | | | |
| | GCCUUCGAUGCCGCUGUGGCCACCGCCAUC | | | | |
| | AUCUCCUCUCCUAUCCUGUCUGCUAGAAUC | | | | |
| | GUGACCCACCUGGUCAGAGCCGGAAUCGA | | | | |
| | CAAGUCUAUCGCUAUCAGCCUGGCCCCCCA | | | | |
| | CAUCGUGAAGAGACUGGCCAAAGAGCCCC | | | | |
| | UGCUGGCCUUCAACACAGCCAAGCUCAUG | | | | |
| | AAGAAUAUCACCAGACAGAUCGUGGACGU | | | | |
| | GAUCACCGCCGACAAGGCCAUCAAAACCGC | | | | |
| | UGAGCAGCUGGAGAAGGAGCUGCCCGCCC | | | | |
| | UGGAUGACCUGGUGAAGAAGGCCAGCAGC | | | | |
| | CCCCCCAAGCCUACACCUACACCUACCCCA | | | | |
| | ACCCCUACAGCCGGCCCUACAGUGACCGUG | | | | |
| | AUUCCUGUGGGCAGAGAAGGAGGCGACAU | | | | |
| | CACCAUCAGCGGAAAGGGCUUCUCUACCAC | | | | |
| | AGGCUUCGGCGUGUACGUGGCCGUGGCCC | | | | |
| | CUGCCUCCGUGCCCGAAUUCUACGGCAACA | | | | |
| | GCGACAAGUUCUACGGCUACGACCCCAGCA | | | | |
| | AAGACACCACAGAGAGCCCUUCCACCAUCU | | | | |
| | GGGUGUACACCCCUUCCCAGAAGGCCAUCG | | | | |
| | GCAGCAGAUUCGCUCAGGGCAGACCUAUG | | | | |
| | AAUAACGACGGCUCUUUCACCAUCACAAU | | | | |
| | GAAGGCCCCCCCCUUCGAGCAGGGCAAAGA | | | | |
| | CUUCGUGGUGCUGACAACCAAGGCCCACG | | | | |
| | GCGUGGGCAAGACAGAUCACUCCGACGAC | | | | |
| | ACCCGGACCCCUGUGACCUACAGAGAGGCA | | | | |
| | ACACCUGCUCCCACCGGGCCAAAGACCCCU | | | | |
| | AUCUGAUAA | | | | |
| 381 | AUGAAGGCCAAGCUGCUGGUGCUGCUGUG | Quad- | 388 | | 371 |
| | UACCUUCACUGCAACAUAUGCUGUGGAGC | CAMP2- | | | |
| | CCACAACCACAAUCUCUGCCACAAGCACCC | Nter- | | | |
| | ACGAGCUGUCUGCCAGCGACGCCAGAAAC | link- | | | |
| | UCCAUCCAGCUGCUGAAUGCCCACAUCGCC | H4V3-f- | | | |
| | ACACUGCAGAGCGUGCAGAAAUCCGUGCC | PRO- | | | |
| | UGGCAGCGAUUACAGCGACCAGAUCCGGG | 028-V7- | | | |
| | ACCUGCUGAAGGCCGCCUUUGAUCUGAGA | F16_ssHA | | | |
| | GGCCUGAUCGAAACCCUGGCCCACGGCGGC | codon | | | |
| | AUUCCCUUCUAUGACCCCUCCACCAUCAUG | optimization #1 | | | |
| | CCCAGAAUCAAGCUGGUGGCCACCACCAUC | | | | |
| | GACACCAUCCACACAGCUACAACCACCCUG | | | | |
| | CAGAACAAGGUGCGGCCAGCCCAUGUGGA | | | | |
| | ACUGGGCCUGGAGGUGACAAAGGCCGUGC | | | | |
| | UGCUGACCGCCAAUCCUGCCAGCACAGCAA | | | | |
| | AAGAACUGGACGCCGAGGGAGCUGCUCUG | | | | |
| | AAGGCCAGACUGGAAAAGGUGAGCCAGUA | | | | |
| | CCCCGACCUGACACCUAAUGAUGUGGCCAC | | | | |
| | AAGCUCCAAUAGACCCAGAUCCGUGGCCCA | | | | |
| | GGCCGCCAUCGCCACAGAUGGCAAGGGGA | | | | |
| | UCAUCGACAAGGAUUCCAGAGACGCUGUG | | | | |
| | AUCAACGACGCAAAGCUGAGAGCCGCCAU | | | | |
| | CGCCGGAGCUCUGGUGAAGGCCGGCUUUA | | | | |
| | GCAGCGCCGACGCCGUGGCACUGGCCCCCA | | | | |
| | GAAUUGCAAAGGAGAUGGCAAAGGAGGGC | | | | |
| | GUGCUGCUGAUCAAUCACCACAAGCUGAA | | | | |
| | GGCCCUGAUCGGCGCACAGCUGGGCCUGCU | | | | |
| | GACCGACGCAAAGAUUCAGAGAGCCGCCG | | | | |
| | CAGCCGUGGACCUGGGCAUCAAGGCCACCC | | | | |
| | UGGCUGCCACCAUCAUCCCUAACGCCCUGG | | | | |
| | GCAGCGCCGCCUUCAAGAAUGCAGUGAUC | | | | |
| | GCCAACCUGGUGGCCGCCGGAAUCGACAA | | | | |
| | GCACCUGGCCAGAGCCACAGCCGUCGCUAU | | | | |
| | CGUGGCCACAGCCCUGAAUCCCGCCCUGGG | | | | |
| | CCCCAUCGCCAAAUUUGAACUGAUCAAGG | | | | |
| | CAGAAAUCGCUGCCCAGGCCGCCCUGCUCA | | | | |
| | UCAGAAGAGGCGUCCAUCUGCAGAAGGCC | | | | |
| | GCCAUCGAGCACGUGAUUGGCAGAGCCUU | | | | |
| | CGACGCCGCCGUGGCCACAGCUAUCAUUUC | | | | |
| | UUCCCCUAUCCUGUCUGCCAGAAUCGUGAC | | | | |
| | ACACCUGGUGAGAGCCGGAAUCGACAAGU | | | | |
| | CCAUCGCCAUCUCCCUGGCCCCCCACAUCG | | | | |
| | UGAAGAGACUGGCUAAGGAGCCUCUGCUG | | | | |
| | GCCUUCAAUACAGCCAAGCUGAUGAAGAA | | | | |
| | CAUCACAAGACAGAUCGUGGACGUGAUCA | | | | |
| | CCGCCGACAAGGCCAUCAAGACAGCCGAGC | | | | |
| | AGCUGGAGAAGGAACUGCCAGCCCUGGAC | | | | |
| | GACCUGGUGAAGAAGGCCAGCAGCCCUCCC | | | | |
| | AAGCCUACCCCUACCCCAACCCCUACACCU | | | | |
| | ACAGCCGGACCUACCGUGACCGUGAUCCCU | | | | |
| | GUGGGCAGAGAGGGCGGCGACAUCACCAU | | | | |
| | CAGCGGCAAGGGCUUCAGCACAACAGGCU | | | | |
| | UUGGCGUGUACGUGGCCGUGGCCCCAGCC | | | | |
| | AGCGUGCCUGAGUUUUACGGCAAUAGCGA | | | | |
| | CAAGUUCUACGGCUACGACCCCUCCAAGGA | | | | |
| | CACCACCGAGAGCCCAAGCACCAUCUGGGU | | | | |
| | GUACACACCUAGCCAGAAGGCCAUCGGCA | | | | |
| | GCAGAUUUGCCCAGGGCAGGCCUAUGAAU | | | | |
| | AAUGACGGCAGCUUCACAAUCACAAUGAA | | | | |
| | GGCCCCCCCCUUUGAGCAGGGCAAGGACUU | | | | |
| | CGUGGUGCUGACCACCAAGGCCCACGGCGU | | | | |
| | GGGCAAGACAGAUCACAGCGACGACACCC | | | | |
| | GGACCCCCGUGACCUACAGAGAAGCAACAC | | | | |
| | CUGCCCCUACCGGGCCUAAGACACCCAUCU | | | | |
| | GAUAA | | | | |
| 382 | AUGAAGGCUAAGCUGCUGGUGCUGCUGUG | Quad- | 389 | | 371 |
| | CACAUUCACAGCCACCUACGCCGUGGAGCC | CAMP2- | | | |
| | UACCACCACAAUCAGCGCCACCAGCACCCA | Nter- | | | |
| | CGAGCUGAGCGCCAGCGAUGCCAGAAAUA | link- | | | |
| | GCAUCCAGCUGCUGAAUGCCCACAUCGCCA | H4V3-f- | | | |
| | CCCUGCAGUCCGUGCAGAAAAGCGUGCCU | PRO- | | | |
| | GGAAGCGAUUACAGCGAUCAGAUCAGGGA | 028-V7- | | | |
| | CCUGCUGAAGGCCGCCUUCGAUCUGAGAG | F16_ssHA | | | |
| | GCCUGAUCGAGACACUGGCCCACGGCGGCA | codon | | | |
| | UCCCUUUCUACGACCCCAGCACCAUCAUGC | optimization #2 | | | |
| | CUAGAAUCAAACUGGUGGCCACCACCAUC | | | | |
| | GACACCAUCCACACCGCAACAACAACCCUG | | | | |
| | CAGAAUAAGGUGCGGCCUGCCCACGUGGA | | | | |
| | ACUGGGACUGGAAGUGACAAAGGCCGUGC | | | | |
| | UGCUGACCGCUAAUCCCGCCUCCACCGCCA | | | | |
| | AGGAGCUGGAUGCCGAAGGAGCUGCCCUG | | | | |
| | AAAGCCAGACUGGAGAAGGUGAGCCAGUA | | | | |
| | CCCUGAUCUGACCCCUAACGACGUGGCCAC | | | | |
| | CUCCAGCAAUAGACCCCGGAGCGUGGCACA | | | | |
| | GGCUGCUAUUGCCACCGACGGAAAGGGCA | | | | |
| | UCAUCGAUAAAGACAGCCGGGAUGCCGUG | | | | |
| | AUCAAUGACGCCAAGCUGAGAGCAGCAAU | | | | |
| | CGCCGGCGCUCUGGUGAAGGCAGGCUUCA | | | | |
| | GCAGCGCCGACGCCGUGGCUCUGGCCCCUA | | | | |
| | GAAUCGCUAAGGAGAUGGCCAAGGAGGGC | | | | |
| | GUGCUGCUGAUCAAUCACCACAAGCUGAA | | | | |
| | GGCCCUGAUCGGCGCCCAGCUGGGACUGCU | | | | |
| | GACAGAUGCCAAGAUCCAGAGAGCCGCUG | | | | |
| | CAGCCGUGGAUCUGGGCAUCAAGGCCACA | | | | |
| | CUGGCCGCCACCAUCAUCCCAAACGCCCUG | | | | |
| | GGCAGCGCCGCCUUCAAAAAUGCCGUGAU | | | | |
| | UGCCAACCUGGUGGCCGCCGGCAUCGAUA | | | | |
| | AGCACCUGGCCAGAGCCACAGCCGUGGCCA | | | | |
| | UCGUGGCCACAGCCCUGAAUCCCGCACUGG | | | | |
| | GACCUAUUGCCAAGUUCGAACUCAUUAAG | | | | |
| | GCCGAAAUCGCAGCCCAAGCCGCCCUGCUG | | | | |
| | AUUCGGCGGGGCGUGCACCUGCAGAAAGC | | | | |
| | CGCCAUUGAGCACGUGAUCGGCAGAGCCU | | | | |
| | UCGAUGCCGCUGUGGCCACCGCCAUCAUCU | | | | |
| | CCUCUCCUAUCCUGUCUGCUAGAAUCGUG | | | | |
| | ACCCACCUGGUCAGAGCCGGAAUCGACAA | | | | |
| | GUCUAUCGCUAUCAGCCUGGCCCCCCACAU | | | | |
| | CGUGAAGAGACUGGCCAAAGAGCCCCUGC | | | | |
| | UGGCCUUCAACACAGCCAAGCUCAUGAAG | | | | |
| | AAUAUCACCAGACAGAUCGUGGACGUGAU | | | | |
| | CACCGCCGACAAGGCCAUCAAAACCGCUGA | | | | |
| | GCAGCUGGAGAAGGAGCUGCCCGCCCUGG | | | | |
| | AUGACCUGGUGAAGAAGGCCAGCAGCCCC | | | | |
| | CCCAAGCCUACACCUACACCUACCCCAACC | | | | |
| | CCUACAGCCGGCCCUACAGUGACCGUGAUU | | | | |
| | CCUGUGGGCAGAGAAGGAGGCGACAUCAC | | | | |
| | CAUCAGCGGAAAGGGCUUCUCUACCACAG | | | | |
| | GCUUCGGCGUGUACGUGGCCGUGGCCCCU | | | | |
| | GCCUCCGUGCCCGAAUUCUACGGCAACAGC | | | | |
| | GACAAGUUCUACGGCUACGACCCCAGCAA | | | | |
| | AGACACCACAGAGAGCCCUUCCACCAUCUG | | | | |
| | GGUGUACACCCCUUCCCAGAAGGCCAUCGG | | | | |
| | CAGCAGAUUCGCUCAGGGCAGACCUAUGA | | | | |
| | AUAACGACGGCUCUUUCACCAUCACAAUG | | | | |
| | AAGGCCCCCCCCUUCGAGCAGGGCAAAGAC | | | | |
| | UUCGUGGUGCUGACAACCAAGGCCCACGG | | | | |
| | CGUGGGCAAGACAGAUCACUCCGACGACA | | | | |
| | CCCGGACCCCUGUGACCUACAGAGAGGCAA | | | | |
| | CACCUGCUCCCACCGGGCCAAAGACCCCUA | | | | |
| | UCUGAUAA | | | | |
| 383 | AUGAAGGCUAAGCUGCUGGUGCUGCUGUG | Quad- | 390 | | 372 |
| | CACAUUCACAGCCACCUACGCCUCCAGCAA | H4V3-f- | | | |
| | UAGACCCCGGAGCGUGGCACAGGCUGCUA | PRO- | | | |
| | UUGCCACCGACGGAAAGGGCAUCAUCGAU | 028-V7- | | | |
| | AAAGACAGCCGGGAUGCCGUGAUCAAUGA | F16- | | | |
| | CGCCAAGCUGAGAGCAGCAAUCGCCGGCGC | nolink- | | | |
| | UCUGGUGAAGGCAGGCUUCAGCAGCGCCG | Cter- | | | |
| | ACGCCGUGGCUCUGGCCCCUAGAAUCGCUA | CAMP2 | | | |
| | AGGAGAUGGCCAAGGAGGGCGUGCUGCUG | ssHA | | | |
| | AUCAAUCACCACAAGCUGAAGGCCCUGAU | codon | | | |
| | CGGCGCCCAGCUGGGACUGCUGACAGAUG | optimization | | | |
| | CCAAGAUCCAGAGAGCCGCUGCAGCCGUG | #2 | | | |
| | GAUCUGGGCAUCAAGGCCACACUGGCCGCC | | | | |
| | ACCAUCAUCCCAAACGCCCUGGGCAGCGCC | | | | |
| | GCCUUCAAAAAUGCCGUGAUUGCCAACCU | | | | |
| | GGUGGCCGCCGGCAUCGAUAAGCACCUGG | | | | |
| | CCAGAGCCACAGCCGUGGCCAUCGUGGCCA | | | | |
| | CAGCCCUGAAUCCCGCACUGGGACCUAUUG | | | | |
| | CCAAGUUCGAACUCAUUAAGGCCGAAAUC | | | | |
| | GCAGCCCAAGCCGCCCUGCUGAUUCGGCGG | | | | |
| | GGCGUGCACCUGCAGAAAGCCGCCAUUGA | | | | |
| | GCACGUGAUCGGCAGAGCCUUCGAUGCCG | | | | |
| | CUGUGGCCACCGCCAUCAUCUCCUCUCCUA | | | | |
| | UCCUGUCUGCUAGAAUCGUGACCCACCUG | | | | |
| | GUCAGAGCCGGAAUCGACAAGUCUAUCGC | | | | |
| | UAUCAGCCUGGCCCCCCACAUCGUGAAGAG | | | | |
| | ACUGGCCAAAGAGCCCCUGCUGGCCUUCAA | | | | |
| | CACAGCCAAGCUCAUGAAGAAUAUCACCA | | | | |
| | GACAGAUCGUGGACGUGAUCACCGCCGAC | | | | |
| | AAGGCCAUCAAAACCGCUGAGCAGCUGGA | | | | |
| | GAAGGAGCUGCCCGCCCUGGAUGACCUGG | | | | |
| | UGAAGAAGGCCAGCAGCCCCCCCAAGCCUA | | | | |
| | CACCUACACCUACCCCAACCCCUACAGCCG | | | | |
| | GCCCUACAGUGACCGUGAUUCCUGUGGGC | | | | |
| | AGAGAAGGAGGCGACAUCACCAUCAGCGG | | | | |
| | AAAGGGCUUCUCUACCACAGGCUUCGGCG | | | | |
| | UGUACGUGGCCGUGGCCCCUGCCUCCGUGC | | | | |
| | CCGAAUUCUACGGCAACAGCGACAAGUUC | | | | |
| | UACGGCUACGACCCCAGCAAAGACACCACA | | | | |
| | GAGAGCCCUUCCACCAUCUGGGUGUACACC | | | | |
| | CCUUCCCAGAAGGCCAUCGGCAGCAGAUUC | | | | |
| | GCUCAGGGCAGACCUAUGAAUAACGACGG | | | | |
| | CUCUUUCACCAUCACAAUGAAGGCCCCCCC | | | | |
| | CUUCGAGCAGGGCAAAGACUUCGUGGUGC | | | | |
| | UGACAACCAAGGCCCACGGCGUGGGCAAG | | | | |
| | ACAGAUCACUCCGACGACACCCGGACCCCU | | | | |
| | GUGACCUACAGAGAGGCAACACCUGCUCCC | | | | |
| | ACCGGGCCAAAGACCCCUAUCGUGUACGU | | | | |
| | GAGAACCAAUUUCAGCAAGACCAUCUGGC | | | | |
| | AGGUGAGAGCCAAUCGGGACAGAUACAUC | | | | |
| | CUGGGCCACAAGUCUGCCGCCGUGUAUAA | | | | |
| | AACCCUGAAUCACGCCAUCACCAAGGCCGU | | | | |
| | GGGCGUGAGACUGAACCCUAAGACAACCG | | | | |
| | UGGGCAACAUCCAGGCCGCCAGAACAGAG | | | | |
| | CUGCUGGCCGCCUACCAGACAGCCUUCAAU | | | | |
| | AGCCCCGACGUGAAGAAGGCCGCUUGAUA | | | | |
| | A | | | | |
| 394 | AUGAAGGCCAAGCUGCUGGUGCUGCUGUG | Quad- | 393 | | 372 |
| | UACCUUCACUGCAACAUAUGCUAGCUCCA | H4V3-f- | | | |
| | AUAGACCCAGAUCCGUGGCCCAGGCCGCCA | PRO- | | | |
| | UCGCCACAGAUGGCAAGGGGAUCAUCGAC | 028-V7- | | | |
| | AAGGAUUCCAGAGACGCUGUGAUCAACGA | F16- | | | |
| | CGCAAAGCUGAGAGCCGCCAUCGCCGGAGC | nolink- | | | |
| | UCUGGUGAAGGCCGGCUUUAGCAGCGCCG | Cter- | | | |
| | ACGCCGUGGCACUGGCCCCCAGAAUUGCAA | CAMP2_ | | | |
| | AGGAGAUGGCAAAGGAGGGCGUGCUGCUG | SSHA | | | |
| | AUCAAUCACCACAAGCUGAAGGCCCUGAU | codon | | | |
| | CGGCGCACAGCUGGGCCUGCUGACCGACGC | optimization | | | |
| | AAAGAUUCAGAGAGCCGCCGCAGCCGUGG | #1 | | | |
| | ACCUGGGCAUCAAGGCCACCCUGGCUGCCA | | | | |
| | CCAUCAUCCCUAACGCCCUGGGCAGCGCCG | | | | |
| | CCUUCAAGAAUGCAGUGAUCGCCAACCUG | | | | |
| | GUGGCCGCCGGAAUCGACAAGCACCUGGCC | | | | |
| | AGAGCCACAGCCGUCGCUAUCGUGGCCACA | | | | |
| | GCCCUGAAUCCCGCCCUGGGCCCCAUCGCC | | | | |
| | AAAUUUGAACUGAUCAAGGCAGAAAUCGC | | | | |
| | UGCCCAGGCCGCCCUGCUCAUCAGAAGAGG | | | | |
| | CGUCCAUCUGCAGAAGGCCGCCAUCGAGCA | | | | |
| | CGUGAUUGGCAGAGCCUUCGACGCCGCCG | | | | |
| | UGGCCACAGCUAUCAUUUCUUCCCCUAUCC | | | | |
| | UGUCUGCCAGAAUCGUGACACACCUGGUG | | | | |
| | AGAGCCGGAAUCGACAAGUCCAUCGCCAU | | | | |
| | CUCCCUGGCCCCCCACAUCGUGAAGAGACU | | | | |
| | GGCUAAGGAGCCUCUGCUGGCCUUCAAUA | | | | |
| | CAGCCAAGCUGAUGAAGAACAUCACAAGA | | | | |
| | CAGAUCGUGGACGUGAUCACCGCCGACAA | | | | |
| | GGCCAUCAAGACAGCCGAGCAGCUGGAGA | | | | |
| | AGGAACUGCCAGCCCUGGACGACCUGGUG | | | | |
| | AAGAAGGCCAGCAGCCCUCCCAAGCCUACC | | | | |
| | CCUACCCCAACCCCUACACCUACAGCCGGA | | | | |
| | CCUACCGUGACCGUGAUCCCUGUGGGCAG | | | | |
| | AGAGGGCGGCGACAUCACCAUCAGCGGCA | | | | |
| | AGGGCUUCAGCACAACAGGCUUUGGCGUG | | | | |
| | UACGUGGCCGUGGCCCCAGCCAGCGUGCCU | | | | |
| | GAGUUUUACGGCAAUAGCGACAAGUUCUA | | | | |
| | CGGCUACGACCCCUCCAAGGACACCACCGA | | | | |
| | GAGCCCAAGCACCAUCUGGGUGUACACACC | | | | |
| | UAGCCAGAAGGCCAUCGGCAGCAGAUUUG | | | | |
| | CCCAGGGCAGGCCUAUGAAUAAUGACGGC | | | | |
| | AGCUUCACAAUCACAAUGAAGGCCCCCCCC | | | | |
| | UUUGAGCAGGGCAAGGACUUCGUGGUGCU | | | | |
| | GACCACCAAGGCCCACGGCGUGGGCAAGAC | | | | |
| | AGAUCACAGCGACGACACCCGGACCCCCGU | | | | |
| | GACCUACAGAGAAGCAACACCUGCCCCUAC | | | | |
| | CGGGCCUAAGACACCCAUCGUGUACGUGA | | | | |
| | GAACCAAUUUCAGCAAGACCAUCUGGCAG | | | | |
| | GUGAGAGCCAACAGAGAUCGGUACAUCCU | | | | |
| | GGGCCACAAGUCCGCCGCCGUGUACAAGAC | | | | |
| | CCUGAAUCACGCCAUCACCAAGGCCGUGGG | | | | |
| | CGUGCGGCUGAAUCCUAAGACCACCGUGG | | | | |
| | GCAACAUCCAGGCCGCCAGAACCGAACUGC | | | | |
| | UGGCCGCCUACCAGACCGCCUUCAACAGCC | | | | |
| | CCGAUGUGAAGAAGGCCGCUUGAUAA | | | | |
| 391 | AUGAAGGCCAAGCUGCUGGUGCUGCUGUG | CAMP2_ | | | |
| | CACCUUCACCGCCACCUACGCUGUGGAGCC | WT_HA | | | |
| | CACCACCACCAUCAGCGCAACCUCCACCCA | TMB | | | |
| | CGAACUGUCCGCCUCCGAUGCUAGGAACUC | | | | |
| | AAUCCAGCUGCUGAACGCCCACAUCGCCAC | | | | |
| | ACUGCAGAGCGUCCAGAAAUCAGUGCCCG | | | | |
| | GCAGCGACUACUCCGACCAGAUCCGGGAUC | | | | |
| | UCCUGAAGGCUGCAUUCGACCUGCGCGGCC | | | | |
| | UCAUCGAAACACUGGCCCACGGAGGCAUCC | | | | |
| | CCUUCUAUGACCCAUCCACCAUCAUGCCCC | | | | |
| | GGAUCAAGCUGGUGGCCACCACAAUCGAC | | | | |
| | ACCAUCCACACCGCCACCACCACCCUGCAG | | | | |
| | AACAAGGUGAGGCCCGCACACGUGGAGCU | | | | |
| | GGGGCUGGAGGUGACUAAGGCCGUGCUGC | | | | |
| | UGACAGCAAACCCCGCCUCAACCGCGAAGG | | | | |
| | AGUUGGAUGCCGAGGGCGCCGCACUGAAG | | | | |
| | GCCCGCCUGGAGAAGGUGAGCCAGUAUCC | | | | |
| | CGAUCUGACCCCCAACGACGUGGCCACCGU | | | | |
| | GUAUGUGAGGACCAAUUUCUCCAAGACCA | | | | |
| | UCUGGCAGGUCAGAGCCAACAGAGACAGG | | | | |
| | UAUAUCCUGGGCCACAAGAGCGCUGCCGU | | | | |
| | GUACAAGACCCUGAACCACGCCAUCACCAA | | | | |
| | GGCCGUGGGGGUGCGGCUGAACCCUAAGA | | | | |
| | CCACCGUGGGGAACAUCCAGGCUGCCAGA | | | | |
| | ACCGAGCUGCUGGCCGCCUAUCAGACCGCA | | | | |
| | UUCAAUUCCCCCGAUGUGAAGAAGGCCGC | | | | |
| | CAGCGGCUCCAUCCUGGCCAUCUACUCCAC | | | | |
| | CGUGGCUAGUAGCCUGGUGCUGCUGGUGU | | | | |
| | CCCUGGGAGCCAUCAGCUUCGGGAGCGGC | | | | |
| | UAA | | | | |
| 392 | AUGAAAGCCAAGCUGCUCGUGCUGCUGUG | CAMP2_ | | | |
| | UACCUUCACCGCCACCUACGCCGUGGAACC | WT_HA TMB | | | |
| | CACCACCACAAUCUCCGCCACCUCCACCCA | | | | |
| | CGAGCUGAGCGCCAGCGACGCCAGAAACA | | | | |
| | GCAUUCAGCUGCUGAAUGCCCACAUCGCCA | | | | |
| | CCCUGCAGAGCGUGCAGAAGUCCGUGCCU | | | | |
| | GGCAGCGACUACAGCGACCAGAUCCGGGA | | | | |
| | CCUGCUGAAGGCCGCCUUCGACCUGAGAG | | | | |
| | GCCUGAUUGAGACACUGGCCCACGGCGGC | | | | |
| | AUUCCUUUCUACGAUCCUUCUACCAUCAU | | | | |
| | GCCUAGAAUCAAGCUGGUGGCCACCACCA | | | | |
| | UCGAUACCAUCCACACAGCCACCACCACAC | | | | |
| | UGCAGAACAAGGUGCGGCCUGCUCACGUG | | | | |
| | GAACUGGGCCUGGAAGUGACCAAGGCCGU | | | | |
| | GCUGCUGACCGCCAAUCCUGCCUCCACCGC | | | | |
| | CAAGGAGCUGGACGCAGAAGGCGCUGCCC | | | | |
| | UGAAGGCCAGACUGGAGAAGGUGAGCCAG | | | | |
| | UAUCCCGACCUGACCCCAAACGAUGUGGCC | | | | |
| | ACCGUGUACGUGCGGACAAACUUCAGCAA | | | | |
| | GACCAUCUGGCAGGUGAGAGCCAACCGGG | | | | |
| | ACAGAUACAUCCUGGGCCACAAGAGCGCC | | | | |
| | GCCGUGUAUAAGACCCUGAACCACGCCAUC | | | | |
| | ACAAAGGCCGUGGGCGUGAGACUGAACCC | | | | |
| | CAAGACCACAGUGGGCAACAUCCAGGCCGC | | | | |
| | CAGGACCGAACUGCUGGCCGCCUACCAGAC | | | | |
| | CGCCUUCAAUUCCCCUGACGUGAAGAAGG | | | | |
| | CCGCCUCUGGCAGCAUCCUGGCCAUCUACA | | | | |
| | GCACCGUGGCCAGCUCUCUGGUGCUGCUG | | | | |
| | GUGUCUCUGGGCGCCAUCAGCUUCGGCAG | | | | |
| | CGGCUGA | | | | |

TABLE 8

| | | Encodes |
|---|---|---|
| | | polypeptide |
| | | sequence |
| SEQ ID | | according to |
| NO | Nucleotide sequence | SEQ ID NO |
| 179 | UCAUCCAACAGGCCUCGCUCCGUGGCCCAGGCUGCUAUUGCUACCGACGGC | 70 |
| | AAGGGAAUCAUCGAUAAAGACUCCAGGGAUGCCGUGAUCAACGAUGCCAA | |
| | GCUCCGGGCCGCAAUUGCCGGAGCCCUGGUGAAAGCCGGGUUCAGCUCCG | |
| | CCGAUGCUGUGGCUCUGGCUCCAAGGAUUGCUAAGGAGAUGGCCAAAGAG | |
| | GGGGUGCUGCUGAUUAACCACCACAAGCUGAAGGCACUGAUCGGAGCACA | |
| | GCUGGGACUGCUGACUGACGCCAAAAUCCAGCGCGCCGCUGCAGCAGUCG | |
| | ACCUGGGCAUCAAGGCCACACUGGCUGCUACCAUCAUUCCUAACGCUCUG | |
| | GGCAGCGCAGCCUUUAAGAAUGCCGUUAUCGCCAAUCUCGUGGCUGCUGG | |
| | AAUUGAUAAGCACCUGGCCAGAGCCACAGCUGUGGCCAUCGUGGCCACUG | |
| | CCCUGAACCCAGCAUUGGGCCCUAUCGCUAAGUUUGAGCUGAUUAAGGCC | |
| | GAGAUCGCCGCUCAGGCUGCCCUGCUGAUUCGCAGGGGAGUGCACCUGCA | |
| | GAAGGCUGCCAUCGAGCACGUCAUCGGCAGAGCAUUCGACGCCGCUGUCG | |
| | CCACUGCUAUCAUCUCUAGCCCAAUCCUGAGCGCCAGGAUCGUCACUCACC | |
| | UGGUGAGAGCUGGGAUUGACAAAUCCAUCGCUAUCAGCCUGGCCCCUCAC | |
| | AUCGUGAAGAGGCUGGCUAAGGAACCACUGCUGGCUUUUAACACUGCUAA | |
| | GCUGAUGAAGAAUAUUACAAGACAGAUCGUGGACGUCAUCACCGCCGACA | |
| | AAGCCAUCAAGACAGCCGAGCAGCUCGAGAAGGAGCUGCCUGCCCUGGAU | |
| | GACCUGGUGAAGAAGGCAUCCUCCCCCCCUAAGCCAACCCCUACCCCAACC | |
| | CCUACACCUACUUAA | |
| 181 | UCAUCCAACAGGCCUCGCUCCGUGGCCCAGGCUGCUAUUGCUACCGACGG | 72 |
| | CAAGGGAAUCAUCGAUAAAGACUCCAGGGAUGCCGUGAUCAACGAUGCCA | |
| | AGCUCCGGGCCGCAAUUGCCGGAGCCCUGGUGAAAGCCGGGUUCAGCUCC | |
| | GCCGAUGCUGUGGCUCUGGCUCCAAGGAUUGCUAAGGAGAUGGCCAAAGA | |
| | GGGGGUGCUGCUGAUUAACCACCACAAGCUGAAGGCACUGAUCGGAGCAC | |
| | AGCUGGGACUGCUGACUGACGCCAAAAUCCAGCGCGCCGCUGCAGCAGUC | |
| | GACCUGGGCAUCAAGGCCACACUGGCUGCUACCAUCAUUCCUAACGCUCU | |
| | GGGCAGCGCAGCCUUUAAGAAUGCCGUUAUCGCCAAUCUCGUGGCUGCUG | |
| | GAAUUGAUAAGCACCUGGCCAGAGCCACAGCUGUGGCCAUCGUGGCCACU | |
| | GCCCUGAACCCAGCAUUGGGCCCUAUCGCUAAGUUUGAGCUGAUUAAGGC | |
| | CGAGAUCGCCGCUCAGGCUGCCCUGCUGAUUCGCAGGGGAGUGCACCUGC | |
| | AGAAGGCUGCCAUCGAGCACGUCAUCGGCAGAGCAUUCGACGCCGCUGUC | |
| | GCCACUGCUAUCAUCUCUAGCCCAAUCCUGAGCGCCAGGAUCGUCACUCAC | |
| | CUGGUGAGAGCUGGGAUUGACAAAUCCAUCGCUAUCAGCCUGGCCCCUCA | |
| | CAUCGUGAAGAGGCUGGCUAAGGAACCACUGCUGGCUUUUAACACUGCUA | |
| | AGCUGAUGAAGCAGAUUACAAGACAGAUCGUGGACGUCAUCACCGCCGAC | |
| | AAAGCCAUCAAGACAGCCGAGCAGCUCGAGAAGGAGCUGCCUGCCCUGGA | |
| | UGACCUGGUGAAGAAGGCAUCCUCCCCCCCUAAGCCAACCCCUACCCCAAC | |
| | CCCUACACCUACUUAA | |
| 182 | GCAGGCCCUACCGUCACAGUGAUCCCUGUGGGCAGAGAAGGUGGCGACAU | 73 |
| | CACCAUCAGCGGCAAGGGCUUCAGCACCACAGGCUUUGGCGUGUACGUGG | |
| | CCGUGGCACCUGCUAGCGUGCCCGAAUUCUACGGCAAUAGCGACAAGUUC | |
| | UACGGCUACGACCCCAGCAAGGACACCACCGAGAGCCCUAGCACCAUCUGG | |
| | GUGUACACCCCUAGCCAGAAGGCCAUCGGCUCUCGGUUCGCCCAGGGCAG | |
| | ACCCAUGAACAAUGACGGCAGCUUCACAAUCACCAUGAAGGCCCCCCCUUU | |
| | CGAGCAGGGCAAAGACUUCGUGGUGCUGACCACAAAGGCCCAUGGCGUGG | |
| | GCAAAACCGAUCACAGCGACGACACACGGACACCUGUCACCUACAGAGAG | |
| | GCUACCCCUGCCCCUACAGGCCCUAAGACACCCAUCGCCCCUAGCAAGCAG | |
| | CCUAGCAAGCAGGCCGCCCCUAGCAAGCAGGUGAAGCCUUCUAAGCAGGC | |
| | CGGCCCCAACAAGCAGUCCACCACACCUCAGCAGAAGACAGCCGAGCACAG | |
| | AAGCCAGACCCCUGCCGCCCACCGGACCAUGACAAAGCAGGUGAGCACCAU | |
| | CGGCGCCAGCAAGGUGACAAGCGGCUCUCUGACCUGGGGCAUCAGAACCU | |
| | CUUUUACCUCCUACCUGAGAGGCCCUAUCGCCAACGGCUCUUGGAAGCUG | |
| | AGCGGAGGCGCCAAUUGGAAUGGCUCCGCCUUCACCUUCCCUCUGACCAGC | |
| | GGCAGCUUCGACCCAGCCACCAAGUCCGGAAGCCUGAAGUACAGCGGCAG | |
| | CGUGCACAUGACAGGCCACCACGGCAUCCUGGACAUGACCCUGGCCGAGCC | |
| | AAGCCUGCAGAUCAAGGGAAGCACCGGCCACCUGUAUCUGGACGUGAAAA | |
| | GCAGCAGCAUGGACGGCAAGAAGACCAAUUACGGCAGAGUGGACUUCGCC | |
| | ACCUUCGGAGUGAGCGUGUCUGGCAACGCCGCCAUCAAGGGCAGCCCUGU | |
| | GAAGCUGACCGCCACAGGCGCUAAGGCUUUUGCCGGCUUCUACAGAGCCG | |
| | GCGAGCCUAUGAACCCUCUGAGCACCAAUCUGACCCUGAGCGCCGAGAAG | |
| | GUGUCCCACAACGUGACCGUGGACGCAGUGACAGGCAAGGUGAUCGGCGA | |
| | CGACUCUGGCAAGGGAGCCGGCCGGGGCCUGCCCGUGACAUAA | |
| 189 | UCCUCUAAUAGACCAAGAUCCGUGGCUCAGGCCGCCAUCGCUACAGAUGG | 80 |
| | GAAAGGAAUCAUCGAUAAGGAUAGUCGGGAUGCAGUGAUCAACGACGCCA | |
| | AACUCCGAGCUGCCAUCGCCGGCGCCCUGGUGAAGGCUGGGUUUUCCAGC | |
| | GCCGACGCAGUGGCCCUGGCACCUAGAAUCGCCAAGGAGAUGGCAAAGGA | |
| | GGGCGUCCUGCUGAUUAAUCACCACAAGCUGAAAGCUCUGAUUGGGGCUC | |
| | AGCUCGGACUGCUGACUGACGCCAAGAUCCAGAGGGCCGCAGCUGCCGUG | |
| | GAUCUGGGGAUCAAGGCCACUCUGGCCGCUACUAUUAUUCCUAACGCUCU | |
| | GGGGUCUGCCGCCUUCAAGAAUGCUGUCAUUGCCAACCUGGUGGCUGCUG | |
| | GCAUCGACAAACACCUCGCCAGGGCUACCGCUGUGGCCAUCGUUGCCACAG | |
| | CCCUGAAUCCUGCCCUGGGCCCAAUCGCUAAAUUUGAGCUGAUUAAGGCU | |
| | GAGAUCGCCGCCCAGGCCGCACUGCUGAUCAGAAGGGGGGUCCAUCUGCA | |
| | GAAGGCCGCUAUUGAACAUGUGAUCGGCCGCGCAUUCGACGCUGCCGUGG | |
| | CCACCGCUAUCAUUUCCUCUCCAAUCCUGAGCGCACGGAUUGUGACCCACC | |
| | UGGUGAGGGCUGGAAUUGAUAAAAGCAUCGCUAUCUCCCUGGCUCCUCAU | |
| | AUCGUGAAGCGCCUGGCCAAGGAGCCCCUGCUGGCUUUCAACACCGCCAA | |
| | GCUGAUGAAGAAUAUUACAAGACAGAUCGUUGACGUGAUCACAGCCGAUA | |
| | AAGCCAUCAAAACAGCUGAGCAGCUGGAAAAGGAGCUCCCCGCCCUGGAC | |
| | GACCUGGUCAAAAAAGCUAGCUCCCCUCCUAAGCCUACCCCCACACCUACU | |
| | CCAACCCCCACCGCCGGGCCCACCGUGACAGUGAUCCCCGUGGGAAGGGAG | |
| | GGGGGCGACAUUACCAUUAGCGGAAAAGGGUUCUCCACAACCGGGUUCGG | |
| | CGUCUACGUGGCCGUGGCUCCUGCCUCUGUGCCAGAGUUCUAUGGCAAUA | |
| | GCGACAAGUUCUAUGGGUACGAUCCAUCUAAGGACACCACCGAAAGUCCC | |
| | AGCACCAUCUGGGUGUAUACACCAUCCCAGAAAGCAAUCGGCAGCAGAUU | |
| | CGCCCAAGGCAGGCCCAUGAAUAAUGAUGGCUCUUUCACCAUUACAAUGA | |
| | AGGCCCCUCCUUUCGAGCAGGGAAAGGAUUUCGUGGUGCUGACCACCAAA | |
| | GCACAUGGCGUUGGAAAGACCGACCACAGCGACGAUACUCGGACCCCUGU | |
| | GACUUACAGAGAGGCCACCCCUGCUCCAACCGGCCCUAAAACUCCCAUCUG | |
| | AUAA | |
| 191 | UCCAGCAACCGGCCUAGAAGCGUGGCCCAGGCCGCCAUCGCCACCGACGGC | 82 |
| | AAGGGCAUCAUCGACAAGGAUUCCCGGGACGCCGUGAUCAAUGACGCCAA | |
| | GCUGAGAGCCGCCAUCGCCGGCGCCCUGGUGAAGGCCGGCUUCAGCAGCGC | |
| | CGACGCCGUGGCCCUGGCCCCCAGAAUCGCCAAGGAGAUGGCCAAGGAAG | |
| | GCGUGCUGCUGAUCAAUCACCACAAGCUGAAGGCCCUGAUCGGCGCCCAG | |
| | CUGGGCCUGCUGACAGAUGCCAAGAUCCAGAGAGCCGCUGCCGCCGUGGA | |
| | CCUGGGCAUCAAGGCCACCCUGGCCGCCACCAUCAUCCCUAACGCCCUGGG | |
| | CAGCGCUGCCUUCAAGAAUGCCGUGAUCGCCAACCUGGUGGCCGCCGGCA | |
| | UCGACAAGCACCUGGCCAGAGCCACAGCCGUGGCCAUCGUGGCCACCGCCC | |
| | UGAAUCCUGCCCUGGGCCCCAUCGCCAAGUUCGAGCUGAUCAAGGCCGAG | |
| | AUCGCCGCCCAGGCCGCCCUGCUGAUCCGGGGGGCGUGCACCUGCAGAAG | |
| | GCCGCCAUCGAACACGUGAUCGGCAGAGCCUUCGAUGCCGCCGUGGCCACC | |
| | GCCAUCAUCAGCUCCCCCAUCCUGAGCGCCAGAAUCGUGACCCACCUGGUG | |
| | CGGGCCGGCAUCGACAAGUCCAUCGCCAUUAGCCUGGCCCCUCACAUCGUG | |
| | AAGCGCCUGGCCAAGGAGCCCCUGCUGGCCUUCAAUACCGCCAAGCUGAU | |
| | GAAGAAUAUCACCAGACAGAUCGUGGAUGUGAUCACCGCCGAUAAGGCCA | |
| | UCAAGACCGCCGAGCAGCUGGAGAAAGAGCUGCCCGCCCUGGACGACCUG | |
| | GUGAAAAAGGCCAUGGGCCCCCCUAAGCCUACAGGCGGCGGCGGAGGCGC | |
| | UGGACCUACCGUGACCGUGAUCCCCGUGGGCAGAGAGGGCGGCGAUAUCA | |
| | CCAUCAGCGGCAAGGGCUUCAGCACCACCGGCUUCGGCGUGUACGUGGCC | |
| | GUGGCCCCUGCCAGCGUGCCCGAGUUCUACGGCAAUAGCGACAAGUUCUA | |
| | CGGCUACGACCCCAGCAAGGACACCACAGAAUCUCCCAGCACCAUCUGGGU | |
| | GUACACCCCCAGCCAGAAGGCCAUUGGCAGCCGGUUCGCCCAGGGCCGGCC | |
| | UAUGAACAAUGAUGGCAGCUUCACAAUCACCAUGAAGGCCCCACCCUUCG | |
| | AGCAGGGCAAGGACUUCGUGGUGCUGACCACCAAGGCCCACGGAGUGGGC | |
| | AAGGGCGACCACGGCGACGACACACGGACCCCCGUGGGCUACAGAUGA | |
| 192 | AGCAGCAACCGCCCAAGGUCUGUCGCACAGGCUGCUAUCGCCACAGAUGG | 83 |
| | CAAGGGAAUCAUUGAUAAGGACAGCAGGGAUGCUGUGAUCAACGACGCAA | |
| | AACUGAGAGCCGCCAUCGCCGGCGCACUGGUGAAAGCCGGCUUUUCUUCC | |
| | GCCGACGCAGUGGCUCUGGCUCCCAGAAUCGCUAAGGAAAUGGCCAAGGA | |
| | GGGAGUGCUGCUGAUCAAUCACCACAAACUGAAGGCUCUGAUCGGGGCUC | |
| | AGCUGGGCCUGCUGACUGACGCUAAGAUUCAGAGAGCAGCAGCAGCUGUC | |
| | GACCUGGGAAUCAAGGCAACCCUGGCAGCCACUAUCAUUCCAAAUGCUCU | |
| | GGGCUCUGCCGCCUUCAAAAACGCCGUGAUUGCCAAUCUCGUGGCAGCUG | |
| | GAAUCGAUAAGCACCUGGCACGGGCCACCGCCGUGGCUAUCGUGGCCACC | |
| | GCUCUGAAUCCCGCCCUGGGCCCUAUUGCCAAGUUUGAACUGAUCAAAGC | |
| | CGAGAUCGCUGCACAGGCCGCCCUGCUCAUCCGCAGGGGGGUCCACCUGCA | |
| | GAAAGCAGCCAUCGAGCACGUGAUCGGCAGGGCCUUUGAUGCAGCCGUCG | |
| | CCACUGCCAUCAUCAGCUCUCCAAUCCUGUCUGCCCGGAUUGUGACUCACC | |
| | UGGUGCGCGCCGGCAUCGAUAAGUCCAUCGCAAUCUCUCUGGCCCCACACA | |
| | UCGUGAAGAGACUGGCCAAAGAACCACUCCUGGCUUUCAAUACCGCCAAA | |
| | CUCAUGAAAAACAUCACCCGGCAGAUCGUCGAUGUGAUUACCGCCGACAA | |
| | GGCUAUCAAGACCGCCGAGCAGCUCGAAAAGGAGCUGCCUGCCCUGGACG | |
| | ACCUGGUGAAGAAAGCCUCCUCUCCACCCAAACCCACCCCAACUCCAACUC | |
| | CAACCCCUACAGCUGGCCCCACCGUGACCGUCAUCCCAGUGGGAAGGGAAG | |
| | GAGGAGACAUCACAAUCUCUGGAAAGGGGUUUAGCACAACCGGAUUUGGA | |
| | GUGUACGUCGCCGUGGCCCCCGCAUCCGUGCCAGAAUUCUACGGCAAUUCC | |
| | GAUAAAUUCUACGGGUACGACCCCUCCAAGGACACUACAGAGAGCCCCUC | |
| | UACAAUUUGGGUGUACACCCCAAGCCAGAAGGCCAUCGGAAGCAGAUUUG | |
| | CUCAGGGCCGGCCAAUGAACAACGACGGAUCCUUCACAAUCACAAUGAAA | |
| | GCACCUCCCUUUGAGCAGGGCAAGGAUUUUGUGGUGCUGACCACCAAGGC | |
| | UCACGGGGUCGGAAAGACCGAUCACAGCGACGAUACAAGAACCCCCGUGA | |
| | CAUACCGGUGAUAA | |
| 311 | | 42 |
| 312 | | 86 |
| 384 | GUGGAGCCCACAACCACAAUCUCUGCCACAAGCACCCACGAGCUGUCUGCC | 373 |
| | AGCGACGCCAGAAACUCCAUCCAGCUGCUGAAUGCCCACAUCGCCACACUG | |
| | CAGAGCGUGCAGAAAUCCGUGCCUGGCAGCGAUUACAGCGACCAGAUCCG | |
| | GGACCUGCUGAAGGCCGCCUUUGAUCUGAGAGGCCUGAUCGAAACCCUGG | |
| | CCCACGGCGGCAUUCCCUUCUAUGACCCCUCCACCAUCAUGCCCAGAAUCA | |
| | AGCUGGUGGCCACCACCAUCGACACCAUCCACACAGCUACAACCACCCUGC | |
| | AGAACAAGGUGCGGCCAGCCCAUGUGGAACUGGGCCUGGAGGUGACAAAG | |
| | GCCGUGCUGCUGACCGCCAAUCCUGCCAGCACAGCAAAAGAACUGGACGCC | |
| | GAGGGAGCUGCUCUGAAGGCCAGACUGGAAAAGGUGAGCCAGUACCCCGA | |
| | CCUGACACCUAAUGAUGUGGCCACAGUGUACGUGAGAACCAAUUUCAGCA | |
| | AGACCAUCUGGCAGGUGAGAGCCAACAGAGAUCGGUACAUCCUGGGCCAC | |
| | AAGUCCGCCGCCGUGUACAAGACCCUGAAUCACGCCAUCACCAAGGCCGUG | |
| | GGCGUGCGGCUGAAUCCUAAGACCACCGUGGGCAACAUCCAGGCCGCCAG | |
| | AACCGAACUGCUGGCCGCCUACCAGACCGCCUUCAACAGCCCCGAUGUGAA | |
| | GAAGGCCGCUAGCUCCAAUAGACCCAGAUCCGUGGCCCAGGCCGCCAUCGC | |
| | CACAGAUGGCAAGGGGAUCAUCGACAAGGAUUCCAGAGACGCUGUGAUCA | |
| | ACGACGCAAAGCUGAGAGCCGCCAUCGCCGGAGCUCUGGUGAAGGCCGGC | |
| | UUUAGCAGCGCCGACGCCGUGGCACUGGCCCCCAGAAUUGCAAAGGAGAU | |
| | GGCAAAGGAGGGCGUGCUGCUGAUCAAUCACCACAAGCUGAAGGCCCUGA | |
| | UCGGCGCACAGCUGGGCCUGCUGACCGACGCAAAGAUUCAGAGAGCCGCC | |
| | GCAGCCGUGGACCUGGGCAUCAAGGCCACCCUGGCUGCCACCAUCAUCCCU | |
| | AACGCCCUGGGCAGCGCCGCCUUCAAGAAUGCAGUGAUCGCCAACCUGGU | |
| | GGCCGCCGGAAUCGACAAGCACCUGGCCAGAGCCACAGCCGUCGCUAUCGU | |
| | GGCCACAGCCCUGAAUCCCGCCCUGGGCCCCAUCGCCAAAUUUGAACUGAU | |
| | CAAGGCAGAAAUCGCUGCCCAGGCCGCCCUGCUCAUCAGAAGAGGCGUCC | |
| | AUCUGCAGAAGGCCGCCAUCGAGCACGUGAUUGGCAGAGCCUUCGACGCC | |
| | GCCGUGGCCACAGCUAUCAUUUCUUCCCCUAUCCUGUCUGCCAGAAUCGU | |
| | GACACACCUGGUGAGAGCCGGAAUCGACAAGUCCAUCGCCAUCUCCCUGG | |
| | CCCCCCACAUCGUGAAGAGACUGGCUAAGGAGCCUCUGCUGGCCUUCAAU | |
| | ACAGCCAAGCUGAUGAAGAACAUCACAAGACAGAUCGUGGACGUGAUCAC | |
| | CGCCGACAAGGCCAUCAAGACAGCCGAGCAGCUGGAGAAGGAACUGCCAG | |
| | CCCUGGACGACCUGGUGAAGAAGGCCAGCAGCCCUCCCAAGCCUACCCCUA | |
| | CCCCAACCCCUACACCUACAGCCGGACCUACCGUGACCGUGAUCCCUGUGG | |
| | GCAGAGAGGGCGGCGACAUCACCAUCAGCGGCAAGGGCUUCAGCACAACA | |
| | GGCUUUGGCGUGUACGUGGCCGUGGCCCCAGCCAGCGUGCCUGAGUUUUA | |
| | CGGCAAUAGCGACAAGUUCUACGGCUACGACCCCUCCAAGGACACCACCGA | |
| | GAGCCCAAGCACCAUCUGGGUGUACACACCUAGCCAGAAGGCCAUCGGCA | |
| | GCAGAUUUGCCCAGGGCAGGCCUAUGAAUAAUGACGGCAGCUUCACAAUC | |
| | ACAAUGAAGGCCCCCCCCUUUGAGCAGGGCAAGGACUUCGUGGUGCUGAC | |
| | CACCAAGGCCCACGGCGUGGGCAAGACAGAUCACAGCGACGACACCCGGAC | |
| | CCCCGUGACCUACAGAGAAGCAACACCUGCCCCUACCGGGCCUAAGACACC | |
| | CAUC | |
| 385 | GUGGAGCCUACCACCACAAUCAGCGCCACCAGCACCCACGAGCUGAGCGCC | 373 |
| | AGCGAUGCCAGAAAUAGCAUCCAGCUGCUGAAUGCCCACAUCGCCACCCU | |
| | GCAGUCCGUGCAGAAAAGCGUGCCUGGAAGCGAUUACAGCGAUCAGAUCA | |
| | GGGACCUGCUGAAGGCCGCCUUCGAUCUGAGAGGCCUGAUCGAGACACUG | |
| | GCCCACGGCGGCAUCCCUUUCUACGACCCCAGCACCAUCAUGCCUAGAAUC | |
| | AAACUGGUGGCCACCACCAUCGACACCAUCCACACCGCAACAACAACCCUG | |
| | CAGAAUAAGGUGCGGCCUGCCCACGUGGAACUGGGACUGGAAGUGACAAA | |
| | GGCCGUGCUGCUGACCGCUAAUCCCGCCUCCACCGCCAAGGAGCUGGAUGC | |
| | CGAAGGAGCUGCCCUGAAAGCCAGACUGGAGAAGGUGAGCCAGUACCCUG | |
| | AUCUGACCCCUAACGACGUGGCCACCGUGUACGUGAGAACCAAUUUCAGC | |
| | AAGACCAUCUGGCAGGUGAGAGCCAAUCGGGACAGAUACAUCCUGGGCCA | |
| | CAAGUCUGCCGCCGUGUAUAAAACCCUGAAUCACGCCAUCACCAAGGCCG | |
| | UGGGCGUGAGACUGAACCCUAAGACAACCGUGGGCAACAUCCAGGCCGCC | |
| | AGAACAGAGCUGCUGGCCGCCUACCAGACAGCCUUCAAUAGCCCCGACGU | |
| | GAAGAAGGCCGCUUCCAGCAAUAGACCCCGGAGCGUGGCACAGGCUGCUA | |
| | UUGCCACCGACGGAAAGGGCAUCAUCGAUAAAGACAGCCGGGAUGCCGUG | |
| | AUCAAUGACGCCAAGCUGAGAGCAGCAAUCGCCGGCGCUCUGGUGAAGGC | |
| | AGGCUUCAGCAGCGCCGACGCCGUGGCUCUGGCCCCUAGAAUCGCUAAGG | |
| | AGAUGGCCAAGGAGGGCGUGCUGCUGAUCAAUCACCACAAGCUGAAGGCC | |
| | CUGAUCGGCGCCCAGCUGGGACUGCUGACAGAUGCCAAGAUCCAGAGAGC | |
| | CGCUGCAGCCGUGGAUCUGGGCAUCAAGGCCACACUGGCCGCCACCAUCAU | |
| | CCCAAACGCCCUGGGCAGCGCCGCCUUCAAAAAUGCCGUGAUUGCCAACCU | |
| | GGUGGCCGCCGGCAUCGAUAAGCACCUGGCCAGAGCCACAGCCGUGGCCA | |
| | UCGUGGCCACAGCCCUGAAUCCCGCACUGGGACCUAUUGCCAAGUUCGAA | |
| | CUCAUUAAGGCCGAAAUCGCAGCCCAAGCCGCCCUGCUGAUUCGGCGGGG | |
| | CGUGCACCUGCAGAAAGCCGCCAUUGAGCACGUGAUCGGCAGAGCCUUCG | |
| | AUGCCGCUGUGGCCACCGCCAUCAUCUCCUCUCCUAUCCUGUCUGCUAGAA | |
| | UCGUGACCCACCUGGUCAGAGCCGGAAUCGACAAGUCUAUCGCUAUCAGC | |
| | CUGGCCCCCCACAUCGUGAAGAGACUGGCCAAAGAGCCCCUGCUGGCCUUC | |
| | AACACAGCCAAGCUCAUGAAGAAUAUCACCAGACAGAUCGUGGACGUGAU | |
| | CACCGCCGACAAGGCCAUCAAAACCGCUGAGCAGCUGGAGAAGGAGCUGC | |
| | CCGCCCUGGAUGACCUGGUGAAGAAGGCCAGCAGCCCCCCCAAGCCUACAC | |
| | CUACACCUACCCCAACCCCUACAGCCGGCCCUACAGUGACCGUGAUUCCUG | |
| | UGGGCAGAGAAGGAGGCGACAUCACCAUCAGCGGAAAGGGCUUCUCUACC | |
| | ACAGGCUUCGGCGUGUACGUGGCCGUGGCCCCUGCCUCCGUGCCCGAAUUC | |
| | UACGGCAACAGCGACAAGUUCUACGGCUACGACCCCAGCAAAGACACCAC | |
| | AGAGAGCCCUUCCACCAUCUGGGUGUACACCCCUUCCCAGAAGGCCAUCGG | |
| | CAGCAGAUUCGCUCAGGGCAGACCUAUGAAUAACGACGGCUCUUUCACCA | |
| | UCACAAUGAAGGCCCCCCCCUUCGAGCAGGGCAAAGACUUCGUGGUGCUG | |
| | ACAACCAAGGCCCACGGCGUGGGCAAGACAGAUCACUCCGACGACACCCGG | |
| | ACCCCUGUGACCUACAGAGAGGCAACACCUGCUCCCACCGGGCCAAAGACC | |
| | CCUAUC | |
| 386 | AUGGUGGAGCCCACAACCACAAUCUCUGCCACAAGCACCCACGAGCUGUCU | 374 |
| | GCCAGCGACGCCAGAAACUCCAUCCAGCUGCUGAAUGCCCACAUCGCCACA | |
| | CUGCAGAGCGUGCAGAAAUCCGUGCCUGGCAGCGAUUACAGCGACCAGAU | |
| | CCGGGACCUGCUGAAGGCCGCCUUUGAUCUGAGAGGCCUGAUCGAAACCC | |
| | UGGCCCACGGCGGCAUUCCCUUCUAUGACCCCUCCACCAUCAUGCCCAGAA | |
| | UCAAGCUGGUGGCCACCACCAUCGACACCAUCCACACAGCUACAACCACCC | |
| | UGCAGAACAAGGUGCGGCCAGCCCAUGUGGAACUGGGCCUGGAGGUGACA | |
| | AAGGCCGUGCUGCUGACCGCCAAUCCUGCCAGCACAGCAAAAGAACUGGA | |
| | CGCCGAGGGAGCUGCUCUGAAGGCCAGACUGGAAAAGGUGAGCCAGUACC | |
| | CCGACCUGACACCUAAUGAUGUGGCCACAGUGUACGUGAGAACCAAUUUC | |
| | AGCAAGACCAUCUGGCAGGUGAGAGCCAACAGAGAUCGGUACAUCCUGGG | |
| | CCACAAGUCCGCCGCCGUGUACAAGACCCUGAAUCACGCCAUCACCAAGGC | |
| | CGUGGGCGUGCGGCUGAAUCCUAAGACCACCGUGGGCAACAUCCAGGCCG | |
| | CCAGAACCGAACUGCUGGCCGCCUACCAGACCGCCUUCAACAGCCCCGAUG | |
| | UGAAGAAGGCCGCUAGCUCCAAUAGACCCAGAUCCGUGGCCCAGGCCGCC | |
| | AUCGCCACAGAUGGCAAGGGGAUCAUCGACAAGGAUUCCAGAGACGCUGU | |
| | GAUCAACGACGCAAAGCUGAGAGCCGCCAUCGCCGGAGCUCUGGUGAAGG | |
| | CCGGCUUUAGCAGCGCCGACGCCGUGGCACUGGCCCCCAGAAUUGCAAAG | |
| | GAGAUGGCAAAGGAGGGCGUGCUGCUGAUCAAUCACCACAAGCUGAAGGC | |
| | CCUGAUCGGCGCACAGCUGGGCCUGCUGACCGACGCAAAGAUUCAGAGAG | |
| | CCGCCGCAGCCGUGGACCUGGGCAUCAAGGCCACCCUGGCUGCCACCAUCA | |
| | UCCCUAACGCCCUGGGCAGCGCCGCCUUCAAGAAUGCAGUGAUCGCCAACC | |
| | UGGUGGCCGCCGGAAUCGACAAGCACCUGGCCAGAGCCACAGCCGUCGCU | |
| | AUCGUGGCCACAGCCCUGAAUCCCGCCCUGGGCCCCAUCGCCAAAUUUGAA | |
| | CUGAUCAAGGCAGAAAUCGCUGCCCAGGCCGCCCUGCUCAUCAGAAGAGG | |
| | CGUCCAUCUGCAGAAGGCCGCCAUCGAGCACGUGAUUGGCAGAGCCUUCG | |
| | ACGCCGCCGUGGCCACAGCUAUCAUUUCUUCCCCUAUCCUGUCUGCCAGAA | |
| | UCGUGACACACCUGGUGAGAGCCGGAAUCGACAAGUCCAUCGCCAUCUCC | |
| | CUGGCCCCCCACAUCGUGAAGAGACUGGCUAAGGAGCCUCUGCUGGCCUU | |
| | CAAUACAGCCAAGCUGAUGAAGAACAUCACAAGACAGAUCGUGGACGUGA | |
| | UCACCGCCGACAAGGCCAUCAAGACAGCCGAGCAGCUGGAGAAGGAACUG | |
| | CCAGCCCUGGACGACCUGGUGAAGAAGGCCAGCAGCCCUCCCAAGCCUACC | |
| | CCUACCCCAACCCCUACACCUACAGCCGGACCUACCGUGACCGUGAUCCCU | |
| | GUGGGCAGAGAGGGCGGCGACAUCACCAUCAGCGGCAAGGGCUUCAGCAC | |
| | AACAGGCUUUGGCGUGUACGUGGCCGUGGCCCCAGCCAGCGUGCCUGAGU | |
| | UUUACGGCAAUAGCGACAAGUUCUACGGCUACGACCCCUCCAAGGACACC | |
| | ACCGAGAGCCCAAGCACCAUCUGGGUGUACACACCUAGCCAGAAGGCCAU | |
| | CGGCAGCAGAUUUGCCCAGGGCAGGCCUAUGAAUAAUGACGGCAGCUUCA | |
| | CAAUCACAAUGAAGGCCCCCCCCUUUGAGCAGGGCAAGGACUUCGUGGUG | |
| | CUGACCACCAAGGCCCACGGCGUGGGCAAGACAGAUCACAGCGACGACACC | |
| | CGGACCCCCGUGACCUACAGAGAAGCAACACCUGCCCCUACCGGGCCUAAG | |
| | ACACCCAUC | |
| 387 | GUGGAGCCUACCACCACAAUCAGCGCCACCAGCACCCACGAGCUGAGCGCC | 374 |
| | AGCGAUGCCAGAAAUAGCAUCCAGCUGCUGAAUGCCCACAUCGCCACCCU | |
| | GCAGUCCGUGCAGAAAAGCGUGCCUGGAAGCGAUUACAGCGAUCAGAUCA | |
| | GGGACCUGCUGAAGGCCGCCUUCGAUCUGAGAGGCCUGAUCGAGACACUG | |
| | GCCCACGGCGGCAUCCCUUUCUACGACCCCAGCACCAUCAUGCCUAGAAUC | |
| | AAACUGGUGGCCACCACCAUCGACACCAUCCACACCGCAACAACAACCCUG | |
| | CAGAAUAAGGUGCGGCCUGCCCACGUGGAACUGGGACUGGAAGUGACAAA | |
| | GGCCGUGCUGCUGACCGCUAAUCCCGCCUCCACCGCCAAGGAGCUGGAUGC | |
| | CGAAGGAGCUGCCCUGAAAGCCAGACUGGAGAAGGUGAGCCAGUACCCUG | |
| | AUCUGACCCCUAACGACGUGGCCACCGUGUACGUGAGAACCAAUUUCAGC | |
| | AAGACCAUCUGGCAGGUGAGAGCCAAUCGGGACAGAUACAUCCUGGGCCA | |
| | CAAGUCUGCCGCCGUGUAUAAAACCCUGAAUCACGCCAUCACCAAGGCCG | |
| | UGGGCGUGAGACUGAACCCUAAGACAACCGUGGGCAACAUCCAGGCCGCC | |
| | AGAACAGAGCUGCUGGCCGCCUACCAGACAGCCUUCAAUAGCCCCGACGU | |
| | GAAGAAGGCCGCUUCCAGCAAUAGACCCCGGAGCGUGGCACAGGCUGCUA | |
| | UUGCCACCGACGGAAAGGGCAUCAUCGAUAAAGACAGCCGGGAUGCCGUG | |
| | AUCAAUGACGCCAAGCUGAGAGCAGCAAUCGCCGGCGCUCUGGUGAAGGC | |
| | AGGCUUCAGCAGCGCCGACGCCGUGGCUCUGGCCCCUAGAAUCGCUAAGG | |
| | AGAUGGCCAAGGAGGGCGUGCUGCUGAUCAAUCACCACAAGCUGAAGGCC | |
| | CUGAUCGGCGCCCAGCUGGGACUGCUGACAGAUGCCAAGAUCCAGAGAGC | |
| | CGCUGCAGCCGUGGAUCUGGGCAUCAAGGCCACACUGGCCGCCACCAUCAU | |
| | CCCAAACGCCCUGGGCAGCGCCGCCUUCAAAAAUGCCGUGAUUGCCAACCU | |
| | GGUGGCCGCCGGCAUCGAUAAGCACCUGGCCAGAGCCACAGCCGUGGCCA | |
| | UCGUGGCCACAGCCCUGAAUCCCGCACUGGGACCUAUUGCCAAGUUCGAA | |
| | CUCAUUAAGGCCGAAAUCGCAGCCCAAGCCGCCCUGCUGAUUCGGCGGGG | |
| | CGUGCACCUGCAGAAAGCCGCCAUUGAGCACGUGAUCGGCAGAGCCUUCG | |
| | AUGCCGCUGUGGCCACCGCCAUCAUCUCCUCUCCUAUCCUGUCUGCUAGAA | |
| | UCGUGACCCACCUGGUCAGAGCCGGAAUCGACAAGUCUAUCGCUAUCAGC | |
| | CUGGCCCCCCACAUCGUGAAGAGACUGGCCAAAGAGCCCCUGCUGGCCUUC | |
| | AACACAGCCAAGCUCAUGAAGAAUAUCACCAGACAGAUCGUGGACGUGAU | |
| | CACCGCCGACAAGGCCAUCAAAACCGCUGAGCAGCUGGAGAAGGAGCUGC | |
| | CCGCCCUGGAUGACCUGGUGAAGAAGGCCAGCAGCCCCCCCAAGCCUACAC | |
| | CUACACCUACCCCAACCCCUACAGCCGGCCCUACAGUGACCGUGAUUCCUG | |
| | UGGGCAGAGAAGGAGGCGACAUCACCAUCAGCGGAAAGGGCUUCUCUACC | |
| | ACAGGCUUCGGCGUGUACGUGGCCGUGGCCCCUGCCUCCGUGCCCGAAUUC | |
| | UACGGCAACAGCGACAAGUUCUACGGCUACGACCCCAGCAAAGACACCAC | |
| | AGAGAGCCCUUCCACCAUCUGGGUGUACACCCCUUCCCAGAAGGCCAUCGG | |
| | CAGCAGAUUCGCUCAGGGCAGACCUAUGAAUAACGACGGCUCUUUCACCA | |
| | UCACAAUGAAGGCCCCCCCCUUCGAGCAGGGCAAAGACUUCGUGGUGCUG | |
| | ACAACCAAGGCCCACGGCGUGGGCAAGACAGAUCACUCCGACGACACCCGG | |
| | ACCCCUGUGACCUACAGAGAGGCAACACCUGCUCCCACCGGGCCAAAGACC | |
| | CCUAUC | |
| 388 | GUGGAGCCCACAACCACAAUCUCUGCCACAAGCACCCACGAGCUGUCUGCC | 375 |
| | AGCGACGCCAGAAACUCCAUCCAGCUGCUGAAUGCCCACAUCGCCACACUG | |
| | CAGAGCGUGCAGAAAUCCGUGCCUGGCAGCGAUUACAGCGACCAGAUCCG | |
| | GGACCUGCUGAAGGCCGCCUUUGAUCUGAGAGGCCUGAUCGAAACCCUGG | |
| | CCCACGGCGGCAUUCCCUUCUAUGACCCCUCCACCAUCAUGCCCAGAAUCA | |
| | AGCUGGUGGCCACCACCAUCGACACCAUCCACACAGCUACAACCACCCUGC | |
| | AGAACAAGGUGCGGCCAGCCCAUGUGGAACUGGGCCUGGAGGUGACAAAG | |
| | GCCGUGCUGCUGACCGCCAAUCCUGCCAGCACAGCAAAAGAACUGGACGCC | |
| | GAGGGAGCUGCUCUGAAGGCCAGACUGGAAAAGGUGAGCCAGUACCCCGA | |
| | CCUGACACCUAAUGAUGUGGCCACAAGCUCCAAUAGACCCAGAUCCGUGG | |
| | CCCAGGCCGCCAUCGCCACAGAUGGCAAGGGGAUCAUCGACAAGGAUUCC | |
| | AGAGACGCUGUGAUCAACGACGCAAAGCUGAGAGCCGCCAUCGCCGGAGC | |
| | UCUGGUGAAGGCCGGCUUUAGCAGCGCCGACGCCGUGGCACUGGCCCCCA | |
| | GAAUUGCAAAGGAGAUGGCAAAGGAGGGCGUGCUGCUGAUCAAUCACCAC | |
| | AAGCUGAAGGCCCUGAUCGGCGCACAGCUGGGCCUGCUGACCGACGCAAA | |
| | GAUUCAGAGAGCCGCCGCAGCCGUGGACCUGGGCAUCAAGGCCACCCUGG | |
| | CUGCCACCAUCAUCCCUAACGCCCUGGGCAGCGCCGCCUUCAAGAAUGCAG | |
| | UGAUCGCCAACCUGGUGGCCGCCGGAAUCGACAAGCACCUGGCCAGAGCC | |
| | ACAGCCGUCGCUAUCGUGGCCACAGCCCUGAAUCCCGCCCUGGGCCCCAUC | |
| | GCCAAAUUUGAACUGAUCAAGGCAGAAAUCGCUGCCCAGGCCGCCCUGCU | |
| | CAUCAGAAGAGGCGUCCAUCUGCAGAAGGCCGCCAUCGAGCACGUGAUUG | |
| | GCAGAGCCUUCGACGCCGCCGUGGCCACAGCUAUCAUUUCUUCCCCUAUCC | |
| | UGUCUGCCAGAAUCGUGACACACCUGGUGAGAGCCGGAAUCGACAAGUCC | |
| | AUCGCCAUCUCCCUGGCCCCCCACAUCGUGAAGAGACUGGCUAAGGAGCCU | |
| | CUGCUGGCCUUCAAUACAGCCAAGCUGAUGAAGAACAUCACAAGACAGAU | |
| | CGUGGACGUGAUCACCGCCGACAAGGCCAUCAAGACAGCCGAGCAGCUGG | |
| | AGAAGGAACUGCCAGCCCUGGACGACCUGGUGAAGAAGGCCAGCAGCCCU | |
| | CCCAAGCCUACCCCUACCCCAACCCCUACACCUACAGCCGGACCUACCGUG | |
| | ACCGUGAUCCCUGUGGGCAGAGAGGGCGGCGACAUCACCAUCAGCGGCAA | |
| | GGGCUUCAGCACAACAGGCUUUGGCGUGUACGUGGCCGUGGCCCCAGCCA | |
| | GCGUGCCUGAGUUUUACGGCAAUAGCGACAAGUUCUACGGCUACGACCCC | |
| | UCCAAGGACACCACCGAGAGCCCAAGCACCAUCUGGGUGUACACACCUAGC | |
| | CAGAAGGCCAUCGGCAGCAGAUUUGCCCAGGGCAGGCCUAUGAAUAAUGA | |
| | CGGCAGCUUCACAAUCACAAUGAAGGCCCCCCCCUUUGAGCAGGGCAAGG | |
| | ACUUCGUGGUGCUGACCACCAAGGCCCACGGCGUGGGCAAGACAGAUCAC | |
| | AGCGACGACACCCGGACCCCCGUGACCUACAGAGAAGCAACACCUGCCCCU | |
| | ACCGGGCCUAAGACACCCAUC | |
| 389 | GUGGAGCCUACCACCACAAUCAGCGCCACCAGCACCCACGAGCUGAGCGCC | 375 |
| | AGCGAUGCCAGAAAUAGCAUCCAGCUGCUGAAUGCCCACAUCGCCACCCU | |
| | GCAGUCCGUGCAGAAAAGCGUGCCUGGAAGCGAUUACAGCGAUCAGAUCA | |
| | GGGACCUGCUGAAGGCCGCCUUCGAUCUGAGAGGCCUGAUCGAGACACUG | |
| | GCCCACGGCGGCAUCCCUUUCUACGACCCCAGCACCAUCAUGCCUAGAAUC | |
| | AAACUGGUGGCCACCACCAUCGACACCAUCCACACCGCAACAACAACCCUG | |
| | CAGAAUAAGGUGCGGCCUGCCCACGUGGAACUGGGACUGGAAGUGACAAA | |
| | GGCCGUGCUGCUGACCGCUAAUCCCGCCUCCACCGCCAAGGAGCUGGAUGC | |
| | CGAAGGAGCUGCCCUGAAAGCCAGACUGGAGAAGGUGAGCCAGUACCCUG | |
| | AUCUGACCCCUAACGACGUGGCCACCUCCAGCAAUAGACCCCGGAGCGUGG | |
| | CACAGGCUGCUAUUGCCACCGACGGAAAGGGCAUCAUCGAUAAAGACAGC | |
| | CGGGAUGCCGUGAUCAAUGACGCCAAGCUGAGAGCAGCAAUCGCCGGCGC | |
| | UCUGGUGAAGGCAGGCUUCAGCAGCGCCGACGCCGUGGCUCUGGCCCCUA | |
| | GAAUCGCUAAGGAGAUGGCCAAGGAGGGCGUGCUGCUGAUCAAUCACCAC | |
| | AAGCUGAAGGCCCUGAUCGGCGCCCAGCUGGGACUGCUGACAGAUGCCAA | |
| | GAUCCAGAGAGCCGCUGCAGCCGUGGAUCUGGGCAUCAAGGCCACACUGG | |
| | CCGCCACCAUCAUCCCAAACGCCCUGGGCAGCGCCGCCUUCAAAAAUGCCG | |
| | UGAUUGCCAACCUGGUGGCCGCCGGCAUCGAUAAGCACCUGGCCAGAGCC | |
| | ACAGCCGUGGCCAUCGUGGCCACAGCCCUGAAUCCCGCACUGGGACCUAUU | |
| | GCCAAGUUCGAACUCAUUAAGGCCGAAAUCGCAGCCCAAGCCGCCCUGCU | |
| | GAUUCGGCGGGGCGUGCACCUGCAGAAAGCCGCCAUUGAGCACGUGAUCG | |
| | GCAGAGCCUUCGAUGCCGCUGUGGCCACCGCCAUCAUCUCCUCUCCUAUCC | |
| | UGUCUGCUAGAAUCGUGACCCACCUGGUCAGAGCCGGAAUCGACAAGUCU | |
| | AUCGCUAUCAGCCUGGCCCCCCACAUCGUGAAGAGACUGGCCAAAGAGCCC | |
| | CUGCUGGCCUUCAACACAGCCAAGCUCAUGAAGAAUAUCACCAGACAGAU | |
| | CGUGGACGUGAUCACCGCCGACAAGGCCAUCAAAACCGCUGAGCAGCUGG | |
| | AGAAGGAGCUGCCCGCCCUGGAUGACCUGGUGAAGAAGGCCAGCAGCCCC | |
| | CCCAAGCCUACACCUACACCUACCCCAACCCCUACAGCCGGCCCUACAGUG | |
| | ACCGUGAUUCCUGUGGGCAGAGAAGGAGGCGACAUCACCAUCAGCGGAAA | |
| | GGGCUUCUCUACCACAGGCUUCGGCGUGUACGUGGCCGUGGCCCCUGCCUC | |
| | CGUGCCCGAAUUCUACGGCAACAGCGACAAGUUCUACGGCUACGACCCCA | |
| | GCAAAGACACCACAGAGAGCCCUUCCACCAUCUGGGUGUACACCCCUUCCC | |
| | AGAAGGCCAUCGGCAGCAGAUUCGCUCAGGGCAGACCUAUGAAUAACGAC | |
| | GGCUCUUUCACCAUCACAAUGAAGGCCCCCCCCUUCGAGCAGGGCAAAGAC | |
| | UUCGUGGUGCUGACAACCAAGGCCCACGGCGUGGGCAAGACAGAUCACUC | |
| | CGACGACACCCGGACCCCUGUGACCUACAGAGAGGCAACACCUGCUCCCAC | |
| | CGGGCCAAAGACCCCUAUC | |
| 390 | UCCAGCAAUAGACCCCGGAGCGUGGCACAGGCUGCUAUUGCCACCGACGG | 376 |
| | AAAGGGCAUCAUCGAUAAAGACAGCCGGGAUGCCGUGAUCAAUGACGCCA | |
| | AGCUGAGAGCAGCAAUCGCCGGCGCUCUGGUGAAGGCAGGCUUCAGCAGC | |
| | GCCGACGCCGUGGCUCUGGCCCCUAGAAUCGCUAAGGAGAUGGCCAAGGA | |
| | GGGCGUGCUGCUGAUCAAUCACCACAAGCUGAAGGCCCUGAUCGGCGCCC | |
| | AGCUGGGACUGCUGACAGAUGCCAAGAUCCAGAGAGCCGCUGCAGCCGUG | |
| | GAUCUGGGCAUCAAGGCCACACUGGCCGCCACCAUCAUCCCAAACGCCCUG | |
| | GGCAGCGCCGCCUUCAAAAAUGCCGUGAUUGCCAACCUGGUGGCCGCCGG | |
| | CAUCGAUAAGCACCUGGCCAGAGCCACAGCCGUGGCCAUCGUGGCCACAGC | |
| | CCUGAAUCCCGCACUGGGACCUAUUGCCAAGUUCGAACUCAUUAAGGCCG | |
| | AAAUCGCAGCCCAAGCCGCCCUGCUGAUUCGGCGGGGCGUGCACCUGCAG | |
| | AAAGCCGCCAUUGAGCACGUGAUCGGCAGAGCCUUCGAUGCCGCUGUGGC | |
| | CACCGCCAUCAUCUCCUCUCCUAUCCUGUCUGCUAGAAUCGUGACCCACCU | |
| | GGUCAGAGCCGGAAUCGACAAGUCUAUCGCUAUCAGCCUGGCCCCCCACA | |
| | UCGUGAAGAGACUGGCCAAAGAGCCCCUGCUGGCCUUCAACACAGCCAAG | |
| | CUCAUGAAGAAUAUCACCAGACAGAUCGUGGACGUGAUCACCGCCGACAA | |
| | GGCCAUCAAAACCGCUGAGCAGCUGGAGAAGGAGCUGCCCGCCCUGGAUG | |
| | ACCUGGUGAAGAAGGCCAGCAGCCCCCCCAAGCCUACACCUACACCUACCC | |
| | CAACCCCUACAGCCGGCCCUACAGUGACCGUGAUUCCUGUGGGCAGAGAA | |
| | GGAGGCGACAUCACCAUCAGCGGAAAGGGCUUCUCUACCACAGGCUUCGG | |
| | CGUGUACGUGGCCGUGGCCCCUGCCUCCGUGCCCGAAUUCUACGGCAACAG | |
| | CGACAAGUUCUACGGCUACGACCCCAGCAAAGACACCACAGAGAGCCCUUC | |
| | CACCAUCUGGGUGUACACCCCUUCCCAGAAGGCCAUCGGCAGCAGAUUCGC | |
| | UCAGGGCAGACCUAUGAAUAACGACGGCUCUUUCACCAUCACAAUGAAGG | |
| | CCCCCCCCUUCGAGCAGGGCAAAGACUUCGUGGUGCUGACAACCAAGGCCC | |
| | ACGGCGUGGGCAAGACAGAUCACUCCGACGACACCCGGACCCCUGUGACCU | |
| | ACAGAGAGGCAACACCUGCUCCCACCGGGCCAAAGACCCCUAUCGUGUACG | |
| | UGAGAACCAAUUUCAGCAAGACCAUCUGGCAGGUGAGAGCCAAUCGGGAC | |
| | AGAUACAUCCUGGGCCACAAGUCUGCCGCCGUGUAUAAAACCCUGAAUCA | |
| | CGCCAUCACCAAGGCCGUGGGCGUGAGACUGAACCCUAAGACAACCGUGG | |
| | GCAACAUCCAGGCCGCCAGAACAGAGCUGCUGGCCGCCUACCAGACAGCCU | |
| | UCAAUAGCCCCGACGUGAAGAAGGCCGCU | |
| 393 | AGCUCCAAUAGACCCAGAUCCGUGGCCCAGGCCGCCAUCGCCACAGAUGGC | 376 |
| | AAGGGGAUCAUCGACAAGGAUUCCAGAGACGCUGUGAUCAACGACGCAAA | |
| | GCUGAGAGCCGCCAUCGCCGGAGCUCUGGUGAAGGCCGGCUUUAGCAGCG | |
| | CCGACGCCGUGGCACUGGCCCCCAGAAUUGCAAAGGAGAUGGCAAAGGAG | |
| | GGCGUGCUGCUGAUCAAUCACCACAAGCUGAAGGCCCUGAUCGGCGCACA | |
| | GCUGGGCCUGCUGACCGACGCAAAGAUUCAGAGAGCCGCCGCAGCCGUGG | |
| | ACCUGGGCAUCAAGGCCACCCUGGCUGCCACCAUCAUCCCUAACGCCCUGG | |
| | GCAGCGCCGCCUUCAAGAAUGCAGUGAUCGCCAACCUGGUGGCCGCCGGA | |
| | AUCGACAAGCACCUGGCCAGAGCCACAGCCGUCGCUAUCGUGGCCACAGCC | |
| | CUGAAUCCCGCCCUGGGCCCCAUCGCCAAAUUUGAACUGAUCAAGGCAGA | |
| | AAUCGCUGCCCAGGCCGCCCUGCUCAUCAGAAGAGGCGUCCAUCUGCAGA | |
| | AGGCCGCCAUCGAGCACGUGAUUGGCAGAGCCUUCGACGCCGCCGUGGCC | |
| | ACAGCUAUCAUUUCUUCCCCUAUCCUGUCUGCCAGAAUCGUGACACACCU | |
| | GGUGAGAGCCGGAAUCGACAAGUCCAUCGCCAUCUCCCUGGCCCCCCACAU | |
| | CGUGAAGAGACUGGCUAAGGAGCCUCUGCUGGCCUUCAAUACAGCCAAGC | |
| | UGAUGAAGAACAUCACAAGACAGAUCGUGGACGUGAUCACCGCCGACAAG | |
| | GCCAUCAAGACAGCCGAGCAGCUGGAGAAGGAACUGCCAGCCCUGGACGA | |
| | CCUGGUGAAGAAGGCCAGCAGCCCUCCCAAGCCUACCCCUACCCCAACCCC | |
| | UACACCUACAGCCGGACCUACCGUGACCGUGAUCCCUGUGGGCAGAGAGG | |
| | GCGGCGACAUCACCAUCAGCGGCAAGGGCUUCAGCACAACAGGCUUUGGC | |
| | GUGUACGUGGCCGUGGCCCCAGCCAGCGUGCCUGAGUUUUACGGCAAUAG | |
| | CGACAAGUUCUACGGCUACGACCCCUCCAAGGACACCACCGAGAGCCCAAG | |
| | CACCAUCUGGGUGUACACACCUAGCCAGAAGGCCAUCGGCAGCAGAUUUG | |
| | CCCAGGGCAGGCCUAUGAAUAAUGACGGCAGCUUCACAAUCACAAUGAAG | |
| | GCCCCCCCCUUUGAGCAGGGCAAGGACUUCGUGGUGCUGACCACCAAGGCC | |
| | CACGGCGUGGGCAAGACAGAUCACAGCGACGACACCCGGACCCCCGUGACC | |
| | UACAGAGAAGCAACACCUGCCCCUACCGGGCCUAAGACACCCAUCGUGUAC | |
| | GUGAGAACCAAUUUCAGCAAGACCAUCUGGCAGGUGAGAGCCAACAGAGA | |
| | UCGGUACAUCCUGGGCCACAAGUCCGCCGCCGUGUACAAGACCCUGAAUC | |
| | ACGCCAUCACCAAGGCCGUGGGCGUGCGGCUGAAUCCUAAGACCACCGUG | |
| | GGCAACAUCCAGGCCGCCAGAACCGAACUGCUGGCCGCCUACCAGACCGCC | |
| | UUCAACAGCCCCGAUGUGAAGAAGGCCGCU | |
| 395 | AGCGGCAGCAUCCUGGCUAUCUACAGCACAGUGGCUUCUAGCCUGGUGCU | 84 |
| | GCUGGUGUCUCUGGGCGCCAUCAGCUUUGGCAGCGGC | |
| 396 | AGCGGAAGCAUCCUGGCCAUCUAUAGCACCGUGGCUAGCUCUCUGGUGCU | 84 |
| | GCUGGUGAGCCUGGGAGCUAUCAGCUUUGGGAGCGGC | |

1. A nucleic acid comprising a nucleotide sequence encoding a modified Cutibacterium acnes (C. acnes) Christie-Atkins-Munch-Petersen factor 2 (CAMP2) polypeptide, wherein the modified C. acnes CAMP2 polypeptide comprises an amino acid sequence comprising a C. acnes CAMP2 polypeptide sequence and a transmembrane domain sequence.

2. The nucleic acid of claim 1, wherein:
(A) (a) the transmembrane domain sequence is positioned at the C terminus of the modified C. acnes CAMP2 polypeptide; and/or
(b) the transmembrane domain sequence comprises a viral transmembrane domain sequence, which is optionally selected from the group consisting of: an influenza hemagglutinin (HA) transmembrane domain sequence, a SARS CoV-2 spike transmembrane domain sequence, a VZV gB transmembrane domain sequence, a VZV gE transmembrane domain sequence, a VZV gI transmembrane domain sequence, a VZV gK transmembrane domain sequence, a measles F-protein transmembrane domain sequence, a rubella E1 protein transmembrane domain sequence, a rubella E2 protein transmembrane domain sequence, a mumps F-protein transmembrane domain sequence, an Ebola GP protein transmembrane domain sequence and a rabies transmembrane domain sequence, optionally wherein the transmembrane domain comprises an amino acid sequence selected from the group consisting of sequences set forth by SEQ ID NO: 208-209 and 241-253; and/or
(B) the modified C. acnes CAMP2 polypeptide comprises a non-native secretion signal peptide sequence (e.g., a viral secretion signal peptide sequence), optionally wherein:
(a) the secretion signal peptide sequence is positioned at the N terminus of the modified C. acnes CAMP2 polypeptide; and/or
(b) the secretion signal peptide sequence is a viral secretion signal peptide sequence, which is optionally selected from the group consisting of: an influenza hemagglutinin (HA) secretion signal peptide sequence, a SARS CoV-2 spike secretion signal peptide sequence, a VZV gB secretion signal peptide sequence, a VZV gE secretion signal peptide sequence, a VZV gI secretion signal peptide sequence, a VZV gK secretion signal peptide sequence, a measles F-protein secretion signal peptide sequence, a rubella E1 protein secretion signal peptide sequence, a rubella E2 protein secretion signal peptide sequence, a mumps F-protein secretion signal peptide sequence, an Ebola GP protein secretion signal peptide sequence, a smallpox 6 kDa IC protein secretion signal peptide sequence and a rabies G protein secretion signal peptide sequence, e.g., wherein the secretion signal peptide sequence comprises a sequence selected from the group consisting of sequences set forth by SEQ ID NO: 208-209 and 241-253; and/or
wherein the nucleic acid is a messenger RNA (mRNA), optionally wherein
(a) the mRNA comprises a 5′ cap, at least one 5′ untranslated region (5′ UTR), at least one 3′ untranslated region (3′ UTR), and/or at least one polyadenylation (poly(A)) sequence;
(b) the mRNA is unmodified or comprises at least one chemical modification, optionally wherein the mRNA comprises at least one chemical modification, e.g., wherein the chemical modification comprises N1-methylpseudouridine; and/or
(c) the mRNA is a self-replicating mRNA or a non-replicating mRNA.

3. (canceled)

4. (canceled)

5. The nucleic acid of claim 1, wherein:
(a) the modified C. acnes CAMP2 polypeptide comprises a sequence according to any one of SEQ ID NO: 207 or SEQ ID NO: 5-9, or a sequence having at least 60% identity thereto; and/or
(b) the nucleic acid comprises a nucleotide sequence according to any one of SEQ ID NO: 90-94 or SEQ ID NO: 391-392, or a sequence having at least 50% identity thereto; and/or
(c) the nucleic acid is a mRNA comprising or consisting of the following structural elements:
(i) a 5′ cap with the following structure: 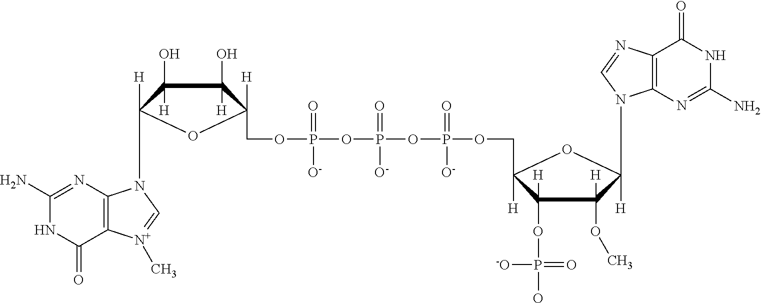
(ii) a 5′ untranslated region (5′ UTR) having the nucleic acid sequence according to SEQ ID NO: 265;
(iii) a protein coding region having the nucleic acid sequence according to any one of SEQ ID NO: 90-91 or SEQ ID NO: 391-392 (e.g., SEQ ID NO: 91);
(iv) a 3′ untranslated region (3′ UTR) having the nucleic acid sequence according to SEQ ID NO: 266; and
(v) a polyA tail, optionally wherein the poly A tail comprises at least 75 adenosine nucleotides or at least 100 adenosine nucleotides.

6. (canceled)

7. A modified C. acnes CAMP2 polypeptide having an amino acid sequence comprising a C. acnes CAMP2 polypeptide sequence and a transmembrane domain sequence.

8. The modified C. acnes CAMP2 polypeptide of claim 7, wherein:
(A) the transmembrane domain sequence is positioned at the C-terminus of the modified C. acnes CAMP2 polypeptide; and/or
(B) the transmembrane domain comprises a viral transmembrane domain sequence, which is optionally selected from the group consisting of: an influenza hemagglutinin (HA) transmembrane domain sequence, a SARS CoV-2 spike transmembrane domain sequence, a VZV gB transmembrane domain sequence, a VZV gE transmembrane domain sequence, a VZV gI transmembrane domain sequence, a VZV gK transmembrane domain sequence, a measles F-protein transmembrane domain sequence, a rubella E1 protein transmembrane domain sequence, a rubella E2 protein transmembrane domain sequence, a mumps F-protein transmembrane domain sequence, an Ebola GP protein transmembrane domain sequence and a rabies transmembrane domain sequence, e.g. wherein the transmembrane domain comprises an amino acid sequence selected from the group consisting of sequences set forth by SEQ ID NO: 208-209 and 241-253; and/or
(C) the modified C. acnes CAMP2 polypeptide comprises a sequence according to any one of SEQ ID NO: 207 or SEQ ID NO: 5-9, or a sequence having at least 60% identity thereto.

9. (canceled)

10. A composition comprising:
(a) the nucleic acid of claim 1, optionally wherein the composition is an immunogenic composition;
(b) a nucleic acid comprising a nucleotide sequence encoding a C. acnes CAMP2 polypeptide, wherein the nucleic acid is a mRNA, and wherein the composition is an immunogenic composition; or
(c) a nucleic acid comprising a nucleotide sequence encoding a C. acnes CAMP2 polypeptide and one or more of:
(i) a nucleic acid comprising a nucleotide sequence encoding a C. acnes dermatan sulfate-adhesin 1 (DsA1) polypeptide;
(ii) a nucleic acid comprising a nucleotide sequence encoding a C. acnes dermatan sulfate-adhesin 2 (DsA2) polypeptide;
(iii) a nucleic acid comprising a nucleotide sequence encoding a C. acnes putative iron-transport protein (PITP) polypeptide;
(iv) a nucleic acid comprising a nucleotide sequence encoding a chimeric C. acnes DsA1/DsA2 polypeptide; and
(v) a nucleic acid comprising a nucleotide sequence encoding a chimeric C. acnes DsA1/DsA2/PITP polypeptide;
optionally wherein the composition according to (c) is an immunogenic composition, further optionally wherein the composition according to any one of (a)-(c) is in a frozen liquid form or in a lyophilized form (e.g., in a lyophilized form).

11. The composition of claim 10 (c), wherein the composition comprises the nucleic acid according to (c) (iii) and/or the nucleic acid according to (c) (iv), optionally wherein:
(A) the C. acnes CAMP2 polypeptide comprises a sequence according to SEQ ID NO: 203 or a sequence having at least 60% (e.g., at least 85%) identity thereto;
(B) the chimeric C. acnes DsA1/DsA2 polypeptide comprises a sequence according to SEQ ID NO: 70 or a sequence having at least 90% identity thereto; and
(C) the C. acnes PITP polypeptide comprises a sequence according to SEQ ID NO: 73 or a sequence having at least 75% identity thereto.

12. The composition of claim 10, wherein the composition is as defined in claim 10 (a) or (b), and wherein the composition further comprises one or more of:
(i) a nucleic acid comprising a nucleotide sequence encoding a C. acnes DsA1 polypeptide;
(ii) a nucleic acid comprising a nucleotide sequence encoding a C. acnes DsA2 polypeptide;
(iii) a nucleic acid comprising a nucleotide sequence encoding a C. acnes PITP polypeptide;
(iv) a nucleic acid comprising a nucleotide sequence encoding a chimeric C. acnes DsA1/DsA2 polypeptide; and
(v) a nucleic acid comprising a nucleotide sequence encoding a chimeric C. acnes DsA1/DsA2/PITP polypeptide, optionally wherein:
the composition comprises the nucleic acid according to (iii) and/or the nucleic acid according to (iv), optionally wherein:
(A) the C. acnes CAMP2 polypeptide comprises a sequence according to SEQ ID NO: 207 or a sequence having at least 60% identity thereto; the chimeric C. acnes DsA1/DsA2 polypeptide comprises a sequence according to SEQ ID NO: 70 or a sequence having at least 90% identity thereto; and the C. acnes PITP polypeptide comprises a sequence according to SEQ ID NO: 73 or a sequence having at least 75% identity thereto; and/or
(B) the nucleotide sequence encoding the C. acnes CAMP2 polypeptide comprises a sequence according to SEQ ID NO: 91 or a sequence having at least 50% identity thereto; the nucleotide sequence encoding the chimeric C. acnes DsA1/DsA2 polypeptide comprises a sequence according to SEQ ID NO: 113 or a sequence having at least 75% identity thereto; and the nucleotide sequence encoding the C. acnes PITP polypeptide comprises a sequence according to SEQ ID NO: 115 or a sequence having at least 50% identity thereto.

13-15. (canceled)

16. The composition of claim 10, wherein the composition comprises:
(A) a first mRNA comprising or consisting of the following structural elements:
(i) a 5′ cap;
(ii) a 5′ UTR having the nucleic acid sequence according to SEQ ID NO: 265;
(iii) a protein coding region having the nucleic acid sequence according to SEQ ID NO: 91;
(iv) a 3′ UTR having the nucleic acid sequence according to SEQ ID NO: 266; and
(v) a polyA tail, optionally wherein the polyA tail comprises at least 75 adenosine nucleotides or at least 100 adenosine nucleotides;
(B) a second mRNA comprising or consisting of the following structural elements:
(i) a 5′ cap;
(ii) a 5′ UTR having the nucleic acid sequence according to SEQ ID NO: 265;
(iii) a protein coding region having the nucleic acid sequence according to SEQ ID NO: 113;
(iv) a 3′ UTR having the nucleic acid sequence according to SEQ ID NO: 266; and
(v) a polyA tail, optionally wherein the polyA tail comprises at least 75 adenosine nucleotides or at least 100 adenosine nucleotides; and
(C) a third mRNA comprising or consisting of the following structural elements:
(i) a 5′ cap;
(ii) a 5′ UTR having the nucleic acid sequence according to SEQ ID NO: 265;
(iii) a protein coding region having the nucleic acid sequence according to SEQ ID NO: 115;
(iv) a 3′ UTR having the nucleic acid sequence according to SEQ ID NO: 266; and
(v) a polyA tail, optionally wherein the polyA tail comprises at least 75 adenosine nucleotides or at least 100 adenosine nucleotides;
wherein the 5′ cap has the following structure: 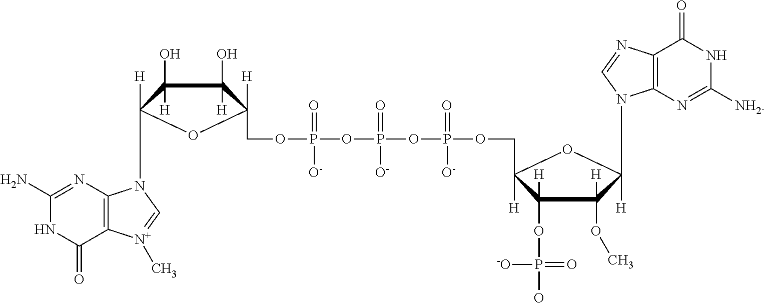
and optionally wherein one or more of the first, second and third mRNAs comprises at least one chemical modification.

17. (canceled)

18. The composition of claim 10, wherein the composition further comprises a lipid nanoparticle (LNP), optionally wherein:
(a) any one or more nucleic acids are encapsulated in the LNP;
(b) any two or more nucleic acids are co-encapsulated in a single LNP; and/or
(c) any two or more nucleic acids are encapsulated in separate LNPs, optionally wherein:
the LNP comprises at least one cationic lipid and wherein the cationic lipid is selected from the group consisting of OF-02, cKK-E10, IM-001, IS-001 and GL-HEPES-E3-E12-DS-4-E10, preferably wherein the cationic lipid is GL-HEPES-E3-E12-DS-4-E10; and/or
the LNP further comprises a polyethylene glycol (PEG) conjugated (PEGylated) lipid, a cholesterol-based lipid, and a helper lipid; and/or
the LNP comprises GL-HEPES-E3-E12-DS-4-E10 at a molar ratio of 35% to 55%;
DMG-PEG2000 at a molar ratio of 0.25% to 2.75%; cholesterol at a molar ratio of 20% to 50%; and DOPE at a molar ratio of 5% to 35%; and/or
the LNP comprises GL-HEPES-E3-E12-DS-4-E10 at a molar ratio of 40%, DMG-PEG2000 at a molar ratio of 1.5%, cholesterol at a molar ratio of 28.5% and DOPE at a molar ratio of 30%.

19-22. (canceled)

23. A composition, optionally an immunogenic composition, comprising:
the modified C. acnes CAMP2 polypeptide of claim 7.

24. The composition of claim 23, wherein the composition further comprises one or more of:
(A) a C. acnes DsA1 polypeptide,
(B) a C. acnes DsA2 polypeptide,
(C) a C. acnes PITP polypeptide,
(D) a chimeric C. acnes DsA1/DsA2 polypeptide, and
(E) a chimeric C. acnes DsA1/DsA2/PITP polypeptide.

25. The composition of claim 10, wherein the C. acnes CAMP2 polypeptide, the C. acnes DsA1 polypeptide, the C. acnes DsA2 polypeptide, the C. acnes PITP polypeptide, the chimeric C. acnes DsA1/DsA2 polypeptide, and the chimeric C. acnes DsA1/DsA2/PITP polypeptide further comprise a non-native transmembrane domain sequence and/or a non-native secretion signal peptide sequence.

26. The composition of claim 10, wherein:
(a) one or more of the C. acnes CAMP2 polypeptide, the C. acnes DsA1 polypeptide, the C. acnes DsA2 polypeptide, the C. acnes PITP polypeptide, the chimeric C. acnes DsA1/DsA2 polypeptide and the chimeric C. acnes DsA1/DsA2/PITP polypeptide comprises a mutation at one or more (e.g., all) positions corresponding to an N-glycosylation site and/or one or more (e.g., all) positions corresponding to an O-glycosylation site in the respective native C. acnes polypeptide, optionally wherein the mutation is a single amino acid substitution; and/or
(b) one or more of the C. acnes DsA1 polypeptide, the C. acnes DsA2 polypeptide, the C. acnes PITP polypeptide, the chimeric C. acnes DsA1/DsA2 polypeptide and the chimeric C. acnes DsA1/DsA2/PITP polypeptide comprises a single amino acid substitution at one or more (e.g., all) positions corresponding to a cysteine residue in the respective native C. acnes polypeptide, optionally wherein the single amino acid substitution is a substitution of cysteine with serine.

27. The composition of claim 10, wherein:
(a) the C. acnes CAMP2 polypeptide comprises a sequence according to any one of SEQ ID NO: 203, SEQ ID NO: 43-58, SEQ ID NO: 1-4, SEQ ID NO: 10-16 or SEQ ID NO: 339-363 or a sequence having at least 60% identity thereto;
(b) the C. acnes DsA1 polypeptide comprises a sequence according to any one of SEQ ID NO: 204 or SEQ ID NO: 17-19 or SEQ ID NO: 59-61, or a sequence having at least 75% identity thereto;
(c) the C. acnes DsA2 polypeptide comprises a sequence according to any one of SEQ ID NO: 205 or SEQ ID NO: 20-27 or SEQ ID NO: 62-69 or a sequence having at least 75% identity thereto;
(d) the C. acnes PITP polypeptide comprises a sequence according to any one of SEQ ID NO: 206, SEQ ID NO: 31-37, SEQ ID NO: 73-79 or a sequence having at least 75% (e.g. 90 or 95%) identity thereto;
(e) the chimeric C. acnes DsA1/DsA2 polypeptide comprises a sequence according to any one of SEQ ID NO: 28-30, SEQ ID NO: 39 or SEQ ID NO: 70-72 or SEQ ID NO: 81, or a sequence having at least 90% identity thereto; and/or
(f) the chimeric C. acnes DsA1/DsA2/PITP polypeptide comprises (i) a sequence of any one of SEQ ID NO: 28-30, SEQ ID NO: 39 or SEQ ID NO: 70-72 or SEQ ID NO: 81, or a sequence having at least 90% identity thereto; and (ii) a PITP polypeptide sequence, optionally wherein the chimeric C. acnes DsA1/DsA2/PITP polypeptide comprises a sequence according to any one of SEQ ID NO: 38, SEQ ID NO: 40-41, SEQ ID NO: 80, SEQ ID NO: 82-83, SEQ ID NO: 367-368, or a sequence having at least 90% identity thereto.

28. The composition of claim 10, wherein the composition comprises:
(a) a C. acnes CAMP2 polypeptide comprising or consisting of a sequence according to SEQ ID NO: 203;
(b) a chimeric C. acnes DsA1/DsA2 polypeptide comprising or consisting of a sequence according to SEQ ID NO: 70; and
(c) C. acnes PITP polypeptide comprising or consisting of a sequence according to SEQ ID NO: 73.

29. The composition of claim 23, wherein the composition further comprises an adjuvant, optionally wherein the adjuvant is selected from the group consisting of: aluminum based adjuvant, squalene based oil in water emulsion adjuvants and liposome-based adjuvants comprising a saponin and a TLR4 agonist, further optionally wherein the adjuvant is selected from the group consisting of AlOOH, AF03 and SPA14.

30. A nucleic acid comprising a nucleotide sequence encoding a chimeric C. acnes DsA1/DsA2/PITP/CAMP2 polypeptide, wherein the chimeric C. acnes DsA1/DsA2/PITP/CAMP2 polypeptide comprises:
(a) a chimeric C. acnes DsA1/DsA2 polypeptide;
(b) an immunogenic fragment of a C. acnes PITP polypeptide, optionally wherein the immunogenic fragment comprises a ENFD of a C. acnes PITP polypeptide; and
(c) a C. acnes CAMP2 polypeptide or an immunogenic fragment thereof.

31. A chimeric C. acnes DsA1/DsA2/PITP/CAMP2 polypeptide comprising:
(a) a chimeric C. acnes DsA1/DsA2 polypeptide;
(b) an immunogenic fragment of a C. acnes PITP polypeptide, optionally wherein the immunogenic fragment comprises a ENFD of a C. acnes PITP polypeptide; and
(c) a C. acnes CAMP2 polypeptide or an immunogenic fragment thereof.

32. The nucleic acid of claim 30, wherein
i. the chimeric C. acnes DsA1/DsA2/PITP/CAMP2 polypeptide comprises a first conserved sub-domain (CSD1) of a C. acnes DsA1 polypeptide, a second conserved sub-domain (CSD2) of a C. acnes DsA2 polypeptide and a third conserved sub-domain (CSD3) of a C. acnes DsA1 polypeptide, optionally wherein the chimeric C. acnes DsA1/DsA2 polypeptide of (a) comprises a sequence according to SEQ ID NO: 70, or a sequence having at least 90%, identity thereto;
ii. the immunogenic fragment of a C. acnes PITP polypeptide in (b) comprising an extended neocarzinostatin family domain (ENFD) of a C. acnes PITP polypeptide comprises the sequence corresponding to amino acid residues 1-133 or residues 1-146 of SEQ ID NO: 73 or a sequence having at least 90% identity thereto; and/or
iii. (c) is (1) a C. acnes CAMP2 polypeptide, optionally wherein (c) comprises SEQ ID NO: 203 or a sequence having at least 90% identity thereto; or (2) an immunogenic fragment of a C. acnes CAMP2 polypeptide comprising a N-terminal domain of a C. acnes CAMP2 polypeptide, e.g. wherein the immunogenic fragment comprises amino acid residues 29-176 of SEQ ID NO: 202 or a sequence having at least 90% identity to the sequence of amino acid residues 29-176 of SEQ ID NO: 202.

33. (canceled)

34. The nucleic acid of claim 30, wherein:
(i) the chimeric C. acnes DsA1/DsA2/PITP/CAMP2 polypeptide comprises a sequence according to SEQ ID NO: 374-375; or
(ii) the chimeric C. acnes DsA1/DsA2/PITP/CAMP2 polypeptide comprises a sequence according to SEQ ID NO: 373 and a transmembrane domain sequence
optionally wherein the chimeric C. acnes DsA1/DsA2/PITP/CAMP2 polypeptide comprises a sequence according to SEQ ID NO: 374 or wherein the chimeric C. acnes DsA1/DsA2/PITP/CAMP2 polypeptide comprises a sequence according to SEQ ID NO: 373 and a TMB sequence according to SEQ ID NO: 84; and/or
(i) the nucleotide sequence encoding the chimeric C. acnes DsA1/DsA2/PITP/CAMP2 polypeptide comprises a sequence according to any one of SEQ ID NO: 377-382 or 384-389; or
(ii) the nucleotide sequence encoding the chimeric C. acnes DsA1/DsA2/PITP/CAMP2 polypeptide comprises a sequence according to SEQ ID NO: 384 and a sequence according to SEQ ID NO: 395; or a sequence according to SEQ ID NO: 385 and a sequence according to SEQ ID NO: 396,
optionally wherein the nucleotide sequence encoding the chimeric C. acnes DsA1/DsA2/PITP/CAMP2 polypeptide comprises a sequence according to SEQ ID NO: 387 or the nucleotide sequence encoding the chimeric C. acnes DsA1/DsA2/PITP/CAMP2 polypeptide comprises a sequence according to SEQ ID NO: 385 and a sequence according to SEQ ID NO: 396.

35-40. (canceled)

41. A method of generating an immune response against a C. acnes infection in a subject or a method of treating or preventing C. acnes infection in a subject, the method comprising administering the nucleic acid of claim 1 to the subject.

42. (canceled)

43. The method of claim 41, wherein the method comprises:
(i) administering one dose of the nucleic acid, the polypeptide or the composition; or
(ii) administering two doses of the nucleic acid, the polypeptide or the composition, optionally wherein the two doses are administered two months apart; and/or
the subject is a human subject and wherein the subject is between 9 and 45 years old or between 12 and 45 years old; and/or
the method comprises administering the nucleic acid intramuscularly or through skin injection.

44. (canceled)

45. (canceled)

46. A method of generating an immune response against a C. acnes infection in a subject or a method of treating or preventing C. acnes infection in a subject, the method comprising administering the modified C. acnes CAMP2 polypeptide of claim 7 to the subject.

47. A method of generating an immune response against a C. acnes infection in a subject or a method of treating or preventing C. acnes infection in a subject, the method comprising administering the composition of claim 10 to the subject.

48. A method of generating an immune response against a C. acnes infection in a subject or a method of treating or preventing C. acnes infection in a subject, the method comprising administering the composition of claim 23 to the subject.

49. A method of generating an immune response against a C. acnes infection in a subject or a method of treating or preventing C. acnes infection in a subject, the method comprising administering the nucleic acid of claim 30 to the subject.

50. A method of generating an immune response against a C. acnes infection in a subject or a method of treating or preventing C. acnes infection in a subject, the method comprising administering the chimeric C. acnes DsA1/DsA2/PITP/CAMP2 polypeptide of claim 31 to the subject.

51. A composition, optionally an immunogenic composition, comprising a C. acnes CAMP2 polypeptide and one or more of:
(i) a C. acnes DsA1 polypeptide,
(ii) a C. acnes DsA2 polypeptide,
(iii) a C. acnes PITP polypeptide,
(iv) a chimeric C. acnes DsA1/DsA2 polypeptide, and
(v) a chimeric C. acnes DsA1/DsA2/PITP polypeptide.

52. A method of generating an immune response against a C. acnes infection in a subject or a method of treating or preventing C. acnes infection in a subject, the method comprising administering the composition of claim 51 to the subject.


SEQUENCE LISTING (399 sequences)

SEQ ID NO: 1 - protein, 240 aa, synthetic construct
MVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 2 - protein, 256 aa, synthetic construct
MKAKLLVLLCTFTATYAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 3 - protein, 256 aa, synthetic construct; identical to SEQ ID NO: 2

SEQ ID NO: 4 - protein, 256 aa, synthetic construct; identical to SEQ ID NO: 2

SEQ ID NO: 5 - protein, 285 aa, synthetic construct
MKAKLLVLLCTFTATYAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAASGSILAIYSTVASSLVLLVSLGAISFGSG

SEQ ID NO: 6 - protein, 285 aa, synthetic construct; identical to SEQ ID NO: 5

SEQ ID NO: 7 - protein, 271 aa, synthetic construct
MNPNQKIITIGSISIAIGIISLMLQIGNISGSVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 8 - protein, 271 aa, synthetic construct
MNPNQKIITIGSISIAIGIISLMLQIGNISGSVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTSFSKTIWQVRANRDRYILGHKSAAVYKTLNHAITKAVGVRLNPKTTVGNIQAARTELLAAYQTAFNSPDVKKAA

SEQ ID NO: 9 - protein, 285 aa, synthetic construct
MKAKLLVLLCTFTATYAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTSFSKTIWQVRANRDRYILGHKSAAVYKTLNHAITKAVGVRLNPKTTVGNIQAARTELLAAYQTAFNSPDVKKAASGSILAIYSTVASSLVLLVSLGAISFGSG

SEQ ID NO: 10 - protein, 256 aa, synthetic construct; identical to SEQ ID NO: 2

SEQ ID NO: 11 - protein, 256 aa, synthetic construct; identical to SEQ ID NO: 2

SEQ ID NO: 12 - protein, 256 aa, synthetic construct
MKAKLLVLLCTFTATYAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTSFSKTIWQVRANRDRYILGHKSAAVYKTLNHAITKAVGVRLNPKTTVGNIQAARTELLAAYQTAFNSPDVKKAA

SEQ ID NO: 13 - protein, 256 aa, synthetic construct
MKAKLLVLLCTFTATYAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPGGTPNGVGGVYVRTNFSKTIWQVRANRDRYILGHKSAAVYKTLNHAITKAVGVRLNPKTTVGNIQAARTELLAAYQTAFNSPDVKKAA

SEQ ID NO: 14 - protein, 256 aa, synthetic construct
MKAKLLVLLCTFTATYAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPGGTPNGVGGVYVRTSFSKTIWQVRANRDRYILGHKSAAVYKTLNHAITKAVGVRLNPKTTVGNIQAARTELLAAYQTAFNSPDVKKAA

SEQ ID NO: 15 - protein, 256 aa, synthetic construct
MKAKLLVLLCTFTATYAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVAANADRYIAGHKSAAVAKTLAHAIAKAAGVRANPKTTVGNIQAARTELLAAYQTAFNSPDVKKAA

SEQ ID NO: 16 - protein, 256 aa, synthetic construct
MKAKLLVLLCTFTATYAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTSFSKTIWQVAANADRYIAGHKSAAVAKTLAHAIAKAAGVRANPKTTVGNIQAARTELLAAYQTAFNSPDVKKAA

SEQ ID NO: 17 - protein, 342 aa, synthetic construct
MKAKLLVLLCTFTATYASSNRPRSVAQAAIATDGKGIINKDCRDAVINDAKLRAAIAGALVKAGFSSADAVALAPRIAREMAKEGVLLINHHKLKALIGAQVGLLTDAKIQRAAAAVDLGIKATLAATIIPNALHSAAFKDAVVANLVAAGVDKKLAKATAVAIAATALNPALGPIAKTEAIKAEIGAQAALLVSRGVHLKKAAIEHIIGRSFDAAVATAIVSSPILNARIVTHLVRAGIDKSLAVQIAPRIIDRLAKEPLLALNTAKLMKNITRQIVDVITADKAIKTAEQLEKELPALDDLVKKACSSPKPTPTILAIYSTVASSLVLLVSLGAISFGSG

SEQ ID NO: 18 - protein, 309 aa, synthetic construct
MKAKLLVLLCTFTATYASSNRPRSVAQAAIATDGKGIINKDCRDAVINDAKLRAAIAGALVKAGFSSADAVALAPRIAREMAKEGVLLINHHKLKALIGAQVGLLTDAKIQRAAAAVDLGIKATLAATIIPNALHSAAFKDAVVANLVAAGVDKKLAKATAVAIAATALNPALGPIAKTEAIKAEIGAQAALLVSRGVHLKKAAIEHIIGRSFDAAVATAIVSSPILNARIVTHLVRAGIDKSLAVQIAPRIIDRLAKEPLLALNTAKLMKNITRQIVDVITADKAIKTAEQLEKELPALDDLVKKACS

SEQ ID NO: 19 - protein, 338 aa, synthetic construct
MKAKLLVLLCTFTATYASSNRPRSVAQAAIATDGKGIINKDCRDAVINDAKLRAAIAGALVKAGFSSADAVALAPRIAREMAKEGVLLINHHKLKALIGAQVGLLTDAKIQRAAAAVDLGIKATLAATIIPNALHSAAFKDAVVANLVAAGVDKKLAKATAVAIAATALNPALGPIAKTEAIKAEIGAQAALLVSRGVHLKKAAIEHIIGRSFDAAVATAIVSSPILNARIVTHLVRAGIDKSLAVQIAPRIIDRLAKEPLLALNTAKLMKNITRQIVDVITADKAIKTAEQLEKELPALDDLVKKACSSGSILAIYSTVASSLVLLVSLGAISFGSG

SEQ ID NO: 20 - protein, 342 aa, synthetic construct
MKAKLLVLLCTFTATYAASNGNSSITQSAAFSPRATTKISEDCRKAIINDLKLRGAIVGALVKAGLSAADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACTPEPTPTILAIYSTVASSLVLLVSLGAISFGSG

SEQ ID NO: 21 - protein, 339 aa, synthetic construct
MKAKLLVLLCTFTATYAASNGNSSITQSAAFSPRATTKISEDCRKAIINDLKLRGAIVGALVKAGLSAADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACTSGSILAIYSTVASSLVLLVSLGAISFGSG

SEQ ID NO: 22 - protein, 310 aa, synthetic construct
MKAKLLVLLCTFTATYAASNGNSSITQSAAFSPRATTKISEDCRKAIINDLKLRGAIVGALVKAGLSAADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACT

SEQ ID NO: 23 - protein, 310 aa, synthetic construct
MKAKLLVLLCTFTATYAASNGNSSITQSAAFSPRATTKISEDCRKAIINDLKLRGAIVGALVKAGLSVADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACT

SEQ ID NO: 24 - protein, 342 aa, synthetic construct
MKAKLLVLLCTFTATYAASNGNSSITQSAAFSPRATTKISEDCRKAIINDLKLRGAIVGALVKAGLSVADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACTPEPTPTILAIYSTVASSLVLLVSLGAISFGSG

SEQ ID NO: 25 - protein, 339 aa, synthetic construct
MKAKLLVLLCTFTATYAASNGNSSITQSAAFSPRATTKISEDCRKAIINDLKLRGAIVGALVKAGLSVADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACTSGSILAIYSTVASSLVLLVSLGAISFGSG

SEQ ID NO: 26 - protein, 339 aa, synthetic construct
MKAKLLVLLCTFTATYAASNGQSSITQSAAFSPRATTKISEDCRKAIINDLKLRGAIVGALVKAGLSAADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACTSGSILAIYSTVASSLVLLVSLGAISFGSG

SEQ ID NO: 27 - protein, 342 aa, synthetic construct
MKAKLLVLLCTFTATYAASNGQSSITQSAAFSPRATTKISEDCRKAIINDLKLRGAIVGALVKAGLSAADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACTPEPTPTILAIYSTVASSLVLLVSLGAISFGSG

SEQ ID NO: 28 - protein, 322 aa, synthetic construct
MKAKLLVLLCTFTATYASSNRPRSVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKASSPPKPTPTPTPTPT

SEQ ID NO: 29 - protein, 306 aa, synthetic construct
MSSNRPRSVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKASSPPKPTPTPTPTPT

SEQ ID NO: 30 - protein, 322 aa, synthetic construct
MKAKLLVLLCTFTATYASSNRPRSVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKQITRQIVDVITADKAIKTAEQLEKELPALDDLVKKASSPPKPTPTPTPTPT

SEQ ID NO: 31 - protein, 416 aa, synthetic construct
MKAKLLVLLCTFTATYAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVSTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVSHNVTVDAVTGKVIGDDSGKGAGRGLPVT

SEQ ID NO: 32 - protein, 400 aa, synthetic construct
MAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVSTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVSHNVTVDAVTGKVIGDDSGKGAGRGLPVT

SEQ ID NO: 33 - protein, 416 aa, synthetic construct
MKAKLLVLLCTFTATYAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 34 - protein, 416 aa, synthetic construct
MKAKLLVLLCTFTATYAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIAKGSWKLSGGANWNGGAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTQLTLSAEKVCHSVTVDAVTGKVIGDDSGKGAGRGLPVT

SEQ ID NO: 35 - protein, 416 aa, synthetic construct
MKAKLLVLLCTFTATYAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVSTIGASKVTSGSLTWGIRTSFTSYLRGPIAKGSWKLSGGANWNGGAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTQLTLSAEKVSHSVTVDAVTGKVIGDDSGKGAGRGLPVT

SEQ ID NO: 36 - protein, 445 aa, synthetic construct
MKAKLLVLLCTFTATYAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVTSGSILAIYSTVASSLVLLVSLGAISFGSG

SEQ ID NO: 37 - protein, 445 aa, synthetic construct
MKAKLLVLLCTFTATYAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIAKGSWKLSGGANWNGGAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTQLTLSAEKVCHSVTVDAVTGKVIGDDSGKGAGRGLPVTSGSILAIYSTVASSLVLLVSLGAISFGSG

SEQ ID NO: 38 - protein, 468 aa, synthetic construct
MKAKLLVLLCTFTATYASSNRPRSVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKASSPPKPTPTPTPTPTAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPI

SEQ ID NO: 39 - protein, 307 aa, synthetic construct
MKAKLLVLLCTFTATYAVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKAITRQIVDVITADKAIKTAEQLEKELPALDDLVKKAMGPPKPT

SEQ ID NO: 40 - protein, 452 aa, synthetic construct
MKAKLLVLLCTFTATYASSNRPRSVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKAMGPPKPTGGGGGAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKGDHGDDTRTPVGYR

SEQ ID NO: 41 - protein, 455 aa, synthetic construct
MKAKLLVLLCTFTATYASSNRPRSVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKASSPPKPTPTPTPTPTAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYR

SEQ ID NO: 42 - protein, 17 aa, synthetic construct
MKAKLLVLLCTFTATYA

SEQ ID NO: 43 - protein, 240 aa, synthetic construct; identical to SEQ ID NO: 1

SEQ ID NO: 44 - protein, 239 aa, synthetic construct
VEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 45 - protein, 239 aa, synthetic construct; identical to SEQ ID NO: 44

SEQ ID NO: 46 - protein, 239 aa, synthetic construct; identical to SEQ ID NO: 44

SEQ ID NO: 47 - protein, 239 aa, synthetic construct; identical to SEQ ID NO: 44

SEQ ID NO: 48 - protein, 239 aa, synthetic construct; identical to SEQ ID NO: 44

SEQ ID NO: 49 - protein, 239 aa, synthetic construct; identical to SEQ ID NO: 44

SEQ ID NO: 50 - protein, 239 aa, synthetic construct
VEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTSFSKTIWQVRANRDRYILGHKSAAVYKTLNHAITKAVGVRLNPKTTVGNIQAARTELLAAYQTAFNSPDVKKAA

SEQ ID NO: 51 - protein, 239 aa, synthetic construct; identical to SEQ ID NO: 50

SEQ ID NO: 52 - protein, 239 aa, synthetic construct; identical to SEQ ID NO: 44

SEQ ID NO: 53 - protein, 239 aa, synthetic construct; identical to SEQ ID NO: 44

SEQ ID NO: 54 - protein, 239 aa, synthetic construct; identical to SEQ ID NO: 50

SEQ ID NO: 55 - protein, 239 aa, synthetic construct
VEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPGGTPNGVGGVYVRTNFSKTIWQVRANRDRYILGHKSAAVYKTLNHAITKAVGVRLNPKTTVGNIQAARTELLAAYQTAFNSPDVKKAA

SEQ ID NO: 56 - protein, 239 aa, synthetic construct
VEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPGGTPNGVGGVYVRTSFSKTIWQVRANRDRYILGHKSAAVYKTLNHAITKAVGVRLNPKTTVGNIQAARTELLAAYQTAFNSPDVKKAA

SEQ ID NO: 57 - protein, 239 aa, synthetic construct
VEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVAANADRYIAGHKSAAVAKTLAHAIAKAAGVRANPKTTVGNIQAARTELLAAYQTAFNSPDVKKAA

SEQ ID NO: 58 - protein, 239 aa, synthetic construct
VEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTSFSKTIWQVAANADRYIAGHKSAAVAKTLAHAIAKAAGVRANPKTTVGNIQAARTELLAAYQTAFNSPDVKKAA

SEQ ID NO: 59 - protein, 299 aa, synthetic construct
SSNRPRSVAQAAIATDGKGIINKDCRDAVINDAKLRAAIAGALVKAGFSSADAVALAPRIAREMAKEGVLLINHHKLKALIGAQVGLLTDAKIQRAAAAVDLGIKATLAATIIPNALHSAAFKDAVVANLVAAGVDKKLAKATAVAIAATALNPALGPIAKTEAIKAEIGAQAALLVSRGVHLKKAAIEHIIGRSFDAAVATAIVSSPILNARIVTHLVRAGIDKSLAVQIAPRIIDRLAKEPLLALNTAKLMKNITRQIVDVITADKAIKTAEQLEKELPALDDLVKKACSSPKPTPT

SEQ ID NO: 60 - protein, 292 aa, synthetic construct
SSNRPRSVAQAAIATDGKGIINKDCRDAVINDAKLRAAIAGALVKAGFSSADAVALAPRIAREMAKEGVLLINHHKLKALIGAQVGLLTDAKIQRAAAAVDLGIKATLAATIIPNALHSAAFKDAVVANLVAAGVDKKLAKATAVAIAATALNPALGPIAKTEAIKAEIGAQAALLVSRGVHLKKAAIEHIIGRSFDAAVATAIVSSPILNARIVTHLVRAGIDKSLAVQIAPRIIDRLAKEPLLALNTAKLMKNITRQIVDVITADKAIKTAEQLEKELPALDDLVKKACS

SEQ ID NO: 61 - protein, 292 aa, synthetic construct; identical to SEQ ID NO: 60

SEQ ID NO: 62 - protein, 299 aa, synthetic construct
ASNGNSSITQSAAFSPRATTKISEDCRKAIINDLKLRGAIVGALVKAGLSAADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACTPEPTPT

SEQ ID NO: 63 - protein, 293 aa, synthetic construct
ASNGNSSITQSAAFSPRATTKISEDCRKAIINDLKLRGAIVGALVKAGLSAADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACT

SEQ ID NO: 64 - protein, 293 aa, synthetic construct; identical to SEQ ID NO: 63

SEQ ID NO: 65 - protein, 293 aa, synthetic construct
ASNGNSSITQSAAFSPRATTKISEDCRKAIINDLKLRGAIVGALVKAGLSVADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACT

SEQ ID NO: 66 - protein, 299 aa, synthetic construct
ASNGNSSITQSAAFSPRATTKISEDCRKAIINDLKLRGAIVGALVKAGLSVADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACTPEPTPT

SEQ ID NO: 67 - protein, 293 aa, synthetic construct; identical to SEQ ID NO: 65

SEQ ID NO: 68 - protein, 293 aa, synthetic construct
ASNGQSSITQSAAFSPRATTKISEDCRKAIINDLKLRGAIVGALVKAGLSAADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACT

SEQ ID NO: 69 - protein, 299 aa, synthetic construct
ASNGQSSITQSAAFSPRATTKISEDCRKAIINDLKLRGAIVGALVKAGLSAADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACTPEPTPT

SEQ ID NO: 70 - protein, 305 aa, synthetic construct
SSNRPRSVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKASSPPKPTPTPTPTPT

SEQ ID NO: 71 - protein, 306 aa, synthetic construct; identical to SEQ ID NO: 29

SEQ ID NO: 72 - protein, 305 aa, synthetic construct
SSNRPRSVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKQITRQIVDVITADKAIKTAEQLEKELPALDDLVKKASSPPKPTPTPTPTPT

SEQ ID NO: 73 - protein, 399 aa, synthetic construct
AGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVSTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVSHNVTVDAVTGKVIGDDSGKGAGRGLPVT

SEQ ID NO: 74 - protein, 400 aa, synthetic construct; identical to SEQ ID NO: 32

SEQ ID NO: 75 - protein, 399 aa, synthetic construct
AGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 76 - protein, 399 aa, synthetic construct
AGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIAKGSWKLSGGANWNGGAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTQLTLSAEKVCHSVTVDAVTGKVIGDDSGKGAGRGLPVT

SEQ ID NO: 77 - protein, 399 aa, synthetic construct
AGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVSTIGASKVTSGSLTWGIRTSFTSYLRGPIAKGSWKLSGGANWNGGAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTQLTLSAEKVSHSVTVDAVTGKVIGDDSGKGAGRGLPVT

SEQ ID NO: 78 - protein, 399 aa, synthetic construct; identical to SEQ ID NO: 75

SEQ ID NO: 79 - protein, 399 aa, synthetic construct; identical to SEQ ID NO: 76

SEQ ID NO: 80 - protein, 451 aa, synthetic construct
SSNRPRSVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKASSPPKPTPTPTPTPTAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPI

SEQ ID NO: 81 - protein, 290 aa, synthetic construct
VAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKAITRQIVDVITADKAIKTAEQLEKELPALDDLVKKAMGPPKPT

SEQ ID NO: 82 - protein, 435 aa, synthetic construct
SSNRPRSVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKAMGPPKPTGGGGGAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKGDHGDDTRTPVGYR

SEQ ID NO: 83 - protein, 438 aa, synthetic construct
SSNRPRSVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKASSPPKPTPTPTPTPTAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYR

SEQ ID NO: 84 - protein, 29 aa, synthetic construct
SGSILAIYSTVASSLVLLVSLGAISFGSG

SEQ ID NO: 85 - protein, 26 aa, synthetic construct
ILAIYSTVASSLVLLVSLGAISFGSG

SEQ ID NO: 86 - protein, 26 aa, synthetic construct
ILAIYSTVASSLVLLVSLGAISFGSG

SEQ ID NO: 87 - RNA, 771 nt, synthetic construct
  source 1..771: mol_type = other RNA
atgaaggccaagctgctggtgctgctgtgcacctttacagccacctacgccgtggaacccaccaccacaatcagcgccacaagcacacacgagctgagcgcctccgatgccagaaactctatccagctgctgaacgcccacattgccacactgcagagcgtgcagaaaagcgtgccaggcagcgactacagcgaccagatcagagatctgctgaaggccgccttcgacctgagaggcctgattgaaacactggcccacggcggcatcccattctacgatcctagcaccatcatgccccggatcaagctggtggccacaaccatcgataccatccacaccgccaccacaactctgcagaacaaagtgcggcctgctcacgtggaactgggcctcgaagtgacaaaagccgtgctgctgacagccaatcctgccagcacagccaaagagctggacgctgaaggcgccgctctgaaggccagactggaaaaagtgtctcagtaccccgatctgacccctaacgacgtggccacagtgtacgtgcggaccaacttcagcaagaccatctggcaagtgcgggccaacagagatcggtacatcctgggacacaagagcgccgctgtgtacaagaccctgaaccacgccatcacaaaggccgtgggcgtcagactgaaccccaagaccacagtgggcaacatccaggccgccagaacagaactgctggccgcctatcagaccgccttcaacagccccgatgtgaagaaagccgcttaa

SEQ ID NO: 88 - RNA, 771 nt, synthetic construct
  source 1..771: mol_type = other RNA
atgaaagcaaagctgctggtgctgctgtgcaccttcaccgctacctacgccgtcgagcctacaacaacaatcagcgccacaagcacccacgagctgagcgcctctgatgcccggaactctatccagctgctgaacgcccacatcgccaccctgcagagcgtgcagaagtctgtgccaggcagcgactacagcgaccagatcagagatctgcttaaagccgctttcgacctgagaggcctgatcgagacactggcccacggcggaatccccttctacgaccccagcaccatcatgcctagaatcaagctggtcgccaccaccatcgacaccatccacaccgccacaaccaccctgcagaacaaggtgcggcctgcccatgtggaactgggactggaggtgacaaaagccgtgctgctcaccgccaacccagctagcacagccaaggaactggacgccgagggcgccgccctgaaggccagactggaaaaggtgtcccagtaccccgacctgacccctaacgacgtggccaccgtgtacgtgcgcaccaacttcagcaagaccatttggcaggtgcgggccaatagagatagatacatcctgggccacaagtccgctgccgtgtacaagacactgaatcacgccatcaccaaggctgttggcgtgcggctgaaccctaagaccactgtgggcaacatccaggccgctagaacagagctgctggccgcctatcaaacagcttttaacagccctgatgtgaagaaagccgcttaa

SEQ ID NO: 89 - RNA, 771 nt, synthetic construct
  source 1..771: mol_type = other RNA
atgaaggccaagctgctggtgctgctctgcaccttcaccgccacctacgccgtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacaccaaacgatgtggccactgtgtacgtgagaaccaacttctccaagaccatctggcaggtcagagccaaccgcgatcgctacatcctgggccacaagtccgccgccgtgtataagaccctgaaccatgccatcaccaaggccgtgggcgtcagactgaaccccaagaccaccgtgggcaacatccaggccgctagaacagagctgctggccgcctaccagaccgccttcaattcacccgacgtgaagaaggccgcttaa

SEQ ID NO: 90 - RNA, 858 nt, synthetic construct
  source 1..858: mol_type = other RNA
atgaaggccaagctgctggtgctgctgtgcaccttcaccgccacctatgccgtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacccccaacgacgtggccactgtgtacgtgcggaccaacttcagcaagaccatttggcaggtcagggccaatagagacagatacatcctggggcacaaaagcgccgccgtgtacaagaccctgaaccacgccatcaccaaggccgtgggagtgcggctgaatccaaagaccaccgtgggcaacattcaggccgccagaaccgagctgctggccgcctaccagacagctttcaatagccccgacgtgaagaaggccgcctccggcagcatcctggccatctactccaccgtggcctccagcctggtgctgctggtgagcctgggcgctatctccttcgggtctgggtaa

SEQ ID NO: 91 - RNA, 861 nt, synthetic construct
  source 1..861: mol_type = other RNA
atgaaagccaagctgctggtgctgctctgcacttttaccgccacctacgccgtggagcccaccacaacaatcagcgccacatccacccacgaactgagcgcctccgacgccagaaattccatccagctgctgaacgcccacatcgccaccctgcagagcgtgcagaaaagcgtgcccggctctgattacagcgaccagatccgggacctgctgaaggccgcctttgacctgagaggcctgatcgagacactggcccacggaggcatccctttctacgatcctagcaccatcatgcctcggatcaagctggtggccacaacaatcgacaccatccacaccgccaccaccacactgcagaacaaagtgcggcctgcccatgtggaactgggcctggaagtgaccaaggccgtgctgctgacagccaatcctgctagcacagccaaggaactggatgccgagggagccgccctgaaggctagactggagaaggtgagccagtaccccgacctgacccccaatgacgtggcaaccgtgtacgtgagaaccaatttcagcaagaccatctggcaggtgcgggccaatcgggacagatacatcctgggccacaagagcgccgccgtgtacaagaccctgaatcacgccatcaccaaggccgtgggcgtgcggctgaatcctaagaccaccgtgggcaatatccaggccgcccggacagaactgctggccgcctaccagacagcctttaatagccctgatgtgaagaaggccgccagcgggagcattctggccatctactctaccgtggcctctagcctggtgctgctggtgtctctgggagccattagcttcggatctggctgataa

SEQ ID NO: 92 - RNA, 816 nt, synthetic construct
  source 1..816: mol_type = other RNA
atgaaccccaaccagaaaatcatcaccatcggctctatttctatcgccatcggaatcatctccctgatgctgcagatcggcaacatcagcggcagcgtggaacctaccacaaccatcagcgccaccagcacacacgagctgagcgcttctgatgcccggaacagcatccaactgctgaatgcccacatcgccacactccagagcgtccagaagagcgtccccggcagcgactacagcgatcagatcagagacctgctgaaggccgctttcgacctgagaggcctgatcgagacacttgcccacggcggcatccccttctacgaccccagcaccatcatgcctagaatcaagctggtggccaccaccatcgacaccatccacaccgccacaactaccctgcaaaacaaggtgcgcccagcccatgtggaactgggcctggaggtgaccaaggcagttctgctgacagctaatcctgcctctaccgctaaagagctggacgccgagggcgccgccctgaaagccagactggaaaaggtgtcccagtaccctgatctgacccctaacgacgtggccacagtgtacgtgcggaccaacttcagcaagaccatctggcaggtgcgggccaacagagatagatacatcctgggacacaagtccgccgccgtgtacaagaccctgaaccacgccattaccaaagccgtgggcgtgcggctgaaccctaagacaaccgtgggaaatatccaggccgctagaaccgagctgctggccgcttatcagacagcctttaacagccctgacgtgaagaaggccgcctga

SEQ ID NO: 93 - RNA, 816 nt, synthetic construct
  source 1..816: mol_type = other RNA
atgaaccccaaccagaagatcattaccatcggcagcatcagcatcgccatcggcatcatcagtctgatgctgcagattggcaacatctccggcagcgtcgagcctaccacaaccatcagcgccacatctacacacgagctgagcgccagcgacgccagaaacagcatccagctgctgaacgcccacatcgccacactgcagagcgtgcagaaatctgtgcccggctccgattacagcgatcagatcagagacctgcttaaagccgctttcgacctgagaggactgatcgagacactggcccacggaggcatccccttctacgaccccagcaccatcatgcctagaatcaagctggtggccaccaccatcgacactatccacaccgctaccacaaccctgcaaaacaaggtgcggcctgcccatgtggaactgggcctggaagtgaccaaagccgtgctgctgaccgccaaccctgcttctaccgccaaggaactggacgccgagggcgccgctctgaaagcgcggctggagaaggtttctcagtaccctgatctgacccctaacgacgtggccacagtgtacgtgaggaccagcttcagcaagaccatctggcaggtgcgggccaatagagatagatacatcctgggccacaagtccgccgccgtgtataagacactgaaccacgccatcaccaaggctgtcggagtgcggctgaatccaaagaccaccgtgggcaatatccaagccgctagaaccgagctgctcgccgcctaccagacagcttttaacagccctgacgtgaagaaggccgcctga

SEQ ID NO: 94 - RNA, 858 nt, synthetic construct
  source 1..858: mol_type = other RNA
atgaaggccaagctgctggtgctgctgtgcaccttcaccgccacatacgccgtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacccccaacgatgtggccaccgtgtatgtgcggacctccttctccaagaccatctggcaggtcagagccaatcgggaccggtacatcctgggccacaagagcgccgctgtgtacaagaccctgaaccatgccatcaccaaggcagtgggcgtgcgcctgaacccaaaaaccaccgtgggcaacattcaggccgccagaaccgagctgctggccgcctaccagaccgctttcaatagccccgacgtgaagaaggccgccagcggctccatcctggccatctactctaccgtggccagcagcctggtgctgctggtctccctgggagccatctccttcggctccgggtaa

SEQ ID NO: 95 - RNA, 771 nt, synthetic construct; identical to SEQ ID NO: 87
  source 1..771: mol_type = other RNA

SEQ ID NO: 96 - RNA, 771 nt, synthetic construct; identical to SEQ ID NO: 88
  source 1..771: mol_type = other RNA

SEQ ID NO: 97 - RNA, 771 nt, synthetic construct
  source 1..771: mol_type = other RNA
atgaaggccaagctgctggtgctgctgtgcaccttcaccgccacctacgccgtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgaccccaaatgacgtggccaccgtgtacgtgcggacctccttctccaagaccatctggcaagtgcgggctaacagagacaggtacattctgggccacaagtccgccgccgtgtacaaaaccctgaatcacgccatcaccaaggccgtgggcgtgagactgaaccccaagaccaccgtgggcaacattcaggctgccaggaccgagctgctggccgcttatcagaccgccttcaacagccccgacgtgaagaaagccgcctaa

SEQ ID NO: 98 - RNA, 771 nt, synthetic construct
  source 1..771: mol_type = other RNA
atgaaggcaaagctgctggtgctgctgtgcaccttcaccgccacatacgcagtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccggcgggacacctaacggcgtgggaggcgtgtatgtgaggacaaacttcagcaagacaatctggcaggtcagggccaaccgcgatagatacatcctgggccacaagagcgccgccgtctacaagacactgaaccacgctatcacaaaagccgtgggggtgaggctgaaccccaagaccaccgtggggaacatccaggccgctagaactgagctgctggccgcctaccagaccgctttcaatagccccgacgtgaagaaggccgcctaa

SEQ ID NO: 99 - RNA, 771 nt, synthetic construct
  source 1..771: mol_type = other RNA
atgaaagccaagctgctggtgctgctgtgcaccttcaccgccacatacgccgtggaacctacaaccacaatcagcgccacatctacacacgagctgagcgccagcgacgcccggaacagcatccagctgctgaacgcccacatcgccaccctccagagcgtgcagaaatccgtgcctggatctgattacagcgaccagatcagagatctgctgaaggccgcttttgacctgagaggcctgatcgagacactggcccatggcggcatccccttctacgaccctagcacaattatgcctagaatcaagctggtcgccaccaccatcgacaccatccacaccgccaccacaaccctgcagaacaaggtcagaccagcccacgtggaactcggcctggaggtgaccaaggctgtgctgcttacagctaatcctgctagcaccgctaaagagctggacgccgagggcgccgccctgaaggcccgcctggaaaaagtgtcccagtaccccggcggaacccctaacggcgttggcggagtgtacgtgcggaccagcttcagcaagaccatctggcaggtgcgggctaatagagatagatacatcctgggccacaagtccgccgcggtgtataagacactgaaccacgccatcaccaaggctgtgggcgtgcggctgaaccccaagaccacagtgggcaacatccaagccgccagaaccgagctgctggccgcctaccagaccgccttcaactctccagacgtgaagaaggccgcctga

SEQ ID NO: 100 - RNA, 771 nt, synthetic construct
  source 1..771: mol_type = other RNA
atgaaggccaagctgctggtgctgctgtgcaccttcacagccacctacgccgtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacccccaacgatgtggctaccgtctatgtgcggactaatttcagtaagaccatctggcaggtcgccgccaacgccgatagatatatcgccggccacaaatccgccgccgtcgcaaagacccttgctcacgctattgccaaggccgctggcgtgagagctaacccaaagaccaccgtcggcaacatccaggccgccaggacagagctgcttgccgcctatcagactgccttcaacagccctgacgtgaagaaggcagcctaa

SEQ ID NO: 101 - RNA, 771 nt, synthetic construct
  source 1..771: mol_type = other RNA
atgaaggccaagctgctggtgctgctgtgcactttcaccgcaacatacgccgtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacccccaacgacgtggccaccgtgtatgtgagaaccagcttcagcaaaacaatctggcaggtcgccgccaacgccgacagatacatcgcaggccacaagagtgctgccgtggcgaaaaccctggcacacgcaattgccaaggccgcaggcgtgcgggccaatcctaagaccactgtgggcaatatccaggccgccaggactgaactgctggctgcctaccagacagccttcaacagcccagacgtgaagaaggccgcctaa

SEQ ID NO: 102 - RNA, 1029 nt, synthetic construct
  source 1..1029: mol_type = other RNA
atgaaggcaaagctgctggtgctgctgtgcacatttactgccacttacgcaagctccaaccggccaagaagcgtcgcccaggcagccatcgcaacagacggcaaggggatcatcaataaggactgcagagacgccgtgatcaatgatgccaaactgagggccgccatcgccggagctctggtgaaggccgggttttctagcgccgacgctgtggcactggcacctagaatcgctcgggaaatggccaaggagggggttctgctgattaaccaccacaagctgaaagcactgatcggagcccaggtgggactgctgacagatgccaagatccagagagccgcagctgcagtggacctgggcatcaaagccacactggccgcaaccattatccccaatgccctgcactccgcagccttcaaggatgctgtggtggccaacctggtggcagctggggttgataagaagctggccaaggccaccgcagttgccattgctgccacagccctgaatcctgcactgggacccatcgcaaagactgaggcaatcaaggccgagattggcgcacaggccgctctgctggtgagcagaggagtgcacctgaagaaggccgccattgagcacatcattgggagatcctttgacgccgcagtggccaccgccattgttagctcccccatcctgaatgcccggatcgtgacccacctcgtgcgggcaggaattgacaagagcctcgcagtgcagattgccccacggattatagatagactggccaaggagccgctgctggccctgaacactgcaaagctgatgaaaaacatcacaaggcagatcgtcgacgtgattaccgccgacaaggcaattaagaccgccgagcagctggagaaggagctgcctgccctggacgacctggtgaagaaggcctgctcctcccccaagcctactccaaccattctggcaatctatagcaccgtggctagcagcctggtgctgctggtgtctctgggagccatcagcttcggctctggctaa

SEQ ID NO: 103 - RNA, 930 nt, synthetic construct
  source 1..930: mol_type = other RNA
atgaaggccaagctgctggtgctgctgtgcacattcacagcaacctatgcatccagcaaccggcctagaagcgtggcccaggctgccatcgccacagatggcaagggcatcatcaacaaggactgtagagacgccgtgatcaatgacgccaagctgagagccgccatcgccggagccctggtgaaggccggctttagcagcgccgacgccgtggccctggcccctcggatcgcccgggagatggccaaggagggagtgctgctgatcaatcatcacaagctgaaggccctgattggcgcccaggtgggactgctgaccgacgccaagattcagagagccgccgctgccgtggatctgggcatcaaggccaccctggccgccaccatcatccctaacgccctgcactccgccgcttttaaggatgccgtggtggctaacctggtggctgccggcgtggacaagaagctggccaaagctaccgccgttgccatcgccgctacagctctgaatcctgccctggggcccatcgccaagacagaggccattaaggccgaaattggagcccaggccgcactgctggtgtccagaggcgtgcacctgaagaaggccgccatcgagcacatcattggcagatccttcgatgctgccgtggctaccgccatcgtgagcagccctatcctgaatgccagaatcgtgacccacctggtgagagcaggcatcgacaagagcctggccgtgcagatcgcccctcggattatcgaccggctggccaaggagcccctgctggccctgaacaccgccaagctgatgaagaacatcaccagacagatcgtggatgtgatcaccgccgataaggccatcaaaacagccgagcagctggagaaggagctgcctgccctggatgacctggtgaagaaggcctgtagctaa

SEQ ID NO: 104 - RNA, 1017 nt, synthetic construct
  source 1..1017: mol_type = other RNA
atgaaggcaaagctgctggtgctgctgtgcacatttactgccacttacgcaagctccaaccggccaagaagcgtcgcccaggcagccatcgcaacagacggcaaggggatcatcaataaggactgcagagacgccgtgatcaatgatgccaaactgagggccgccatcgccggagctctggtgaaggccgggttttctagcgccgacgctgtggcactggcacctagaatcgctcgggaaatggccaaggagggggttctgctgattaaccaccacaagctgaaagcactgatcggagcccaggtgggactgctgacagatgccaagatccagagagccgcagctgcagtggacctgggcatcaaagccacactggccgcaaccattatccccaatgccctgcactccgcagccttcaaggatgctgtggtggccaacctggtggcagctggggttgataagaagctggccaaggccaccgcagttgccattgctgccacagccctgaatcctgcactgggacccatcgcaaagactgaggcaatcaaggccgagattggcgcacaggccgctctgctggtgagcagaggagtgcacctgaagaaggccgccattgagcacatcattgggagatcctttgacgccgcagtggccaccgccattgttagctcccccatcctgaatgcccggatcgtgacccacctcgtgcgggcaggaattgacaagagcctcgcagtgcagattgccccacggattatagatagactggccaaggagccgctgctggccctgaacactgcaaagctgatgaaaaacatcacaaggcagatcgtcgacgtgattaccgccgacaaggcaattaagaccgccgagcagctggagaaggagctgcctgccctggacgacctggtgaagaaggcctgctccagcggcagcattctggcaatctatagcaccgtggctagcagcctggtgctgctggtgtctctgggagccatcagcttcggctctggctaa

SEQ ID NO: 105 - RNA, 1029 nt, synthetic construct
  source 1..1029: mol_type = other RNA
atgaaggcaaagctgctggtgctgctgtgcacatttactgccacttacgcagccagcaatggcaacagctccatcacccagagcgccgccttcagccctagagctaccacaaagatcagcgaagattgcagaaaggccattatcaatgatctgaaactgagaggcgccatcgtgggagctctggtgaaggccggcctgtctgccgccgacgccgccgccctggcccctagaattgccgccgaaatggccgccgaaggcaccctgaccatcaatcaccaccggctgaaggtgctggtcgcctctcaactgggcctggtggccgatgctgcagtgcaacatgccgcagccgccatcgacctgagcttcaaggccatcctgggcgccagcatcatccctaatgccctgggctccgccgctttcaagaatgccgtgatcgctaatctcgtggccgccggcattgacaagcacctggccagagccacagcagtggccatcgttgccacagcactgaatcccgctctgggtcctatcgccaagttcgagctgatcaaggccgagatcgccgcccaggccgccctgctgatcagaagaggcgtgcatctgcagaaagctgccatcgagcacgtgatcggacgggcattcgacgctgccgtggccacagccatcatcagcagccctatcctgtctgccagaatcgtgacacacctggtgagagccggcattgacaaatctatcgccatcagcctggcccctcacatcgtgaagagactggccaaggagcccctgctggccttcaatacagccaagctggtgaaggatatcgccagacagattgtggacatccggaatacccaggaggccatcgccgtgtacaagcagctgaaggccgagctgcctaccctggatggcctggtgcagaaggcttgcaccccagaacctacacccaccattctggcaatctatagcaccgtggctagcagcctggtgctgctggtgtctctgggagccatcagcttcggctctggctaa

SEQ ID NO: 106 - RNA, 1020 nt, synthetic construct
  source 1..1020: mol_type = other RNA
atgaaggcaaagctgctggtgctgctgtgcacatttactgccacttacgcagccagcaatggcaacagctccatcacccagagcgccgccttcagccctagagctaccacaaagatcagcgaagattgcagaaaggccattatcaatgatctgaaactgagaggcgccatcgtgggagctctggtgaaggccggcctgtctgccgccgacgccgccgccctggcccctagaattgccgccgaaatggccgccgaaggcaccctgaccatcaatcaccaccggctgaaggtgctggtcgcctctcaactgggcctggtggccgatgctgcagtgcaacatgccgcagccgccatcgacctgagcttcaaggccatcctgggcgccagcatcatccctaatgccctgggctccgccgctttcaagaatgccgtgatcgctaatctcgtggccgccggcattgacaagcacctggccagagccacagcagtggccatcgttgccacagcactgaatcccgctctgggtcctatcgccaagttcgagctgatcaaggccgagatcgccgcccaggccgccctgctgatcagaagaggcgtgcatctgcagaaagctgccatcgagcacgtgatcggacgggcattcgacgctgccgtggccacagccatcatcagcagccctatcctgtctgccagaatcgtgacacacctggtgagagccggcattgacaaatctatcgccatcagcctggcccctcacatcgtgaagagactggccaaggagcccctgctggccttcaatacagccaagctggtgaaggatatcgccagacagattgtggacatccggaatacccaggaggccatcgccgtgtacaagcagctgaaggccgagctgcctaccctggatggcctggtgcagaaggcttgcaccagcggcagcattctggcaatctatagcaccgtggctagcagcctggtgctgctggtgtctctgggagccatcagcttcggctctggctaa

SEQ ID NO: 107 - RNA, 933 nt, synthetic construct
  source 1..933: mol_type = other RNA
atgaaggccaagctgctggtgctgctgtgcacattcacagcaacctatgcagctagcaatggaaactcctctattacacagtctgccgccttcagcccaagagctaccacaaagatcagcgaggattgtagaaaggccatcattaacgatctgaagctgcggggcgccatcgtgggcgctctcgtgaaagccggactgtctgcagcagacgctgcagcactggctccaaggatcgccgctgagatggcagcagaggggacactgactattaaccaccataggctgaaagtgctggtcgcttcccagctgggcctggttgcagatgcagccgtgcagcatgctgccgctgcaattgacctgagcttcaaagctatcctgggcgcctctatcattccaaatgctctcgggagcgcagccttcaaaaacgccgtgattgccaacctggtggctgcaggaattgacaagcacctggccagagccactgctgtggcaattgtggccactgctctgaatccagcactgggacccatcgcaaagtttgagctgattaaggcagaaattgccgctcaagcagccctgctgattagacggggagtgcacctgcagaaggccgcaattgaacacgtgatcggccgggcatttgacgccgctgttgctactgccatcatcagcagccctattctgtccgccaggattgtcacccacctggtcagggcaggcattgataagagcattgctatcagcctggccccacacatcgttaagcggctggccaaagaacccctgctggcttttaatactgctaagctggtgaaagacattgcccgccagatcgtggacattaggaatactcaggaggccatcgccgtgtataaacagctcaaggcagaactgcctaccctggacggcctggtgcagaaagcctgtacataa

SEQ ID NO: 108 - RNA, 933 nt, synthetic construct
  source 1..933: mol_type = other RNA
atgaaggccaagctgctggtgctgctgtgcacattcacagcaacctatgcagctagcaatggaaactcctctattacacagtctgccgccttcagcccaagagctaccacaaagatcagcgaggattgtagaaaggccatcattaacgatctgaagctgcggggcgccatcgtgggcgctctcgtgaaagccggactgtctgtggcagacgctgcagcactggctccaaggatcgccgctgagatggcagcagaggggacactgactattaaccaccataggctgaaagtgctggtcgcttcccagctgggcctggttgcagatgcagccgtgcagcatgctgccgctgcaattgacctgagcttcaaagctatcctgggcgcctctatcattccaaatgctctcgggagcgcagccttcaaaaacgccgtgattgccaacctggtggctgcaggaattgacaagcacctggccagagccactgctgtggcaattgtggccactgctctgaatccagcactgggacccatcgcaaagtttgagctgattaaggcagaaattgccgctcaagcagccctgctgattagacggggagtgcacctgcagaaggccgcaattgaacacgtgatcggccgggcatttgacgccgctgttgctactgccatcatcagcagccctattctgtccgccaggattgtcacccacctggtcagggcaggcattgataagagcattgctatcagcctggccccacacatcgttaagcggctggccaaagaacccctgctggcttttaatactgctaagctggtgaaagacattgcccgccagatcgtggacattaggaatactcaggaggccatcgccgtgtataaacagctcaaggcagaactgcctaccctggacggcctggtgcagaaagcctgtacataa

SEQ ID NO: 109 - RNA, 1029 nt, synthetic construct
  source 1..1029: mol_type = other RNA
atgaaggcaaagctgctggtgctgctgtgcacatttactgccacttacgcagccagcaatggcaacagctccatcacccagagcgccgccttcagccctagagctaccacaaagatcagcgaagattgcagaaaggccattatcaatgatctgaaactgagaggcgccatcgtgggagctctggtgaaggccggcctgtctgtggccgacgccgccgccctggcccctagaattgccgccgaaatggccgccgaaggcaccctgaccatcaatcaccaccggctgaaggtgctggtcgcctctcaactgggcctggtggccgatgctgcagtgcaacatgccgcagccgccatcgacctgagcttcaaggccatcctgggcgccagcatcatccctaatgccctgggctccgccgctttcaagaatgccgtgatcgctaatctcgtggccgccggcattgacaagcacctggccagagccacagcagtggccatcgttgccacagcactgaatcccgctctgggtcctatcgccaagttcgagctgatcaaggccgagatcgccgcccaggccgccctgctgatcagaagaggcgtgcatctgcagaaagctgccatcgagcacgtgatcggacgggcattcgacgctgccgtggccacagccatcatcagcagccctatcctgtctgccagaatcgtgacacacctggtgagagccggcattgacaaatctatcgccatcagcctggcccctcacatcgtgaagagactggccaaggagcccctgctggccttcaatacagccaagctggtgaaggatatcgccagacagattgtggacatccggaatacccaggaggccatcgccgtgtacaagcagctgaaggccgagctgcctaccctggatggcctggtgcagaaggcttgcaccccagaacctacacccaccattctggcaatctatagcaccgtggctagcagcctggtgctgctggtgtctctgggagccatcagcttcggctctggctaa

SEQ ID NO: 110 - RNA, 1020 nt, synthetic construct
  source 1..1020: mol_type = other RNA
atgaaggccaagctgctggtgctgctgtgcacattcacagcaacctatgcagctagcaatggaaactcctctattacacagtctgccgccttcagcccaagagctaccacaaagatcagcgaggattgtagaaaggccatcattaacgatctgaagctgcggggcgccatcgtgggcgctctcgtgaaagccggactgtctgtggcagacgctgcagcactggctccaaggatcgccgctgagatggcagcagaggggacactgactattaaccaccataggctgaaagtgctggtcgcttcccagctgggcctggttgcagatgcagccgtgcagcatgctgccgctgcaattgacctgagcttcaaagctatcctgggcgcctctatcattccaaatgctctcgggagcgcagccttcaaaaacgccgtgattgccaacctggtggctgcaggaattgacaagcacctggccagagccactgctgtggcaattgtggccactgctctgaatccagcactgggacccatcgcaaagtttgagctgattaaggcagaaattgccgctcaagcagccctgctgattagacggggagtgcacctgcagaaggccgcaattgaacacgtgatcggccgggcatttgacgccgctgttgctactgccatcatcagcagccctattctgtccgccaggattgtcacccacctggtcagggcaggcattgataagagcattgctatcagcctggccccacacatcgttaagcggctggccaaagaacccctgctggcttttaatactgctaagctggtgaaagacattgcccgccagatcgtggacattaggaatactcaggaggccatcgccgtgtataaacagctcaaggcagaactgcctaccctggacggcctggtgcagaaagcctgtacaagcggctctattctggccatctacagcacagtggccagctctctggtgctgctggttagcctgggcgccatcagcttcggcagcggctaa

SEQ ID NO: 111 - RNA, 1020 nt, synthetic construct
  source 1..1020: mol_type = other RNA
atgaaggccaagctgctggtgctgctgtgcacattcacagcaacctatgcagctagcaatggacagtcctctattacacagtctgccgccttcagcccaagagctaccacaaagatcagcgaggattgtagaaaggccatcattaacgatctgaagctgcggggcgccatcgtgggcgctctcgtgaaagccggactgtctgcagcagacgctgcagcactggctccaaggatcgccgctgagatggcagcagaggggacactgactattaaccaccataggctgaaagtgctggtcgcttcccagctgggcctggttgcagatgcagccgtgcagcatgctgccgctgcaattgacctgagcttcaaagctatcctgggcgcctctatcattccaaatgctctcgggagcgcagccttcaaaaacgccgtgattgccaacctggtggctgcaggaattgacaagcacctggccagagccactgctgtggcaattgtggccactgctctgaatccagcactgggacccatcgcaaagtttgagctgattaaggcagaaattgccgctcaagcagccctgctgattagacggggagtgcacctgcagaaggccgcaattgaacacgtgatcggccgggcatttgacgccgctgttgctactgccatcatcagcagccctattctgtccgccaggattgtcacccacctggtcagggcaggcattgataagagcattgctatcagcctggccccacacatcgttaagcggctggccaaagaacccctgctggcttttaatactgctaagctggtgaaagacattgcccgccagatcgtggacattaggaatactcaggaggccatcgccgtgtataaacagctcaaggcagaactgcctaccctggacggcctggtgcagaaagcctgtacaagcggctctattctggccatctacagcacagtggccagctctctggtgctgctggttagcctgggcgccatcagcttcggcagcggctaa

SEQ ID NO: 112 - RNA, 1029 nt, synthetic construct
  source 1..1029: mol_type = other RNA
atgaaggccaagctgctggtgctgctgtgcacattcacagcaacctatgcagctagcaatggacagtcctctattacacagtctgccgccttcagcccaagagctaccacaaagatcagcgaggattgtagaaaggccatcattaacgatctgaagctgcggggcgccatcgtgggcgctctcgtgaaagccggactgtctgcagcagacgctgcagcactggctccaaggatcgccgctgagatggcagcagaggggacactgactattaaccaccataggctgaaagtgctggtcgcttcccagctgggcctggttgcagatgcagccgtgcagcatgctgccgctgcaattgacctgagcttcaaagctatcctgggcgcctctatcattccaaatgctctcgggagcgcagccttcaaaaacgccgtgattgccaacctggtggctgcaggaattgacaagcacctggccagagccactgctgtggcaattgtggccactgctctgaatccagcactgggacccatcgcaaagtttgagctgattaaggcagaaattgccgctcaagcagccctgctgattagacggggagtgcacctgcagaaggccgcaattgaacacgtgatcggccgggcatttgacgccgctgttgctactgccatcatcagcagccctattctgtccgccaggattgtcacccacctggtcagggcaggcattgataagagcattgctatcagcctggccccacacatcgttaagcggctggccaaagaacccctgctggcttttaatactgctaagctggtgaaagacattgcccgccagatcgtggacattaggaatactcaggaggccatcgccgtgtataaacagctcaaggcagaactgcctaccctggacggcctggtgcagaaagcctgtacacccgagcctacacctaccattctggccatctacagcacagtggccagctctctggtgctgctggttagcctgggcgccatcagcttcggcagcggctaa

SEQ ID NO: 113 - RNA, 969 nt, synthetic construct
  source 1..969: mol_type = other RNA
atgaaggccaagctgctggtgctgctgtgcacattcacagcaacctatgcatcatccaacaggcctcgctccgtggcccaggctgctattgctaccgacggcaagggaatcatcgataaagactccagggatgccgtgatcaacgatgccaagctccgggccgcaattgccggagccctggtgaaagccgggttcagctccgccgatgctgtggctctggctccaaggattgctaaggagatggccaaagagggggtgctgctgattaaccaccacaagctgaaggcactgatcggagcacagctgggactgctgactgacgccaaaatccagcgcgccgctgcagcagtcgacctgggcatcaaggccacactggctgctaccatcattcctaacgctctgggcagcgcagcctttaagaatgccgttatcgccaatctcgtggctgctggaattgataagcacctggccagagccacagctgtggccatcgtggccactgccctgaacccagcattgggccctatcgctaagtttgagctgattaaggccgagatcgccgctcaggctgccctgctgattcgcaggggagtgcacctgcagaaggctgccatcgagcacgtcatcggcagagcattcgacgccgctgtcgccactgctatcatctctagcccaatcctgagcgccaggatcgtcactcacctggtgagagctgggattgacaaatccatcgctatcagcctggcccctcacatcgtgaagaggctggctaaggaaccactgctggcttttaacactgctaagctgatgaagaatattacaagacagatcgtggacgtcatcaccgccgacaaagccatcaagacagccgagcagctcgagaaggagctgcctgccctggatgacctggtgaagaaggcatcctccccccctaagccaacccctaccccaacccctacacctacttaa

SEQ ID NO: 114 - RNA, 969 nt, synthetic construct
  source 1..969: mol_type = other RNA
atgaaggccaagctgctggtgctgctgtgcacattcacagcaacctatgcatcatccaacaggcctcgctccgtggcccaggctgctattgctaccgacggcaagggaatcatcgataaagactccagggatgccgtgatcaacgatgccaagctccgggccgcaattgccggagccctggtgaaagccgggttcagctccgccgatgctgtggctctggctccaaggattgctaaggagatggccaaagagggggtgctgctgattaaccaccacaagctgaaggcactgatcggagcacagctgggactgctgactgacgccaaaatccagcgcgccgctgcagcagtcgacctgggcatcaaggccacactggctgctaccatcattcctaacgctctgggcagcgcagcctttaagaatgccgttatcgccaatctcgtggctgctggaattgataagcacctggccagagccacagctgtggccatcgtggccactgccctgaacccagcattgggccctatcgctaagtttgagctgattaaggccgagatcgccgctcaggctgccctgctgattcgcaggggagtgcacctgcagaaggctgccatcgagcacgtcatcggcagagcattcgacgccgctgtcgccactgctatcatctctagcccaatcctgagcgccaggatcgtcactcacctggtgagagctgggattgacaaatccatcgctatcagcctggcccctcacatcgtgaagaggctggctaaggaaccactgctggcttttaacactgctaagctgatgaagcagattacaagacagatcgtggacgtcatcaccgccgacaaagccatcaagacagccgagcagctcgagaaggagctgcctgccctggatgacctggtgaagaaggcatcctccccccctaagccaacccctaccccaacccctacacctacttaa

SEQ ID NO: 115 - RNA, 1251 nt, synthetic construct
  source 1..1251: mol_type = other RNA
atgaaggcaaagctgctggtgctgctgtgcacatttactgccacttacgcagcaggccctaccgtcacagtgatccctgtgggcagagaaggtggcgacatcaccatcagcggcaagggcttcagcaccacaggctttggcgtgtacgtggccgtggcacctgctagcgtgcccgaattctacggcaatagcgacaagttctacggctacgaccccagcaaggacaccaccgagagccctagcaccatctgggtgtacacccctagccagaaggccatcggctctcggttcgcccagggcagacccatgaacaatgacggcagcttcacaatcaccatgaaggccccccctttcgagcagggcaaagacttcgtggtgctgaccacaaaggcccatggcgtgggcaaaaccgatcacagcgacgacacacggacacctgtcacctacagagaggctacccctgcccctacaggccctaagacacccatcgcccctagcaagcagcctagcaagcaggccgcccctagcaagcaggtgaagccttctaagcaggccggccccaacaagcagtccaccacacctcagcagaagacagccgagcacagaagccagacccctgccgcccaccggaccatgacaaagcaggtgagcaccatcggcgccagcaaggtgacaagcggctctctgacctggggcatcagaacctcttttacctcctacctgagaggccctatcgccaacggctcttggaagctgagcggaggcgccaattggaatggctccgccttcaccttccctctgaccagcggcagcttcgacccagccaccaagtccggaagcctgaagtacagcggcagcgtgcacatgacaggccaccacggcatcctggacatgaccctggccgagccaagcctgcagatcaagggaagcaccggccacctgtatctggacgtgaaaagcagcagcatggacggcaagaagaccaattacggcagagtggacttcgccaccttcggagtgagcgtgtctggcaacgccgccatcaagggcagccctgtgaagctgaccgccacaggcgctaaggcttttgccggcttctacagagccggcgagcctatgaaccctctgagcaccaatctgaccctgagcgccgagaaggtgtcccacaacgtgaccgtggacgcagtgacaggcaaggtgatcggcgacgactctggcaagggagccggccggggcctgcccgtgacataa

SEQ ID NO: 116 - skipped

SEQ ID NO: 117 - RNA, 1251 nt, synthetic construct
  source 1..1251: mol_type = other RNA
atgaaggccaagctgctggtgctgctgtgcacattcacagcaacctatgcagctgggccaacagtgacagtgatccccgtgggacgcgagggcggagacattaccatctccgggaagggctttagcacaacagggttcggtgtgtatgtggctgtggccccagcaagcgtgccagagttctacgggaactccgataaattttatgggtatgaccccagtaaagacacaaccgagagccccagcacaatttgggtttacaccccctctcagaaagctattggctcccggttcgcccagggcagaccaatgaataatgacgggagctttaccatcactatgaaggcccccccattcgagcagggaaaggactttgtggtcctgactaccaaggctcacggagtgggcaaaaccgaccactctgacgacactcggacaccggtgacataccgggaggctactcccgctccaacaggccctaaaactccaatcgcccccagcaagcagccttcaaagcaggctgcccccagcaagcaggtgaagcctagcaagcaggctggccccaacaagcagagcactactcctcagcagaagaccgcagagcacagatctcagaccccagcagctcatcgcactatgactaagcaggtgtgtaccatcggagcctcaaaggtgaccagcggatccctgacatggggaatccggactagctttacttcctacctgcgcgggcccatcgcaaacggcagctggaagctgtctgggggggccaattggaacggcagcgccttcacattcccactgacttctggatccttcgaccccgccaccaaatccggctcactgaagtacagcgggagcgtgcacatgactggacaccatggaattctggacatgacactcgctgaaccctctctgcagatcaagggctctactgggcacctgtatctggatgtgaagtcctcttctatggacggcaagaagacaaactatgggagagtggattttgccaccttcggagtgagcgtgagcggaaatgctgcaatcaaagggtcccccgtgaaactgaccgctaccggagcaaaagcctttgccggcttttacagagcaggagagcccatgaaccccctgagcaccaatctgacactgagcgctgagaaagtttgtcacaatgtgaccgttgatgcagttaccgggaaagtgatcggcgacgattccggaaagggggcaggaaggggactgcctgtcacttaa

SEQ ID NO: 118 - RNA, 1251 nt, synthetic construct
  source 1..1251: mol_type = other RNA
atgaaggccaagctgctggtgctgctgtgcacattcacagcaacctatgcagctgggccaacagtgacagtgatccccgtgggacgcgagggcggagacattaccatctccgggaagggctttagcacaacagggttcggtgtgtatgtggctgtggccccagcaagcgtgccagagttctacgggaactccgataaattttatgggtatgaccccagtaaagacacaaccgagagccccagcacaatttgggtttacaccccctctcagaaagctattggctcccggttcgcccagggcagaccaatgaataatgacgggagctttaccatcactatgaaggcccccccattcgagcagggaaaggactttgtggtcctgactaccaaggctcacggagtgggcaaaaccgaccactctgacgacactcggacaccggtgacataccgggaggctactcccgctccaacaggccctaaaactccaatcgcccccagcaagcagccttcaaagcaggctgcccccagcaagcaggtgaagcctagcaagcaggctggccccaacaagcagagcactactcctcagcagaagaccgcagagcacagatctcagaccccagcagctcatcgcactatgactaagcaggtgtgtaccatcggagcctcaaaggtgaccagcggatccctgacatggggaatccggactagctttacttcctacctgcgcgggcccatcgcaaagggcagctggaagctgtctgggggggccaattggaacggaggcgccttcacattcccactgacttctggatccttcgaccccgccaccaaatccggctcactgaagtacagcgggagcgtgcacatgactggacaccatggaattctggacatgacactcgctgaaccctctctgcagatcaagggctctactgggcacctgtatctggatgtgaagtcctcttctatggacggcaagaagacaaactatgggagagtggattttgccaccttcggagtgagcgtgagcggaaatgctgcaatcaaagggtcccccgtgaaactgaccgctaccggagcaaaagcctttgccggcttttacagagcaggagagcccatgaaccccctgagcacccagctgacactgagcgctgagaaagtttgtcacagcgtgaccgttgatgcagttaccgggaaagtgatcggcgacgattccggaaagggggcaggaaggggactgcctgtcacttaa

SEQ ID NO: 119 - RNA, 1251 nt, synthetic construct
  source 1..1251: mol_type = other RNA
atgaaggcaaagctgctggtgctgctgtgcacatttactgccacttacgcagcaggccctaccgtcacagtgatccctgtgggcagagaaggtggcgacatcaccatcagcggcaagggcttcagcaccacaggctttggcgtgtacgtggccgtggcacctgctagcgtgcccgaattctacggcaatagcgacaagttctacggctacgaccccagcaaggacaccaccgagagccctagcaccatctgggtgtacacccctagccagaaggccatcggctctcggttcgcccagggcagacccatgaacaatgacggcagcttcacaatcaccatgaaggccccccctttcgagcagggcaaagacttcgtggtgctgaccacaaaggcccatggcgtgggcaaaaccgatcacagcgacgacacacggacacctgtcacctacagagaggctacccctgcccctacaggccctaagacacccatcgcccctagcaagcagcctagcaagcaggccgcccctagcaagcaggtgaagccttctaagcaggccggccccaacaagcagtccaccacacctcagcagaagacagccgagcacagaagccagacccctgccgcccaccggaccatgacaaagcaggtgagcaccatcggcgccagcaaggtgacaagcggctctctgacctggggcatcagaacctcttttacctcctacctgagaggccctatcgccaagggctcttggaagctgagcggaggcgccaattggaatggcggcgccttcaccttccctctgaccagcggcagcttcgacccagccaccaagtccggaagcctgaagtacagcggcagcgtgcacatgacaggccaccacggcatcctggacatgaccctggccgagccaagcctgcagatcaagggaagcaccggccacctgtatctggacgtgaaaagcagcagcatggacggcaagaagaccaattacggcagagtggacttcgccaccttcggagtgagcgtgtctggcaacgccgccatcaagggcagccctgtgaagctgaccgccacaggcgctaaggcttttgccggcttctacagagccggcgagcctatgaaccctctgagcacccagctgaccctgagcgccgagaaggtgtcccacagcgtgaccgtggacgcagtgacaggcaaggtgatcggcgacgactctggcaagggagccggccggggcctgcccgtgacataa

SEQ ID NO: 120 - RNA, 1338 nt, synthetic construct
  source 1..1338: mol_type = other RNA
atgaaggcaaagctgctggtgctgctgtgcacatttactgccacttacgcagccggtccaacagtgacagtgatccctgtgggcagagaaggcggcgatatcacaatcagcggcaagggcttctctacaacaggattcggagtgtatgtggctgtcgcccctgccagcgtgcccgaattctatggaaacagcgacaagttctacggctacgaccccagcaaggatacaaccgagagcccctccaccatctgggtgtacacccccagccagaaggccatcggcagcagattcgcccagggcagacctatgaataatgacggctccttcaccatcaccatgaaggcccctccattcgagcagggcaaggatttcgtggtgctgaccaccaaagcccacggcgtgggcaaaacagaccacagcgatgatacccggacccccgtgacatacagagaagccacaccagcccctaccggccctaagacccctatcgccccttctaagcagcctagcaagcaggccgccccatccaaacaggtgaagcctagcaagcaggccgggcccaataagcagagcacaaccccccagcagaagaccgccgaacacagaagccagacacccgccgcccacagaacaatgaccaagcaggtgtgcaccatcggcgcctccaaggtgaccagcggcagcctgacctggggcattagaaccagctttaccagctacctgagaggccctatcgccaacggcagctggaagctgtccggcggcgccaattggaacggcagcgcctttaccttccccctgacaagcggcagcttcgaccctgccaccaagtctggcagcctgaagtacagcggcagcgtgcatatgaccggccaccacggcatcctggatatgaccctggccgaacctagcctgcagatcaagggcagcaccggccacctgtacctggacgtgaaaagcagcagcatggacggcaagaagacaaattacggcagagtggacttcgccaccttcggcgtgagcgtgagcggaaatgccgccatcaaaggaagccctgtgaagctgaccgccaccggcgccaaggcctttgccggcttctacagagccggcgagcctatgaatcctctgagcaccaatctgaccctgagcgccgaaaaggtgtgccacaatgtgacagtcgacgccgtgacaggcaaggtcatcggcgatgactctggcaaaggagccggcagaggcctgcccgtgacaagcggcagcattctggcaatctatagcaccgtggctagcagcctggtgctgctggtgtctctgggagccatcagcttcggctctggctaa

SEQ ID NO: 121 - RNA, 1338 nt, synthetic construct
  source 1..1338: mol_type = other RNA
atgaaggccaagctgctggtgctgctgtgcacattcacagcaacctatgcagctgggccaacagtgacagtgatccccgtgggacgcgagggcggagacattaccatctccgggaagggctttagcacaacagggttcggtgtgtatgtggctgtggccccagcaagcgtgccagagttctacgggaactccgataaattttatgggtatgaccccagtaaagacacaaccgagagccccagcacaatttgggtttacaccccctctcagaaagctattggctcccggttcgcccagggcagaccaatgaataatgacgggagctttaccatcactatgaaggcccccccattcgagcagggaaaggactttgtggtcctgactaccaaggctcacggagtgggcaaaaccgaccactctgacgacactcggacaccggtgacataccgggaggctactcccgctccaacaggccctaaaactccaatcgcccccagcaagcagccttcaaagcaggctgcccccagcaagcaggtgaagcctagcaagcaggctggccccaacaagcagagcactactcctcagcagaagaccgcagagcacagatctcagaccccagcagctcatcgcactatgactaagcaggtgtgtaccatcggagcctcaaaggtgaccagcggatccctgacatggggaatccggactagctttacttcctacctgcgcgggcccatcgcaaagggcagctggaagctgtctgggggggccaattggaacggaggcgccttcacattcccactgacttctggatccttcgaccccgccaccaaatccggctcactgaagtacagcgggagcgtgcacatgactggacaccatggaattctggacatgacactcgctgaaccctctctgcagatcaagggctctactgggcacctgtatctggatgtgaagtcctcttctatggacggcaagaagacaaactatgggagagtggattttgccaccttcggagtgagcgtgagcggaaatgctgcaatcaaagggtcccccgtgaaactgaccgctaccggagcaaaagcctttgccggcttttacagagcaggagagcccatgaaccccctgagcacccagctgacactgagcgctgagaaagtttgtcacagcgtgaccgttgatgcagttaccgggaaagtgatcggcgacgattccggaaagggggcaggaaggggactgcctgtcactagcggctctattctggccatctacagcacagtggccagctctctggtgctgctggttagcctgggcgccatcagcttcggcagcggctaa

SEQ ID NO: 122 - RNA, 1410 nt, synthetic construct
  source 1..1410: mol_type = other RNA
atgaaggccaaactgctggtgctgctgtgtacctttacagccacatatgcctcctctaatagaccaagatccgtggctcaggccgccatcgctacagatgggaaaggaatcatcgataaggatagtcgggatgcagtgatcaacgacgccaaactccgagctgccatcgccggcgccctggtgaaggctgggttttccagcgccgacgcagtggccctggcacctagaatcgccaaggagatggcaaaggagggcgtcctgctgattaatcaccacaagctgaaagctctgattggggctcagctcggactgctgactgacgccaagatccagagggccgcagctgccgtggatctggggatcaaggccactctggccgctactattattcctaacgctctggggtctgccgccttcaagaatgctgtcattgccaacctggtggctgctggcatcgacaaacacctcgccagggctaccgctgtggccatcgttgccacagccctgaatcctgccctgggcccaatcgctaaatttgagctgattaaggctgagatcgccgcccaggccgcactgctgatcagaaggggggtccatctgcagaaggccgctattgaacatgtgatcggccgcgcattcgacgctgccgtggccaccgctatcatttcctctccaatcctgagcgcacggattgtgacccacctggtgagggctggaattgataaaagcatcgctatctccctggctcctcatatcgtgaagcgcctggccaaggagcccctgctggctttcaacaccgccaagctgatgaagaatattacaagacagatcgttgacgtgatcacagccgataaagccatcaaaacagctgagcagctggaaaaggagctccccgccctggacgacctggtcaaaaaagctagctcccctcctaagcctacccccacacctactccaacccccaccgccgggcccaccgtgacagtgatccccgtgggaagggaggggggcgacattaccattagcggaaaagggttctccacaaccgggttcggcgtctacgtggccgtggctcctgcctctgtgccagagttctatggcaatagcgacaagttctatgggtacgatccatctaaggacaccaccgaaagtcccagcaccatctgggtgtatacaccatcccagaaagcaatcggcagcagattcgcccaaggcaggcccatgaataatgatggctctttcaccattacaatgaaggcccctcctttcgagcagggaaaggatttcgtggtgctgaccaccaaagcacatggcgttggaaagaccgaccacagcgacgatactcggacccctgtgacttacagagaggccacccctgctccaaccggccctaaaactcccatctgataa

SEQ ID NO: 123 - RNA, 927 nt, synthetic construct
  source 1..927: mol_type = other RNA
atgaaggccaagctcctggtgctgctgtgtaccttcactgccacttacgccgtggctcaggccgctattgctacagatggaaagggcatcatcgataaggactcccgcgatgccgtgatcaatgatgccaagctgagggctgctattgctggcgccctggtgaaggctggcttcagctccgccgatgcagtggcactggctcctcggatcgctaaggagatggccaaggagggcgtgctcctgatcaaccaccacaaactgaaggccctgattggggcccagctgggcctgctcacagacgccaagatccagcgggccgctgcagccgtggacctgggcatcaaggccactctggctgctacaatcatcccaaacgcactcgggtccgccgccttcaagaatgccgtgatcgccaacctggttgcagccggaatcgacaaacatctggccagagccacagccgtggccatcgtggctaccgctctgaacccagctctggggcctatcgccaagtttgaactgatcaaggctgagatcgccgcacaggccgccctgctgatcagaagaggagtgcacctgcagaaagccgctatcgagcacgtgatcggacgcgcctttgacgcagccgtggctactgccatcatcagctctccaattctgagcgcaaggatcgtgacacacctggtgagggccggcatcgacaagagcatcgcaatttccctggcccctcacatcgtgaagcggctggctaaggagccactgctcgcctttaataccgcaaaactgatgaaggctattacacgccagatcgtggatgtgatcaccgctgataaggcaatcaagaccgccgaacagctggaaaaggaactgcctgccctggatgacctggtgaaaaaggctatgggcccccctaagcctacctgataa

SEQ ID NO: 124 - RNA, 1362 nt, synthetic construct
  source 1..1362: mol_type = other RNA
atgaaggccaagctgctggtgctgctgtgcaccttcaccgccacctacgcctccagcaaccggcctagaagcgtggcccaggccgccatcgccaccgacggcaagggcatcatcgacaaggattcccgggacgccgtgatcaatgacgccaagctgagagccgccatcgccggcgccctggtgaaggccggcttcagcagcgccgacgccgtggccctggcccccagaatcgccaaggagatggccaaggaaggcgtgctgctgatcaatcaccacaagctgaaggccctgatcggcgcccagctgggcctgctgacagatgccaagatccagagagccgctgccgccgtggacctgggcatcaaggccaccctggccgccaccatcatccctaacgccctgggcagcgctgccttcaagaatgccgtgatcgccaacctggtggccgccggcatcgacaagcacctggccagagccacagccgtggccatcgtggccaccgccctgaatcctgccctgggccccatcgccaagttcgagctgatcaaggccgagatcgccgcccaggccgccctgctgatccggcggggcgtgcacctgcagaaggccgccatcgaacacgtgatcggcagagccttcgatgccgccgtggccaccgccatcatcagctcccccatcctgagcgccagaatcgtgacccacctggtgcgggccggcatcgacaagtccatcgccattagcctggcccctcacatcgtgaagcgcctggccaaggagcccctgctggccttcaataccgccaagctgatgaagaatatcaccagacagatcgtggatgtgatcaccgccgataaggccatcaagaccgccgagcagctggagaaagagctgcccgccctggacgacctggtgaaaaaggccatgggcccccctaagcctacaggcggcggcggaggcgctggacctaccgtgaccgtgatccccgtgggcagagagggcggcgatatcaccatcagcggcaagggcttcagcaccaccggcttcggcgtgtacgtggccgtggcccctgccagcgtgcccgagttctacggcaatagcgacaagttctacggctacgaccccagcaaggacaccacagaatctcccagcaccatctgggtgtacacccccagccagaaggccattggcagccggttcgcccagggccggcctatgaacaatgatggcagcttcacaatcaccatgaaggccccacccttcgagcagggcaaggacttcgtggtgctgaccaccaaggcccacggagtgggcaagggcgaccacggcgacgacacacggacccccgtgggctacagatgataa

SEQ ID NO: 125 - RNA, 1371 nt, synthetic construct
  source 1..1371: mol_type = other RNA
atgaaagccaagctgctggtgctgctgtgtacttttactgcaacctacgccagcagcaaccgcccaaggtctgtcgcacaggctgctatcgccacagatggcaagggaatcattgataaggacagcagggatgctgtgatcaacgacgcaaaactgagagccgccatcgccggcgcactggtgaaagccggcttttcttccgccgacgcagtggctctggctcccagaatcgctaaggaaatggccaaggagggagtgctgctgatcaatcaccacaaactgaaggctctgatcggggctcagctgggcctgctgactgacgctaagattcagagagcagcagcagctgtcgacctgggaatcaaggcaaccctggcagccactatcattccaaatgctctgggctctgccgccttcaaaaacgccgtgattgccaatctcgtggcagctggaatcgataagcacctggcacgggccaccgccgtggctatcgtggccaccgctctgaatcccgccctgggccctattgccaagtttgaactgatcaaagccgagatcgctgcacaggccgccctgctcatccgcaggggggtccacctgcagaaagcagccatcgagcacgtgatcggcagggcctttgatgcagccgtcgccactgccatcatcagctctccaatcctgtctgcccggattgtgactcacctggtgcgcgccggcatcgataagtccatcgcaatctctctggccccacacatcgtgaagagactggccaaagaaccactcctggctttcaataccgccaaactcatgaaaaacatcacccggcagatcgtcgatgtgattaccgccgacaaggctatcaagaccgccgagcagctcgaaaaggagctgcctgccctggacgacctggtgaagaaagcctcctctccacccaaacccaccccaactccaactccaacccctacagctggccccaccgtgaccgtcatcccagtgggaagggaaggaggagacatcacaatctctggaaaggggtttagcacaaccggatttggagtgtacgtcgccgtggcccccgcatccgtgccagaattctacggcaattccgataaattctacgggtacgacccctccaaggacactacagagagcccctctacaatttgggtgtacaccccaagccagaaggccatcggaagcagatttgctcagggccggccaatgaacaacgacggatccttcacaatcacaatgaaagcacctccctttgagcagggcaaggattttgtggtgctgaccaccaaggctcacggggtcggaaagaccgatcacagcgacgatacaagaacccccgtgacataccggtgataa

SEQ ID NO: 126 - RNA, 51 nt, synthetic construct
  source 1..51: mol_type = other RNA
atgaaggccaagctgctggtgctgctgtgcacctttacagccacctacgcc

SEQ ID NO: 127 - RNA, 51 nt, synthetic construct
  source 1..51: mol_type = other RNA
atgaaagcaaagctgctggtgctgctgtgcaccttcaccgctacctacgcc

SEQ ID NO: 128 - RNA, 51 nt, synthetic construct
  source 1..51: mol_type = other RNA
atgaaggccaagctgctggtgctgctctgcaccttcaccgccacctacgcc

SEQ ID NO: 129 - RNA, 51 nt, synthetic construct
  source 1..51: mol_type = other RNA
atgaaggccaagctgctggtgctgctgtgcaccttcaccgccacctatgcc

SEQ ID NO: 130 - RNA, 51 nt, synthetic construct
  source 1..51: mol_type = other RNA
atgaaagccaagctgctggtgctgctctgcacttttaccgccacctacgcc

SEQ ID NO: 131 - RNA, 51 nt, synthetic construct
  source 1..51: mol_type = other RNA
atgaaggccaagctgctggtgctgctgtgcaccttcaccgccacatacgcc

SEQ ID NO: 132 - RNA, 51 nt, synthetic construct
  source 1..51: mol_type = other RNA
atgaaggccaagctgctggtgctgctgtgcaccttcaccgccacctacgcc

SEQ ID NO: 133 - RNA, 51 nt, synthetic construct
  source 1..51: mol_type = other RNA
atgaaggcaaagctgctggtgctgctgtgcaccttcaccgccacatacgca

SEQ ID NO: 134 - RNA, 51 nt, synthetic construct
  source 1..51: mol_type = other RNA
atgaaggccaagctgctggtgctgctgtgcaccttcacagccacctacgcc

SEQ ID NO: 135 - RNA, 51 nt, synthetic construct
  source 1..51: mol_type = other RNA
atgaaggccaagctgctggtgctgctgtgcactttcaccgcaacatacgcc

SEQ ID NO: 136 - RNA, 51 nt, synthetic construct
  source 1..51: mol_type = other RNA
atgaaggcaaagctgctggtgctgctgtgcacatttactgccacttacgca

SEQ ID NO: 137 - RNA, 51 nt, synthetic construct
  source 1..51: mol_type = other RNA
atgaaggccaagctgctggtgctgctgtgcacattcacagcaacctatgca

SEQ ID NO: 138 - RNA, 51 nt, synthetic construct; identical to SEQ ID NO: 136
  source 1..51: mol_type = other RNA

SEQ ID NO: 139 - RNA, 51 nt, synthetic construct; identical to SEQ ID NO: 136
  source 1..51: mol_type = other RNA

SEQ ID NO: 140 - RNA, 51 nt, synthetic construct; identical to SEQ ID NO: 137
  source 1..51: mol_type = other RNA

SEQ ID NO: 141 - RNA, 51 nt, synthetic construct; identical to SEQ ID NO: 137
  source 1..51: mol_type = other RNA

SEQ ID NO: 142 - RNA, 51 nt, synthetic construct; identical to SEQ ID NO: 136
  source 1..51: mol_type = other RNA

SEQ ID NO: 143 - RNA, 51 nt, synthetic construct; identical to SEQ ID NO: 137
  source 1..51: mol_type = other RNA

SEQ ID NO: 144 - RNA, 51 nt, synthetic construct; identical to SEQ ID NO: 137
  source 1..51: mol_type = other RNA

SEQ ID NO: 145 - RNA, 51 nt, synthetic construct; identical to SEQ ID NO: 136
  source 1..51: mol_type = other RNA

SEQ ID NO: 146 - RNA, 51 nt, synthetic construct; identical to SEQ ID NO: 137
  source 1..51: mol_type = other RNA

SEQ ID NO: 147 - RNA, 51 nt, synthetic construct; identical to SEQ ID NO: 136
  source 1..51: mol_type = other RNA

SEQ ID NO: 148 - RNA, 51 nt, synthetic construct; identical to SEQ ID NO: 136
  source 1..51: mol_type = other RNA

SEQ ID NO: 149 - RNA, 51 nt, synthetic construct
  source 1..51: mol_type = other RNA
atgaaggccaaactgctggtgctgctgtgtacctttacagccacatatgcc

SEQ ID NO: 150 - RNA, 51 nt, synthetic construct
  source 1..51: mol_type = other RNA
atgaaggccaagctcctggtgctgctgtgtaccttcactgccacttacgcc

SEQ ID NO: 151 - RNA, 51 nt, synthetic construct; identical to SEQ ID NO: 132
  source 1..51: mol_type = other RNA

SEQ ID NO: 152 - RNA, 51 nt, synthetic construct
  source 1..51: mol_type = other RNA
atgaaagccaagctgctggtgctgctgtgtacttttactgcaacctacgcc

SEQ ID NO: 153 - RNA, 720 nt, synthetic construct
  source 1..720: mol_type = other RNA
gtggaacccaccaccacaatcagcgccacaagcacacacgagctgagcgcctccgatgccagaaactctatccagctgctgaacgcccacattgccacactgcagagcgtgcagaaaagcgtgccaggcagcgactacagcgaccagatcagagatctgctgaaggccgccttcgacctgagaggcctgattgaaacactggcccacggcggcatcccattctacgatcctagcaccatcatgccccggatcaagctggtggccacaaccatcgataccatccacaccgccaccacaactctgcagaacaaagtgcggcctgctcacgtggaactgggcctcgaagtgacaaaagccgtgctgctgacagccaatcctgccagcacagccaaagagctggacgctgaaggcgccgctctgaaggccagactggaaaaagtgtctcagtaccccgatctgacccctaacgacgtggccacagtgtacgtgcggaccaacttcagcaagaccatctggcaagtgcgggccaacagagatcggtacatcctgggacacaagagcgccgctgtgtacaagaccctgaaccacgccatcacaaaggccgtgggcgtcagactgaaccccaagaccacagtgggcaacatccaggccgccagaacagaactgctggccgcctatcagaccgccttcaacagccccgatgtgaagaaagccgcttaa

SEQ ID NO: 154 - RNA, 720 nt, synthetic construct
  source 1..720: mol_type = other RNA
gtcgagcctacaacaacaatcagcgccacaagcacccacgagctgagcgcctctgatgcccggaactctatccagctgctgaacgcccacatcgccaccctgcagagcgtgcagaagtctgtgccaggcagcgactacagcgaccagatcagagatctgcttaaagccgctttcgacctgagaggcctgatcgagacactggcccacggcggaatccccttctacgaccccagcaccatcatgcctagaatcaagctggtcgccaccaccatcgacaccatccacaccgccacaaccaccctgcagaacaaggtgcggcctgcccatgtggaactgggactggaggtgacaaaagccgtgctgctcaccgccaacccagctagcacagccaaggaactggacgccgagggcgccgccctgaaggccagactggaaaaggtgtcccagtaccccgacctgacccctaacgacgtggccaccgtgtacgtgcgcaccaacttcagcaagaccatttggcaggtgcgggccaatagagatagatacatcctgggccacaagtccgctgccgtgtacaagacactgaatcacgccatcaccaaggctgttggcgtgcggctgaaccctaagaccactgtgggcaacatccaggccgctagaacagagctgctggccgcctatcaaacagcttttaacagccctgatgtgaagaaagccgcttaa

SEQ ID NO: 155 - RNA, 720 nt, synthetic construct
  source 1..720: mol_type = other RNA
gtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacaccaaacgatgtggccactgtgtacgtgagaaccaacttctccaagaccatctggcaggtcagagccaaccgcgatcgctacatcctgggccacaagtccgccgccgtgtataagaccctgaaccatgccatcaccaaggccgtgggcgtcagactgaaccccaagaccaccgtgggcaacatccaggccgctagaacagagctgctggccgcctaccagaccgccttcaattcacccgacgtgaagaaggccgcttaa

SEQ ID NO: 156 - RNA, 717 nt, synthetic construct
  source 1..717: mol_type = other RNA
gtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacccccaacgacgtggccactgtgtacgtgcggaccaacttcagcaagaccatttggcaggtcagggccaatagagacagatacatcctggggcacaaaagcgccgccgtgtacaagaccctgaaccacgccatcaccaaggccgtgggagtgcggctgaatccaaagaccaccgtgggcaacattcaggccgccagaaccgagctgctggccgcctaccagacagctttcaatagccccgacgtgaagaaggccgcc

SEQ ID NO: 157 - RNA, 717 nt, synthetic construct
  source 1..717: mol_type = other RNA
gtggagcccaccacaacaatcagcgccacatccacccacgaactgagcgcctccgacgccagaaattccatccagctgctgaacgcccacatcgccaccctgcagagcgtgcagaaaagcgtgcccggctctgattacagcgaccagatccgggacctgctgaaggccgcctttgacctgagaggcctgatcgagacactggcccacggaggcatccctttctacgatcctagcaccatcatgcctcggatcaagctggtggccacaacaatcgacaccatccacaccgccaccaccacactgcagaacaaagtgcggcctgcccatgtggaactgggcctggaagtgaccaaggccgtgctgctgacagccaatcctgctagcacagccaaggaactggatgccgagggagccgccctgaaggctagactggagaaggtgagccagtaccccgacctgacccccaatgacgtggcaaccgtgtacgtgagaaccaatttcagcaagaccatctggcaggtgcgggccaatcgggacagatacatcctgggccacaagagcgccgccgtgtacaagaccctgaatcacgccatcaccaaggccgtgggcgtgcggctgaatcctaagaccaccgtgggcaatatccaggccgcccggacagaactgctggccgcctaccagacagcctttaatagccctgatgtgaagaaggccgcc

SEQ ID NO: 158 - RNA, 720 nt, synthetic construct
  source 1..720: mol_type = other RNA
gtggaacctaccacaaccatcagcgccaccagcacacacgagctgagcgcttctgatgcccggaacagcatccaactgctgaatgcccacatcgccacactccagagcgtccagaagagcgtccccggcagcgactacagcgatcagatcagagacctgctgaaggccgctttcgacctgagaggcctgatcgagacacttgcccacggcggcatccccttctacgaccccagcaccatcatgcctagaatcaagctggtggccaccaccatcgacaccatccacaccgccacaactaccctgcaaaacaaggtgcgcccagcccatgtggaactgggcctggaggtgaccaaggcagttctgctgacagctaatcctgcctctaccgctaaagagctggacgccgagggcgccgccctgaaagccagactggaaaaggtgtcccagtaccctgatctgacccctaacgacgtggccacagtgtacgtgcggaccaacttcagcaagaccatctggcaggtgcgggccaacagagatagatacatcctgggacacaagtccgccgccgtgtacaagaccctgaaccacgccattaccaaagccgtgggcgtgcggctgaaccctaagacaaccgtgggaaatatccaggccgctagaaccgagctgctggccgcttatcagacagcctttaacagccctgacgtgaagaaggccgcctga

SEQ ID NO: 159 - RNA, 720 nt, synthetic construct
  source 1..720: mol_type = other RNA
gtcgagcctaccacaaccatcagcgccacatctacacacgagctgagcgccagcgacgccagaaacagcatccagctgctgaacgcccacatcgccacactgcagagcgtgcagaaatctgtgcccggctccgattacagcgatcagatcagagacctgcttaaagccgctttcgacctgagaggactgatcgagacactggcccacggaggcatccccttctacgaccccagcaccatcatgcctagaatcaagctggtggccaccaccatcgacactatccacaccgctaccacaaccctgcaaaacaaggtgcggcctgcccatgtggaactgggcctggaagtgaccaaagccgtgctgctgaccgccaaccctgcttctaccgccaaggaactggacgccgagggcgccgctctgaaagcgcggctggagaaggtttctcagtaccctgatctgacccctaacgacgtggccacagtgtacgtgaggaccagcttcagcaagaccatctggcaggtgcgggccaatagagatagatacatcctgggccacaagtccgccgccgtgtataagacactgaaccacgccatcaccaaggctgtcggagtgcggctgaatccaaagaccaccgtgggcaatatccaagccgctagaaccgagctgctcgccgcctaccagacagcttttaacagccctgacgtgaagaaggccgcctga

SEQ ID NO: 160 - RNA, 717 nt, synthetic construct
  source 1..717: mol_type = other RNA
gtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacccccaacgatgtggccaccgtgtatgtgcggacctccttctccaagaccatctggcaggtcagagccaatcgggaccggtacatcctgggccacaagagcgccgctgtgtacaagaccctgaaccatgccatcaccaaggcagtgggcgtgcgcctgaacccaaaaaccaccgtgggcaacattcaggccgccagaaccgagctgctggccgcctaccagaccgctttcaatagccccgacgtgaagaaggccgcc

SEQ ID NO: 161 - RNA, 720 nt, synthetic construct; identical to SEQ ID NO: 153
  source 1..720: mol_type = other RNA

SEQ ID NO: 162 - RNA, 720 nt, synthetic construct; identical to SEQ ID NO: 154
  source 1..720: mol_type = other RNA

SEQ ID NO: 163 - RNA, 720 nt, synthetic construct
  source 1..720: mol_type = other RNA
gtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgaccccaaatgacgtggccaccgtgtacgtgcggacctccttctccaagaccatctggcaagtgcgggctaacagagacaggtacattctgggccacaagtccgccgccgtgtacaaaaccctgaatcacgccatcaccaaggccgtgggcgtgagactgaaccccaagaccaccgtgggcaacattcaggctgccaggaccgagctgctggccgcttatcagaccgccttcaacagccccgacgtgaagaaagccgcctaa

SEQ ID NO: 164 - RNA, 720 nt, synthetic construct
  source 1..720: mol_type = other RNA
gtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccggcgggacacctaacggcgtgggaggcgtgtatgtgaggacaaacttcagcaagacaatctggcaggtcagggccaaccgcgatagatacatcctgggccacaagagcgccgccgtctacaagacactgaaccacgctatcacaaaagccgtgggggtgaggctgaaccccaagaccaccgtggggaacatccaggccgctagaactgagctgctggccgcctaccagaccgctttcaatagccccgacgtgaagaaggccgcctaa

SEQ ID NO: 165 - RNA, 720 nt, synthetic construct
  source 1..720: mol_type = other RNA
gtggaacctacaaccacaatcagcgccacatctacacacgagctgagcgccagcgacgcccggaacagcatccagctgctgaacgcccacatcgccaccctccagagcgtgcagaaatccgtgcctggatctgattacagcgaccagatcagagatctgctgaaggccgcttttgacctgagaggcctgatcgagacactggcccatggcggcatccccttctacgaccctagcacaattatgcctagaatcaagctggtcgccaccaccatcgacaccatccacaccgccaccacaaccctgcagaacaaggtcagaccagcccacgtggaactcggcctggaggtgaccaaggctgtgctgcttacagctaatcctgctagcaccgctaaagagctggacgccgagggcgccgccctgaaggcccgcctggaaaaagtgtcccagtaccccggcggaacccctaacggcgttggcggagtgtacgtgcggaccagcttcagcaagaccatctggcaggtgcgggctaatagagatagatacatcctgggccacaagtccgccgcggtgtataagacactgaaccacgccatcaccaaggctgtgggcgtgcggctgaaccccaagaccacagtgggcaacatccaagccgccagaaccgagctgctggccgcctaccagaccgccttcaactctccagacgtgaagaaggccgcctga

SEQ ID NO: 166 - RNA, 720 nt, synthetic construct
  source 1..720: mol_type = other RNA
gtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacccccaacgatgtggctaccgtctatgtgcggactaatttcagtaagaccatctggcaggtcgccgccaacgccgatagatatatcgccggccacaaatccgccgccgtcgcaaagacccttgctcacgctattgccaaggccgctggcgtgagagctaacccaaagaccaccgtcggcaacatccaggccgccaggacagagctgcttgccgcctatcagactgccttcaacagccctgacgtgaagaaggcagcctaa

SEQ ID NO: 167 - RNA, 720 nt, synthetic construct
  source 1..720: mol_type = other RNA
gtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacccccaacgacgtggccaccgtgtatgtgagaaccagcttcagcaaaacaatctggcaggtcgccgccaacgccgacagatacatcgcaggccacaagagtgctgccgtggcgaaaaccctggcacacgcaattgccaaggccgcaggcgtgcgggccaatcctaagaccactgtgggcaatatccaggccgccaggactgaactgctggctgcctaccagacagccttcaacagcccagacgtgaagaaggccgcctaa

SEQ ID NO: 168 - RNA, 897 nt, synthetic construct
  source 1..897: mol_type = other RNA
agctccaaccggccaagaagcgtcgcccaggcagccatcgcaacagacggcaaggggatcatcaataaggactgcagagacgccgtgatcaatgatgccaaactgagggccgccatcgccggagctctggtgaaggccgggttttctagcgccgacgctgtggcactggcacctagaatcgctcgggaaatggccaaggagggggttctgctgattaaccaccacaagctgaaagcactgatcggagcccaggtgggactgctgacagatgccaagatccagagagccgcagctgcagtggacctgggcatcaaagccacactggccgcaaccattatccccaatgccctgcactccgcagccttcaaggatgctgtggtggccaacctggtggcagctggggttgataagaagctggccaaggccaccgcagttgccattgctgccacagccctgaatcctgcactgggacccatcgcaaagactgaggcaatcaaggccgagattggcgcacaggccgctctgctggtgagcagaggagtgcacctgaagaaggccgccattgagcacatcattgggagatcctttgacgccgcagtggccaccgccattgttagctcccccatcctgaatgcccggatcgtgacccacctcgtgcgggcaggaattgacaagagcctcgcagtgcagattgccccacggattatagatagactggccaaggagccgctgctggccctgaacactgcaaagctgatgaaaaacatcacaaggcagatcgtcgacgtgattaccgccgacaaggcaattaagaccgccgagcagctggagaaggagctgcctgccctggacgacctggtgaagaaggcctgctcctcccccaagcctactccaacc

SEQ ID NO: 169 - RNA, 879 nt, synthetic construct
  source 1..879: mol_type = other RNA
tccagcaaccggcctagaagcgtggcccaggctgccatcgccacagatggcaagggcatcatcaacaaggactgtagagacgccgtgatcaatgacgccaagctgagagccgccatcgccggagccctggtgaaggccggctttagcagcgccgacgccgtggccctggcccctcggatcgcccgggagatggccaaggagggagtgctgctgatcaatcatcacaagctgaaggccctgattggcgcccaggtgggactgctgaccgacgccaagattcagagagccgccgctgccgtggatctgggcatcaaggccaccctggccgccaccatcatccctaacgccctgcactccgccgcttttaaggatgccgtggtggctaacctggtggctgccggcgtggacaagaagctggccaaagctaccgccgttgccatcgccgctacagctctgaatcctgccctggggcccatcgccaagacagaggccattaaggccgaaattggagcccaggccgcactgctggtgtccagaggcgtgcacctgaagaaggccgccatcgagcacatcattggcagatccttcgatgctgccgtggctaccgccatcgtgagcagccctatcctgaatgccagaatcgtgacccacctggtgagagcaggcatcgacaagagcctggccgtgcagatcgcccctcggattatcgaccggctggccaaggagcccctgctggccctgaacaccgccaagctgatgaagaacatcaccagacagatcgtggatgtgatcaccgccgataaggccatcaaaacagccgagcagctggagaaggagctgcctgccctggatgacctggtgaagaaggcctgtagctaa

SEQ ID NO: 170 - RNA, 876 nt, synthetic construct
  source 1..876: mol_type = other RNA
agctccaaccggccaagaagcgtcgcccaggcagccatcgcaacagacggcaaggggatcatcaataaggactgcagagacgccgtgatcaatgatgccaaactgagggccgccatcgccggagctctggtgaaggccgggttttctagcgccgacgctgtggcactggcacctagaatcgctcgggaaatggccaaggagggggttctgctgattaaccaccacaagctgaaagcactgatcggagcccaggtgggactgctgacagatgccaagatccagagagccgcagctgcagtggacctgggcatcaaagccacactggccgcaaccattatccccaatgccctgcactccgcagccttcaaggatgctgtggtggccaacctggtggcagctggggttgataagaagctggccaaggccaccgcagttgccattgctgccacagccctgaatcctgcactgggacccatcgcaaagactgaggcaatcaaggccgagattggcgcacaggccgctctgctggtgagcagaggagtgcacctgaagaaggccgccattgagcacatcattgggagatcctttgacgccgcagtggccaccgccattgttagctcccccatcctgaatgcccggatcgtgacccacctcgtgcgggcaggaattgacaagagcctcgcagtgcagattgccccacggattatagatagactggccaaggagccgctgctggccctgaacactgcaaagctgatgaaaaacatcacaaggcagatcgtcgacgtgattaccgccgacaaggcaattaagaccgccgagcagctggagaaggagctgcctgccctggacgacctggtgaagaaggcctgctcc

SEQ ID NO: 171 - RNA, 897 nt, synthetic construct
  source 1..897: mol_type = other RNA
gccagcaatggcaacagctccatcacccagagcgccgccttcagccctagagctaccacaaagatcagcgaagattgcagaaaggccattatcaatgatctgaaactgagaggcgccatcgtgggagctctggtgaaggccggcctgtctgccgccgacgccgccgccctggcccctagaattgccgccgaaatggccgccgaaggcaccctgaccatcaatcaccaccggctgaaggtgctggtcgcctctcaactgggcctggtggccgatgctgcagtgcaacatgccgcagccgccatcgacctgagcttcaaggccatcctgggcgccagcatcatccctaatgccctgggctccgccgctttcaagaatgccgtgatcgctaatctcgtggccgccggcattgacaagcacctggccagagccacagcagtggccatcgttgccacagcactgaatcccgctctgggtcctatcgccaagttcgagctgatcaaggccgagatcgccgcccaggccgccctgctgatcagaagaggcgtgcatctgcagaaagctgccatcgagcacgtgatcggacgggcattcgacgctgccgtggccacagccatcatcagcagccctatcctgtctgccagaatcgtgacacacctggtgagagccggcattgacaaatctatcgccatcagcctggcccctcacatcgtgaagagactggccaaggagcccctgctggccttcaatacagccaagctggtgaaggatatcgccagacagattgtggacatccggaatacccaggaggccatcgccgtgtacaagcagctgaaggccgagctgcctaccctggatggcctggtgcagaaggcttgcaccccagaacctacacccacc

SEQ ID NO: 172 - RNA, 879 nt, synthetic construct
  source 1..879: mol_type = other RNA
gccagcaatggcaacagctccatcacccagagcgccgccttcagccctagagctaccacaaagatcagcgaagattgcagaaaggccattatcaatgatctgaaactgagaggcgccatcgtgggagctctggtgaaggccggcctgtctgccgccgacgccgccgccctggcccctagaattgccgccgaaatggccgccgaaggcaccctgaccatcaatcaccaccggctgaaggtgctggtcgcctctcaactgggcctggtggccgatgctgcagtgcaacatgccgcagccgccatcgacctgagcttcaaggccatcctgggcgccagcatcatccctaatgccctgggctccgccgctttcaagaatgccgtgatcgctaatctcgtggccgccggcattgacaagcacctggccagagccacagcagtggccatcgttgccacagcactgaatcccgctctgggtcctatcgccaagttcgagctgatcaaggccgagatcgccgcccaggccgccctgctgatcagaagaggcgtgcatctgcagaaagctgccatcgagcacgtgatcggacgggcattcgacgctgccgtggccacagccatcatcagcagccctatcctgtctgccagaatcgtgacacacctggtgagagccggcattgacaaatctatcgccatcagcctggcccctcacatcgtgaagagactggccaaggagcccctgctggccttcaatacagccaagctggtgaaggatatcgccagacagattgtggacatccggaatacccaggaggccatcgccgtgtacaagcagctgaaggccgagctgcctaccctggatggcctggtgcagaaggcttgcacc

SEQ ID NO: 173 - RNA, 882 nt, synthetic construct
  source 1..882: mol_type = other RNA
gctagcaatggaaactcctctattacacagtctgccgccttcagcccaagagctaccacaaagatcagcgaggattgtagaaaggccatcattaacgatctgaagctgcggggcgccatcgtgggcgctctcgtgaaagccggactgtctgcagcagacgctgcagcactggctccaaggatcgccgctgagatggcagcagaggggacactgactattaaccaccataggctgaaagtgctggtcgcttcccagctgggcctggttgcagatgcagccgtgcagcatgctgccgctgcaattgacctgagcttcaaagctatcctgggcgcctctatcattccaaatgctctcgggagcgcagccttcaaaaacgccgtgattgccaacctggtggctgcaggaattgacaagcacctggccagagccactgctgtggcaattgtggccactgctctgaatccagcactgggacccatcgcaaagtttgagctgattaaggcagaaattgccgctcaagcagccctgctgattagacggggagtgcacctgcagaaggccgcaattgaacacgtgatcggccgggcatttgacgccgctgttgctactgccatcatcagcagccctattctgtccgccaggattgtcacccacctggtcagggcaggcattgataagagcattgctatcagcctggccccacacatcgttaagcggctggccaaagaacccctgctggcttttaatactgctaagctggtgaaagacattgcccgccagatcgtggacattaggaatactcaggaggccatcgccgtgtataaacagctcaaggcagaactgcctaccctggacggcctggtgcagaaagcctgtacataa

SEQ ID NO: 174 - RNA, 882 nt, synthetic construct
  source 1..882: mol_type = other RNA
gctagcaatggaaactcctctattacacagtctgccgccttcagcccaagagctaccacaaagatcagcgaggattgtagaaaggccatcattaacgatctgaagctgcggggcgccatcgtgggcgctctcgtgaaagccggactgtctgtggcagacgctgcagcactggctccaaggatcgccgctgagatggcagcagaggggacactgactattaaccaccataggctgaaagtgctggtcgcttcccagctgggcctggttgcagatgcagccgtgcagcatgctgccgctgcaattgacctgagcttcaaagctatcctgggcgcctctatcattccaaatgctctcgggagcgcagccttcaaaaacgccgtgattgccaacctggtggctgcaggaattgacaagcacctggccagagccactgctgtggcaattgtggccactgctctgaatccagcactgggacccatcgcaaagtttgagctgattaaggcagaaattgccgctcaagcagccctgctgattagacggggagtgcacctgcagaaggccgcaattgaacacgtgatcggccgggcatttgacgccgctgttgctactgccatcatcagcagccctattctgtccgccaggattgtcacccacctggtcagggcaggcattgataagagcattgctatcagcctggccccacacatcgttaagcggctggccaaagaacccctgctggcttttaatactgctaagctggtgaaagacattgcccgccagatcgtggacattaggaatactcaggaggccatcgccgtgtataaacagctcaaggcagaactgcctaccctggacggcctggtgcagaaagcctgtacataa

SEQ ID NO: 175 - RNA, 897 nt, synthetic construct
  source 1..897: mol_type = other RNA
gccagcaatggcaacagctccatcacccagagcgccgccttcagccctagagctaccacaaagatcagcgaagattgcagaaaggccattatcaatgatctgaaactgagaggcgccatcgtgggagctctggtgaaggccggcctgtctgtggccgacgccgccgccctggcccctagaattgccgccgaaatggccgccgaaggcaccctgaccatcaatcaccaccggctgaaggtgctggtcgcctctcaactgggcctggtggccgatgctgcagtgcaacatgccgcagccgccatcgacctgagcttcaaggccatcctgggcgccagcatcatccctaatgccctgggctccgccgctttcaagaatgccgtgatcgctaatctcgtggccgccggcattgacaagcacctggccagagccacagcagtggccatcgttgccacagcactgaatcccgctctgggtcctatcgccaagttcgagctgatcaaggccgagatcgccgcccaggccgccctgctgatcagaagaggcgtgcatctgcagaaagctgccatcgagcacgtgatcggacgggcattcgacgctgccgtggccacagccatcatcagcagccctatcctgtctgccagaatcgtgacacacctggtgagagccggcattgacaaatctatcgccatcagcctggcccctcacatcgtgaagagactggccaaggagcccctgctggccttcaatacagccaagctggtgaaggatatcgccagacagattgtggacatccggaatacccaggaggccatcgccgtgtacaagcagctgaaggccgagctgcctaccctggatggcctggtgcagaaggcttgcaccccagaacctacacccacc

SEQ ID NO: 176 - RNA, 879 nt, synthetic construct
  source 1..879: mol_type = other RNA
gctagcaatggaaactcctctattacacagtctgccgccttcagcccaagagctaccacaaagatcagcgaggattgtagaaaggccatcattaacgatctgaagctgcggggcgccatcgtgggcgctctcgtgaaagccggactgtctgtggcagacgctgcagcactggctccaaggatcgccgctgagatggcagcagaggggacactgactattaaccaccataggctgaaagtgctggtcgcttcccagctgggcctggttgcagatgcagccgtgcagcatgctgccgctgcaattgacctgagcttcaaagctatcctgggcgcctctatcattccaaatgctctcgggagcgcagccttcaaaaacgccgtgattgccaacctggtggctgcaggaattgacaagcacctggccagagccactgctgtggcaattgtggccactgctctgaatccagcactgggacccatcgcaaagtttgagctgattaaggcagaaattgccgctcaagcagccctgctgattagacggggagtgcacctgcagaaggccgcaattgaacacgtgatcggccgggcatttgacgccgctgttgctactgccatcatcagcagccctattctgtccgccaggattgtcacccacctggtcagggcaggcattgataagagcattgctatcagcctggccccacacatcgttaagcggctggccaaagaacccctgctggcttttaatactgctaagctggtgaaagacattgcccgccagatcgtggacattaggaatactcaggaggccatcgccgtgtataaacagctcaaggcagaactgcctaccctggacggcctggtgcagaaagcctgtaca

SEQ ID NO: 177 - RNA, 879 nt, synthetic construct
  source 1..879: mol_type = other RNA
gctagcaatggacagtcctctattacacagtctgccgccttcagcccaagagctaccacaaagatcagcgaggattgtagaaaggccatcattaacgatctgaagctgcggggcgccatcgtgggcgctctcgtgaaagccggactgtctgcagcagacgctgcagcactggctccaaggatcgccgctgagatggcagcagaggggacactgactattaaccaccataggctgaaagtgctggtcgcttcccagctgggcctggttgcagatgcagccgtgcagcatgctgccgctgcaattgacctgagcttcaaagctatcctgggcgcctctatcattccaaatgctctcgggagcgcagccttcaaaaacgccgtgattgccaacctggtggctgcaggaattgacaagcacctggccagagccactgctgtggcaattgtggccactgctctgaatccagcactgggacccatcgcaaagtttgagctgattaaggcagaaattgccgctcaagcagccctgctgattagacggggagtgcacctgcagaaggccgcaattgaacacgtgatcggccgggcatttgacgccgctgttgctactgccatcatcagcagccctattctgtccgccaggattgtcacccacctggtcagggcaggcattgataagagcattgctatcagcctggccccacacatcgttaagcggctggccaaagaacccctgctggcttttaatactgctaagctggtgaaagacattgcccgccagatcgtggacattaggaatactcaggaggccatcgccgtgtataaacagctcaaggcagaactgcctaccctggacggcctggtgcagaaagcctgtaca

SEQ ID NO: 178 - RNA, 897 nt, synthetic construct
  source 1..897: mol_type = other RNA
gctagcaatggacagtcctctattacacagtctgccgccttcagcccaagagctaccacaaagatcagcgaggattgtagaaaggccatcattaacgatctgaagctgcggggcgccatcgtgggcgctctcgtgaaagccggactgtctgcagcagacgctgcagcactggctccaaggatcgccgctgagatggcagcagaggggacactgactattaaccaccataggctgaaagtgctggtcgcttcccagctgggcctggttgcagatgcagccgtgcagcatgctgccgctgcaattgacctgagcttcaaagctatcctgggcgcctctatcattccaaatgctctcgggagcgcagccttcaaaaacgccgtgattgccaacctggtggctgcaggaattgacaagcacctggccagagccactgctgtggcaattgtggccactgctctgaatccagcactgggacccatcgcaaagtttgagctgattaaggcagaaattgccgctcaagcagccctgctgattagacggggagtgcacctgcagaaggccgcaattgaacacgtgatcggccgggcatttgacgccgctgttgctactgccatcatcagcagccctattctgtccgccaggattgtcacccacctggtcagggcaggcattgataagagcattgctatcagcctggccccacacatcgttaagcggctggccaaagaacccctgctggcttttaatactgctaagctggtgaaagacattgcccgccagatcgtggacattaggaatactcaggaggccatcgccgtgtataaacagctcaaggcagaactgcctaccctggacggcctggtgcagaaagcctgtacacccgagcctacacctacc

SEQ ID NO: 179 - RNA, 918 nt, synthetic construct
  source 1..918: mol_type = other RNA
tcatccaacaggcctcgctccgtggcccaggctgctattgctaccgacggcaagggaatcatcgataaagactccagggatgccgtgatcaacgatgccaagctccgggccgcaattgccggagccctggtgaaagccgggttcagctccgccgatgctgtggctctggctccaaggattgctaaggagatggccaaagagggggtgctgctgattaaccaccacaagctgaaggcactgatcggagcacagctgggactgctgactgacgccaaaatccagcgcgccgctgcagcagtcgacctgggcatcaaggccacactggctgctaccatcattcctaacgctctgggcagcgcagcctttaagaatgccgttatcgccaatctcgtggctgctggaattgataagcacctggccagagccacagctgtggccatcgtggccactgccctgaacccagcattgggccctatcgctaagtttgagctgattaaggccgagatcgccgctcaggctgccctgctgattcgcaggggagtgcacctgcagaaggctgccatcgagcacgtcatcggcagagcattcgacgccgctgtcgccactgctatcatctctagcccaatcctgagcgccaggatcgtcactcacctggtgagagctgggattgacaaatccatcgctatcagcctggcccctcacatcgtgaagaggctggctaaggaaccactgctggcttttaacactgctaagctgatgaagaatattacaagacagatcgtggacgtcatcaccgccgacaaagccatcaagacagccgagcagctcgagaaggagctgcctgccctggatgacctggtgaagaaggcatcctccccccctaagccaacccctaccccaacccctacacctacttaa

SEQ ID NO: 180 - skipped

SEQ ID NO: 181 - RNA, 918 nt, synthetic construct
  source 1..918: mol_type = other RNA
tcatccaacaggcctcgctccgtggcccaggctgctattgctaccgacggcaagggaatcatcgataaagactccagggatgccgtgatcaacgatgccaagctccgggccgcaattgccggagccctggtgaaagccgggttcagctccgccgatgctgtggctctggctccaaggattgctaaggagatggccaaagagggggtgctgctgattaaccaccacaagctgaaggcactgatcggagcacagctgggactgctgactgacgccaaaatccagcgcgccgctgcagcagtcgacctgggcatcaaggccacactggctgctaccatcattcctaacgctctgggcagcgcagcctttaagaatgccgttatcgccaatctcgtggctgctggaattgataagcacctggccagagccacagctgtggccatcgtggccactgccctgaacccagcattgggccctatcgctaagtttgagctgattaaggccgagatcgccgctcaggctgccctgctgattcgcaggggagtgcacctgcagaaggctgccatcgagcacgtcatcggcagagcattcgacgccgctgtcgccactgctatcatctctagcccaatcctgagcgccaggatcgtcactcacctggtgagagctgggattgacaaatccatcgctatcagcctggcccctcacatcgtgaagaggctggctaaggaaccactgctggcttttaacactgctaagctgatgaagcagattacaagacagatcgtggacgtcatcaccgccgacaaagccatcaagacagccgagcagctcgagaaggagctgcctgccctggatgacctggtgaagaaggcatcctccccccctaagccaacccctaccccaacccctacacctacttaa

SEQ ID NO: 182 - RNA, 1200 nt, synthetic construct
  source 1..1200: mol_type = other RNA
gcaggccctaccgtcacagtgatccctgtgggcagagaaggtggcgacatcaccatcagcggcaagggcttcagcaccacaggctttggcgtgtacgtggccgtggcacctgctagcgtgcccgaattctacggcaatagcgacaagttctacggctacgaccccagcaaggacaccaccgagagccctagcaccatctgggtgtacacccctagccagaaggccatcggctctcggttcgcccagggcagacccatgaacaatgacggcagcttcacaatcaccatgaaggccccccctttcgagcagggcaaagacttcgtggtgctgaccacaaaggcccatggcgtgggcaaaaccgatcacagcgacgacacacggacacctgtcacctacagagaggctacccctgcccctacaggccctaagacacccatcgcccctagcaagcagcctagcaagcaggccgcccctagcaagcaggtgaagccttctaagcaggccggccccaacaagcagtccaccacacctcagcagaagacagccgagcacagaagccagacccctgccgcccaccggaccatgacaaagcaggtgagcaccatcggcgccagcaaggtgacaagcggctctctgacctggggcatcagaacctcttttacctcctacctgagaggccctatcgccaacggctcttggaagctgagcggaggcgccaattggaatggctccgccttcaccttccctctgaccagcggcagcttcgacccagccaccaagtccggaagcctgaagtacagcggcagcgtgcacatgacaggccaccacggcatcctggacatgaccctggccgagccaagcctgcagatcaagggaagcaccggccacctgtatctggacgtgaaaagcagcagcatggacggcaagaagaccaattacggcagagtggacttcgccaccttcggagtgagcgtgtctggcaacgccgccatcaagggcagccctgtgaagctgaccgccacaggcgctaaggcttttgccggcttctacagagccggcgagcctatgaaccctctgagcaccaatctgaccctgagcgccgagaaggtgtcccacaacgtgaccgtggacgcagtgacaggcaaggtgatcggcgacgactctggcaagggagccggccggggcctgcccgtgacataa

SEQ ID NO: 183 - skipped

SEQ ID NO: 184 - RNA, 1200 nt, synthetic construct
  source 1..1200: mol_type = other RNA
gctgggccaacagtgacagtgatccccgtgggacgcgagggcggagacattaccatctccgggaagggctttagcacaacagggttcggtgtgtatgtggctgtggccccagcaagcgtgccagagttctacgggaactccgataaattttatgggtatgaccccagtaaagacacaaccgagagccccagcacaatttgggtttacaccccctctcagaaagctattggctcccggttcgcccagggcagaccaatgaataatgacgggagctttaccatcactatgaaggcccccccattcgagcagggaaaggactttgtggtcctgactaccaaggctcacggagtgggcaaaaccgaccactctgacgacactcggacaccggtgacataccgggaggctactcccgctccaacaggccctaaaactccaatcgcccccagcaagcagccttcaaagcaggctgcccccagcaagcaggtgaagcctagcaagcaggctggccccaacaagcagagcactactcctcagcagaagaccgcagagcacagatctcagaccccagcagctcatcgcactatgactaagcaggtgtgtaccatcggagcctcaaaggtgaccagcggatccctgacatggggaatccggactagctttacttcctacctgcgcgggcccatcgcaaacggcagctggaagctgtctgggggggccaattggaacggcagcgccttcacattcccactgacttctggatccttcgaccccgccaccaaatccggctcactgaagtacagcgggagcgtgcacatgactggacaccatggaattctggacatgacactcgctgaaccctctctgcagatcaagggctctactgggcacctgtatctggatgtgaagtcctcttctatggacggcaagaagacaaactatgggagagtggattttgccaccttcggagtgagcgtgagcggaaatgctgcaatcaaagggtcccccgtgaaactgaccgctaccggagcaaaagcctttgccggcttttacagagcaggagagcccatgaaccccctgagcaccaatctgacactgagcgctgagaaagtttgtcacaatgtgaccgttgatgcagttaccgggaaagtgatcggcgacgattccggaaagggggcaggaaggggactgcctgtcacttaa

SEQ ID NO: 185 - RNA, 1200 nt, synthetic construct
  source 1..1200: mol_type = other RNA
gctgggccaacagtgacagtgatccccgtgggacgcgagggcggagacattaccatctccgggaagggctttagcacaacagggttcggtgtgtatgtggctgtggccccagcaagcgtgccagagttctacgggaactccgataaattttatgggtatgaccccagtaaagacacaaccgagagccccagcacaatttgggtttacaccccctctcagaaagctattggctcccggttcgcccagggcagaccaatgaataatgacgggagctttaccatcactatgaaggcccccccattcgagcagggaaaggactttgtggtcctgactaccaaggctcacggagtgggcaaaaccgaccactctgacgacactcggacaccggtgacataccgggaggctactcccgctccaacaggccctaaaactccaatcgcccccagcaagcagccttcaaagcaggctgcccccagcaagcaggtgaagcctagcaagcaggctggccccaacaagcagagcactactcctcagcagaagaccgcagagcacagatctcagaccccagcagctcatcgcactatgactaagcaggtgtgtaccatcggagcctcaaaggtgaccagcggatccctgacatggggaatccggactagctttacttcctacctgcgcgggcccatcgcaaagggcagctggaagctgtctgggggggccaattggaacggaggcgccttcacattcccactgacttctggatccttcgaccccgccaccaaatccggctcactgaagtacagcgggagcgtgcacatgactggacaccatggaattctggacatgacactcgctgaaccctctctgcagatcaagggctctactgggcacctgtatctggatgtgaagtcctcttctatggacggcaagaagacaaactatgggagagtggattttgccaccttcggagtgagcgtgagcggaaatgctgcaatcaaagggtcccccgtgaaactgaccgctaccggagcaaaagcctttgccggcttttacagagcaggagagcccatgaaccccctgagcacccagctgacactgagcgctgagaaagtttgtcacagcgtgaccgttgatgcagttaccgggaaagtgatcggcgacgattccggaaagggggcaggaaggggactgcctgtcacttaa

SEQ ID NO: 186 - RNA, 1200 nt, synthetic construct
  source 1..1200: mol_type = other RNA
gcaggccctaccgtcacagtgatccctgtgggcagagaaggtggcgacatcaccatcagcggcaagggcttcagcaccacaggctttggcgtgtacgtggccgtggcacctgctagcgtgcccgaattctacggcaatagcgacaagttctacggctacgaccccagcaaggacaccaccgagagccctagcaccatctgggtgtacacccctagccagaaggccatcggctctcggttcgcccagggcagacccatgaacaatgacggcagcttcacaatcaccatgaaggccccccctttcgagcagggcaaagacttcgtggtgctgaccacaaaggcccatggcgtgggcaaaaccgatcacagcgacgacacacggacacctgtcacctacagagaggctacccctgcccctacaggccctaagacacccatcgcccctagcaagcagcctagcaagcaggccgcccctagcaagcaggtgaagccttctaagcaggccggccccaacaagcagtccaccacacctcagcagaagacagccgagcacagaagccagacccctgccgcccaccggaccatgacaaagcaggtgagcaccatcggcgccagcaaggtgacaagcggctctctgacctggggcatcagaacctcttttacctcctacctgagaggccctatcgccaagggctcttggaagctgagcggaggcgccaattggaatggcggcgccttcaccttccctctgaccagcggcagcttcgacccagccaccaagtccggaagcctgaagtacagcggcagcgtgcacatgacaggccaccacggcatcctggacatgaccctggccgagccaagcctgcagatcaagggaagcaccggccacctgtatctggacgtgaaaagcagcagcatggacggcaagaagaccaattacggcagagtggacttcgccaccttcggagtgagcgtgtctggcaacgccgccatcaagggcagccctgtgaagctgaccgccacaggcgctaaggcttttgccggcttctacagagccggcgagcctatgaaccctctgagcacccagctgaccctgagcgccgagaaggtgtcccacagcgtgaccgtggacgcagtgacaggcaaggtgatcggcgacgactctggcaagggagccggccggggcctgcccgtgacataa

SEQ ID NO: 187 - RNA, 1197 nt, synthetic construct
  source 1..1197: mol_type = other RNA
gccggtccaacagtgacagtgatccctgtgggcagagaaggcggcgatatcacaatcagcggcaagggcttctctacaacaggattcggagtgtatgtggctgtcgcccctgccagcgtgcccgaattctatggaaacagcgacaagttctacggctacgaccccagcaaggatacaaccgagagcccctccaccatctgggtgtacacccccagccagaaggccatcggcagcagattcgcccagggcagacctatgaataatgacggctccttcaccatcaccatgaaggcccctccattcgagcagggcaaggatttcgtggtgctgaccaccaaagcccacggcgtgggcaaaacagaccacagcgatgatacccggacccccgtgacatacagagaagccacaccagcccctaccggccctaagacccctatcgccccttctaagcagcctagcaagcaggccgccccatccaaacaggtgaagcctagcaagcaggccgggcccaataagcagagcacaaccccccagcagaagaccgccgaacacagaagccagacacccgccgcccacagaacaatgaccaagcaggtgtgcaccatcggcgcctccaaggtgaccagcggcagcctgacctggggcattagaaccagctttaccagctacctgagaggccctatcgccaacggcagctggaagctgtccggcggcgccaattggaacggcagcgcctttaccttccccctgacaagcggcagcttcgaccctgccaccaagtctggcagcctgaagtacagcggcagcgtgcatatgaccggccaccacggcatcctggatatgaccctggccgaacctagcctgcagatcaagggcagcaccggccacctgtacctggacgtgaaaagcagcagcatggacggcaagaagacaaattacggcagagtggacttcgccaccttcggcgtgagcgtgagcggaaatgccgccatcaaaggaagccctgtgaagctgaccgccaccggcgccaaggcctttgccggcttctacagagccggcgagcctatgaatcctctgagcaccaatctgaccctgagcgccgaaaaggtgtgccacaatgtgacagtcgacgccgtgacaggcaaggtcatcggcgatgactctggcaaaggagccggcagaggcctgcccgtgaca

SEQ ID NO: 188 - RNA, 1197 nt, synthetic construct
  source 1..1197: mol_type = other RNA
gctgggccaacagtgacagtgatccccgtgggacgcgagggcggagacattaccatctccgggaagggctttagcacaacagggttcggtgtgtatgtggctgtggccccagcaagcgtgccagagttctacgggaactccgataaattttatgggtatgaccccagtaaagacacaaccgagagccccagcacaatttgggtttacaccccctctcagaaagctattggctcccggttcgcccagggcagaccaatgaataatgacgggagctttaccatcactatgaaggcccccccattcgagcagggaaaggactttgtggtcctgactaccaaggctcacggagtgggcaaaaccgaccactctgacgacactcggacaccggtgacataccgggaggctactcccgctccaacaggccctaaaactccaatcgcccccagcaagcagccttcaaagcaggctgcccccagcaagcaggtgaagcctagcaagcaggctggccccaacaagcagagcactactcctcagcagaagaccgcagagcacagatctcagaccccagcagctcatcgcactatgactaagcaggtgtgtaccatcggagcctcaaaggtgaccagcggatccctgacatggggaatccggactagctttacttcctacctgcgcgggcccatcgcaaagggcagctggaagctgtctgggggggccaattggaacggaggcgccttcacattcccactgacttctggatccttcgaccccgccaccaaatccggctcactgaagtacagcgggagcgtgcacatgactggacaccatggaattctggacatgacactcgctgaaccctctctgcagatcaagggctctactgggcacctgtatctggatgtgaagtcctcttctatggacggcaagaagacaaactatgggagagtggattttgccaccttcggagtgagcgtgagcggaaatgctgcaatcaaagggtcccccgtgaaactgaccgctaccggagcaaaagcctttgccggcttttacagagcaggagagcccatgaaccccctgagcacccagctgacactgagcgctgagaaagtttgtcacagcgtgaccgttgatgcagttaccgggaaagtgatcggcgacgattccggaaagggggcaggaaggggactgcctgtcact

SEQ ID NO: 189 - RNA, 1359 nt, synthetic construct
  source 1..1359: mol_type = other RNA
tcctctaatagaccaagatccgtggctcaggccgccatcgctacagatgggaaaggaatcatcgataaggatagtcgggatgcagtgatcaacgacgccaaactccgagctgccatcgccggcgccctggtgaaggctgggttttccagcgccgacgcagtggccctggcacctagaatcgccaaggagatggcaaaggagggcgtcctgctgattaatcaccacaagctgaaagctctgattggggctcagctcggactgctgactgacgccaagatccagagggccgcagctgccgtggatctggggatcaaggccactctggccgctactattattcctaacgctctggggtctgccgccttcaagaatgctgtcattgccaacctggtggctgctggcatcgacaaacacctcgccagggctaccgctgtggccatcgttgccacagccctgaatcctgccctgggcccaatcgctaaatttgagctgattaaggctgagatcgccgcccaggccgcactgctgatcagaaggggggtccatctgcagaaggccgctattgaacatgtgatcggccgcgcattcgacgctgccgtggccaccgctatcatttcctctccaatcctgagcgcacggattgtgacccacctggtgagggctggaattgataaaagcatcgctatctccctggctcctcatatcgtgaagcgcctggccaaggagcccctgctggctttcaacaccgccaagctgatgaagaatattacaagacagatcgttgacgtgatcacagccgataaagccatcaaaacagctgagcagctggaaaaggagctccccgccctggacgacctggtcaaaaaagctagctcccctcctaagcctacccccacacctactccaacccccaccgccgggcccaccgtgacagtgatccccgtgggaagggaggggggcgacattaccattagcggaaaagggttctccacaaccgggttcggcgtctacgtggccgtggctcctgcctctgtgccagagttctatggcaatagcgacaagttctatgggtacgatccatctaaggacaccaccgaaagtcccagcaccatctgggtgtatacaccatcccagaaagcaatcggcagcagattcgcccaaggcaggcccatgaataatgatggctctttcaccattacaatgaaggcccctcctttcgagcagggaaaggatttcgtggtgctgaccaccaaagcacatggcgttggaaagaccgaccacagcgacgatactcggacccctgtgacttacagagaggccacccctgctccaaccggccctaaaactcccatctgataa

SEQ ID NO: 190 - RNA, 876 nt, synthetic construct
  source 1..876: mol_type = other RNA
gtggctcaggccgctattgctacagatggaaagggcatcatcgataaggactcccgcgatgccgtgatcaatgatgccaagctgagggctgctattgctggcgccctggtgaaggctggcttcagctccgccgatgcagtggcactggctcctcggatcgctaaggagatggccaaggagggcgtgctcctgatcaaccaccacaaactgaaggccctgattggggcccagctgggcctgctcacagacgccaagatccagcgggccgctgcagccgtggacctgggcatcaaggccactctggctgctacaatcatcccaaacgcactcgggtccgccgccttcaagaatgccgtgatcgccaacctggttgcagccggaatcgacaaacatctggccagagccacagccgtggccatcgtggctaccgctctgaacccagctctggggcctatcgccaagtttgaactgatcaaggctgagatcgccgcacaggccgccctgctgatcagaagaggagtgcacctgcagaaagccgctatcgagcacgtgatcggacgcgcctttgacgcagccgtggctactgccatcatcagctctccaattctgagcgcaaggatcgtgacacacctggtgagggccggcatcgacaagagcatcgcaatttccctggcccctcacatcgtgaagcggctggctaaggagccactgctcgcctttaataccgcaaaactgatgaaggctattacacgccagatcgtggatgtgatcaccgctgataaggcaatcaagaccgccgaacagctggaaaaggaactgcctgccctggatgacctggtgaaaaaggctatgggcccccctaagcctacctgataa

SEQ ID NO: 191 - RNA, 1308 nt, synthetic construct
  source 1..1308: mol_type = other RNA
tccagcaaccggcctagaagcgtggcccaggccgccatcgccaccgacggcaagggcatcatcgacaaggattcccgggacgccgtgatcaatgacgccaagctgagagccgccatcgccggcgccctggtgaaggccggcttcagcagcgccgacgccgtggccctggcccccagaatcgccaaggagatggccaaggaaggcgtgctgctgatcaatcaccacaagctgaaggccctgatcggcgcccagctgggcctgctgacagatgccaagatccagagagccgctgccgccgtggacctgggcatcaaggccaccctggccgccaccatcatccctaacgccctgggcagcgctgccttcaagaatgccgtgatcgccaacctggtggccgccggcatcgacaagcacctggccagagccacagccgtggccatcgtggccaccgccctgaatcctgccctgggccccatcgccaagttcgagctgatcaaggccgagatcgccgcccaggccgccctgctgatccggcggggcgtgcacctgcagaaggccgccatcgaacacgtgatcggcagagccttcgatgccgccgtggccaccgccatcatcagctcccccatcctgagcgccagaatcgtgacccacctggtgcgggccggcatcgacaagtccatcgccattagcctggcccctcacatcgtgaagcgcctggccaaggagcccctgctggccttcaataccgccaagctgatgaagaatatcaccagacagatcgtggatgtgatcaccgccgataaggccatcaagaccgccgagcagctggagaaagagctgcccgccctggacgacctggtgaaaaaggccatgggcccccctaagcctacaggcggcggcggaggcgctggacctaccgtgaccgtgatccccgtgggcagagagggcggcgatatcaccatcagcggcaagggcttcagcaccaccggcttcggcgtgtacgtggccgtggcccctgccagcgtgcccgagttctacggcaatagcgacaagttctacggctacgaccccagcaaggacaccacagaatctcccagcaccatctgggtgtacacccccagccagaaggccattggcagccggttcgcccagggccggcctatgaacaatgatggcagcttcacaatcaccatgaaggccccacccttcgagcagggcaaggacttcgtggtgctgaccaccaaggcccacggagtgggcaagggcgaccacggcgacgacacacggacccccgtgggctacagatga

SEQ ID NO: 192 - RNA, 1320 nt, synthetic construct
  source 1..1320: mol_type = other RNA
agcagcaaccgcccaaggtctgtcgcacaggctgctatcgccacagatggcaagggaatcattgataaggacagcagggatgctgtgatcaacgacgcaaaactgagagccgccatcgccggcgcactggtgaaagccggcttttcttccgccgacgcagtggctctggctcccagaatcgctaaggaaatggccaaggagggagtgctgctgatcaatcaccacaaactgaaggctctgatcggggctcagctgggcctgctgactgacgctaagattcagagagcagcagcagctgtcgacctgggaatcaaggcaaccctggcagccactatcattccaaatgctctgggctctgccgccttcaaaaacgccgtgattgccaatctcgtggcagctggaatcgataagcacctggcacgggccaccgccgtggctatcgtggccaccgctctgaatcccgccctgggccctattgccaagtttgaactgatcaaagccgagatcgctgcacaggccgccctgctcatccgcaggggggtccacctgcagaaagcagccatcgagcacgtgatcggcagggcctttgatgcagccgtcgccactgccatcatcagctctccaatcctgtctgcccggattgtgactcacctggtgcgcgccggcatcgataagtccatcgcaatctctctggccccacacatcgtgaagagactggccaaagaaccactcctggctttcaataccgccaaactcatgaaaaacatcacccggcagatcgtcgatgtgattaccgccgacaaggctatcaagaccgccgagcagctcgaaaaggagctgcctgccctggacgacctggtgaagaaagcctcctctccacccaaacccaccccaactccaactccaacccctacagctggccccaccgtgaccgtcatcccagtgggaagggaaggaggagacatcacaatctctggaaaggggtttagcacaaccggatttggagtgtacgtcgccgtggcccccgcatccgtgccagaattctacggcaattccgataaattctacgggtacgacccctccaaggacactacagagagcccctctacaatttgggtgtacaccccaagccagaaggccatcggaagcagatttgctcagggccggccaatgaacaacgacggatccttcacaatcacaatgaaagcacctccctttgagcagggcaaggattttgtggtgctgaccaccaaggctcacggggtcggaaagaccgatcacagcgacgatacaagaacccccgtgacataccggtgataa

SEQ ID NO: 193 - RNA, 90 nt, synthetic construct
  source 1..90: mol_type = other RNA
tccggcagcatcctggccatctactccaccgtggcctccagcctggtgctgctggtgagcctgggcgctatctccttcgggtctgggtaa

SEQ ID NO: 194 - RNA, 87 nt, synthetic construct
  source 1..87: mol_type = other RNA
agcgggagcattctggccatctactctaccgtggcctctagcctggtgctgctggtgtctctgggagccattagcttcggatctggc

SEQ ID NO: 195 - RNA, 87 nt, synthetic construct
  source 1..87: mol_type = other RNA
agcggctccatcctggccatctactctaccgtggccagcagcctggtgctgctggtgtccctgggagccatctccttcggctccggg

SEQ ID NO: 196 - RNA, 81 nt, synthetic construct
  source 1..81: mol_type = other RNA
attctggcaatctatagcaccgtggctagcagcctggtgctgctggtgtctctgggagccatcagcttcggctctggctaa

SEQ ID NO: 197 - RNA, 90 nt, synthetic construct
  source 1..90: mol_type = other RNA
agcggcagcattctggcaatctatagcaccgtggctagcagcctggtgctgctggtgtctctgggagccatcagcttcggctctggctaa

SEQ ID NO: 198 - RNA, 90 nt, synthetic construct; identical to SEQ ID NO: 197
  source 1..90: mol_type = other RNA

SEQ ID NO: 199 - RNA, 81 nt, synthetic construct; identical to SEQ ID NO: 196
  source 1..81: mol_type = other RNA

SEQ ID NO: 200 - RNA, 90 nt, synthetic construct
  source 1..90: mol_type = other RNA
agcggctctattctggccatctacagcacagtggccagctctctggtgctgctggttagcctgggcgccatcagcttcggcagcggctaa

SEQ ID NO: 201 - RNA, 90 nt, synthetic construct; identical to SEQ ID NO: 197
  source 1..90: mol_type = other RNA

SEQ ID NO: 202 - protein, 267 aa, Cutibacterium acnes
MKKTHLVAPLLVGAMLVPAALSAPSAHAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 203 - protein, 239 aa, Cutibacterium acnes; identical to SEQ ID NO: 44

SEQ ID NO: 204 - protein, 377 aa, Cutibacterium acnes
SSNRPRSVAQAAIATDGKGIIDKDCRDAVINDAKLRAAIAGALVKAGFSSADAVALAPRIAKEMAKEGVLLINHHKLKALIGAQLGLLTDAKIQRAAAAVDLGIKATLAATIIPNALHSAAFKDAVVANLVAAGVDKKLAKATAVAIAATALNPALGPIAKTEAIKAEIAAQAALLVGRGVHLKKAAIEHIIGRSFDAAVATAIVSSPILNARIVTHLVRAGIDKSLAVQIAPRIIDRLAKEPLLALNTAKLMKNITRQIVDVITADKAIKTAEQLEKELPALDDLVKKACSCPKPTPTPTPTPTPTPKPTPTPTPKPTPTPKPKPTPAPAPTSGATSDESTSRSGGHSQGGSGTHYIHHGVAPVLTHSSDLPSTGF

SEQ ID NO: 205 - protein, 392 aa, Cutibacterium acnes
ASNGNSSITQSAAFSPRATTKISEDCRKAIINDLKLRGAIVGALVKAGLSAADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACTPEPTPTPTPTPTPTPTPAPTPTPAPTPTPAPTPAPTPTPAPTPTPTPTPTPTPTPTHGATTTTPISRTTDRHNLGSHHTRIAAPALIHAKALPATGTGA

SEQ ID NO: 206 - protein, 436 aa, Cutibacterium acnes
AGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVTGAEGPSSDEIDLGIVGGLALTAVVSTVVVCRRYAARI

SEQ ID NO: 207 - protein, 268 aa, synthetic construct
VEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAASGSILAIYSTVASSLVLLVSLGAISFGSG

SEQ ID NO: 208 - protein, 23 aa, Influenza virus
ILAIYSTVASSLVLLVSLGAISF

SEQ ID NO: 209 - protein, 23 aa, Influenza virus
ILAIYSTVASSLVLVVSLGAISF

SEQ ID NO: 210 - protein, 17 aa, Influenza A virus
MKAKLLVLLCTFTATYA

SEQ ID NO: 211 - protein, 17 aa, Influenza A virus
MKAILVVLLYTFATANA

SEQ ID NO: 212 - protein, 16 aa, Influenza A virus
MKTIIALSYILCLVFA

SEQ ID NO: 213 - protein, 15 aa, Influenza B virus
MKAIIVLLMVVTSNA

SEQ ID NO: 214 - protein, 12 aa, SARS CoV-2 virus
MFVFLVLLPLVS

SEQ ID NO: 215 - protein, 21 aa, SARS CoV-2 virus
MFLLTTKRTMFVFLVLLPLVS

SEQ ID NO: 216 - protein, 71 aa, Varicella-zoster virus
MSPCGYYSKWRNRDRPEYRRNLRFRRFFSSIHPNAAAGSGFNGPGVFITSVTGVWLCFLCIFSMFVTAVVS

SEQ ID NO: 217 - protein, 30 aa, Varicella-zoster virus
MGTVNKPVVGVLMGFGIITGTLRITNPVRA

SEQ ID NO: 218 - protein, 20 aa, Varicella-zoster virus
MFLIQCLISAVIFYIQVTNA

SEQ ID NO: 219 - protein, 21 aa, Varicella-zoster virus
MQALGIKTEHFIIMCLLSGHA

SEQ ID NO: 220 - protein, 23 aa, Measles virus
MGLKVNVSAIFMAVLLTLQTPTG

SEQ ID NO: 221 - protein, 21 aa, Rubella virus
MGAAAALTAVVLQGYNPPAYG

SEQ ID NO: 222 - protein, 23 aa, Rubella virus
MGAPQAFLAGLLLAAVAVGTARA

SEQ ID NO: 223 - protein, 19 aa, Mumps virus
MKVFLVTCLGFAVFSSSVC

SEQ ID NO: 224 - protein, 32 aa, Ebola virus
MGVTGILQLPRDRFKRTSFFLWVIILFQRTFS

SEQ ID NO: 225 - protein, 16 aa, Smallpox virus
MRSLIIFLLFPSIIYS

SEQ ID NO: 226 - protein, 19 aa, Rabies virus
MVPQALLFVPLLVFPLCFG

SEQ ID NO: 227 - protein, 17 aa, Influenza A virus
MKAILVVLLYTFTTANA

SEQ ID NO: 228 - protein, 17 aa, Influenza A virus
MKVKLLVLLCTFTATYA

SEQ ID NO: 229 - protein, 17 aa, Influenza A virus
MKAILVVMLYTFTTANA

SEQ ID NO: 230 - protein, 16 aa, Influenza A virus
MKTIIALSYILCLVFT

SEQ ID NO: 231 - protein, 16 aa, synthetic construct
MKTIIALSNILCLVFA

SEQ ID NO: 232 - protein, 16 aa, synthetic construct
MKAIIALSNILCLVFA

SEQ ID NO: 233 - protein, 16 aa, Influenza A virus
MKTIIALSCILCLVFA

SEQ ID NO: 234 - protein, 16 aa, Influenza A virus
MKTIIALSHILCLVFA

SEQ ID NO: 235 - protein, 15 aa, Influenza B virus
MKAIIVLLMVVTSSA

SEQ ID NO: 236 - protein, 15 aa, Influenza B virus
MEAIIVLLMVVTSNA

SEQ ID NO: 237 - protein, 17 aa, Influenza B virus
  VARIANT 3: note = A or V
  VARIANT 4: note = I or K
  VARIANT 6: note = V or L
  VARIANT 8: note = L or M
  VARIANT 10: note = Y or C
  VARIANT 13: note = T or A
  VARIANT 16: note = N or Y
  VARIANT 14: note = T or A
  VARIANT 15: note = T or A
MKXXLXVXLXTFXXXXA

SEQ ID NO: 238 - protein, 16 aa, Influenza B virus
  VARIANT 3: note = T or A
  VARIANT 9: note = Y, N, C or H
  VARIANT 16: note = T or A
MKXIIALSXILCLVFX

SEQ ID NO: 239 - protein, 15 aa, Influenza B virus
  VARIANT 14: note = S or N
MKAIIVLLMVVTSXA

SEQ ID NO: 240 - protein, 15 aa, Influenza B virus
  VARIANT 2: note = K or E
MXAIIVLLMVVTSNA

SEQ ID NO: 241 - protein, 21 aa, Influenza virus
ILWISFAISCFLLCVVLLGFI

SEQ ID NO: 242 - protein, 21 aa, Influenza virus
STAASSLAVTLMLAIFIVYMV

SEQ ID NO: 243 - protein, 21 aa, SARS CoV-2 virus
WYIWLGFIAGLIAIVMVTIML

SEQ ID NO: 244 - protein, 21 aa, Varicella-zoster virus
FGALAVGLLVLAGLVAAFFAY

SEQ ID NO: 245 - protein, 21 aa, Varicella-zoster virus
AAWTGGLAAVVLLCLVIFLIC

SEQ ID NO: 246 - protein, 21 aa, Varicella-zoster virus
IIIPIVASVMILTAMVIVIVI

SEQ ID NO: 247 - protein, 21 aa, Varicella-zoster virus
YFWCVQLKMIFFAWFVYGMYL

SEQ ID NO: 248 - protein, 21 aa, Measles virus
IVYILIAVCLGGLIGIPALIC

SEQ ID NO: 249 - protein, 21 aa, Rubella virus
LDHAFAAFVLLVPWVLIFMVC

SEQ ID NO: 250 - protein, 21 aa, Rubella virus
WWQLTLGAICALLLAGLLACC

SEQ ID NO: 251 - protein, 21 aa, Mumps virus
IVAALVLSILSIIISLLFCCW

SEQ ID NO: 252 - protein, 21 aa, Ebola virus
WIPAGIGVTGVIIAVIALFCI

SEQ ID NO: 253 - protein, 21 aa, Rabies virus
VLLSAGALTALMLIIFLMTCW

SEQ ID NO: 254 - protein, 5 aa, synthetic construct
DGGGS

SEQ ID NO: 255 - protein, 5 aa, synthetic construct
TGEKP

SEQ ID NO: 256 - protein, 4 aa, synthetic construct
GGRR

SEQ ID NO: 257 - protein, 5 aa, synthetic construct
  REPEAT 1..5: note = 1, 2, 3, 4 or 5 repeats
GGGGS

SEQ ID NO: 258 - protein, 14 aa, synthetic construct
EGKSSGSGSESKVD

SEQ ID NO: 259 - protein, 18 aa, synthetic construct
KESGSVSSEQLAQFRSLD

SEQ ID NO: 260 - protein, 8 aa, synthetic construct
GGRRGGGS

SEQ ID NO: 261 - protein, 9 aa, synthetic construct
LRQRDGERP

SEQ ID NO: 262 - protein, 12 aa, synthetic construct
LRQKDGGGSERP

SEQ ID NO: 263 - protein, 18 aa, synthetic construct
GSTSGSGKPGSGEGSTKG

SEQ ID NO: 264 - RNA, 11 nt, synthetic construct
  source 1..11: mol_type = other RNA
gggatcctacc

SEQ ID NO: 265 - RNA, 140 nt, synthetic construct
  source 1..140: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 266 - RNA, 100 nt, synthetic construct
  source 1..100: mol_type = other RNA
cgggtggcatccctgtgacccctccccagtgcctctcctggccctggaagttgccactccagtgcccaccagccttgtcctaataaaattaagttgcatc

SEQ ID NO: 267 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 268 - protein, 256 aa, synthetic construct
MKAILVVLLYTFATANAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 269 - protein, 254 aa, synthetic construct
MKAIIVLLMVVTSNAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 270 - protein, 258 aa, synthetic construct
MVPQALLFVPLLVFPLCFGVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 271 - protein, 259 aa, synthetic construct
MFLIQCLISAVIFYIQVTNAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 272 - protein, 271 aa, synthetic construct
MGVTGILQLPRDRFKRTSFFLWVIILFQRTFSVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 273 - protein, 285 aa, synthetic construct
MKAILVVLLYTFATANAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAASGSILAIYSTVASSLVLVVSLGAISFGSG

SEQ ID NO: 274 - protein, 281 aa, synthetic construct
MKAIIVLLMVVTSNAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAASGSSTAASSLAVTLMLAIFIVYMVGSG

SEQ ID NO: 275 - protein, 285 aa, synthetic construct
MVPQALLFVPLLVFPLCFGVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAASGSVLLSAGALTALMLIIFLMTCWGSG

SEQ ID NO: 276 - protein, 286 aa, synthetic construct
MFLIQCLISAVIFYIQVTNAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAASGSIIIPIVASVMILTAMVIVIVIGSG

SEQ ID NO: 277 - protein, 298 aa, synthetic construct
MGVTGILQLPRDRFKRTSFFLWVIILFQRTFSVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAASGSWIPAGIGVTGVIIAVIALFCIGSG

SEQ ID NO: 278 - protein, 400 aa, synthetic construct
MAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 279 - protein, 416 aa, synthetic construct
MKAILVVLLYTFATANAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 280 - protein, 414 aa, synthetic construct
MKAIIVLLMVVTSNAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 281 - protein, 418 aa, synthetic construct
MVPQALLFVPLLVFPLCFGAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 282 - protein, 419 aa, synthetic construct
MFLIQCLISAVIFYIQVTNAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 283 - protein, 431 aa, synthetic construct
MGVTGILQLPRDRFKRTSFFLWVIILFQRTFSAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 284 - protein, 445 aa, synthetic construct
MKAILVVLLYTFATANAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVTSGSILAIYSTVASSLVLVVSLGAISFGSG

SEQ ID NO: 285 - protein, 441 aa, synthetic construct
MKAIIVLLMVVTSNAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVTSGSSTAASSLAVTLMLAIFIVYMVGSG

SEQ ID NO: 286 - protein, 445 aa, synthetic construct
MVPQALLFVPLLVFPLCFGAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVTSGSVLLSAGALTALMLIIFLMTCWGSG

SEQ ID NO: 287 - protein, 446 aa, synthetic construct
MFLIQCLISAVIFYIQVTNAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVTSGSIIIPIVASVMILTAMVIVIVIGSG

SEQ ID NO: 288 - protein, 458 aa, synthetic construct
MGVTGILQLPRDRFKRTSFFLWVIILFQRTFSAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVTSGSWIPAGIGVTGVIIAVIALFCIGSG

SEQ ID NO: 289 - RNA, 723 nt, synthetic construct
  source 1..723: mol_type = other RNA
atggtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacaccaaacgatgtggccactgtgtacgtgagaaccaacttctccaagaccatctggcaggtcagagccaaccgcgatcgctacatcctgggccacaagtccgccgccgtgtataagaccctgaaccatgccatcaccaaggccgtgggcgtcagactgaaccccaagaccaccgtgggcaacatccaggccgctagaacagagctgctggccgcctaccagaccgccttcaattcacccgacgtgaagaaggccgcttaa

SEQ ID NO: 290 - RNA, 771 nt, synthetic construct
  source 1..771: mol_type = other RNA
atgaaggccattctggtggtgctgctgtacacatttgctacagccaacgccgtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacaccaaacgatgtggccactgtgtacgtgagaaccaacttctccaagaccatctggcaggtcagagccaaccgcgatcgctacatcctgggccacaagtccgccgccgtgtataagaccctgaaccatgccatcaccaaggccgtgggcgtcagactgaaccccaagaccaccgtgggcaacatccaggccgctagaacagagctgctggccgcctaccagaccgccttcaattcacccgacgtgaagaaggccgcttaa

SEQ ID NO: 291 - RNA, 765 nt, synthetic construct
  source 1..765: mol_type = other RNA
atgaaggctatcatcgtgctgctgatggtggtgacctccaacgccgtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacaccaaacgatgtggccactgtgtacgtgagaaccaacttctccaagaccatctggcaggtcagagccaaccgcgatcgctacatcctgggccacaagtccgccgccgtgtataagaccctgaaccatgccatcaccaaggccgtgggcgtcagactgaaccccaagaccaccgtgggcaacatccaggccgctagaacagagctgctggccgcctaccagaccgccttcaattcacccgacgtgaagaaggccgcttaa

SEQ ID NO: 292 - RNA, 777 nt, synthetic construct
  source 1..777: mol_type = other RNA
atggtgccccaggcactgctgttcgtgccactgctggtgtttccactgtgtttcggcgtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacaccaaacgatgtggccactgtgtacgtgagaaccaacttctccaagaccatctggcaggtcagagccaaccgcgatcgctacatcctgggccacaagtccgccgccgtgtataagaccctgaaccatgccatcaccaaggccgtgggcgtcagactgaaccccaagaccaccgtgggcaacatccaggccgctagaacagagctgctggccgcctaccagaccgccttcaattcacccgacgtgaagaaggccgcttaa

SEQ ID NO: 293 - RNA, 780 nt, synthetic construct
  source 1..780: mol_type = other RNA
atgttcctgatccagtgtctgatctccgccgtgatcttctatatccaggtgaccaacgcagtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacaccaaacgatgtggccactgtgtacgtgagaaccaacttctccaagaccatctggcaggtcagagccaaccgcgatcgctacatcctgggccacaagtccgccgccgtgtataagaccctgaaccatgccatcaccaaggccgtgggcgtcagactgaaccccaagaccaccgtgggcaacatccaggccgctagaacagagctgctggccgcctaccagaccgccttcaattcacccgacgtgaagaaggccgcttaa

SEQ ID NO: 294 - RNA, 816 nt, synthetic construct
  source 1..816: mol_type = other RNA
atgggagtgaccggaatcctgcagctgcccagagataggttcaagcggaccagcttcttcctgtgggtcattatcctgtttcagagaacattctctgtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacaccaaacgatgtggccactgtgtacgtgagaaccaacttctccaagaccatctggcaggtcagagccaaccgcgatcgctacatcctgggccacaagtccgccgccgtgtataagaccctgaaccatgccatcaccaaggccgtgggcgtcagactgaaccccaagaccaccgtgggcaacatccaggccgctagaacagagctgctggccgcctaccagaccgccttcaattcacccgacgtgaagaaggccgcttaa

SEQ ID NO: 295 - RNA, 858 nt, synthetic construct
  source 1..858: mol_type = other RNA
atgaaggccattctggtggtgctgctgtacacatttgctacagccaacgccgtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacaccaaacgatgtggccactgtgtacgtgagaaccaacttctccaagaccatctggcaggtcagagccaaccgcgatcgctacatcctgggccacaagtccgccgccgtgtataagaccctgaaccatgccatcaccaaggccgtgggcgtcagactgaaccccaagaccaccgtgggcaacatccaggccgctagaacagagctgctggccgcctaccagaccgccttcaattcacccgacgtgaagaaggccgcttccggcagcatcctggccatctacagcaccgtggctagctccctggtgctggtggtgagcctgggagctattagctttggaagcggataa

SEQ ID NO: 296 - RNA, 846 nt, synthetic construct
  source 1..846: mol_type = other RNA
atgaaggctatcatcgtgctgctgatggtggtgacctccaacgccgtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacaccaaacgatgtggccactgtgtacgtgagaaccaacttctccaagaccatctggcaggtcagagccaaccgcgatcgctacatcctgggccacaagtccgccgccgtgtataagaccctgaaccatgccatcaccaaggccgtgggcgtcagactgaaccccaagaccaccgtgggcaacatccaggccgctagaacagagctgctggccgcctaccagaccgccttcaattcacccgacgtgaagaaggccgctagcggcagctctacagccgcaagcagcctggctgtgacactgatgctggccattttcattgtgtacatggtgggctccggataa

SEQ ID NO: 297 - RNA, 858 nt, synthetic construct
  source 1..858: mol_type = other RNA
atggtgccccaggcactgctgttcgtgccactgctggtgtttccactgtgtttcggcgtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacaccaaacgatgtggccactgtgtacgtgagaaccaacttctccaagaccatctggcaggtcagagccaaccgcgatcgctacatcctgggccacaagtccgccgccgtgtataagaccctgaaccatgccatcaccaaggccgtgggcgtcagactgaaccccaagaccaccgtgggcaacatccaggccgctagaacagagctgctggccgcctaccagaccgccttcaattcacccgacgtgaagaaggccgctagcggatccgtgctgctgtctgccggagctctgacagcactgatgctgatcattttcctgatgacctgctggggcagcggctaa

SEQ ID NO: 298 - RNA, 861 nt, synthetic construct
  source 1..861: mol_type = other RNA
atgttcctgatccagtgtctgatctccgccgtgatcttctatatccaggtgaccaacgcagtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacaccaaacgatgtggccactgtgtacgtgagaaccaacttctccaagaccatctggcaggtcagagccaaccgcgatcgctacatcctgggccacaagtccgccgccgtgtataagaccctgaaccatgccatcaccaaggccgtgggcgtcagactgaaccccaagaccaccgtgggcaacatccaggccgctagaacagagctgctggccgcctaccagaccgccttcaattcacccgacgtgaagaaggccgctagcggaagcattattatccccatcgtggcctctgtgatgatcctgaccgcaatggtcattgtgatcgtgatcggcagcggctaa

SEQ ID NO: 299 - RNA, 897 nt, synthetic construct
  source 1..897: mol_type = other RNA
atgggagtgaccggaatcctgcagctgcccagagataggttcaagcggaccagcttcttcctgtgggtcattatcctgtttcagagaacattctctgtcgagcccaccaccaccatctccgccacatccacacacgaactgtccgcaagcgacgccagaaacagcatccagctgctgaacgcccacatcgccaccctccagagcgtgcagaagagtgtgcccggcagcgattatagcgaccagatccgggacctgctcaaggccgcattcgacctgcgcggcctgatcgagacactggctcatggcgggatccccttctatgatcccagcaccatcatgcccaggatcaagctggtcgccactaccatcgacaccatccacaccgccaccaccaccctgcagaacaaagtgcgccccgcccacgtggagctggggctggaggtgaccaaagcggtgctgctgacagccaaccctgcatccaccgccaaggagctggacgcagagggcgctgcactgaaggcccggctcgagaaggtgtcccagtatcccgacctgacaccaaacgatgtggccactgtgtacgtgagaaccaacttctccaagaccatctggcaggtcagagccaaccgcgatcgctacatcctgggccacaagtccgccgccgtgtataagaccctgaaccatgccatcaccaaggccgtgggcgtcagactgaaccccaagaccaccgtgggcaacatccaggccgctagaacagagctgctggccgcctaccagaccgccttcaattcacccgacgtgaagaaggccgcttctggatcctggatccctgccggaatcggggtgaccggcgtgatcatcgccgtgatcgccctgttctgtattggcagcggataa

SEQ ID NO: 300 - RNA, 1203 nt, synthetic construct
  source 1..1203: mol_type = other RNA
atggctgggccaacagtgacagtgatccccgtgggacgcgagggcggagacattaccatctccgggaagggctttagcacaacagggttcggtgtgtatgtggctgtggccccagcaagcgtgccagagttctacgggaactccgataaattttatgggtatgaccccagtaaagacacaaccgagagccccagcacaatttgggtttacaccccctctcagaaagctattggctcccggttcgcccagggcagaccaatgaataatgacgggagctttaccatcactatgaaggcccccccattcgagcagggaaaggactttgtggtcctgactaccaaggctcacggagtgggcaaaaccgaccactctgacgacactcggacaccggtgacataccgggaggctactcccgctccaacaggccctaaaactccaatcgcccccagcaagcagccttcaaagcaggctgcccccagcaagcaggtgaagcctagcaagcaggctggccccaacaagcagagcactactcctcagcagaagaccgcagagcacagatctcagaccccagcagctcatcgcactatgactaagcaggtgtgtaccatcggagcctcaaaggtgaccagcggatccctgacatggggaatccggactagctttacttcctacctgcgcgggcccatcgcaaacggcagctggaagctgtctgggggggccaattggaacggcagcgccttcacattcccactgacttctggatccttcgaccccgccaccaaatccggctcactgaagtacagcgggagcgtgcacatgactggacaccatggaattctggacatgacactcgctgaaccctctctgcagatcaagggctctactgggcacctgtatctggatgtgaagtcctcttctatggacggcaagaagacaaactatgggagagtggattttgccaccttcggagtgagcgtgagcggaaatgctgcaatcaaagggtcccccgtgaaactgaccgctaccggagcaaaagcctttgccggcttttacagagcaggagagcccatgaaccccctgagcaccaatctgacactgagcgctgagaaagtttgtcacaatgtgaccgttgatgcagttaccgggaaagtgatcggcgacgattccggaaagggggcaggaaggggactgcctgtcacttga

SEQ ID NO: 301 - RNA, 1251 nt, synthetic construct
  source 1..1251: mol_type = other RNA
atgaaggccattctggtggtgctgctgtacacatttgctacagccaacgccgctgggccaacagtgacagtgatccccgtgggacgcgagggcggagacattaccatctccgggaagggctttagcacaacagggttcggtgtgtatgtggctgtggccccagcaagcgtgccagagttctacgggaactccgataaattttatgggtatgaccccagtaaagacacaaccgagagccccagcacaatttgggtttacaccccctctcagaaagctattggctcccggttcgcccagggcagaccaatgaataatgacgggagctttaccatcactatgaaggcccccccattcgagcagggaaaggactttgtggtcctgactaccaaggctcacggagtgggcaaaaccgaccactctgacgacactcggacaccggtgacataccgggaggctactcccgctccaacaggccctaaaactccaatcgcccccagcaagcagccttcaaagcaggctgcccccagcaagcaggtgaagcctagcaagcaggctggccccaacaagcagagcactactcctcagcagaagaccgcagagcacagatctcagaccccagcagctcatcgcactatgactaagcaggtgtgtaccatcggagcctcaaaggtgaccagcggatccctgacatggggaatccggactagctttacttcctacctgcgcgggcccatcgcaaacggcagctggaagctgtctgggggggccaattggaacggcagcgccttcacattcccactgacttctggatccttcgaccccgccaccaaatccggctcactgaagtacagcgggagcgtgcacatgactggacaccatggaattctggacatgacactcgctgaaccctctctgcagatcaagggctctactgggcacctgtatctggatgtgaagtcctcttctatggacggcaagaagacaaactatgggagagtggattttgccaccttcggagtgagcgtgagcggaaatgctgcaatcaaagggtcccccgtgaaactgaccgctaccggagcaaaagcctttgccggcttttacagagcaggagagcccatgaaccccctgagcaccaatctgacactgagcgctgagaaagtttgtcacaatgtgaccgttgatgcagttaccgggaaagtgatcggcgacgattccggaaagggggcaggaaggggactgcctgtcacttga

SEQ ID NO: 302 - RNA, 1245 nt, synthetic construct
  source 1..1245: mol_type = other RNA
atgaaggctatcatcgtgctgctgatggtggtgacctccaacgccgctgggccaacagtgacagtgatccccgtgggacgcgagggcggagacattaccatctccgggaagggctttagcacaacagggttcggtgtgtatgtggctgtggccccagcaagcgtgccagagttctacgggaactccgataaattttatgggtatgaccccagtaaagacacaaccgagagccccagcacaatttgggtttacaccccctctcagaaagctattggctcccggttcgcccagggcagaccaatgaataatgacgggagctttaccatcactatgaaggcccccccattcgagcagggaaaggactttgtggtcctgactaccaaggctcacggagtgggcaaaaccgaccactctgacgacactcggacaccggtgacataccgggaggctactcccgctccaacaggccctaaaactccaatcgcccccagcaagcagccttcaaagcaggctgcccccagcaagcaggtgaagcctagcaagcaggctggccccaacaagcagagcactactcctcagcagaagaccgcagagcacagatctcagaccccagcagctcatcgcactatgactaagcaggtgtgtaccatcggagcctcaaaggtgaccagcggatccctgacatggggaatccggactagctttacttcctacctgcgcgggcccatcgcaaacggcagctggaagctgtctgggggggccaattggaacggcagcgccttcacattcccactgacttctggatccttcgaccccgccaccaaatccggctcactgaagtacagcgggagcgtgcacatgactggacaccatggaattctggacatgacactcgctgaaccctctctgcagatcaagggctctactgggcacctgtatctggatgtgaagtcctcttctatggacggcaagaagacaaactatgggagagtggattttgccaccttcggagtgagcgtgagcggaaatgctgcaatcaaagggtcccccgtgaaactgaccgctaccggagcaaaagcctttgccggcttttacagagcaggagagcccatgaaccccctgagcaccaatctgacactgagcgctgagaaagtttgtcacaatgtgaccgttgatgcagttaccgggaaagtgatcggcgacgattccggaaagggggcaggaaggggactgcctgtcacttga

SEQ ID NO: 303 - RNA, 1257 nt, synthetic construct
  source 1..1257: mol_type = other RNA
atggtgccccaggcactgctgttcgtgccactgctggtgtttccactgtgtttcggcgctgggccaacagtgacagtgatccccgtgggacgcgagggcggagacattaccatctccgggaagggctttagcacaacagggttcggtgtgtatgtggctgtggccccagcaagcgtgccagagttctacgggaactccgataaattttatgggtatgaccccagtaaagacacaaccgagagccccagcacaatttgggtttacaccccctctcagaaagctattggctcccggttcgcccagggcagaccaatgaataatgacgggagctttaccatcactatgaaggcccccccattcgagcagggaaaggactttgtggtcctgactaccaaggctcacggagtgggcaaaaccgaccactctgacgacactcggacaccggtgacataccgggaggctactcccgctccaacaggccctaaaactccaatcgcccccagcaagcagccttcaaagcaggctgcccccagcaagcaggtgaagcctagcaagcaggctggccccaacaagcagagcactactcctcagcagaagaccgcagagcacagatctcagaccccagcagctcatcgcactatgactaagcaggtgtgtaccatcggagcctcaaaggtgaccagcggatccctgacatggggaatccggactagctttacttcctacctgcgcgggcccatcgcaaacggcagctggaagctgtctgggggggccaattggaacggcagcgccttcacattcccactgacttctggatccttcgaccccgccaccaaatccggctcactgaagtacagcgggagcgtgcacatgactggacaccatggaattctggacatgacactcgctgaaccctctctgcagatcaagggctctactgggcacctgtatctggatgtgaagtcctcttctatggacggcaagaagacaaactatgggagagtggattttgccaccttcggagtgagcgtgagcggaaatgctgcaatcaaagggtcccccgtgaaactgaccgctaccggagcaaaagcctttgccggcttttacagagcaggagagcccatgaaccccctgagcaccaatctgacactgagcgctgagaaagtttgtcacaatgtgaccgttgatgcagttaccgggaaagtgatcggcgacgattccggaaagggggcaggaaggggactgcctgtcacttga

SEQ ID NO: 304 - RNA, 1260 nt, synthetic construct
  source 1..1260: mol_type = other RNA
atgttcctgatccagtgtctgatctccgccgtgatcttctatatccaggtgaccaacgcagctgggccaacagtgacagtgatccccgtgggacgcgagggcggagacattaccatctccgggaagggctttagcacaacagggttcggtgtgtatgtggctgtggccccagcaagcgtgccagagttctacgggaactccgataaattttatgggtatgaccccagtaaagacacaaccgagagccccagcacaatttgggtttacaccccctctcagaaagctattggctcccggttcgcccagggcagaccaatgaataatgacgggagctttaccatcactatgaaggcccccccattcgagcagggaaaggactttgtggtcctgactaccaaggctcacggagtgggcaaaaccgaccactctgacgacactcggacaccggtgacataccgggaggctactcccgctccaacaggccctaaaactccaatcgcccccagcaagcagccttcaaagcaggctgcccccagcaagcaggtgaagcctagcaagcaggctggccccaacaagcagagcactactcctcagcagaagaccgcagagcacagatctcagaccccagcagctcatcgcactatgactaagcaggtgtgtaccatcggagcctcaaaggtgaccagcggatccctgacatggggaatccggactagctttacttcctacctgcgcgggcccatcgcaaacggcagctggaagctgtctgggggggccaattggaacggcagcgccttcacattcccactgacttctggatccttcgaccccgccaccaaatccggctcactgaagtacagcgggagcgtgcacatgactggacaccatggaattctggacatgacactcgctgaaccctctctgcagatcaagggctctactgggcacctgtatctggatgtgaagtcctcttctatggacggcaagaagacaaactatgggagagtggattttgccaccttcggagtgagcgtgagcggaaatgctgcaatcaaagggtcccccgtgaaactgaccgctaccggagcaaaagcctttgccggcttttacagagcaggagagcccatgaaccccctgagcaccaatctgacactgagcgctgagaaagtttgtcacaatgtgaccgttgatgcagttaccgggaaagtgatcggcgacgattccggaaagggggcaggaaggggactgcctgtcacttga

SEQ ID NO: 305 - RNA, 1296 nt, synthetic construct
  source 1..1296: mol_type = other RNA
atgggagtgaccggaatcctgcagctgcccagagataggttcaagcggaccagcttcttcctgtgggtcattatcctgtttcagagaacattctctgctgggccaacagtgacagtgatccccgtgggacgcgagggcggagacattaccatctccgggaagggctttagcacaacagggttcggtgtgtatgtggctgtggccccagcaagcgtgccagagttctacgggaactccgataaattttatgggtatgaccccagtaaagacacaaccgagagccccagcacaatttgggtttacaccccctctcagaaagctattggctcccggttcgcccagggcagaccaatgaataatgacgggagctttaccatcactatgaaggcccccccattcgagcagggaaaggactttgtggtcctgactaccaaggctcacggagtgggcaaaaccgaccactctgacgacactcggacaccggtgacataccgggaggctactcccgctccaacaggccctaaaactccaatcgcccccagcaagcagccttcaaagcaggctgcccccagcaagcaggtgaagcctagcaagcaggctggccccaacaagcagagcactactcctcagcagaagaccgcagagcacagatctcagaccccagcagctcatcgcactatgactaagcaggtgtgtaccatcggagcctcaaaggtgaccagcggatccctgacatggggaatccggactagctttacttcctacctgcgcgggcccatcgcaaacggcagctggaagctgtctgggggggccaattggaacggcagcgccttcacattcccactgacttctggatccttcgaccccgccaccaaatccggctcactgaagtacagcgggagcgtgcacatgactggacaccatggaattctggacatgacactcgctgaaccctctctgcagatcaagggctctactgggcacctgtatctggatgtgaagtcctcttctatggacggcaagaagacaaactatgggagagtggattttgccaccttcggagtgagcgtgagcggaaatgctgcaatcaaagggtcccccgtgaaactgaccgctaccggagcaaaagcctttgccggcttttacagagcaggagagcccatgaaccccctgagcaccaatctgacactgagcgctgagaaagtttgtcacaatgtgaccgttgatgcagttaccgggaaagtgatcggcgacgattccggaaagggggcaggaaggggactgcctgtcacttga

SEQ ID NO: 306 - RNA, 1338 nt, synthetic construct
  source 1..1338: mol_type = other RNA
atgaaggccattctggtggtgctgctgtacacatttgctacagccaacgccgctgggccaacagtgacagtgatccccgtgggacgcgagggcggagacattaccatctccgggaagggctttagcacaacagggttcggtgtgtatgtggctgtggccccagcaagcgtgccagagttctacgggaactccgataaattttatgggtatgaccccagtaaagacacaaccgagagccccagcacaatttgggtttacaccccctctcagaaagctattggctcccggttcgcccagggcagaccaatgaataatgacgggagctttaccatcactatgaaggcccccccattcgagcagggaaaggactttgtggtcctgactaccaaggctcacggagtgggcaaaaccgaccactctgacgacactcggacaccggtgacataccgggaggctactcccgctccaacaggccctaaaactccaatcgcccccagcaagcagccttcaaagcaggctgcccccagcaagcaggtgaagcctagcaagcaggctggccccaacaagcagagcactactcctcagcagaagaccgcagagcacagatctcagaccccagcagctcatcgcactatgactaagcaggtgtgtaccatcggagcctcaaaggtgaccagcggatccctgacatggggaatccggactagctttacttcctacctgcgcgggcccatcgcaaacggcagctggaagctgtctgggggggccaattggaacggcagcgccttcacattcccactgacttctggatccttcgaccccgccaccaaatccggctcactgaagtacagcgggagcgtgcacatgactggacaccatggaattctggacatgacactcgctgaaccctctctgcagatcaagggctctactgggcacctgtatctggatgtgaagtcctcttctatggacggcaagaagacaaactatgggagagtggattttgccaccttcggagtgagcgtgagcggaaatgctgcaatcaaagggtcccccgtgaaactgaccgctaccggagcaaaagcctttgccggcttttacagagcaggagagcccatgaaccccctgagcaccaatctgacactgagcgctgagaaagtttgtcacaatgtgaccgttgatgcagttaccgggaaagtgatcggcgacgattccggaaagggggcaggaaggggactgcctgtcacttccggcagcatcctggccatctacagcaccgtggctagctccctggtgctggtggtgagcctgggagctattagctttggaagcggatga

SEQ ID NO: 307 - RNA, 1326 nt, synthetic construct
  source 1..1326: mol_type = other RNA
atgaaggctatcatcgtgctgctgatggtggtgacctccaacgccgctgggccaacagtgacagtgatccccgtgggacgcgagggcggagacattaccatctccgggaagggctttagcacaacagggttcggtgtgtatgtggctgtggccccagcaagcgtgccagagttctacgggaactccgataaattttatgggtatgaccccagtaaagacacaaccgagagccccagcacaatttgggtttacaccccctctcagaaagctattggctcccggttcgcccagggcagaccaatgaataatgacgggagctttaccatcactatgaaggcccccccattcgagcagggaaaggactttgtggtcctgactaccaaggctcacggagtgggcaaaaccgaccactctgacgacactcggacaccggtgacataccgggaggctactcccgctccaacaggccctaaaactccaatcgcccccagcaagcagccttcaaagcaggctgcccccagcaagcaggtgaagcctagcaagcaggctggccccaacaagcagagcactactcctcagcagaagaccgcagagcacagatctcagaccccagcagctcatcgcactatgactaagcaggtgtgtaccatcggagcctcaaaggtgaccagcggatccctgacatggggaatccggactagctttacttcctacctgcgcgggcccatcgcaaacggcagctggaagctgtctgggggggccaattggaacggcagcgccttcacattcccactgacttctggatccttcgaccccgccaccaaatccggctcactgaagtacagcgggagcgtgcacatgactggacaccatggaattctggacatgacactcgctgaaccctctctgcagatcaagggctctactgggcacctgtatctggatgtgaagtcctcttctatggacggcaagaagacaaactatgggagagtggattttgccaccttcggagtgagcgtgagcggaaatgctgcaatcaaagggtcccccgtgaaactgaccgctaccggagcaaaagcctttgccggcttttacagagcaggagagcccatgaaccccctgagcaccaatctgacactgagcgctgagaaagtttgtcacaatgtgaccgttgatgcagttaccgggaaagtgatcggcgacgattccggaaagggggcaggaaggggactgcctgtcactagcggcagctctacagccgcaagcagcctggctgtgacactgatgctggccattttcattgtgtacatggtgggctccggatga

SEQ ID NO: 308 - RNA, 1338 nt, synthetic construct
  source 1..1338: mol_type = other RNA
atggtgccccaggcactgctgttcgtgccactgctggtgtttccactgtgtttcggcgctgggccaacagtgacagtgatccccgtgggacgcgagggcggagacattaccatctccgggaagggctttagcacaacagggttcggtgtgtatgtggctgtggccccagcaagcgtgccagagttctacgggaactccgataaattttatgggtatgaccccagtaaagacacaaccgagagccccagcacaatttgggtttacaccccctctcagaaagctattggctcccggttcgcccagggcagaccaatgaataatgacgggagctttaccatcactatgaaggcccccccattcgagcagggaaaggactttgtggtcctgactaccaaggctcacggagtgggcaaaaccgaccactctgacgacactcggacaccggtgacataccgggaggctactcccgctccaacaggccctaaaactccaatcgcccccagcaagcagccttcaaagcaggctgcccccagcaagcaggtgaagcctagcaagcaggctggccccaacaagcagagcactactcctcagcagaagaccgcagagcacagatctcagaccccagcagctcatcgcactatgactaagcaggtgtgtaccatcggagcctcaaaggtgaccagcggatccctgacatggggaatccggactagctttacttcctacctgcgcgggcccatcgcaaacggcagctggaagctgtctgggggggccaattggaacggcagcgccttcacattcccactgacttctggatccttcgaccccgccaccaaatccggctcactgaagtacagcgggagcgtgcacatgactggacaccatggaattctggacatgacactcgctgaaccctctctgcagatcaagggctctactgggcacctgtatctggatgtgaagtcctcttctatggacggcaagaagacaaactatgggagagtggattttgccaccttcggagtgagcgtgagcggaaatgctgcaatcaaagggtcccccgtgaaactgaccgctaccggagcaaaagcctttgccggcttttacagagcaggagagcccatgaaccccctgagcaccaatctgacactgagcgctgagaaagtttgtcacaatgtgaccgttgatgcagttaccgggaaagtgatcggcgacgattccggaaagggggcaggaaggggactgcctgtcactagcggatccgtgctgctgtctgccggagctctgacagcactgatgctgatcattttcctgatgacctgctggggcagcggctga

SEQ ID NO: 309 - RNA, 1341 nt, synthetic construct
  source 1..1341: mol_type = other RNA
atgttcctgatccagtgtctgatctccgccgtgatcttctatatccaggtgaccaacgcagctgggccaacagtgacagtgatccccgtgggacgcgagggcggagacattaccatctccgggaagggctttagcacaacagggttcggtgtgtatgtggctgtggccccagcaagcgtgccagagttctacgggaactccgataaattttatgggtatgaccccagtaaagacacaaccgagagccccagcacaatttgggtttacaccccctctcagaaagctattggctcccggttcgcccagggcagaccaatgaataatgacgggagctttaccatcactatgaaggcccccccattcgagcagggaaaggactttgtggtcctgactaccaaggctcacggagtgggcaaaaccgaccactctgacgacactcggacaccggtgacataccgggaggctactcccgctccaacaggccctaaaactccaatcgcccccagcaagcagccttcaaagcaggctgcccccagcaagcaggtgaagcctagcaagcaggctggccccaacaagcagagcactactcctcagcagaagaccgcagagcacagatctcagaccccagcagctcatcgcactatgactaagcaggtgtgtaccatcggagcctcaaaggtgaccagcggatccctgacatggggaatccggactagctttacttcctacctgcgcgggcccatcgcaaacggcagctggaagctgtctgggggggccaattggaacggcagcgccttcacattcccactgacttctggatccttcgaccccgccaccaaatccggctcactgaagtacagcgggagcgtgcacatgactggacaccatggaattctggacatgacactcgctgaaccctctctgcagatcaagggctctactgggcacctgtatctggatgtgaagtcctcttctatggacggcaagaagacaaactatgggagagtggattttgccaccttcggagtgagcgtgagcggaaatgctgcaatcaaagggtcccccgtgaaactgaccgctaccggagcaaaagcctttgccggcttttacagagcaggagagcccatgaaccccctgagcaccaatctgacactgagcgctgagaaagtttgtcacaatgtgaccgttgatgcagttaccgggaaagtgatcggcgacgattccggaaagggggcaggaaggggactgcctgtcactagcggaagcattattatccccatcgtggcctctgtgatgatcctgaccgcaatggtcattgtgatcgtgatcggcagcggctga

SEQ ID NO: 310 - RNA, 1377 nt, synthetic construct
  source 1..1377: mol_type = other RNA
atgggagtgaccggaatcctgcagctgcccagagataggttcaagcggaccagcttcttcctgtgggtcattatcctgtttcagagaacattctctgctgggccaacagtgacagtgatccccgtgggacgcgagggcggagacattaccatctccgggaagggctttagcacaacagggttcggtgtgtatgtggctgtggccccagcaagcgtgccagagttctacgggaactccgataaattttatgggtatgaccccagtaaagacacaaccgagagccccagcacaatttgggtttacaccccctctcagaaagctattggctcccggttcgcccagggcagaccaatgaataatgacgggagctttaccatcactatgaaggcccccccattcgagcagggaaaggactttgtggtcctgactaccaaggctcacggagtgggcaaaaccgaccactctgacgacactcggacaccggtgacataccgggaggctactcccgctccaacaggccctaaaactccaatcgcccccagcaagcagccttcaaagcaggctgcccccagcaagcaggtgaagcctagcaagcaggctggccccaacaagcagagcactactcctcagcagaagaccgcagagcacagatctcagaccccagcagctcatcgcactatgactaagcaggtgtgtaccatcggagcctcaaaggtgaccagcggatccctgacatggggaatccggactagctttacttcctacctgcgcgggcccatcgcaaacggcagctggaagctgtctgggggggccaattggaacggcagcgccttcacattcccactgacttctggatccttcgaccccgccaccaaatccggctcactgaagtacagcgggagcgtgcacatgactggacaccatggaattctggacatgacactcgctgaaccctctctgcagatcaagggctctactgggcacctgtatctggatgtgaagtcctcttctatggacggcaagaagacaaactatgggagagtggattttgccaccttcggagtgagcgtgagcggaaatgctgcaatcaaagggtcccccgtgaaactgaccgctaccggagcaaaagcctttgccggcttttacagagcaggagagcccatgaaccccctgagcaccaatctgacactgagcgctgagaaagtttgtcacaatgtgaccgttgatgcagttaccgggaaagtgatcggcgacgattccggaaagggggcaggaaggggactgcctgtcacttctggatcctggatccctgccggaatcggggtgaccggcgtgatcatcgccgtgatcgccctgttctgtattggcagcggatga

SEQ ID NO: 311 - RNA, 51 nt, synthetic construct
  source 1..51: mol_type = other RNA
atgaaagccaagctgctggtgctgctgtgcaccttcaccgccacatacgcc

SEQ ID NO: 312 - RNA, 81 nt, synthetic construct
  source 1..81: mol_type = other RNA
attctggccatctacagcacagtggccagctctctggtgctgctggttagcctgggcgccatcagcttcggcagcggctaa

SEQ ID NO: 313 - protein, 267 aa, Cutibacterium acnes
MKKTHLVAPLLVGAMLVPAALSAPSAHAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVSPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 314 - protein, 267 aa, Cutibacterium acnes
MKKTHLVATLLVGAMLVPAALSAPSAHAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVSPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 315 - protein, 267 aa, Cutibacterium acnes
MKKTHLVTPLLIGAMLVPATLSAPSAHAVEPTTTISATATHELSASDAHNSLQLLNARIATLQSVQKSVPGSDYSDQIRDLLKAAFDLRGLIDTLAHGGIPFYDPSTIMPRIKLAATTIDTIHTATTTLQNKVRPAHVELGLEVTKAVLLTANPASTAKELDAEGAALKARLDKVSQYPDLAPNDVATVYVRTNFSRTIWQVRADRNRYILGHKSAEVYKTLNHAITKAVGVRLNPKTTVGNIQAARTKLLAAYQTAFNSPDVKKAA

SEQ ID NO: 316 - protein, 267 aa, Cutibacterium acnes
MKKTHLVTPLLIGAMLVPATLSAPSAHAVEPTTTISTTATHELSASDAHNSLQLLNARIATLQSVQKSVPGSDYSDQIRDLLKAAFDLRGLIDTLAHGGIPFYDPSTIMPRIKLAATTIDTIHTATTTLQNKVRPAHVELGLEVTKAVLLTANPASTAKELDAEGAALKARLDKVSQYPDLAPNDVATVYVRTNFSRTIWQVRADRNRYILGHKSAEVYKTLNHAITKAVGVRLNPKTTVGNIQAARTKLLAAYQTAFNSPDVKKAA

SEQ ID NO: 317 - protein, 267 aa, Cutibacterium acnes
MKKTHLVAPLLVGAMLVPAALSAPSAHAVEPTTIISATSTHELSASDARNSIQLLNPHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 318 - protein, 267 aa, Cutibacterium acnes
MKKTHLVAPLLVGAMLVPAALSAPSAHAVEPTTTISATSTHELSASYARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPATTAKELDAEGTALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 319 - protein, 267 aa, Cutibacterium acnes
MKKTHLVAPLLVGAMLVPAALSAPSAHAVEPTTTISATSTHELSANEAHNSMQLLNARIATLQSVQKSVPGSDYSDQIRDLLKAAFDLHGLIETLAHGGIPFYDPSTIMPRIKLVATTVDTIHTATTTLQNKVRPAHVELGLEVTKAVLLTANPASTAKELDAEGAALKARLEKVSQYPDLTPNDVATVYVRTNFSKTIWQVRADRDRYILGHKSAAVYKTLNHAITKAVGVRLDPKTTVGNIQAARTELLAAYQTAFNSPDVKKAA

SEQ ID NO: 320 - protein, 267 aa, Cutibacterium acnes
MKKTHLVAPLLVGAMLVPAALSAPSAHAVEPTTTISATSTHELSANEARNSMQLLNARIATLQSVQKSVPGSDYSDQIRDLLKAAFDLRGLIETLTHGGIPFYDPSTIMPRIKLVATTVDTIHTATTTLQNKVRPAHVELGLEVTKAVLLTANPASTAKELDAEGAALKARLEKVSQYPDLTPNDVATVYVRTNFSKTIWQVRADRDRYILGHKSAAVYKTLNHAITKAVGVRLDPKTTVGNIQAARTELLAAYQTAFNSPDVKKAA

SEQ ID NO: 321 - protein, 267 aa, Cutibacterium acnes
MKKTHLVAPLLVGAMLVPAALSAPSAHAVEPTTTISATSTHELSANEARNSMQLLNARIATLQSVQKSVPGSDYSDQIRDLLKAAFDLRGLIETLAHGGIPFYNPSTIMPRIKLVATTVDTIHTATTTLQNKVRPAHVELGLEVTKAVLLTANPASTAKELDAEGAALKARLEKVSQYPDLTPNDVATVYVRTNFSKTIWQVRADRDRYILGHKSAAVYKTLNHAITKAVGVRLDPKTTVGNIQAARTELLAAYQTAFNSPDVKKAA

SEQ ID NO: 322 - protein, 267 aa, Cutibacterium acnes
MKKTHLVAPLLVGAMLVPAALSAPSAHAVEPTTTISATSTHELSANEARNSMQLLNARIATLQSVQKSVPGSDYSDQIRDLLKAAFDLHGLIETLAHGGIPFYDPSTIMPRIKLVATTVDTIHTATTTLQNKVRPAHVELGLEVTKAVLLTANPASTAKELDAEGAALKARLEKVSQYPDLTPNDVATVYVRTNFSKTIWQVRADRDRYILGHKSAAVYKTLNHAITKAVGVRLDPKTTVGNIQAARTELLAAYQTAFNSPDVKKAA

SEQ ID NO: 323 - protein, 267 aa, Cutibacterium acnes
MKKTHLVAPLLVGAMLVPAALSAPSAHAVEPTTTISATSTHELSANEARNSMQLLNARIATLQSVQKSVPGSDYSDQIRDLLKAAFDLRGLIETLAHGGIPFYDPSTIMPRIKLVATTVDTIHTATTTLQNKVRPAHVELGLEVTNAVLLTANPASTAKELDAEGAALKARLEKVSQYPDLTPNDVATVYVRTNFSKTIWQVRADRDRYILGHKSAAVYKTLNHAITKAVGVRLDPKTTVGNIQAARTELLAAYQTAFNSPDVKKAA

SEQ ID NO: 324 - protein, 267 aa, Cutibacterium acnes
MKKTHLVAPLLVGAMLVPAALSAPSAHAVEPTTTISATSTHELSANEARNSMQLLNARIATLQSVQKSVPGSDYSDQIRDLLKAAFDLRGLIETLAHGGIPFYDPSTIMPRIKLVATTVDTIHTATTTLQNKVRPAHVELGLEVTKAVLLTANPASTAKELDAEGAALKAHLEKVSQYPDLTPNDVATVYVRTNFSKTIWQVRADRDRYILGHKSAAVYKTLNHAITKAVGVRLDPKTTVGNIQAARTELLAAYQTAFNSPDVKKAA

SEQ ID NO: 325 - protein, 267 aa, Cutibacterium acnes
MKKTHLVAPLLVGAMLVPAALSAPSAHAVEPTTTISATSTHELSANEARNSMQLLNARIATLQSVQKSVPGSDYSNQIRDLLKAAFDLRGLIETLAHGGIPFYDPSTIMPRIKLVATTVDTIHTAKTILQNKVRPAHVELGLEVTKAVLLTANPASTAKELDAEGAALKARLEKVSQYPDLTPNDVATVYVRTNFSKTIWQVRADRDRYILGHKSAAVYKTLNHAITKAVGVRLDPKTTVGNIQAARTELLAAYQTAFNSPDVKKAA

SEQ ID NO: 326 - protein, 267 aa, Cutibacterium acnes
MKKTHLVAPLLVGAMLVPAALSAPSAHAVEPTTTISATSTHELSASDARNSIQLLNAHIATLQSAQKSVPGSDYSDQIRDLLKAAFDLRGLIETLAHGGIPFYDPSTIMPRIKLVATTIDTIHTATTTLQNKVRPAHVELGLEVTKAVLLTANPATTAKELDAEGTALKARLEKVSQYPDLTPNDVATVYVRTNFSKTIWQVRANRHRYILGHKSAAVYKTLNHAITKAVGVRLNPKTTVGNIQAARTELLAAYQTAFNSPDVKKAA

SEQ ID NO: 327 - protein, 267 aa, Cutibacterium acnes
MKKTHLVAPLLVGAMLVPAALSAPSAHAVEPTTTISATSTHELSASDARNSIQLLNAHIATLQSAQKSVPGSDYSDQIRDLLKAAFDLRGLIETLAHGGIPFYDPSTIMPRIKLVATTIDTIHTATTTLQNKVSPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 328 - protein, 267 aa, Cutibacterium acnes
MKKTHLVAPLLVGAMLVPAALSAPSAHAVEPTTTISATSTHELSASDARNSIQLLNAHIATLQSAQKSVPGSDYSDQIRDLLKAAFDLRGLIETLAHGGIPFYDPSTIMPRIKLVATTIDTIHTATTTLQNKVRPAHVELGLEVTKAVLLTANPATTAKELDAEGTALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 329 - protein, 267 aa, Cutibacterium acnes
MKKTHLVAPLLVGAMLVPAALSAPSAHAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPATTAKELDAEGTALKARLEKVSQYPDLTPNDVATVYVRTNFSKTIWQVRANRDRYILGHKSAAVYKTLNHAITKAVGVRLNPKTTVGNIQAARTELFAAYQTAFNSPDVKKAA

SEQ ID NO: 330 - protein, 267 aa, Cutibacterium acnes
MKKTHLVAPLLVGAMLVPAALSAPSAHAVEPTTTISATSTHELSASDARNSIQLLNAHIATLQSVQKSVPGSDYSDQIRDLLKAAFDLRGLIETLAHGGIPFYDPSTIMPRIKLVATTIGTIHTATTTLQNKVSPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 331 - protein, 267 aa, Cutibacterium acnes
MKKTHLVAPLLVGAMLVPAALSAPSAHAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVSPAHVELGLEVTKAVLLTANPASTAKELDAEGAALKARLKKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 332 - protein, 267 aa, Cutibacterium acnes
MKKTHLVAPLLVGAMLVPAALSAPSAHAVEPTTTISATSTHELSASDARNSIQLLNAHIATLQSVQKSVPGSDYSDQIRDLLKAAFDLRGLIETLAHGGIPFYDPSTIMPRIKLVATTVDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 333 - protein, 267 aa, Cutibacterium acnes
MKKTHLVAPLLVGAMLVPAALSAPSAHAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPATTAKELDAEGTALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 334 - protein, 267 aa, Cutibacterium acnes
MKKTHLVAPLLVGAMLVPAALSAPSAHAVEPTTTISATSTHELSANEARNSMQLLNARIATLQSVQKSVPGSDYSNQIRDLLKAAFDLRGLIETLAHGGIPFYDPSTIMPRIKLVATTVDTIHTATTTLQNKVRPAHVELGLEVTKAVLLTANPASTAKELDAEGAALKARLEKVSQYPDLTPNDVATVYVRTNFSKTIWQVRADRDRYILGHKSAAVYKTLNHAITKAVGVRLDPKTTVGNIQAARTELLAAYQTAFNSPDVKKAA

SEQ ID NO: 335 - protein, 267 aa, Cutibacterium acnes
MKKTHLVAPLLVGAMLVPAALSAPSAHAVEPTTTISATSTHELSASDARNSIQLLNPHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 336 - protein, 267 aa, Cutibacterium acnes
MKKTHLVAPLLVGAMLVPAALSAPSAHAVEPTTTISATSTHELSANEARNSMQLLNARIATLQSVQKSVPGSDYSDQIRDLLKAAFDLRGLIETLAHGGIPFYDPSTIMPRIKLVATTVDTIHTATTTLQNKVRPAHVELGLEVTKAVLLTANPASTAKELDAEGAALKARLEKVSQYPDLTPNDVATVYVRTNFSKTIWQVRADRDRYILGHKSAAVYKTLNHAITKAVGVRLAPKTTVGNIQAARTELLAAYQTAFNSPDVKKAA

SEQ ID NO: 337 - protein, 267 aa, Cutibacterium acnes
MKKTHLVAPLLVGAMLVPAALSAPSAHAVEPTTTISATSTHELSANEARNSMQLLNARIATLQSVQKSVPGSDYSDQIRDLLKAAFDLRGLIETLAHGGIPFYDPSTIMPRIKLVATTVDTIHTATTTLQNKVRPAHVELGLEVTKAVLLTANPASTAKELDAEGAALKARLEKVSQYPDLTPNDVATVYVRTNFSKTIWQVRADRDRYILGHKSAAVYKTLNHAITKAVGVRLDPKTTVGNIQAARTELLAAYQTAFNSPDVKKAA

SEQ ID NO: 338 - protein, 267 aa, Cutibacterium acnes
MKKTHLVAPLLVGAMLVPAALSAPSAHAVEPTTTISATSTHELSASDARNSTQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPATTAKELDAEGTALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 339 - protein, 239 aa, Cutibacterium acnes
VEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVSPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 340 - protein, 239 aa, Cutibacterium acnes
VEPTTTISATATHELSASDAHNSLQLLNARIATLQSVQKSVPGSDYSDQIRDLLKAAFDLRGLIDTLAHGGIPFYDPSTIMPRIKLAATTIDTIHTATTTLQNKVRPAHVELGLEVTKAVLLTANPASTAKELDAEGAALKARLDKVSQYPDLAPNDVATVYVRTNFSRTIWQVRADRNRYILGHKSAEVYKTLNHAITKAVGVRLNPKTTVGNIQAARTKLLAAYQTAFNSPDVKKAA

SEQ ID NO: 341 - protein, 239 aa, Cutibacterium acnes
VEPTTTISTTATHELSASDAHNSLQLLNARIATLQSVQKSVPGSDYSDQIRDLLKAAFDLRGLIDTLAHGGIPFYDPSTIMPRIKLAATTIDTIHTATTTLQNKVRPAHVELGLEVTKAVLLTANPASTAKELDAEGAALKARLDKVSQYPDLAPNDVATVYVRTNFSRTIWQVRADRNRYILGHKSAEVYKTLNHAITKAVGVRLNPKTTVGNIQAARTKLLAAYQTAFNSPDVKKAA

SEQ ID NO: 342 - protein, 239 aa, Cutibacterium acnes
VEPTTIISATSTHELSASDARNSIQLLNPHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 343 - protein, 239 aa, Cutibacterium acnes
VEPTTTISATSTHELSASYARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPATTAKELDAEGTALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 344 - protein, 239 aa, Cutibacterium acnes
VEPTTTISATSTHELSANEAHNSMQLLNARIATLQSVQKSVPGSDYSDQIRDLLKAAFDLHGLIETLAHGGIPFYDPSTIMPRIKLVATTVDTIHTATTTLQNKVRPAHVELGLEVTKAVLLTANPASTAKELDAEGAALKARLEKVSQYPDLTPNDVATVYVRTNFSKTIWQVRADRDRYILGHKSAAVYKTLNHAITKAVGVRLDPKTTVGNIQAARTELLAAYQTAFNSPDVKKAA

SEQ ID NO: 345 - protein, 239 aa, Cutibacterium acnes
VEPTTTISATSTHELSANEARNSMQLLNARIATLQSVQKSVPGSDYSDQIRDLLKAAFDLRGLIETLTHGGIPFYDPSTIMPRIKLVATTVDTIHTATTTLQNKVRPAHVELGLEVTKAVLLTANPASTAKELDAEGAALKARLEKVSQYPDLTPNDVATVYVRTNFSKTIWQVRADRDRYILGHKSAAVYKTLNHAITKAVGVRLDPKTTVGNIQAARTELLAAYQTAFNSPDVKKAA

SEQ ID NO: 346 - protein, 239 aa, Cutibacterium acnes
VEPTTTISATSTHELSANEARNSMQLLNARIATLQSVQKSVPGSDYSDQIRDLLKAAFDLRGLIETLAHGGIPFYNPSTIMPRIKLVATTVDTIHTATTTLQNKVRPAHVELGLEVTKAVLLTANPASTAKELDAEGAALKARLEKVSQYPDLTPNDVATVYVRTNFSKTIWQVRADRDRYILGHKSAAVYKTLNHAITKAVGVRLDPKTTVGNIQAARTELLAAYQTAFNSPDVKKAA

SEQ ID NO: 347 - protein, 239 aa, Cutibacterium acnes
VEPTTTISATSTHELSANEARNSMQLLNARIATLQSVQKSVPGSDYSDQIRDLLKAAFDLHGLIETLAHGGIPFYDPSTIMPRIKLVATTVDTIHTATTTLQNKVRPAHVELGLEVTKAVLLTANPASTAKELDAEGAALKARLEKVSQYPDLTPNDVATVYVRTNFSKTIWQVRADRDRYILGHKSAAVYKTLNHAITKAVGVRLDPKTTVGNIQAARTELLAAYQTAFNSPDVKKAA

SEQ ID NO: 348 - protein, 239 aa, Cutibacterium acnes
VEPTTTISATSTHELSANEARNSMQLLNARIATLQSVQKSVPGSDYSDQIRDLLKAAFDLRGLIETLAHGGIPFYDPSTIMPRIKLVATTVDTIHTATTTLQNKVRPAHVELGLEVTNAVLLTANPASTAKELDAEGAALKARLEKVSQYPDLTPNDVATVYVRTNFSKTIWQVRADRDRYILGHKSAAVYKTLNHAITKAVGVRLDPKTTVGNIQAARTELLAAYQTAFNSPDVKKAA

SEQ ID NO: 349 - protein, 239 aa, Cutibacterium acnes
VEPTTTISATSTHELSANEARNSMQLLNARIATLQSVQKSVPGSDYSDQIRDLLKAAFDLRGLIETLAHGGIPFYDPSTIMPRIKLVATTVDTIHTATTTLQNKVRPAHVELGLEVTKAVLLTANPASTAKELDAEGAALKAHLEKVSQYPDLTPNDVATVYVRTNFSKTIWQVRADRDRYILGHKSAAVYKTLNHAITKAVGVRLDPKTTVGNIQAARTELLAAYQTAFNSPDVKKAA

SEQ ID NO: 350 - protein, 239 aa, Cutibacterium acnes
VEPTTTISATSTHELSANEARNSMQLLNARIATLQSVQKSVPGSDYSNQIRDLLKAAFDLRGLIETLAHGGIPFYDPSTIMPRIKLVATTVDTIHTAKTILQNKVRPAHVELGLEVTKAVLLTANPASTAKELDAEGAALKARLEKVSQYPDLTPNDVATVYVRTNFSKTIWQVRADRDRYILGHKSAAVYKTLNHAITKAVGVRLDPKTTVGNIQAARTELLAAYQTAFNSPDVKKAA

SEQ ID NO: 351 - protein, 239 aa, Cutibacterium acnes
VEPTTTISATSTHELSASDARNSIQLLNAHIATLQSAQKSVPGSDYSDQIRDLLKAAFDLRGLIETLAHGGIPFYDPSTIMPRIKLVATTIDTIHTATTTLQNKVRPAHVELGLEVTKAVLLTANPATTAKELDAEGTALKARLEKVSQYPDLTPNDVATVYVRTNFSKTIWQVRANRHRYILGHKSAAVYKTLNHAITKAVGVRLNPKTTVGNIQAARTELLAAYQTAFNSPDVKKAA

SEQ ID NO: 352 - protein, 239 aa, Cutibacterium acnes
VEPTTTISATSTHELSASDARNSIQLLNAHIATLQSAQKSVPGSDYSDQIRDLLKAAFDLRGLIETLAHGGIPFYDPSTIMPRIKLVATTIDTIHTATTTLQNKVSPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 353 - protein, 239 aa, Cutibacterium acnes
VEPTTTISATSTHELSASDARNSIQLLNAHIATLQSAQKSVPGSDYSDQIRDLLKAAFDLRGLIETLAHGGIPFYDPSTIMPRIKLVATTIDTIHTATTTLQNKVRPAHVELGLEVTKAVLLTANPATTAKELDAEGTALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 354 - protein, 239 aa, Cutibacterium acnes
VEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPATTAKELDAEGTALKARLEKVSQYPDLTPNDVATVYVRTNFSKTIWQVRANRDRYILGHKSAAVYKTLNHAITKAVGVRLNPKTTVGNIQAARTELFAAYQTAFNSPDVKKAA

SEQ ID NO: 355 - protein, 239 aa, Cutibacterium acnes
VEPTTTISATSTHELSASDARNSIQLLNAHIATLQSVQKSVPGSDYSDQIRDLLKAAFDLRGLIETLAHGGIPFYDPSTIMPRIKLVATTIGTIHTATTTLQNKVSPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 356 - protein, 239 aa, Cutibacterium acnes
VEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVSPAHVELGLEVTKAVLLTANPASTAKELDAEGAALKARLKKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 357 - protein, 239 aa, Cutibacterium acnes
VEPTTTISATSTHELSASDARNSIQLLNAHIATLQSVQKSVPGSDYSDQIRDLLKAAFDLRGLIETLAHGGIPFYDPSTIMPRIKLVATTVDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 358 - protein, 239 aa, Cutibacterium acnes
VEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPATTAKELDAEGTALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 359 - protein, 239 aa, Cutibacterium acnes
VEPTTTISATSTHELSANEARNSMQLLNARIATLQSVQKSVPGSDYSNQIRDLLKAAFDLRGLIETLAHGGIPFYDPSTIMPRIKLVATTVDTIHTATTTLQNKVRPAHVELGLEVTKAVLLTANPASTAKELDAEGAALKARLEKVSQYPDLTPNDVATVYVRTNFSKTIWQVRADRDRYILGHKSAAVYKTLNHAITKAVGVRLDPKTTVGNIQAARTELLAAYQTAFNSPDVKKAA

SEQ ID NO: 360 - protein, 239 aa, Cutibacterium acnes
VEPTTTISATSTHELSASDARNSIQLLNPHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 361 - protein, 239 aa, Cutibacterium acnes
VEPTTTISATSTHELSANEARNSMQLLNARIATLQSVQKSVPGSDYSDQIRDLLKAAFDLRGLIETLAHGGIPFYDPSTIMPRIKLVATTVDTIHTATTTLQNKVRPAHVELGLEVTKAVLLTANPASTAKELDAEGAALKARLEKVSQYPDLTPNDVATVYVRTNFSKTIWQVRADRDRYILGHKSAAVYKTLNHAITKAVGVRLAPKTTVGNIQAARTELLAAYQTAFNSPDVKKAA

SEQ ID NO: 362 - protein, 239 aa, Cutibacterium acnes
VEPTTTISATSTHELSANEARNSMQLLNARIATLQSVQKSVPGSDYSDQIRDLLKAAFDLRGLIETLAHGGIPFYDPSTIMPRIKLVATTVDTIHTATTTLQNKVRPAHVELGLEVTKAVLLTANPASTAKELDAEGAALKARLEKVSQYPDLTPNDVATVYVRTNFSKTIWQVRADRDRYILGHKSAAVYKTLNHAITKAVGVRLDPKTTVGNIQAARTELLAAYQTAFNSPDVKKAA

SEQ ID NO: 363 - protein, 239 aa, Cutibacterium acnes
VEPTTTISATSTHELSASDARNSTQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPATTAKELDAEGTALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 364-366 - skipped (3 entries)

SEQ ID NO: 367 - protein, 445 aa, synthetic construct
MKAKLLVLLCTFTATYAVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKAITRQIVDVITADKAIKTAEQLEKELPALDDLVKKAMGPPKPTGGGGGAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKGDHGDDTRTPVGYR

SEQ ID NO: 368 - protein, 428 aa, synthetic construct
VAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKAITRQIVDVITADKAIKTAEQLEKELPALDDLVKKAMGPPKPTGGGGGAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKGDHGDDTRTPVGYR

SEQ ID NO: 369 - protein, 736 aa, synthetic construct
MKAKLLVLLCTFTATYAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAASSNRPRSVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKASSPPKPTPTPTPTPTAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPISGSILAIYSTVASSLVLLVSLGAISFGSG

SEQ ID NO: 370 - protein, 707 aa, synthetic construct
MKAKLLVLLCTFTATYAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAASSNRPRSVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKASSPPKPTPTPTPTPTAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPI

SEQ ID NO: 371 - protein, 628 aa, synthetic construct
MKAKLLVLLCTFTATYAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATSSNRPRSVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKASSPPKPTPTPTPTPTAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPI

SEQ ID NO: 372 - protein, 547 aa, synthetic construct
MKAKLLVLLCTFTATYASSNRPRSVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKASSPPKPTPTPTPTPTAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIVYVRTNFSKTIWQVRANRDRYILGHKSAAVYKTLNHAITKAVGVRLNPKTTVGNIQAARTELLAAYQTAFNSPDVKKAA

SEQ ID NO: 373 - protein, 690 aa, synthetic construct
VEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAASSNRPRSVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKASSPPKPTPTPTPTPTAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPI

SEQ ID NO: 374 - protein, 690 aa, synthetic construct; identical to SEQ ID NO: 373

SEQ ID NO: 375 - protein, 611 aa, synthetic construct
VEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATSSNRPRSVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKASSPPKPTPTPTPTPTAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPI

SEQ ID NO: 376 - protein, 530 aa, synthetic construct
SSNRPRSVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKASSPPKPTPTPTPTPTAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIVYVRTNFSKTIWQVRANRDRYILGHKSAAVYKTLNHAITKAVGVRLNPKTTVGNIQAARTELLAAYQTAFNSPDVKKAA

SEQ ID NO: 377 - RNA, 2214 nt, synthetic construct
  source 1..2214: mol_type = other RNA
atgaaggccaagctgctggtgctgctgtgtaccttcactgcaacatatgctgtggagcccacaaccacaatctctgccacaagcacccacgagctgtctgccagcgacgccagaaactccatccagctgctgaatgcccacatcgccacactgcagagcgtgcagaaatccgtgcctggcagcgattacagcgaccagatccgggacctgctgaaggccgcctttgatctgagaggcctgatcgaaaccctggcccacggcggcattcccttctatgacccctccaccatcatgcccagaatcaagctggtggccaccaccatcgacaccatccacacagctacaaccaccctgcagaacaaggtgcggccagcccatgtggaactgggcctggaggtgacaaaggccgtgctgctgaccgccaatcctgccagcacagcaaaagaactggacgccgagggagctgctctgaaggccagactggaaaaggtgagccagtaccccgacctgacacctaatgatgtggccacagtgtacgtgagaaccaatttcagcaagaccatctggcaggtgagagccaacagagatcggtacatcctgggccacaagtccgccgccgtgtacaagaccctgaatcacgccatcaccaaggccgtgggcgtgcggctgaatcctaagaccaccgtgggcaacatccaggccgccagaaccgaactgctggccgcctaccagaccgccttcaacagccccgatgtgaagaaggccgctagctccaatagacccagatccgtggcccaggccgccatcgccacagatggcaaggggatcatcgacaaggattccagagacgctgtgatcaacgacgcaaagctgagagccgccatcgccggagctctggtgaaggccggctttagcagcgccgacgccgtggcactggcccccagaattgcaaaggagatggcaaaggagggcgtgctgctgatcaatcaccacaagctgaaggccctgatcggcgcacagctgggcctgctgaccgacgcaaagattcagagagccgccgcagccgtggacctgggcatcaaggccaccctggctgccaccatcatccctaacgccctgggcagcgccgccttcaagaatgcagtgatcgccaacctggtggccgccggaatcgacaagcacctggccagagccacagccgtcgctatcgtggccacagccctgaatcccgccctgggccccatcgccaaatttgaactgatcaaggcagaaatcgctgcccaggccgccctgctcatcagaagaggcgtccatctgcagaaggccgccatcgagcacgtgattggcagagccttcgacgccgccgtggccacagctatcatttcttcccctatcctgtctgccagaatcgtgacacacctggtgagagccggaatcgacaagtccatcgccatctccctggccccccacatcgtgaagagactggctaaggagcctctgctggccttcaatacagccaagctgatgaagaacatcacaagacagatcgtggacgtgatcaccgccgacaaggccatcaagacagccgagcagctggagaaggaactgccagccctggacgacctggtgaagaaggccagcagccctcccaagcctacccctaccccaacccctacacctacagccggacctaccgtgaccgtgatccctgtgggcagagagggcggcgacatcaccatcagcggcaagggcttcagcacaacaggctttggcgtgtacgtggccgtggccccagccagcgtgcctgagttttacggcaatagcgacaagttctacggctacgacccctccaaggacaccaccgagagcccaagcaccatctgggtgtacacacctagccagaaggccatcggcagcagatttgcccagggcaggcctatgaataatgacggcagcttcacaatcacaatgaaggcccccccctttgagcagggcaaggacttcgtggtgctgaccaccaaggcccacggcgtgggcaagacagatcacagcgacgacacccggacccccgtgacctacagagaagcaacacctgcccctaccgggcctaagacacccatcagcggcagcatcctggctatctacagcacagtggcttctagcctggtgctgctggtgtctctgggcgccatcagctttggcagcggctgataa

SEQ ID NO: 378 - RNA, 2214 nt, synthetic construct
  source 1..2214: mol_type = other RNA
atgaaggctaagctgctggtgctgctgtgcacattcacagccacctacgccgtggagcctaccaccacaatcagcgccaccagcacccacgagctgagcgccagcgatgccagaaatagcatccagctgctgaatgcccacatcgccaccctgcagtccgtgcagaaaagcgtgcctggaagcgattacagcgatcagatcagggacctgctgaaggccgccttcgatctgagaggcctgatcgagacactggcccacggcggcatccctttctacgaccccagcaccatcatgcctagaatcaaactggtggccaccaccatcgacaccatccacaccgcaacaacaaccctgcagaataaggtgcggcctgcccacgtggaactgggactggaagtgacaaaggccgtgctgctgaccgctaatcccgcctccaccgccaaggagctggatgccgaaggagctgccctgaaagccagactggagaaggtgagccagtaccctgatctgacccctaacgacgtggccaccgtgtacgtgagaaccaatttcagcaagaccatctggcaggtgagagccaatcgggacagatacatcctgggccacaagtctgccgccgtgtataaaaccctgaatcacgccatcaccaaggccgtgggcgtgagactgaaccctaagacaaccgtgggcaacatccaggccgccagaacagagctgctggccgcctaccagacagccttcaatagccccgacgtgaagaaggccgcttccagcaatagaccccggagcgtggcacaggctgctattgccaccgacggaaagggcatcatcgataaagacagccgggatgccgtgatcaatgacgccaagctgagagcagcaatcgccggcgctctggtgaaggcaggcttcagcagcgccgacgccgtggctctggcccctagaatcgctaaggagatggccaaggagggcgtgctgctgatcaatcaccacaagctgaaggccctgatcggcgcccagctgggactgctgacagatgccaagatccagagagccgctgcagccgtggatctgggcatcaaggccacactggccgccaccatcatcccaaacgccctgggcagcgccgccttcaaaaatgccgtgattgccaacctggtggccgccggcatcgataagcacctggccagagccacagccgtggccatcgtggccacagccctgaatcccgcactgggacctattgccaagttcgaactcattaaggccgaaatcgcagcccaagccgccctgctgattcggcggggcgtgcacctgcagaaagccgccattgagcacgtgatcggcagagccttcgatgccgctgtggccaccgccatcatctcctctcctatcctgtctgctagaatcgtgacccacctggtcagagccggaatcgacaagtctatcgctatcagcctggccccccacatcgtgaagagactggccaaagagcccctgctggccttcaacacagccaagctcatgaagaatatcaccagacagatcgtggacgtgatcaccgccgacaaggccatcaaaaccgctgagcagctggagaaggagctgcccgccctggatgacctggtgaagaaggccagcagcccccccaagcctacacctacacctaccccaacccctacagccggccctacagtgaccgtgattcctgtgggcagagaaggaggcgacatcaccatcagcggaaagggcttctctaccacaggcttcggcgtgtacgtggccgtggcccctgcctccgtgcccgaattctacggcaacagcgacaagttctacggctacgaccccagcaaagacaccacagagagcccttccaccatctgggtgtacaccccttcccagaaggccatcggcagcagattcgctcagggcagacctatgaataacgacggctctttcaccatcacaatgaaggccccccccttcgagcagggcaaagacttcgtggtgctgacaaccaaggcccacggcgtgggcaagacagatcactccgacgacacccggacccctgtgacctacagagaggcaacacctgctcccaccgggccaaagacccctatcagcggaagcatcctggccatctatagcaccgtggctagctctctggtgctgctggtgagcctgggagctatcagctttgggagcggctgataa

SEQ ID NO: 379 - RNA, 2130 nt, synthetic construct
  source 1..2130: mol_type = other RNA
atgaaggccaagctgctggtgctgctgtgtaccttcactgcaacatatgctatggtggagcccacaaccacaatctctgccacaagcacccacgagctgtctgccagcgacgccagaaactccatccagctgctgaatgcccacatcgccacactgcagagcgtgcagaaatccgtgcctggcagcgattacagcgaccagatccgggacctgctgaaggccgcctttgatctgagaggcctgatcgaaaccctggcccacggcggcattcccttctatgacccctccaccatcatgcccagaatcaagctggtggccaccaccatcgacaccatccacacagctacaaccaccctgcagaacaaggtgcggccagcccatgtggaactgggcctggaggtgacaaaggccgtgctgctgaccgccaatcctgccagcacagcaaaagaactggacgccgagggagctgctctgaaggccagactggaaaaggtgagccagtaccccgacctgacacctaatgatgtggccacagtgtacgtgagaaccaatttcagcaagaccatctggcaggtgagagccaacagagatcggtacatcctgggccacaagtccgccgccgtgtacaagaccctgaatcacgccatcaccaaggccgtgggcgtgcggctgaatcctaagaccaccgtgggcaacatccaggccgccagaaccgaactgctggccgcctaccagaccgccttcaacagccccgatgtgaagaaggccgctagctccaatagacccagatccgtggcccaggccgccatcgccacagatggcaaggggatcatcgacaaggattccagagacgctgtgatcaacgacgcaaagctgagagccgccatcgccggagctctggtgaaggccggctttagcagcgccgacgccgtggcactggcccccagaattgcaaaggagatggcaaaggagggcgtgctgctgatcaatcaccacaagctgaaggccctgatcggcgcacagctgggcctgctgaccgacgcaaagattcagagagccgccgcagccgtggacctgggcatcaaggccaccctggctgccaccatcatccctaacgccctgggcagcgccgccttcaagaatgcagtgatcgccaacctggtggccgccggaatcgacaagcacctggccagagccacagccgtcgctatcgtggccacagccctgaatcccgccctgggccccatcgccaaatttgaactgatcaaggcagaaatcgctgcccaggccgccctgctcatcagaagaggcgtccatctgcagaaggccgccatcgagcacgtgattggcagagccttcgacgccgccgtggccacagctatcatttcttcccctatcctgtctgccagaatcgtgacacacctggtgagagccggaatcgacaagtccatcgccatctccctggccccccacatcgtgaagagactggctaaggagcctctgctggccttcaatacagccaagctgatgaagaacatcacaagacagatcgtggacgtgatcaccgccgacaaggccatcaagacagccgagcagctggagaaggaactgccagccctggacgacctggtgaagaaggccagcagccctcccaagcctacccctaccccaacccctacacctacagccggacctaccgtgaccgtgatccctgtgggcagagagggcggcgacatcaccatcagcggcaagggcttcagcacaacaggctttggcgtgtacgtggccgtggccccagccagcgtgcctgagttttacggcaatagcgacaagttctacggctacgacccctccaaggacaccaccgagagcccaagcaccatctgggtgtacacacctagccagaaggccatcggcagcagatttgcccagggcaggcctatgaataatgacggcagcttcacaatcacaatgaaggcccccccctttgagcagggcaaggacttcgtggtgctgaccaccaaggcccacggcgtgggcaagacagatcacagcgacgacacccggacccccgtgacctacagagaagcaacacctgcccctaccgggcctaagacacccatctgataa

SEQ ID NO: 380 - RNA, 2127 nt, synthetic construct
  source 1..2127: mol_type = other RNA
atgaaggctaagctgctggtgctgctgtgcacattcacagccacctacgccgtggagcctaccaccacaatcagcgccaccagcacccacgagctgagcgccagcgatgccagaaatagcatccagctgctgaatgcccacatcgccaccctgcagtccgtgcagaaaagcgtgcctggaagcgattacagcgatcagatcagggacctgctgaaggccgccttcgatctgagaggcctgatcgagacactggcccacggcggcatccctttctacgaccccagcaccatcatgcctagaatcaaactggtggccaccaccatcgacaccatccacaccgcaacaacaaccctgcagaataaggtgcggcctgcccacgtggaactgggactggaagtgacaaaggccgtgctgctgaccgctaatcccgcctccaccgccaaggagctggatgccgaaggagctgccctgaaagccagactggagaaggtgagccagtaccctgatctgacccctaacgacgtggccaccgtgtacgtgagaaccaatttcagcaagaccatctggcaggtgagagccaatcgggacagatacatcctgggccacaagtctgccgccgtgtataaaaccctgaatcacgccatcaccaaggccgtgggcgtgagactgaaccctaagacaaccgtgggcaacatccaggccgccagaacagagctgctggccgcctaccagacagccttcaatagccccgacgtgaagaaggccgcttccagcaatagaccccggagcgtggcacaggctgctattgccaccgacggaaagggcatcatcgataaagacagccgggatgccgtgatcaatgacgccaagctgagagcagcaatcgccggcgctctggtgaaggcaggcttcagcagcgccgacgccgtggctctggcccctagaatcgctaaggagatggccaaggagggcgtgctgctgatcaatcaccacaagctgaaggccctgatcggcgcccagctgggactgctgacagatgccaagatccagagagccgctgcagccgtggatctgggcatcaaggccacactggccgccaccatcatcccaaacgccctgggcagcgccgccttcaaaaatgccgtgattgccaacctggtggccgccggcatcgataagcacctggccagagccacagccgtggccatcgtggccacagccctgaatcccgcactgggacctattgccaagttcgaactcattaaggccgaaatcgcagcccaagccgccctgctgattcggcggggcgtgcacctgcagaaagccgccattgagcacgtgatcggcagagccttcgatgccgctgtggccaccgccatcatctcctctcctatcctgtctgctagaatcgtgacccacctggtcagagccggaatcgacaagtctatcgctatcagcctggccccccacatcgtgaagagactggccaaagagcccctgctggccttcaacacagccaagctcatgaagaatatcaccagacagatcgtggacgtgatcaccgccgacaaggccatcaaaaccgctgagcagctggagaaggagctgcccgccctggatgacctggtgaagaaggccagcagcccccccaagcctacacctacacctaccccaacccctacagccggccctacagtgaccgtgattcctgtgggcagagaaggaggcgacatcaccatcagcggaaagggcttctctaccacaggcttcggcgtgtacgtggccgtggcccctgcctccgtgcccgaattctacggcaacagcgacaagttctacggctacgaccccagcaaagacaccacagagagcccttccaccatctgggtgtacaccccttcccagaaggccatcggcagcagattcgctcagggcagacctatgaataacgacggctctttcaccatcacaatgaaggccccccccttcgagcagggcaaagacttcgtggtgctgacaaccaaggcccacggcgtgggcaagacagatcactccgacgacacccggacccctgtgacctacagagaggcaacacctgctcccaccgggccaaagacccctatctgataa

SEQ ID NO: 381 - RNA, 1890 nt, synthetic construct
  source 1..1890: mol_type = other RNA
atgaaggccaagctgctggtgctgctgtgtaccttcactgcaacatatgctgtggagcccacaaccacaatctctgccacaagcacccacgagctgtctgccagcgacgccagaaactccatccagctgctgaatgcccacatcgccacactgcagagcgtgcagaaatccgtgcctggcagcgattacagcgaccagatccgggacctgctgaaggccgcctttgatctgagaggcctgatcgaaaccctggcccacggcggcattcccttctatgacccctccaccatcatgcccagaatcaagctggtggccaccaccatcgacaccatccacacagctacaaccaccctgcagaacaaggtgcggccagcccatgtggaactgggcctggaggtgacaaaggccgtgctgctgaccgccaatcctgccagcacagcaaaagaactggacgccgagggagctgctctgaaggccagactggaaaaggtgagccagtaccccgacctgacacctaatgatgtggccacaagctccaatagacccagatccgtggcccaggccgccatcgccacagatggcaaggggatcatcgacaaggattccagagacgctgtgatcaacgacgcaaagctgagagccgccatcgccggagctctggtgaaggccggctttagcagcgccgacgccgtggcactggcccccagaattgcaaaggagatggcaaaggagggcgtgctgctgatcaatcaccacaagctgaaggccctgatcggcgcacagctgggcctgctgaccgacgcaaagattcagagagccgccgcagccgtggacctgggcatcaaggccaccctggctgccaccatcatccctaacgccctgggcagcgccgccttcaagaatgcagtgatcgccaacctggtggccgccggaatcgacaagcacctggccagagccacagccgtcgctatcgtggccacagccctgaatcccgccctgggccccatcgccaaatttgaactgatcaaggcagaaatcgctgcccaggccgccctgctcatcagaagaggcgtccatctgcagaaggccgccatcgagcacgtgattggcagagccttcgacgccgccgtggccacagctatcatttcttcccctatcctgtctgccagaatcgtgacacacctggtgagagccggaatcgacaagtccatcgccatctccctggccccccacatcgtgaagagactggctaaggagcctctgctggccttcaatacagccaagctgatgaagaacatcacaagacagatcgtggacgtgatcaccgccgacaaggccatcaagacagccgagcagctggagaaggaactgccagccctggacgacctggtgaagaaggccagcagccctcccaagcctacccctaccccaacccctacacctacagccggacctaccgtgaccgtgatccctgtgggcagagagggcggcgacatcaccatcagcggcaagggcttcagcacaacaggctttggcgtgtacgtggccgtggccccagccagcgtgcctgagttttacggcaatagcgacaagttctacggctacgacccctccaaggacaccaccgagagcccaagcaccatctgggtgtacacacctagccagaaggccatcggcagcagatttgcccagggcaggcctatgaataatgacggcagcttcacaatcacaatgaaggcccccccctttgagcagggcaaggacttcgtggtgctgaccaccaaggcccacggcgtgggcaagacagatcacagcgacgacacccggacccccgtgacctacagagaagcaacacctgcccctaccgggcctaagacacccatctgataa

SEQ ID NO: 382 - RNA, 1890 nt, synthetic construct
  source 1..1890: mol_type = other RNA
atgaaggctaagctgctggtgctgctgtgcacattcacagccacctacgccgtggagcctaccaccacaatcagcgccaccagcacccacgagctgagcgccagcgatgccagaaatagcatccagctgctgaatgcccacatcgccaccctgcagtccgtgcagaaaagcgtgcctggaagcgattacagcgatcagatcagggacctgctgaaggccgccttcgatctgagaggcctgatcgagacactggcccacggcggcatccctttctacgaccccagcaccatcatgcctagaatcaaactggtggccaccaccatcgacaccatccacaccgcaacaacaaccctgcagaataaggtgcggcctgcccacgtggaactgggactggaagtgacaaaggccgtgctgctgaccgctaatcccgcctccaccgccaaggagctggatgccgaaggagctgccctgaaagccagactggagaaggtgagccagtaccctgatctgacccctaacgacgtggccacctccagcaatagaccccggagcgtggcacaggctgctattgccaccgacggaaagggcatcatcgataaagacagccgggatgccgtgatcaatgacgccaagctgagagcagcaatcgccggcgctctggtgaaggcaggcttcagcagcgccgacgccgtggctctggcccctagaatcgctaaggagatggccaaggagggcgtgctgctgatcaatcaccacaagctgaaggccctgatcggcgcccagctgggactgctgacagatgccaagatccagagagccgctgcagccgtggatctgggcatcaaggccacactggccgccaccatcatcccaaacgccctgggcagcgccgccttcaaaaatgccgtgattgccaacctggtggccgccggcatcgataagcacctggccagagccacagccgtggccatcgtggccacagccctgaatcccgcactgggacctattgccaagttcgaactcattaaggccgaaatcgcagcccaagccgccctgctgattcggcggggcgtgcacctgcagaaagccgccattgagcacgtgatcggcagagccttcgatgccgctgtggccaccgccatcatctcctctcctatcctgtctgctagaatcgtgacccacctggtcagagccggaatcgacaagtctatcgctatcagcctggccccccacatcgtgaagagactggccaaagagcccctgctggccttcaacacagccaagctcatgaagaatatcaccagacagatcgtggacgtgatcaccgccgacaaggccatcaaaaccgctgagcagctggagaaggagctgcccgccctggatgacctggtgaagaaggccagcagcccccccaagcctacacctacacctaccccaacccctacagccggccctacagtgaccgtgattcctgtgggcagagaaggaggcgacatcaccatcagcggaaagggcttctctaccacaggcttcggcgtgtacgtggccgtggcccctgcctccgtgcccgaattctacggcaacagcgacaagttctacggctacgaccccagcaaagacaccacagagagcccttccaccatctgggtgtacaccccttcccagaaggccatcggcagcagattcgctcagggcagacctatgaataacgacggctctttcaccatcacaatgaaggccccccccttcgagcagggcaaagacttcgtggtgctgacaaccaaggcccacggcgtgggcaagacagatcactccgacgacacccggacccctgtgacctacagagaggcaacacctgctcccaccgggccaaagacccctatctgataa

SEQ ID NO: 383 - RNA, 1647 nt, synthetic construct
  source 1..1647: mol_type = other RNA
atgaaggctaagctgctggtgctgctgtgcacattcacagccacctacgcctccagcaatagaccccggagcgtggcacaggctgctattgccaccgacggaaagggcatcatcgataaagacagccgggatgccgtgatcaatgacgccaagctgagagcagcaatcgccggcgctctggtgaaggcaggcttcagcagcgccgacgccgtggctctggcccctagaatcgctaaggagatggccaaggagggcgtgctgctgatcaatcaccacaagctgaaggccctgatcggcgcccagctgggactgctgacagatgccaagatccagagagccgctgcagccgtggatctgggcatcaaggccacactggccgccaccatcatcccaaacgccctgggcagcgccgccttcaaaaatgccgtgattgccaacctggtggccgccggcatcgataagcacctggccagagccacagccgtggccatcgtggccacagccctgaatcccgcactgggacctattgccaagttcgaactcattaaggccgaaatcgcagcccaagccgccctgctgattcggcggggcgtgcacctgcagaaagccgccattgagcacgtgatcggcagagccttcgatgccgctgtggccaccgccatcatctcctctcctatcctgtctgctagaatcgtgacccacctggtcagagccggaatcgacaagtctatcgctatcagcctggccccccacatcgtgaagagactggccaaagagcccctgctggccttcaacacagccaagctcatgaagaatatcaccagacagatcgtggacgtgatcaccgccgacaaggccatcaaaaccgctgagcagctggagaaggagctgcccgccctggatgacctggtgaagaaggccagcagcccccccaagcctacacctacacctaccccaacccctacagccggccctacagtgaccgtgattcctgtgggcagagaaggaggcgacatcaccatcagcggaaagggcttctctaccacaggcttcggcgtgtacgtggccgtggcccctgcctccgtgcccgaattctacggcaacagcgacaagttctacggctacgaccccagcaaagacaccacagagagcccttccaccatctgggtgtacaccccttcccagaaggccatcggcagcagattcgctcagggcagacctatgaataacgacggctctttcaccatcacaatgaaggccccccccttcgagcagggcaaagacttcgtggtgctgacaaccaaggcccacggcgtgggcaagacagatcactccgacgacacccggacccctgtgacctacagagaggcaacacctgctcccaccgggccaaagacccctatcgtgtacgtgagaaccaatttcagcaagaccatctggcaggtgagagccaatcgggacagatacatcctgggccacaagtctgccgccgtgtataaaaccctgaatcacgccatcaccaaggccgtgggcgtgagactgaaccctaagacaaccgtgggcaacatccaggccgccagaacagagctgctggccgcctaccagacagccttcaatagccccgacgtgaagaaggccgcttgataa

SEQ ID NO: 384 - RNA, 2070 nt, synthetic construct
  source 1..2070: mol_type = other RNA
gtggagcccacaaccacaatctctgccacaagcacccacgagctgtctgccagcgacgccagaaactccatccagctgctgaatgcccacatcgccacactgcagagcgtgcagaaatccgtgcctggcagcgattacagcgaccagatccgggacctgctgaaggccgcctttgatctgagaggcctgatcgaaaccctggcccacggcggcattcccttctatgacccctccaccatcatgcccagaatcaagctggtggccaccaccatcgacaccatccacacagctacaaccaccctgcagaacaaggtgcggccagcccatgtggaactgggcctggaggtgacaaaggccgtgctgctgaccgccaatcctgccagcacagcaaaagaactggacgccgagggagctgctctgaaggccagactggaaaaggtgagccagtaccccgacctgacacctaatgatgtggccacagtgtacgtgagaaccaatttcagcaagaccatctggcaggtgagagccaacagagatcggtacatcctgggccacaagtccgccgccgtgtacaagaccctgaatcacgccatcaccaaggccgtgggcgtgcggctgaatcctaagaccaccgtgggcaacatccaggccgccagaaccgaactgctggccgcctaccagaccgccttcaacagccccgatgtgaagaaggccgctagctccaatagacccagatccgtggcccaggccgccatcgccacagatggcaaggggatcatcgacaaggattccagagacgctgtgatcaacgacgcaaagctgagagccgccatcgccggagctctggtgaaggccggctttagcagcgccgacgccgtggcactggcccccagaattgcaaaggagatggcaaaggagggcgtgctgctgatcaatcaccacaagctgaaggccctgatcggcgcacagctgggcctgctgaccgacgcaaagattcagagagccgccgcagccgtggacctgggcatcaaggccaccctggctgccaccatcatccctaacgccctgggcagcgccgccttcaagaatgcagtgatcgccaacctggtggccgccggaatcgacaagcacctggccagagccacagccgtcgctatcgtggccacagccctgaatcccgccctgggccccatcgccaaatttgaactgatcaaggcagaaatcgctgcccaggccgccctgctcatcagaagaggcgtccatctgcagaaggccgccatcgagcacgtgattggcagagccttcgacgccgccgtggccacagctatcatttcttcccctatcctgtctgccagaatcgtgacacacctggtgagagccggaatcgacaagtccatcgccatctccctggccccccacatcgtgaagagactggctaaggagcctctgctggccttcaatacagccaagctgatgaagaacatcacaagacagatcgtggacgtgatcaccgccgacaaggccatcaagacagccgagcagctggagaaggaactgccagccctggacgacctggtgaagaaggccagcagccctcccaagcctacccctaccccaacccctacacctacagccggacctaccgtgaccgtgatccctgtgggcagagagggcggcgacatcaccatcagcggcaagggcttcagcacaacaggctttggcgtgtacgtggccgtggccccagccagcgtgcctgagttttacggcaatagcgacaagttctacggctacgacccctccaaggacaccaccgagagcccaagcaccatctgggtgtacacacctagccagaaggccatcggcagcagatttgcccagggcaggcctatgaataatgacggcagcttcacaatcacaatgaaggcccccccctttgagcagggcaaggacttcgtggtgctgaccaccaaggcccacggcgtgggcaagacagatcacagcgacgacacccggacccccgtgacctacagagaagcaacacctgcccctaccgggcctaagacacccatc

SEQ ID NO: 385 - RNA, 2070 nt, synthetic construct
  source 1..2070: mol_type = other RNA
gtggagcctaccaccacaatcagcgccaccagcacccacgagctgagcgccagcgatgccagaaatagcatccagctgctgaatgcccacatcgccaccctgcagtccgtgcagaaaagcgtgcctggaagcgattacagcgatcagatcagggacctgctgaaggccgccttcgatctgagaggcctgatcgagacactggcccacggcggcatccctttctacgaccccagcaccatcatgcctagaatcaaactggtggccaccaccatcgacaccatccacaccgcaacaacaaccctgcagaataaggtgcggcctgcccacgtggaactgggactggaagtgacaaaggccgtgctgctgaccgctaatcccgcctccaccgccaaggagctggatgccgaaggagctgccctgaaagccagactggagaaggtgagccagtaccctgatctgacccctaacgacgtggccaccgtgtacgtgagaaccaatttcagcaagaccatctggcaggtgagagccaatcgggacagatacatcctgggccacaagtctgccgccgtgtataaaaccctgaatcacgccatcaccaaggccgtgggcgtgagactgaaccctaagacaaccgtgggcaacatccaggccgccagaacagagctgctggccgcctaccagacagccttcaatagccccgacgtgaagaaggccgcttccagcaatagaccccggagcgtggcacaggctgctattgccaccgacggaaagggcatcatcgataaagacagccgggatgccgtgatcaatgacgccaagctgagagcagcaatcgccggcgctctggtgaaggcaggcttcagcagcgccgacgccgtggctctggcccctagaatcgctaaggagatggccaaggagggcgtgctgctgatcaatcaccacaagctgaaggccctgatcggcgcccagctgggactgctgacagatgccaagatccagagagccgctgcagccgtggatctgggcatcaaggccacactggccgccaccatcatcccaaacgccctgggcagcgccgccttcaaaaatgccgtgattgccaacctggtggccgccggcatcgataagcacctggccagagccacagccgtggccatcgtggccacagccctgaatcccgcactgggacctattgccaagttcgaactcattaaggccgaaatcgcagcccaagccgccctgctgattcggcggggcgtgcacctgcagaaagccgccattgagcacgtgatcggcagagccttcgatgccgctgtggccaccgccatcatctcctctcctatcctgtctgctagaatcgtgacccacctggtcagagccggaatcgacaagtctatcgctatcagcctggccccccacatcgtgaagagactggccaaagagcccctgctggccttcaacacagccaagctcatgaagaatatcaccagacagatcgtggacgtgatcaccgccgacaaggccatcaaaaccgctgagcagctggagaaggagctgcccgccctggatgacctggtgaagaaggccagcagcccccccaagcctacacctacacctaccccaacccctacagccggccctacagtgaccgtgattcctgtgggcagagaaggaggcgacatcaccatcagcggaaagggcttctctaccacaggcttcggcgtgtacgtggccgtggcccctgcctccgtgcccgaattctacggcaacagcgacaagttctacggctacgaccccagcaaagacaccacagagagcccttccaccatctgggtgtacaccccttcccagaaggccatcggcagcagattcgctcagggcagacctatgaataacgacggctctttcaccatcacaatgaaggccccccccttcgagcagggcaaagacttcgtggtgctgacaaccaaggcccacggcgtgggcaagacagatcactccgacgacacccggacccctgtgacctacagagaggcaacacctgctcccaccgggccaaagacccctatc

SEQ ID NO: 386 - RNA, 2073 nt, synthetic construct
  source 1..2073: mol_type = other RNA
atggtggagcccacaaccacaatctctgccacaagcacccacgagctgtctgccagcgacgccagaaactccatccagctgctgaatgcccacatcgccacactgcagagcgtgcagaaatccgtgcctggcagcgattacagcgaccagatccgggacctgctgaaggccgcctttgatctgagaggcctgatcgaaaccctggcccacggcggcattcccttctatgacccctccaccatcatgcccagaatcaagctggtggccaccaccatcgacaccatccacacagctacaaccaccctgcagaacaaggtgcggccagcccatgtggaactgggcctggaggtgacaaaggccgtgctgctgaccgccaatcctgccagcacagcaaaagaactggacgccgagggagctgctctgaaggccagactggaaaaggtgagccagtaccccgacctgacacctaatgatgtggccacagtgtacgtgagaaccaatttcagcaagaccatctggcaggtgagagccaacagagatcggtacatcctgggccacaagtccgccgccgtgtacaagaccctgaatcacgccatcaccaaggccgtgggcgtgcggctgaatcctaagaccaccgtgggcaacatccaggccgccagaaccgaactgctggccgcctaccagaccgccttcaacagccccgatgtgaagaaggccgctagctccaatagacccagatccgtggcccaggccgccatcgccacagatggcaaggggatcatcgacaaggattccagagacgctgtgatcaacgacgcaaagctgagagccgccatcgccggagctctggtgaaggccggctttagcagcgccgacgccgtggcactggcccccagaattgcaaaggagatggcaaaggagggcgtgctgctgatcaatcaccacaagctgaaggccctgatcggcgcacagctgggcctgctgaccgacgcaaagattcagagagccgccgcagccgtggacctgggcatcaaggccaccctggctgccaccatcatccctaacgccctgggcagcgccgccttcaagaatgcagtgatcgccaacctggtggccgccggaatcgacaagcacctggccagagccacagccgtcgctatcgtggccacagccctgaatcccgccctgggccccatcgccaaatttgaactgatcaaggcagaaatcgctgcccaggccgccctgctcatcagaagaggcgtccatctgcagaaggccgccatcgagcacgtgattggcagagccttcgacgccgccgtggccacagctatcatttcttcccctatcctgtctgccagaatcgtgacacacctggtgagagccggaatcgacaagtccatcgccatctccctggccccccacatcgtgaagagactggctaaggagcctctgctggccttcaatacagccaagctgatgaagaacatcacaagacagatcgtggacgtgatcaccgccgacaaggccatcaagacagccgagcagctggagaaggaactgccagccctggacgacctggtgaagaaggccagcagccctcccaagcctacccctaccccaacccctacacctacagccggacctaccgtgaccgtgatccctgtgggcagagagggcggcgacatcaccatcagcggcaagggcttcagcacaacaggctttggcgtgtacgtggccgtggccccagccagcgtgcctgagttttacggcaatagcgacaagttctacggctacgacccctccaaggacaccaccgagagcccaagcaccatctgggtgtacacacctagccagaaggccatcggcagcagatttgcccagggcaggcctatgaataatgacggcagcttcacaatcacaatgaaggcccccccctttgagcagggcaaggacttcgtggtgctgaccaccaaggcccacggcgtgggcaagacagatcacagcgacgacacccggacccccgtgacctacagagaagcaacacctgcccctaccgggcctaagacacccatc

SEQ ID NO: 387 - RNA, 2070 nt, synthetic construct; identical to SEQ ID NO: 385
  source 1..2070: mol_type = other RNA

SEQ ID NO: 388 - RNA, 1833 nt, synthetic construct
  source 1..1833: mol_type = other RNA
gtggagcccacaaccacaatctctgccacaagcacccacgagctgtctgccagcgacgccagaaactccatccagctgctgaatgcccacatcgccacactgcagagcgtgcagaaatccgtgcctggcagcgattacagcgaccagatccgggacctgctgaaggccgcctttgatctgagaggcctgatcgaaaccctggcccacggcggcattcccttctatgacccctccaccatcatgcccagaatcaagctggtggccaccaccatcgacaccatccacacagctacaaccaccctgcagaacaaggtgcggccagcccatgtggaactgggcctggaggtgacaaaggccgtgctgctgaccgccaatcctgccagcacagcaaaagaactggacgccgagggagctgctctgaaggccagactggaaaaggtgagccagtaccccgacctgacacctaatgatgtggccacaagctccaatagacccagatccgtggcccaggccgccatcgccacagatggcaaggggatcatcgacaaggattccagagacgctgtgatcaacgacgcaaagctgagagccgccatcgccggagctctggtgaaggccggctttagcagcgccgacgccgtggcactggcccccagaattgcaaaggagatggcaaaggagggcgtgctgctgatcaatcaccacaagctgaaggccctgatcggcgcacagctgggcctgctgaccgacgcaaagattcagagagccgccgcagccgtggacctgggcatcaaggccaccctggctgccaccatcatccctaacgccctgggcagcgccgccttcaagaatgcagtgatcgccaacctggtggccgccggaatcgacaagcacctggccagagccacagccgtcgctatcgtggccacagccctgaatcccgccctgggccccatcgccaaatttgaactgatcaaggcagaaatcgctgcccaggccgccctgctcatcagaagaggcgtccatctgcagaaggccgccatcgagcacgtgattggcagagccttcgacgccgccgtggccacagctatcatttcttcccctatcctgtctgccagaatcgtgacacacctggtgagagccggaatcgacaagtccatcgccatctccctggccccccacatcgtgaagagactggctaaggagcctctgctggccttcaatacagccaagctgatgaagaacatcacaagacagatcgtggacgtgatcaccgccgacaaggccatcaagacagccgagcagctggagaaggaactgccagccctggacgacctggtgaagaaggccagcagccctcccaagcctacccctaccccaacccctacacctacagccggacctaccgtgaccgtgatccctgtgggcagagagggcggcgacatcaccatcagcggcaagggcttcagcacaacaggctttggcgtgtacgtggccgtggccccagccagcgtgcctgagttttacggcaatagcgacaagttctacggctacgacccctccaaggacaccaccgagagcccaagcaccatctgggtgtacacacctagccagaaggccatcggcagcagatttgcccagggcaggcctatgaataatgacggcagcttcacaatcacaatgaaggcccccccctttgagcagggcaaggacttcgtggtgctgaccaccaaggcccacggcgtgggcaagacagatcacagcgacgacacccggacccccgtgacctacagagaagcaacacctgcccctaccgggcctaagacacccatc

SEQ ID NO: 389 - RNA, 1833 nt, synthetic construct
  source 1..1833: mol_type = other RNA
gtggagcctaccaccacaatcagcgccaccagcacccacgagctgagcgccagcgatgccagaaatagcatccagctgctgaatgcccacatcgccaccctgcagtccgtgcagaaaagcgtgcctggaagcgattacagcgatcagatcagggacctgctgaaggccgccttcgatctgagaggcctgatcgagacactggcccacggcggcatccctttctacgaccccagcaccatcatgcctagaatcaaactggtggccaccaccatcgacaccatccacaccgcaacaacaaccctgcagaataaggtgcggcctgcccacgtggaactgggactggaagtgacaaaggccgtgctgctgaccgctaatcccgcctccaccgccaaggagctggatgccgaaggagctgccctgaaagccagactggagaaggtgagccagtaccctgatctgacccctaacgacgtggccacctccagcaatagaccccggagcgtggcacaggctgctattgccaccgacggaaagggcatcatcgataaagacagccgggatgccgtgatcaatgacgccaagctgagagcagcaatcgccggcgctctggtgaaggcaggcttcagcagcgccgacgccgtggctctggcccctagaatcgctaaggagatggccaaggagggcgtgctgctgatcaatcaccacaagctgaaggccctgatcggcgcccagctgggactgctgacagatgccaagatccagagagccgctgcagccgtggatctgggcatcaaggccacactggccgccaccatcatcccaaacgccctgggcagcgccgccttcaaaaatgccgtgattgccaacctggtggccgccggcatcgataagcacctggccagagccacagccgtggccatcgtggccacagccctgaatcccgcactgggacctattgccaagttcgaactcattaaggccgaaatcgcagcccaagccgccctgctgattcggcggggcgtgcacctgcagaaagccgccattgagcacgtgatcggcagagccttcgatgccgctgtggccaccgccatcatctcctctcctatcctgtctgctagaatcgtgacccacctggtcagagccggaatcgacaagtctatcgctatcagcctggccccccacatcgtgaagagactggccaaagagcccctgctggccttcaacacagccaagctcatgaagaatatcaccagacagatcgtggacgtgatcaccgccgacaaggccatcaaaaccgctgagcagctggagaaggagctgcccgccctggatgacctggtgaagaaggccagcagcccccccaagcctacacctacacctaccccaacccctacagccggccctacagtgaccgtgattcctgtgggcagagaaggaggcgacatcaccatcagcggaaagggcttctctaccacaggcttcggcgtgtacgtggccgtggcccctgcctccgtgcccgaattctacggcaacagcgacaagttctacggctacgaccccagcaaagacaccacagagagcccttccaccatctgggtgtacaccccttcccagaaggccatcggcagcagattcgctcagggcagacctatgaataacgacggctctttcaccatcacaatgaaggccccccccttcgagcagggcaaagacttcgtggtgctgacaaccaaggcccacggcgtgggcaagacagatcactccgacgacacccggacccctgtgacctacagagaggcaacacctgctcccaccgggccaaagacccctatc

SEQ ID NO: 390 - RNA, 1590 nt, synthetic construct
  source 1..1590: mol_type = other RNA
tccagcaatagaccccggagcgtggcacaggctgctattgccaccgacggaaagggcatcatcgataaagacagccgggatgccgtgatcaatgacgccaagctgagagcagcaatcgccggcgctctggtgaaggcaggcttcagcagcgccgacgccgtggctctggcccctagaatcgctaaggagatggccaaggagggcgtgctgctgatcaatcaccacaagctgaaggccctgatcggcgcccagctgggactgctgacagatgccaagatccagagagccgctgcagccgtggatctgggcatcaaggccacactggccgccaccatcatcccaaacgccctgggcagcgccgccttcaaaaatgccgtgattgccaacctggtggccgccggcatcgataagcacctggccagagccacagccgtggccatcgtggccacagccctgaatcccgcactgggacctattgccaagttcgaactcattaaggccgaaatcgcagcccaagccgccctgctgattcggcggggcgtgcacctgcagaaagccgccattgagcacgtgatcggcagagccttcgatgccgctgtggccaccgccatcatctcctctcctatcctgtctgctagaatcgtgacccacctggtcagagccggaatcgacaagtctatcgctatcagcctggccccccacatcgtgaagagactggccaaagagcccctgctggccttcaacacagccaagctcatgaagaatatcaccagacagatcgtggacgtgatcaccgccgacaaggccatcaaaaccgctgagcagctggagaaggagctgcccgccctggatgacctggtgaagaaggccagcagcccccccaagcctacacctacacctaccccaacccctacagccggccctacagtgaccgtgattcctgtgggcagagaaggaggcgacatcaccatcagcggaaagggcttctctaccacaggcttcggcgtgtacgtggccgtggcccctgcctccgtgcccgaattctacggcaacagcgacaagttctacggctacgaccccagcaaagacaccacagagagcccttccaccatctgggtgtacaccccttcccagaaggccatcggcagcagattcgctcagggcagacctatgaataacgacggctctttcaccatcacaatgaaggccccccccttcgagcagggcaaagacttcgtggtgctgacaaccaaggcccacggcgtgggcaagacagatcactccgacgacacccggacccctgtgacctacagagaggcaacacctgctcccaccgggccaaagacccctatcgtgtacgtgagaaccaatttcagcaagaccatctggcaggtgagagccaatcgggacagatacatcctgggccacaagtctgccgccgtgtataaaaccctgaatcacgccatcaccaaggccgtgggcgtgagactgaaccctaagacaaccgtgggcaacatccaggccgccagaacagagctgctggccgcctaccagacagccttcaatagccccgacgtgaagaaggccgct

SEQ ID NO: 391 - RNA, 858 nt, synthetic construct
  source 1..858: mol_type = other RNA
atgaaggccaagctgctggtgctgctgtgcaccttcaccgccacctacgctgtggagcccaccaccaccatcagcgcaacctccacccacgaactgtccgcctccgatgctaggaactcaatccagctgctgaacgcccacatcgccacactgcagagcgtccagaaatcagtgcccggcagcgactactccgaccagatccgggatctcctgaaggctgcattcgacctgcgcggcctcatcgaaacactggcccacggaggcatccccttctatgacccatccaccatcatgccccggatcaagctggtggccaccacaatcgacaccatccacaccgccaccaccaccctgcagaacaaggtgaggcccgcacacgtggagctggggctggaggtgactaaggccgtgctgctgacagcaaaccccgcctcaaccgcgaaggagttggatgccgagggcgccgcactgaaggcccgcctggagaaggtgagccagtatcccgatctgacccccaacgacgtggccaccgtgtatgtgaggaccaatttctccaagaccatctggcaggtcagagccaacagagacaggtatatcctgggccacaagagcgctgccgtgtacaagaccctgaaccacgccatcaccaaggccgtgggggtgcggctgaaccctaagaccaccgtggggaacatccaggctgccagaaccgagctgctggccgcctatcagaccgcattcaattcccccgatgtgaagaaggccgccagcggctccatcctggccatctactccaccgtggctagtagcctggtgctgctggtgtccctgggagccatcagcttcgggagcggctaa

SEQ ID NO: 392 - RNA, 858 nt, synthetic construct
  source 1..858: mol_type = other RNA
atgaaagccaagctgctcgtgctgctgtgtaccttcaccgccacctacgccgtggaacccaccaccacaatctccgccacctccacccacgagctgagcgccagcgacgccagaaacagcattcagctgctgaatgcccacatcgccaccctgcagagcgtgcagaagtccgtgcctggcagcgactacagcgaccagatccgggacctgctgaaggccgccttcgacctgagaggcctgattgagacactggcccacggcggcattcctttctacgatccttctaccatcatgcctagaatcaagctggtggccaccaccatcgataccatccacacagccaccaccacactgcagaacaaggtgcggcctgctcacgtggaactgggcctggaagtgaccaaggccgtgctgctgaccgccaatcctgcctccaccgccaaggagctggacgcagaaggcgctgccctgaaggccagactggagaaggtgagccagtatcccgacctgaccccaaacgatgtggccaccgtgtacgtgcggacaaacttcagcaagaccatctggcaggtgagagccaaccgggacagatacatcctgggccacaagagcgccgccgtgtataagaccctgaaccacgccatcacaaaggccgtgggcgtgagactgaaccccaagaccacagtgggcaacatccaggccgccaggaccgaactgctggccgcctaccagaccgccttcaattcccctgacgtgaagaaggccgcctctggcagcatcctggccatctacagcaccgtggccagctctctggtgctgctggtgtctctgggcgccatcagcttcggcagcggctga

SEQ ID NO: 393 - RNA, 1590 nt, synthetic construct
  source 1..1590: mol_type = other RNA
agctccaatagacccagatccgtggcccaggccgccatcgccacagatggcaaggggatcatcgacaaggattccagagacgctgtgatcaacgacgcaaagctgagagccgccatcgccggagctctggtgaaggccggctttagcagcgccgacgccgtggcactggcccccagaattgcaaaggagatggcaaaggagggcgtgctgctgatcaatcaccacaagctgaaggccctgatcggcgcacagctgggcctgctgaccgacgcaaagattcagagagccgccgcagccgtggacctgggcatcaaggccaccctggctgccaccatcatccctaacgccctgggcagcgccgccttcaagaatgcagtgatcgccaacctggtggccgccggaatcgacaagcacctggccagagccacagccgtcgctatcgtggccacagccctgaatcccgccctgggccccatcgccaaatttgaactgatcaaggcagaaatcgctgcccaggccgccctgctcatcagaagaggcgtccatctgcagaaggccgccatcgagcacgtgattggcagagccttcgacgccgccgtggccacagctatcatttcttcccctatcctgtctgccagaatcgtgacacacctggtgagagccggaatcgacaagtccatcgccatctccctggccccccacatcgtgaagagactggctaaggagcctctgctggccttcaatacagccaagctgatgaagaacatcacaagacagatcgtggacgtgatcaccgccgacaaggccatcaagacagccgagcagctggagaaggaactgccagccctggacgacctggtgaagaaggccagcagccctcccaagcctacccctaccccaacccctacacctacagccggacctaccgtgaccgtgatccctgtgggcagagagggcggcgacatcaccatcagcggcaagggcttcagcacaacaggctttggcgtgtacgtggccgtggccccagccagcgtgcctgagttttacggcaatagcgacaagttctacggctacgacccctccaaggacaccaccgagagcccaagcaccatctgggtgtacacacctagccagaaggccatcggcagcagatttgcccagggcaggcctatgaataatgacggcagcttcacaatcacaatgaaggcccccccctttgagcagggcaaggacttcgtggtgctgaccaccaaggcccacggcgtgggcaagacagatcacagcgacgacacccggacccccgtgacctacagagaagcaacacctgcccctaccgggcctaagacacccatcgtgtacgtgagaaccaatttcagcaagaccatctggcaggtgagagccaacagagatcggtacatcctgggccacaagtccgccgccgtgtacaagaccctgaatcacgccatcaccaaggccgtgggcgtgcggctgaatcctaagaccaccgtgggcaacatccaggccgccagaaccgaactgctggccgcctaccagaccgccttcaacagccccgatgtgaagaaggccgct

SEQ ID NO: 394 - RNA, 1647 nt, synthetic construct
  source 1..1647: mol_type = other RNA
atgaaggccaagctgctggtgctgctgtgtaccttcactgcaacatatgctagctccaatagacccagatccgtggcccaggccgccatcgccacagatggcaaggggatcatcgacaaggattccagagacgctgtgatcaacgacgcaaagctgagagccgccatcgccggagctctggtgaaggccggctttagcagcgccgacgccgtggcactggcccccagaattgcaaaggagatggcaaaggagggcgtgctgctgatcaatcaccacaagctgaaggccctgatcggcgcacagctgggcctgctgaccgacgcaaagattcagagagccgccgcagccgtggacctgggcatcaaggccaccctggctgccaccatcatccctaacgccctgggcagcgccgccttcaagaatgcagtgatcgccaacctggtggccgccggaatcgacaagcacctggccagagccacagccgtcgctatcgtggccacagccctgaatcccgccctgggccccatcgccaaatttgaactgatcaaggcagaaatcgctgcccaggccgccctgctcatcagaagaggcgtccatctgcagaaggccgccatcgagcacgtgattggcagagccttcgacgccgccgtggccacagctatcatttcttcccctatcctgtctgccagaatcgtgacacacctggtgagagccggaatcgacaagtccatcgccatctccctggccccccacatcgtgaagagactggctaaggagcctctgctggccttcaatacagccaagctgatgaagaacatcacaagacagatcgtggacgtgatcaccgccgacaaggccatcaagacagccgagcagctggagaaggaactgccagccctggacgacctggtgaagaaggccagcagccctcccaagcctacccctaccccaacccctacacctacagccggacctaccgtgaccgtgatccctgtgggcagagagggcggcgacatcaccatcagcggcaagggcttcagcacaacaggctttggcgtgtacgtggccgtggccccagccagcgtgcctgagttttacggcaatagcgacaagttctacggctacgacccctccaaggacaccaccgagagcccaagcaccatctgggtgtacacacctagccagaaggccatcggcagcagatttgcccagggcaggcctatgaataatgacggcagcttcacaatcacaatgaaggcccccccctttgagcagggcaaggacttcgtggtgctgaccaccaaggcccacggcgtgggcaagacagatcacagcgacgacacccggacccccgtgacctacagagaagcaacacctgcccctaccgggcctaagacacccatcgtgtacgtgagaaccaatttcagcaagaccatctggcaggtgagagccaacagagatcggtacatcctgggccacaagtccgccgccgtgtacaagaccctgaatcacgccatcaccaaggccgtgggcgtgcggctgaatcctaagaccaccgtgggcaacatccaggccgccagaaccgaactgctggccgcctaccagaccgccttcaacagccccgatgtgaagaaggccgcttgataa

SEQ ID NO: 395 - RNA, 87 nt, synthetic construct
  source 1..87: mol_type = other RNA
agcggcagcatcctggctatctacagcacagtggcttctagcctggtgctgctggtgtctctgggcgccatcagctttggcagcggc

SEQ ID NO: 396 - RNA, 87 nt, synthetic construct
  source 1..87: mol_type = other RNA
agcggaagcatcctggccatctatagcaccgtggctagctctctggtgctgctggtgagcctgggagctatcagctttgggagcggc

SEQ ID NO: 397 - skipped

SEQ ID NO: 398 - RNA, 858 nt, synthetic construct
  source 1..858: mol_type = other RNA
atgaargccaagctgctsgtgctgctstgyacyttyaccgccacctaygcygtsgarcccaccacmacaatcwgcgcmacmtccacmcacgarctgwscgcmwccgaygcyagraaywsmatycagctgctgaaygcccacatcgccacmctscagagcgtscagaarwshgtgccyggcwsygaytaywscgaccagatccgggayctsctsaaggcygcmttygacctgmgmggcctsatygaracactggcycayggmggsatyccyttctaygaycchwsyaccatcatgccymgratcaagctggtsgccachacmatcgayaccatccacacmgccaccaccacmctgcagaacaargtgmgsccygchcaygtggarctgggsctggargtgacyaargcsgtgctgctgacmgcmaayccygchwsmacmgcsaaggarytggaygcmgarggmgcygcmctgaaggcymgvctsgagaaggtgwsccagtaycccgayctgaccccmaaygaygtggcmacygtgtaygtgmgracmaayttcwscaagaccatytggcaggtsmgggccaaymgrgacagrtayatcctgggscacaaragcgcygccgtgtayaagaccctgaaycacgccatcacmaaggccgtgggvgtgmgrctgaaycchaagaccacmgtgggsaayatycaggcygccmgracmgarctgctggccgcctaycagacmgchttyaatwscccygaygtgaagaaggccgccwsyggswscatyctggccatctacwsyaccgtggcywsywsyctggtgctgctggtgwsyctgggmgcyatywscttcggvwsyggstra

SEQ ID NO: 399 - RNA, 858 nt, synthetic construct
  source 1..858: mol_type = other RNA
atgaargccaagctgctsgtgctgctstgyacyttyaccgccacctaygccgtsgarcccaccacmacaatcwgcgccacmtccacmcacgarctgwscgcmwccgacgccagaaaywscatycagctgctgaaygcccacatcgccaccctscagagcgtgcagaarwsygtgccyggcwsygaytayagcgaccagatccgggacctgctsaaggccgcmttygacctgmgmggcctgatygagacactggcycayggmggsatyccyttctaygatccywsyaccatcatgccymgratcaagctggtsgccachacmatcgayaccatccacacmgccaccaccacmctgcagaacaargtgcgsccygcycaygtggarctgggsctggargtgaccaargcsgtgctgctgacmgccaaycctgchwscacmgccaaggarctggaygcmgarggmgcygcmctgaaggcymgrctsgagaaggtgwsccagtaycccgacctgaccccmaaygaygtggcmacygtgtacgtgmgracmaayttcagcaagaccatytggcaggtsmgggccaaymgrgacagatacatcctgggscacaaragcgccgccgtgtayaagaccctgaaycacgccatcacmaaggccgtgggmgtgmgrctgaaycchaagaccacmgtgggcaayatycaggccgccmgracmgarctgctggccgcctaccagacmgcyttyaatwscccygaygtgaagaaggccgccwsyggsagcatyctggccatctacwsyaccgtggccwsywsyctggtgctgctggtgwsyctgggmgcyatywscttcggvwsyggstra